(19) United States
(12) Patent Application Publication
(10) Pub. No.: US 2019/0103560 A1
(43) Pub. Date: Apr. 4, 2019

(54) COMPOUND AND ORGANIC LIGHT EMITTING DEVICE COMPRISING THE SAME

(71) Applicant: SOULBRAIN CO., LTD., Seongnam-si (KR)

(72) Inventors: Kwang Ju JUNG, Seongnam-si (KR); Seok Jong LEE, Seongnam-si (KR); Eun Chul SHIN, Seongnam-si (KR); Jin Hee KIM, Seongnam-si (KR)

(21) Appl. No.: 16/205,231

(22) Filed: Nov. 30, 2018

Related U.S. Application Data
(63) Continuation of application No. PCT/KR2017/015592, filed on Dec. 27, 2017.

(30) Foreign Application Priority Data
Dec. 27, 2016 (KR) 10-2016-0179927

(51) Int. Cl. H01L 51/00; C07D 491/048; C09K 11/06; C07D 519/00; C07F 9/6561; C07D 495/04

(52) U.S. Cl. CPC H01L 51/0071; C07D 491/048; H01L 51/0067; C09K 11/06; H01L 51/5016; C07F 9/6561; H01L 51/0072; C07D 495/04; C07D 519/00

(57) ABSTRACT

The present invention provides a compound represented by Chemical Formula 1, and a light emitting device comprising the same.

### CROSS-REFERENCE TO RELATED APPLICATIONS

This application claims priority under 35 U.S.C. § 119 to Korean Patent Application No. 10-2016-0179927, filed on Dec. 27, 2016, in the Korean Intellectual Property Office, the disclosure of which is incorporated herein by reference in its entirety.

### TECHNICAL FIELD

The following disclosure relates to a compound and an organic light emitting device including the same.

### BACKGROUND

In general, the organic light emitting phenomenon refers to a phenomenon in which electric energy is converted into light energy through use of an organic material. In the manufacture of organic electroluminescent devices using organic materials, the devices manufactured have gradually expanded their areas of application to the fields of display and illumination for various electronic products; however, efficiency and lifetime are limiting factors in expanding the fields of use, and thus a number of studies are underway in view of both the material used as well as the device in order to improve the efficiency and lifetime. The host material which is being used simultaneously with a dopant material is also important to obtain high light emitting efficiency and lifetime characteristics. As a light emitting host material, in view of the light emitting mechanism, phosphorescent materials, rather than fluorescent materials, have been actively studied as a method for improving the efficiency. For example, carbazole derivatives including 4,4′-bis(9-carbazolyl)biphenyl (CBP) material, which is a representative material, are being used. When a device is manufactured using a carbazole derivative material such as CBP as the phosphorescent light emitting host material, the electron or hole transporting ability is biased toward one side, and thus the efficiency of light emitting is poor, driving voltage increases, whereby there is no great benefit even in view of power efficiency, while the lifetime is also unsatisfactory. Therefore, in order for organic electroluminescent devices to sufficiently exhibit excellent characteristics thereof, the hole injecting material, hole transport material, light emitting material, electron transport material, electron injecting material, and the like, included in the device are required to be supported by stable and efficient materials.

Patent Literature: JP 2008-214244

Patent Literature: JP 2003-133075

### SUMMARY

An embodiment of the present invention is directed to providing a compound capable of being used as a material of an organic material layer of an organic light emitting device, and an organic light emitting device comprising the same.

An embodiment of the present invention provides a compound represented by Chemical Formula 1 below:

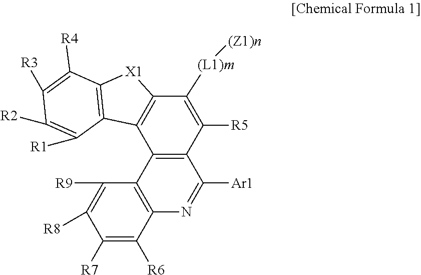

In Chemical Formula 1,

X1 is S or O,

L1 is a direct bond; a substituted or unsubstituted arylene group; or a substituted or unsubstituted heteroarylene group,

Z1 is hydrogen; a substituted or unsubstituted N-containing heterocyclic group; a substituted or unsubstituted amine group; or —P(═O)RaRb,

m is an integer of 0 to 4, and each L1 may be the same as or different from each other when m is 2 or higher,

n is an integer of 1 to 4, and each Z1 may be the same as or different from each other when n is 2 or higher,

Ar1 is a substituted or unsubstituted aryl group; or a substituted or unsubstituted heteroaryl group,

R1 to R9, Ra, and Rb are the same as or different from each other, and each independently selected from the group consisting of: hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group substituted or unsubstituted with an alkyl group, an aryl group, or a heteroaryl group, where R, R′, and R″ may be the same as or different from each other, and each, independently: hydrogen; deuterium; —CN; a substituted or unsubstituted alkyl group; a substituted or unsubstituted cycloalkyl group; a substituted or unsubstituted aryl group; or a substituted or unsubstituted heteroaryl group.

In addition, another embodiment of the present invention provides an organic light emitting diode comprising an anode, a cathode, and one or more layered organic material layers provided between the anode and the cathode, wherein one or more of the organic material layers include the compound represented by Chemical Formula 1.

### BRIEF DESCRIPTION OF THE DRAWINGS

FIG. 1 is a schematic view showing the stacked structure of an organic light emitting device according to an embodiment of the present invention.

### DETAILED DESCRIPTION OF EMBODIMENTS

Hereinafter, the present invention will be described in detail.

In the present specification, the term “substitution” means that a hydrogen atom bonded to a carbon atom of the compound is substituted with another substituent, and the position to be substituted is not limited if it is a position where the hydrogen atom is substituted, i.e., a position where substitution with the substituent is possible. When substitution is carried out with two or more substituents, the two or more substituents may be the same as or different from each other.

In the present specification, the halogen may be fluorine, chlorine, bromine or iodine.

In the present specification, the term alkyl group includes linear or branched chains having 1 to 60 carbon atoms, which may be further substituted with other substituents. The number of carbon atoms of the alkyl group may be 1 to 60, preferably 1 to 40, and more preferably 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but the alkyl group is not limited thereto.

In the present specification, the alkenyl group includes linear or branched chains having 2 to 60 carbon atoms, which may be additionally substituted with other substituents. The number of carbon atoms of the alkenyl group may be 2 to 60, preferably 2 to 40, and more preferably 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but the alkenyl group is not limited thereto.

In the present specification, the alkynyl group includes straight chain or branched chains having 2 to 60 carbon atoms, which may be additionally substituted with other substituents. The number of carbon atoms of the alkynyl group may be 2 to 60, preferably 2 to 40, and more preferably 2 to 20.

In the present specification, the cycloalkyl group includes monocyclic or polycyclic groups having 3 to 60 carbon atoms, which may be further substituted with other substituents. Here, the term “polycyclic” means a group in which a cycloalkyl group is directly linked to another ring group or condensed therewith. Here, the other ring group may be a cycloalkyl group, but may also be another kind of ring group such as a heterocycloalkyl group, an aryl group, a heteroaryl group, and the like. The number of carbon atoms of the cycloalkyl group may be 3 to 60, preferably 3 to 40, and more preferably 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but the cycloalkyl group is not limited thereto.

In the present specification, the heterocycloalkyl group includes O, S, Se, N or Si as a hetero atom and includes a monocyclic or polycyclic group having 2 to 60 carbon atoms, which may be further substituted with other substituents. Here, the term “polycyclic” means a group in which a heterocycloalkyl group is directly linked to another ring group or condensed therewith. Here, the other cyclic group may also be a heterocycloalkyl group, but it may also be another kind of cyclic group, for example, a cycloalkyl group, an aryl group, a heteroaryl group, and the like. The number of carbon atoms of the heterocycloalkyl group may be 2 to 60, preferably 2 to 40, and more preferably 3 to 20.

In the present specification, the aryl group includes monocyclic or polycyclic groups having 6 to 60 carbon atoms, which may be additionally substituted with other substituents. Here, a polycyclic group means a group in which an aryl group is directly linked to or fused with another cyclic group. Here, the other cyclic group may also be an aryl group, but it may also be another kind of cyclic group, for example, a cycloalkyl group, a heterocycloalkyl group, a heteroaryl group, and the like. The term aryl group also includes spiro groups. The number of carbon atoms of the aryl group may be 6 to 60, preferably 6 to 40, and more preferably 6 to 25. Specific examples of the aryl group include a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but the aryl group is not limited thereto.

In the present specification, the spiro group is a group including a spiro structure, and it may have 15 to 60 carbon atoms. For example, the spiro group may include structures in which a 2,3-dihydro-1H-indene group or a cyclohexane group is spiro-bonded to a fluorenyl group.

In the present specification, the heteroaryl group includes O, S, Se, N, or Si as a heteroatom, includes monocycles or polycycles having 2 to 60 carbon atoms, and may be additionally substituted with other substituents. Here, polycycle means a group in which a heteroaryl group is directly linked to or fused with another cyclic group. Here, the other cyclic group may also be a heteroaryl group, but it may also be another kind of cyclic group, for example, a cycloalkyl group, a heterocycloalkyl group, an aryl group, and the like. The number of carbon atoms of the heteroaryl group may be 2 to 60, preferably 2 to 40, and more preferably 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a triazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a triazinyl group, a dioxinyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but the heteroaryl group is not limited thereto.

In the present specification, the amine group may be selected from the group consisting of a monoalkylamine group, a monoarylamine group, a monoheteroarylamine group, —NH2, a dialkylamine group, a diarylamine group, a diheteroarylamine group, an alkylarylamine group, an alkylheteroarylamine group, and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but the amine group is not limited thereto.

As used herein, the term “arylene group” refers to a group having two bonding positions in an aryl group, i.e., a bivalent group. The description of the aryl group described above may be applied to these groups, except that each of these groups are bivalent. Further, the term heteroarylene group refers to a group having two bonding positions in a heteroaryl group, i.e., a bivalent group. The description of the heteroaryl group described above may be applied to these groups, except that each of these groups are bivalent.

In the present specification, examples of structures exemplified by the above-described aryl group and heteroaryl group may be applied, except that the hydrocarbon ring and the hetero ring formed by adjacent groups are not monovalent.

The term “substituted or unsubstituted” as used herein means substitution or unsubstitution with one or more substituents selected from the group consisting of: deuterium; a halogen group; —CN; a C₁-C₆₀ alkyl group; a C₂-C₆₀ alkenyl group; a C₂-C₆₀ alkynyl group; a C₃-C₆₀ cycloalkyl group; a C₂-C₆₀ heterocycloalkyl group; a C₆-C₆₀ aryl group; a C₂-C₆₀ heteroaryl group; —SiRR′R″; —P(═O)RR′; a C₁-C₂₀ alkylamine group; a C₆-C₆₀ arylamine group; and a C₂-C₆₀ heteroarylamine group, substitution or unsubstitution with a substituent to which two or more of the above-mentioned substituents are bonded, or substitution or unsubstitution with a substituent linked with two or more substituents selected from among the above-mentioned substituents. For example, the “substituent linked with two or more substituents selected from among the substituents” may be a terphenyl group. In other words, the terphenyl group may be an aryl group, and may be interpreted as a substituent to which two phenyl groups are linked. The additional substituents may be further substituted. The substituents R, R′, and R″ may be the same as or different from each other, and each, independently, may be: hydrogen; deuterium; —CN; a substituted or unsubstituted C1-C60 alkyl group; a substituted or unsubstituted C3-C60 cycloalkyl group; a substituted or unsubstituted C6-C60 aryl group; or a substituted or unsubstituted C2-C60 heteroaryl group.

According to an embodiment of the present invention, the term “substituted or unsubstituted” refers to substitution or unsubstitution with one or more substituents selected from the group consisting of deuterium, a halogen group, —CN, SiRR′R″, P(═O)RR′, a C1 to C20 linear or branched alkyl group, a C6 to C60 aryl group, and a C2 to C60 heteroaryl group, where R, R′, and R″ may be the same as or different from each other, and each, independently, may be: hydrogen; deuterium; —CN; a C1-C60 alkyl group substituted or unsubstituted with deuterium, a halogen group, —CN, a C1 to C20 alkyl group, a C6 to C60 aryl group, or a C2-C60 heteroaryl group; a C3-C60 cycloalkyl group substituted or unsubstituted with deuterium, a halogen, —CN, a C1 to C20 alkyl group, a C6 to C60 aryl group, or a C2-C60 heteroaryl group; a C6-C60 aryl group substituted or unsubstituted with deuterium, a halogen, —CN, a C1 to C20 alkyl group, a C6 to C60 aryl group, or a C2-C60 heteroaryl group; or a C2-C60 heteroaryl group substituted or unsubstituted with deuterium, a halogen, —CN, a C1 to C20 alkyl group, a C6 to C60 aryl group, or a C2-C60 heteroaryl group.

The compound according to an embodiment of the present invention is characterized by being represented by Chemical Formula 1 above. More specifically, the compound represented by Chemical Formula 1 is characterized by having hydrogen; a substituted or unsubstituted N-containing heterocyclic group; a substituted or unsubstituted amine group; or —P(═O)RaRb bonded directly or via a linking group (L1) at the Z1 position of the core structure described above. With this characteristic, the compound of the present invention can be employed as a material of an organic material layer of an organic light emitting device.

The heterocyclic compound according to an embodiment of the present invention has excellent planar structure characteristics. Since the overlapping effect of molecules is improved due to this planarity, electron mobility is improved, thereby enabling the manufacture of low-voltage devices. When the compound of the present invention is employed in an electron transporting layer, an electron injecting layer, or a layer in which electron transport and electron injection are simultaneously performed, the electron mobility is improved, thus resulting in achievement of excellent low-voltage characteristics.

In an embodiment of the present invention, Z1 in Chemical Formula 1 is hydrogen; a monocyclic or polycyclic heterocyclic group which is substituted or unsubstituted and which contains one or more nitrogen (N) atoms; a substituted or unsubstituted amine group; or —P(═O)RaRb, wherein Ra and Rb are the same as or different from each other and each, independently, a substituted or unsubstituted aryl group; or a substituted or unsubstituted heteroaryl group.

In an embodiment of the present invention, Z1 in Chemical Formula 1 is substituted or unsubstituted, and is hydrogen; a monocyclic or polycyclic heterocyclic group which contains one or more nitrogen (N) atoms; a substituted or unsubstituted amine group; or —P(═O)RaRb, wherein Ra and Rb are the same as or different from each other and each, independently, a substituted or unsubstituted aryl group.

In an embodiment of the present invention, Z1 in Chemical Formula 1 is substituted or unsubstituted, and is hydrogen; a monocyclic or polycyclic heterocyclic group which contains one or more nitrogen (N) atoms; a substituted or unsubstituted amine group; or —P(═O)RaRb, wherein Ra and Rb are the same as or different from each other and each, independently, a substituted or unsubstituted C6-C20 aryl group.

In an embodiment of the present invention, Z1 in Chemical Formula 1 is substituted or unsubstituted, and is hydrogen; a monocyclic or polycyclic heterocyclic group which contains one or more nitrogen (N) atoms; a substituted or unsubstituted amine group; or —P(═O)RaRb, wherein Ra and Rb are the same as or different from each other and each, independently, a substituted or unsubstituted phenyl group.

In an embodiment of the present invention, Z1 in Chemical Formula 1 may be any one selected from hydrogen, or Chemical Formulas 2 to 9 below.

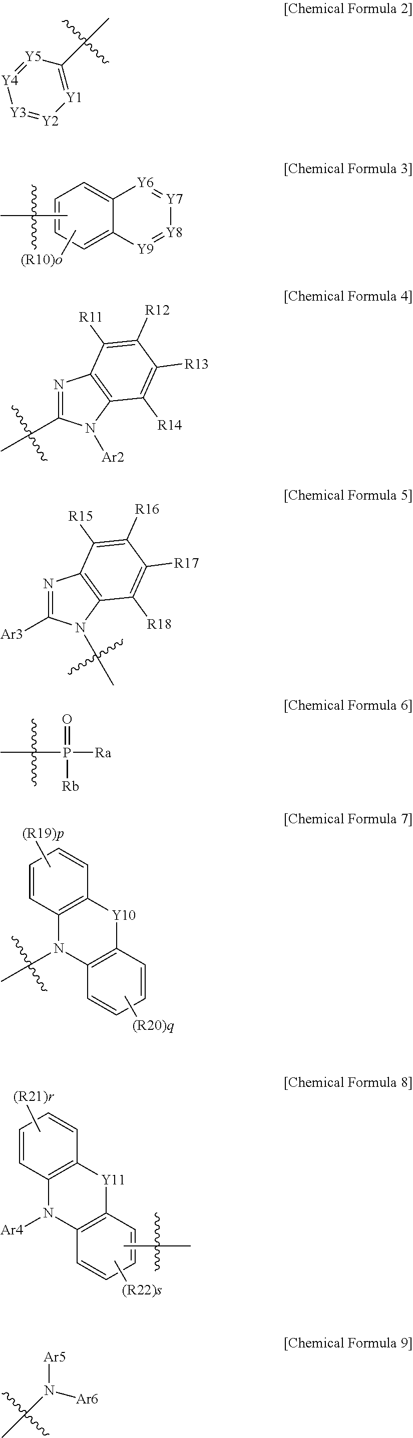

In Chemical Formulas 2 to 9, Y1 to Y9 are the same as or different from each other and each, independently, N or CRc; at least one from among Y1 to Y5 is N; at least one from among Y6 to Y9 is N; Y10 and Y11 are the same as or different from each other and each, independently, a direct bond, O, S, or CRdRe; Ar2 to Ar6, R10 to R22, and Rc to Re are the same as or different from each other and each, independently, selected from the group consisting of hydrogen, deuterium, a substituted or unsubstituted alkyl group, a substituted or unsubstituted aryl group, and a substituted or unsubstituted heteroaryl group, wherein two adjacent groups among these may be bonded to each other to form a substituted or unsubstituted aromatic hydrocarbon ring or a substituted or unsubstituted hetero ring; o is an integer of 1 to 3, and each R10 is the same as or different from each other when o is 2 or higher; p is an integer of 1 to 4, and each R19 is the same as or different from each other when p is 2 or higher; q is an integer of 1 to 4, and each R20 is the same as or different from each other when q is 2 or higher; r is an integer of 1 to 4, and each R21 is the same as or different from each other when r is 2 or higher; and s is an integer of 1 to 3, and each R22 is the same as or different from each other when s is 2 or higher.

In an embodiment of the present invention, Ar2 to Ar6, R10 to R22, and Rc to Re are the same as or different from each other, and each, independently, selected from the group consisting of hydrogen; a substituted or unsubstituted alkyl group; a substituted or unsubstituted aryl group; and a substituted or unsubstituted heteroaryl group, wherein two adjacent groups from among these may be bonded to each other to form a substituted or unsubstituted aromatic hydrocarbon ring or a substituted or unsubstituted hetero ring.

In an embodiment of the present invention, Ar2 to Ar6, R10 to R22, and Rc to Re are the same as or different from each other, and each, independently, selected from the group consisting of hydrogen; a substituted or unsubstituted C1-C4 alkyl group; a substituted or unsubstituted C6-C20 aryl group; and a substituted or unsubstituted C2-C30 heteroaryl group, wherein two adjacent groups from among these may be bonded to each other to form a substituted or unsubstituted aromatic hydrocarbon ring or a substituted or unsubstituted hetero ring.

In an embodiment of the present invention, Ar2 to Ar6, R10 to R22, and Rc to Re may be the same as or different from each other, and each, independently, may be a methyl group or an ethyl group.

In an embodiment of the present invention, Ar2 to Ar6, R10 to R22, and Rc to Re may be the same as or different from each other, and each, independently, may be a substituted or unsubstituted phenyl group; a substituted or unsubstituted biphenyl group; a substituted or unsubstituted terphenyl group; a substituted or unsubstituted naphthalene group; a substituted or unsubstituted anthracene group; a substituted or unsubstituted triphenyl group; a substituted or unsubstituted pyrene group; or a substituted or unsubstituted fluorene group.

In an embodiment of the present invention, Ar2 to Ar6, R10 to R22, and Rc to Re may be the same as or different from each other, and each, independently, may be a phenyl group; a biphenyl group; a terphenyl group; a naphthalene group; a anthracene group; a pyrene group; or a fluorene group substituted or unsubstituted with an alkyl group or an aryl group.

In an embodiment of the present invention, Ar2 to Ar6, R10 to R22, and Rc to Re may be the same as or different from each other, and each, independently, may be a phenyl group; a biphenyl group; a terphenyl group; a naphthalene group; a anthracene group; a pyrene group; or a fluorene group substituted or unsubstituted with a methyl group or a phenyl group.

In an embodiment of the present invention, Ar2 to Ar6, R10 to R22, and Rc to Re may be the same as or different from each other, and each, independently, may be a pyridine group; a pyrimidyl group; a carbazole group substituted or unsubstituted with an aryl group; a dibenzofuran group substituted or unsubstituted with an aryl group; or a dibenzothiophene group substituted or unsubstituted with an aryl group.

In an embodiment of the present invention, Ar2 to Ar6, R10 to R22, and Rc to Re may be the same as or different from each other, and each, independently, may be a pyridine group; a pyrimidyl group; a carbazole group substituted or unsubstituted with a C6-C20 aryl group; a dibenzofuran group substituted or unsubstituted with a C6-C20 aryl group; or a dibenzothiophene group substituted or unsubstituted with a C6-C20 aryl group.

In an embodiment of the present invention, Ar2 to Ar6, R10 to R22, and Rc to Re may be the same as or different from each other, and each, independently, may be a pyridine group; a pyrimidyl group; a carbazole group substituted or unsubstituted with a phenyl group, a biphenyl group, a terphenyl group, a naphthalene group, a triphenylene group or a pyrene group; a dibenzofuran group substituted or unsubstituted with a phenyl group; or a dibenzothiophene group substituted or unsubstituted with a phenyl group.

In an embodiment of the present invention, Ar2 to Ar6, R10 to R22, and Rc to Re may be the same as or different from each other, and each, independently, may be a substituent represented by the chemical formula below.

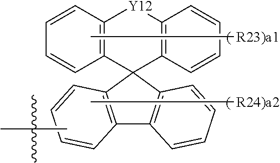

In the chemical formula above, Y12 is O, S, or NR25; R23 to R25 are the same as or different from each other, and each, independently, may be selected from the group consisting of hydrogen, a substituted or unsubstituted alkyl group, a substituted or unsubstituted aryl group, and a substituted or unsubstituted heteroaryl group; a1 is an integer of 1 to 8, and R23 is the same as or different from each other when a1 is 2 or more; and a2 is an integer of 1 to 7, and each R24 is the same as or different from each other when a2 is 2 or higher.

In an embodiment of the present invention, R23 to R25 are the same as or different from each other, and each, independently, may be hydrogen, or a substituted or unsubstituted C6-C20 aryl group.

In an embodiment of the present invention, R23 to R25 are the same as or different from each other, and each, independently, may be hydrogen, or a phenyl group.

In an embodiment of the present invention, Chemical Formula 2 above may be selected from the structural formulas below.

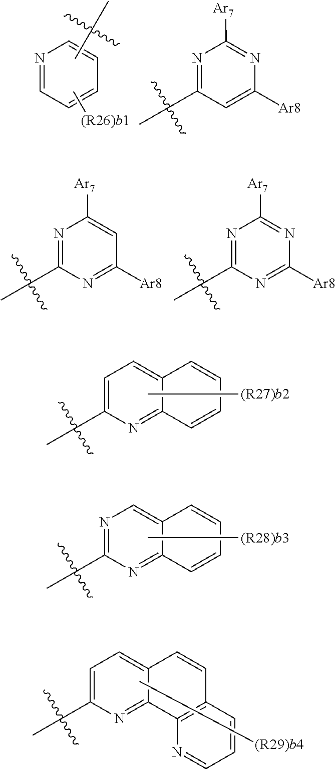

The substituents R26 to R29, Ar7, and Ar8 above are the same as those defined for the substituent Rc in Chemical Formula 1; b1 is an integer of 1 to 4, and each R26 is the same as or different from each other when b1 is 2 or higher; b2 is an integer of 1 to 6, and each R27 is the same as or different from each other when b2 is 2 or higher; b3 is an integer of 1 to 5, and each R28 is the same as or different from each other when b3 is 2 or higher; and b4 is an integer of 1 to 7, and each R29 is the same as or different from each other when b4 is 2 or higher.

In an embodiment of the present invention, Chemical Formula 3 may be represented by any one of the structural formulas below.

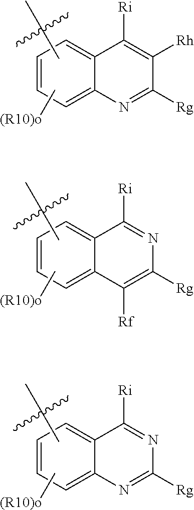

In the above structural formulas, Rf to Ri are the same as those defined for the substituent Rc in Chemical Formula 2, and o and R10 are the same as defined in Chemical Formula 3.

In an embodiment of the present invention, Ar1 may be represented by a C6-C20 aryl group or any one of the structural formulas below.

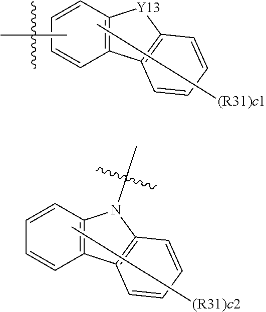

In the structural formulas above, Y13 is O, S, CRjRk or NRm; R31, R32, Rj, Rk, and Rm are the same as or different from each other, and each, independently, selected from the group consisting of hydrogen, deuterium, a substituted or unsubstituted alkyl group, a substituted or unsubstituted aryl group, and a substituted or unsubstituted heteroaryl group, where adjacent substituents may be bonded to each other to form a hydrocarbon ring or a heterocyclic group; cl is an integer of 1 to 7, and each R31 is the same as or different from each other when c1 is 2 or higher; and c2 is an integer of 1 to 8, and each R32 is the same as or different from each other when c2 is 2 or higher.

In an embodiment of the present invention, Ar1 is a substituted or unsubstituted phenyl group.

In an embodiment of the present invention, Y13 is O, S, CRjRk or Rm; and R31, R32, Rj, Rk and Rm are the same as or different from each other, and each, independently, selected from the group consisting of: hydrogen; a substituted or unsubstituted C1-C4 alkyl group; a substituted or unsubstituted C6 to C20 aryl group; and a substituted or unsubstituted C2-C30 heteroaryl group, where adjacent substituents may be bonded to each other to form a hydrocarbon ring or a heterocyclic group.

In the structural formulas above, Y13 is O, S, CRjRk or Rm; and R31, R32, Rj, Rk, and Rm are the same as or different from each other, and each, independently, selected from the group consisting of hydrogen, a methyl group, or a substituted or unsubstituted phenyl group, where adjacent substituents may be bonded to each other to form a hydrocarbon ring or a heterocyclic group.

In the structural formula above, Y13 is O, S, CRjRk or Rm; and R31, R32, Rj, Rk, and Rm are the same as or different from each other, and each, independently, selected from the group consisting of hydrogen a methyl group, or a phenyl group, where adjacent substituents may be bonded to each other to form a hydrocarbon ring or a heterocyclic group.

In an embodiment of the present invention, L1 is a direct bond; a substituted or unsubstituted arylene group; or a substituted or unsubstituted heteroarylene group.

In an embodiment of the present invention, L1 is a direct bond.

In an embodiment of the present invention, L1 is an arylene group substituted or unsubstituted with an aryl group or a heteroaryl group.

In an embodiment of the present invention, L1 is a C6-C20 arylene group substituted or unsubstituted with an aryl group or a heteroaryl group.

In an embodiment of the present invention, L1 is a phenylene group substituted or unsubstituted with a substituted or unsubstituted fluorene group; a biphenylene group; a terphenylene group; a naphthalene group; an anthracene group; a triphenylene group; or a pyrene group.

In an embodiment of the present invention, L1 is a phenylene group substituted or unsubstituted with one or more of a fluorene group substituted or unsubstituted with a C1-C4 alkyl group, and a carbazole group substituted or unsubstituted with a C6-C20 aryl group.

In an embodiment of the present invention, L1 is a phenylene group substituted or unsubstituted with one or more of a fluorene group substituted or unsubstituted with a methyl group, and a carbazole group substituted or unsubstituted with a phenyl group.

In an embodiment of the present invention, L1 is a biphenylene group; a terphenylene group; a naphthalene group; an anthracene group; a triphenylene group; or a pyrene group.

In an embodiment of the present invention, L1 is a C2-C30 heteroarylene group substituted or unsubstituted with an aryl group or a heteroaryl group.

In an embodiment of the present invention, L1 is a C2-C30 heteroarylene group substituted or unsubstituted with a C6-20 aryl group.

In an embodiment of the present invention, L1 is a dibenzofuran group; a dibenzothiophene group; a pyridyl group; or a carbazolene group substituted or unsubstituted with a phenyl group.

In an embodiment of the present invention, when L1 is a direct bond, Z1 may be a substituted or unsubstituted N-containing heterocyclic group; a substituted or unsubstituted amine group; or —P(═O)RaRb.

In an embodiment of the present invention, when L1 is a substituted or unsubstituted arylene group, Z1 may be hydrogen.

According to an embodiment of the present invention, Chemical Formula 1 above may be represented by any one of the following compounds, but is not limited thereto.

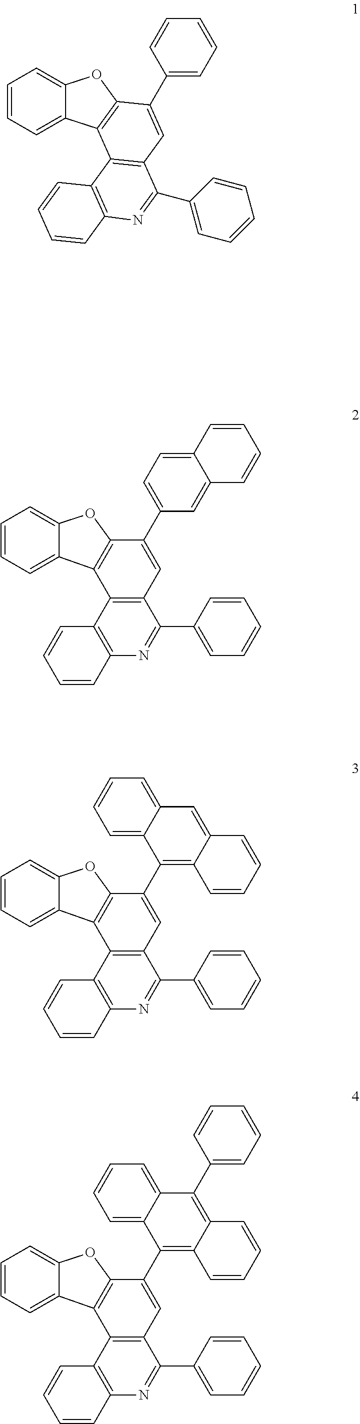 [SMILES: c1ccc(-c2nc3ccccc3c3c2cc(-c2ccccc2)c2oc4ccccc4c23)cc1 ; c1ccc(-c2nc3ccccc3c3c2cc(-c2ccc4ccccc4c2)c2oc4ccccc4c23)cc1 ; c1ccc(-c2nc3ccccc3c3c2cc(-c2c4ccccc4cc4ccccc24)c2oc4ccccc4c23)cc1 ; c1ccc(-c2c3ccccc3c(-c3cc4c(-c5ccccc5)nc5ccccc5c4c4c3oc3ccccc34)c3ccccc23)cc1]

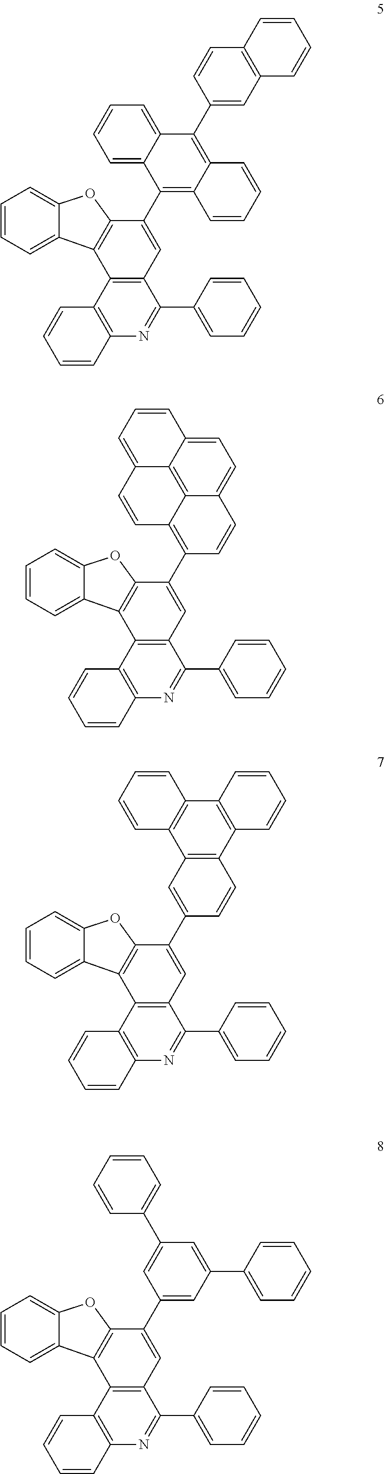 [SMILES: c1ccc(-c2nc3ccccc3c3c2cc(-c2c4ccccc4c(-c4ccc5ccccc5c4)c4ccccc24)c2oc4ccccc4c23)cc1 ; c1ccc(-c2nc3ccccc3c3c2cc(-c2ccc4ccc5cccc6ccc2c4c56)c2oc4ccccc4c23)cc1 ; c1ccc(-c2nc3ccccc3c3c2cc(-c2ccc4c5ccccc5c5ccccc5c4c2)c2oc4ccccc4c23)cc1 ; c1ccc(-c2cc(-c3ccccc3)cc(-c3cc4c(-c5ccccc5)nc5ccccc5c4c4c3oc3ccccc34)c2)cc1]

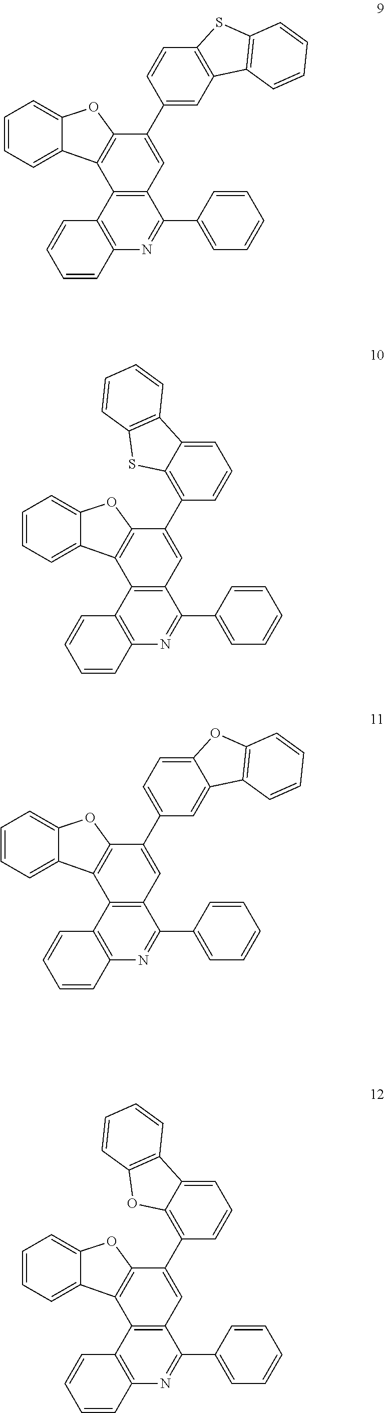 [SMILES: c1ccc(-c2nc3ccccc3c3c2cc(-c2ccc4sc5ccccc5c4c2)c2oc4ccccc4c23)cc1 ; c1ccc(-c2nc3ccccc3c3c2cc(-c2cccc4c2sc2ccccc24)c2oc4ccccc4c23)cc1 ; c1ccc(-c2nc3ccccc3c3c2cc(-c2ccc4oc5ccccc5c4c2)c2oc4ccccc4c23)cc1 ; c1ccc(-c2nc3ccccc3c3c2cc(-c2cccc4c2oc2ccccc24)c2oc4ccccc4c23)cc1]

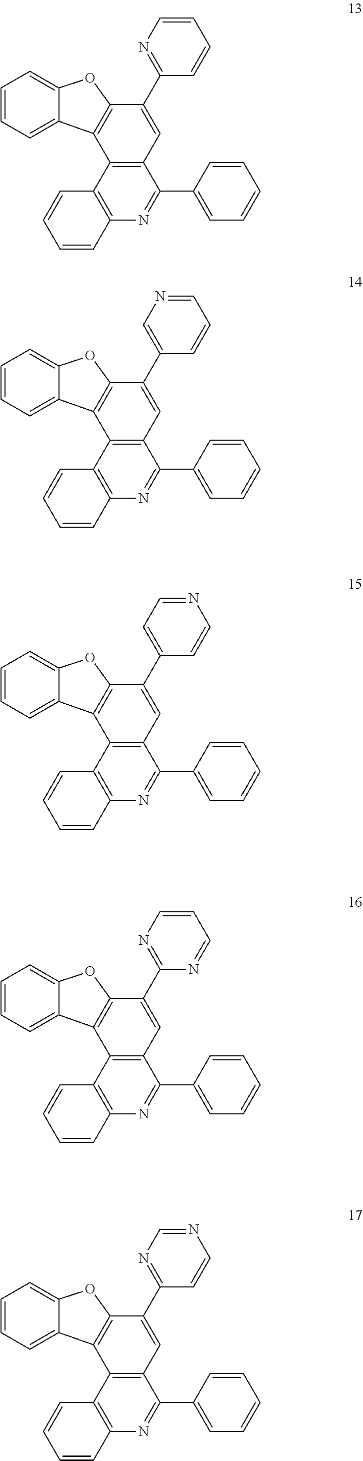 [SMILES: c1ccc(-c2nc3ccccc3c3c2cc(-c2ccccn2)c2oc4ccccc4c23)cc1 ; c1ccc(-c2nc3ccccc3c3c2cc(-c2cccnc2)c2oc4ccccc4c23)cc1 ; c1ccc(-c2nc3ccccc3c3c2cc(-c2ccncc2)c2oc4ccccc4c23)cc1 ; c1ccc(-c2nc3ccccc3c3c2cc(-c2ncccn2)c2oc4ccccc4c23)cc1 ; c1ccc(-c2nc3ccccc3c3c2cc(-c2ccncn2)c2oc4ccccc4c23)cc1]

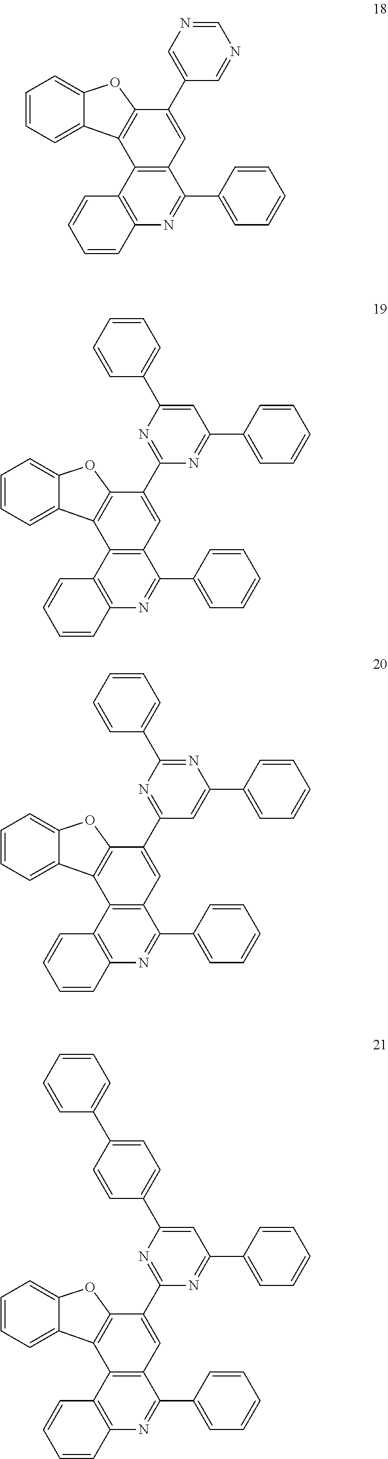

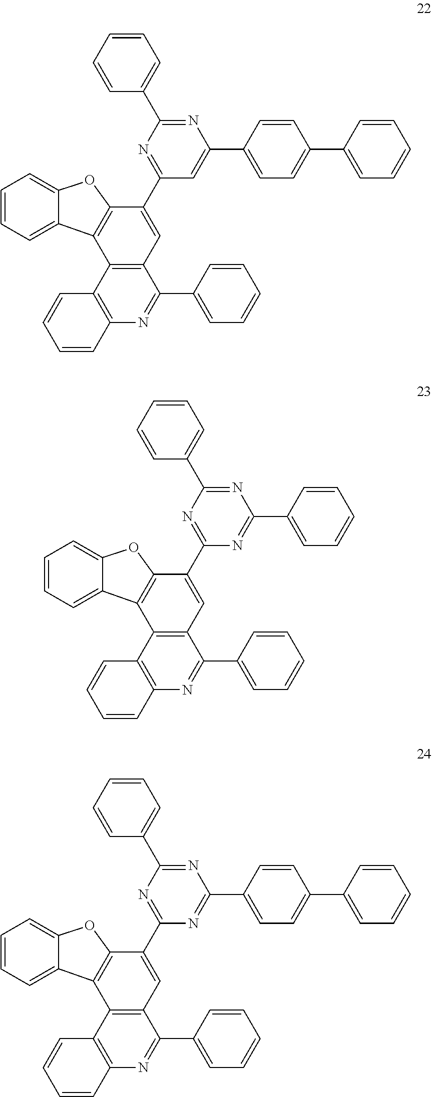 [SMILES: c1ccc(-c2ccc(-c3cc(-c4cc5c(-c6ccccc6)nc6ccccc6c5c5c4oc4ccccc45)nc(-c4ccccc4)n3)cc2)cc1 ; c1ccc(-c2nc(-c3ccccc3)nc(-c3cc4c(-c5ccccc5)nc5ccccc5c4c4c3oc3ccccc34)n2)cc1 ; c1ccc(-c2ccc(-c3nc(-c4ccccc4)nc(-c4cc5c(-c6ccccc6)nc6ccccc6c5c5c4oc4ccccc45)n3)cc2)cc1]

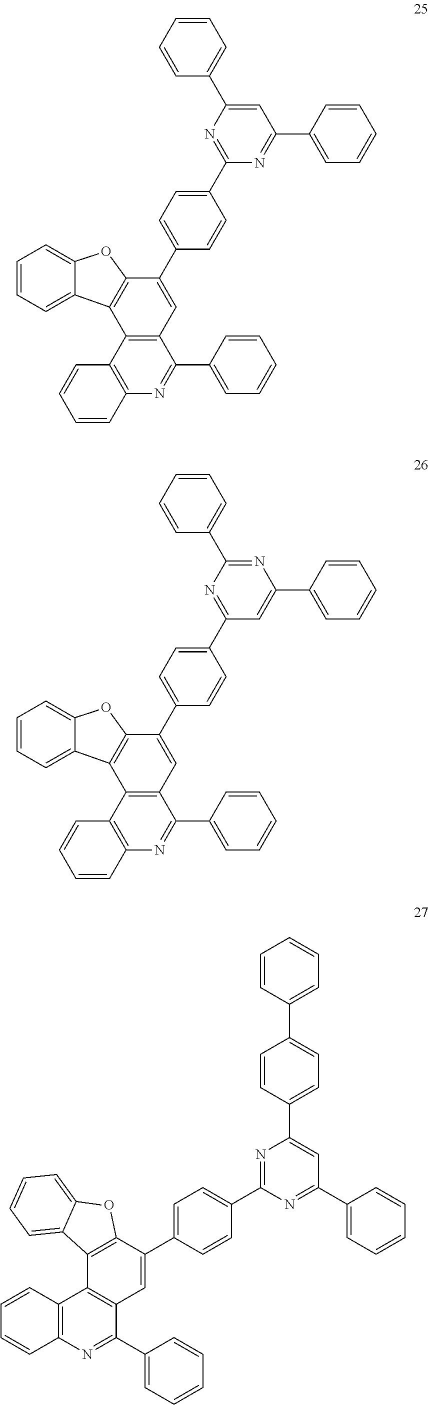 [SMILES: c1ccc(-c2cc(-c3ccccc3)nc(-c3ccc(-c4cc5c(-c6ccccc6)nc6ccccc6c5c5c4oc4ccccc45)cc3)n2)cc1 ; c1ccc(-c2cc(-c3ccc(-c4cc5c(-c6ccccc6)nc6ccccc6c5c5c4oc4ccccc45)cc3)nc(-c3ccccc3)n2)cc1 ; c1ccc(-c2ccc(-c3cc(-c4ccccc4)nc(-c4ccc(-c5cc6c(-c7ccccc7)nc7ccccc7c6c6c5oc5ccccc56)cc4)n3)cc2)cc1]

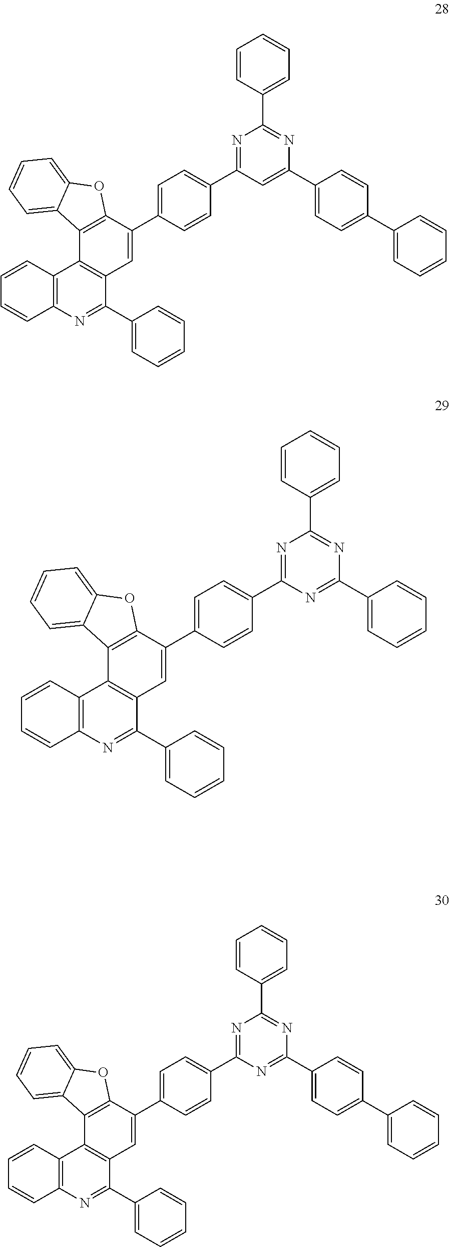 [SMILES: c1ccc(-c2ccc(-c3cc(-c4ccc(-c5cc6c(-c7ccccc7)nc7ccccc7c6c6c5oc5ccccc56)cc4)nc(-c4ccccc4)n3)cc2)cc1 ; c1ccc(-c2nc(-c3ccccc3)nc(-c3ccc(-c4cc5c(-c6ccccc6)nc6ccccc6c5c5c4oc4ccccc45)cc3)n2)cc1 ; c1ccc(-c2ccc(-c3nc(-c4ccccc4)nc(-c4ccc(-c5cc6c(-c7ccccc7)nc7ccccc7c6c6c5oc5ccccc56)cc4)n3)cc2)cc1]

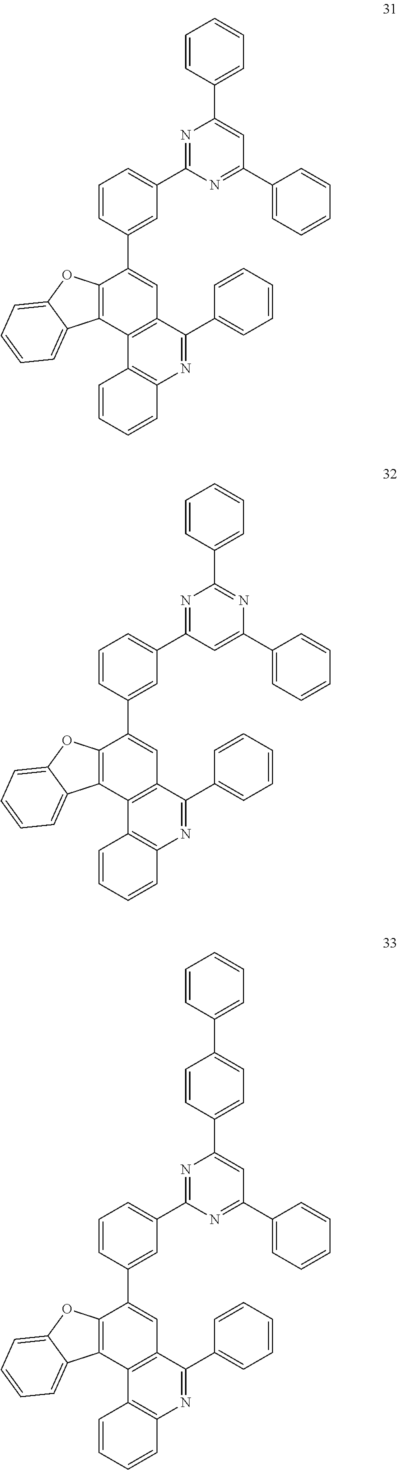 [SMILES: c1ccc(-c2cc(-c3ccccc3)nc(-c3cccc(-c4cc5c(-c6ccccc6)nc6ccccc6c5c5c4oc4ccccc45)c3)n2)cc1 ; c1ccc(-c2cc(-c3cccc(-c4cc5c(-c6ccccc6)nc6ccccc6c5c5c4oc4ccccc45)c3)nc(-c3ccccc3)n2)cc1 ; c1ccc(-c2ccc(-c3cc(-c4ccccc4)nc(-c4cccc(-c5cc6c(-c7ccccc7)nc7ccccc7c6c6c5oc5ccccc56)c4)n3)cc2)cc1]

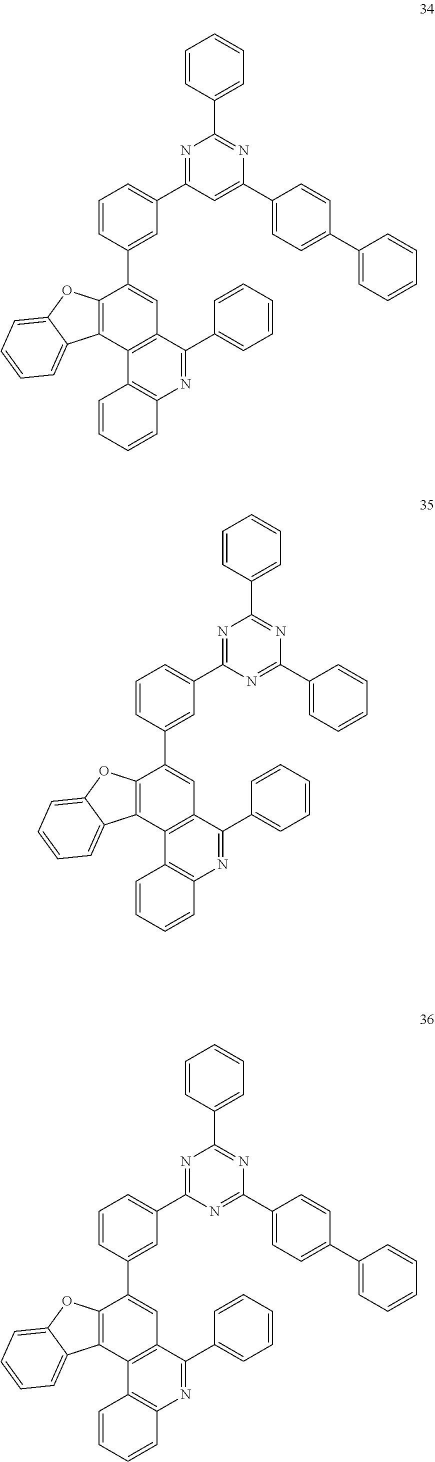 [SMILES: c1ccc(-c2ccc(-c3cc(-c4cccc(-c5cc6c(-c7ccccc7)nc7ccccc7c6c6c5oc5ccccc56)c4)nc(-c4ccccc4)n3)cc2)cc1 ; c1ccc(-c2nc(-c3ccccc3)nc(-c3cccc(-c4cc5c(-c6ccccc6)nc6ccccc6c5c5c4oc4ccccc45)c3)n2)cc1 ; c1ccc(-c2ccc(-c3nc(-c4ccccc4)nc(-c4cccc(-c5cc6c(-c7ccccc7)nc7ccccc7c6c6c5oc5ccccc56)c4)n3)cc2)cc1]

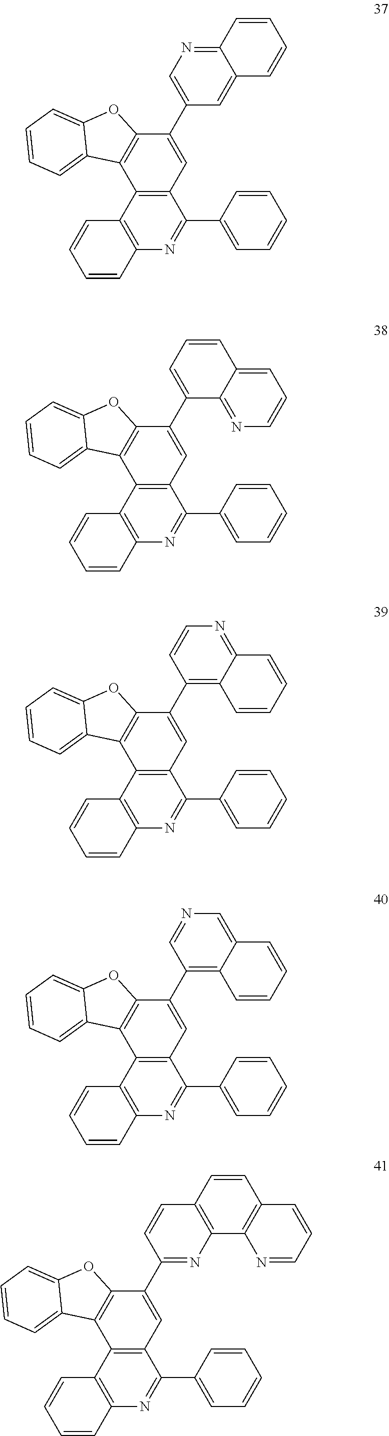 [SMILES: c1ccc(-c2nc3ccccc3c3c2cc(-c2cnc4ccccc4c2)c2oc4ccccc4c23)cc1 ; c1ccc(-c2nc3ccccc3c3c2cc(-c2cccc4cccnc24)c2oc4ccccc4c23)cc1 ; c1ccc(-c2nc3ccccc3c3c2cc(-c2ccnc4ccccc24)c2oc4ccccc4c23)cc1 ; c1ccc(-c2nc3ccccc3c3c2cc(-c2cncc4ccccc24)c2oc4ccccc4c23)cc1 ; c1ccc(-c2nc3ccccc3c3c2cc(-c2ccc4ccc5cccnc5c4n2)c2oc4ccccc4c23)cc1]

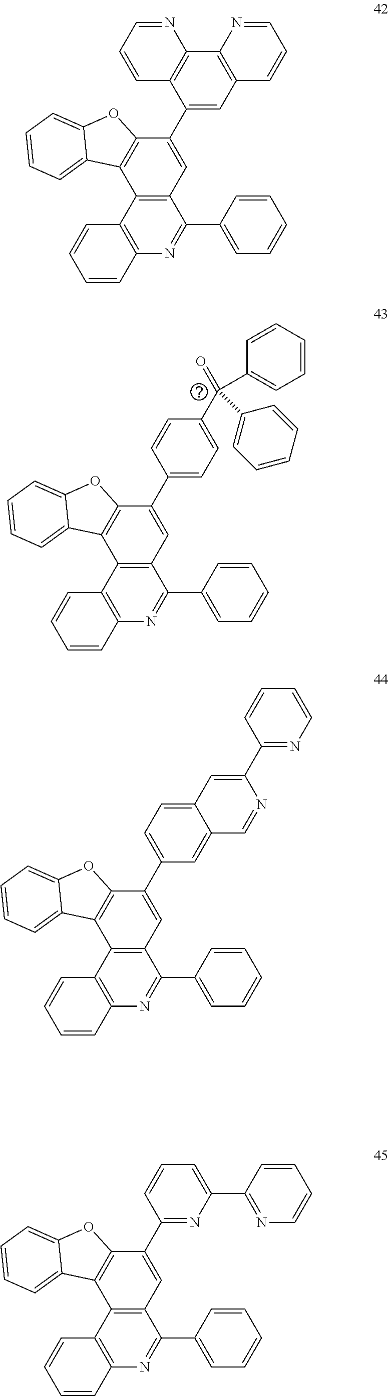

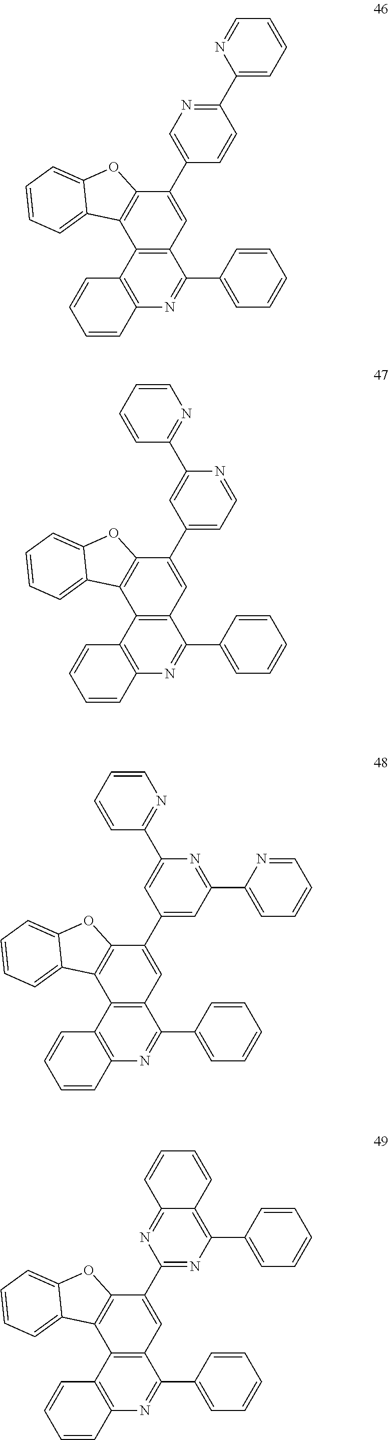 [SMILES: c1ccc(-c2nc3ccccc3c3c2cc(-c2ccc(-c4ccccn4)nc2)c2oc4ccccc4c23)cc1 ; c1ccc(-c2nc3ccccc3c3c2cc(-c2ccnc(-c4ccccn4)c2)c2oc4ccccc4c23)cc1 ; c1ccc(-c2nc3ccccc3c3c2cc(-c2cc(-c4ccccn4)nc(-c4ccccn4)c2)c2oc4ccccc4c23)cc1 ; c1ccc(-c2nc(-c3cc4c(-c5ccccc5)nc5ccccc5c4c4c3oc3ccccc34)nc3ccccc23)cc1]

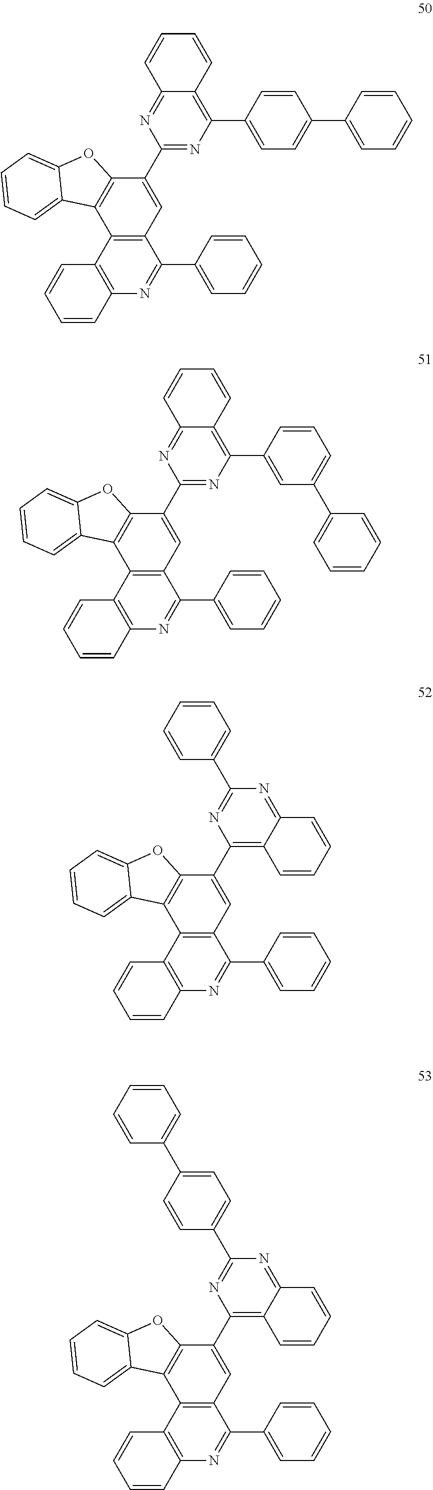 [SMILES: c1ccc(-c2ccc(-c3nc(-c4cc5c(-c6ccccc6)nc6ccccc6c5c5c4oc4ccccc45)nc4ccccc34)cc2)cc1 ; c1ccc(-c2cccc(-c3nc(-c4cc5c(-c6ccccc6)nc6ccccc6c5c5c4oc4ccccc45)nc4ccccc34)c2)cc1 ; c1ccc(-c2nc(-c3cc4c(-c5ccccc5)nc5ccccc5c4c4c3oc3ccccc34)c3ccccc3n2)cc1 ; c1ccc(-c2ccc(-c3nc(-c4cc5c(-c6ccccc6)nc6ccccc6c5c5c4oc4ccccc45)c4ccccc4n3)cc2)cc1]

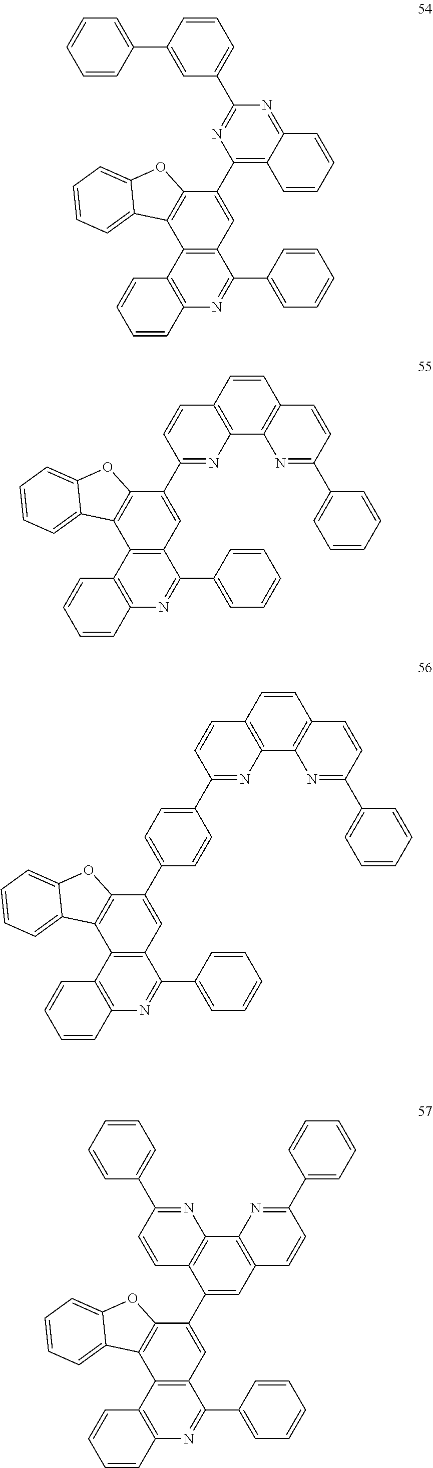 [SMILES: c1ccc(-c2cccc(-c3nc(-c4cc5c(-c6ccccc6)nc6ccccc6c5c5c4oc4ccccc45)c4ccccc4n3)c2)cc1 ; c1ccc(-c2ccc3ccc4ccc(-c5cc6c(-c7ccccc7)nc7ccccc7c6c6c5oc5ccccc56)nc4c3n2)cc1 ; c1ccc(-c2ccc3ccc4ccc(-c5ccc(-c6cc7c(-c8ccccc8)nc8ccccc8c7c7c6oc6ccccc67)cc5)nc4c3n2)cc1 ; c1ccc(-c2ccc3cc(-c4cc5c(-c6ccccc6)nc6ccccc6c5c5c4oc4ccccc45)c4ccc(-c5ccccc5)nc4c3n2)cc1]

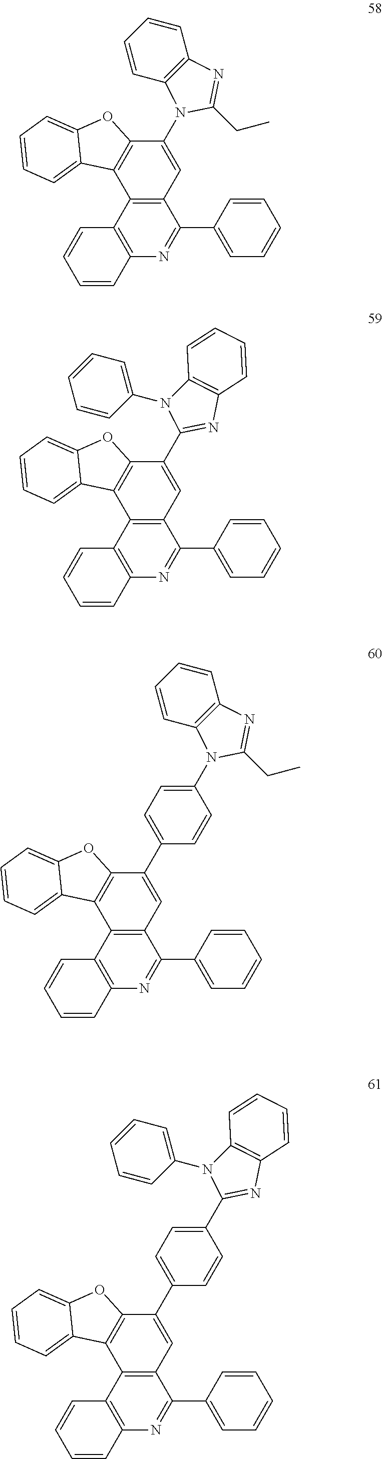 [SMILES: CCc1nc2ccccc2n1-c1cc2c(-c3ccccc3)nc3ccccc3c2c2c1oc1ccccc12 ; c1ccc(-c2nc3ccccc3c3c2cc(-c2nc4ccccc4n2-c2ccccc2)c2oc4ccccc4c23)cc1 ; CCc1nc2ccccc2n1-c1ccc(-c2cc3c(-c4ccccc4)nc4ccccc4c3c3c2oc2ccccc23)cc1 ; c1ccc(-c2nc3ccccc3c3c2cc(-c2ccc(-c4nc5ccccc5n4-c4ccccc4)cc2)c2oc4ccccc4c23)cc1]

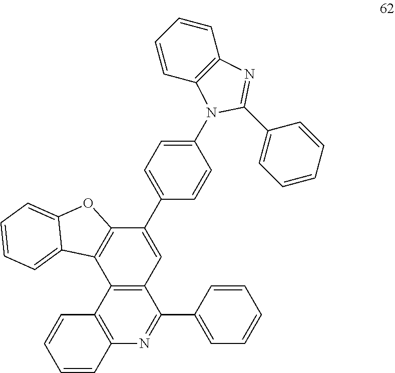 [SMILES: c1ccc(-c2nc3ccccc3c3c2cc(-c2ccc(-n4c(-c5ccccc5)nc5ccccc54)cc2)c2oc4ccccc4c23)cc1]

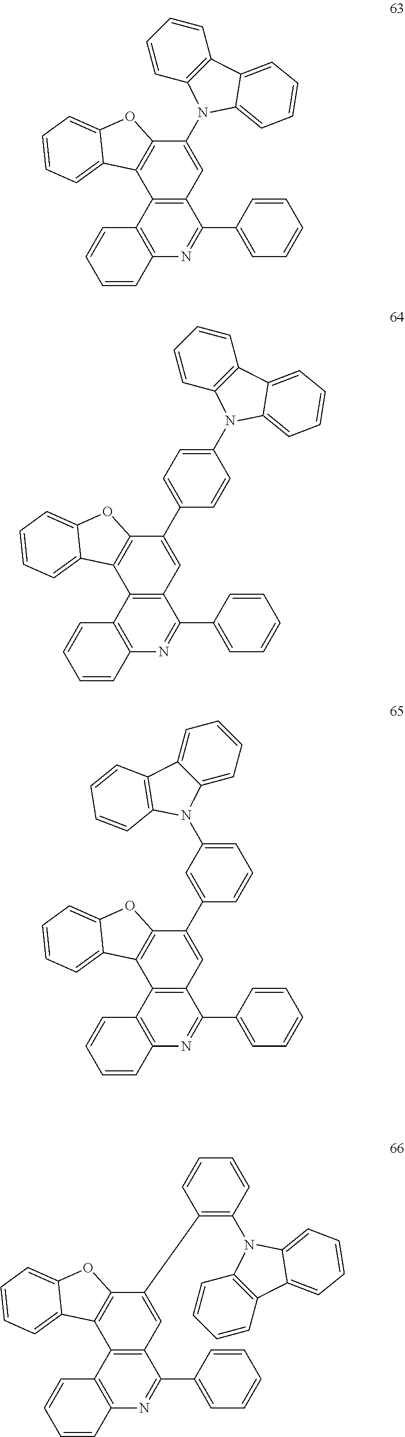 [SMILES: c1ccc(-c2nc3ccccc3c3c2cc(-n2c4ccccc4c4ccccc42)c2oc4ccccc4c23)cc1 ; c1ccc(-c2nc3ccccc3c3c2cc(-c2ccc(-n4c5ccccc5c5ccccc54)cc2)c2oc4ccccc4c23)cc1 ; c1ccc(-c2nc3ccccc3c3c2cc(-c2cccc(-n4c5ccccc5c5ccccc54)c2)c2oc4ccccc4c23)cc1 ; c1ccc(-c2nc3ccccc3c3c2cc(-c2ccccc2-n2c4ccccc4c4ccccc42)c2oc4ccccc4c23)cc1]

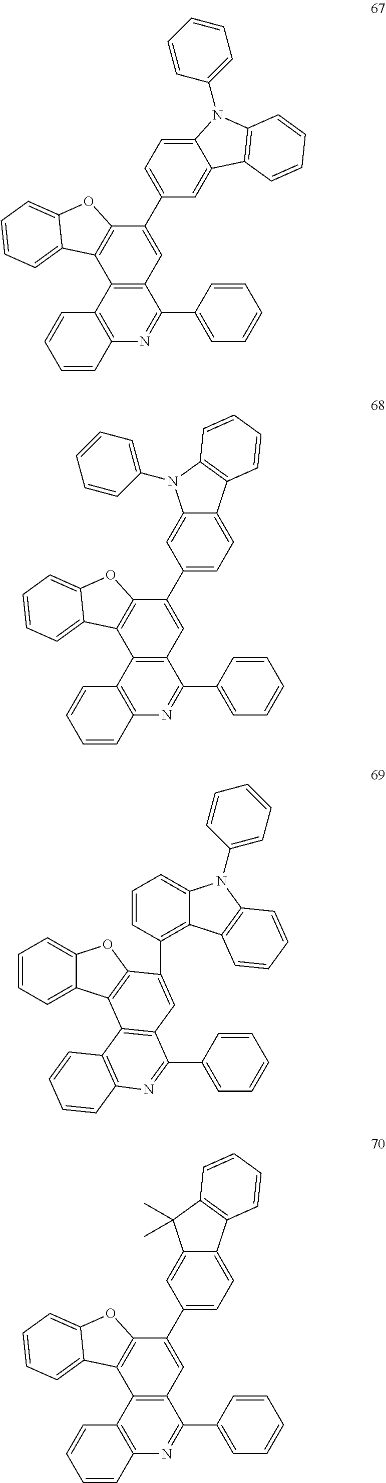 [SMILES: c1ccc(-c2nc3ccccc3c3c2cc(-c2ccc4c(c2)c2ccccc2n4-c2ccccc2)c2oc4ccccc4c23)cc1 ; c1ccc(-c2nc3ccccc3c3c2cc(-c2ccc4c5ccccc5n(-c5ccccc5)c4c2)c2oc4ccccc4c23)cc1 ; c1ccc(-c2nc3ccccc3c3c2cc(-c2cccc4c2c2ccccc2n4-c2ccccc2)c2oc4ccccc4c23)cc1 ; CC1(C)c2ccccc2-c2ccc(-c3cc4c(-c5ccccc5)nc5ccccc5c4c4c3oc3ccccc34)cc21]

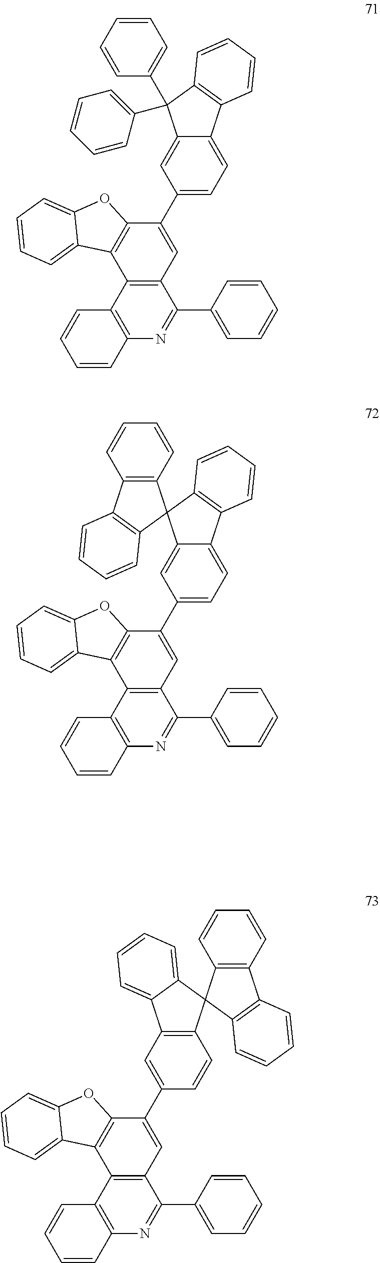 [SMILES: c1ccc(-c2nc3ccccc3c3c2cc(-c2ccc4c(c2)C(c2ccccc2)(c2ccccc2)c2ccccc2-4)c2oc4ccccc4c23)cc1 ; c1ccc(-c2nc3ccccc3c3c2cc(-c2ccc4c(c2)C2(c5ccccc5-c5ccccc52)c2ccccc2-4)c2oc4ccccc4c23)cc1 ; c1ccc(-c2nc3ccccc3c3c2cc(-c2ccc4c(c2)-c2ccccc2C42c4ccccc4-c4ccccc42)c2oc4ccccc4c23)cc1]

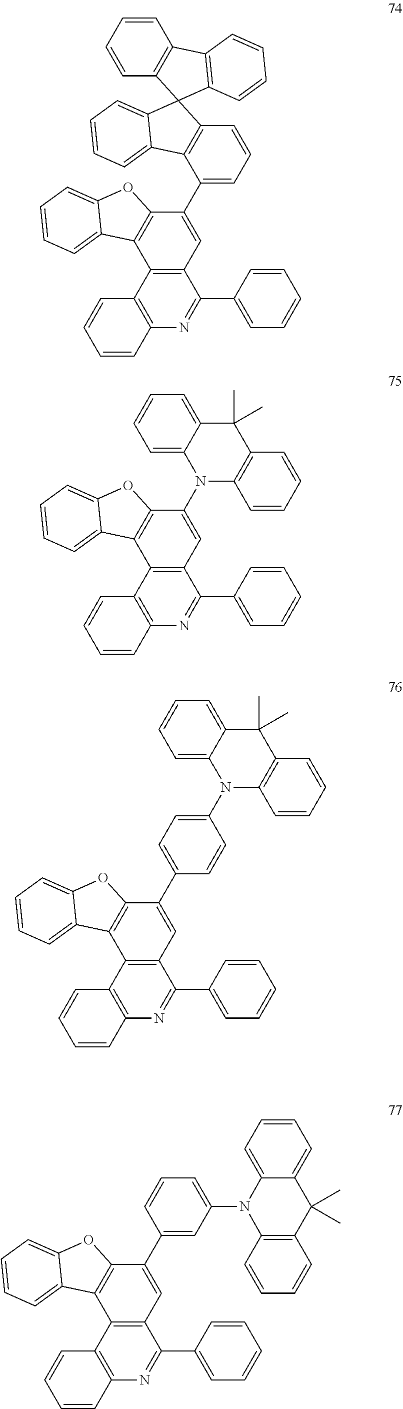

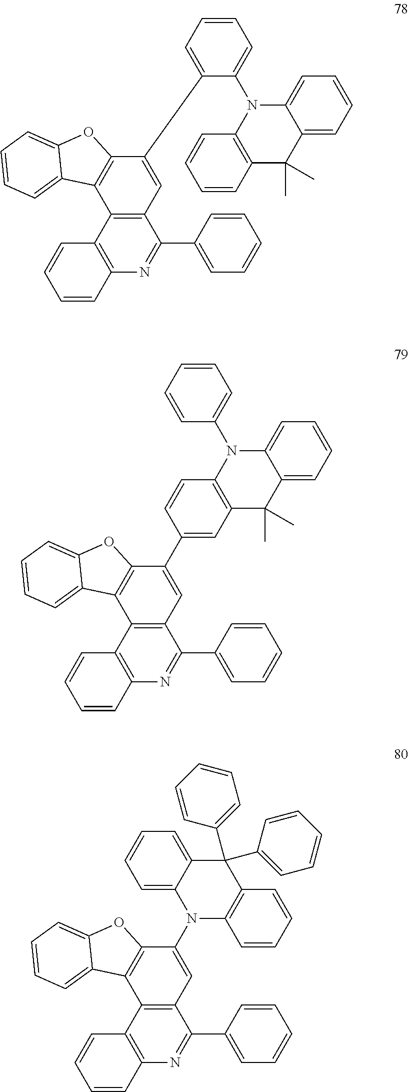 [SMILES: CC1(C)c2ccccc2N(c2ccccc2-c2cc3c(-c4ccccc4)nc4ccccc4c3c3c2oc2ccccc23)c2ccccc21 ; CC1(C)c2ccccc2N(c2ccccc2)c2ccc(-c3cc4c(-c5ccccc5)nc5ccccc5c4c4c3oc3ccccc34)cc21 ; c1ccc(-c2nc3ccccc3c3c2cc(N2c4ccccc4C(c4ccccc4)(c4ccccc4)c4ccccc42)c2oc4ccccc4c23)cc1]

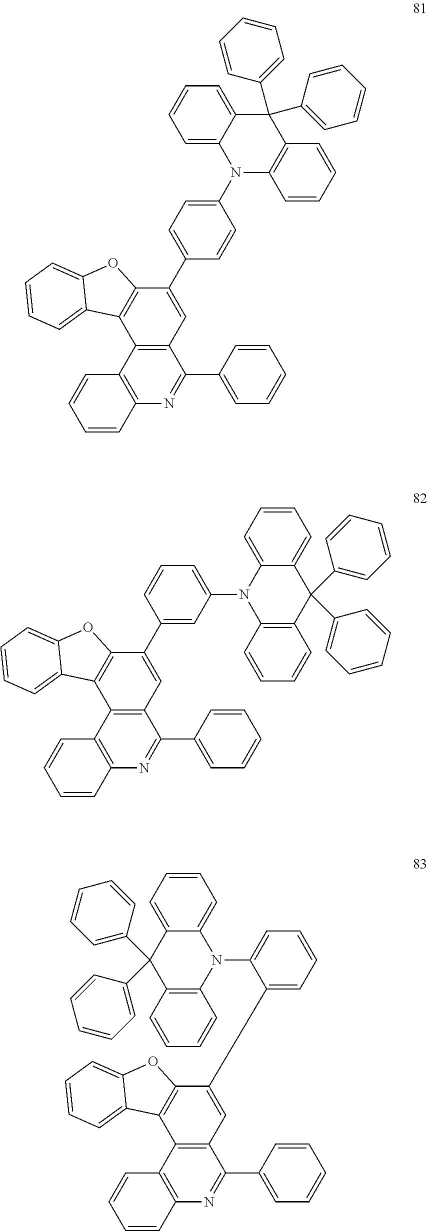 [SMILES: c1ccc(-c2nc3ccccc3c3c2cc(-c2ccc(N4c5ccccc5C(c5ccccc5)(c5ccccc5)c5ccccc54)cc2)c2oc4ccccc4c23)cc1 ; c1ccc(-c2nc3ccccc3c3c2cc(-c2cccc(N4c5ccccc5C(c5ccccc5)(c5ccccc5)c5ccccc54)c2)c2oc4ccccc4c23)cc1 ; c1ccc(-c2nc3ccccc3c3c2cc(-c2ccccc2N2c4ccccc4C(c4ccccc4)(c4ccccc4)c4ccccc42)c2oc4ccccc4c23)cc1]

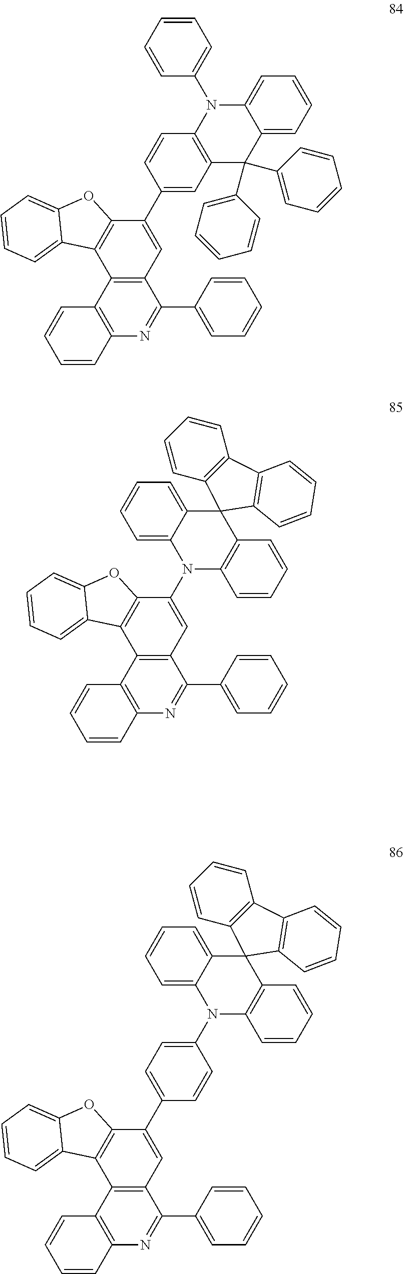 [SMILES: c1ccc(-c2nc3ccccc3c3c2cc(-c2ccc4c(c2)C(c2ccccc2)(c2ccccc2)c2ccccc2N4c2ccccc2)c2oc4ccccc4c23)cc1 ; c1ccc(-c2nc3ccccc3c3c2cc(N2c4ccccc4C4(c5ccccc5-c5ccccc54)c4ccccc42)c2oc4ccccc4c23)cc1 ; c1ccc(-c2nc3ccccc3c3c2cc(-c2ccc(N4c5ccccc5C5(c6ccccc6-c6ccccc65)c5ccccc54)cc2)c2oc4ccccc4c23)cc1]

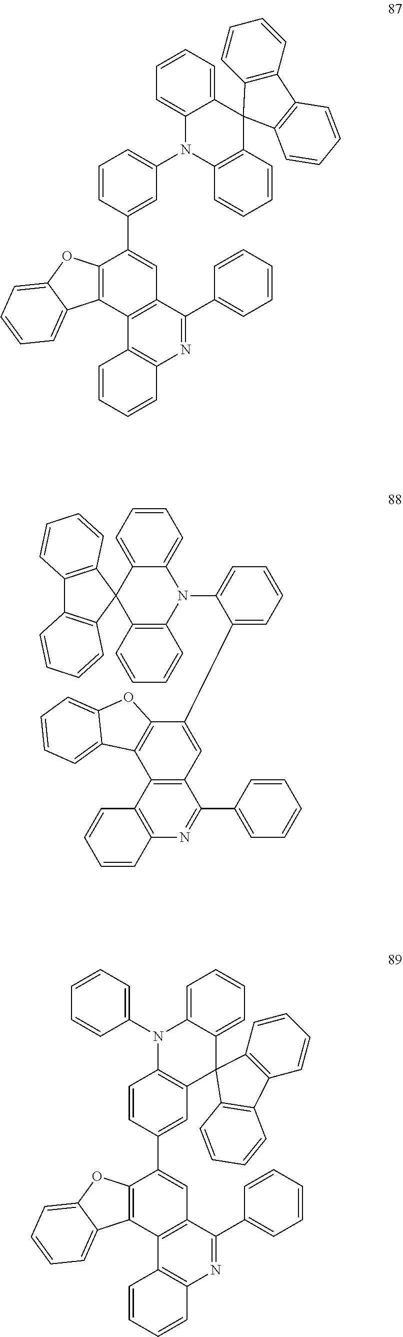 [SMILES: c1ccc(-c2nc3ccccc3c3c2cc(-c2cccc(N4c5ccccc5C5(c6ccccc6-c6ccccc65)c5ccccc54)c2)c2oc4ccccc4c23)cc1 ; c1ccc(-c2nc3ccccc3c3c2cc(-c2ccccc2N2c4ccccc4C4(c5ccccc5-c5ccccc54)c4ccccc42)c2oc4ccccc4c23)cc1 ; c1ccc(-c2nc3ccccc3c3c2cc(-c2ccc4c(c2)C2(c5ccccc5-c5ccccc52)c2ccccc2N4c2ccccc2)c2oc4ccccc4c23)cc1]

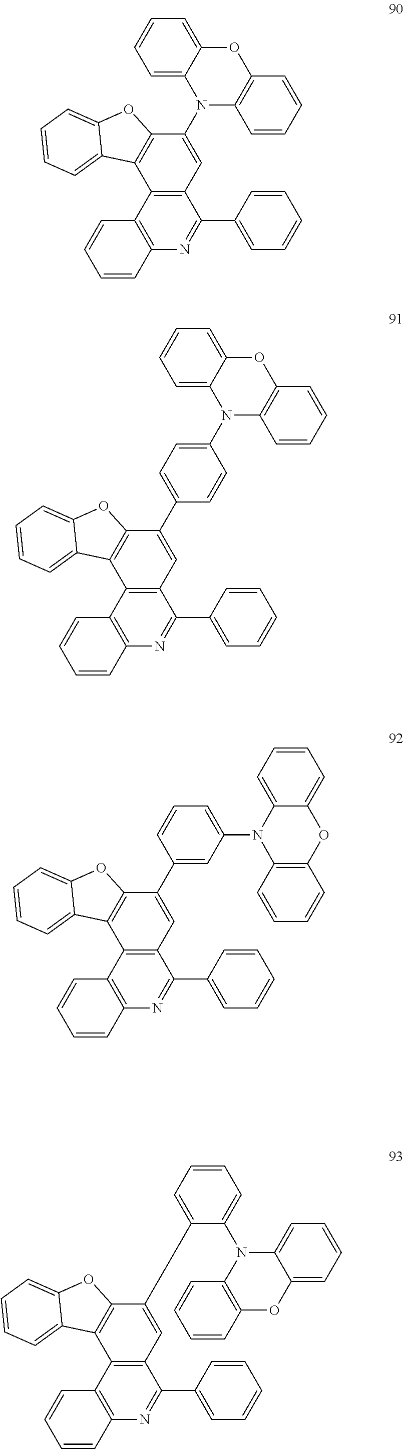 [SMILES: c1ccc(-c2nc3ccccc3c3c2cc(N2c4ccccc4Oc4ccccc42)c2oc4ccccc4c23)cc1 ; c1ccc(-c2nc3ccccc3c3c2cc(-c2ccc(N4c5ccccc5Oc5ccccc54)cc2)c2oc4ccccc4c23)cc1 ; c1ccc(-c2nc3ccccc3c3c2cc(-c2cccc(N4c5ccccc5Oc5ccccc54)c2)c2oc4ccccc4c23)cc1 ; c1ccc(-c2nc3ccccc3c3c2cc(-c2ccccc2N2c4ccccc4Oc4ccccc42)c2oc4ccccc4c23)cc1]

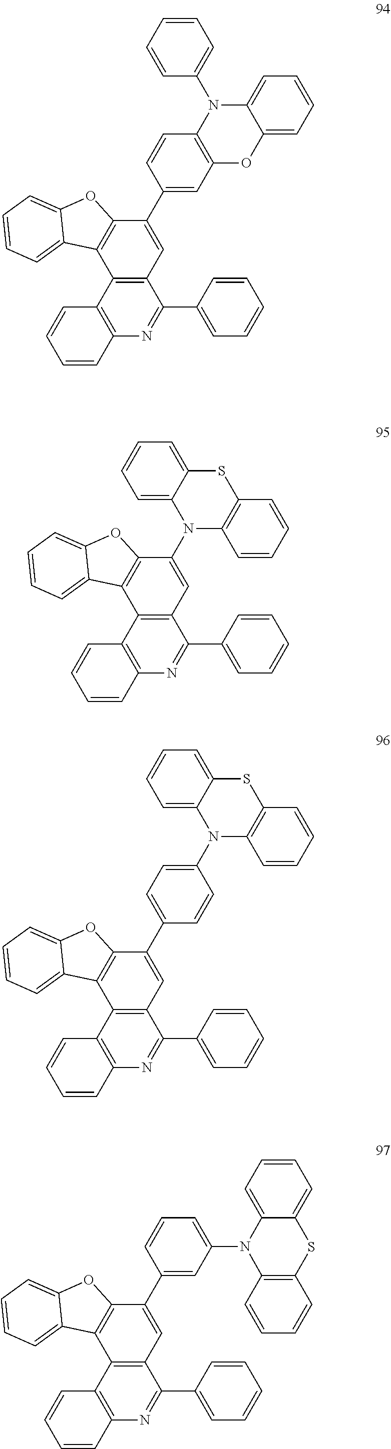 [SMILES: c1ccc(-c2nc3ccccc3c3c2cc(-c2ccc4c(c2)Oc2ccccc2N4c2ccccc2)c2oc4ccccc4c23)cc1 ; c1ccc(-c2nc3ccccc3c3c2cc(N2c4ccccc4Sc4ccccc42)c2oc4ccccc4c23)cc1 ; c1ccc(-c2nc3ccccc3c3c2cc(-c2ccc(N4c5ccccc5Sc5ccccc54)cc2)c2oc4ccccc4c23)cc1 ; c1ccc(-c2nc3ccccc3c3c2cc(-c2cccc(N4c5ccccc5Sc5ccccc54)c2)c2oc4ccccc4c23)cc1]

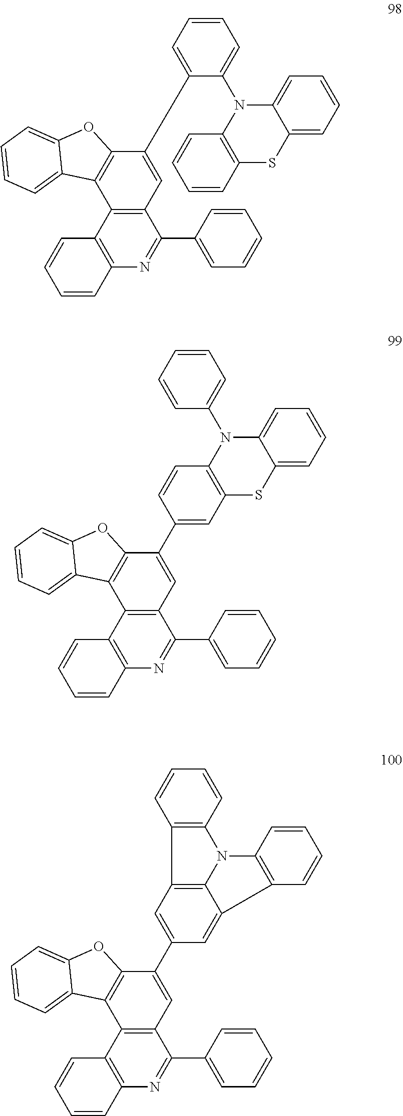 [SMILES: c1ccc(-c2nc3ccccc3c3c2cc(-c2ccccc2N2c4ccccc4Sc4ccccc42)c2oc4ccccc4c23)cc1 ; c1ccc(-c2nc3ccccc3c3c2cc(-c2ccc4c(c2)Sc2ccccc2N4c2ccccc2)c2oc4ccccc4c23)cc1 ; c1ccc(-c2nc3ccccc3c3c2cc(-c2cc4c5ccccc5n5c6ccccc6c(c2)c45)c2oc4ccccc4c23)cc1]

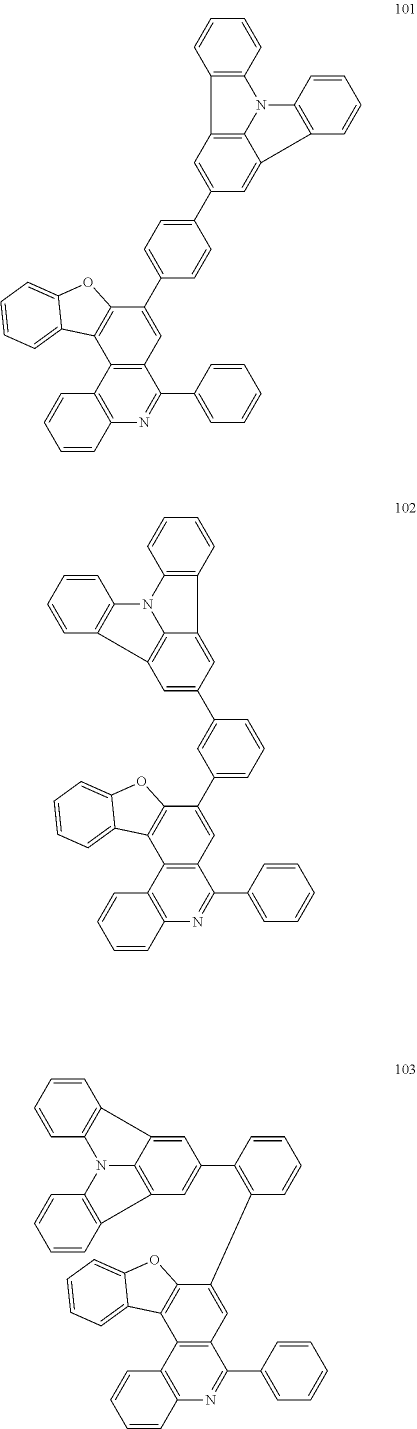 [SMILES: c1ccc(-c2nc3ccccc3c3c2cc(-c2ccc(-c4cc5c6ccccc6n6c7ccccc7c(c4)c56)cc2)c2oc4ccccc4c23)cc1 ; c1ccc(-c2nc3ccccc3c3c2cc(-c2cccc(-c4cc5c6ccccc6n6c7ccccc7c(c4)c56)c2)c2oc4ccccc4c23)cc1 ; c1ccc(-c2nc3ccccc3c3c2cc(-c2ccccc2-c2cc4c5ccccc5n5c6ccccc6c(c2)c45)c2oc4ccccc4c23)cc1]

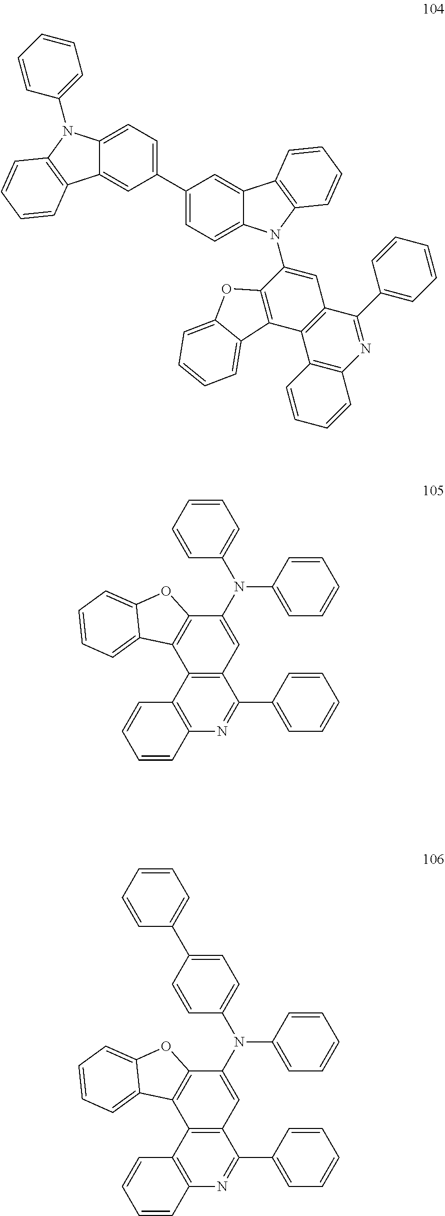 [SMILES: c1ccc(-c2nc3ccccc3c3c2cc(-n2c4ccccc4c4cc(-c5ccc6c(c5)c5ccccc5n6-c5ccccc5)ccc42)c2oc4ccccc4c23)cc1 ; c1ccc(-c2nc3ccccc3c3c2cc(N(c2ccccc2)c2ccccc2)c2oc4ccccc4c23)cc1 ; c1ccc(-c2ccc(N(c3ccccc3)c3cc4c(-c5ccccc5)nc5ccccc5c4c4c3oc3ccccc34)cc2)cc1]

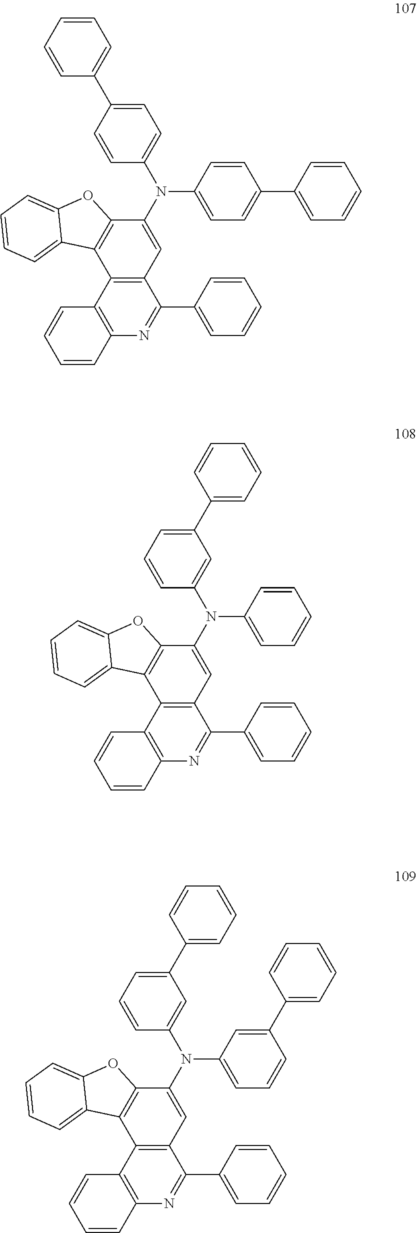 [SMILES: c1ccc(-c2ccc(N(c3ccc(-c4ccccc4)cc3)c3cc4c(-c5ccccc5)nc5ccccc5c4c4c3oc3ccccc34)cc2)cc1 ; c1ccc(-c2cccc(N(c3ccccc3)c3cc4c(-c5ccccc5)nc5ccccc5c4c4c3oc3ccccc34)c2)cc1 ; c1ccc(-c2cccc(N(c3cccc(-c4ccccc4)c3)c3cc4c(-c5ccccc5)nc5ccccc5c4c4c3oc3ccccc34)c2)cc1]

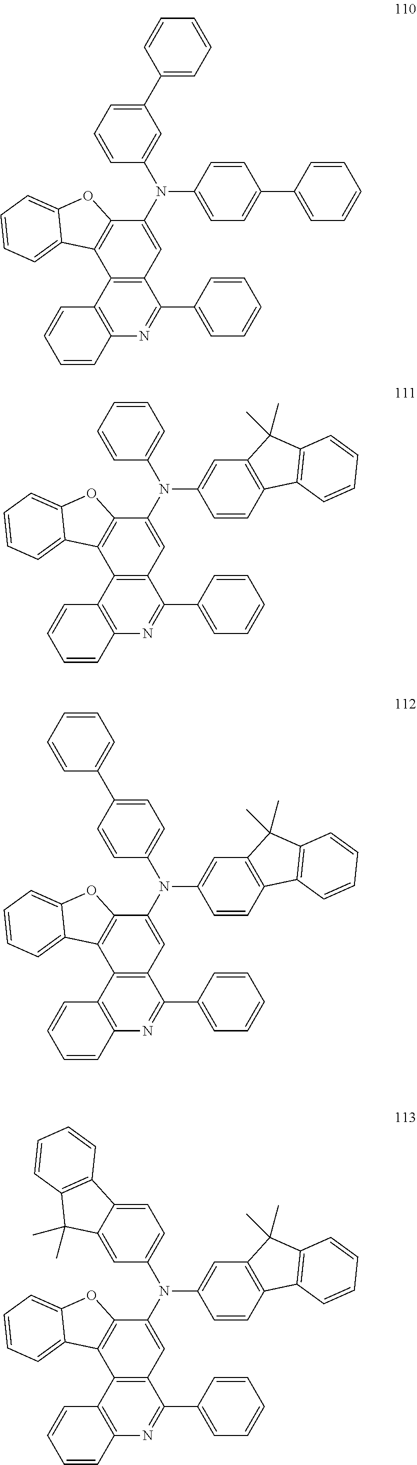

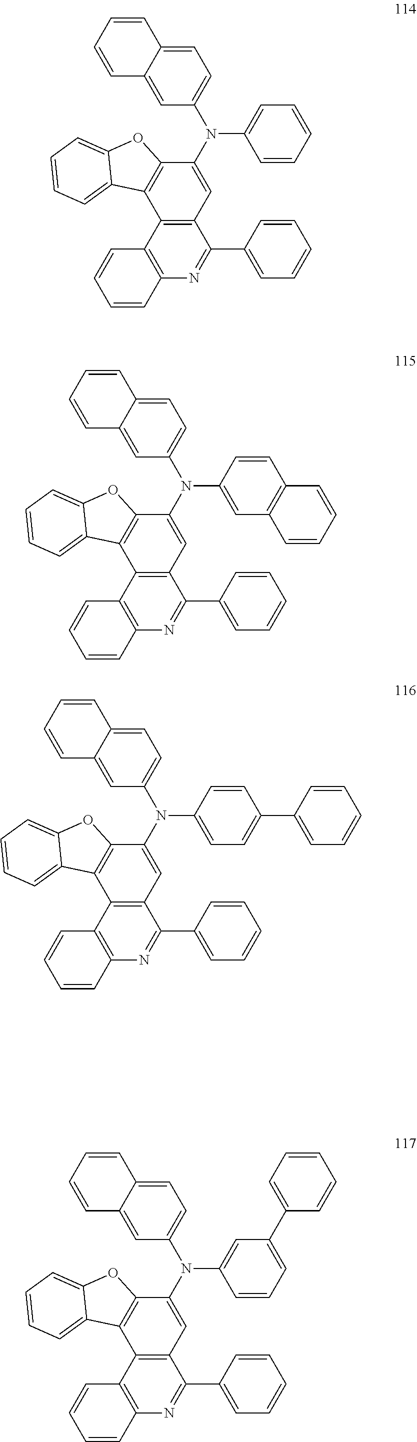

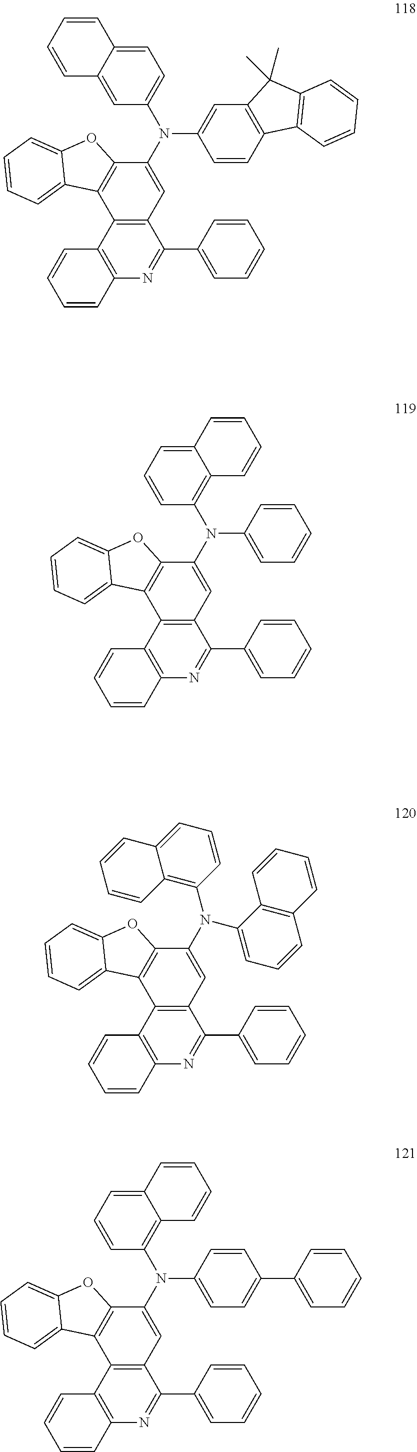 [SMILES: CC1(C)c2ccccc2-c2ccc(N(c3ccc4ccccc4c3)c3cc4c(-c5ccccc5)nc5ccccc5c4c4c3oc3ccccc34)cc21 ; c1ccc(-c2nc3ccccc3c3c2cc(N(c2ccccc2)c2cccc4ccccc24)c2oc4ccccc4c23)cc1 ; c1ccc(-c2nc3ccccc3c3c2cc(N(c2cccc4ccccc24)c2cccc4ccccc24)c2oc4ccccc4c23)cc1 ; c1ccc(-c2ccc(N(c3cccc4ccccc34)c3cc4c(-c5ccccc5)nc5ccccc5c4c4c3oc3ccccc34)cc2)cc1]

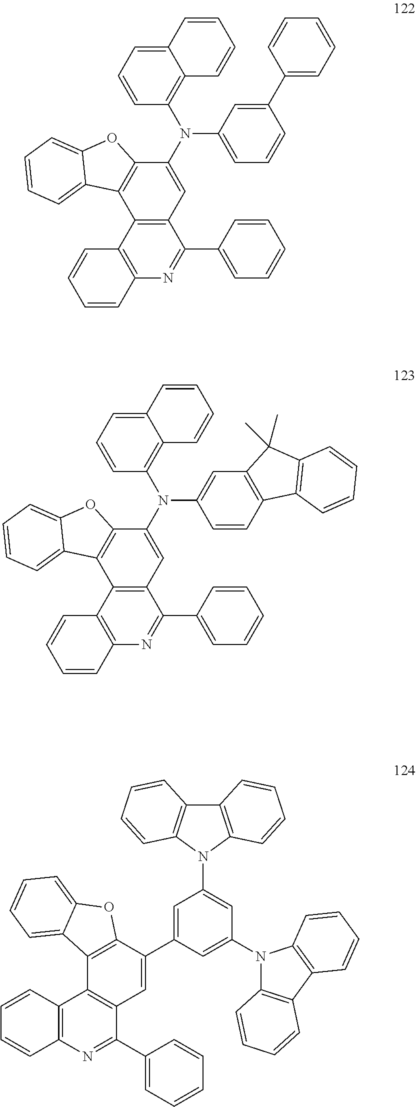 [SMILES: c1ccc(-c2cccc(N(c3cccc4ccccc34)c3cc4c(-c5ccccc5)nc5ccccc5c4c4c3oc3ccccc34)c2)cc1 ; CC1(C)c2ccccc2-c2ccc(N(c3cccc4ccccc34)c3cc4c(-c5ccccc5)nc5ccccc5c4c4c3oc3ccccc34)cc21 ; c1ccc(-c2nc3ccccc3c3c2cc(-c2cc(-n4c5ccccc5c5ccccc54)cc(-n4c5ccccc5c5ccccc54)c2)c2oc4ccccc4c23)cc1]

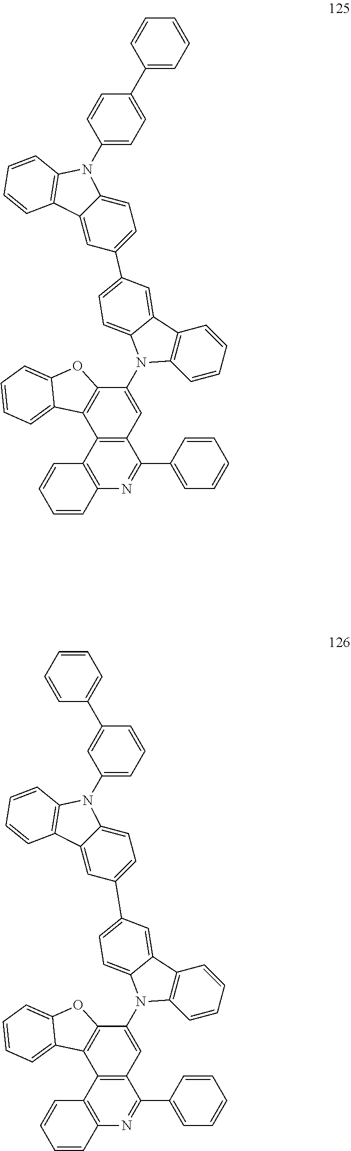 [SMILES: c1ccc(-c2ccc(-n3c4ccccc4c4cc(-c5ccc6c(c5)c5ccccc5n6-c5cc6c(-c7ccccc7)nc7ccccc7c6c6c5oc5ccccc56)ccc43)cc2)cc1 ; c1ccc(-c2cccc(-n3c4ccccc4c4cc(-c5ccc6c(c5)c5ccccc5n6-c5cc6c(-c7ccccc7)nc7ccccc7c6c6c5oc5ccccc56)ccc43)c2)cc1]

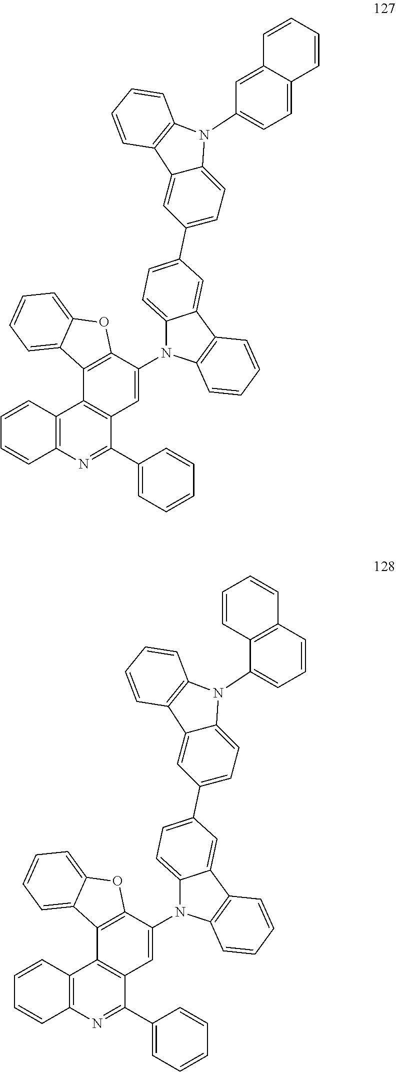 [SMILES: c1ccc(-c2nc3ccccc3c3c2cc(-n2c4ccccc4c4cc(-c5ccc6c(c5)c5ccccc5n6-c5ccc6ccccc6c5)ccc42)c2oc4ccccc4c23)cc1 ; c1ccc(-c2nc3ccccc3c3c2cc(-n2c4ccccc4c4cc(-c5ccc6c(c5)c5ccccc5n6-c5cccc6ccccc56)ccc42)c2oc4ccccc4c23)cc1]

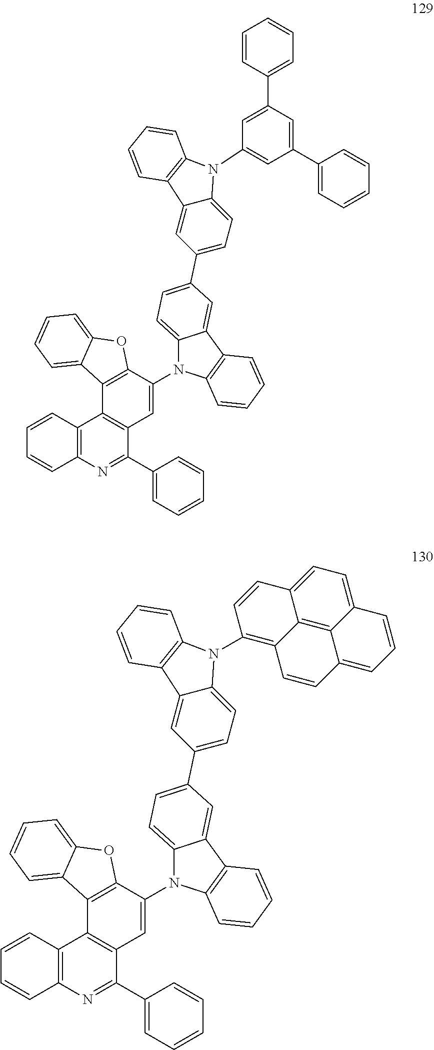 [SMILES: c1ccc(-c2cc(-c3ccccc3)cc(-n3c4ccccc4c4cc(-c5ccc6c(c5)c5ccccc5n6-c5cc6c(-c7ccccc7)nc7ccccc7c6c6c5oc5ccccc56)ccc43)c2)cc1 ; c1ccc(-c2nc3ccccc3c3c2cc(-n2c4ccccc4c4cc(-c5ccc6c(c5)c5ccccc5n6-c5ccc6ccc7cccc8ccc5c6c78)ccc42)c2oc4ccccc4c23)cc1]

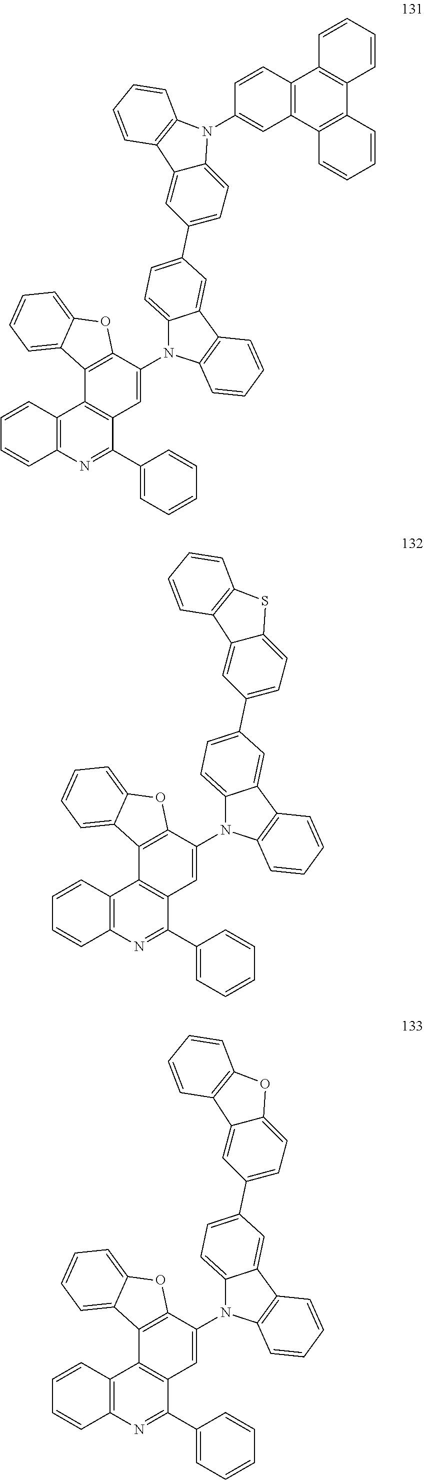

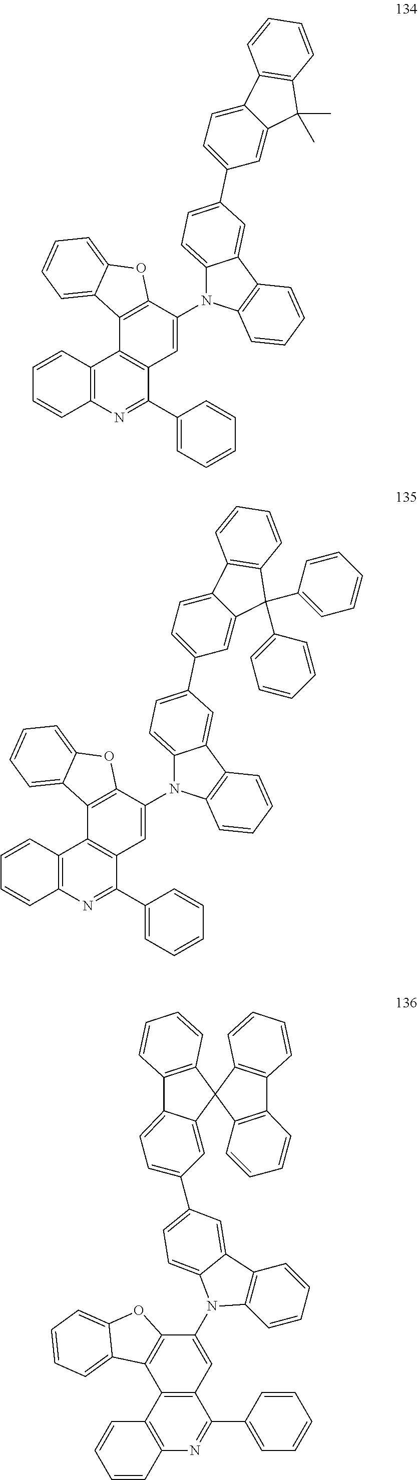 [SMILES: CC1(C)c2ccccc2-c2ccc(-c3ccc4c(c3)c3ccccc3n4-c3cc4c(-c5ccccc5)nc5ccccc5c4c4c3oc3ccccc34)cc21 ; c1ccc(-c2nc3ccccc3c3c2cc(-n2c4ccccc4c4cc(-c5ccc6c(c5)C(c5ccccc5)(c5ccccc5)c5ccccc5-6)ccc42)c2oc4ccccc4c23)cc1 ; c1ccc(-c2nc3ccccc3c3c2cc(-n2c4ccccc4c4cc(-c5ccc6c(c5)C5(c7ccccc7-c7ccccc75)c5ccccc5-6)ccc42)c2oc4ccccc4c23)cc1]

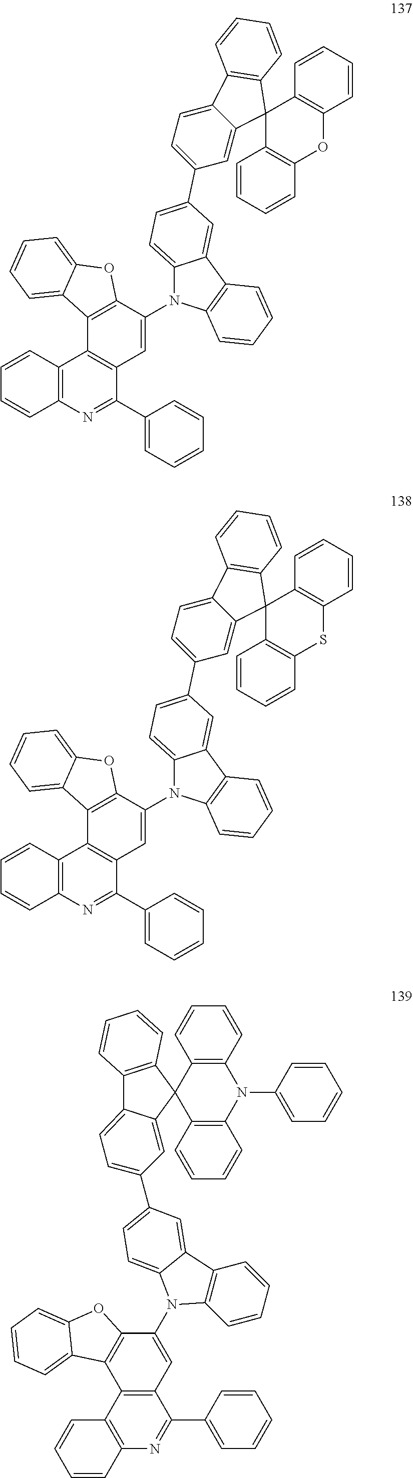 [SMILES: c1ccc(-c2nc3ccccc3c3c2cc(-n2c4ccccc4c4cc(-c5ccc6c(c5)C5(c7ccccc7Oc7ccccc75)c5ccccc5-6)ccc42)c2oc4ccccc4c23)cc1 ; c1ccc(-c2nc3ccccc3c3c2cc(-n2c4ccccc4c4cc(-c5ccc6c(c5)C5(c7ccccc7Sc7ccccc75)c5ccccc5-6)ccc42)c2oc4ccccc4c23)cc1 ; c1ccc(-c2nc3ccccc3c3c2cc(-n2c4ccccc4c4cc(-c5ccc6c(c5)C5(c7ccccc7-6)c6ccccc6N(c6ccccc6)c6ccccc65)ccc42)c2oc4ccccc4c23)cc1]

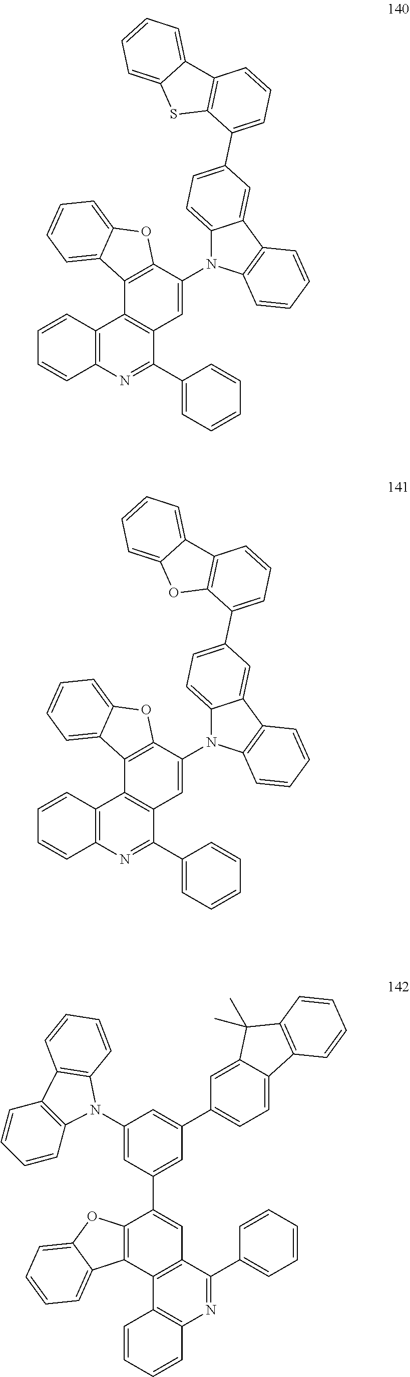 [SMILES: c1ccc(-c2nc3ccccc3c3c2cc(-n2c4ccccc4c4cc(-c5cccc6c5sc5ccccc56)ccc42)c2oc4ccccc4c23)cc1 ; c1ccc(-c2nc3ccccc3c3c2cc(-n2c4ccccc4c4cc(-c5cccc6c5oc5ccccc56)ccc42)c2oc4ccccc4c23)cc1 ; CC1(C)c2ccccc2-c2ccc(-c3cc(-c4cc5c(-c6ccccc6)nc6ccccc6c5c5c4oc4ccccc45)cc(-n4c5ccccc5c5ccccc54)c3)cc21]

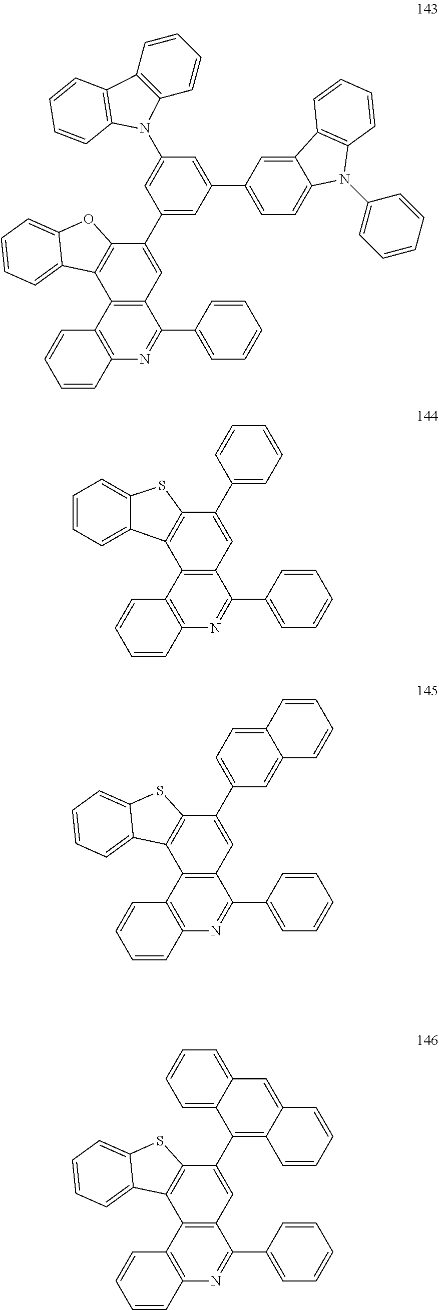 [SMILES: c1ccc(-c2nc3ccccc3c3c2cc(-c2cc(-c4ccc5c(c4)c4ccccc4n5-c4ccccc4)cc(-n4c5ccccc5c5ccccc54)c2)c2oc4ccccc4c23)cc1 ; c1ccc(-c2nc3ccccc3c3c2cc(-c2ccccc2)c2sc4ccccc4c23)cc1 ; c1ccc(-c2nc3ccccc3c3c2cc(-c2ccc4ccccc4c2)c2sc4ccccc4c23)cc1 ; c1ccc(-c2nc3ccccc3c3c2cc(-c2c4ccccc4cc4ccccc24)c2sc4ccccc4c23)cc1]

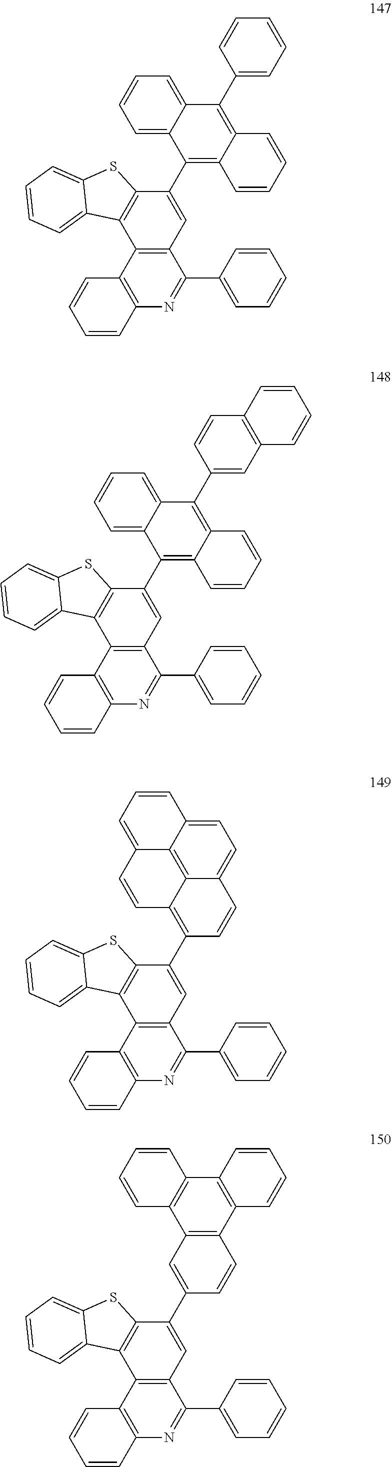

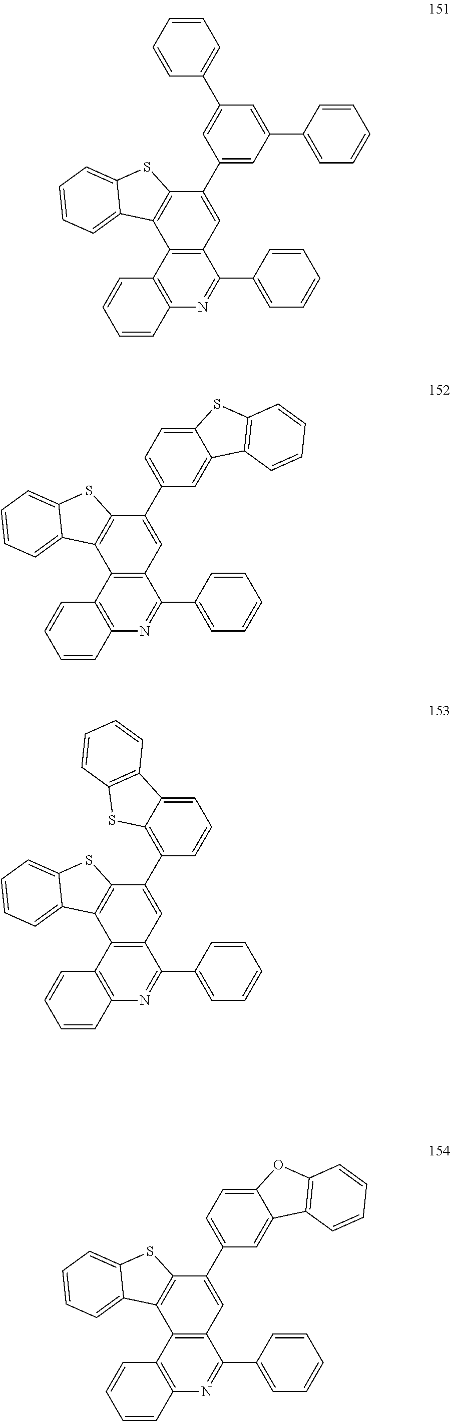 [SMILES: c1ccc(-c2cc(-c3ccccc3)cc(-c3cc4c(-c5ccccc5)nc5ccccc5c4c4c3sc3ccccc34)c2)cc1 ; c1ccc(-c2nc3ccccc3c3c2cc(-c2ccc4sc5ccccc5c4c2)c2sc4ccccc4c23)cc1 ; c1ccc(-c2nc3ccccc3c3c2cc(-c2cccc4c2sc2ccccc24)c2sc4ccccc4c23)cc1 ; c1ccc(-c2nc3ccccc3c3c2cc(-c2ccc4oc5ccccc5c4c2)c2sc4ccccc4c23)cc1]

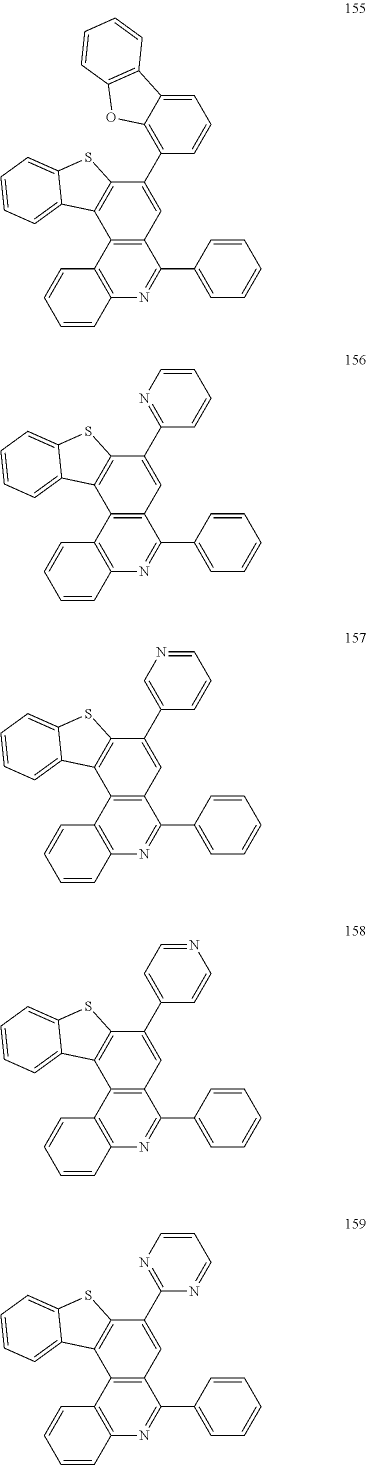 [SMILES: c1ccc(-c2nc3ccccc3c3c2cc(-c2cccc4c2oc2ccccc24)c2sc4ccccc4c23)cc1 ; c1ccc(-c2nc3ccccc3c3c2cc(-c2ccccn2)c2sc4ccccc4c23)cc1 ; c1ccc(-c2nc3ccccc3c3c2cc(-c2cccnc2)c2sc4ccccc4c23)cc1 ; c1ccc(-c2nc3ccccc3c3c2cc(-c2ccncc2)c2sc4ccccc4c23)cc1 ; c1ccc(-c2nc3ccccc3c3c2cc(-c2ncccn2)c2sc4ccccc4c23)cc1]

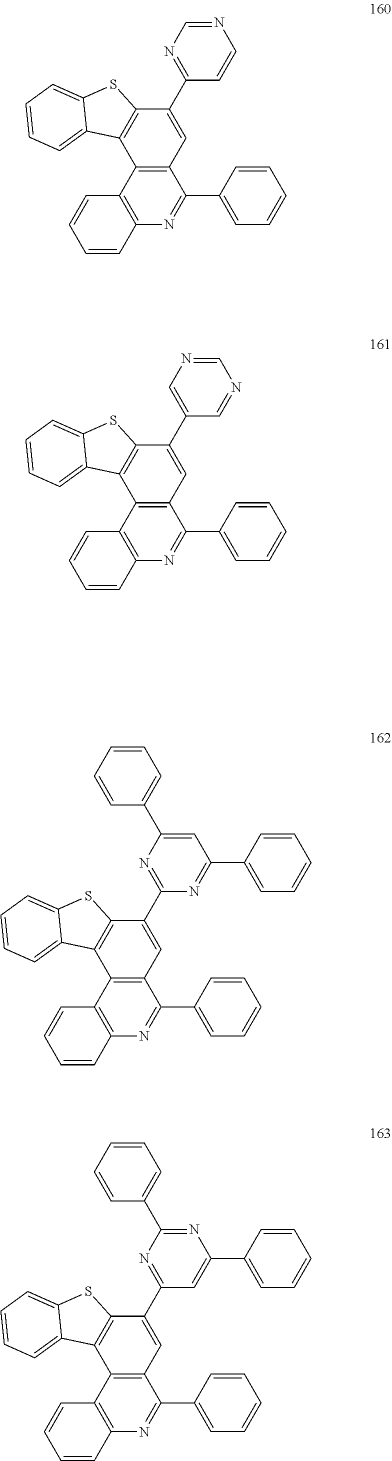 [SMILES: c1ccc(-c2nc3ccccc3c3c2cc(-c2ccncn2)c2sc4ccccc4c23)cc1 ; c1ccc(-c2nc3ccccc3c3c2cc(-c2cncnc2)c2sc4ccccc4c23)cc1 ; c1ccc(-c2cc(-c3ccccc3)nc(-c3cc4c(-c5ccccc5)nc5ccccc5c4c4c3sc3ccccc34)n2)cc1 ; c1ccc(-c2cc(-c3cc4c(-c5ccccc5)nc5ccccc5c4c4c3sc3ccccc34)nc(-c3ccccc3)n2)cc1]

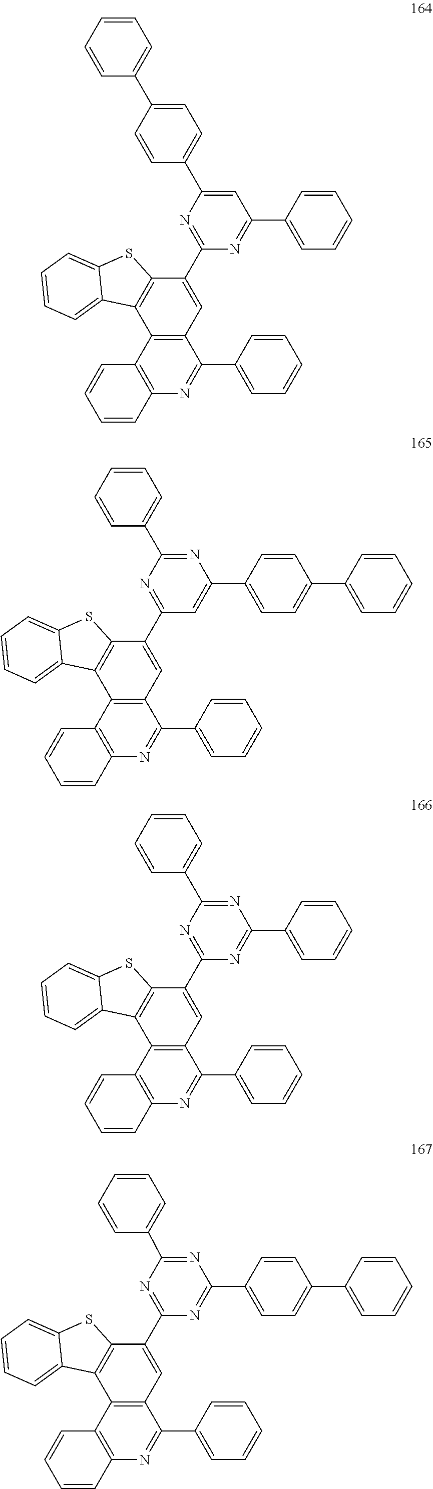

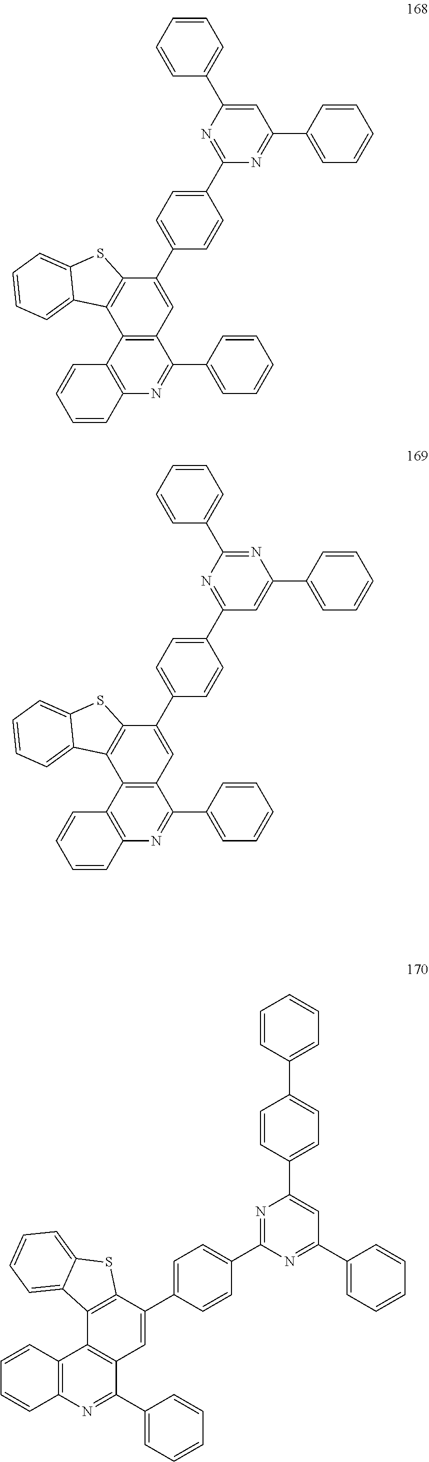 [SMILES: c1ccc(-c2cc(-c3ccccc3)nc(-c3ccc(-c4cc5c(-c6ccccc6)nc6ccccc6c5c5c4sc4ccccc45)cc3)n2)cc1 ; c1ccc(-c2cc(-c3ccc(-c4cc5c(-c6ccccc6)nc6ccccc6c5c5c4sc4ccccc45)cc3)nc(-c3ccccc3)n2)cc1 ; c1ccc(-c2ccc(-c3cc(-c4ccccc4)nc(-c4ccc(-c5cc6c(-c7ccccc7)nc7ccccc7c6c6c5sc5ccccc56)cc4)n3)cc2)cc1]

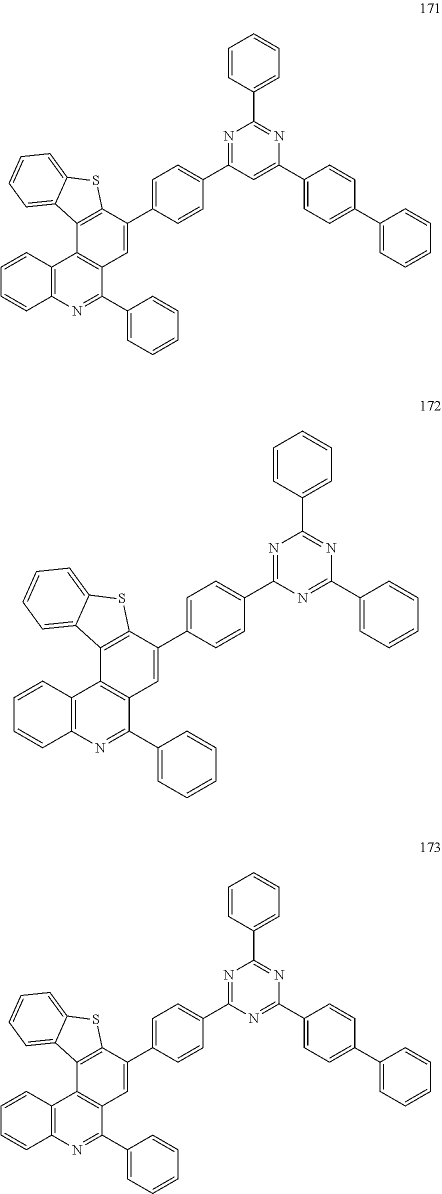 [SMILES: c1ccc(-c2ccc(-c3cc(-c4ccc(-c5cc6c(-c7ccccc7)nc7ccccc7c6c6c5sc5ccccc56)cc4)nc(-c4ccccc4)n3)cc2)cc1 ; c1ccc(-c2nc(-c3ccccc3)nc(-c3ccc(-c4cc5c(-c6ccccc6)nc6ccccc6c5c5c4sc4ccccc45)cc3)n2)cc1 ; c1ccc(-c2ccc(-c3nc(-c4ccccc4)nc(-c4ccc(-c5cc6c(-c7ccccc7)nc7ccccc7c6c6c5sc5ccccc56)cc4)n3)cc2)cc1]

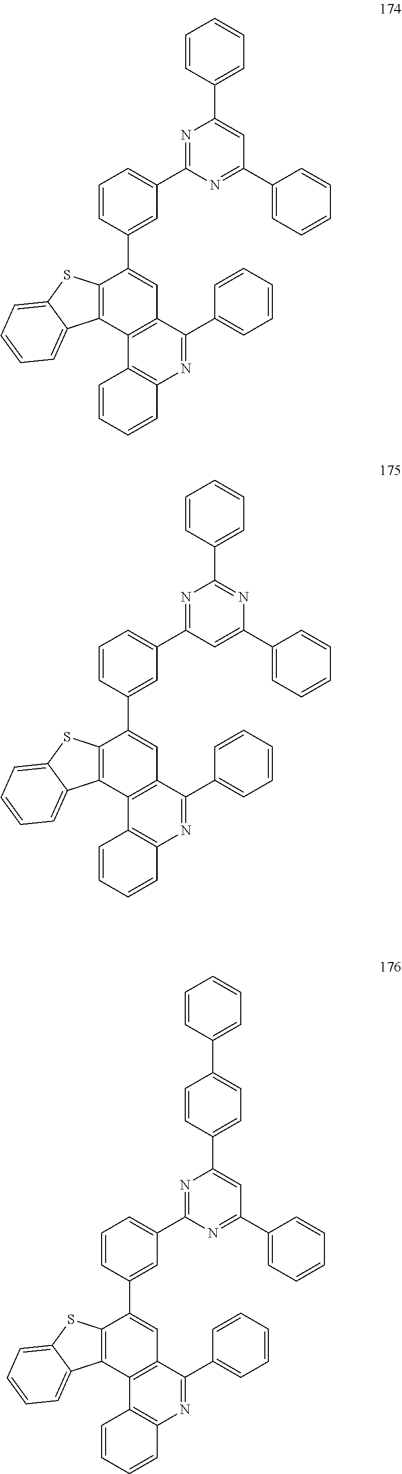 [SMILES: c1ccc(-c2cc(-c3ccccc3)nc(-c3cccc(-c4cc5c(-c6ccccc6)nc6ccccc6c5c5c4sc4ccccc45)c3)n2)cc1 ; c1ccc(-c2cc(-c3cccc(-c4cc5c(-c6ccccc6)nc6ccccc6c5c5c4sc4ccccc45)c3)nc(-c3ccccc3)n2)cc1 ; c1ccc(-c2ccc(-c3cc(-c4ccccc4)nc(-c4cccc(-c5cc6c(-c7ccccc7)nc7ccccc7c6c6c5sc5ccccc56)c4)n3)cc2)cc1]

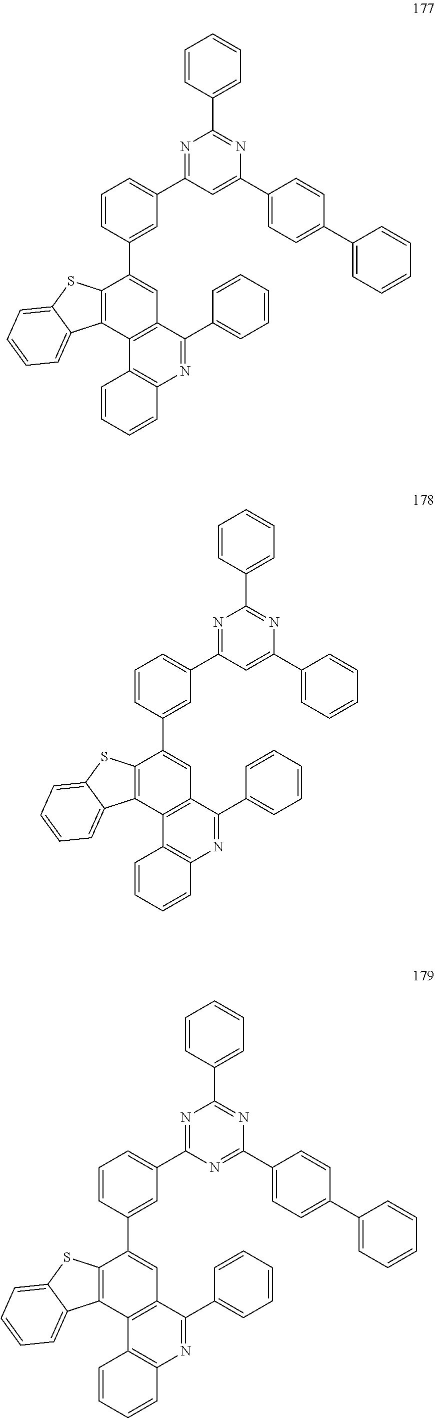 [SMILES: c1ccc(-c2ccc(-c3cc(-c4cccc(-c5cc6c(-c7ccccc7)nc7ccccc7c6c6c5sc5ccccc56)c4)nc(-c4ccccc4)n3)cc2)cc1 ; c1ccc(-c2cc(-c3cccc(-c4cc5c(-c6ccccc6)nc6ccccc6c5c5c4sc4ccccc45)c3)nc(-c3ccccc3)n2)cc1 ; c1ccc(-c2ccc(-c3nc(-c4ccccc4)nc(-c4cccc(-c5cc6c(-c7ccccc7)nc7ccccc7c6c6c5sc5ccccc56)c4)n3)cc2)cc1]

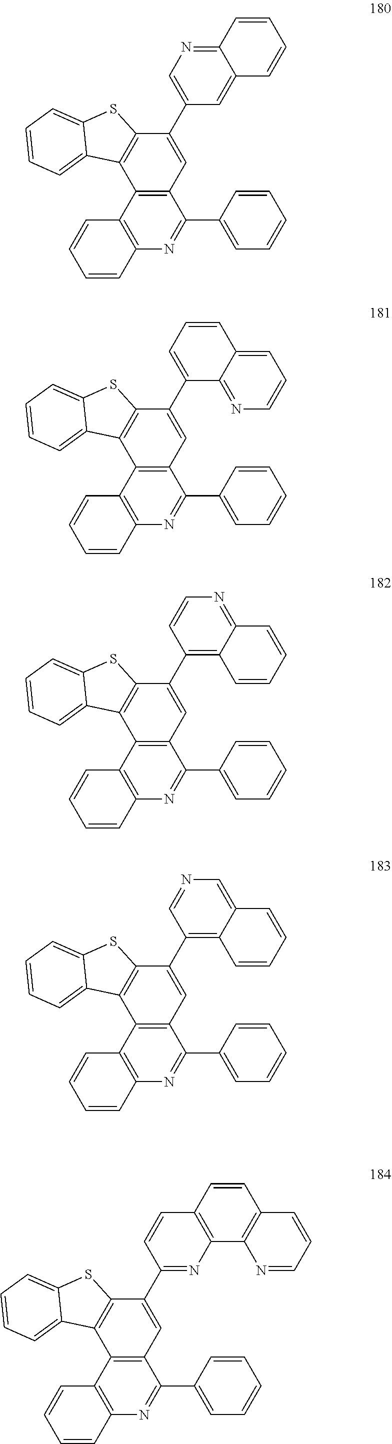 [SMILES: c1ccc(-c2nc3ccccc3c3c2cc(-c2cnc4ccccc4c2)c2sc4ccccc4c23)cc1 ; c1ccc(-c2nc3ccccc3c3c2cc(-c2cccc4cccnc24)c2sc4ccccc4c23)cc1 ; c1ccc(-c2nc3ccccc3c3c2cc(-c2ccnc4ccccc24)c2sc4ccccc4c23)cc1 ; c1ccc(-c2nc3ccccc3c3c2cc(-c2cncc4ccccc24)c2sc4ccccc4c23)cc1 ; c1ccc(-c2nc3ccccc3c3c2cc(-c2ccc4ccc5cccnc5c4n2)c2sc4ccccc4c23)cc1]

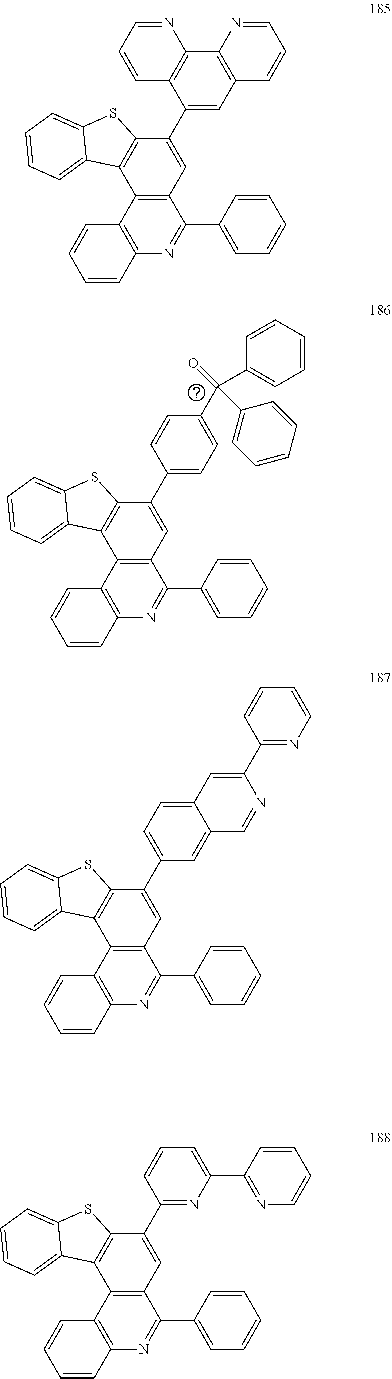

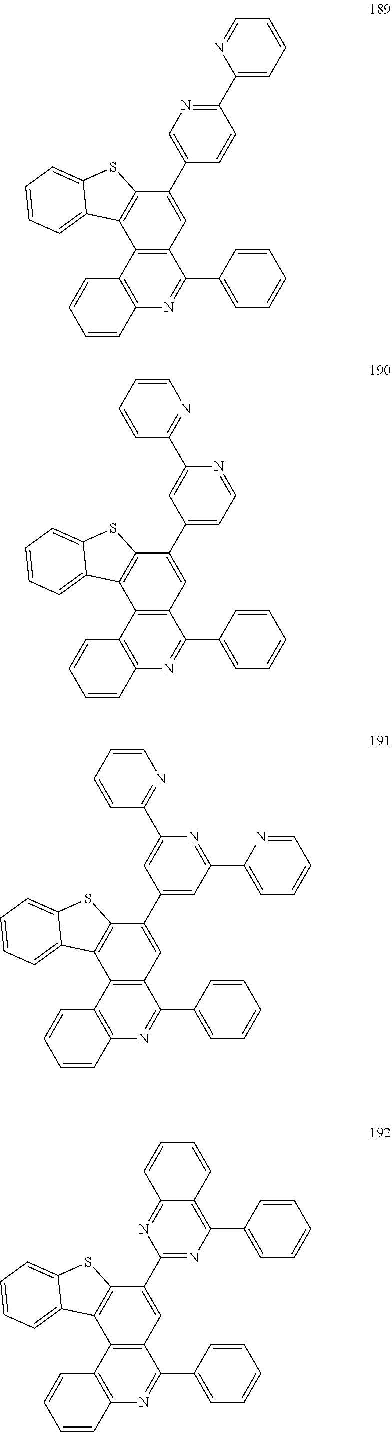 [SMILES: c1ccc(-c2nc3ccccc3c3c2cc(-c2ccc(-c4ccccn4)nc2)c2sc4ccccc4c23)cc1 ; c1ccc(-c2nc3ccccc3c3c2cc(-c2ccnc(-c4ccccn4)c2)c2sc4ccccc4c23)cc1 ; c1ccc(-c2nc3ccccc3c3c2cc(-c2cc(-c4ccccn4)nc(-c4ccccn4)c2)c2sc4ccccc4c23)cc1 ; c1ccc(-c2nc(-c3cc4c(-c5ccccc5)nc5ccccc5c4c4c3sc3ccccc34)nc3ccccc23)cc1]

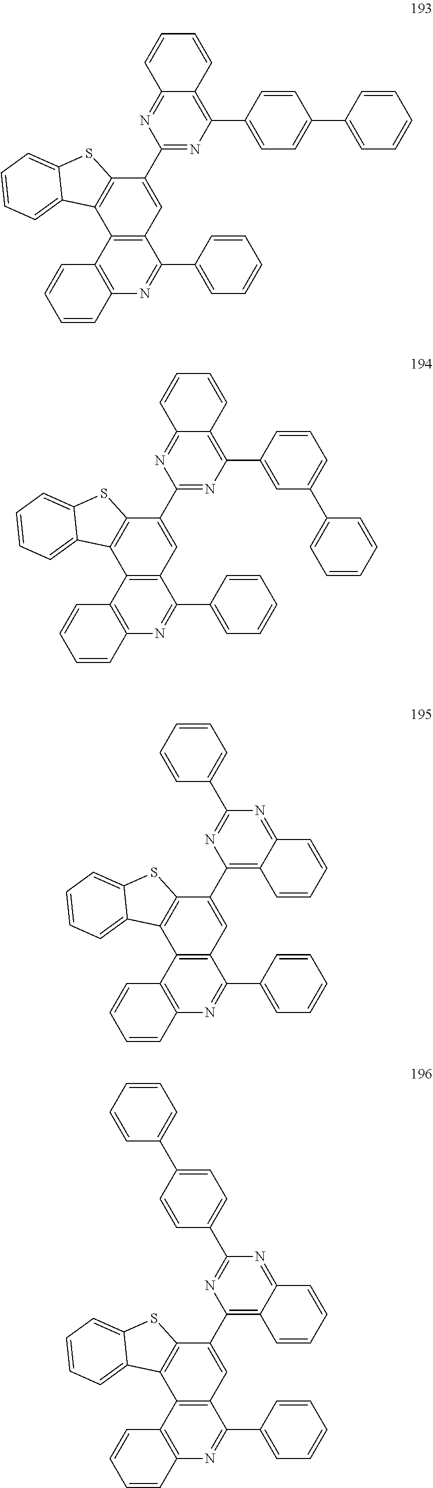

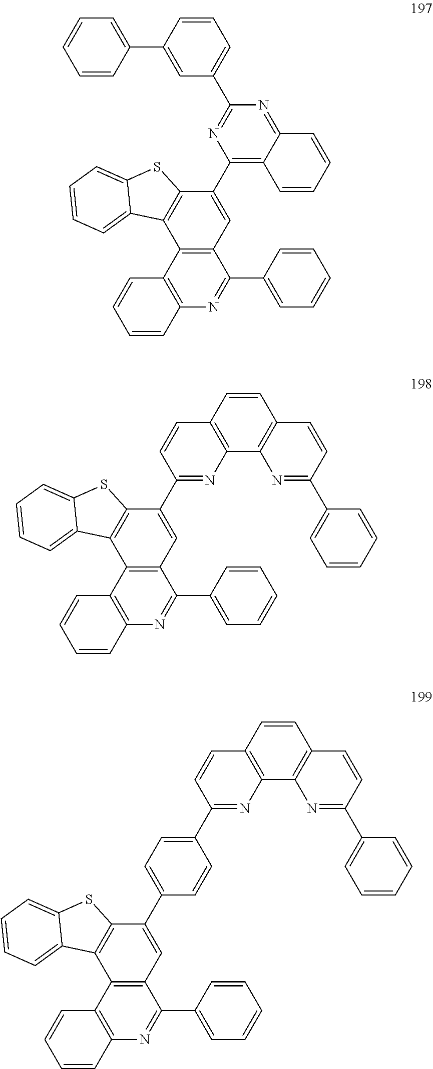 [SMILES: c1ccc(-c2cccc(-c3nc(-c4cc5c(-c6ccccc6)nc6ccccc6c5c5c4sc4ccccc45)c4ccccc4n3)c2)cc1 ; c1ccc(-c2ccc3ccc4ccc(-c5cc6c(-c7ccccc7)nc7ccccc7c6c6c5sc5ccccc56)nc4c3n2)cc1 ; c1ccc(-c2ccc3ccc4ccc(-c5ccc(-c6cc7c(-c8ccccc8)nc8ccccc8c7c7c6sc6ccccc67)cc5)nc4c3n2)cc1]

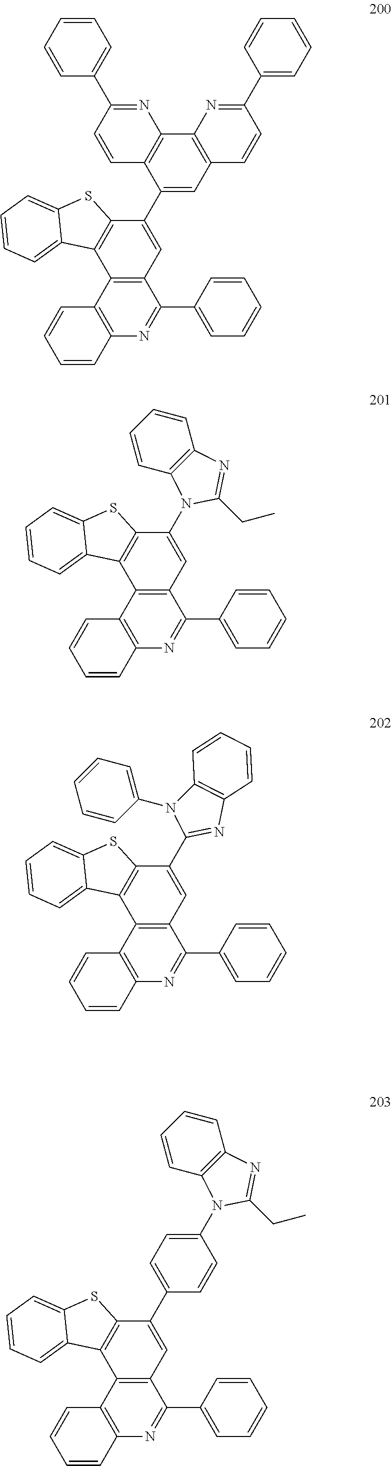 [SMILES: c1ccc(-c2ccc3cc(-c4cc5c(-c6ccccc6)nc6ccccc6c5c5c4sc4ccccc45)c4ccc(-c5ccccc5)nc4c3n2)cc1 ; CCc1nc2ccccc2n1-c1cc2c(-c3ccccc3)nc3ccccc3c2c2c1sc1ccccc12 ; c1ccc(-c2nc3ccccc3c3c2cc(-c2nc4ccccc4n2-c2ccccc2)c2sc4ccccc4c23)cc1 ; CCc1nc2ccccc2n1-c1ccc(-c2cc3c(-c4ccccc4)nc4ccccc4c3c3c2sc2ccccc23)cc1]

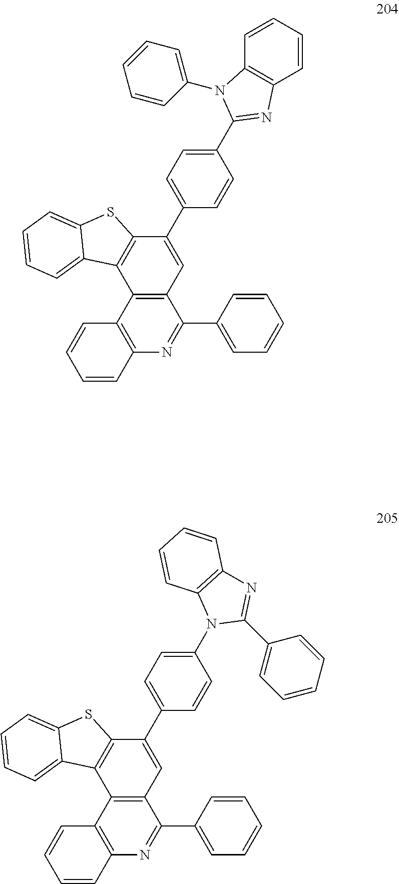 [SMILES: c1ccc(-c2nc3ccccc3c3c2cc(-c2ccc(-c4nc5ccccc5n4-c4ccccc4)cc2)c2sc4ccccc4c23)cc1 ; c1ccc(-c2nc3ccccc3c3c2cc(-c2ccc(-n4c(-c5ccccc5)nc5ccccc54)cc2)c2sc4ccccc4c23)cc1]

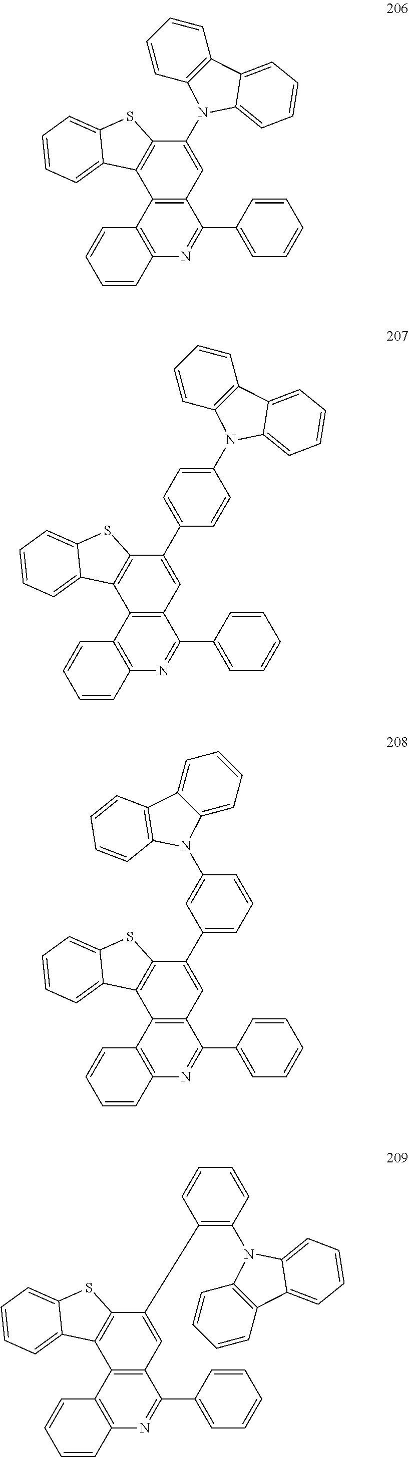 [SMILES: c1ccc(-c2nc3ccccc3c3c2cc(-n2c4ccccc4c4ccccc42)c2sc4ccccc4c23)cc1 ; c1ccc(-c2nc3ccccc3c3c2cc(-c2ccc(-n4c5ccccc5c5ccccc54)cc2)c2sc4ccccc4c23)cc1 ; c1ccc(-c2nc3ccccc3c3c2cc(-c2cccc(-n4c5ccccc5c5ccccc54)c2)c2sc4ccccc4c23)cc1 ; c1ccc(-c2nc3ccccc3c3c2cc(-c2ccccc2-n2c4ccccc4c4ccccc42)c2sc4ccccc4c23)cc1]

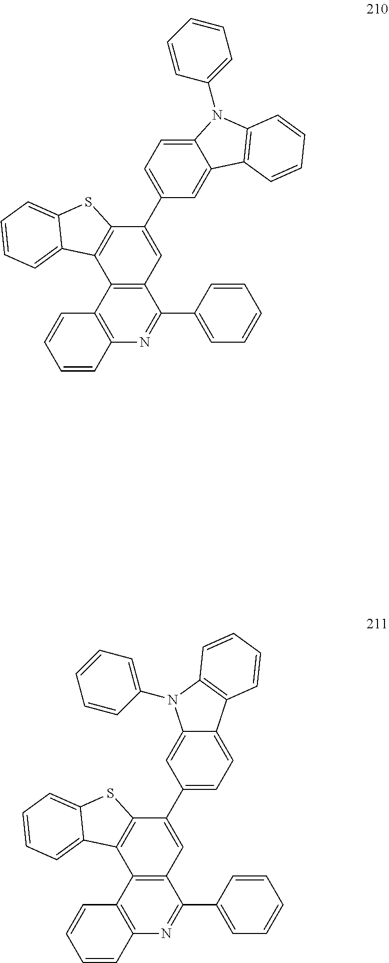 [SMILES: c1ccc(-c2nc3ccccc3c3c2cc(-c2ccc4c(c2)c2ccccc2n4-c2ccccc2)c2sc4ccccc4c23)cc1 ; c1ccc(-c2nc3ccccc3c3c2cc(-c2ccc4c5ccccc5n(-c5ccccc5)c4c2)c2sc4ccccc4c23)cc1]

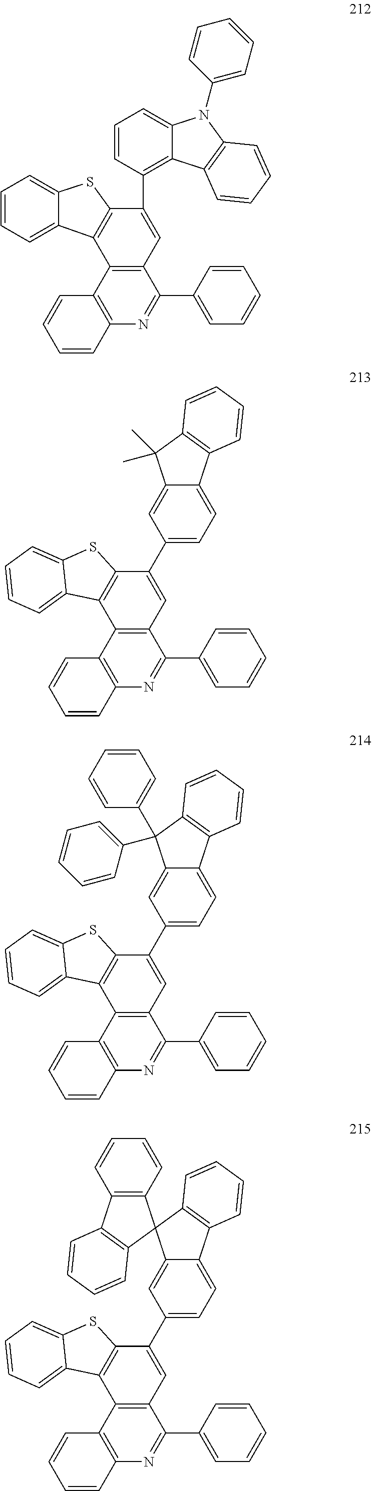

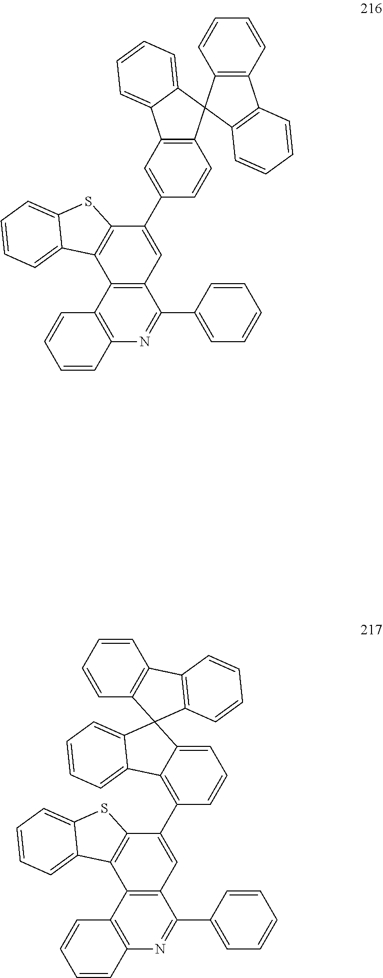 [SMILES: c1ccc(-c2nc3ccccc3c3c2cc(-c2ccc4c(c2)-c2ccccc2C42c4ccccc4-c4ccccc42)c2sc4ccccc4c23)cc1 ; c1ccc(-c2nc3ccccc3c3c2cc(-c2cccc4c2-c2ccccc2C42c4ccccc4-c4ccccc42)c2sc4ccccc4c23)cc1]

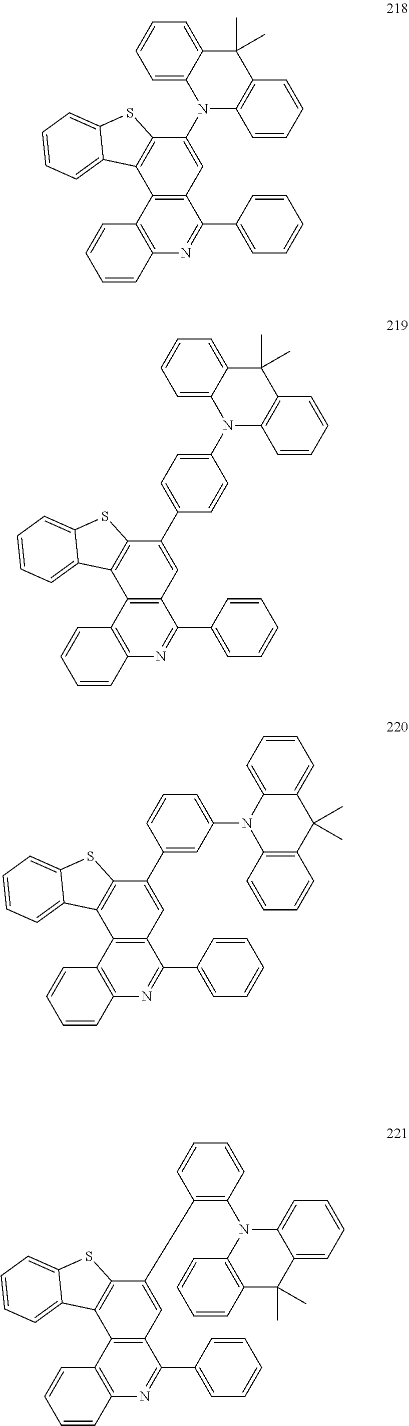 [SMILES: CC1(C)c2ccccc2N(c2cc3c(-c4ccccc4)nc4ccccc4c3c3c2sc2ccccc23)c2ccccc21 ; CC1(C)c2ccccc2N(c2ccc(-c3cc4c(-c5ccccc5)nc5ccccc5c4c4c3sc3ccccc34)cc2)c2ccccc21 ; CC1(C)c2ccccc2N(c2cccc(-c3cc4c(-c5ccccc5)nc5ccccc5c4c4c3sc3ccccc34)c2)c2ccccc21 ; CC1(C)c2ccccc2N(c2ccccc2-c2cc3c(-c4ccccc4)nc4ccccc4c3c3c2sc2ccccc23)c2ccccc21]

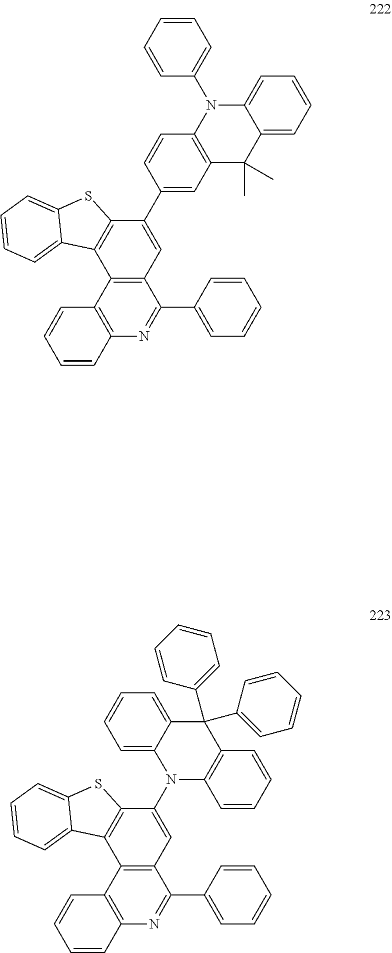 [SMILES: CC1(C)c2ccccc2N(c2ccccc2)c2ccc(-c3cc4c(-c5ccccc5)nc5ccccc5c4c4c3sc3ccccc34)cc21 ; c1ccc(-c2nc3ccccc3c3c2cc(N2c4ccccc4C(c4ccccc4)(c4ccccc4)c4ccccc42)c2sc4ccccc4c23)cc1]

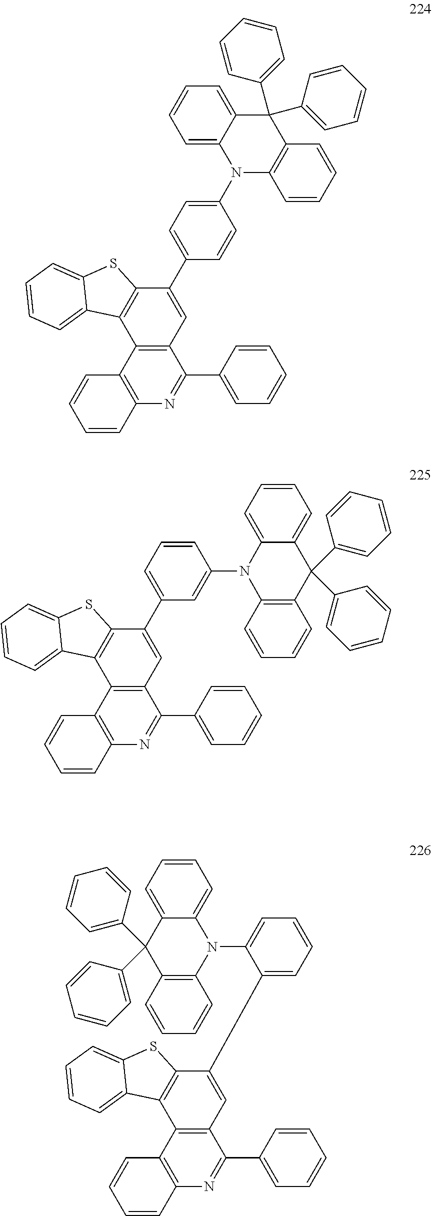 [SMILES: c1ccc(-c2nc3ccccc3c3c2cc(-c2ccc(N4c5ccccc5C(c5ccccc5)(c5ccccc5)c5ccccc54)cc2)c2sc4ccccc4c23)cc1 ; c1ccc(-c2nc3ccccc3c3c2cc(-c2cccc(N4c5ccccc5C(c5ccccc5)(c5ccccc5)c5ccccc54)c2)c2sc4ccccc4c23)cc1 ; c1ccc(-c2nc3ccccc3c3c2cc(-c2ccccc2N2c4ccccc4C(c4ccccc4)(c4ccccc4)c4ccccc42)c2sc4ccccc4c23)cc1]

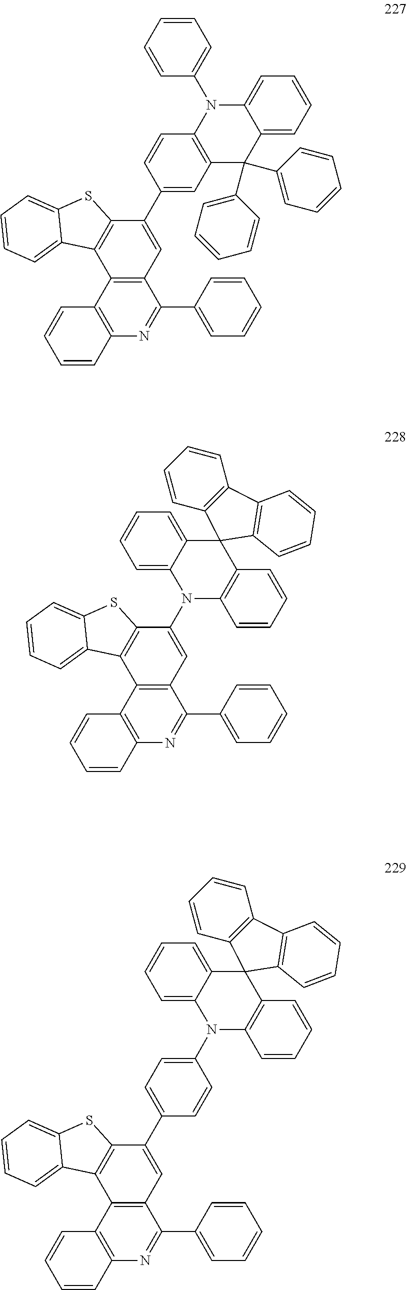 [SMILES: c1ccc(-c2nc3ccccc3c3c2cc(-c2ccc4c(c2)C(c2ccccc2)(c2ccccc2)c2ccccc2N4c2ccccc2)c2sc4ccccc4c23)cc1 ; c1ccc(-c2nc3ccccc3c3c2cc(N2c4ccccc4C4(c5ccccc5-c5ccccc54)c4ccccc42)c2sc4ccccc4c23)cc1 ; c1ccc(-c2nc3ccccc3c3c2cc(-c2ccc(N4c5ccccc5C5(c6ccccc6-c6ccccc65)c5ccccc54)cc2)c2sc4ccccc4c23)cc1]

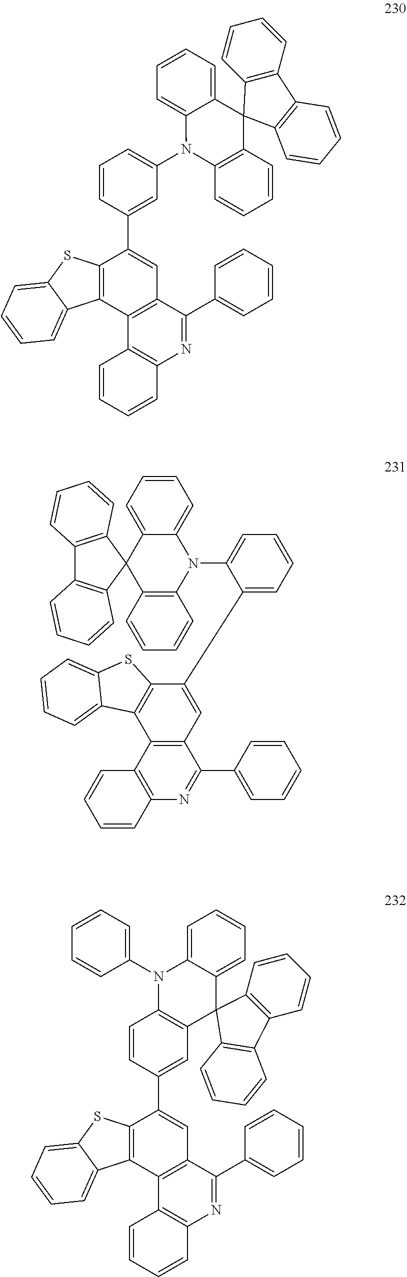 [SMILES: c1ccc(-c2nc3ccccc3c3c2cc(-c2cccc(N4c5ccccc5C5(c6ccccc6-c6ccccc65)c5ccccc54)c2)c2sc4ccccc4c23)cc1 ; c1ccc(-c2nc3ccccc3c3c2cc(-c2ccccc2N2c4ccccc4C4(c5ccccc5-c5ccccc54)c4ccccc42)c2sc4ccccc4c23)cc1 ; c1ccc(-c2nc3ccccc3c3c2cc(-c2ccc4c(c2)C2(c5ccccc5-c5ccccc52)c2ccccc2N4c2ccccc2)c2sc4ccccc4c23)cc1]

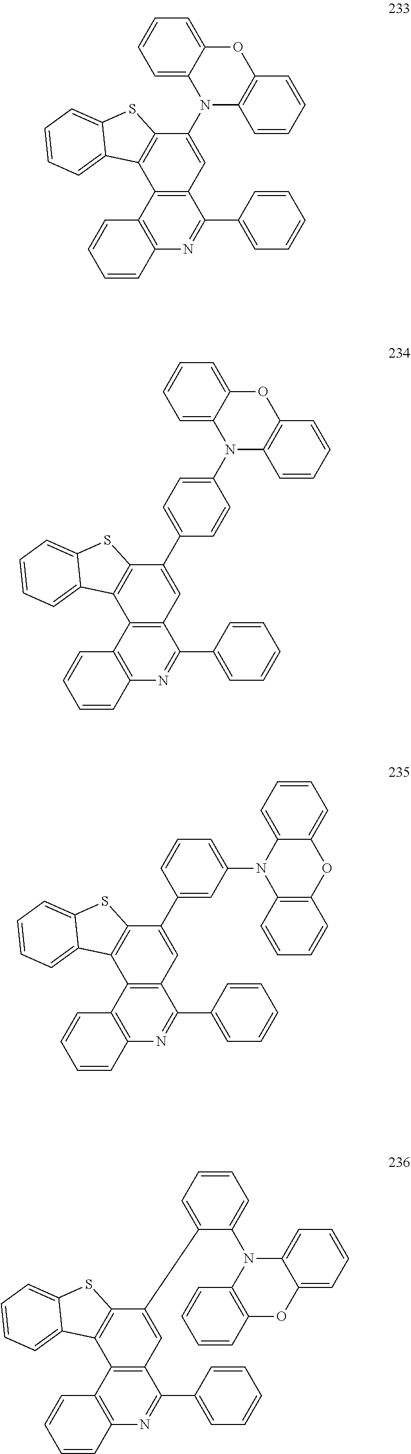 [SMILES: c1ccc(-c2nc3ccccc3c3c2cc(N2c4ccccc4Oc4ccccc42)c2sc4ccccc4c23)cc1 ; c1ccc(-c2nc3ccccc3c3c2cc(-c2ccc(N4c5ccccc5Oc5ccccc54)cc2)c2sc4ccccc4c23)cc1 ; c1ccc(-c2nc3ccccc3c3c2cc(-c2cccc(N4c5ccccc5Oc5ccccc54)c2)c2sc4ccccc4c23)cc1 ; c1ccc(-c2nc3ccccc3c3c2cc(-c2ccccc2N2c4ccccc4Oc4ccccc42)c2sc4ccccc4c23)cc1]

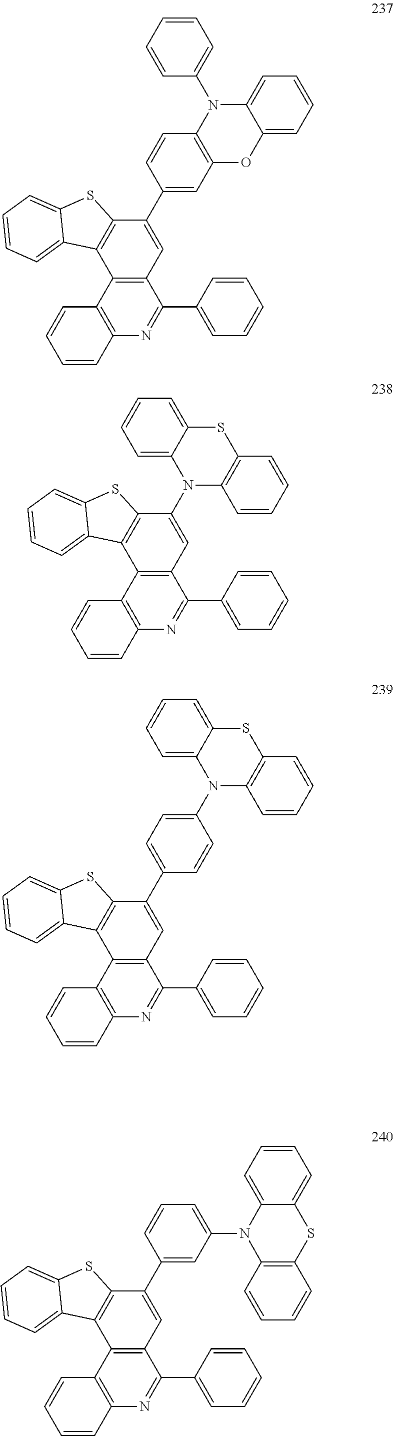 [SMILES: c1ccc(-c2nc3ccccc3c3c2cc(-c2ccc4c(c2)Oc2ccccc2N4c2ccccc2)c2sc4ccccc4c23)cc1 ; c1ccc(-c2nc3ccccc3c3c2cc(N2c4ccccc4Sc4ccccc42)c2sc4ccccc4c23)cc1 ; c1ccc(-c2nc3ccccc3c3c2cc(-c2ccc(N4c5ccccc5Sc5ccccc54)cc2)c2sc4ccccc4c23)cc1 ; c1ccc(-c2nc3ccccc3c3c2cc(-c2cccc(N4c5ccccc5Sc5ccccc54)c2)c2sc4ccccc4c23)cc1]

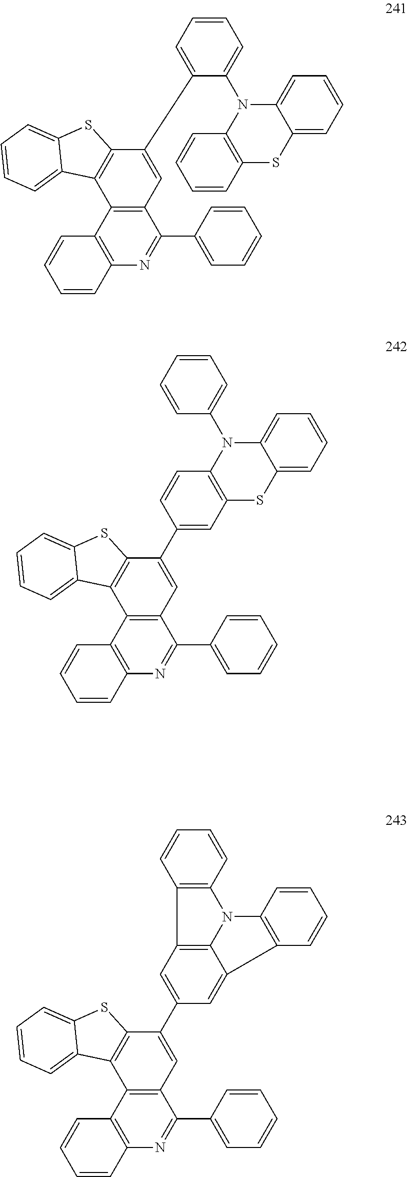 [SMILES: c1ccc(-c2nc3ccccc3c3c2cc(-c2ccccc2N2c4ccccc4Sc4ccccc42)c2sc4ccccc4c23)cc1 ; c1ccc(-c2nc3ccccc3c3c2cc(-c2ccc4c(c2)Sc2ccccc2N4c2ccccc2)c2sc4ccccc4c23)cc1 ; c1ccc(-c2nc3ccccc3c3c2cc(-c2cc4c5ccccc5n5c6ccccc6c(c2)c45)c2sc4ccccc4c23)cc1]

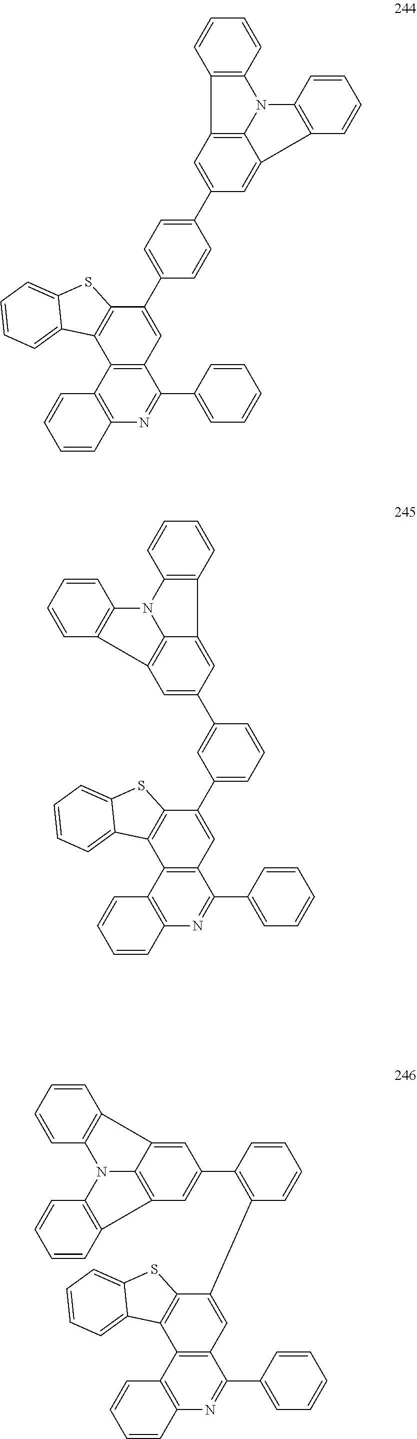 [SMILES: c1ccc(-c2nc3ccccc3c3c2cc(-c2ccc(-c4cc5c6ccccc6n6c7ccccc7c(c4)c56)cc2)c2sc4ccccc4c23)cc1 ; c1ccc(-c2nc3ccccc3c3c2cc(-c2cccc(-c4cc5c6ccccc6n6c7ccccc7c(c4)c56)c2)c2sc4ccccc4c23)cc1 ; c1ccc(-c2nc3ccccc3c3c2cc(-c2ccccc2-c2cc4c5ccccc5n5c6ccccc6c(c2)c45)c2sc4ccccc4c23)cc1]

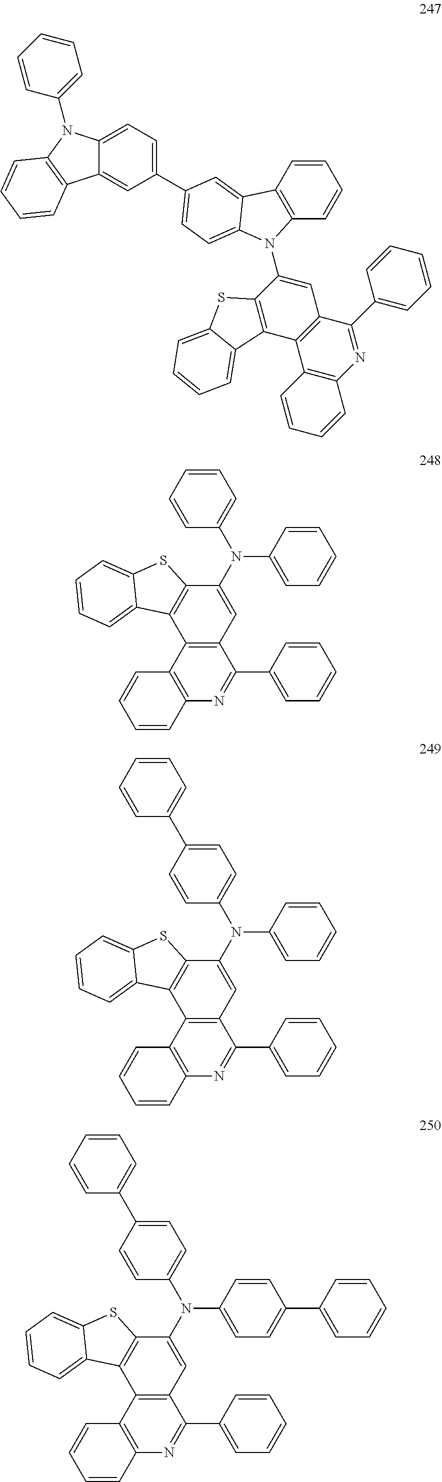

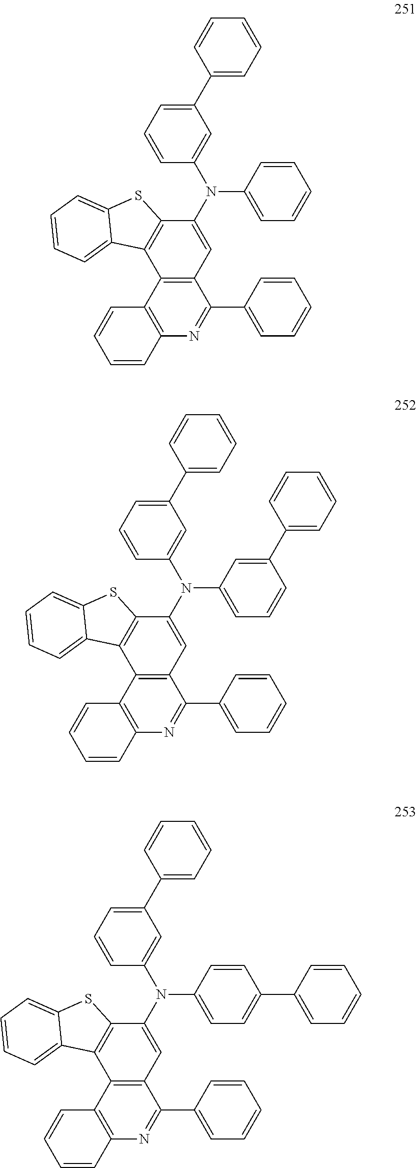 [SMILES: c1ccc(-c2cccc(N(c3ccccc3)c3cc4c(-c5ccccc5)nc5ccccc5c4c4c3sc3ccccc34)c2)cc1 ; c1ccc(-c2cccc(N(c3cccc(-c4ccccc4)c3)c3cc4c(-c5ccccc5)nc5ccccc5c4c4c3sc3ccccc34)c2)cc1 ; c1ccc(-c2ccc(N(c3cccc(-c4ccccc4)c3)c3cc4c(-c5ccccc5)nc5ccccc5c4c4c3sc3ccccc34)cc2)cc1]

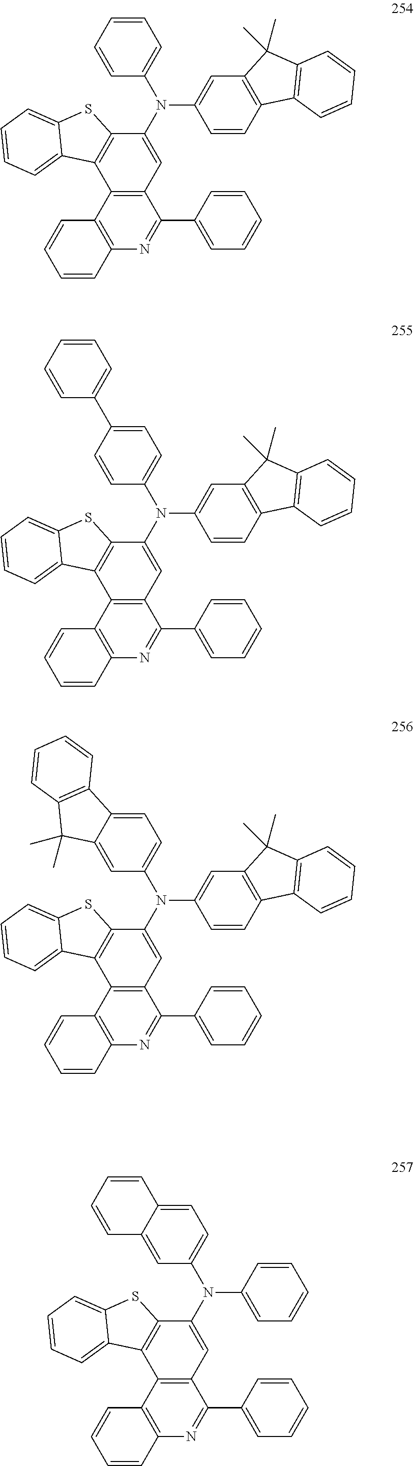 [SMILES: CC1(C)c2ccccc2-c2ccc(N(c3ccccc3)c3cc4c(-c5ccccc5)nc5ccccc5c4c4c3sc3ccccc34)cc21 ; CC1(C)c2ccccc2-c2ccc(N(c3ccc(-c4ccccc4)cc3)c3cc4c(-c5ccccc5)nc5ccccc5c4c4c3sc3ccccc34)cc21 ; CC1(C)c2ccccc2-c2ccc(N(c3ccc4c(c3)C(C)(C)c3ccccc3-4)c3cc4c(-c5ccccc5)nc5ccccc5c4c4c3sc3ccccc34)cc21 ; c1ccc(-c2nc3ccccc3c3c2cc(N(c2ccccc2)c2ccc4ccccc4c2)c2sc4ccccc4c23)cc1]

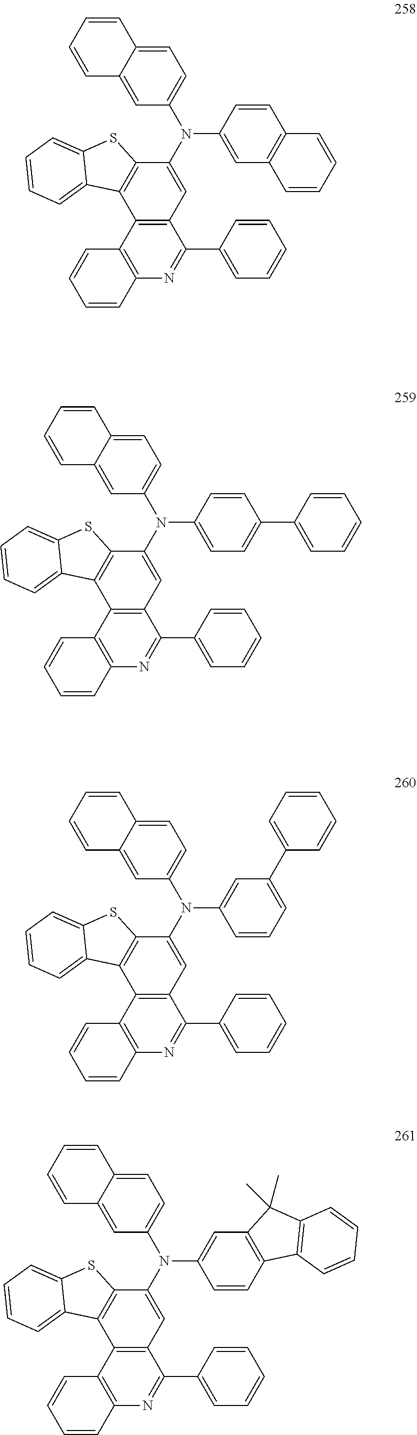 [SMILES: c1ccc(-c2nc3ccccc3c3c2cc(N(c2ccc4ccccc4c2)c2ccc4ccccc4c2)c2sc4ccccc4c23)cc1 ; c1ccc(-c2ccc(N(c3ccc4ccccc4c3)c3cc4c(-c5ccccc5)nc5ccccc5c4c4c3sc3ccccc34)cc2)cc1 ; c1ccc(-c2cccc(N(c3ccc4ccccc4c3)c3cc4c(-c5ccccc5)nc5ccccc5c4c4c3sc3ccccc34)c2)cc1 ; CC1(C)c2ccccc2-c2ccc(N(c3ccc4ccccc4c3)c3cc4c(-c5ccccc5)nc5ccccc5c4c4c3sc3ccccc34)cc21]

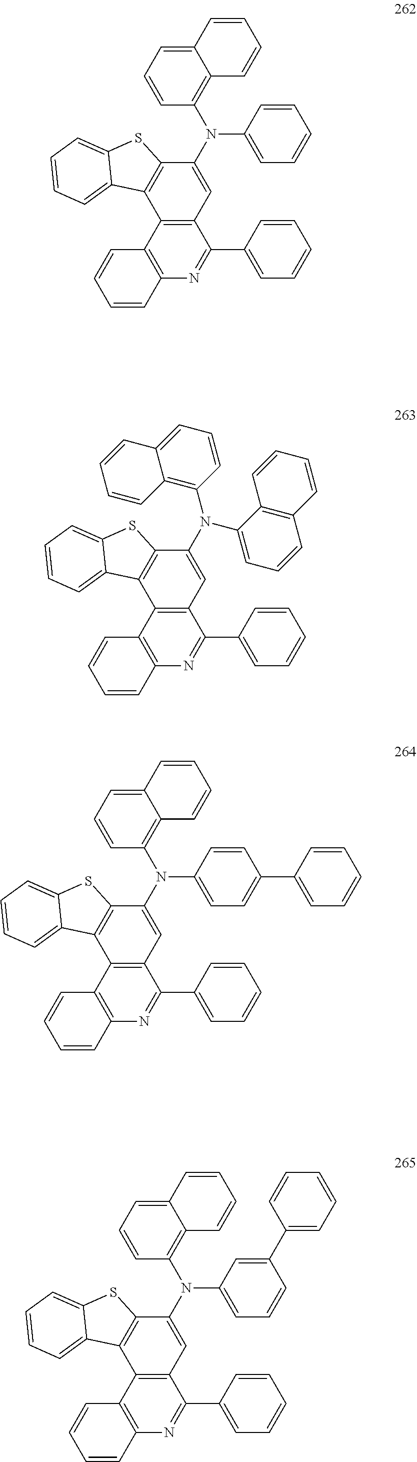 [SMILES: c1ccc(-c2nc3ccccc3c3c2cc(N(c2ccccc2)c2cccc4ccccc24)c2sc4ccccc4c23)cc1 ; c1ccc(-c2nc3ccccc3c3c2cc(N(c2cccc4ccccc24)c2cccc4ccccc24)c2sc4ccccc4c23)cc1 ; c1ccc(-c2ccc(N(c3cccc4ccccc34)c3cc4c(-c5ccccc5)nc5ccccc5c4c4c3sc3ccccc34)cc2)cc1 ; c1ccc(-c2cccc(N(c3cccc4ccccc34)c3cc4c(-c5ccccc5)nc5ccccc5c4c4c3sc3ccccc34)c2)cc1]

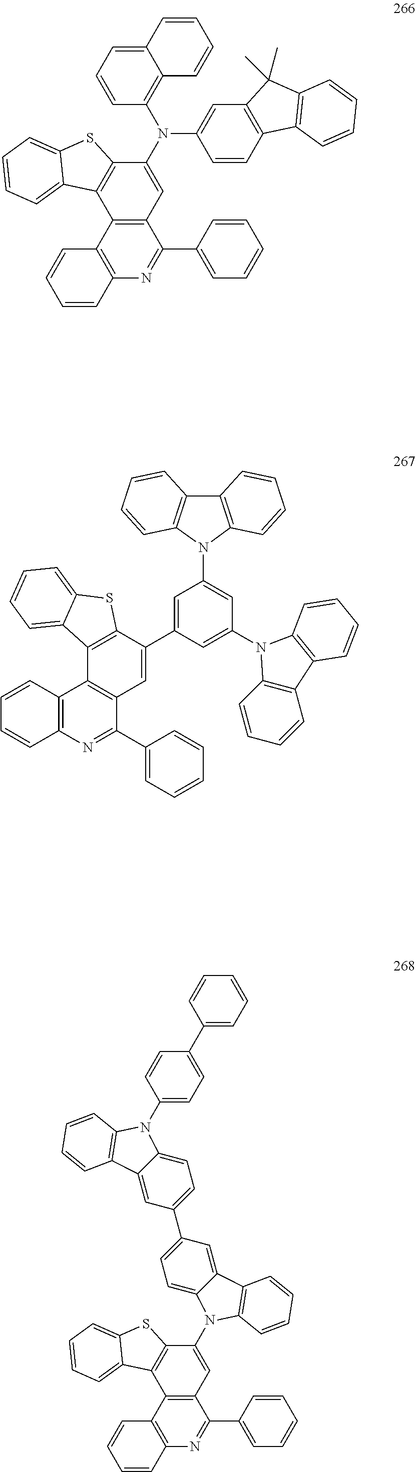 [SMILES: CC1(C)c2ccccc2-c2ccc(N(c3cccc4ccccc34)c3cc4c(-c5ccccc5)nc5ccccc5c4c4c3sc3ccccc34)cc21 ; c1ccc(-c2nc3ccccc3c3c2cc(-c2cc(-n4c5ccccc5c5ccccc54)cc(-n4c5ccccc5c5ccccc54)c2)c2sc4ccccc4c23)cc1 ; c1ccc(-c2ccc(-n3c4ccccc4c4cc(-c5ccc6c(c5)c5ccccc5n6-c5cc6c(-c7ccccc7)nc7ccccc7c6c6c5sc5ccccc56)ccc43)cc2)cc1]

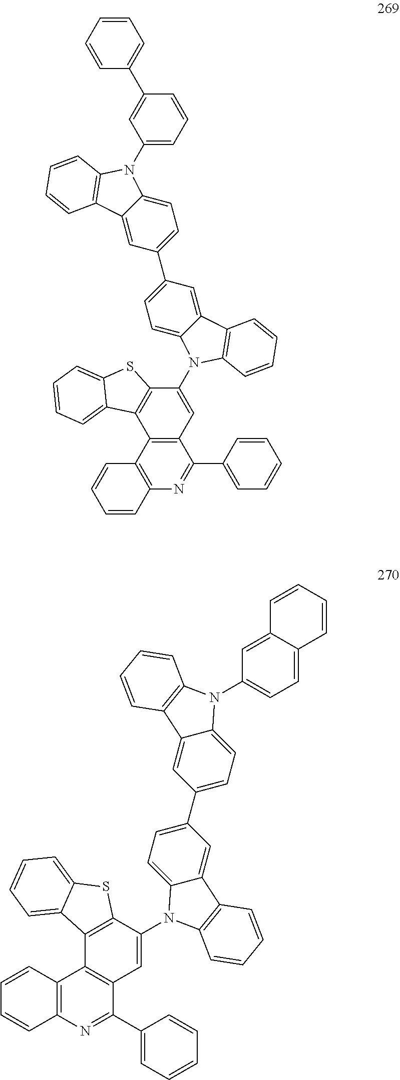 [SMILES: c1ccc(-c2cccc(-n3c4ccccc4c4cc(-c5ccc6c(c5)c5ccccc5n6-c5cc6c(-c7ccccc7)nc7ccccc7c6c6c5sc5ccccc56)ccc43)c2)cc1 ; c1ccc(-c2nc3ccccc3c3c2cc(-n2c4ccccc4c4cc(-c5ccc6c(c5)c5ccccc5n6-c5ccc6ccccc6c5)ccc42)c2sc4ccccc4c23)cc1]

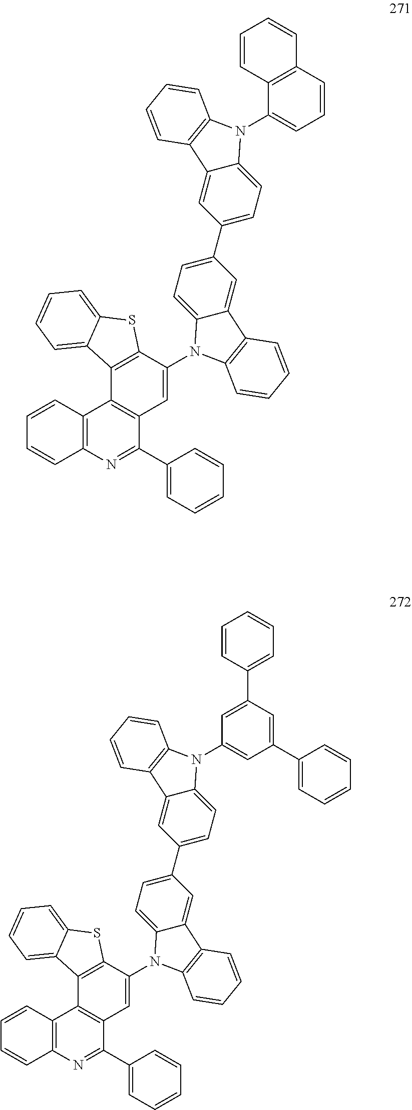 [SMILES: c1ccc(-c2nc3ccccc3c3c2cc(-n2c4ccccc4c4cc(-c5ccc6c(c5)c5ccccc5n6-c5cccc6ccccc56)ccc42)c2sc4ccccc4c23)cc1 ; c1ccc(-c2cc(-c3ccccc3)cc(-n3c4ccccc4c4cc(-c5ccc6c(c5)c5ccccc5n6-c5cc6c(-c7ccccc7)nc7ccccc7c6c6c5sc5ccccc56)ccc43)c2)cc1]

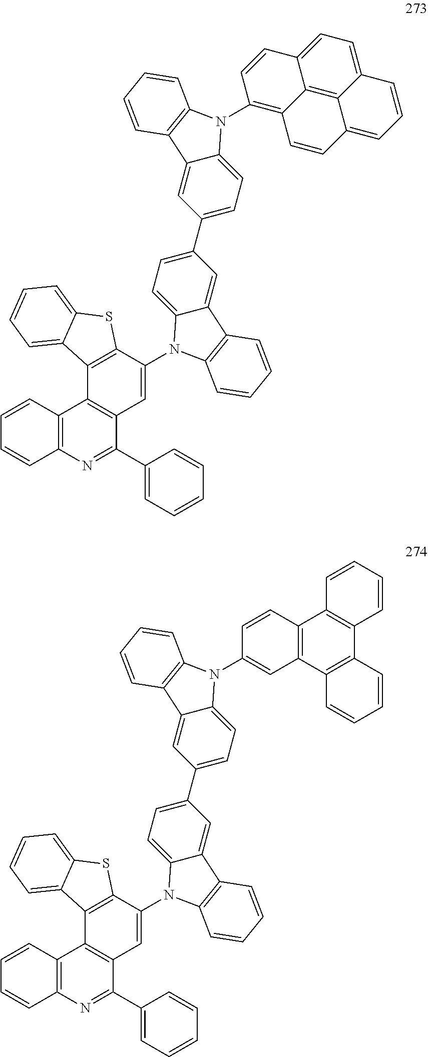 [SMILES: c1ccc(-c2nc3ccccc3c3c2cc(-n2c4ccccc4c4cc(-c5ccc6c(c5)c5ccccc5n6-c5ccc6ccc7cccc8ccc5c6c78)ccc42)c2sc4ccccc4c23)cc1 ; c1ccc(-c2nc3ccccc3c3c2cc(-n2c4ccccc4c4cc(-c5ccc6c(c5)c5ccccc5n6-c5ccc6c7ccccc7c7ccccc7c6c5)ccc42)c2sc4ccccc4c23)cc1]

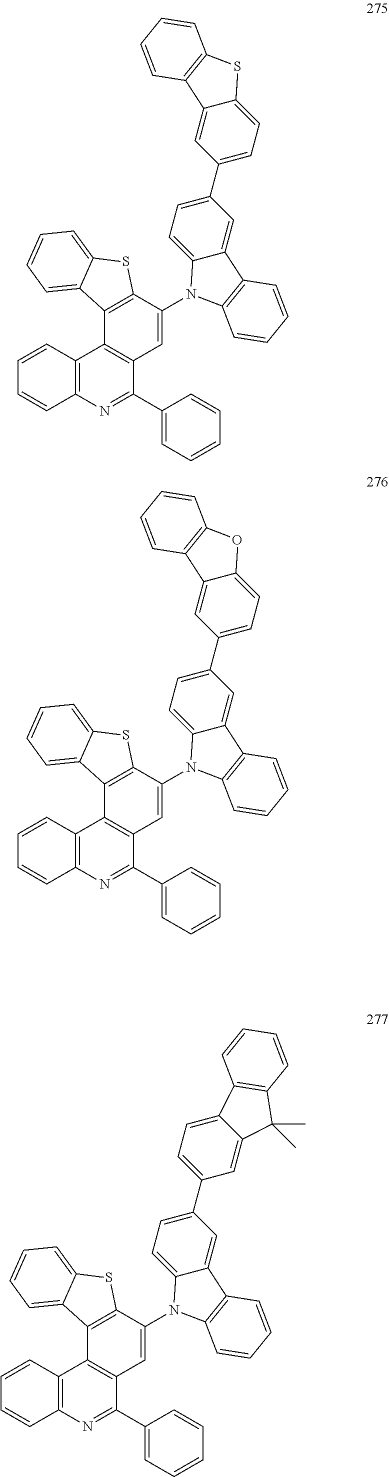

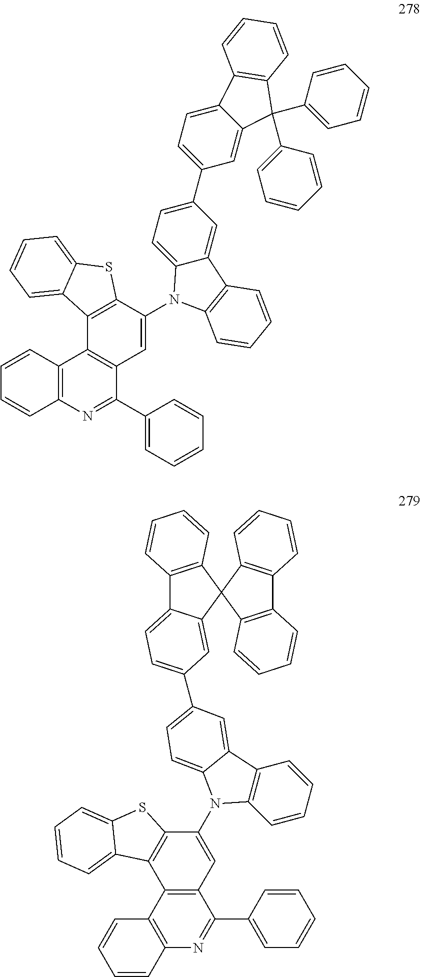

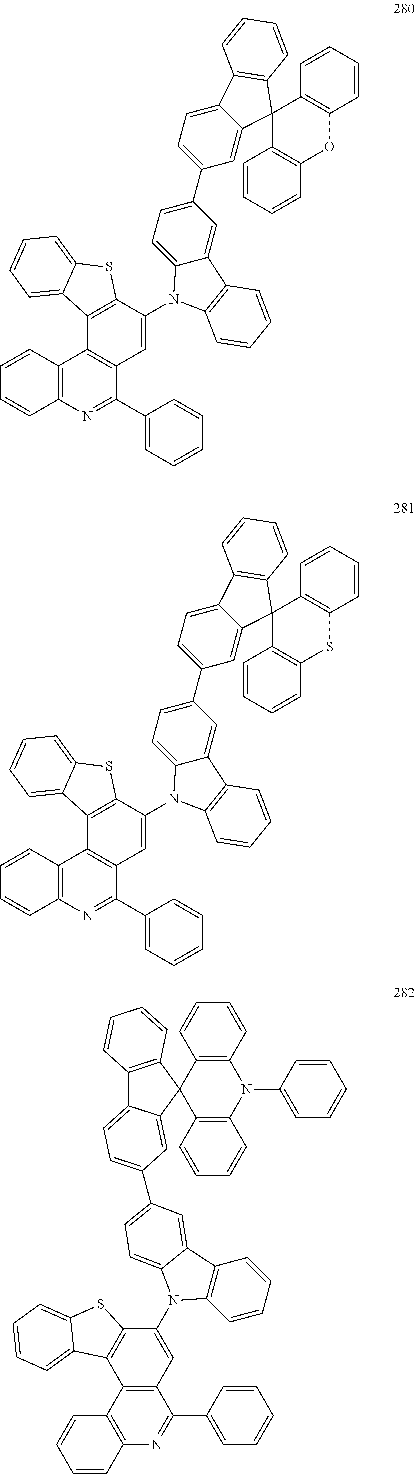

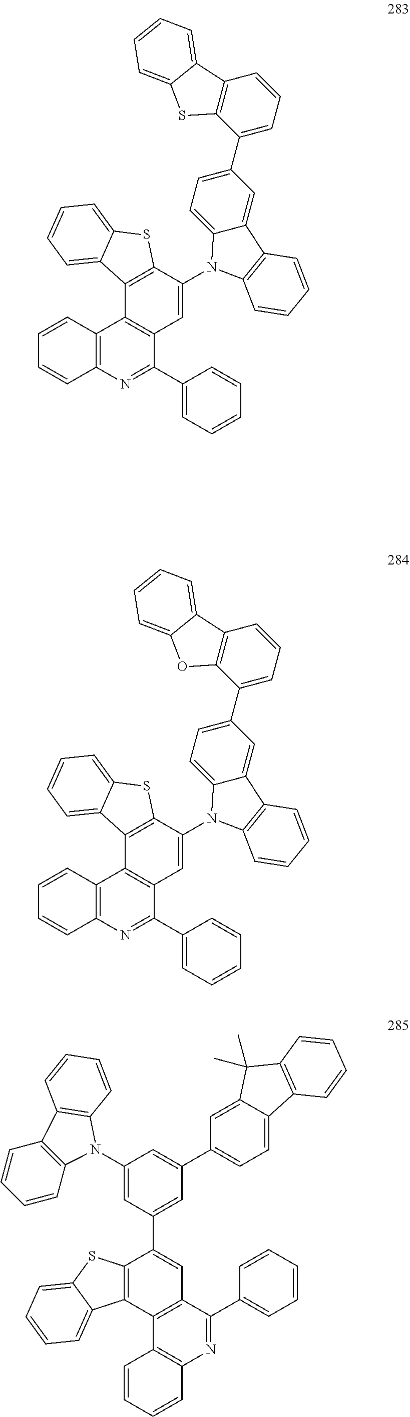 [SMILES: c1ccc(-c2nc3ccccc3c3c2cc(-n2c4ccccc4c4cc(-c5cccc6c5sc5ccccc56)ccc42)c2sc4ccccc4c23)cc1 ; c1ccc(-c2nc3ccccc3c3c2cc(-n2c4ccccc4c4cc(-c5cccc6c5oc5ccccc56)ccc42)c2sc4ccccc4c23)cc1 ; CC1(C)c2ccccc2-c2ccc(-c3cc(-c4cc5c(-c6ccccc6)nc6ccccc6c5c5c4sc4ccccc45)cc(-n4c5ccccc5c5ccccc54)c3)cc21]

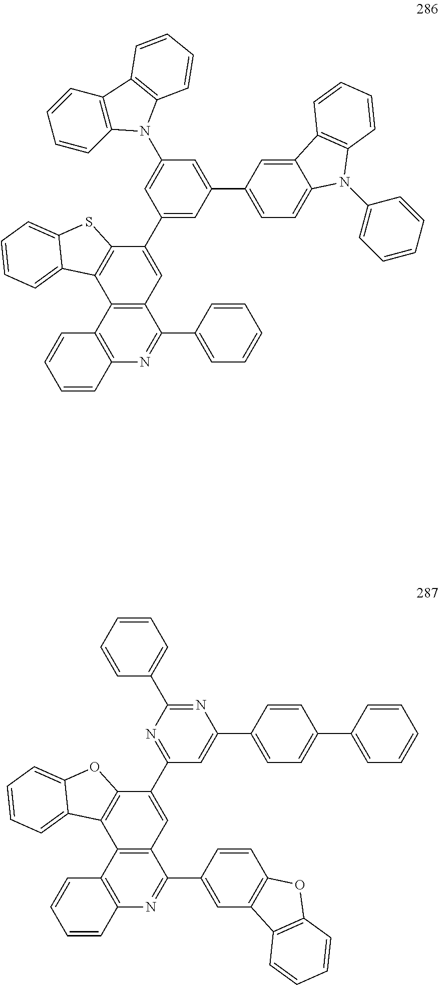 [SMILES: c1ccc(-c2nc3ccccc3c3c2cc(-c2cc(-c4ccc5c(c4)c4ccccc4n5-c4ccccc4)cc(-n4c5ccccc5c5ccccc54)c2)c2sc4ccccc4c23)cc1 ; c1ccc(-c2ccc(-c3cc(-c4cc5c(-c6ccc7oc8ccccc8c7c6)nc6ccccc6c5c5c4oc4ccccc45)nc(-c4ccccc4)n3)cc2)cc1]

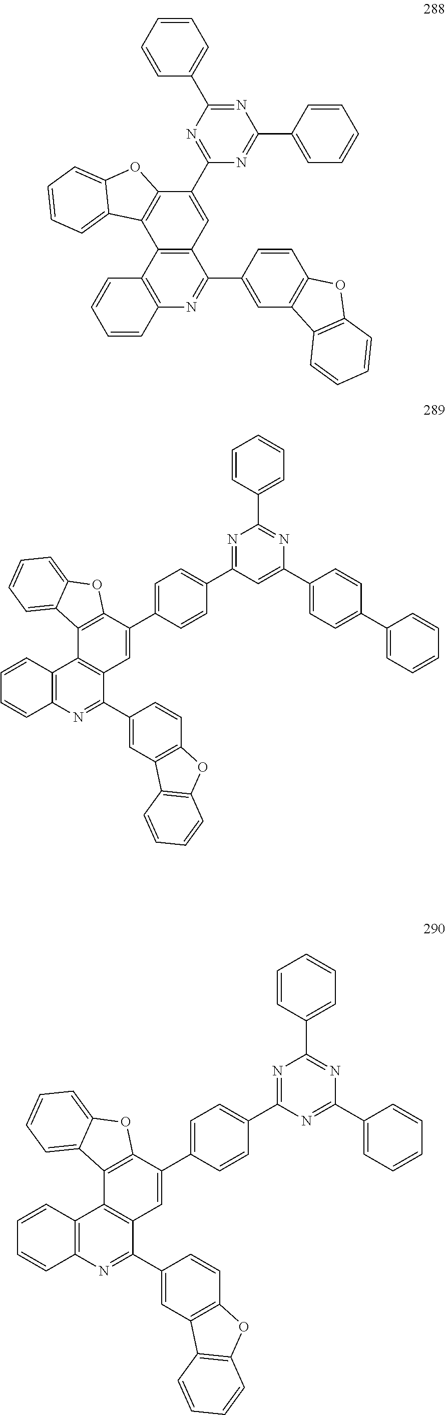 [SMILES: c1ccc(-c2nc(-c3ccccc3)nc(-c3cc4c(-c5ccc6oc7ccccc7c6c5)nc5ccccc5c4c4c3oc3ccccc34)n2)cc1 ; c1ccc(-c2ccc(-c3cc(-c4ccc(-c5cc6c(-c7ccc8oc9ccccc9c8c7)nc7ccccc7c6c6c5oc5ccccc56)cc4)nc(-c4ccccc4)n3)cc2)cc1 ; c1ccc(-c2nc(-c3ccccc3)nc(-c3ccc(-c4cc5c(-c6ccc7oc8ccccc8c7c6)nc6ccccc6c5c5c4oc4ccccc45)cc3)n2)cc1]

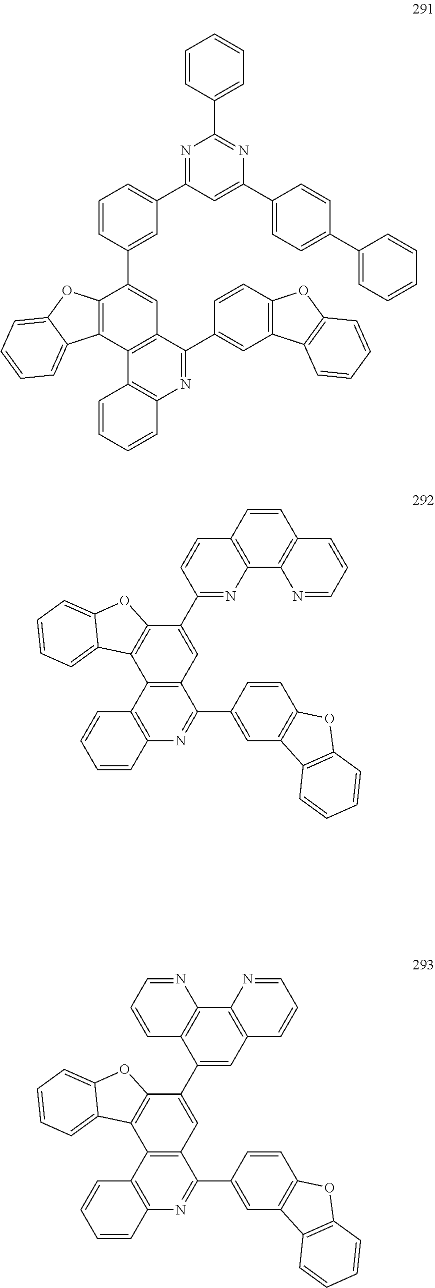 [SMILES: c1ccc(-c2ccc(-c3cc(-c4cccc(-c5cc6c(-c7ccc8oc9ccccc9c8c7)nc7ccccc7c6c6c5oc5ccccc56)c4)nc(-c4ccccc4)n3)cc2)cc1 ; c1cnc2c(c1)ccc1ccc(-c3cc4c(-c5ccc6oc7ccccc7c6c5)nc5ccccc5c4c4c3oc3ccccc34)nc12 ; c1cnc2c(c1)cc(-c1cc3c(-c4ccc5oc6ccccc6c5c4)nc4ccccc4c3c3c1oc1ccccc13)c1cccnc12]

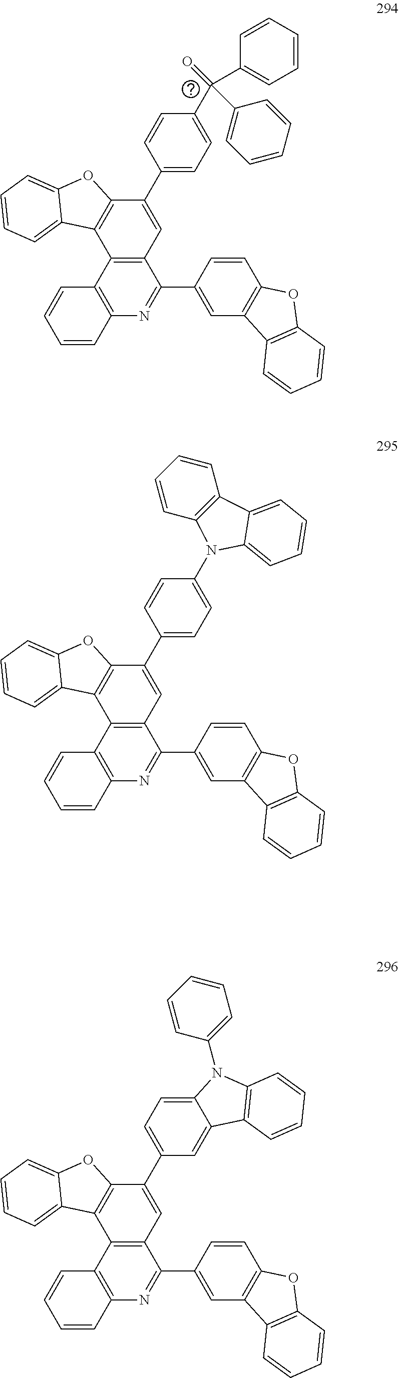

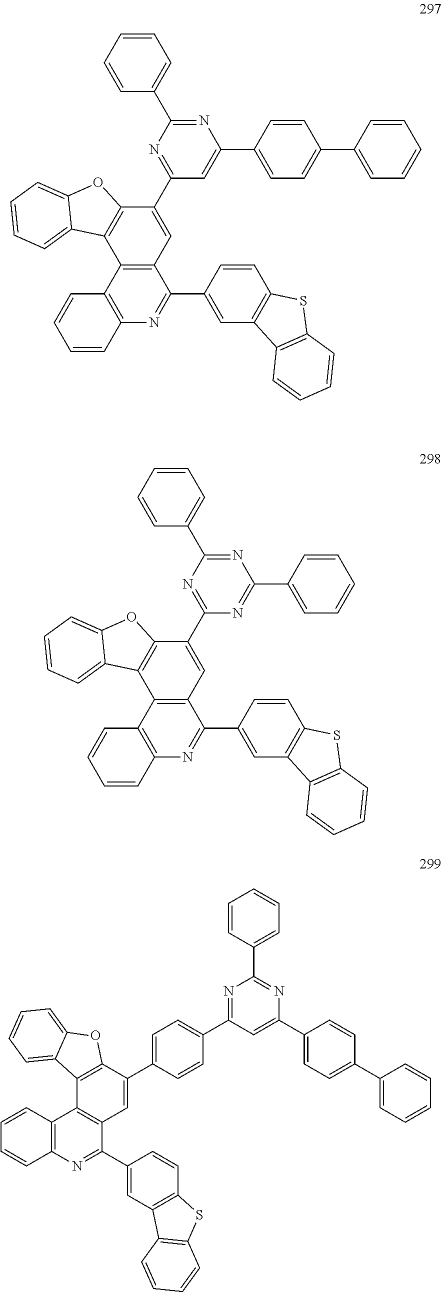 [SMILES: c1ccc(-c2ccc(-c3cc(-c4cc5c(-c6ccc7sc8ccccc8c7c6)nc6ccccc6c5c5c4oc4ccccc45)nc(-c4ccccc4)n3)cc2)cc1 ; c1ccc(-c2nc(-c3ccccc3)nc(-c3cc4c(-c5ccc6sc7ccccc7c6c5)nc5ccccc5c4c4c3oc3ccccc34)n2)cc1 ; c1ccc(-c2ccc(-c3cc(-c4ccc(-c5cc6c(-c7ccc8sc9ccccc9c8c7)nc7ccccc7c6c6c5oc5ccccc56)cc4)nc(-c4ccccc4)n3)cc2)cc1]

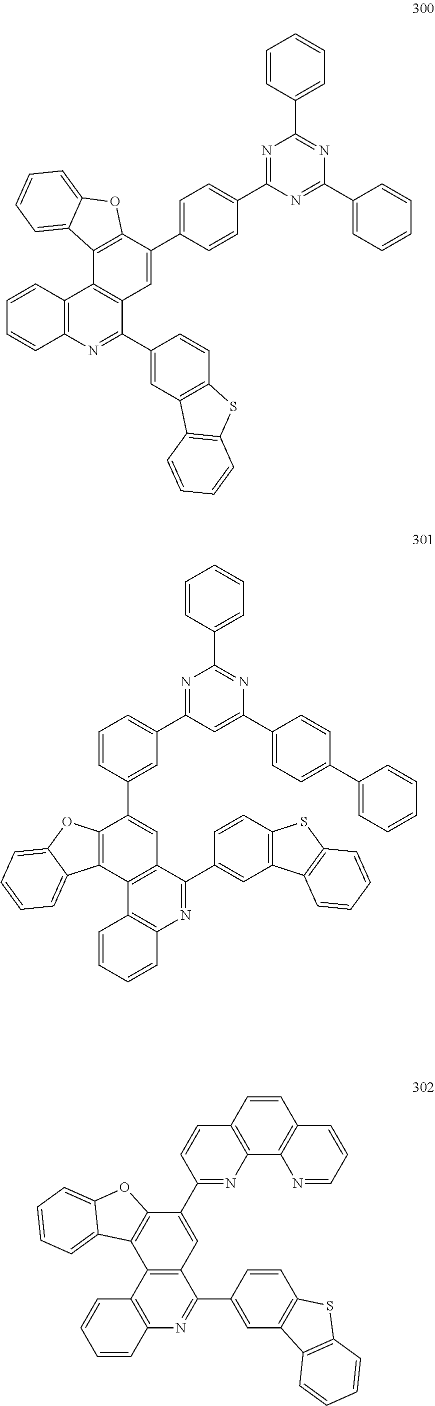 [SMILES: c1ccc(-c2nc(-c3ccccc3)nc(-c3ccc(-c4cc5c(-c6ccc7sc8ccccc8c7c6)nc6ccccc6c5c5c4oc4ccccc45)cc3)n2)cc1 ; c1ccc(-c2ccc(-c3cc(-c4cccc(-c5cc6c(-c7ccc8sc9ccccc9c8c7)nc7ccccc7c6c6c5oc5ccccc56)c4)nc(-c4ccccc4)n3)cc2)cc1 ; c1cnc2c(c1)ccc1ccc(-c3cc4c(-c5ccc6sc7ccccc7c6c5)nc5ccccc5c4c4c3oc3ccccc34)nc12]

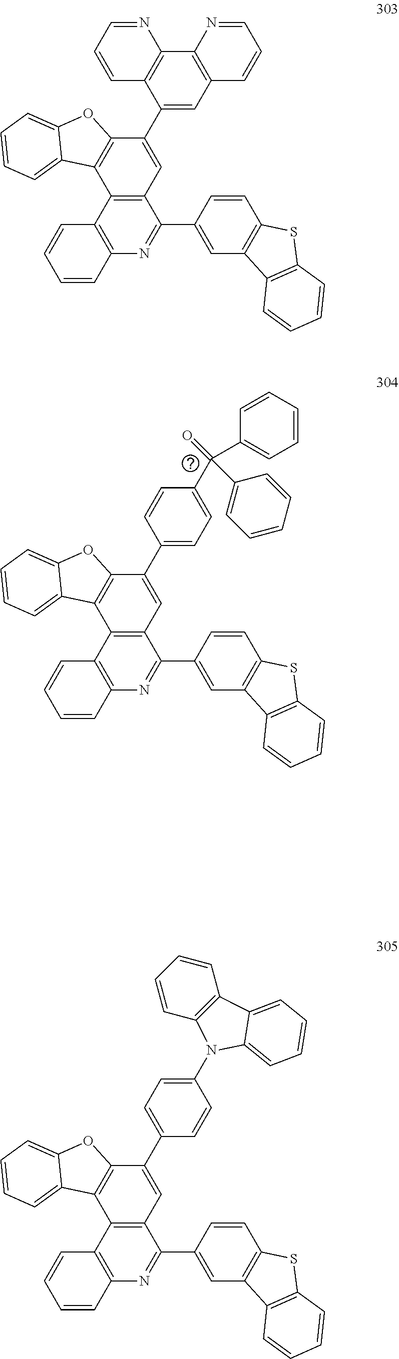

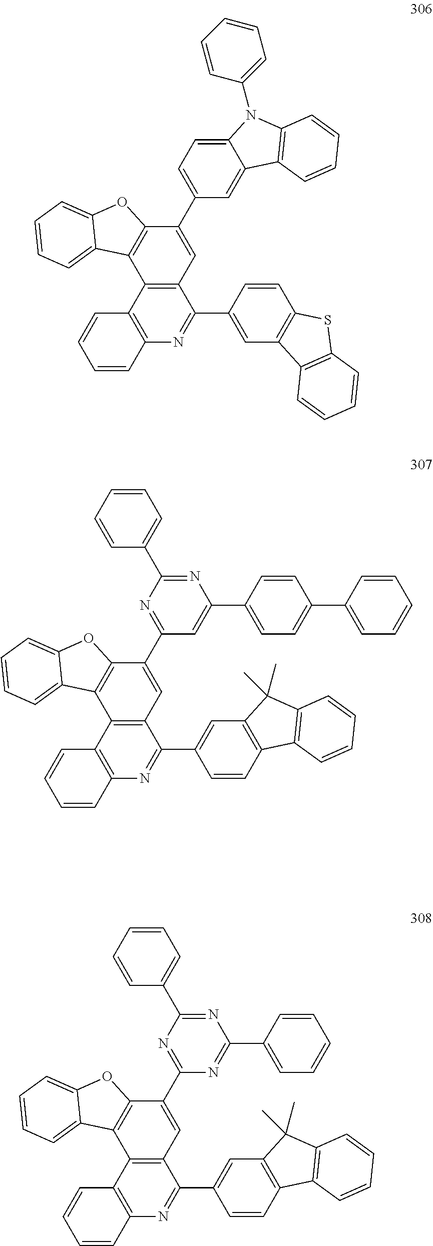 [SMILES: c1ccc(-n2c3ccccc3c3cc(-c4cc5c(-c6ccc7sc8ccccc8c7c6)nc6ccccc6c5c5c4oc4ccccc45)ccc32)cc1 ; CC1(C)c2ccccc2-c2ccc(-c3nc4ccccc4c4c3cc(-c3cc(-c5ccc(-c6ccccc6)cc5)nc(-c5ccccc5)n3)c3oc5ccccc5c34)cc21 ; CC1(C)c2ccccc2-c2ccc(-c3nc4ccccc4c4c3cc(-c3nc(-c5ccccc5)nc(-c5ccccc5)n3)c3oc5ccccc5c34)cc21]

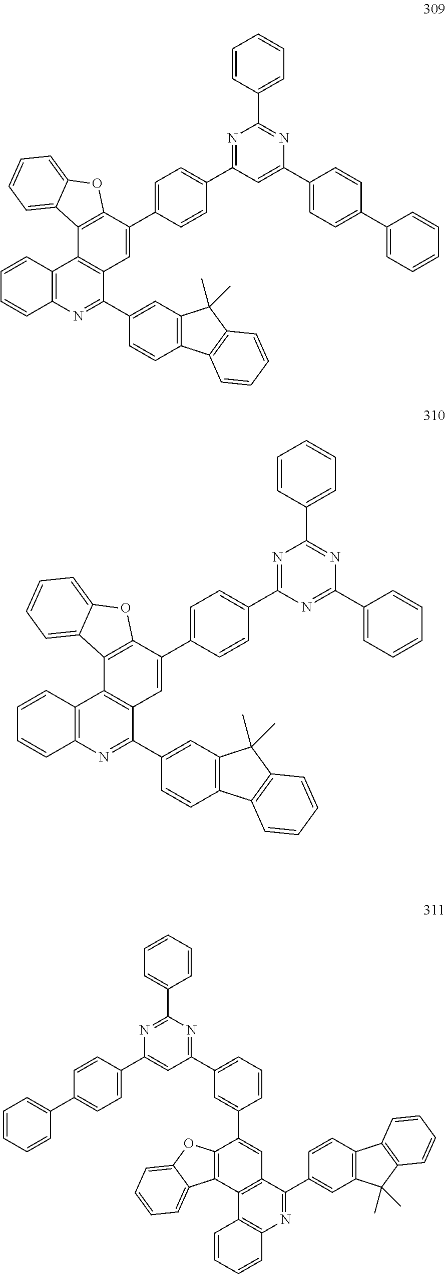 [SMILES: CC1(C)c2ccccc2-c2ccc(-c3nc4ccccc4c4c3cc(-c3ccc(-c5cc(-c6ccc(-c7ccccc7)cc6)nc(-c6ccccc6)n5)cc3)c3oc5ccccc5c34)cc21 ; CC1(C)c2ccccc2-c2ccc(-c3nc4ccccc4c4c3cc(-c3ccc(-c5nc(-c6ccccc6)nc(-c6ccccc6)n5)cc3)c3oc5ccccc5c34)cc21 ; CC1(C)c2ccccc2-c2ccc(-c3nc4ccccc4c4c3cc(-c3cccc(-c5cc(-c6ccc(-c7ccccc7)cc6)nc(-c6ccccc6)n5)c3)c3oc5ccccc5c34)cc21]

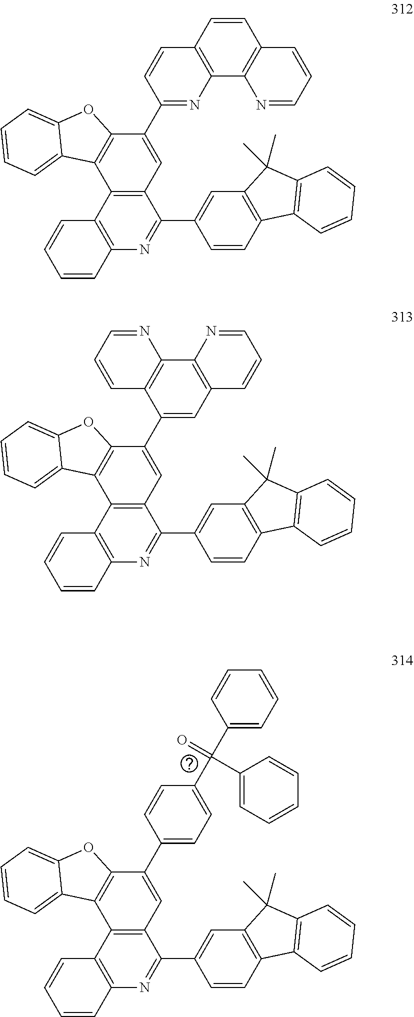

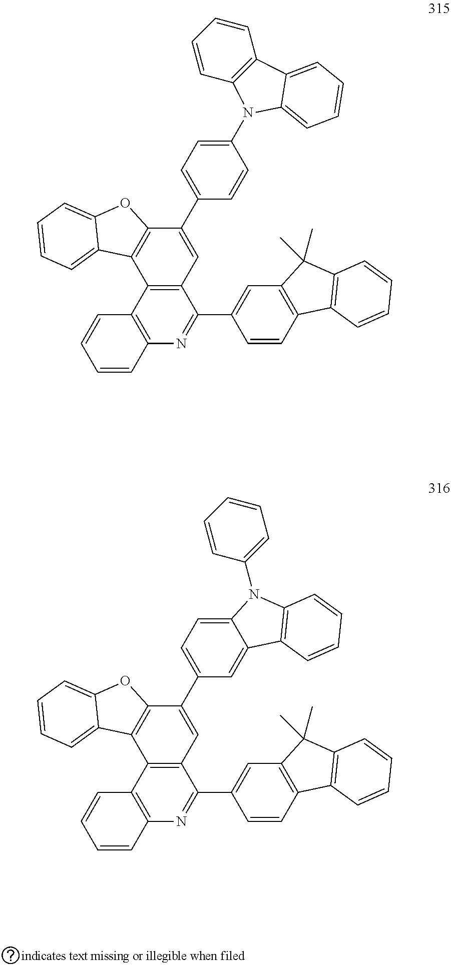 [SMILES: CC1(C)c2ccccc2-c2ccc(-c3nc4ccccc4c4c3cc(-c3ccc(-n5c6ccccc6c6ccccc65)cc3)c3oc5ccccc5c34)cc21 ; CC1(C)c2ccccc2-c2ccc(-c3nc4ccccc4c4c3cc(-c3ccc5c(c3)c3ccccc3n5-c3ccccc3)c3oc5ccccc5c34)cc21]

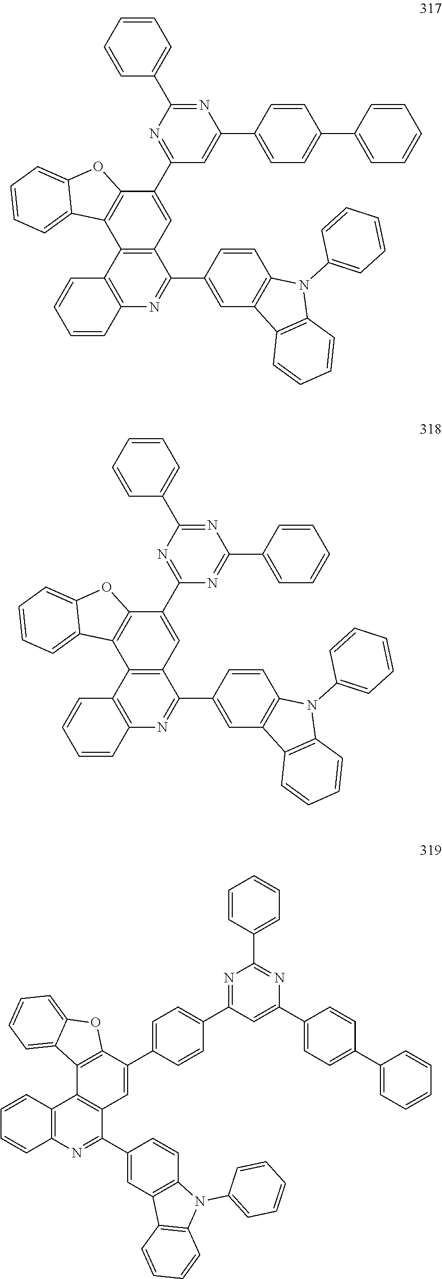 [SMILES: c1ccc(-c2ccc(-c3cc(-c4cc5c(-c6ccc7c(c6)c6ccccc6n7-c6ccccc6)nc6ccccc6c5c5c4oc4ccccc45)nc(-c4ccccc4)n3)cc2)cc1 ; c1ccc(-c2nc(-c3ccccc3)nc(-c3cc4c(-c5ccc6c(c5)c5ccccc5n6-c5ccccc5)nc5ccccc5c4c4c3oc3ccccc34)n2)cc1 ; c1ccc(-c2ccc(-c3cc(-c4ccc(-c5cc6c(-c7ccc8c(c7)c7ccccc7n8-c7ccccc7)nc7ccccc7c6c6c5oc5ccccc56)cc4)nc(-c4ccccc4)n3)cc2)cc1]

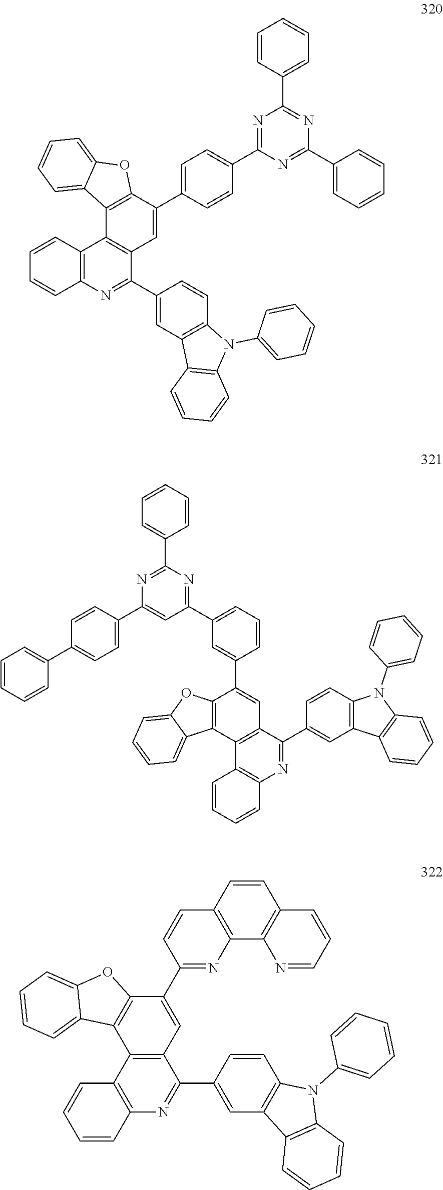 [SMILES: c1ccc(-c2nc(-c3ccccc3)nc(-c3ccc(-c4cc5c(-c6ccc7c(c6)c6ccccc6n7-c6ccccc6)nc6ccccc6c5c5c4oc4ccccc45)cc3)n2)cc1 ; c1ccc(-c2ccc(-c3cc(-c4cccc(-c5cc6c(-c7ccc8c(c7)c7ccccc7n8-c7ccccc7)nc7ccccc7c6c6c5oc5ccccc56)c4)nc(-c4ccccc4)n3)cc2)cc1 ; c1ccc(-n2c3ccccc3c3cc(-c4nc5ccccc5c5c4cc(-c4ccc6ccc7cccnc7c6n4)c4oc6ccccc6c45)ccc32)cc1]

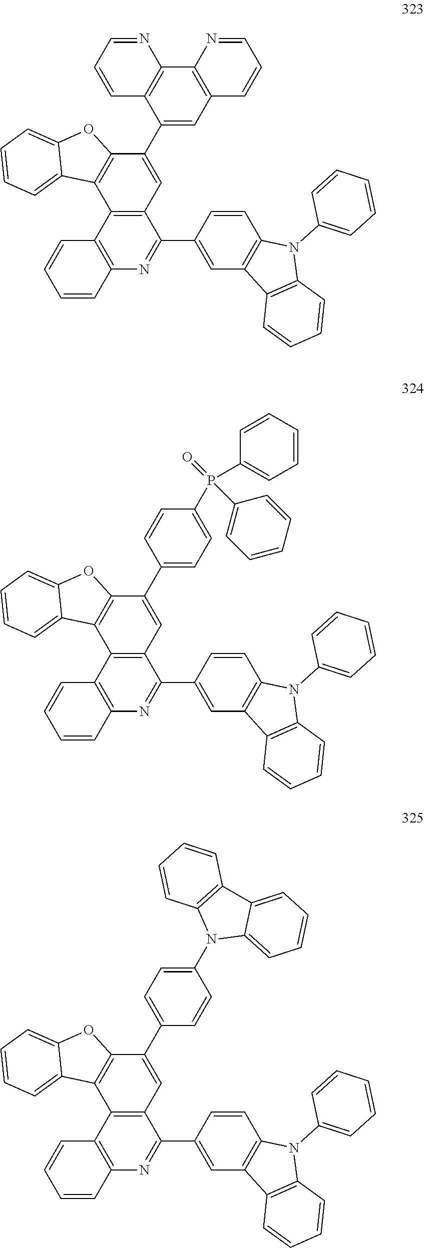 [SMILES: c1ccc(-n2c3ccccc3c3cc(-c4nc5ccccc5c5c4cc(-c4cc6cccnc6c6ncccc46)c4oc6ccccc6c45)ccc32)cc1 ; O=P(c1ccccc1)(c1ccccc1)c1ccc(-c2cc3c(-c4ccc5c(c4)c4ccccc4n5-c4ccccc4)nc4ccccc4c3c3c2oc2ccccc23)cc1 ; c1ccc(-n2c3ccccc3c3cc(-c4nc5ccccc5c5c4cc(-c4ccc(-n6c7ccccc7c7ccccc76)cc4)c4oc6ccccc6c45)ccc32)cc1]

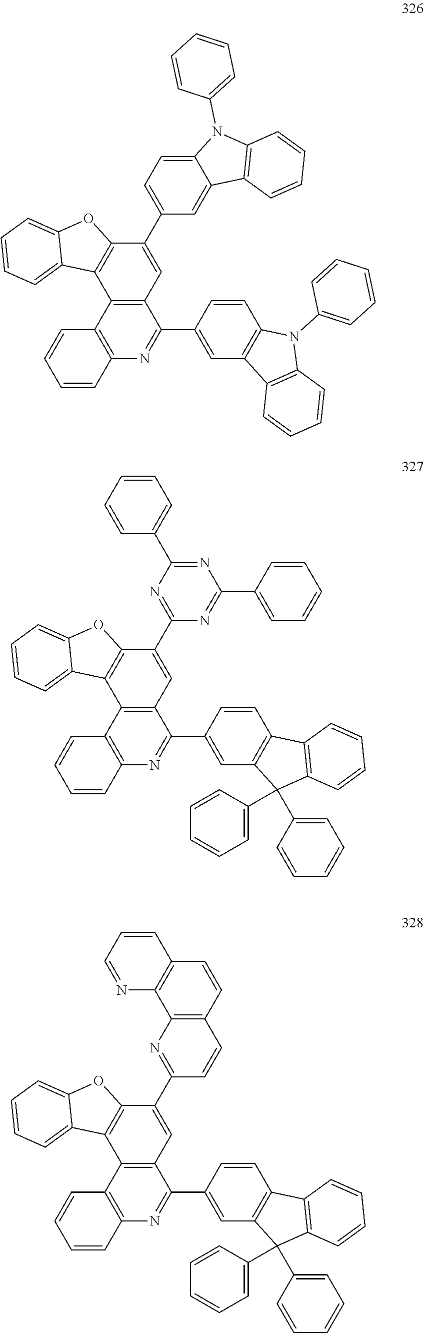 [SMILES: c1ccc(-n2c3ccccc3c3cc(-c4nc5ccccc5c5c4cc(-c4ccc6c(c4)c4ccccc4n6-c4ccccc4)c4oc6ccccc6c45)ccc32)cc1 ; c1ccc(-c2nc(-c3ccccc3)nc(-c3cc4c(-c5ccc6c(c5)C(c5ccccc5)(c5ccccc5)c5ccccc5-6)nc5ccccc5c4c4c3oc3ccccc34)n2)cc1 ; c1ccc(C2(c3ccccc3)c3ccccc3-c3ccc(-c4nc5ccccc5c5c4cc(-c4ccc6ccc7cccnc7c6n4)c4oc6ccccc6c45)cc32)cc1]

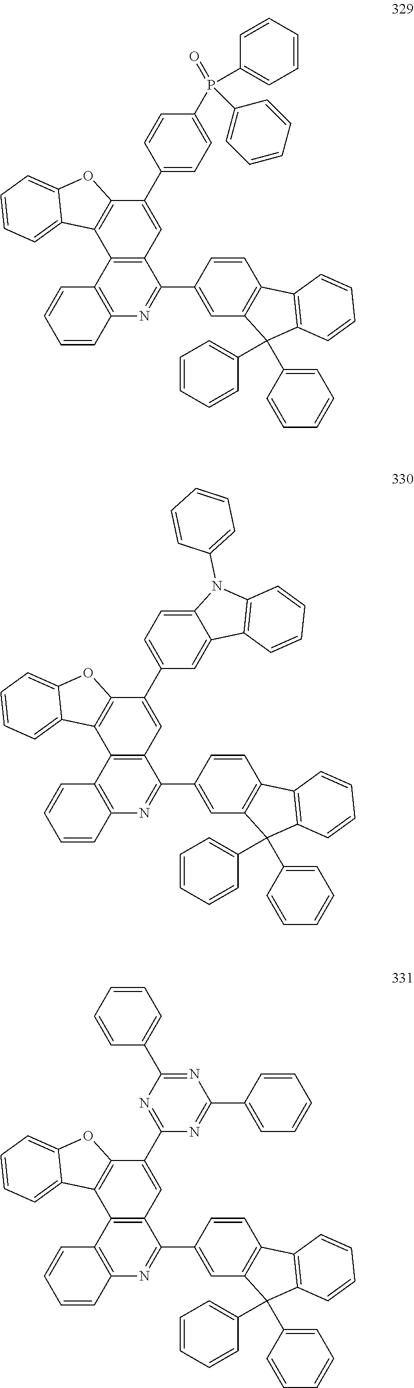 [SMILES: O=P(c1ccccc1)(c1ccccc1)c1ccc(-c2cc3c(-c4ccc5c(c4)C(c4ccccc4)(c4ccccc4)c4ccccc4-5)nc4ccccc4c3c3c2oc2ccccc23)cc1 ; c1ccc(-n2c3ccccc3c3cc(-c4cc5c(-c6ccc7c(c6)C(c6ccccc6)(c6ccccc6)c6ccccc6-7)nc6ccccc6c5c5c4oc4ccccc45)ccc32)cc1 ; c1ccc(-c2nc(-c3ccccc3)nc(-c3cc4c(-c5ccc6c(c5)C(c5ccccc5)(c5ccccc5)c5ccccc5-6)nc5ccccc5c4c4c3oc3ccccc34)n2)cc1]

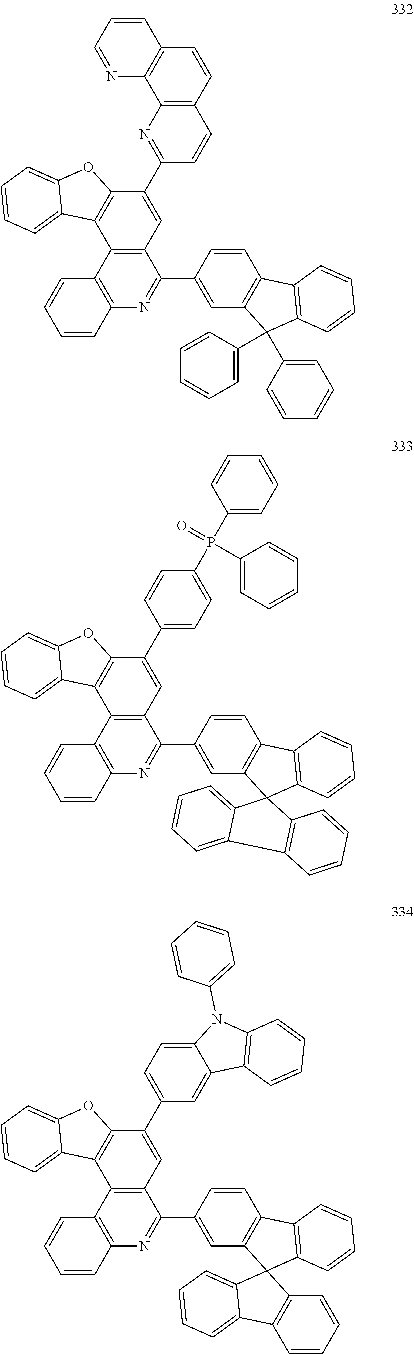

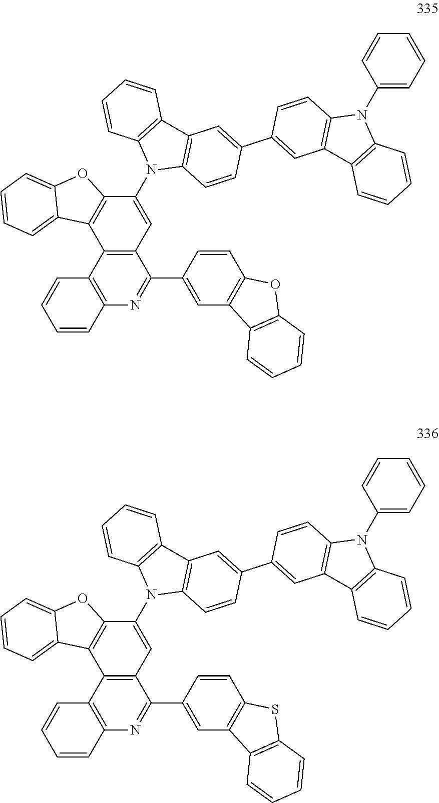 [SMILES: c1ccc(-n2c3ccccc3c3cc(-c4ccc5c(c4)c4ccccc4n5-c4cc5c(-c6ccc7oc8ccccc8c7c6)nc6ccccc6c5c5c4oc4ccccc45)ccc32)cc1 ; c1ccc(-n2c3ccccc3c3cc(-c4ccc5c(c4)c4ccccc4n5-c4cc5c(-c6ccc7sc8ccccc8c7c6)nc6ccccc6c5c5c4oc4ccccc45)ccc32)cc1]

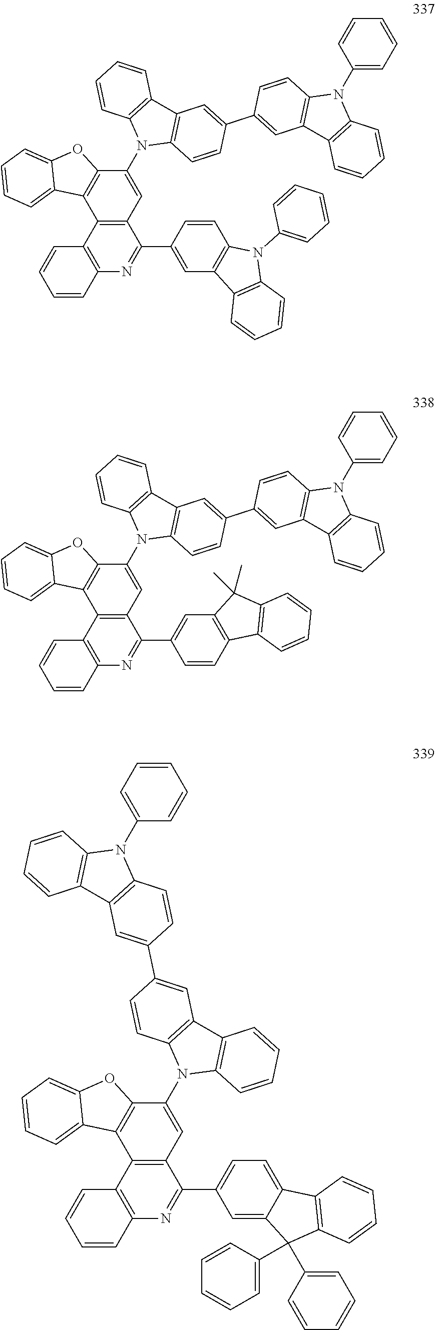 [SMILES: c1ccc(-n2c3ccccc3c3cc(-c4ccc5c(c4)c4ccccc4n5-c4cc5c(-c6ccc7c(c6)c6ccccc6n7-c6ccccc6)nc6ccccc6c5c5c4oc4ccccc45)ccc32)cc1 ; CC1(C)c2ccccc2-c2ccc(-c3nc4ccccc4c4c3cc(-n3c5ccccc5c5cc(-c6ccc7c(c6)c6ccccc6n7-c6ccccc6)ccc53)c3oc5ccccc5c34)cc21 ; c1ccc(-n2c3ccccc3c3cc(-c4ccc5c(c4)c4ccccc4n5-c4cc5c(-c6ccc7c(c6)C(c6ccccc6)(c6ccccc6)c6ccccc6-7)nc6ccccc6c5c5c4oc4ccccc45)ccc32)cc1]

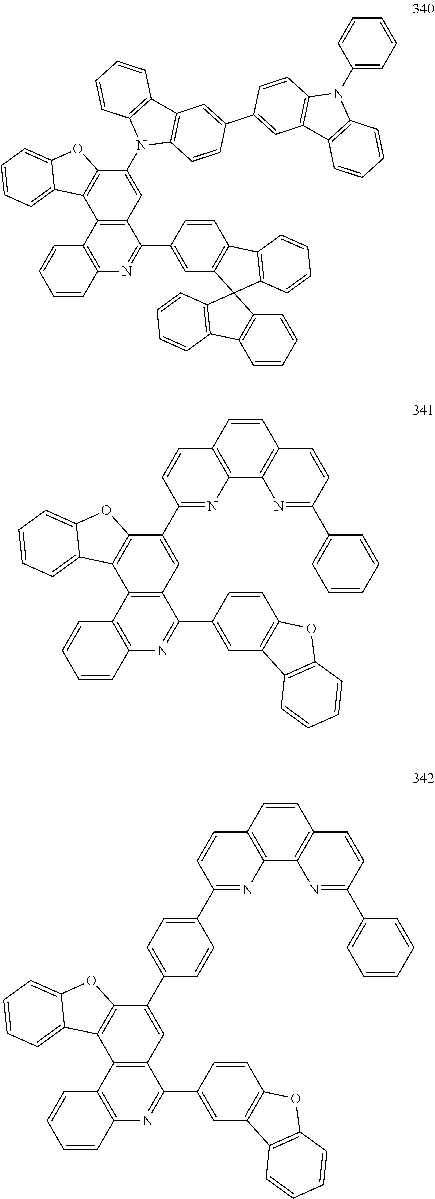 [SMILES: c1ccc(-n2c3ccccc3c3cc(-c4ccc5c(c4)c4ccccc4n5-c4cc5c(-c6ccc7c(c6)C6(c8ccccc8-c8ccccc86)c6ccccc6-7)nc6ccccc6c5c5c4oc4ccccc45)ccc32)cc1 ; c1ccc(-c2ccc3ccc4ccc(-c5cc6c(-c7ccc8oc9ccccc9c8c7)nc7ccccc7c6c6c5oc5ccccc56)nc4c3n2)cc1 ; c1ccc(-c2ccc3ccc4ccc(-c5ccc(-c6cc7c(-c8ccc9oc%10ccccc%10c9c8)nc8ccccc8c7c7c6oc6ccccc67)cc5)nc4c3n2)cc1]

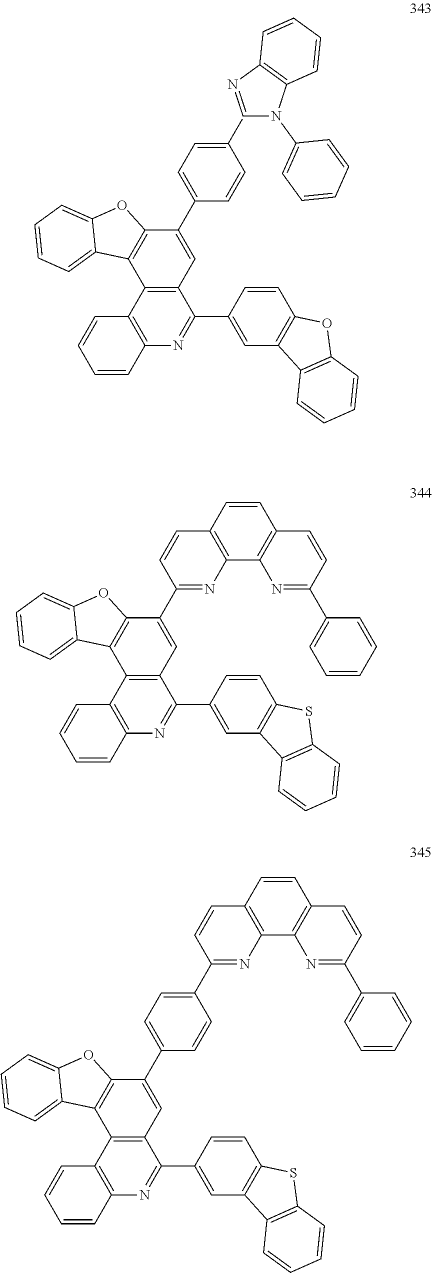

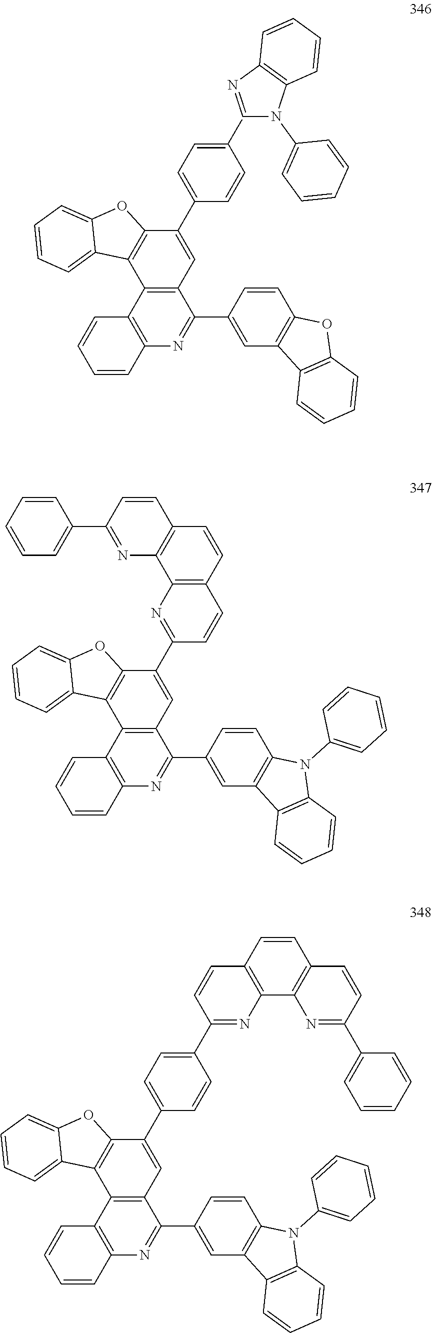

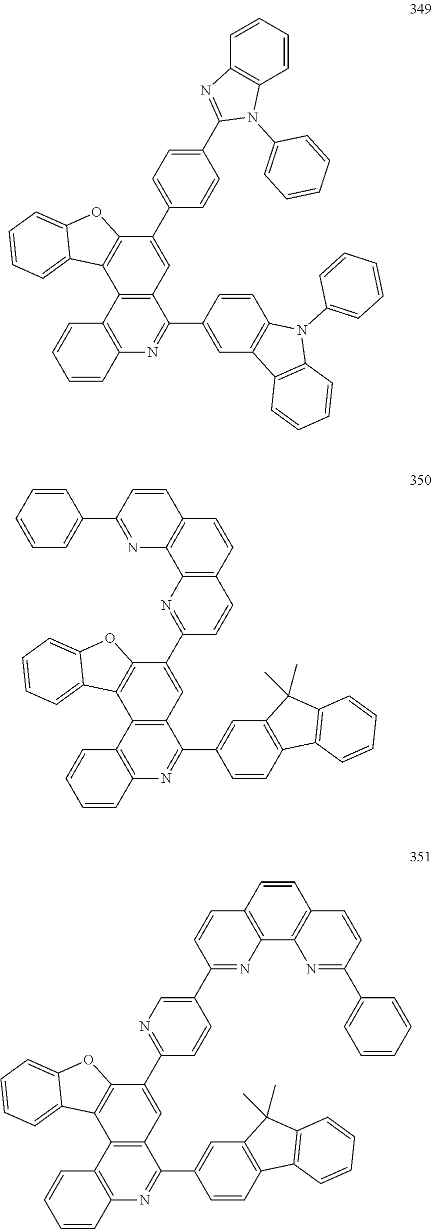 [SMILES: c1ccc(-n2c(-c3ccc(-c4cc5c(-c6ccc7c(c6)c6ccccc6n7-c6ccccc6)nc6ccccc6c5c5c4oc4ccccc45)cc3)nc3ccccc32)cc1 ; CC1(C)c2ccccc2-c2ccc(-c3nc4ccccc4c4c3cc(-c3ccc5ccc6ccc(-c7ccccc7)nc6c5n3)c3oc5ccccc5c34)cc21 ; CC1(C)c2ccccc2-c2ccc(-c3nc4ccccc4c4c3cc(-c3ccc(-c5ccc6ccc7ccc(-c8ccccc8)nc7c6n5)cn3)c3oc5ccccc5c34)cc21]

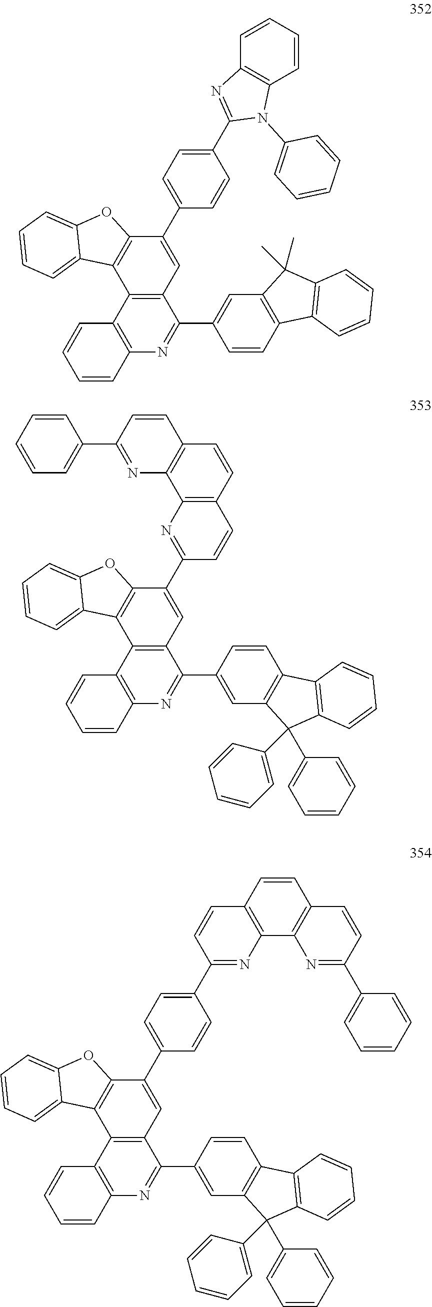

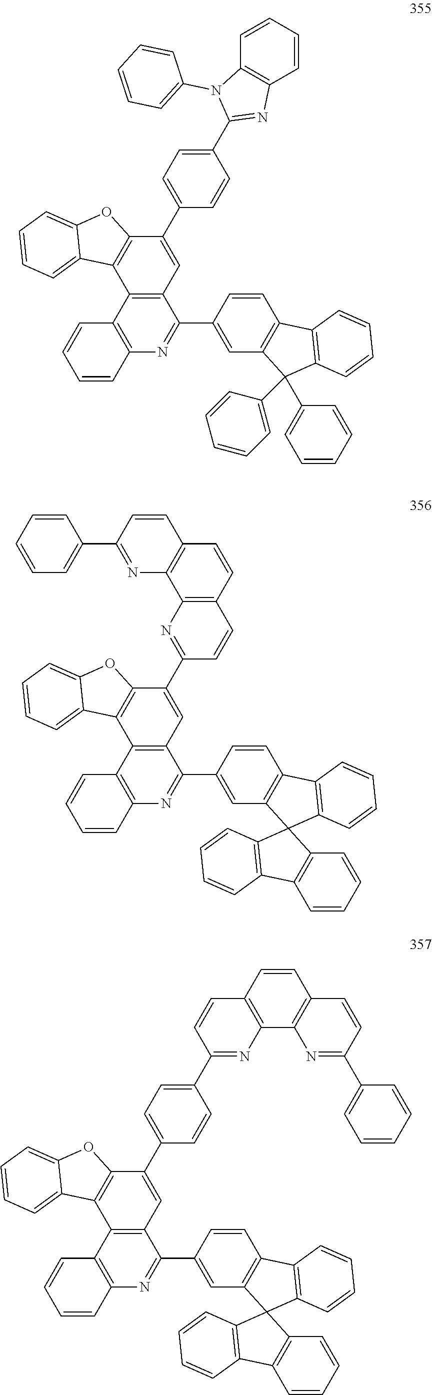 [SMILES: c1ccc(-n2c(-c3ccc(-c4cc5c(-c6ccc7c(c6)C(c6ccccc6)(c6ccccc6)c6ccccc6-7)nc6ccccc6c5c5c4oc4ccccc45)cc3)nc3ccccc32)cc1 ; c1ccc(-c2ccc3ccc4ccc(-c5cc6c(-c7ccc8c(c7)C7(c9ccccc9-c9ccccc97)c7ccccc7-8)nc7ccccc7c6c6c5oc5ccccc56)nc4c3n2)cc1 ; c1ccc(-c2ccc3ccc4ccc(-c5ccc(-c6cc7c(-c8ccc9c(c8)C8(c%10ccccc%10-c%10ccccc%108)c8ccccc8-9)nc8ccccc8c7c7c6oc6ccccc67)cc5)nc4c3n2)cc1]

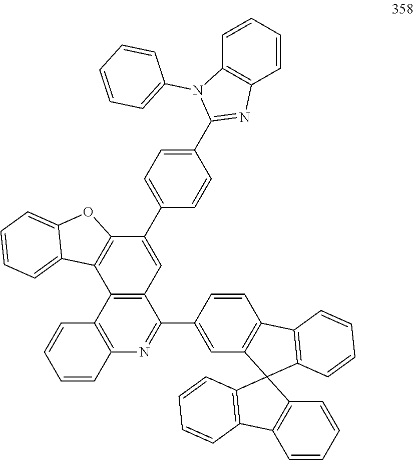 [SMILES: c1ccc(-n2c(-c3ccc(-c4cc5c(-c6ccc7c(c6)C6(c8ccccc8-c8ccccc86)c6ccccc6-7)nc6ccccc6c5c5c4oc4ccccc45)cc3)nc3ccccc32)cc1]

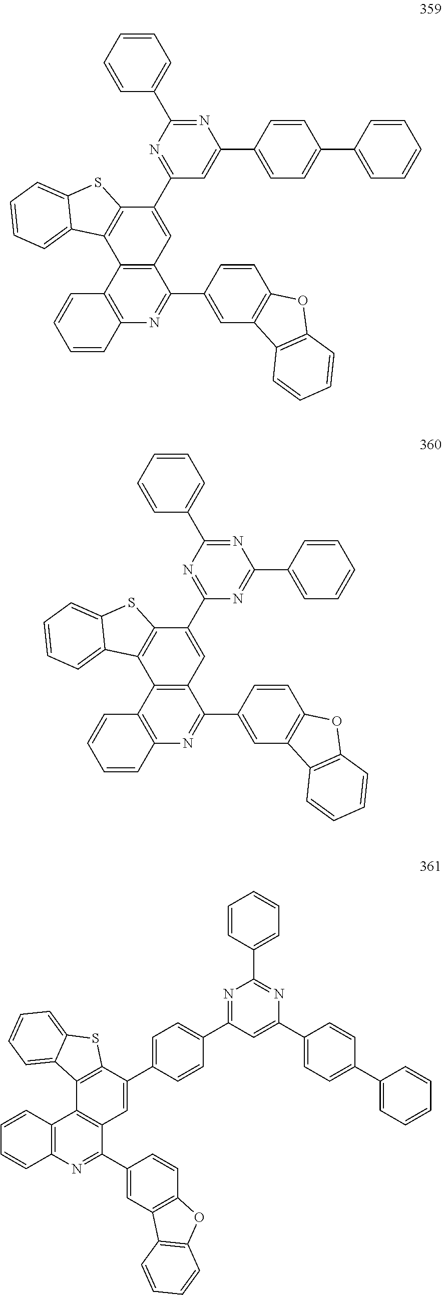 [SMILES: c1ccc(-c2ccc(-c3cc(-c4cc5c(-c6ccc7oc8ccccc8c7c6)nc6ccccc6c5c5c4sc4ccccc45)nc(-c4ccccc4)n3)cc2)cc1 ; c1ccc(-c2nc(-c3ccccc3)nc(-c3cc4c(-c5ccc6oc7ccccc7c6c5)nc5ccccc5c4c4c3sc3ccccc34)n2)cc1 ; c1ccc(-c2ccc(-c3cc(-c4ccc(-c5cc6c(-c7ccc8oc9ccccc9c8c7)nc7ccccc7c6c6c5sc5ccccc56)cc4)nc(-c4ccccc4)n3)cc2)cc1]

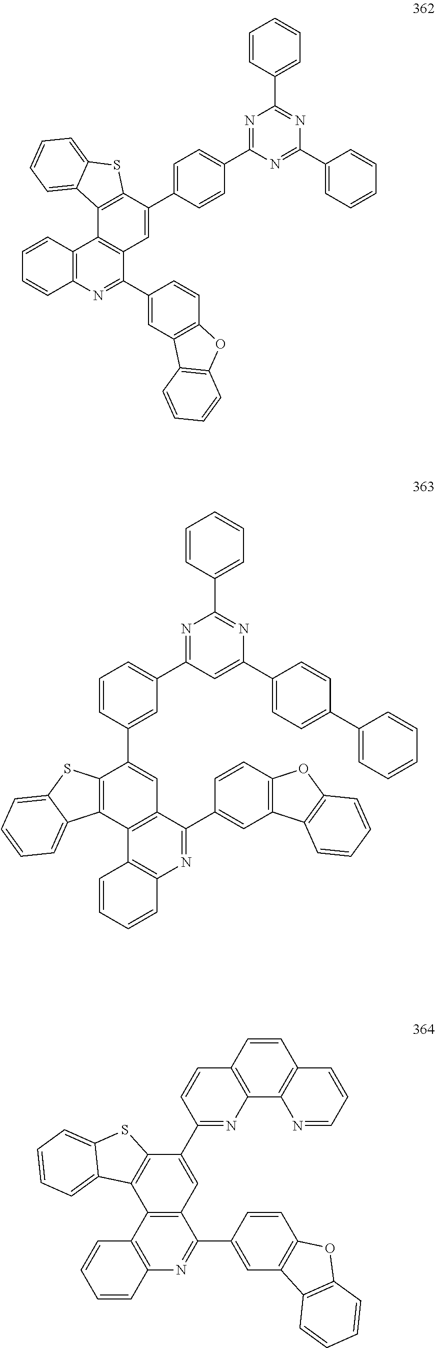 [SMILES: c1ccc(-c2nc(-c3ccccc3)nc(-c3ccc(-c4cc5c(-c6ccc7oc8ccccc8c7c6)nc6ccccc6c5c5c4sc4ccccc45)cc3)n2)cc1 ; c1ccc(-c2ccc(-c3cc(-c4cccc(-c5cc6c(-c7ccc8oc9ccccc9c8c7)nc7ccccc7c6c6c5sc5ccccc56)c4)nc(-c4ccccc4)n3)cc2)cc1 ; c1cnc2c(c1)ccc1ccc(-c3cc4c(-c5ccc6oc7ccccc7c6c5)nc5ccccc5c4c4c3sc3ccccc34)nc12]

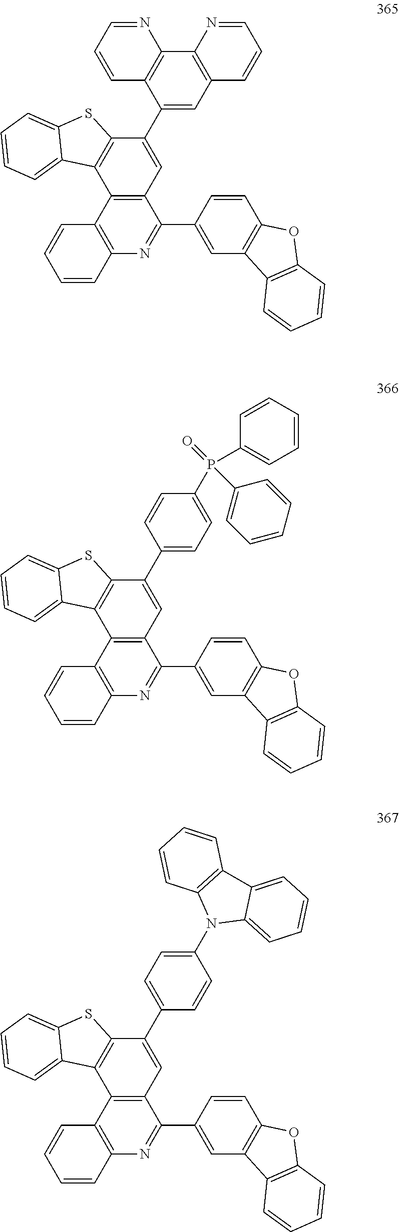 [SMILES: c1cnc2c(c1)cc(-c1cc3c(-c4ccc5oc6ccccc6c5c4)nc4ccccc4c3c3c1sc1ccccc13)c1cccnc12 ; O=P(c1ccccc1)(c1ccccc1)c1ccc(-c2cc3c(-c4ccc5oc6ccccc6c5c4)nc4ccccc4c3c3c2sc2ccccc23)cc1 ; c1ccc2c(c1)nc(-c1ccc3oc4ccccc4c3c1)c1cc(-c3ccc(-n4c5ccccc5c5ccccc54)cc3)c3sc4ccccc4c3c12]

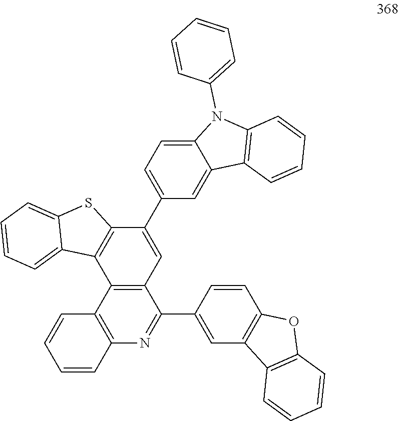 [SMILES: c1ccc(-n2c3ccccc3c3cc(-c4cc5c(-c6ccc7oc8ccccc8c7c6)nc6ccccc6c5c5c4sc4ccccc45)ccc32)cc1]

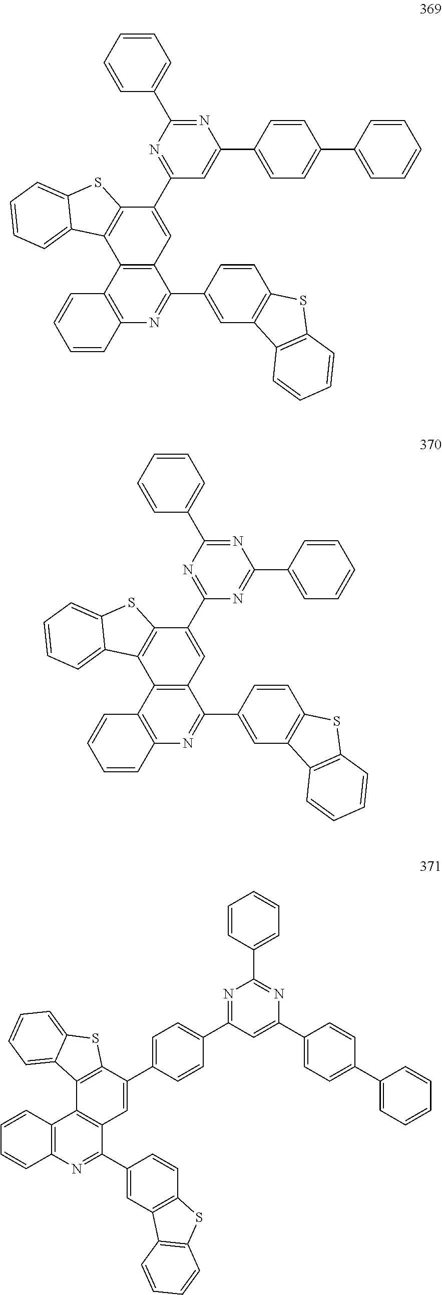 [SMILES: c1ccc(-c2ccc(-c3cc(-c4cc5c(-c6ccc7sc8ccccc8c7c6)nc6ccccc6c5c5c4sc4ccccc45)nc(-c4ccccc4)n3)cc2)cc1 ; c1ccc(-c2nc(-c3ccccc3)nc(-c3cc4c(-c5ccc6sc7ccccc7c6c5)nc5ccccc5c4c4c3sc3ccccc34)n2)cc1 ; c1ccc(-c2ccc(-c3cc(-c4ccc(-c5cc6c(-c7ccc8sc9ccccc9c8c7)nc7ccccc7c6c6c5sc5ccccc56)cc4)nc(-c4ccccc4)n3)cc2)cc1]

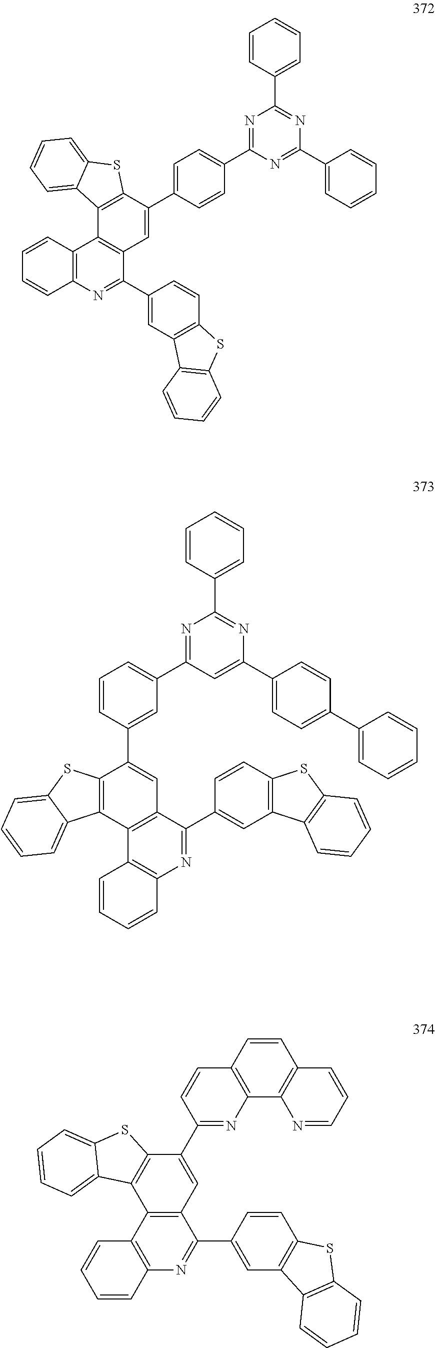 [SMILES: c1ccc(-c2nc(-c3ccccc3)nc(-c3ccc(-c4cc5c(-c6ccc7sc8ccccc8c7c6)nc6ccccc6c5c5c4sc4ccccc45)cc3)n2)cc1 ; c1ccc(-c2ccc(-c3cc(-c4cccc(-c5cc6c(-c7ccc8sc9ccccc9c8c7)nc7ccccc7c6c6c5sc5ccccc56)c4)nc(-c4ccccc4)n3)cc2)cc1 ; c1cnc2c(c1)ccc1ccc(-c3cc4c(-c5ccc6sc7ccccc7c6c5)nc5ccccc5c4c4c3sc3ccccc34)nc12]

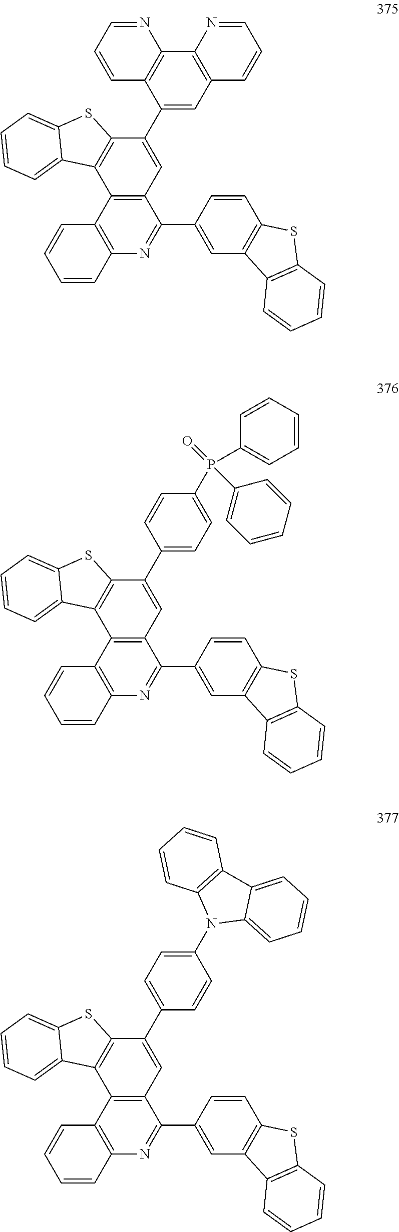 [SMILES: c1cnc2c(c1)cc(-c1cc3c(-c4ccc5sc6ccccc6c5c4)nc4ccccc4c3c3c1sc1ccccc13)c1cccnc12 ; O=P(c1ccccc1)(c1ccccc1)c1ccc(-c2cc3c(-c4ccc5sc6ccccc6c5c4)nc4ccccc4c3c3c2sc2ccccc23)cc1 ; c1ccc2c(c1)nc(-c1ccc3sc4ccccc4c3c1)c1cc(-c3ccc(-n4c5ccccc5c5ccccc54)cc3)c3sc4ccccc4c3c12]

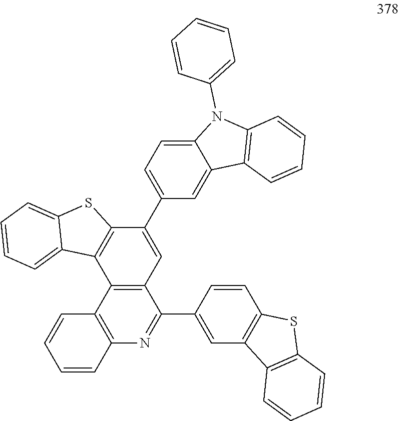 [SMILES: c1ccc(-n2c3ccccc3c3cc(-c4cc5c(-c6ccc7sc8ccccc8c7c6)nc6ccccc6c5c5c4sc4ccccc45)ccc32)cc1]

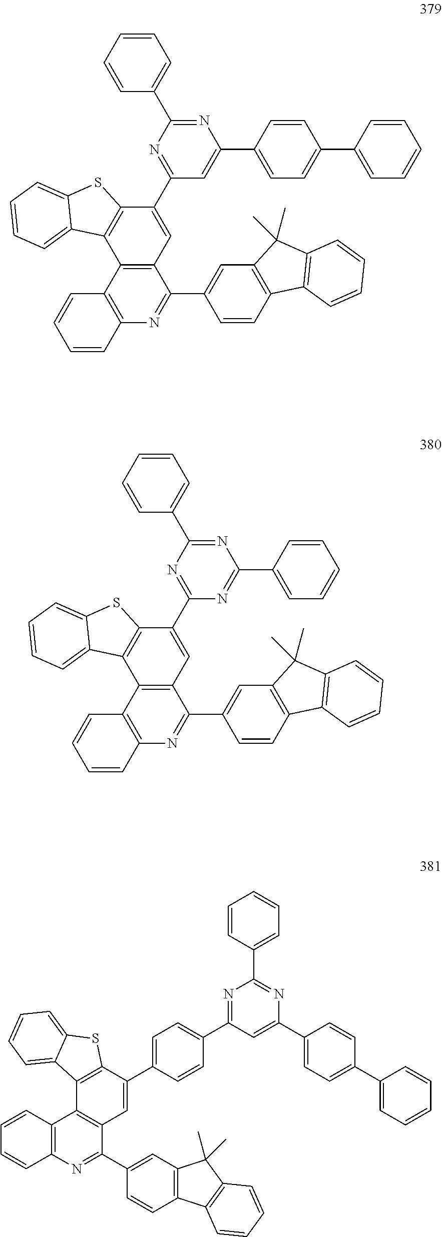 [SMILES: CC1(C)c2ccccc2-c2ccc(-c3nc4ccccc4c4c3cc(-c3cc(-c5ccc(-c6ccccc6)cc5)nc(-c5ccccc5)n3)c3sc5ccccc5c34)cc21 ; CC1(C)c2ccccc2-c2ccc(-c3nc4ccccc4c4c3cc(-c3nc(-c5ccccc5)nc(-c5ccccc5)n3)c3sc5ccccc5c34)cc21 ; CC1(C)c2ccccc2-c2ccc(-c3nc4ccccc4c4c3cc(-c3ccc(-c5cc(-c6ccc(-c7ccccc7)cc6)nc(-c6ccccc6)n5)cc3)c3sc5ccccc5c34)cc21]

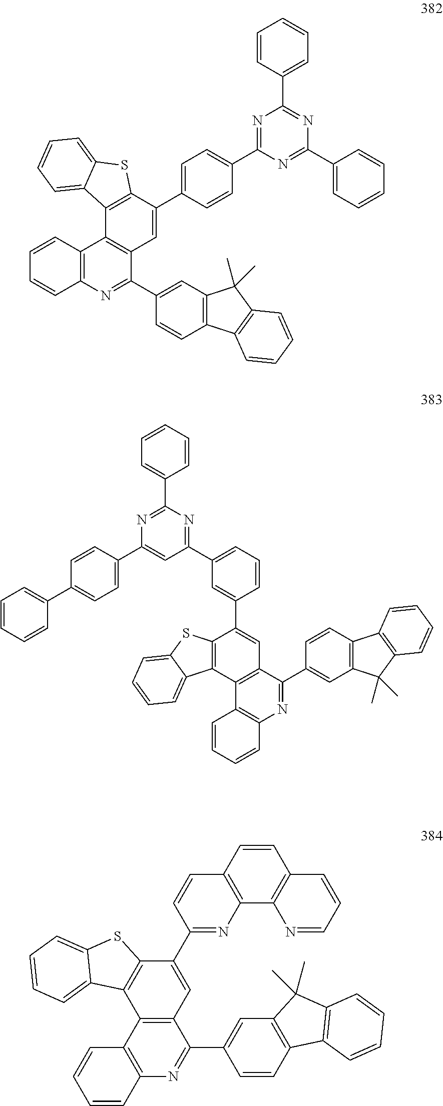 [SMILES: CC1(C)c2ccccc2-c2ccc(-c3nc4ccccc4c4c3cc(-c3ccc(-c5nc(-c6ccccc6)nc(-c6ccccc6)n5)cc3)c3sc5ccccc5c34)cc21 ; CC1(C)c2ccccc2-c2ccc(-c3nc4ccccc4c4c3cc(-c3cccc(-c5cc(-c6ccc(-c7ccccc7)cc6)nc(-c6ccccc6)n5)c3)c3sc5ccccc5c34)cc21 ; CC1(C)c2ccccc2-c2ccc(-c3nc4ccccc4c4c3cc(-c3ccc5ccc6cccnc6c5n3)c3sc5ccccc5c34)cc21]

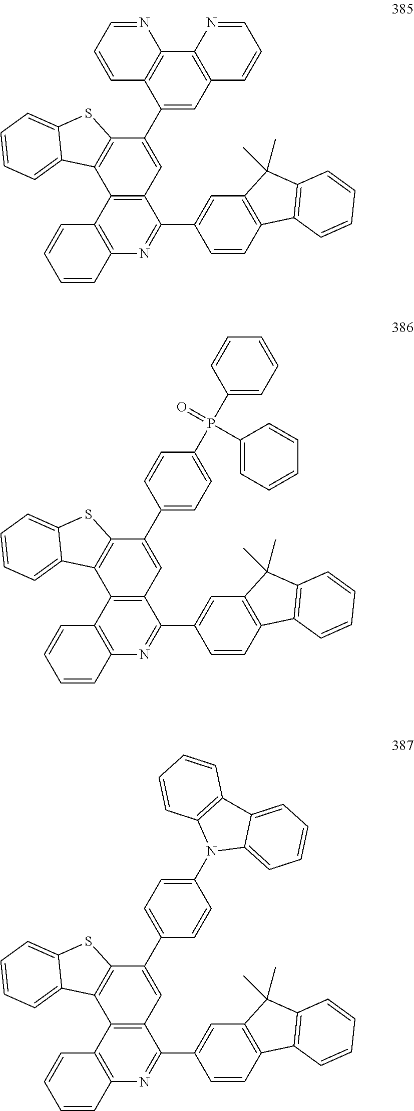 [SMILES: CC1(C)c2ccccc2-c2ccc(-c3nc4ccccc4c4c3cc(-c3cc5cccnc5c5ncccc35)c3sc5ccccc5c34)cc21 ; CC1(C)c2ccccc2-c2ccc(-c3nc4ccccc4c4c3cc(-c3ccc(P(=O)(c5ccccc5)c5ccccc5)cc3)c3sc5ccccc5c34)cc21 ; CC1(C)c2ccccc2-c2ccc(-c3nc4ccccc4c4c3cc(-c3ccc(-n5c6ccccc6c6ccccc65)cc3)c3sc5ccccc5c34)cc21]

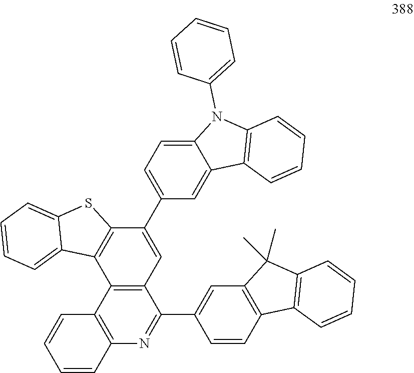 [SMILES: CC1(C)c2ccccc2-c2ccc(-c3nc4ccccc4c4c3cc(-c3ccc5c(c3)c3ccccc3n5-c3ccccc3)c3sc5ccccc5c34)cc21]

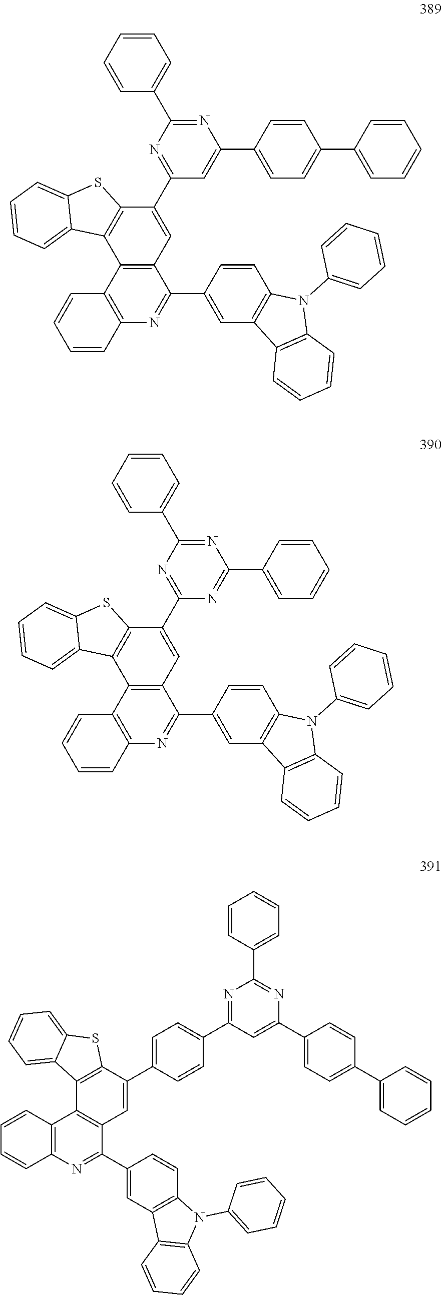 [SMILES: c1ccc(-c2ccc(-c3cc(-c4cc5c(-c6ccc7c(c6)c6ccccc6n7-c6ccccc6)nc6ccccc6c5c5c4sc4ccccc45)nc(-c4ccccc4)n3)cc2)cc1 ; c1ccc(-c2nc(-c3ccccc3)nc(-c3cc4c(-c5ccc6c(c5)c5ccccc5n6-c5ccccc5)nc5ccccc5c4c4c3sc3ccccc34)n2)cc1 ; c1ccc(-c2ccc(-c3cc(-c4ccc(-c5cc6c(-c7ccc8c(c7)c7ccccc7n8-c7ccccc7)nc7ccccc7c6c6c5sc5ccccc56)cc4)nc(-c4ccccc4)n3)cc2)cc1]

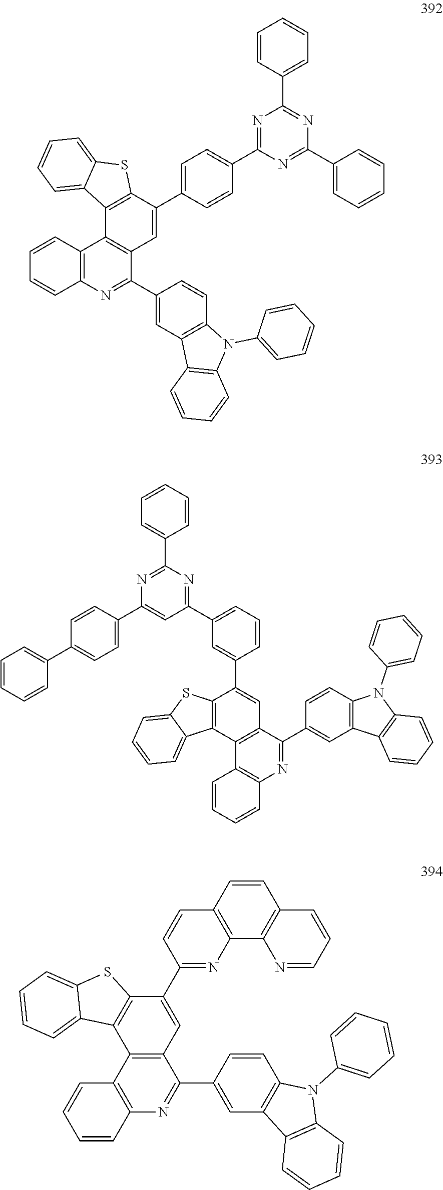 [SMILES: c1ccc(-c2nc(-c3ccccc3)nc(-c3ccc(-c4cc5c(-c6ccc7c(c6)c6ccccc6n7-c6ccccc6)nc6ccccc6c5c5c4sc4ccccc45)cc3)n2)cc1 ; c1ccc(-c2ccc(-c3cc(-c4cccc(-c5cc6c(-c7ccc8c(c7)c7ccccc7n8-c7ccccc7)nc7ccccc7c6c6c5sc5ccccc56)c4)nc(-c4ccccc4)n3)cc2)cc1 ; c1ccc(-n2c3ccccc3c3cc(-c4nc5ccccc5c5c4cc(-c4ccc6ccc7cccnc7c6n4)c4sc6ccccc6c45)ccc32)cc1]

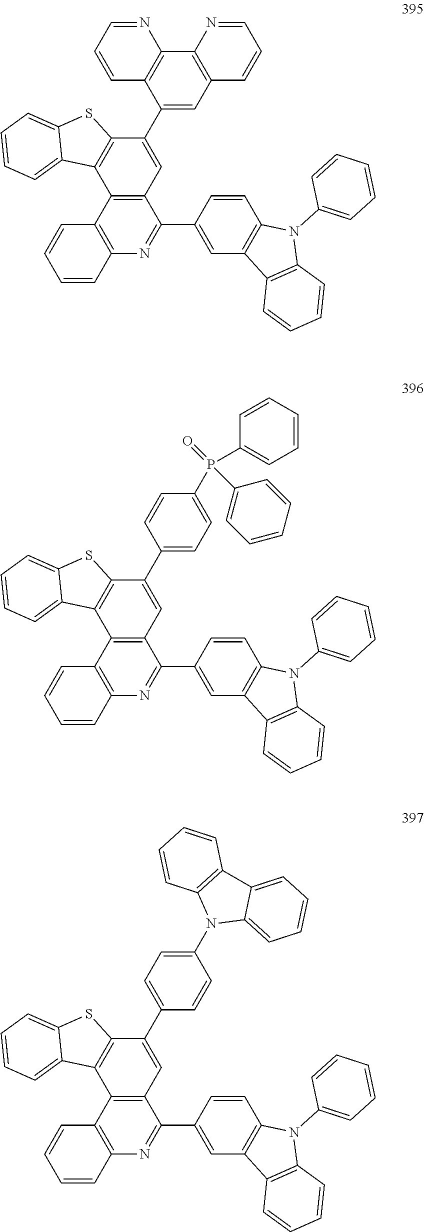 [SMILES: c1ccc(-n2c3ccccc3c3cc(-c4nc5ccccc5c5c4cc(-c4cc6cccnc6c6ncccc46)c4sc6ccccc6c45)ccc32)cc1 ; O=P(c1ccccc1)(c1ccccc1)c1ccc(-c2cc3c(-c4ccc5c(c4)c4ccccc4n5-c4ccccc4)nc4ccccc4c3c3c2sc2ccccc23)cc1 ; c1ccc(-n2c3ccccc3c3cc(-c4nc5ccccc5c5c4cc(-c4ccc(-n6c7ccccc7c7ccccc76)cc4)c4sc6ccccc6c45)ccc32)cc1]

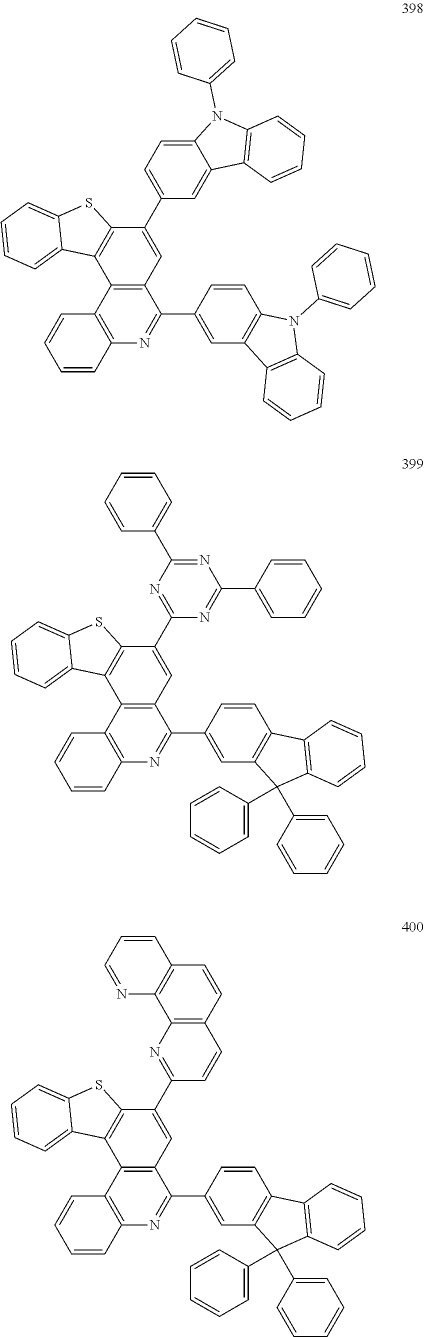 [SMILES: c1ccc(-n2c3ccccc3c3cc(-c4nc5ccccc5c5c4cc(-c4ccc6c(c4)c4ccccc4n6-c4ccccc4)c4sc6ccccc6c45)ccc32)cc1 ; c1ccc(-c2nc(-c3ccccc3)nc(-c3cc4c(-c5ccc6c(c5)C(c5ccccc5)(c5ccccc5)c5ccccc5-6)nc5ccccc5c4c4c3sc3ccccc34)n2)cc1 ; c1ccc(C2(c3ccccc3)c3ccccc3-c3ccc(-c4nc5ccccc5c5c4cc(-c4ccc6ccc7cccnc7c6n4)c4sc6ccccc6c45)cc32)cc1]

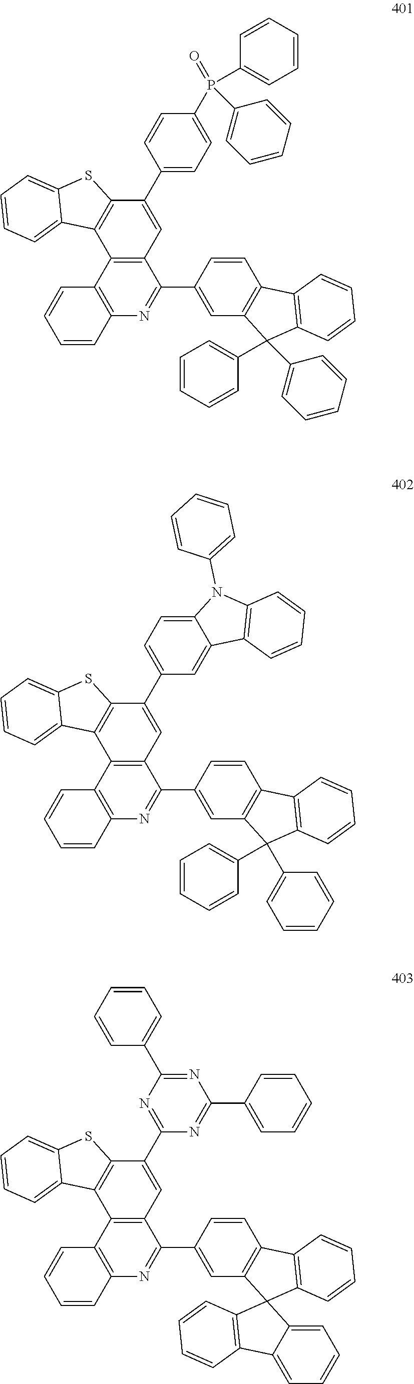 [SMILES: O=P(c1ccccc1)(c1ccccc1)c1ccc(-c2cc3c(-c4ccc5c(c4)C(c4ccccc4)(c4ccccc4)c4ccccc4-5)nc4ccccc4c3c3c2sc2ccccc23)cc1 ; c1ccc(-n2c3ccccc3c3cc(-c4cc5c(-c6ccc7c(c6)C(c6ccccc6)(c6ccccc6)c6ccccc6-7)nc6ccccc6c5c5c4sc4ccccc45)ccc32)cc1 ; c1ccc(-c2nc(-c3ccccc3)nc(-c3cc4c(-c5ccc6c(c5)C5(c7ccccc7-c7ccccc75)c5ccccc5-6)nc5ccccc5c4c4c3sc3ccccc34)n2)cc1]

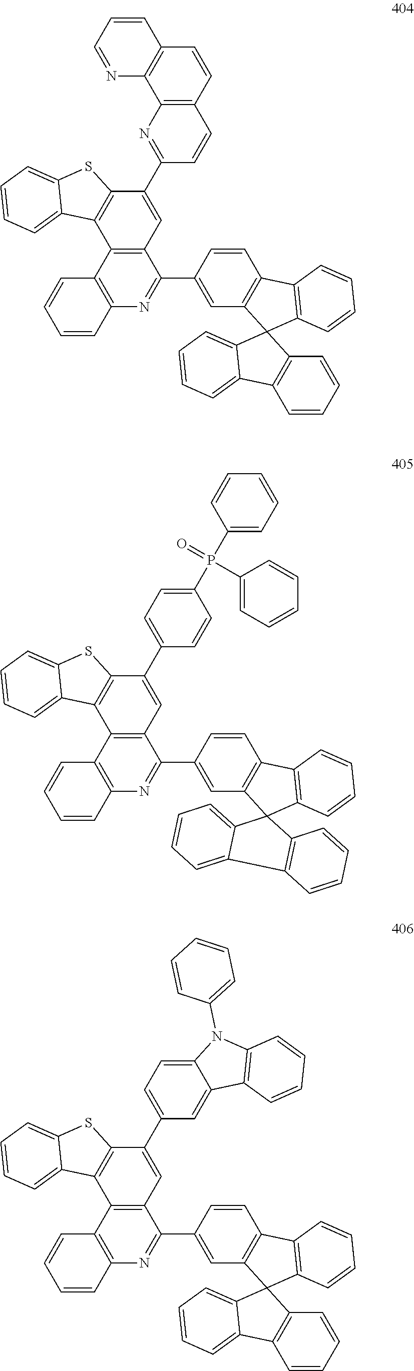 [SMILES: c1ccc2c(c1)-c1ccccc1C21c2ccccc2-c2ccc(-c3nc4ccccc4c4c3cc(-c3ccc5ccc6cccnc6c5n3)c3sc5ccccc5c34)cc21 ; O=P(c1ccccc1)(c1ccccc1)c1ccc(-c2cc3c(-c4ccc5c(c4)C4(c6ccccc6-c6ccccc64)c4ccccc4-5)nc4ccccc4c3c3c2sc2ccccc23)cc1 ; c1ccc(-n2c3ccccc3c3cc(-c4cc5c(-c6ccc7c(c6)C6(c8ccccc8-c8ccccc86)c6ccccc6-7)nc6ccccc6c5c5c4sc4ccccc45)ccc32)cc1]

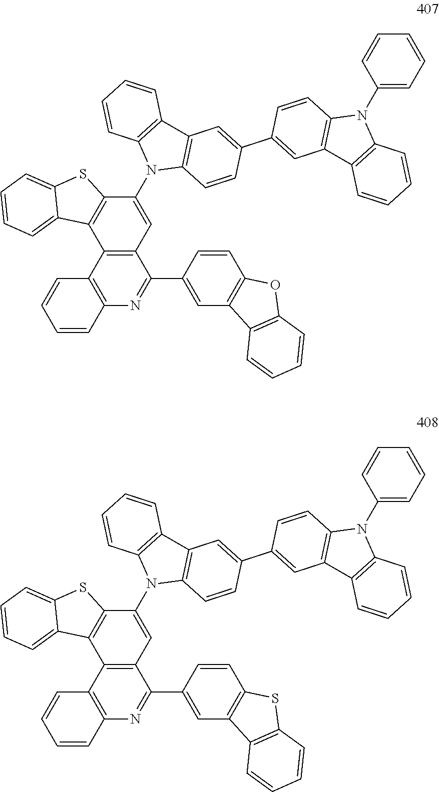 [SMILES: c1ccc(-n2c3ccccc3c3cc(-c4ccc5c(c4)c4ccccc4n5-c4cc5c(-c6ccc7oc8ccccc8c7c6)nc6ccccc6c5c5c4sc4ccccc45)ccc32)cc1 ; c1ccc(-n2c3ccccc3c3cc(-c4ccc5c(c4)c4ccccc4n5-c4cc5c(-c6ccc7sc8ccccc8c7c6)nc6ccccc6c5c5c4sc4ccccc45)ccc32)cc1]

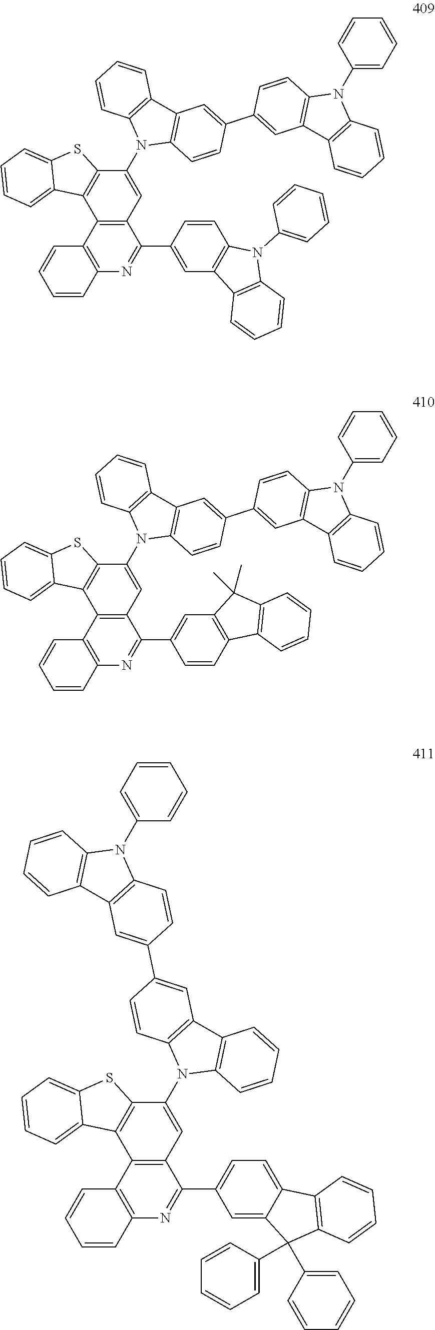 [SMILES: c1ccc(-n2c3ccccc3c3cc(-c4ccc5c(c4)c4ccccc4n5-c4cc5c(-c6ccc7c(c6)c6ccccc6n7-c6ccccc6)nc6ccccc6c5c5c4sc4ccccc45)ccc32)cc1 ; CC1(C)c2ccccc2-c2ccc(-c3nc4ccccc4c4c3cc(-n3c5ccccc5c5cc(-c6ccc7c(c6)c6ccccc6n7-c6ccccc6)ccc53)c3sc5ccccc5c34)cc21 ; c1ccc(-n2c3ccccc3c3cc(-c4ccc5c(c4)c4ccccc4n5-c4cc5c(-c6ccc7c(c6)C(c6ccccc6)(c6ccccc6)c6ccccc6-7)nc6ccccc6c5c5c4sc4ccccc45)ccc32)cc1]

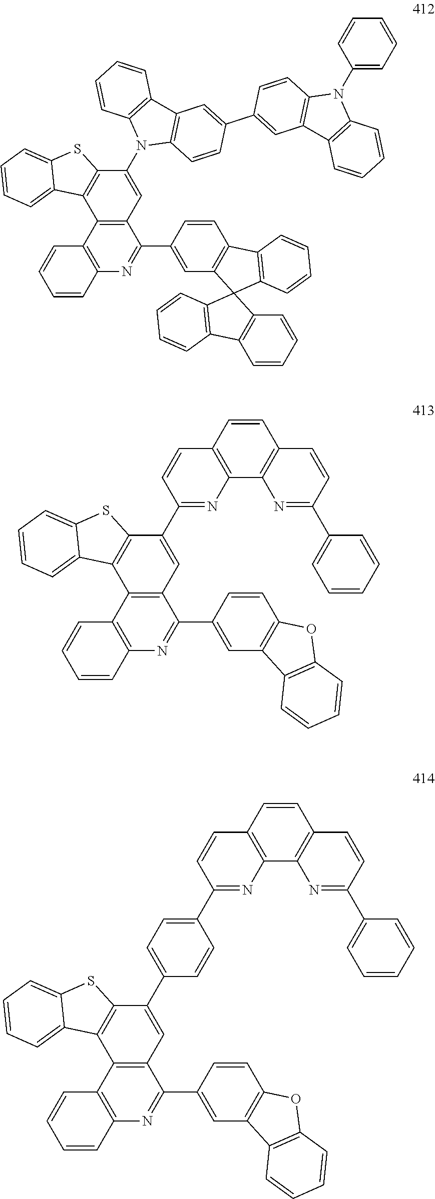 [SMILES: c1ccc(-n2c3ccccc3c3cc(-c4ccc5c(c4)c4ccccc4n5-c4cc5c(-c6ccc7c(c6)C6(c8ccccc8-c8ccccc86)c6ccccc6-7)nc6ccccc6c5c5c4sc4ccccc45)ccc32)cc1 ; c1ccc(-c2ccc3ccc4ccc(-c5cc6c(-c7ccc8oc9ccccc9c8c7)nc7ccccc7c6c6c5sc5ccccc56)nc4c3n2)cc1 ; c1ccc(-c2ccc3ccc4ccc(-c5ccc(-c6cc7c(-c8ccc9oc%10ccccc%10c9c8)nc8ccccc8c7c7c6sc6ccccc67)cc5)nc4c3n2)cc1]

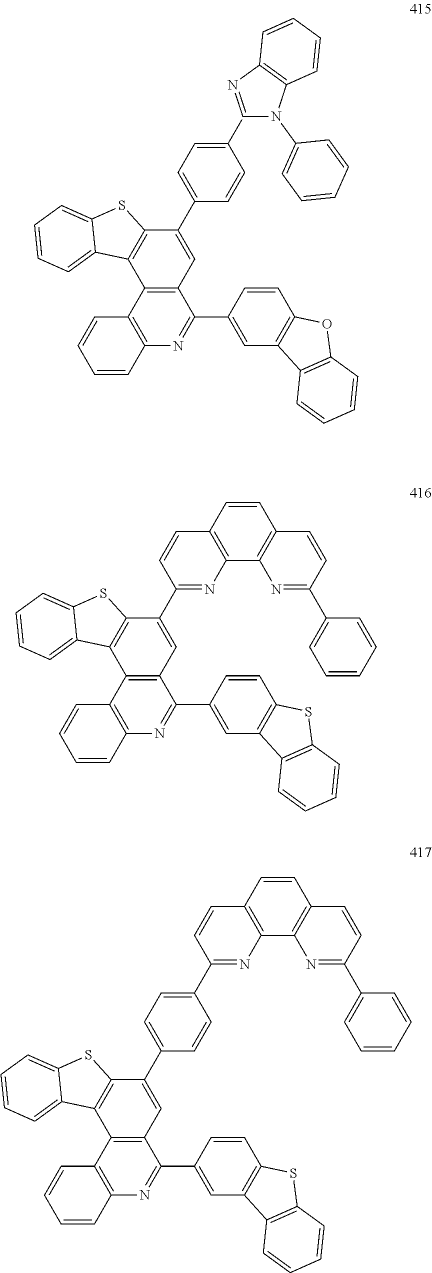

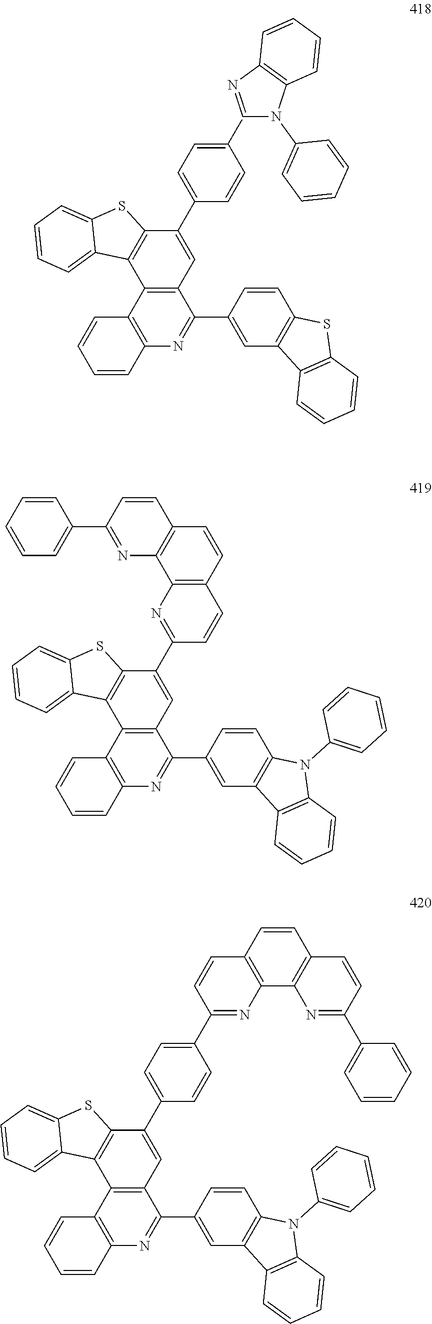

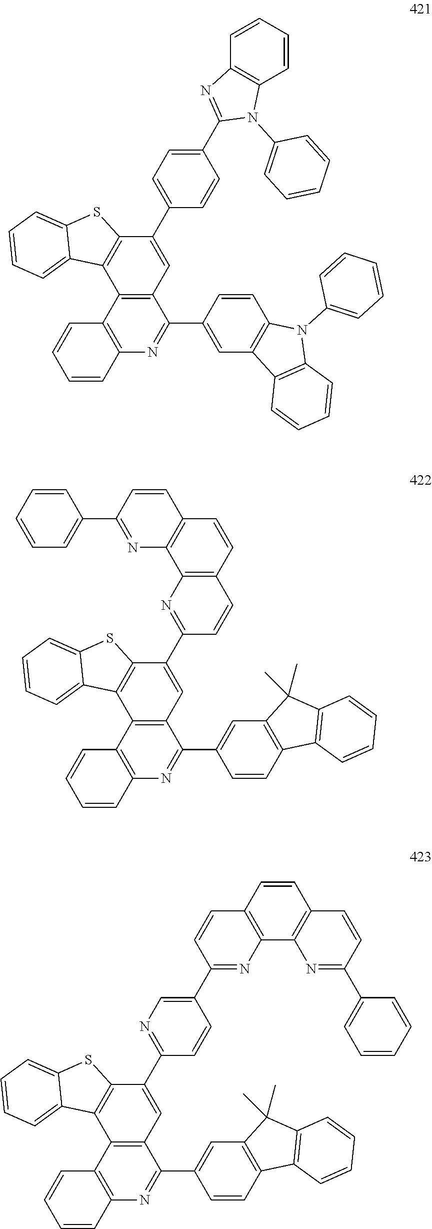 [SMILES: c1ccc(-n2c(-c3ccc(-c4cc5c(-c6ccc7c(c6)c6ccccc6n7-c6ccccc6)nc6ccccc6c5c5c4sc4ccccc45)cc3)nc3ccccc32)cc1 ; CC1(C)c2ccccc2-c2ccc(-c3nc4ccccc4c4c3cc(-c3ccc5ccc6ccc(-c7ccccc7)nc6c5n3)c3sc5ccccc5c34)cc21 ; CC1(C)c2ccccc2-c2ccc(-c3nc4ccccc4c4c3cc(-c3ccc(-c5ccc6ccc7ccc(-c8ccccc8)nc7c6n5)cn3)c3sc5ccccc5c34)cc21]

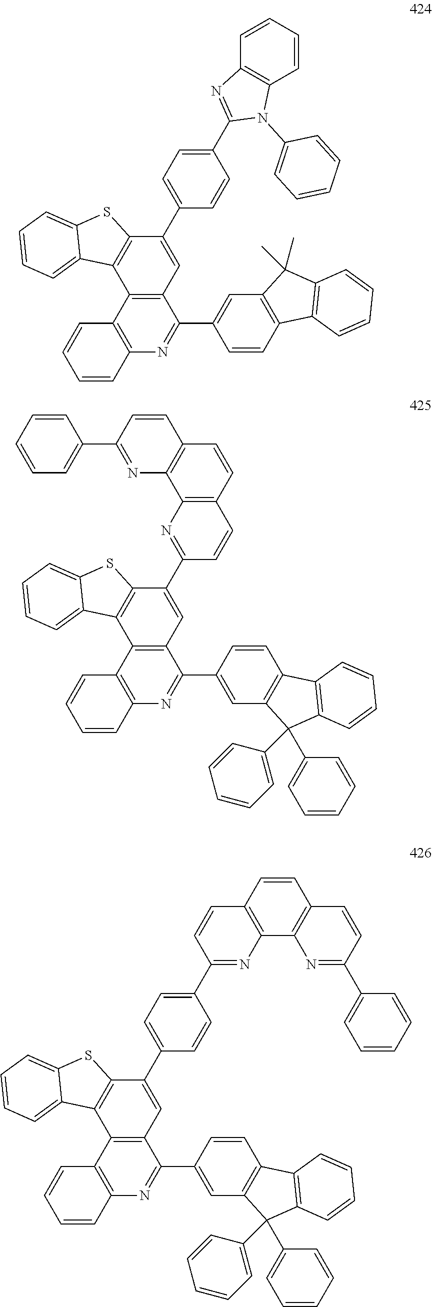

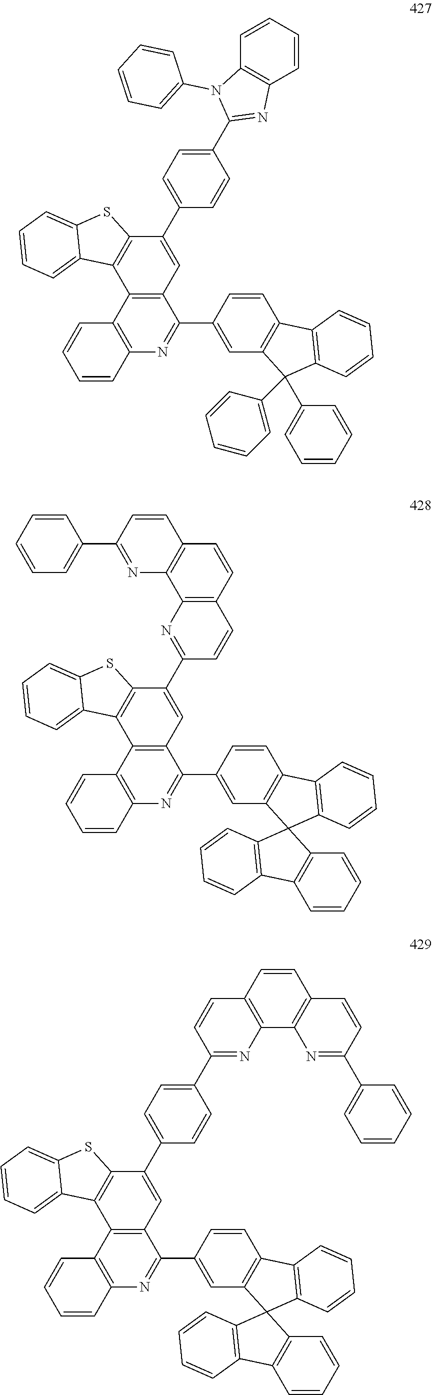 [SMILES: c1ccc(-n2c(-c3ccc(-c4cc5c(-c6ccc7c(c6)C(c6ccccc6)(c6ccccc6)c6ccccc6-7)nc6ccccc6c5c5c4sc4ccccc45)cc3)nc3ccccc32)cc1 ; c1ccc(-c2ccc3ccc4ccc(-c5cc6c(-c7ccc8c(c7)C7(c9ccccc9-c9ccccc97)c7ccccc7-8)nc7ccccc7c6c6c5sc5ccccc56)nc4c3n2)cc1 ; c1ccc(-c2ccc3ccc4ccc(-c5ccc(-c6cc7c(-c8ccc9c(c8)C8(c%10ccccc%10-c%10ccccc%108)c8ccccc8-9)nc8ccccc8c7c7c6sc6ccccc67)cc5)nc4c3n2)cc1]

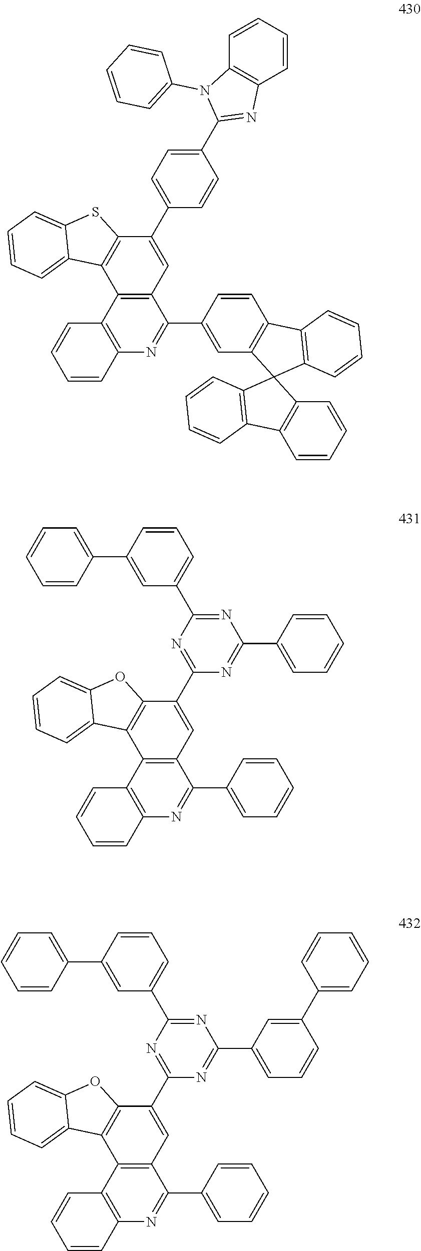 [SMILES: c1ccc(-n2c(-c3ccc(-c4cc5c(-c6ccc7c(c6)C6(c8ccccc8-c8ccccc86)c6ccccc6-7)nc6ccccc6c5c5c4sc4ccccc45)cc3)nc3ccccc32)cc1 ; c1ccc(-c2cccc(-c3nc(-c4ccccc4)nc(-c4cc5c(-c6ccccc6)nc6ccccc6c5c5c4oc4ccccc45)n3)c2)cc1 ; c1ccc(-c2cccc(-c3nc(-c4cccc(-c5ccccc5)c4)nc(-c4cc5c(-c6ccccc6)nc6ccccc6c5c5c4oc4ccccc45)n3)c2)cc1]

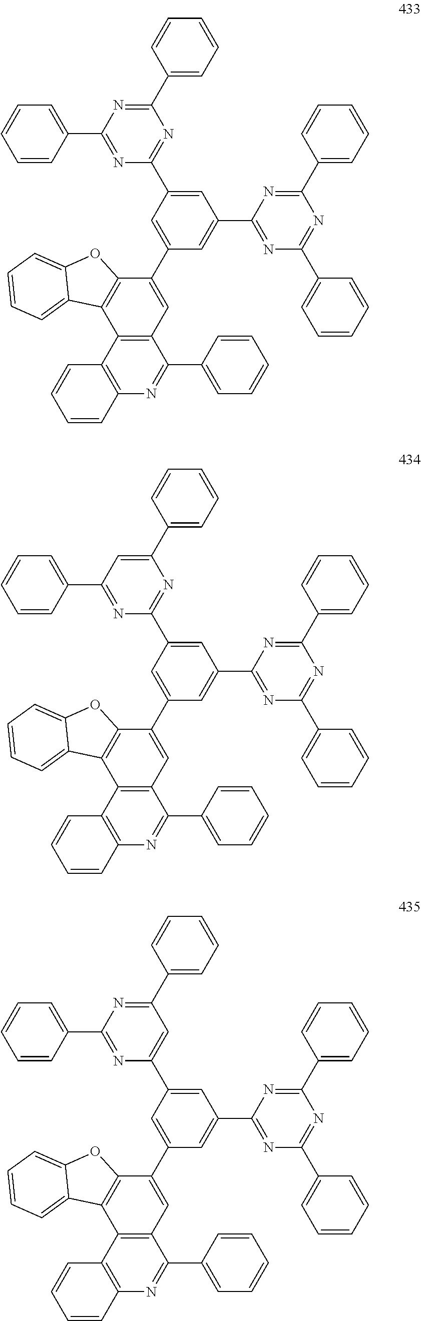 [SMILES: c1ccc(-c2nc(-c3ccccc3)nc(-c3cc(-c4nc(-c5ccccc5)nc(-c5ccccc5)n4)cc(-c4cc5c(-c6ccccc6)nc6ccccc6c5c5c4oc4ccccc45)c3)n2)cc1 ; c1ccc(-c2cc(-c3ccccc3)nc(-c3cc(-c4nc(-c5ccccc5)nc(-c5ccccc5)n4)cc(-c4cc5c(-c6ccccc6)nc6ccccc6c5c5c4oc4ccccc45)c3)n2)cc1 ; c1ccc(-c2cc(-c3cc(-c4nc(-c5ccccc5)nc(-c5ccccc5)n4)cc(-c4cc5c(-c6ccccc6)nc6ccccc6c5c5c4oc4ccccc45)c3)nc(-c3ccccc3)n2)cc1]

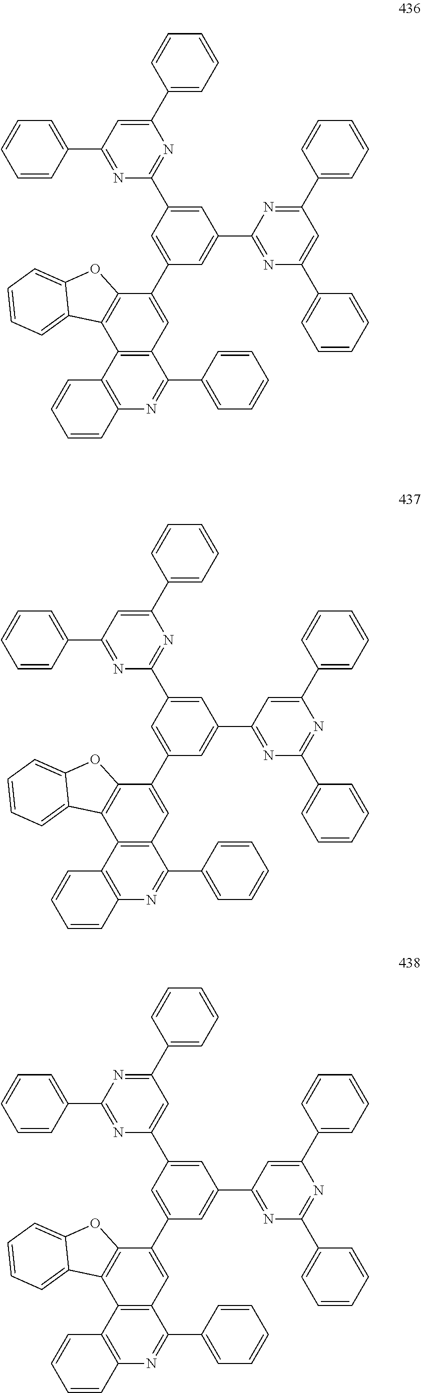 [SMILES: c1ccc(-c2cc(-c3ccccc3)nc(-c3cc(-c4nc(-c5ccccc5)cc(-c5ccccc5)n4)cc(-c4cc5c(-c6ccccc6)nc6ccccc6c5c5c4oc4ccccc45)c3)n2)cc1 ; c1ccc(-c2cc(-c3cc(-c4nc(-c5ccccc5)cc(-c5ccccc5)n4)cc(-c4cc5c(-c6ccccc6)nc6ccccc6c5c5c4oc4ccccc45)c3)nc(-c3ccccc3)n2)cc1 ; c1ccc(-c2cc(-c3cc(-c4cc(-c5ccccc5)nc(-c5ccccc5)n4)cc(-c4cc5c(-c6ccccc6)nc6ccccc6c5c5c4oc4ccccc45)c3)nc(-c3ccccc3)n2)cc1]

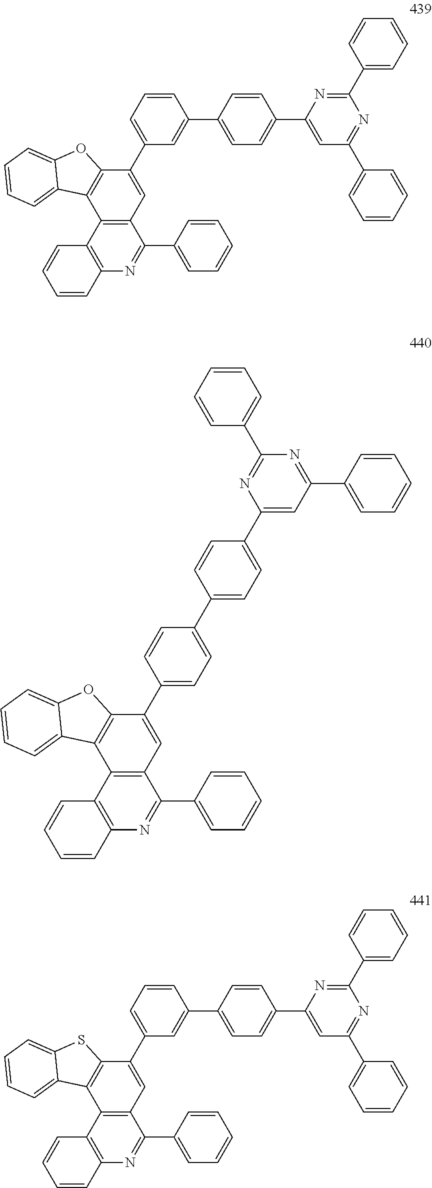 [SMILES: c1ccc(-c2cc(-c3ccc(-c4cccc(-c5cc6c(-c7ccccc7)nc7ccccc7c6c6c5oc5ccccc56)c4)cc3)nc(-c3ccccc3)n2)cc1 ; c1ccc(-c2cc(-c3ccc(-c4ccc(-c5cc6c(-c7ccccc7)nc7ccccc7c6c6c5oc5ccccc56)cc4)cc3)nc(-c3ccccc3)n2)cc1 ; c1ccc(-c2cc(-c3ccc(-c4cccc(-c5cc6c(-c7ccccc7)nc7ccccc7c6c6c5sc5ccccc56)c4)cc3)nc(-c3ccccc3)n2)cc1]

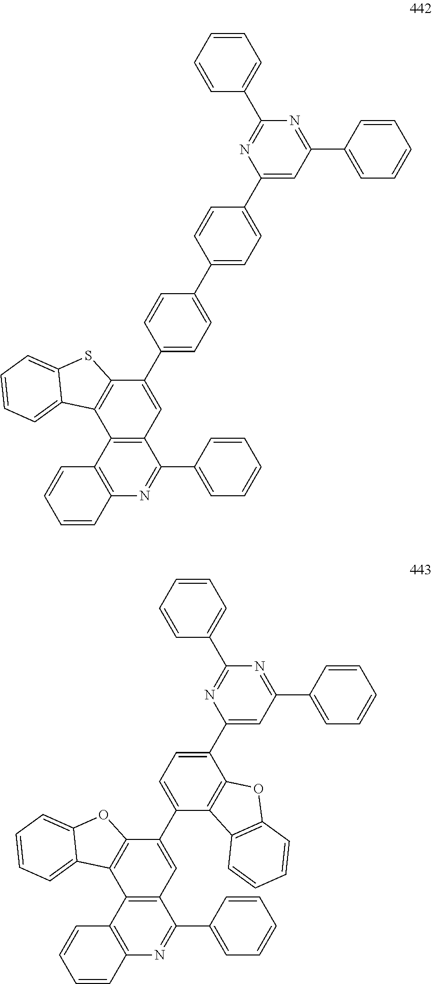 [SMILES: c1ccc(-c2cc(-c3ccc(-c4ccc(-c5cc6c(-c7ccccc7)nc7ccccc7c6c6c5sc5ccccc56)cc4)cc3)nc(-c3ccccc3)n2)cc1 ; c1ccc(-c2cc(-c3ccc(-c4cc5c(-c6ccccc6)nc6ccccc6c5c5c4oc4ccccc45)c4c3oc3ccccc34)nc(-c3ccccc3)n2)cc1]

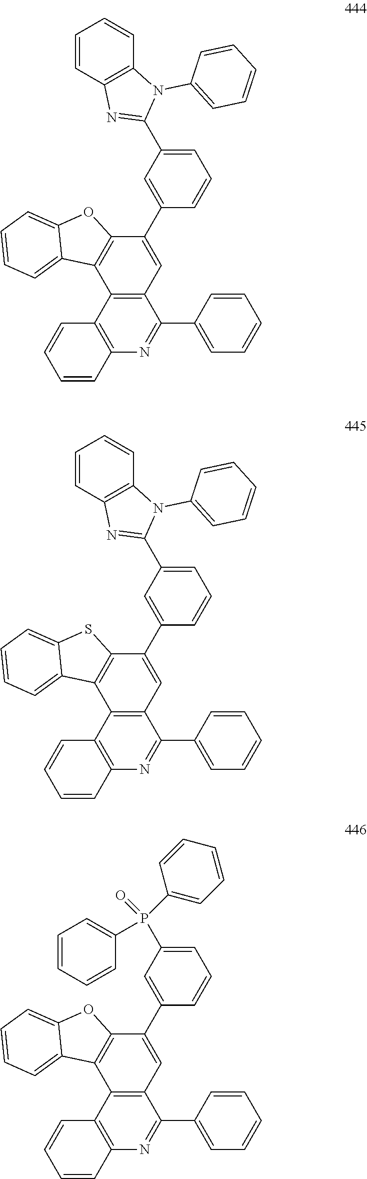 [SMILES: c1ccc(-c2nc3ccccc3c3c2cc(-c2cccc(-c4nc5ccccc5n4-c4ccccc4)c2)c2oc4ccccc4c23)cc1 ; c1ccc(-c2nc3ccccc3c3c2cc(-c2cccc(-c4nc5ccccc5n4-c4ccccc4)c2)c2sc4ccccc4c23)cc1 ; O=P(c1ccccc1)(c1ccccc1)c1cccc(-c2cc3c(-c4ccccc4)nc4ccccc4c3c3c2oc2ccccc23)c1]

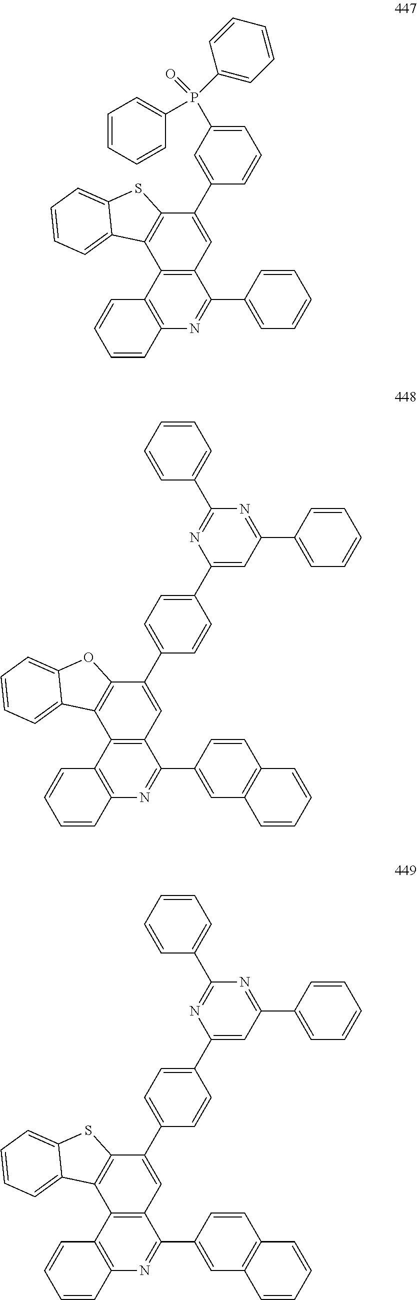 [SMILES: O=P(c1ccccc1)(c1ccccc1)c1cccc(-c2cc3c(-c4ccccc4)nc4ccccc4c3c3c2sc2ccccc23)c1 ; c1ccc(-c2cc(-c3ccc(-c4cc5c(-c6ccc7ccccc7c6)nc6ccccc6c5c5c4oc4ccccc45)cc3)nc(-c3ccccc3)n2)cc1 ; c1ccc(-c2cc(-c3ccc(-c4cc5c(-c6ccc7ccccc7c6)nc6ccccc6c5c5c4sc4ccccc45)cc3)nc(-c3ccccc3)n2)cc1]

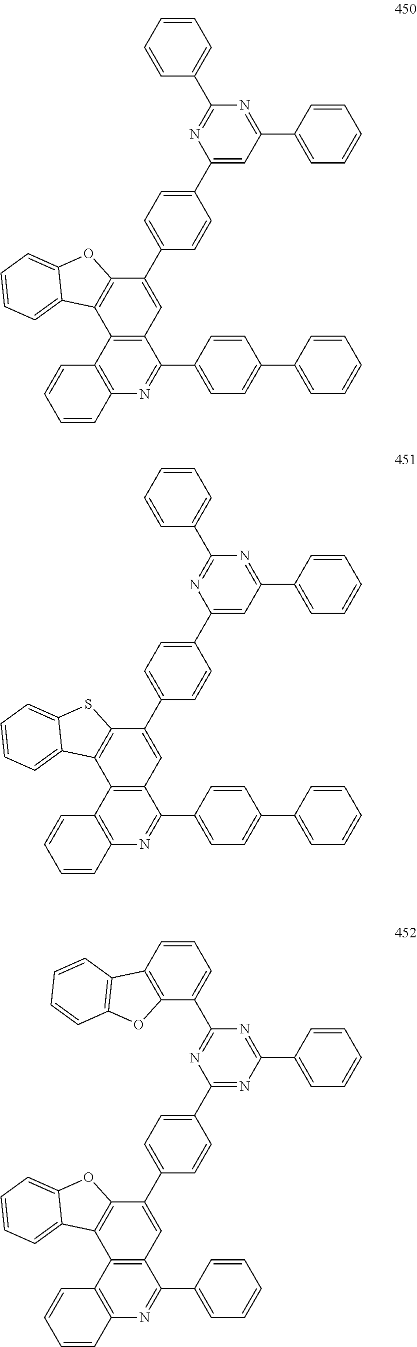 [SMILES: c1ccc(-c2ccc(-c3nc4ccccc4c4c3cc(-c3ccc(-c5cc(-c6ccccc6)nc(-c6ccccc6)n5)cc3)c3oc5ccccc5c34)cc2)cc1 ; c1ccc(-c2ccc(-c3nc4ccccc4c4c3cc(-c3ccc(-c5cc(-c6ccccc6)nc(-c6ccccc6)n5)cc3)c3sc5ccccc5c34)cc2)cc1 ; c1ccc(-c2nc(-c3ccc(-c4cc5c(-c6ccccc6)nc6ccccc6c5c5c4oc4ccccc45)cc3)nc(-c3cccc4c3oc3ccccc34)n2)cc1]

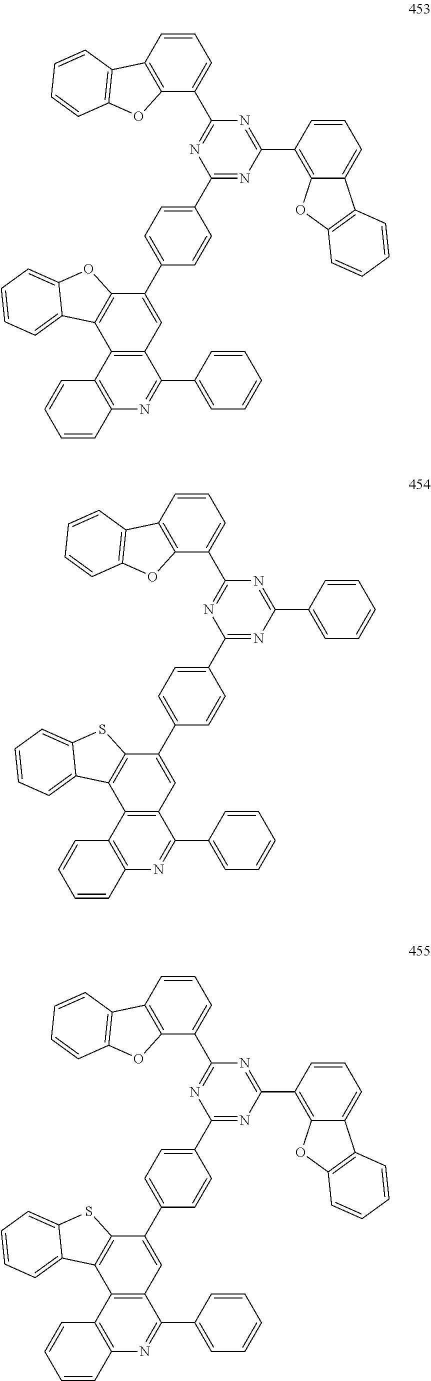 [SMILES: c1ccc(-c2nc3ccccc3c3c2cc(-c2ccc(-c4nc(-c5cccc6c5oc5ccccc56)nc(-c5cccc6c5oc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4c3oc3ccccc34)n2)cc1 ; c1ccc(-c2nc3ccccc3c3c2cc(-c2ccc(-c4nc(-c5cccc6c5oc5ccccc56)nc(-c5cccc6c5oc5ccccc56)n4)cc2)c2sc4ccccc4c23)cc1]

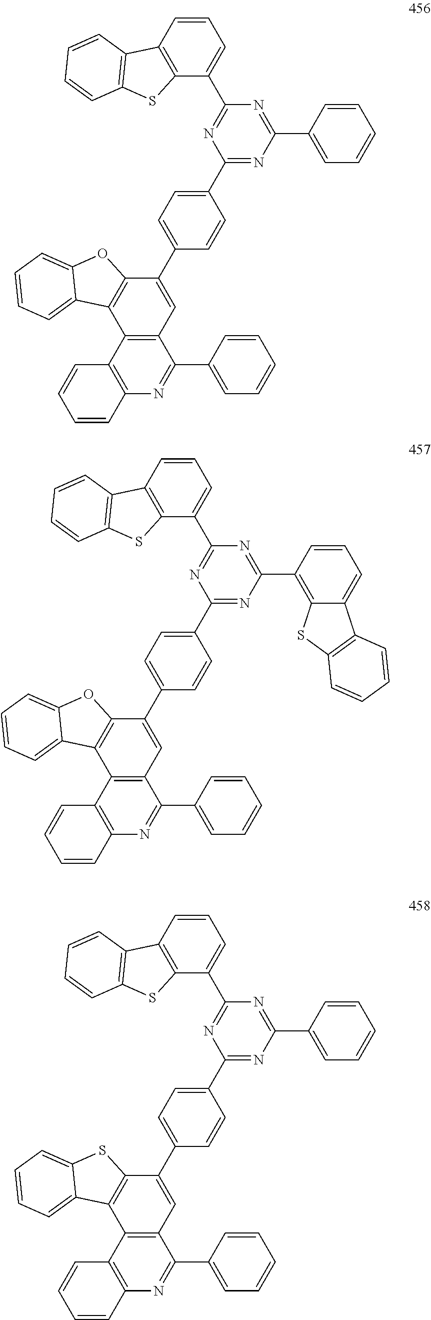 [SMILES: c1ccc(-c2nc(-c3ccc(-c4cc5c(-c6ccccc6)nc6ccccc6c5c5c4oc4ccccc45)cc3)nc(-c3cccc4c3sc3ccccc34)n2)cc1 ; c1ccc(-c2nc3ccccc3c3c2cc(-c2ccc(-c4nc(-c5cccc6c5sc5ccccc56)nc(-c5cccc6c5sc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4c3sc3ccccc34)n2)cc1]

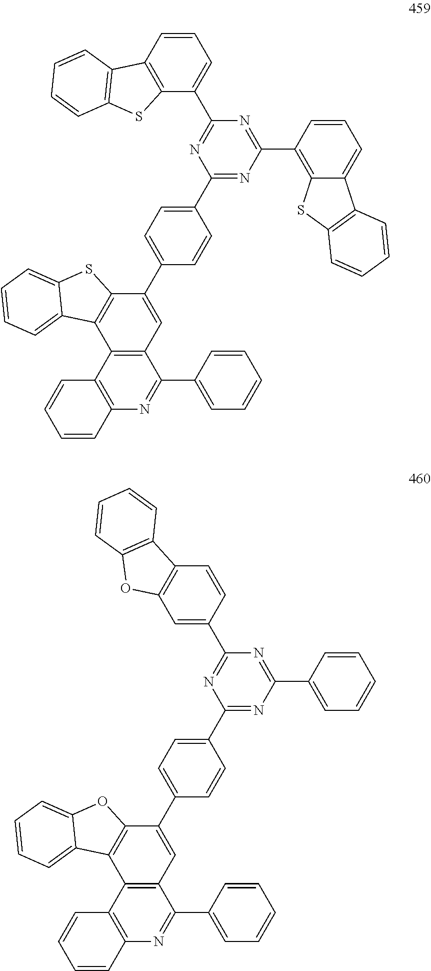 [SMILES: c1ccc(-c2nc3ccccc3c3c2cc(-c2ccc(-c4nc(-c5cccc6c5sc5ccccc56)nc(-c5cccc6c5sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4c(c3)oc3ccccc34)n2)cc1]

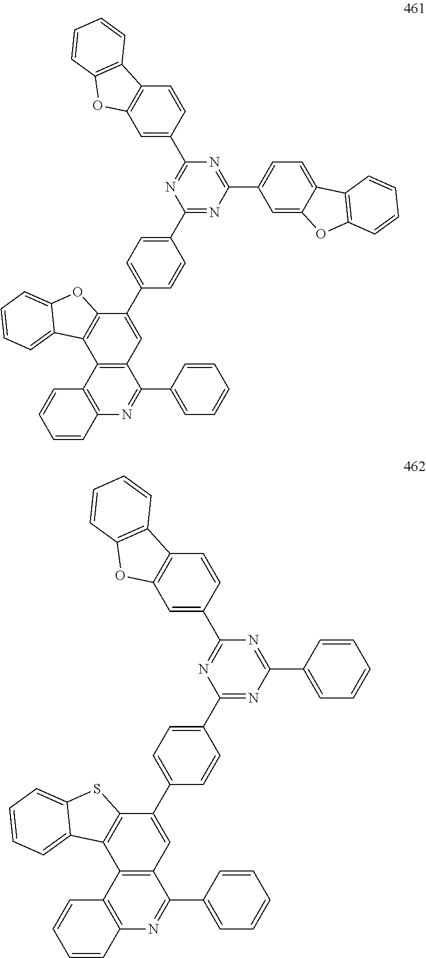 [SMILES: c1ccc(-c2nc3ccccc3c3c2cc(-c2ccc(-c4nc(-c5ccc6c(c5)oc5ccccc56)nc(-c5ccc6c(c5)oc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3ccc4c(c3)oc3ccccc34)n2)cc1]

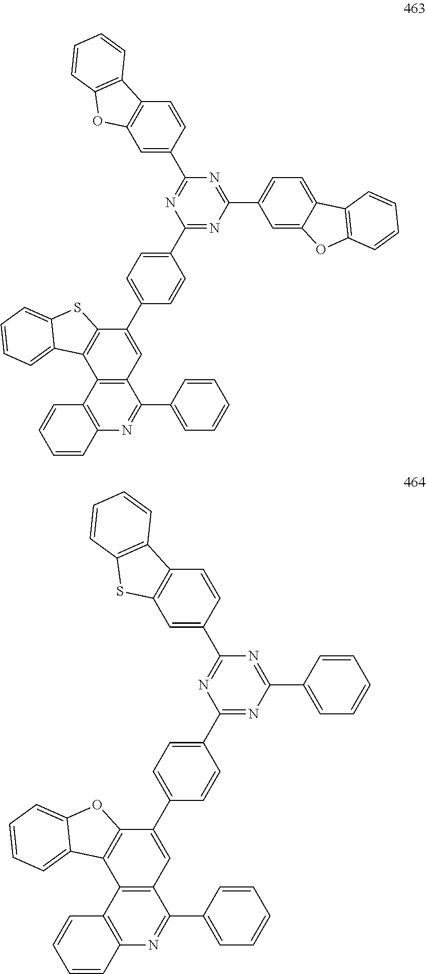 [SMILES: c1ccc(-c2nc3ccccc3c3c2cc(-c2ccc(-c4nc(-c5ccc6c(c5)oc5ccccc56)nc(-c5ccc6c(c5)oc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4c(c3)sc3ccccc34)n2)cc1]

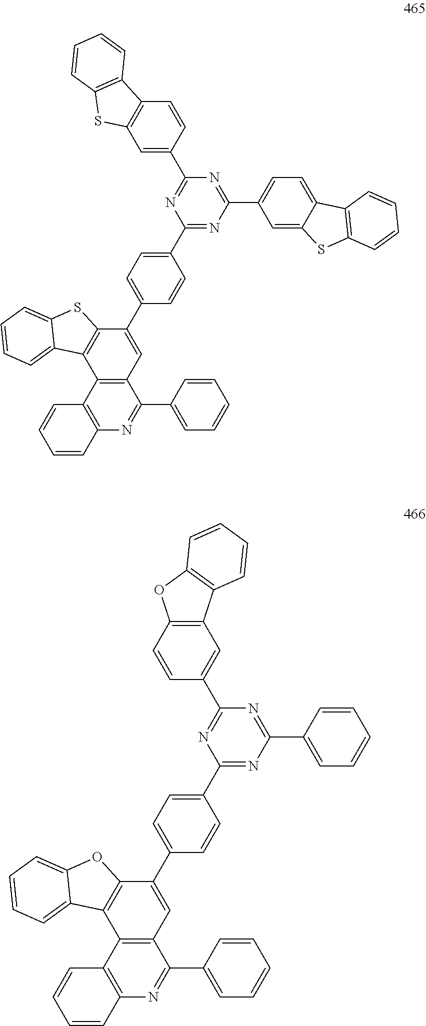 [SMILES: c1ccc(-c2nc3ccccc3c3c2cc(-c2ccc(-c4nc(-c5ccc6c(c5)sc5ccccc56)nc(-c5ccc6c(c5)sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4oc5ccccc5c4c3)n2)cc1]

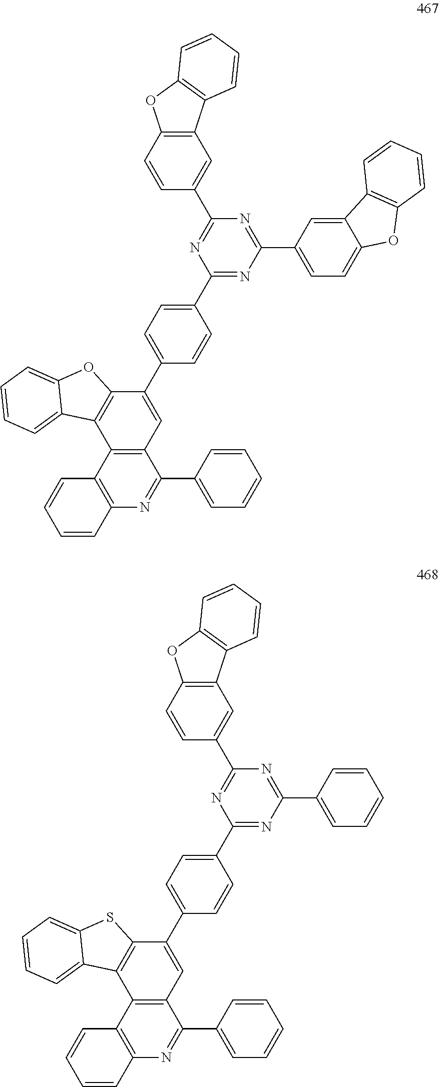 [SMILES: c1ccc(-c2nc3ccccc3c3c2cc(-c2ccc(-c4nc(-c5ccc6oc7ccccc7c6c5)nc(-c5ccc6oc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3ccc4oc5ccccc5c4c3)n2)cc1]

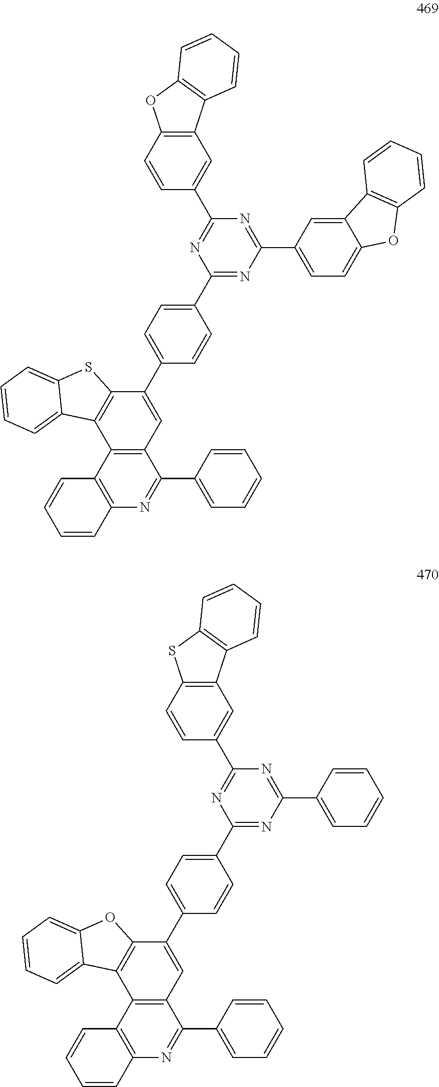 [SMILES: c1ccc(-c2nc3ccccc3c3c2cc(-c2ccc(-c4nc(-c5ccc6oc7ccccc7c6c5)nc(-c5ccc6oc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4sc5ccccc5c4c3)n2)cc1]

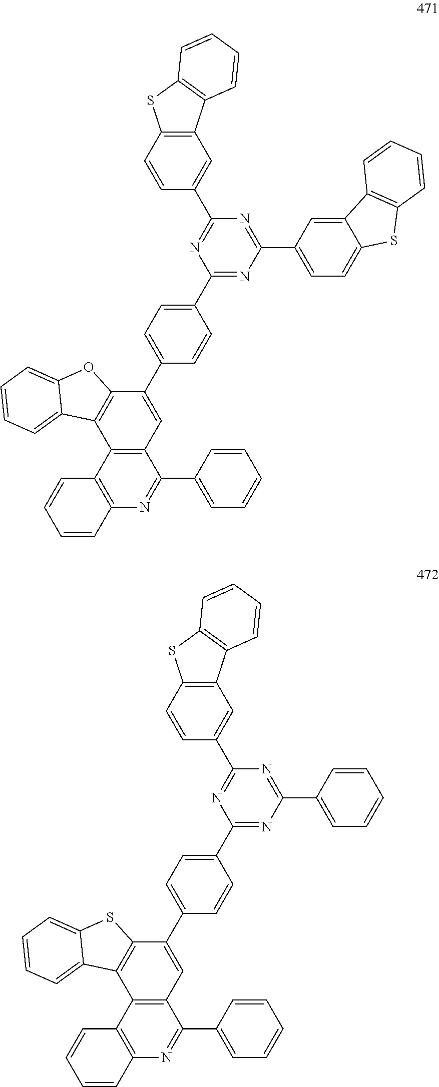 [SMILES: c1ccc(-c2nc3ccccc3c3c2cc(-c2ccc(-c4nc(-c5ccc6sc7ccccc7c6c5)nc(-c5ccc6sc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3ccc4sc5ccccc5c4c3)n2)cc1]

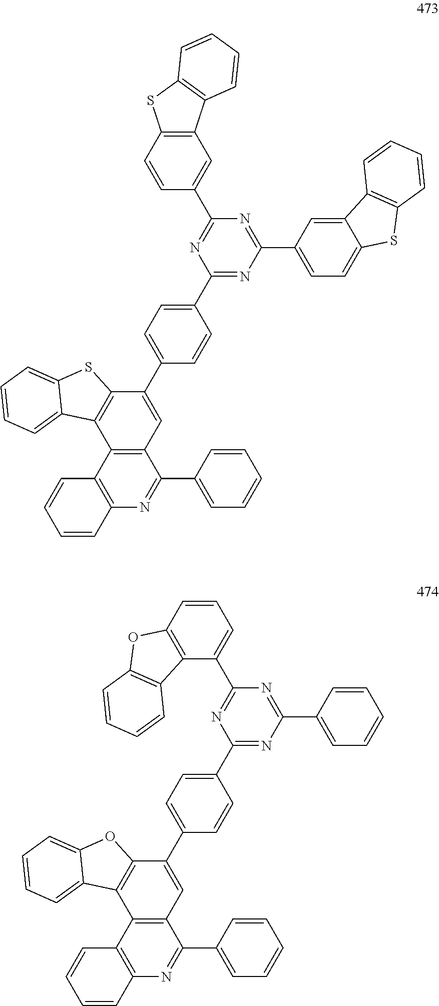 [SMILES: c1ccc(-c2nc3ccccc3c3c2cc(-c2ccc(-c4nc(-c5ccc6sc7ccccc7c6c5)nc(-c5ccc6sc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3cccc4oc5ccccc5c34)n2)cc1]

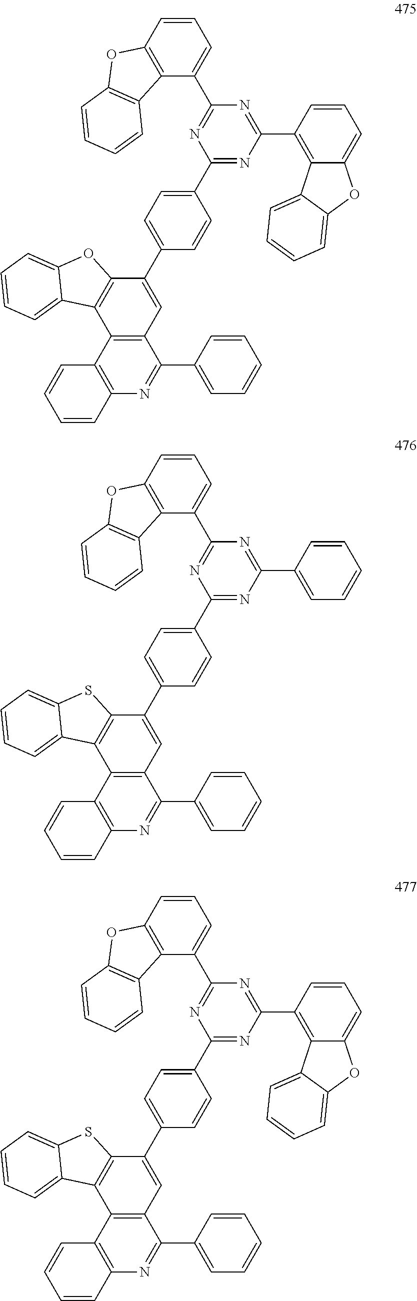 [SMILES: c1ccc(-c2nc3ccccc3c3c2cc(-c2ccc(-c4nc(-c5cccc6oc7ccccc7c56)nc(-c5cccc6oc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4oc5ccccc5c34)n2)cc1 ; c1ccc(-c2nc3ccccc3c3c2cc(-c2ccc(-c4nc(-c5cccc6oc7ccccc7c56)nc(-c5cccc6oc7ccccc7c56)n4)cc2)c2sc4ccccc4c23)cc1]

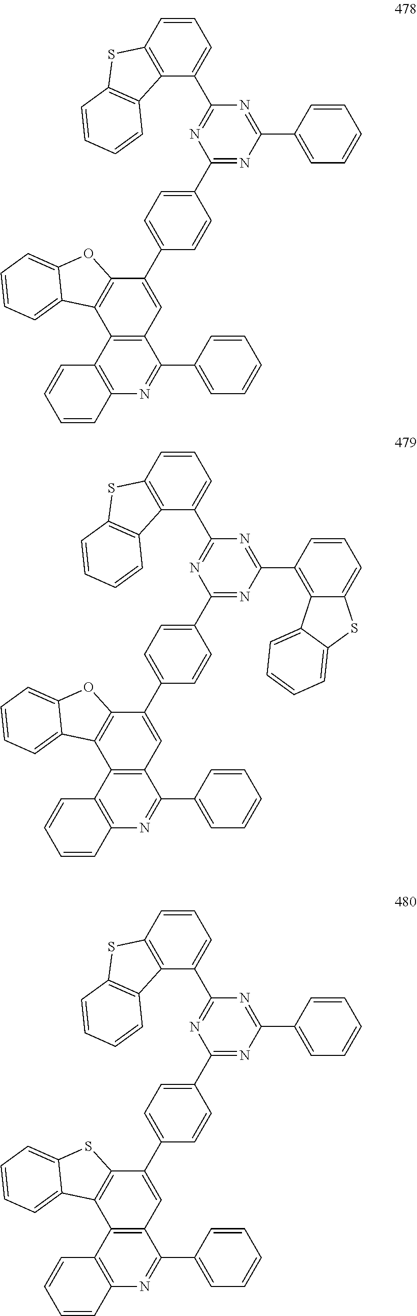 [SMILES: c1ccc(-c2nc(-c3ccc(-c4cc5c(-c6ccccc6)nc6ccccc6c5c5c4oc4ccccc45)cc3)nc(-c3cccc4sc5ccccc5c34)n2)cc1 ; c1ccc(-c2nc3ccccc3c3c2cc(-c2ccc(-c4nc(-c5cccc6sc7ccccc7c56)nc(-c5cccc6sc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4sc5ccccc5c34)n2)cc1]

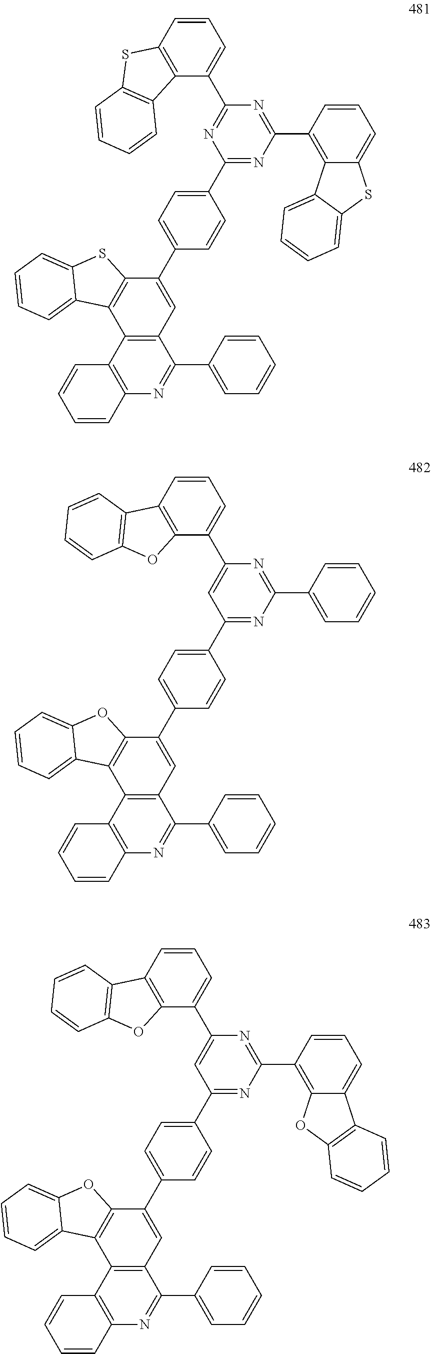 [SMILES: c1ccc(-c2nc3ccccc3c3c2cc(-c2ccc(-c4nc(-c5cccc6sc7ccccc7c56)nc(-c5cccc6sc7ccccc7c56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4c3oc3ccccc34)n2)cc1 ; c1ccc(-c2nc3ccccc3c3c2cc(-c2ccc(-c4cc(-c5cccc6c5oc5ccccc56)nc(-c5cccc6c5oc5ccccc56)n4)cc2)c2oc4ccccc4c23)cc1]

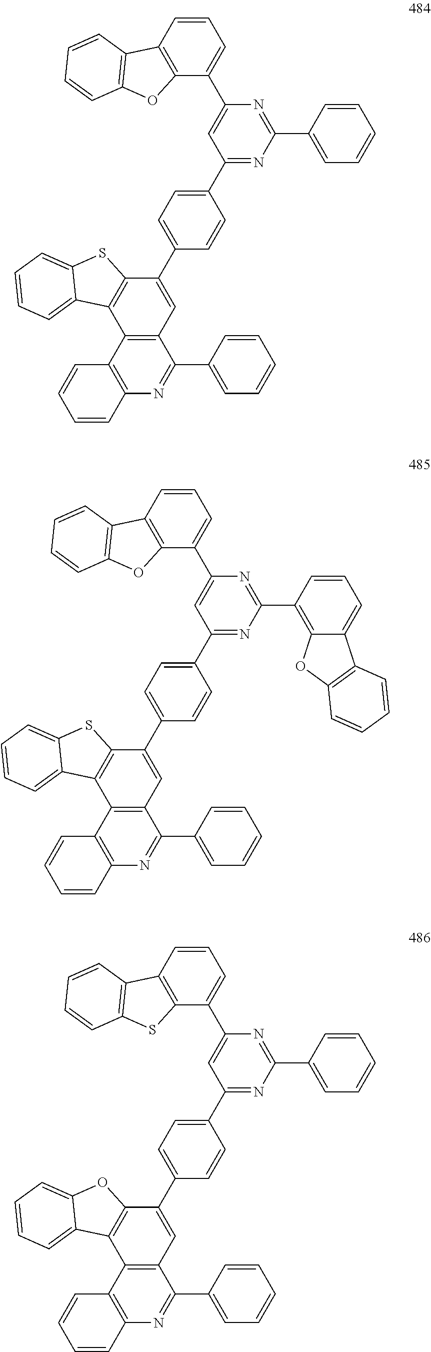 [SMILES: c1ccc(-c2nc(-c3ccc(-c4cc5c(-c6ccccc6)nc6ccccc6c5c5c4sc4ccccc45)cc3)cc(-c3cccc4c3oc3ccccc34)n2)cc1 ; c1ccc(-c2nc3ccccc3c3c2cc(-c2ccc(-c4cc(-c5cccc6c5oc5ccccc56)nc(-c5cccc6c5oc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4c3sc3ccccc34)n2)cc1]

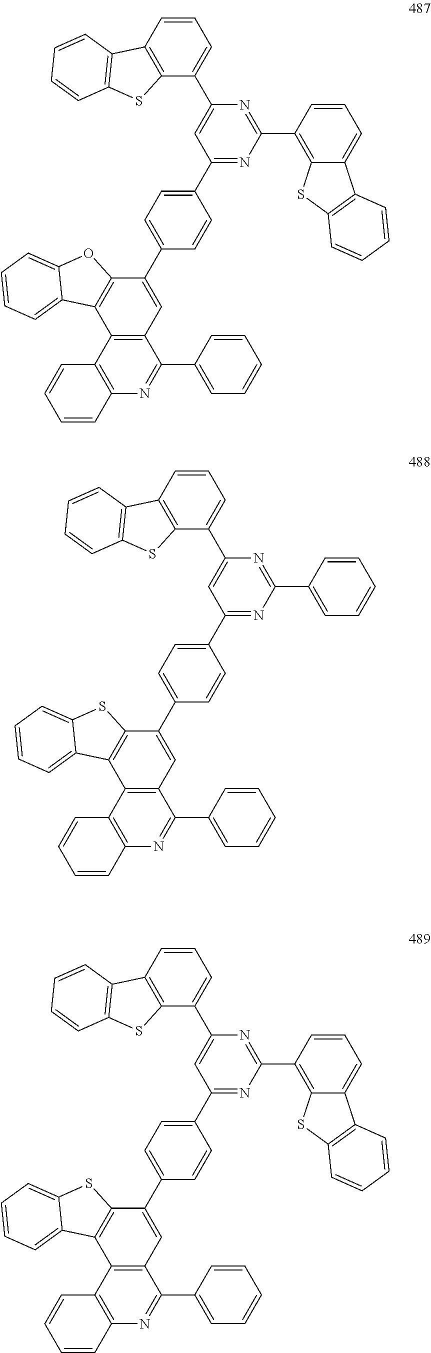 [SMILES: c1ccc(-c2nc3ccccc3c3c2cc(-c2ccc(-c4cc(-c5cccc6c5sc5ccccc56)nc(-c5cccc6c5sc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4c3sc3ccccc34)n2)cc1 ; c1ccc(-c2nc3ccccc3c3c2cc(-c2ccc(-c4cc(-c5cccc6c5sc5ccccc56)nc(-c5cccc6c5sc5ccccc56)n4)cc2)c2sc4ccccc4c23)cc1]

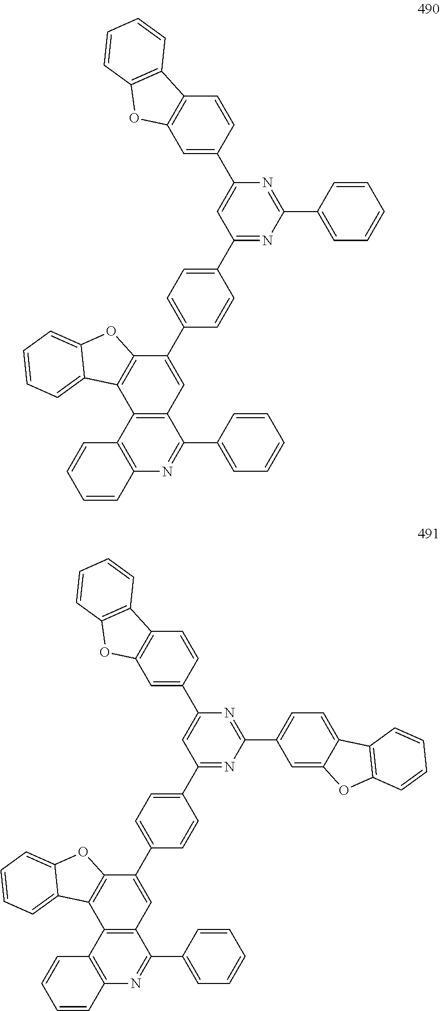 [SMILES: c1ccc(-c2nc(-c3ccc(-c4cc5c(-c6ccccc6)nc6ccccc6c5c5c4oc4ccccc45)cc3)cc(-c3ccc4c(c3)oc3ccccc34)n2)cc1 ; c1ccc(-c2nc3ccccc3c3c2cc(-c2ccc(-c4cc(-c5ccc6c(c5)oc5ccccc56)nc(-c5ccc6c(c5)oc5ccccc56)n4)cc2)c2oc4ccccc4c23)cc1]

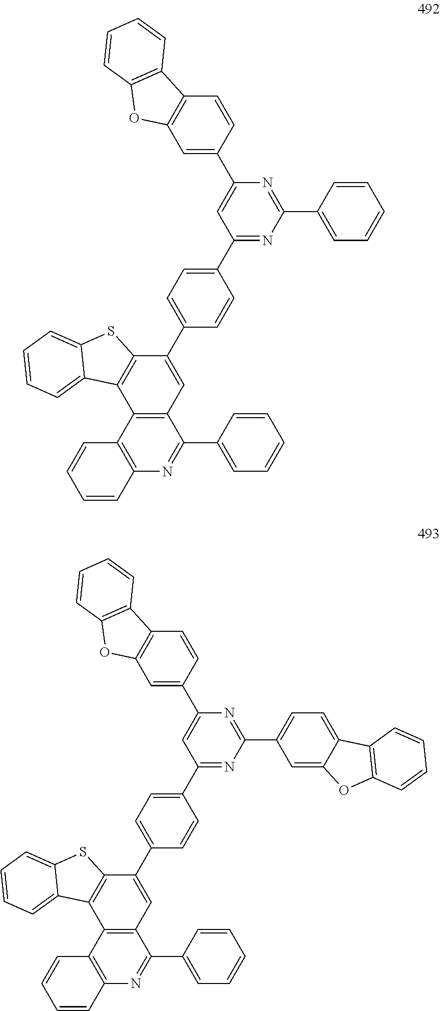 [SMILES: c1ccc(-c2nc(-c3ccc(-c4cc5c(-c6ccccc6)nc6ccccc6c5c5c4sc4ccccc45)cc3)cc(-c3ccc4c(c3)oc3ccccc34)n2)cc1 ; c1ccc(-c2nc3ccccc3c3c2cc(-c2ccc(-c4cc(-c5ccc6c(c5)oc5ccccc56)nc(-c5ccc6c(c5)oc5ccccc56)n4)cc2)c2sc4ccccc4c23)cc1]

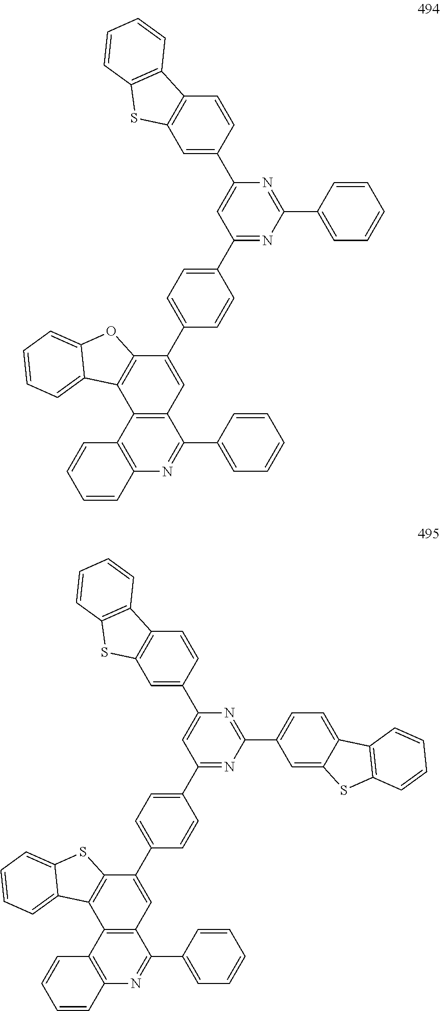 [SMILES: c1ccc(-c2nc(-c3ccc(-c4cc5c(-c6ccccc6)nc6ccccc6c5c5c4oc4ccccc45)cc3)cc(-c3ccc4c(c3)sc3ccccc34)n2)cc1 ; c1ccc(-c2nc3ccccc3c3c2cc(-c2ccc(-c4cc(-c5ccc6c(c5)sc5ccccc56)nc(-c5ccc6c(c5)sc5ccccc56)n4)cc2)c2sc4ccccc4c23)cc1]

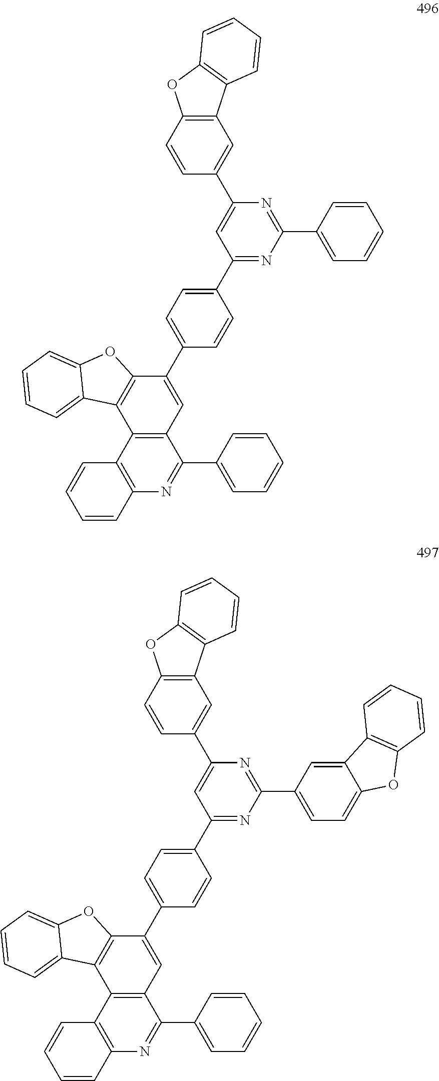 [SMILES: c1ccc(-c2nc(-c3ccc(-c4cc5c(-c6ccccc6)nc6ccccc6c5c5c4oc4ccccc45)cc3)cc(-c3ccc4oc5ccccc5c4c3)n2)cc1 ; c1ccc(-c2nc3ccccc3c3c2cc(-c2ccc(-c4cc(-c5ccc6oc7ccccc7c6c5)nc(-c5ccc6oc7ccccc7c6c5)n4)cc2)c2oc4ccccc4c23)cc1]

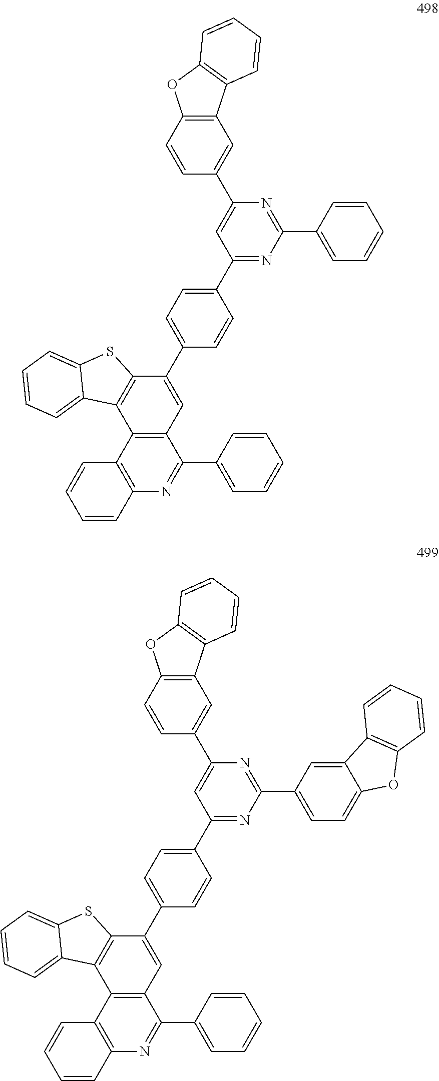 [SMILES: c1ccc(-c2nc(-c3ccc(-c4cc5c(-c6ccccc6)nc6ccccc6c5c5c4sc4ccccc45)cc3)cc(-c3ccc4oc5ccccc5c4c3)n2)cc1 ; c1ccc(-c2nc3ccccc3c3c2cc(-c2ccc(-c4cc(-c5ccc6oc7ccccc7c6c5)nc(-c5ccc6oc7ccccc7c6c5)n4)cc2)c2sc4ccccc4c23)cc1]

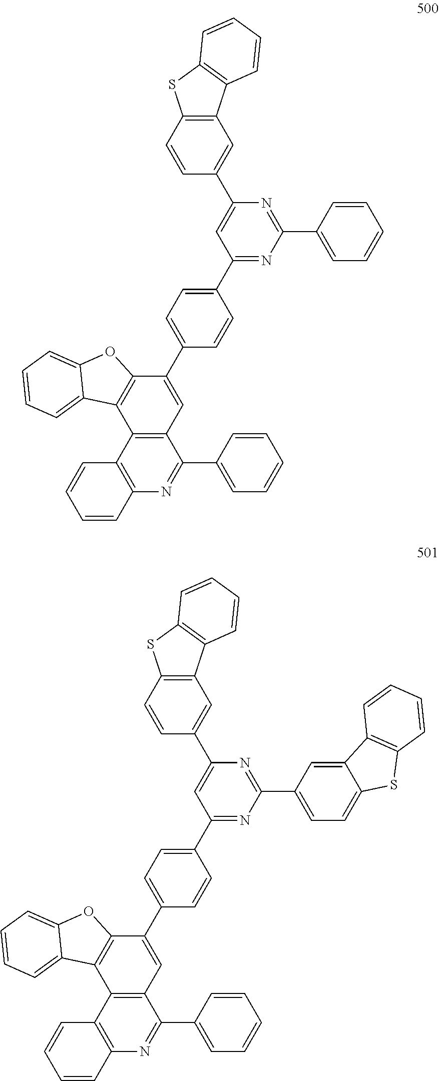 [SMILES: c1ccc(-c2nc(-c3ccc(-c4cc5c(-c6ccccc6)nc6ccccc6c5c5c4oc4ccccc45)cc3)cc(-c3ccc4sc5ccccc5c4c3)n2)cc1 ; c1ccc(-c2nc3ccccc3c3c2cc(-c2ccc(-c4cc(-c5ccc6sc7ccccc7c6c5)nc(-c5ccc6sc7ccccc7c6c5)n4)cc2)c2oc4ccccc4c23)cc1]

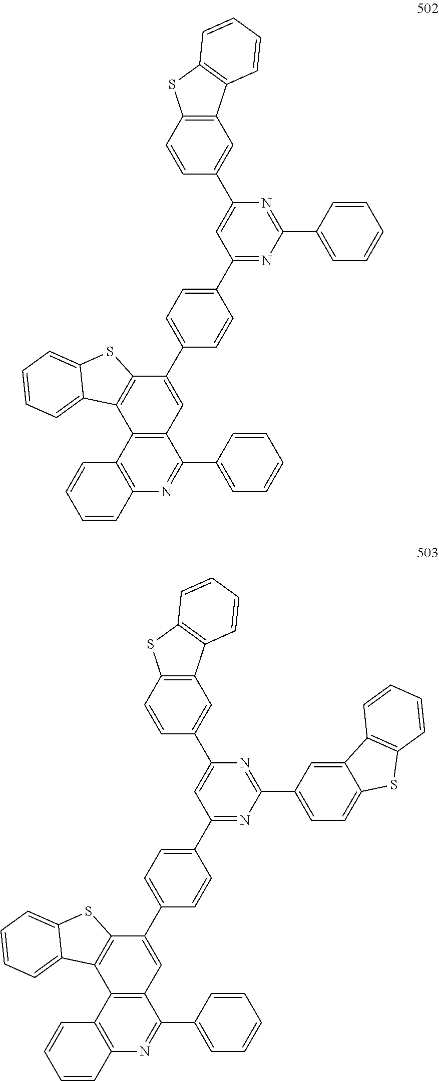 [SMILES: c1ccc(-c2nc(-c3ccc(-c4cc5c(-c6ccccc6)nc6ccccc6c5c5c4sc4ccccc45)cc3)cc(-c3ccc4sc5ccccc5c4c3)n2)cc1 ; c1ccc(-c2nc3ccccc3c3c2cc(-c2ccc(-c4cc(-c5ccc6sc7ccccc7c6c5)nc(-c5ccc6sc7ccccc7c6c5)n4)cc2)c2sc4ccccc4c23)cc1]

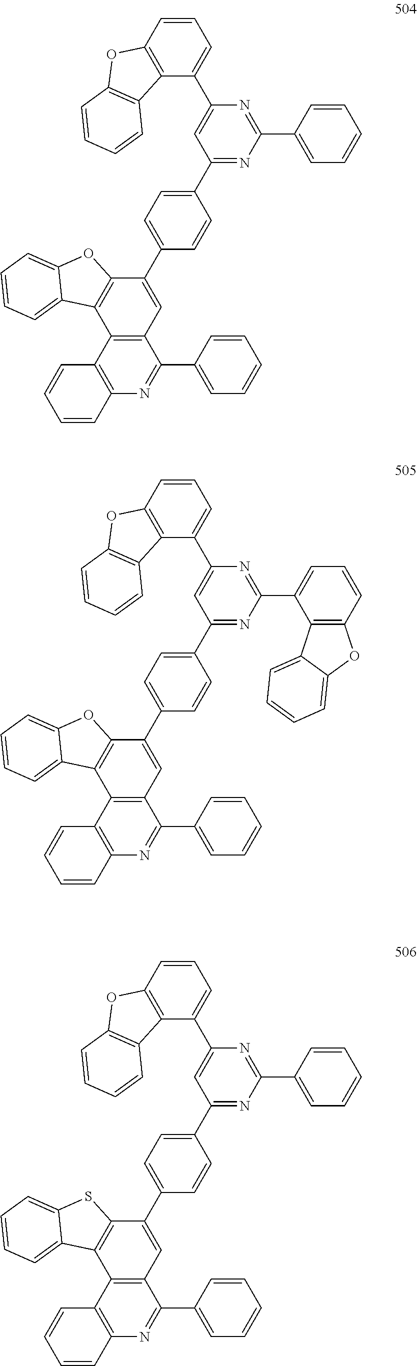 [SMILES: c1ccc(-c2nc(-c3ccc(-c4cc5c(-c6ccccc6)nc6ccccc6c5c5c4oc4ccccc45)cc3)cc(-c3cccc4oc5ccccc5c34)n2)cc1 ; c1ccc(-c2nc3ccccc3c3c2cc(-c2ccc(-c4cc(-c5cccc6oc7ccccc7c56)nc(-c5cccc6oc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4oc5ccccc5c34)n2)cc1]

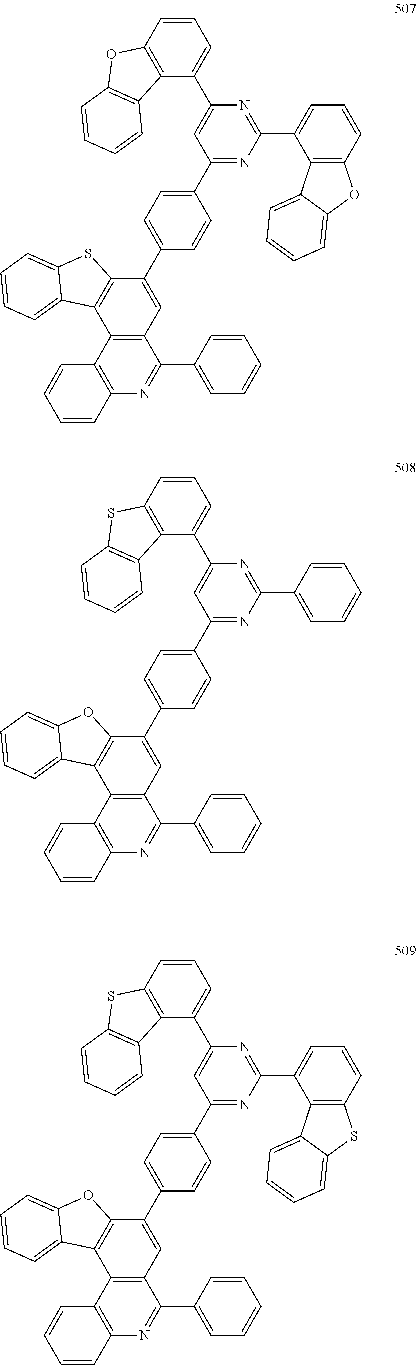 [SMILES: c1ccc(-c2nc3ccccc3c3c2cc(-c2ccc(-c4cc(-c5cccc6oc7ccccc7c56)nc(-c5cccc6oc7ccccc7c56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4sc5ccccc5c34)n2)cc1 ; c1ccc(-c2nc3ccccc3c3c2cc(-c2ccc(-c4cc(-c5cccc6sc7ccccc7c56)nc(-c5cccc6sc7ccccc7c56)n4)cc2)c2oc4ccccc4c23)cc1]

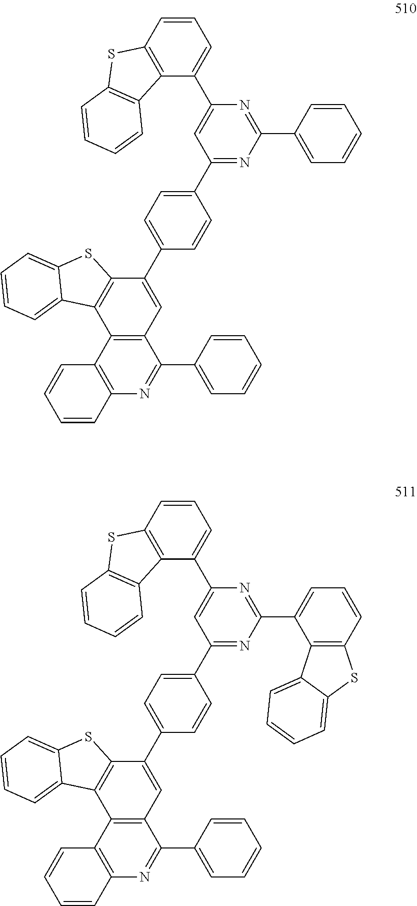 [SMILES: c1ccc(-c2nc(-c3ccc(-c4cc5c(-c6ccccc6)nc6ccccc6c5c5c4sc4ccccc45)cc3)cc(-c3cccc4sc5ccccc5c34)n2)cc1 ; c1ccc(-c2nc3ccccc3c3c2cc(-c2ccc(-c4cc(-c5cccc6sc7ccccc7c56)nc(-c5cccc6sc7ccccc7c56)n4)cc2)c2sc4ccccc4c23)cc1]

In addition, by introducing various substituents into the structure of Chemical Formula 1, it is possible to synthesize a compound having intrinsic characteristics of the introduced substituents. For example, by introducing into the core structure a substituent which is mainly used as a hole injecting layer material, a hole transport layer material, a light emitting layer material, a hole blocking layer material, an electron transport layer material, an electron injecting layer material or a charge generating layer material at the time of manufacturing the organic light emitting device, it is possible to synthesize a material that satisfies the conditions required in each organic material layer.

In addition, by introducing various substituents into the structure of Chemical Formula 1, it is possible to finely control the energy band gap, and further, the characteristics at interfaces between the organic materials can be improved, and the use of the materials can be diversified.

Meanwhile, the compound represented by Chemical Formula 1 has a high glass transition temperature (Tg), allowing it to have excellent thermal stability. This increase in thermal stability is an important factor in providing driving stability to a device.

The compound according to an embodiment of the present invention may be prepared by a multistep chemical reaction. Some intermediate compounds may be prepared first, and the compound represented by Chemical Formula 1 may be prepared from intermediate compounds thereof. More specifically, a method for preparing a compound according to an embodiment of the present invention may be performed as in the following Examples.

Another embodiment of the present invention provides an organic light emitting device comprising the compound represented by Chemical Formula 1.

The organic light emitting device according to an embodiment of the present invention may be manufactured using conventional manufacturing methods and materials of organic light emitting devices, except that one or more of the layered organic material layers are formed using the above-described compound.

The compound represented by Chemical Formula 1 may be formed as an organic material layer by performing a solution coating method as well as a vacuum deposition method at the time of manufacturing the organic light emitting device. Here, the term solution coating method refers to spin coating, dip coating, inkjet printing, screen printing, spraying, roll coating, and the like, but it is not limited thereto. For example, even when the compound represented by Chemical Formula 1 is used as a material for the light emitting layer, the hole blocking layer, the electron transport layer, or the electron injecting layer, an organic material layer can be formed by the solution coating method.

As another example, when the compound represented by Chemical Formula 1 is used to form an organic material layer, the organic material layer below may be formed by the solution coating method, and the organic material layer comprising the compound represented by Chemical Formula 1 may be formed by the vacuum deposition method. Specifically, when the compound represented by Chemical Formula 1 is used as the material for the hole blocking layer, the electron transport layer, or the electron injecting layer, the solution coating method may be used when the light emitting layer is formed on the anode, or when the hole injecting layer and/or the hole transport layer and the light emitting layer are formed on the anode, and the organic material layer comprising the compound represented by Chemical Formula 1 may be formed thereon by using the vacuum deposition method. In this case, even though the organic material layer comprising the compound represented by Chemical Formula 1 is manufactured by the vacuum deposition method, the organic material layer comprising the compound represented by Chemical Formula 1 is well matched with the organic material layer below, formed by the solution coating method.

Specifically, the organic light emitting device according to an embodiment of the present invention comprises an anode, a cathode, and one or more layered organic material layers provided between the anode and the cathode, wherein one or more layers of the organic material layers comprise the compound represented by Chemical Formula 1 above.

That is, the organic material layer comprises at least one of a hole blocking layer, an electron injecting layer, and an electron transporting layer, and at least one layer of the hole blocking layer, the electron injecting layer, and the electron transporting layer comprises the compound represented by Chemical Formula 1 above.

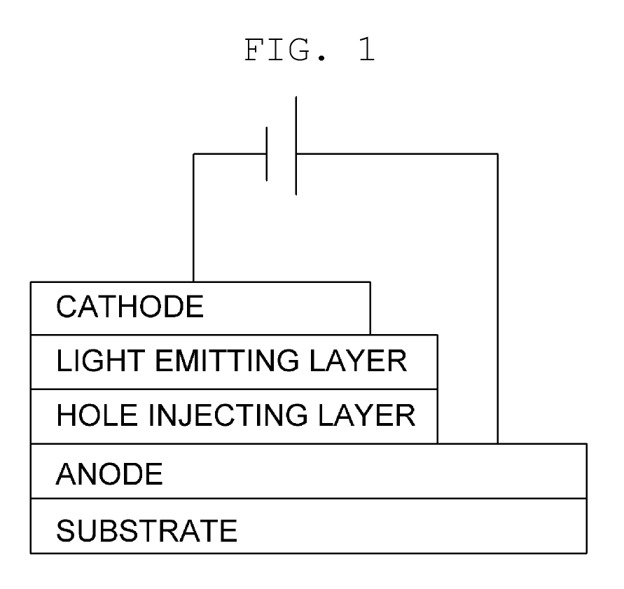

FIG. 1 illustrates a stacking order of an electrode and organic material layers of an organic light emitting device, according to an embodiment of the present invention. However, the above-described drawing is not intended to limit the scope of the present invention, and any structure of an organic light emitting device known in the art may be applied to the present invention.

FIG. 1 illustrates an organic light emitting device in which the anode, the hole injecting layer, the light emitting layer, and the cathode are sequentially stacked on the substrate. However, the present invention is not limited to this structure. Specifically, the compound represented by Chemical Formula 1 may be included in the light emitting layer of the structure illustrated in FIG. 1.

In other words, the organic material layer may comprise a light emitting layer, and the light emitting layer may comprise the compound represented by Chemical Formula 1 above.

Specifically, the organic light emitting device may have a structure of: substrate/anode/light emitting layer/cathode; substrate/anode/hole injecting layer/light emitting layer/cathode; substrate/anode/hole transport layer/light emitting layer/cathode; substrate/anode/hole injecting layer/hole transport layer/light emitting layer/cathode; substrate/anode/hole injecting layer/hole transport layer/light emitting layer/electron transport layer/cathode; substrate/anode/hole injecting layer/hole transport layer/light emitting layer/electron transport layer/electron injecting layer/cathode; substrate/anode/hole injecting layer/hole transport layer/light emitting layer/hole blocking layer/electron transport layer/electron injecting layer/cathode; hole/anode/light emitting layer/electron transport layer/cathode; substrate/anode/light emitting layer/electron injecting layer/cathode; substrate/anode/light emitting layer/hole blocking layer/cathode; substrate/anode/light emitting layer/electron transport layer/electron injecting layer/cathode; substrate/anode/light emitting layer/hole blocking layer/electron transport layer/cathode; substrate/anode/light emitting layer/hole blocking layer/electron transport layer/electron injecting layer/cathode; or the like, wherein one or more of the layered organic material layers between the anode and the cathode, for example, the hole injecting layer, the hole transport layer, the light emitting layer, the hole blocking layer, the electron transport layer or the electron injecting layer, may include the compound represented by Chemical Formula 1. More specifically, the compound represented by Chemical Formula 1 may be used as the material for the light emitting layer, the hole blocking layer, the electron transport layer, or the electron injecting layer in a device having the above structure.

In another embodiment, the organic light emitting device may include a charge generating layer comprising the compound represented by Chemical Formula 1. For example, the organic light emitting device may comprise two or more light emitting units including a light emitting layer, and the charge generating layer may be provided between two adjacent light emitting units. As another example, the organic light emitting device may include one or more light emitting units, and the charge generating layer may be provided between the light emitting unit and the anode, or between the light emitting unit and the cathode.

In other words, the organic material layer may comprise a charge generating layer, and the charge generating layer may comprise the compound represented by Chemical Formula 1 above.

Here, since the charge generating layer comprising the compound represented by Chemical Formula 1 can serve as an n-type charge generating layer, the charge generating layer comprising the compound represented by Chemical Formula 1 may be provided in contact with a p-type organic material layer. Specific examples of the p-type organic material layer are HAT-CN, F₄-TCNQ, a transition metal oxide, and the like.

The light emitting unit may be composed only of a light emitting layer, and it may further include one or more organic material layers such as a hole injecting layer, a hole transport layer, a hole blocking layer, an electron transport layer, an electron injecting layer, and the like, as necessary.

For example, the organic light emitting device may have a structure of: substrate/anode/light emitting unit/charge generating layer (n-type)/charge generating layer (p-type)/light emitting unit/cathode; substrate/anode/charge generating layer (n-type)/charge generating layer (p-type)/light emitting unit/cathode; substrate/anode/light emitting unit/charge generating layer (n-type)/charge generating layer (p-type)/cathode; or the like, wherein the number of light emitting units may be two, three, or more, as necessary. The light emitting unit comprises a light emitting layer, and may further include one or more layers of a hole injecting layer, a hole transport layer, a hole blocking layer, an electron transport layer, and an electron injecting layer, as necessary.

When the compound represented by Chemical Formula 1 is used as a light emitting layer material, the compound represented by Chemical Formula 1 may serve as a light emitting host, and in this case, the light emitting layer further includes a dopant. As an example, the compound represented by Chemical Formula 1 may be employed as a p-type or n-type phosphorescent host, and specifically, may be employed as a phosphorescent green (G) host or a phosphorescent yellow green (YG) host.

In other words, the compound represented by Chemical Formula 1 is a light emitting host, and the light emitting layer may further comprise a light emitting dopant.

As a dopant capable of being used together with the compound represented by Chemical Formula 1, any dopant known in the art may be used. For example, when the compound represented by Chemical Formula 1 is used as a phosphorescent green host, the dopant used together may be Ir(ppy)₃, and the like.

In addition, examples of the dopant used together when the compound represented by Chemical Formula 1 is employed as a phosphorescent YG host may comprise Ir(BT)2(acac), and the like.

The organic light emitting device according to the present invention may be manufactured using materials and methods known in the art, except that the compound represented by Chemical Formula 1 is included in one or more of the organic material layers.

The compound represented by Chemical Formula 1 may be used alone to constitute one or more of the organic material layers of the organic light emitting device. However, if necessary, the organic material layer may be constituted by mixing the compound represented by Chemical Formula 1 with other materials.

In the organic light emitting device according to an embodiment of the present invention, examples of materials other than the compound represented by Chemical Formula 1 which may be used are shown below, but these are provided only by way of example, and are not intended to limit the scope of the present invention, and they may be replaced with other materials known in the art.

As the anode material, materials having a relatively large work function may be used, and a transparent conductive oxide, a metal, a conductive polymer, or the like, may be used.

Specific examples of the anode material include metals such as vanadium, chromium, copper, zinc, gold or their alloys; metal oxides such as zinc oxide, tin oxide, indium tin oxide (ITO), or indium zinc oxide (IZO); combinations of metals and oxides such as Zno:Al or SnO₂:Sb; conductive polymers such as poly(3-methylthiophene), poly[3,4-(ethylene-1,2-dioxy)thiophene](PEDOT), polypyrrole, polyaniline; and the like, but the anode material is not limited thereto.

As the cathode material, materials having a relatively low work function may be used, and a metal, a metal oxide, a conductive polymer, or the like, may be used.

Specific examples of the cathode material include metals such as magnesium, calcium, sodium, potassium, titanium, indium, yttrium, lithium, gadolinium, aluminum, silver, tin, lead, and their alloys; multilayered structured materials such as LiF/Al or Li₀2/Al; and the like, but the cathode material is not limited thereto.

As a hole injecting material, any hole injecting material known in the related art may be used, and it is possible to use, for example, a phthalocyanine compound such as copper phthalocyanine, disclosed in U.S. Pat. No. 4,356,429, starburst-type amine derivatives described in the document [Advanced Material, 6, p. 677 (1994)], for example, tris(4-carbazoyl-9-ylphenyl)amine(TCTA), 4,4′,4″-tri[phenyl(m-tolyl)amino]triphenylamine(m-MTDATA), 1,3,5-tris[4-(3-methylphenyl phenylamino)phenyl]benzene(m-MTDAPB), or the soluble conductive polymer polyaniline/dodecylbenzenesulfonic acid, poly(3,4-ethylenedioxythiophene)/poly(4-styrenesulfonate), polyaniline/camphor sulfonic acid, polyaniline/poly(4-styrene-sulfonate), and the like.

As the hole transport material, a pyrazoline derivative, an arylamine derivative, a stilbene derivative, a triphenyldiamine derivative, or the like, may be used, and a low molecular weight material or a high molecular weight material may be used.

As the electron transport material, an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, may be used, and polymer materials as well as low molecular weight materials may be used.

As the electron injecting material, for example, LiF is generally used in the art, but the present invention is not limited thereto.

As the light emitting material, red, green or blue light emitting material may be used, and if necessary, two or more light emitting materials may be mixed to be used. Further, the light emitting material may be a fluorescent material, but may also be a phosphorescent material. As the light emitting material, a material that emits light by coupling holes and electrons injected from the anode and cathode, respectively, may be used alone, but materials in which both the host material and the dopant material are involved in light emitting may also be used.

The organic light emitting device according to an embodiment of the present invention may be a front emission type, a back emission type, or a double-sided emission type, depending on the material used.

The heterocyclic compound according to an embodiment of the present invention may act on a principle similar to a case applied to organic light emitting devices used among organic electronic devices, including organic solar cells, organic photoconductors, organic transistors, and the like.

Hereinafter, although the present disclosure has been described in detail with reference to Examples, it should be understood that these Examples are provided for illustrative purposes and do not limit the scope of the present disclosure.

### EXAMPLES

### Preparation Example

<Preparation of Core 1-1>

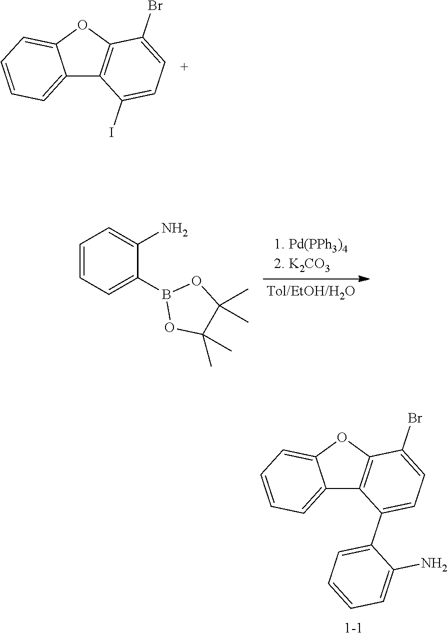 [SMILES: Brc1ccc(I)c2c1oc1ccccc12 ; CC1(C)OB(c2ccccc2N)OC1(C)C ; Nc1ccccc1-c1ccc(Br)c2oc3ccccc3c12]

A 2 L round-bottom flask was charged with 55.1 g (147.72 mmol, 1 eq.) of 1-bromo-4-iododibenzofurane, 32.4 g (147.72 mmol, 1 eq.) of 2-amino-phenylpinacolborane, 8.5 g (7.39 mmol, 0.05 eq.) of Pd(PPh₃)₄, and 61.3 g (443.18 mmol, 3 eq.) of K₂CO₃, then toluene/EtOH/H₂O (500 ml/100 ml/100 ml) was added thereto, and the mixture was stirred under reflux.

After completion of the reaction, the mixture was extracted with MC/H₂O and the MC layer was dried over MgSO₄. Purification was performed using a silica-gel column to obtain 28.6 g of Compound 1-1 at a yield of 57%.

<Preparation of Core 1-2>

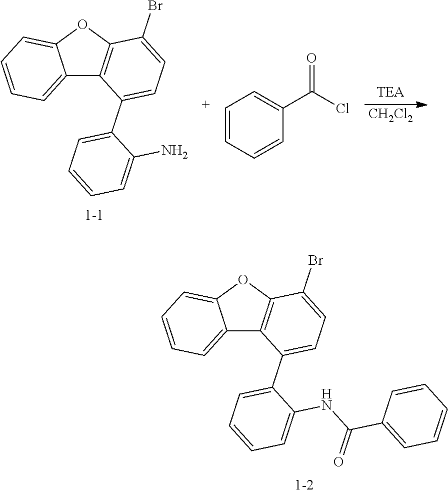

A 2 L round-bottom flask was charged with 28.6 g (84.57 mmol, 1 eq.) of Core 1-1 and 14.2 ml (101.48 mmol, 1.2 eq.) of triethanolamine (TEA), and the mixture was dissolved in 800 ml of dichloromethane (CH₂Cl₂), followed by cooling to 0° C. To the cooled mixture, 10.8 ml (93.02 mmol, 1.1 eq.) of benzoylchloride was added dropwise, and the mixture was heated to room temperature and stirred.

After completion of the reaction, the mixture was extracted with MC/H₂O and the MC layer was dried over MgSO₄. Purification was performed using a silica-gel column to obtain 35 g of Core 1-2 at a yield of 94%.

<Preparation of Core 1-3>

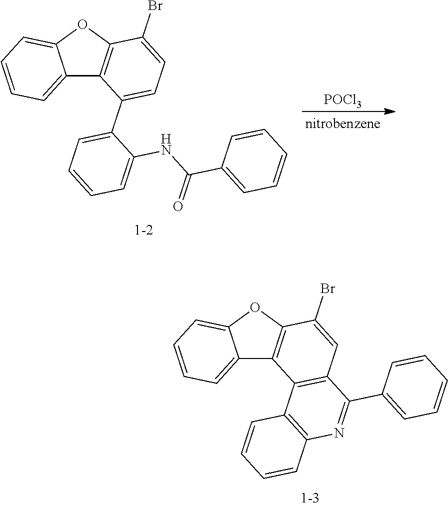 [SMILES: O=C(Nc1ccccc1-c1ccc(Br)c2oc3ccccc3c12)c1ccccc1 ; "POCl3" ; Brc1cc2c(-c3ccccc3)nc3ccccc3c2c2c1oc1ccccc12]

A 1 L round-bottom flask was charged with 28.5 g (64.43 mmol, 1 eq.) of Core 1-2 and 4.2 ml (45.10 mmol, 0.7 eq.) of POCl₃, then 600 ml of nitrobenzene was added thereto, and the mixture was stirred at 150° C.

After completion of the reaction, the mixture was extracted with MC/H₂O and the MC layer was dried over MgSO₄. Purification was performed using a silica-gel column to obtain 26.5 g of Core 1-3 at a yield of 97%.

<Preparation of Core 1-4>

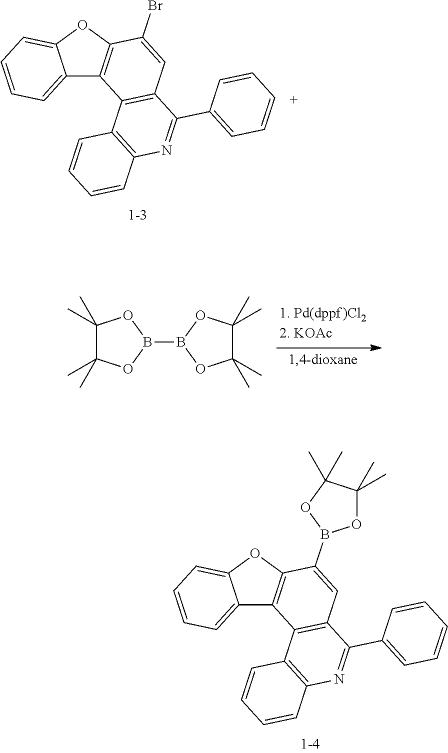

A 500 ml round-bottom flask was charged with 17.5 g (41.24 mmol, 1 eq.) of Core 1-3, 15.7 g (61.87 mmol, 1.5 eq.) of [bis(pinacolato)diboron], 1.5 g (2.06 mmol, 0.05 eq.) of Pd(dppf)Cl₂, and 16.2 g (164.98 mmol, 4 eq.) of potassium acetate (KOAc), then 300 ml of 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 14 g of Core 1-4 at a yield of 72%.

<General Procedure A>

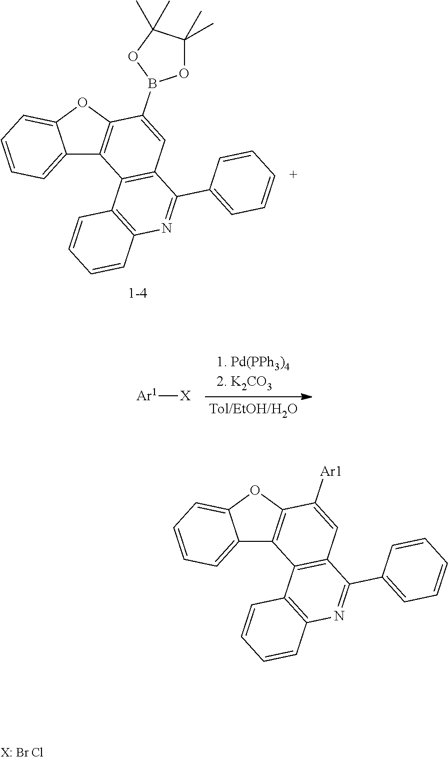

A 500 ml round-bottom flask was charged with Core 1-4 (1.1 eq.), Ar1-X (1 eq.) described in Table 1 below, Pd(PPh₃)₄ (0.05 eq.) and K₂CO₃ (3 eq.), then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 1

| Com- | | Yield | |
|---|---|---|---|
| pound | Ar1—X | (%) | Structure |
| 19 | 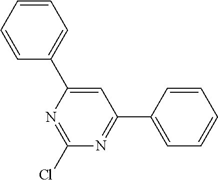 | 83 | 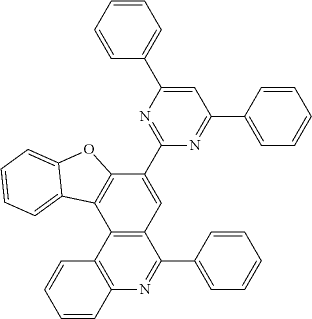 [SMILES: c1ccc(-c2cc(-c3ccccc3)nc(-c3cc4c(-c5ccccc5)nc5ccccc5c4c4c3oc3ccccc34)n2)cc1] |
| 23 | 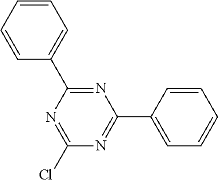 | 89 | 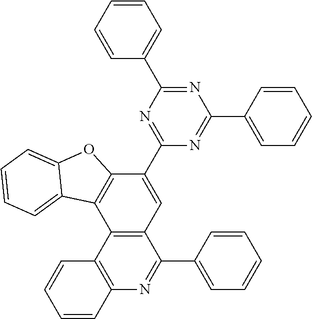 [SMILES: c1ccc(-c2nc(-c3ccccc3)nc(-c3cc4c(-c5ccccc5)nc5ccccc5c4c4c3oc3ccccc34)n2)cc1] |
| 25 | 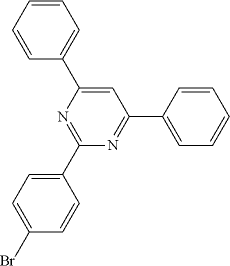 | 81 | 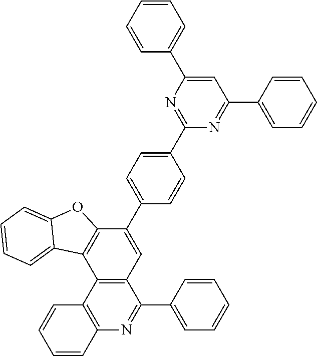 [SMILES: c1ccc(-c2cc(-c3ccccc3)nc(-c3ccc(-c4cc5c(-c6ccccc6)nc6ccccc6c5c5c4oc4ccccc45)cc3)n2)cc1] |
| 26 | 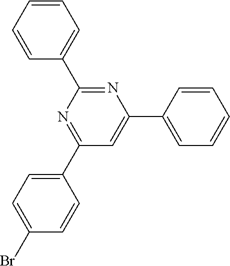 | 86 | 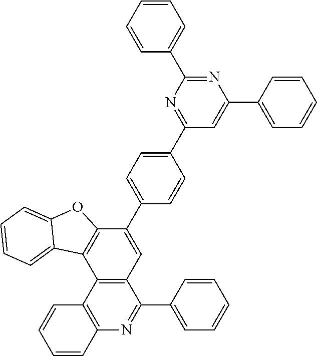 [SMILES: c1ccc(-c2cc(-c3ccc(-c4cc5c(-c6ccccc6)nc6ccccc6c5c5c4oc4ccccc45)cc3)nc(-c3ccccc3)n2)cc1] |
| 28 | 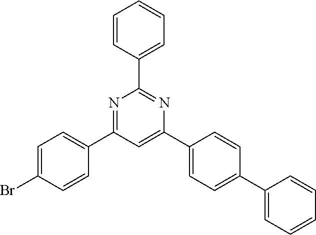 | 79 | 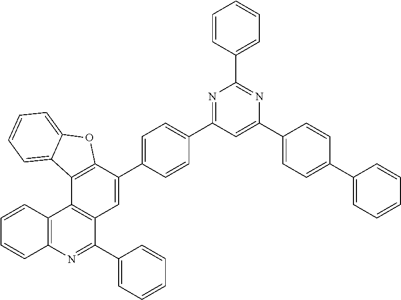 [SMILES: c1ccc(-c2ccc(-c3cc(-c4ccc(-c5cc6c(-c7ccccc7)nc7ccccc7c6c6c5oc5ccccc56)cc4)nc(-c4ccccc4)n3)cc2)cc1] |
| 29 | 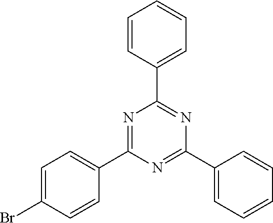 | 91 | 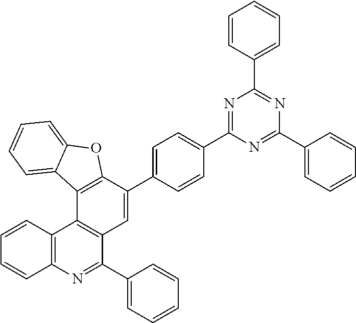 [SMILES: c1ccc(-c2nc(-c3ccccc3)nc(-c3ccc(-c4cc5c(-c6ccccc6)nc6ccccc6c5c5c4oc4ccccc45)cc3)n2)cc1] |
| 31 | 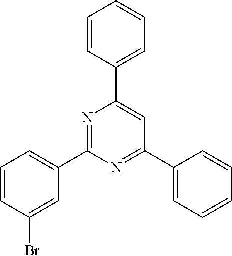 | 76 | 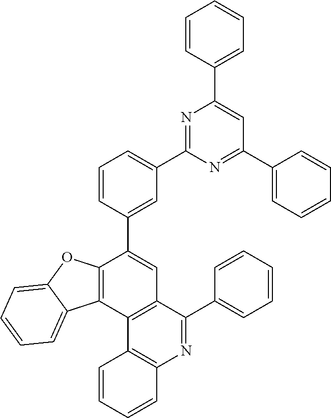 [SMILES: c1ccc(-c2cc(-c3ccccc3)nc(-c3cccc(-c4cc5c(-c6ccccc6)nc6ccccc6c5c5c4oc4ccccc45)c3)n2)cc1] |
| 35 | 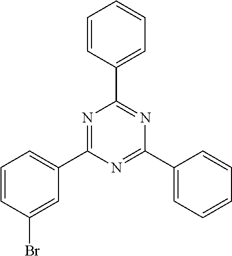 | 89 | 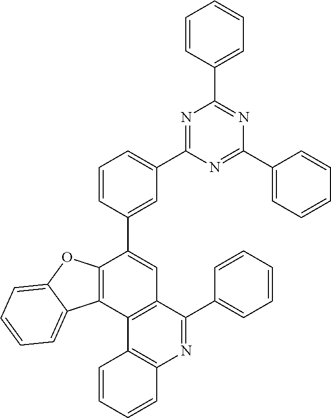 [SMILES: c1ccc(-c2nc(-c3ccccc3)nc(-c3cccc(-c4cc5c(-c6ccccc6)nc6ccccc6c5c5c4oc4ccccc45)c3)n2)cc1] |
| 41 | 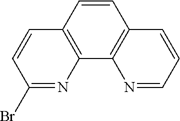 | 94 | 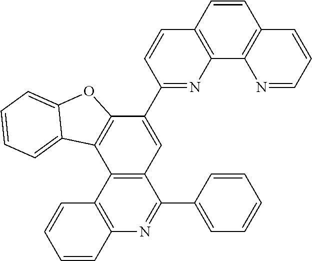 [SMILES: c1ccc(-c2nc3ccccc3c3c2cc(-c2ccc4ccc5cccnc5c4n2)c2oc4ccccc4c23)cc1] |
| 43 | 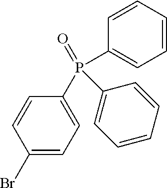 | 91 | 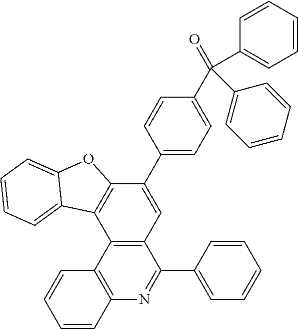 |
| 44 | 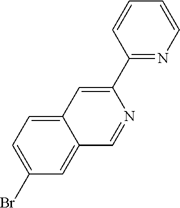 | 81 | 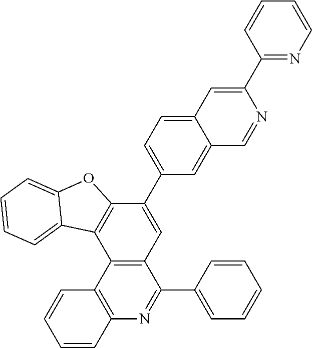 [SMILES: c1ccc(-c2nc3ccccc3c3c2cc(-c2ccc4cc(-c5ccccn5)ncc4c2)c2oc4ccccc4c23)cc1] |
| 45 | 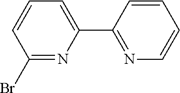 | 71 | 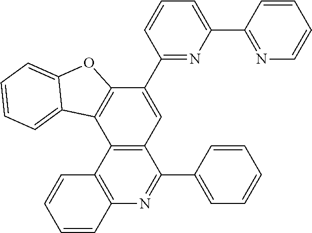 [SMILES: c1ccc(-c2nc3ccccc3c3c2cc(-c2cccc(-c4ccccn4)n2)c2oc4ccccc4c23)cc1] |
| 48 | 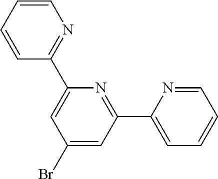 | 68 | 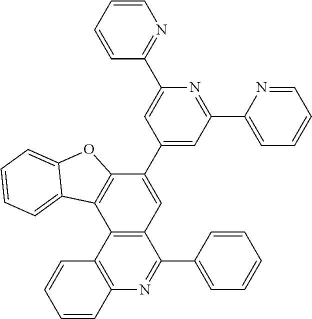 [SMILES: c1ccc(-c2nc3ccccc3c3c2cc(-c2cc(-c4ccccn4)nc(-c4ccccn4)c2)c2oc4ccccc4c23)cc1] |
| 56 | 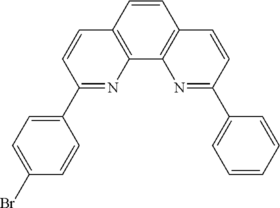 | 91 | 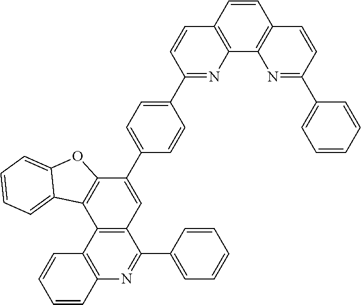 [SMILES: c1ccc(-c2ccc3ccc4ccc(-c5ccc(-c6cc7c(-c8ccccc8)nc8ccccc8c7c7c6oc6ccccc67)cc5)nc4c3n2)cc1] |
| 61 | 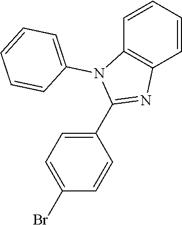 | 86 | 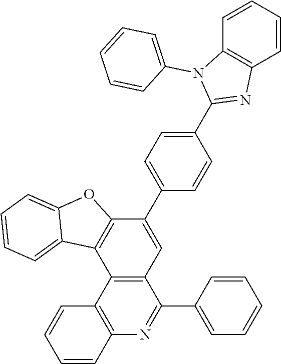 [SMILES: c1ccc(-c2nc3ccccc3c3c2cc(-c2ccc(-c4nc5ccccc5n4-c4ccccc4)cc2)c2oc4ccccc4c23)cc1] |
| 431 | 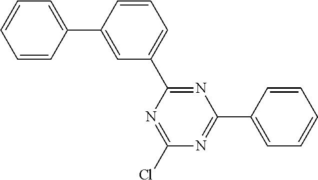 | 81 | 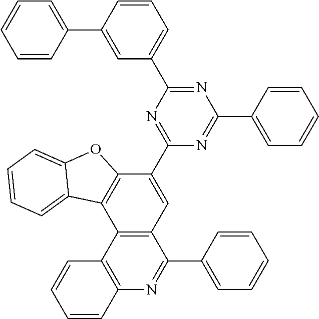 [SMILES: c1ccc(-c2cccc(-c3nc(-c4ccccc4)nc(-c4cc5c(-c6ccccc6)nc6ccccc6c5c5c4oc4ccccc45)n3)c2)cc1] |
| 433 | 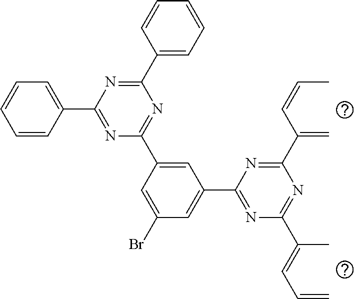 | 94 | 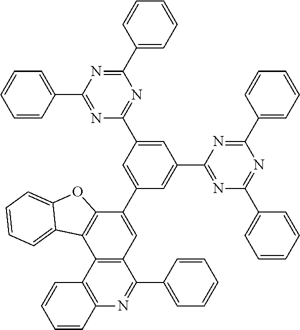 [SMILES: c1ccc(-c2nc(-c3ccccc3)nc(-c3cc(-c4nc(-c5ccccc5)nc(-c5ccccc5)n4)cc(-c4cc5c(-c6ccccc6)nc6ccccc6c5c5c4oc4ccccc45)c3)n2)cc1] |

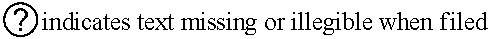

<Preparation of Core 2-1>

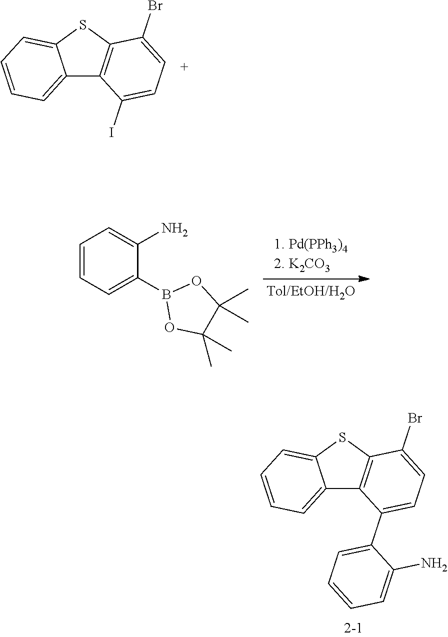 [SMILES: Brc1ccc(I)c2c1sc1ccccc12 ; CC1(C)OB(c2ccccc2N)OC1(C)C ; Nc1ccccc1-c1ccc(Br)c2sc3ccccc3c12]

A 2 L round-bottom flask was charged with 55.1 g (141.63 mmol, 1 eq.) of 1-bromo-4-iododibenzothiophene, 31.1 g (141.63 mmol, 1 eq.) of 2-amino-phenylpinacolborane, 8.2 g (7.08 mmol, 0.05 eq.) of Pd(PPh₃)₄, and 58.7 g (424.88 mmol, 3 eq.) of K₂CO₃, then toluene/EtOH/H₂O (500 ml/100 ml/100 ml) was added thereto, and the mixture was stirred under reflux.

After completion of the reaction, the mixture was extracted with MC/H₂O and the MC layer was dried over MgSO₄. Purification was performed using a silica-gel column to obtain 28.6 g of Core 2-1 at a yield of 57%.

<Preparation of Core 2-2>

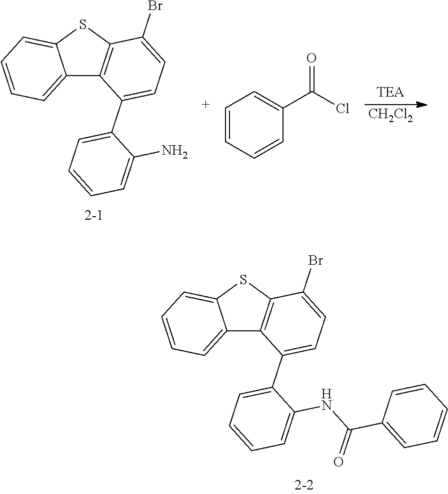

A 2 L round-bottom flask was charged with 28.6 g (80.72 mmol, 1 eq.) of Core 2-1, and 13.5 ml (96.87 mmol, 1.2 eq.) of triethanolamine (TEA), and the mixture was dissolved in 800 ml of dichloromethane (CH₂Cl₂), followed by cooling to 0° C. To the cooled mixture, 10.3 ml (88.80 mmol, 1.1 eq.) of benzoylchloride was added dropwise, and the mixture was heated to room temperature and stirred.

After completion of the reaction, the mixture was extracted with MC/H₂O and the MC layer was dried over MgSO₄.

Purification was performed using a silica-gel column to obtain 35 g of Core 2-2 at a yield of 94%.

<Preparation of Core 2-3>

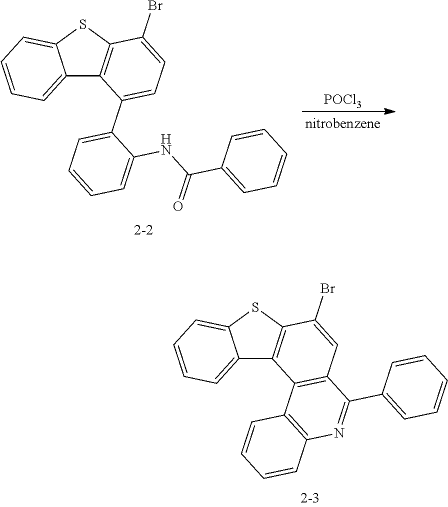 [SMILES: O=C(Nc1ccccc1-c1ccc(Br)c2sc3ccccc3c12)c1ccccc1 ; "POCl3" ; Brc1cc2c(-c3ccccc3)nc3ccccc3c2c2c1sc1ccccc12]

A 1 L round-bottom flask was charged with 35 g (75.88 mmol, 1 eq.) of Core 2-2 and 4.9 ml (53.12 mmol, 0.7 eq.) of POCl₃, then 600 ml of nitrobenzene was added thereto, and the mixture was stirred at 150° C.

After completion of the reaction, the mixture was extracted with MC/H₂O and the MC layer was dried over MgSO₄. Purification was performed using a silica-gel column to obtain 32.4 g of Core 2-3 at a yield of 97%.

<Preparation of Core 2-4>

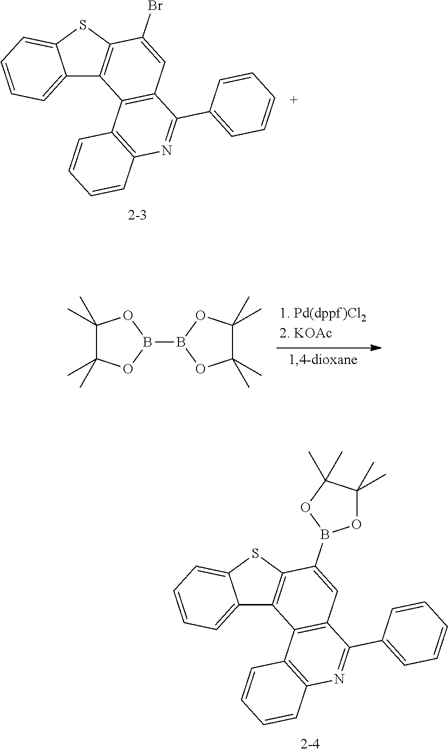

A 500 ml round-bottom flask was charged with 32 g (72.67 mmol, 1 eq.) of Core 2-3, 27.7 g (109.00 mmol, 1.5 eq.) of [bis(pinacolato)diboron], 2.7 g (3.63 mmol, 0.05 eq.) of Pd(dppf)Cl₂, and 28.5 g (290.67 mmol, 4 eq.) of potassium acetate (KOAc), then 350 ml of 1,4-dioxane was added thereto, and the mixture was stirred under reflux.

After completion of the reaction, the mixture was extracted with MC/H₂O and the MC layer was dried over MgSO₄. Purification was performed using a silica-gel column to obtain 25.5 g of Core 2-4 at a yield of 72%.

<General Procedure B>

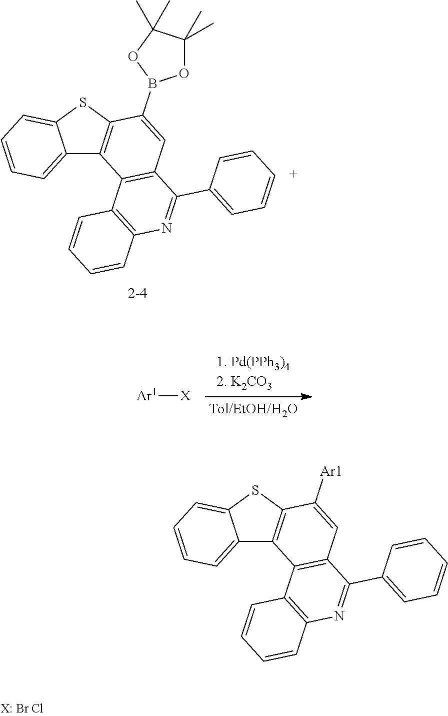

A 500 ml round-bottom flask was charged with 7 g of Core 2-4 (14.85 mmol, 1.1 eq.), Ar1-X (1 eq.) described in Table 2 below, Pd(PPh₃)₄ (0.05 eq.) and K₂CO₃ (3 eq.), then toluene/EtOH/H₂O was added thereto, and the mixture was stirred under reflux.

After completion of the reaction, the mixture was extracted with MC/H₂O and the MC layer was dried over MgSO₄. Purification was performed using a silica-gel column to obtain a material.

TABLE 2

| Com- | | Yield | |
|---|---|---|---|
| pound | Ar1—X | (%) | Structure |
| 162 | 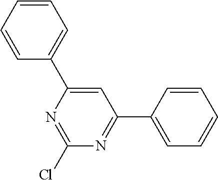 | 79 | 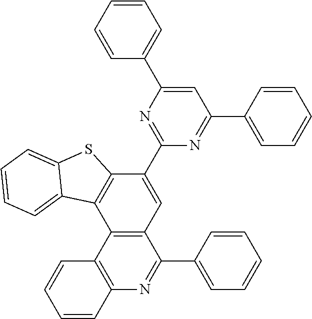 [SMILES: c1ccc(-c2cc(-c3ccccc3)nc(-c3cc4c(-c5ccccc5)nc5ccccc5c4c4c3sc3ccccc34)n2)cc1] |
| 166 | 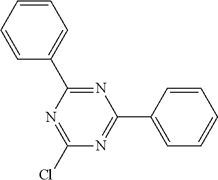 | 85 | 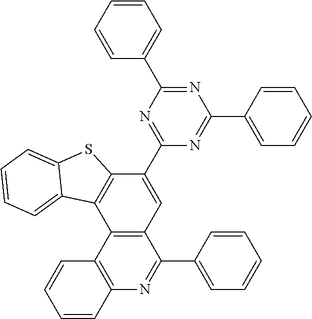 [SMILES: c1ccc(-c2nc(-c3ccccc3)nc(-c3cc4c(-c5ccccc5)nc5ccccc5c4c4c3sc3ccccc34)n2)cc1] |
| 168 | 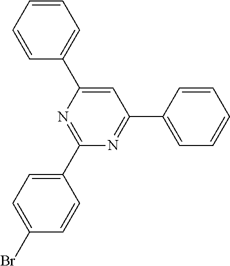 | 76 | 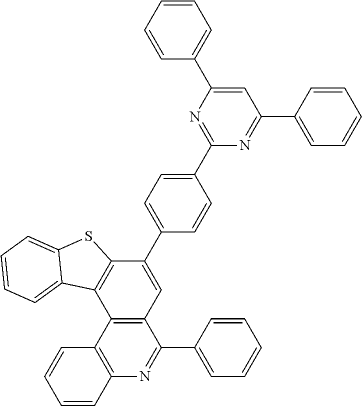 [SMILES: c1ccc(-c2cc(-c3ccccc3)nc(-c3ccc(-c4cc5c(-c6ccccc6)nc6ccccc6c5c5c4sc4ccccc45)cc3)n2)cc1] |
| 169 | 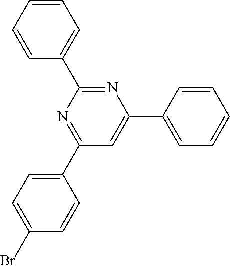 | 81 | 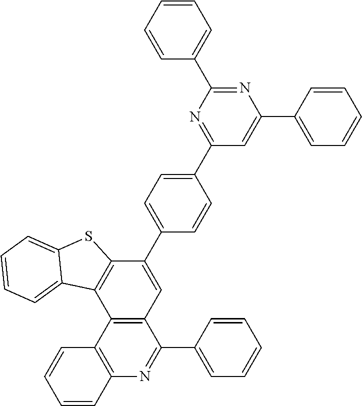 [SMILES: c1ccc(-c2cc(-c3ccc(-c4cc5c(-c6ccccc6)nc6ccccc6c5c5c4sc4ccccc45)cc3)nc(-c3ccccc3)n2)cc1] |
| 171 | 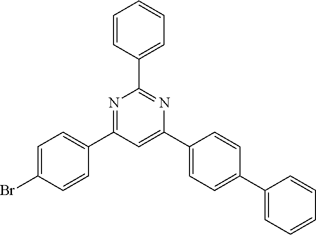 | 73 | 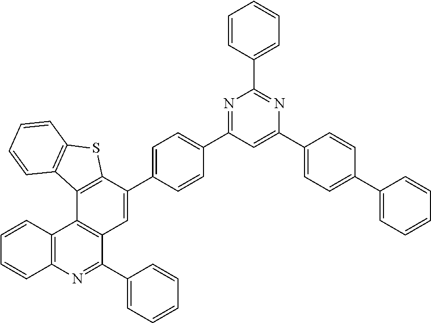 [SMILES: c1ccc(-c2ccc(-c3cc(-c4ccc(-c5cc6c(-c7ccccc7)nc7ccccc7c6c6c5sc5ccccc56)cc4)nc(-c4ccccc4)n3)cc2)cc1] |
| 172 | 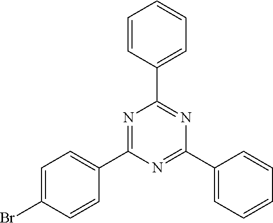 | 89 | 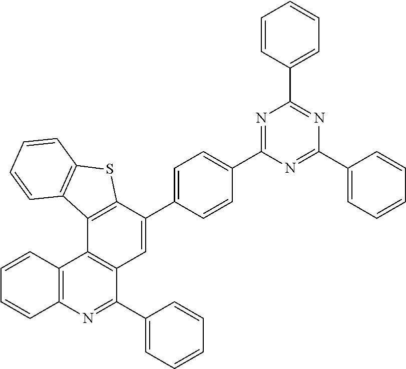 [SMILES: c1ccc(-c2nc(-c3ccccc3)nc(-c3ccc(-c4cc5c(-c6ccccc6)nc6ccccc6c5c5c4sc4ccccc45)cc3)n2)cc1] |
| 174 | 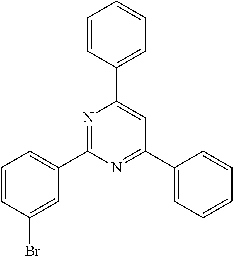 | 83 | 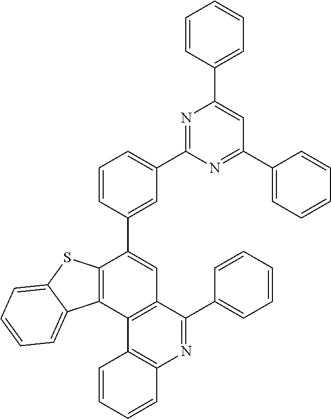 [SMILES: c1ccc(-c2cc(-c3ccccc3)nc(-c3cccc(-c4cc5c(-c6ccccc6)nc6ccccc6c5c5c4sc4ccccc45)c3)n2)cc1] |
| 178 | 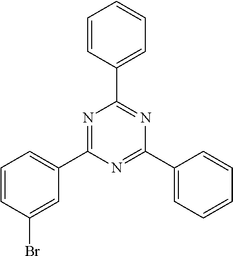 | 81 | 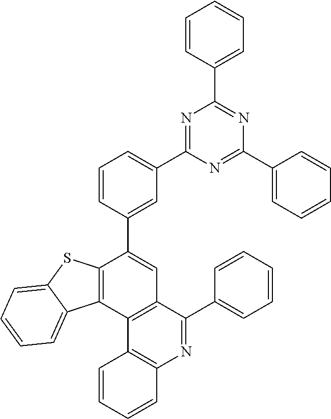 [SMILES: c1ccc(-c2nc(-c3ccccc3)nc(-c3cccc(-c4cc5c(-c6ccccc6)nc6ccccc6c5c5c4sc4ccccc45)c3)n2)cc1] |
| 184 | 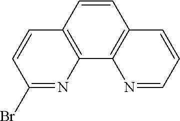 | 89 | 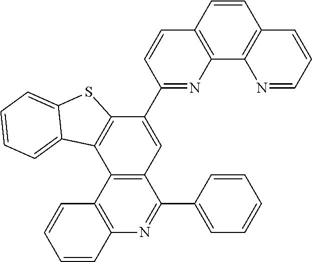 [SMILES: c1ccc(-c2nc3ccccc3c3c2cc(-c2ccc4ccc5cccnc5c4n2)c2sc4ccccc4c23)cc1] |
| 186 | 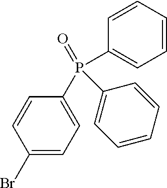 | 93 | 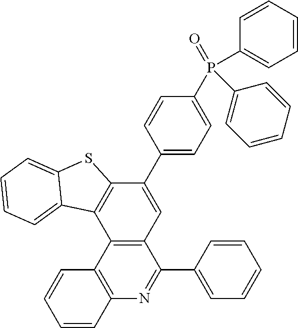 [SMILES: O=P(c1ccccc1)(c1ccccc1)c1ccc(-c2cc3c(-c4ccccc4)nc4ccccc4c3c3c2sc2ccccc23)cc1] |
| 187 | 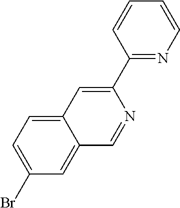 | 83 | 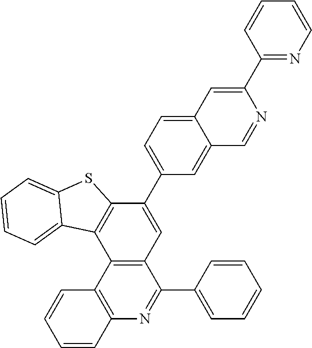 [SMILES: c1ccc(-c2nc3ccccc3c3c2cc(-c2ccc4cc(-c5ccccn5)ncc4c2)c2sc4ccccc4c23)cc1] |
| 188 | 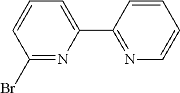 | 79 | 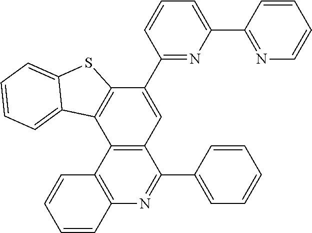 [SMILES: c1ccc(-c2nc3ccccc3c3c2cc(-c2cccc(-c4ccccn4)n2)c2sc4ccccc4c23)cc1] |
| 191 | 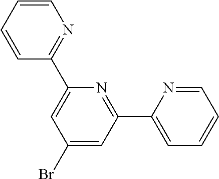 | 67 | 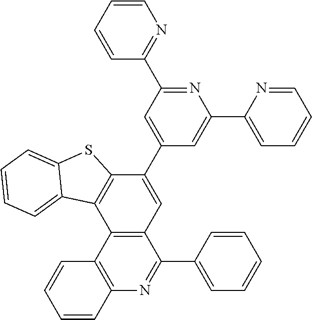 [SMILES: c1ccc(-c2nc3ccccc3c3c2cc(-c2cc(-c4ccccn4)nc(-c4ccccn4)c2)c2sc4ccccc4c23)cc1] |
| 199 | 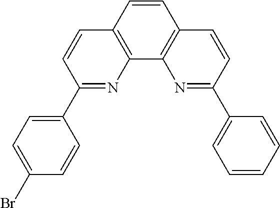 | 83 | 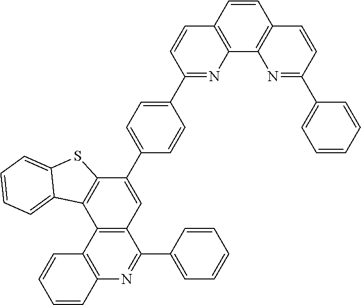 [SMILES: c1ccc(-c2ccc3ccc4ccc(-c5ccc(-c6cc7c(-c8ccccc8)nc8ccccc8c7c7c6sc6ccccc67)cc5)nc4c3n2)cc1] |
| 204 | 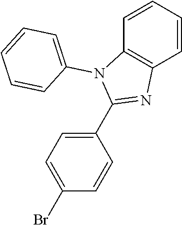 | 76 | 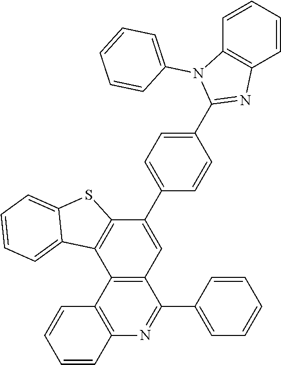 [SMILES: c1ccc(-c2nc3ccccc3c3c2cc(-c2ccc(-c4nc5ccccc5n4-c4ccccc4)cc2)c2sc4ccccc4c23)cc1] |

The compounds were prepared in the same manner as in the above Preparation Examples, and results obtained by confirming the synthesis are shown in Tables 3 and 4 below.

TABLE 3

| Compound | 1H NMR (CDC13), 300 MHz) |
|---|---|
| 19 | 8.30(2H, d), 8.23(1H, s), 8.09-8.06(2H, m), 7.98(1H, m), 7.89(1H, d), |
| | 7.79-7.78(5H, m), 7.60-7.32(13H, m) |
| 23 | 8.30-8.28(6H, m), 8.09-8.06(2H, m), 7.98(1H, m), 7.89(1H, m), 7.78(1H, m), |
| | 7.66-7.32(13H, m) |
| 25 | 8.30(2H, d), 8.23(1H, s), 8.09-8.06(2H, m), 7.98(1H, d), 7.89-7.78(8H, m), |
| | 7.66-7.32(15H, m) |
| 26 | 8.30-8.23(7H, m), 8.09-8.06(2H, m), 7.98(1H, d), 7.89(1H, d), |
| | 7.79-7.78(3H, m), 7.60-7.32(15H, m) |
| 28 | 8.30-8.23(9H, m), 8.09-8.06(2H, m), 7.98(1H, d), 7.89-7.78(4H, m), |
| | 7.66-7.25(1H, m) |
| 29 | 8.30-8.28(6H, m), 8.09-8.06(2H, m), 7.98(1H, d), 7.89-7.78(4H, m), |
| | 7.66-7.25(15H, m) |
| 31 | 8.30-8.23(4H, m), 8.09-8.06(2H, m), 7.98(1H, d), 7.89(1H, d), |
| | 7.79-7.78(5H, m), 7.66-7.32(16H, m) |
| 35 | 8.30-8.24(7H, m), 8.09-8.06(2H, m), 7.98(1H, d), 7.89(1H, d), 7.78(1H, m), |
| | 7.66-7.32(16H, m) |
| 41 | 8.83(1H, d), 8.60(1H, s), 8.48(1H, d), 8.30(2H, d), 8.10-7.98(4H, m), |
| | 7.81-7.78(3H, m), 7.66-732(9H, m) |
| 43 | 8.30(2H, d), 8.09-8.06(2H, m), 7.98(1H, d), 7.89-7.77(10H, m), |
| | 7.66-7-32(13H, m) |
| 44 | 9.30(1H, d), 8.91(1H, s), 8.62(1H, s), 8.53(1H, d), 8.30(2H, d), |
| | 8.09-7.60(4H, m), 7.84-7.32(12H, m), 7.14(1H, m) |
| 45 | 9.30(1H, d), 8.90(1H, d), 8.60-8.53(2H, m), 8.30(2H, d), 8.06-7.89(3H, m), |
| | 7.78-7.32(10H, m), 7.14(1H, m), 7.00(1H, d) |
| 48 | 9.30(2H, d), 9.15(2H, s), 8.53(2H, d), 8.30(2H, d), 8.09-7.89(4H, m), |
| | 7.78-7.32(10H, m), 7.14(2H, m) |
| 56 | 8.81(2H, d), 8.30(4H, m), 8.10-7.98(6H, m), 7.89(1H, d), 7.81-7.78(2H, m), |
| | 7.66-7.28(14H, m) |
| 61 | 8.56(1H, d), 8.30(2H, d), 8.09-7.98(3H, m), 7.89-7.78(4H, m), |
| | 7.66-7.22(17H, m) |
| 162 | 8.45(1H, d), 8.30(2H, d), 8.23(1H, s), 8.09-8.06(2H, m), 7.98(2H, m), |
| | 7.79-7.78(5H, m), 7.60-7.41(12H, m) |
| 166 | 8.45(1H, d), 8.30-8.28(6H, m), 8.09-8.06(2H, m), 7.98(2H, m), 7.78(1H, m), |
| | 7.60-7.41(12H, m) |
| 168 | 8.45(1H, d), 8.30(2H, d), 8.23(1H, s), 8.09-8.06(2H, m), 7.98(2H, m), |
| | 7.85-7.79(7H, m), 7.60-7.41(12H, m), 7.25(2H, dd) |
| 169 | 8.45(1H, d), 830-8.23(7H, m), 8.09-8.06(2H, m), 7.98(2H, m), |
| | 7.79-7.78(3H, m), 7.60-7.41(12H, m), 7.25(2H, dd) |
| 171 | 8.45(1H, d), 8.30-8.23(9H, m), 8.09-8.06(2H, m), 7.98(2H, m), |
| | 7.85-7.78(3H, m), 7.60-7.41(14H, m), 7.25(2H, dd) |
| 172 | 8.45(1H, d), 8.30-8.28(6H, m), 8.09-8.06(2H, m), 7.98(2H, m), |
| | 7.85-7.78(3H, m), 7.60-7.41(12H, m), 7.25(2H, dd) |
| 174 | 8.45(1H, d), 830-8.23(4H, m), 8.09-8.06(2H, m), 7.98(2H, m), |
| | 7.79-7.78(5H, m), 7.70(1H, s), 7.60-7.41 (14H, m) |
| 178 | 8.45(1H, d), 830-8.24(7H, m), 8.09-8.06(2H, m), 7.98(2H, m), 7.78(1H, m), |
| | 7.70(1H, s), 7.60-7.41 (14H, m) |
| 184 | 8.83(1H, d), 8.60(1H, s), 8.48-8.45(2H, m), 8.30(2H, d), 8.10-7.98(5H, m), |
| | 7.81-7.78(2H, m), 7.60-7.50(7H, m), 7.35(1H, d) |
| 186 | 8.45(1H, d), 8.30(2H, d), 8.09-8.06(2H, m), 7.98(2H, m), 7.83-7.77(9H, m), |
| | 7.60-7.45(12H, m) |
| 187 | 9.30(1H, d), 8.91(1H, s), 8.62(1H, s), 8.53-8.45(2H, m), 8.30(2H, d), |
| | 8.09-7.98(5H, m), 7.84-7.50(10H, m), 7.14(1H, m) |
| 188 | 9.30(1H, d), 8.90(1H, d), 8.60-8.45(3H, m), 8.30(2H, d), 8.06-7.98(3H, m), |
| | 7.78-7.50(9H, m), 7.14(1H, m), 7.00(1H, d) |
| 191 | 9.30(2H, d), 9.15(2H, s), 8.53-8.45(3H, m), 8.30(2H, d), 8.09-7.98(4H, m), |
| | 7.78-7.47(9H, m), 7.14(2H, m) |
| 199 | 8.81(2H, dd), 8.45(1H, d), 8.30(4H, m), 8.10-7.98(7H, m), 7.81-7.78(2H, m), |
| | 7.60-7.47(9H, m), 735-7.28(4H, m) |
| 204 | 8.56(1H, d), 8.45(1H, d), 8.30(2H, d), 8.09-7.98(4H, m), 7.85-7.78(3H, m), |
| | 7.60-7.45(12H, m), 7.25-7.22(4H, m) |
| 431 | 8.30-8.24(5H, m), 8.09-8.06(2H, m), 7.98(1H, d), 7.89(1H, d), 7.78(1H, m), |
| | 7.60-7.32(18H, m) |
| 433 | 8.30-8.28(10H, m), 8.09-8.06(2H, m), 7.98(1H, d), 7.89(1H, d), 7.89(1H, d), |
| | 7.78(1H, m), 7.66-7.32(22H, m) |

TABLE 4

| Compound | FD-MS | Compound | FD-MS |
|---|---|---|---|
| 1 | m/z = 421.15C31H19NO = | 2 | m/z = 471.16G35H21NO = |
| | 421.50 | | 471.56 |
| 3 | m/z = 521.18C39H23NO = | 4 | m/z = 597.21C45H27NO = |
| | 521.62 | | 597.72 |
| 5 | m/z = 647.22C49H29NO = | 6 | m/z = 545.18C41H23NO = |
| | 647.78 | | 545.64 |
| 7 | m/z = 571.19C43H25NO = | 8 | m/z = 573.21C43H27NO = |
| | 571.68 | | 573.70 |
| 9 | m/z = 527.13C37H21NOS = | 10 | m/z = 527.13C37H21NOS = |
| | 527.64 | | 527.64 |
| 11 | m/z = 511.16C37H21NO2 = | 12 | m/z = 511.16C37H21NO2 = |
| | 511.58 | | 511.58 |
| 13 | m/z = 422.14C30H18N2O = | 14 | m/z = 422.14C30H18N2O = |
| | 422.49 | | 422.49 |
| 15 | m/z = 422.14C30H18N2O = | 16 | m/z = 423.14C29H17N3O = |
| | 422.49 | | 423.48 |
| 17 | m/z = 423.14C29H17N3O = | 18 | m/z = 423.14C29H17N3O = |
| | 423.48 | | 423.48 |
| 19 | m/z = 575.20C41H25N3O = | 20 | m/z = 575.20C41H25N3O = |
| | 575.67 | | 575.67 |
| 21 | m/z = 651.23C47H29N3O = | 22 | m/z = 651.23C47H29N3O = |
| | 651.77 | | 651.77 |
| 23 | m/z = 576.20C40H24N4O = | 24 | m/z = 652.23C46H28N4O = |
| | 576.6 | | 652.76 |
| 25 | m/z = 651.23C47H29N3O = | 26 | m/z = 651.23C47H29N3O = |
| | 651.77 | | 651.77 |
| 27 | m/z = 727.26C53H33N3O = | 28 | m/z = 727.26C53H33N3O = |
| | 727.87 | | 27.87 |
| 29 | m/z = 652.23C46H28N4O = | 30 | m/z = 728.26C52H32N4O = |
| | 652.76 | | 728.86 |
| 31 | m/z = 651.23 C47H29N3O | 32 | m/z = 651.23C47H29N3O = |
| | 651.77= | | 651.77 |
| 33 | m/z = 727.26C53H33N3O = | 34 | m/z = 727.26C53H33N3O = |
| | 727.87 | | 727.87 |
| 35 | m/z = 652.23 C46H28N4O = | 36 | m/z = 728.26C52H32N4O = |
| | 652.76 | | 728.86 |
| 37 | m/z = 472.16C34H20N2O = | 38 | m/z = 472.16C34H20N2O = |
| | 472.55 | | 472.55 |
| 39 | m/z = 472.16C34H20N2O = | 40 | m/z = 472.16C34H20N2O = |
| | 472.55 | | 472.55 |
| 41 | m/z = 523.17C37H21N3O = | 42 | m/z = 523.17C37H21N3O = |
| | 523.60 | | 523.60 |
| 43 | m/z = 621.19C43H28NO2P = | 44 | m/z = 549.18C39H23N3O = |
| | 621.68 | | 549.63 |
| 45 | m/z = 499.17C35H21N3O = | 46 | m/z = 499.17C35H21N3O = |
| | 499.5 | | 499.57 |
| 47 | m/z = 499.17C35H21N3O = | 48 | m/z = 576.20C40H24N4O = |
| | 499.57 | | 576.66 |
| 49 | m/z = 549.18C39H23N3O = | 50 | m/z = 625.22C45H27N3O = |
| | 549.63 | | 625.73 |
| 51 | m/z = 625.22C45H27N3O = | 52 | m/z = 549.18C39H23N3O = |
| | 625.73 | | 549.63 |
| 53 | m/z = 625.22C45H27N3O = | 54 | m/z = 625.22C45H27N3O = |
| | 625.73 | | 625.73 |
| 55 | m/z= 599.20C43H25N30 = | 56 | m/z = 675.23C49H29N3O = |
| | 599.69 | | 675.79 |
| 57 | m/z = 675.23C49H29N3O = | 58 | m/z = 489.18C34H23N3O = |
| | 675.79 | | 489.58 |
| 59 | m/z = 537.18C38H23N3O = | 60 | m/z = 565.22C40H27N3O = |
| | 537.62 | | 565.68 |
| 61 | m/z = 613.22C44H27N3O = | 62 | m/z = 613.22C44H27N3O = |
| | 613.72 | | 613.72 |
| 63 | m/z = 510.17C37H22N2O = | 64 | m/z = 586.20C43H26N2O = |
| | 510.60 | | 586.69 |
| 65 | m/z = 586.20C43H26N2O = | 66 | m/z = 586.20C43H26N2O = |
| | 586.69 | | 586.69 |
| 67 | m/z = 586.20C43H26N2O = | 68 | m/z = 586.20C43H26N2O = |
| | 586.69 | | 586.69 |
| 69 | m/z = 586.20C43H26N20 = | 70 | m/z = 537.21C40H27NO = |
| | 586.69 | | 537.66 |
| 71 | m/z = 661.24C50H31NO = | 72 | m/z = 659.22C50H29NO = |
| | 661.80 | | 659.79 |
| 73 | m/z = 659.22C50H29NO = | 74 | m/z = 659.22C50H29NO = |
| | 659.79 | | 659.79 |
| 75 | m/z = 552.22C40H28N2O = | 76 | m/z = 628.25C46H32N2O = |
| | 552.68 | | 628.78 |
| 77 | m/z = 628.25C46H32N2O = | 78 | m/z = 628.25C46H32N2O = |
| | 628.78 | | 628.78 |
| 79 | m/z = 628.25C46H32N2O = | 80 | m/z = 676.25C50H32N2O = |
| | 628.78 | | 676.82 |
| 81 | m/z = 752.28C56H36N2O = | 82 | m/z = 752.28C56H36N2O = |
| | 752.92 | | 752.92 |
| 83 | m/z = 752.28C56H36N2O = | 84 | m/z = 752.28C56H36N2O = |
| | 752.92 | | 752.92 |
| 85 | m/z = 674.24C50H30N2O = | 86 | m/z = 750.27C56H34N2O = |
| | 674.80 | | 750.90 |
| 87 | m/z = 750.27C56H34N2O = | 88 | m/z = 750.27C56H34N2O = |
| | 750.90 | | 750.90 |
| 89 | m/z = 750.27C56H34N2O = | 90 | m/z = 526.17C37H22N2O2 = |
| | 750.90 | | 526.60 |
| 91 | m/z = 602.20C43H26N2O2 = | 92 | m/z = 602.20C43H26N2O2 = |
| | 602.69 | | 602.69 |
| 93 | m/z = 602.20C43H26N2O2 = | 94 | m/z = 602.20C43H26N2O2 = |
| | 602.69 | | 602.69 |
| 95 | m/z = 542.15C37H22N2OS = | 96 | m/z = 618.18C43H26N2OS = |
| | 542.66 | | 618.75 |
| 97 | m/z = 618.18C43H26N2OS = | 98 | m/z = 618.18C43H26N2OS = |
| | 618.75 | | 618.75 |
| 99 | m/z = 618.18C43H26N2OS = | 100 | m/z = 584.19C43H24N2O = |
| | 618.75 | | 584.68 |
| 101 | m/z = 660.22C49H28N2O = | 102 | m/z = 660.22 C49H28N2O = |
| | 660.78 | | 660.78 |
| 103 | m/z = 660.22C49H28N2O = | 104 | m/z = 751.26C55H33N3O = |
| | 660.78 | | 751.89 |
| 105 | m/z = 512.19C37H24N2O = | 106 | m/z = 588.22C43H28N2O = |
| | 512.61 | | 588.71 |
| 107 | m/z = 664.25C49H32N2O = | 108 | m/z = 588.22C43H28N2O = |
| | 664.81 | | 588.71 |
| 109 | m/z = 664.25C49H32N2O = | 110 | m/z = 664.25C49H32N2O = |
| | 664.81 | | 664.81 |
| 111 | m/z = 628.25C46H32N2O = | 112 | m/z = 704.28C52H36N2O = |
| | 628.78 | | 704.87 |
| 113 | m/z = 744.31C55H40N2O = | 114 | m/z = 562.20C41H26N2O = |
| | 744.94 | | 562.67 |
| 115 | m/z = 612.22C45H28N2O = | 116 | m/z = 638.24C47H30N2O = |
| | 612.73 | | 638.77 |
| 117 | m/z = 638.24C47H30N2O = | 118 | m/z = 678.27C50H34N2O = |
| | 638.77 | | 678.84 |
| 119 | m/z = 562.20C41H26N2O = | 120 | m/z = 612.22C45H28N2O = |
| | 562.67 | | 612.73 |
| 121 | m/z = 638.24C47H30N2O = | 122 | m/z = 638.24C47H30N2O = |
| | 638.77 | | 638.77 |
| 123 | m/z = 678.27C50H34N2O = | 124 | m/z = 751.26C55H33N3O = |
| | 678.84 | | 751.89 |
| 125 | m/z = 827.29C61H37N3O = | 126 | m/z = 827.29C61H37N3O = |
| | 827.99 | | 827.99 |
| 127 | m/z = 801.28C59H35N3O = | 128 | m/z = 801.28C59H35N3O = |
| | 801.95 | | 801.95 |
| 129 | m/z = 903.32C67H41N3O = | 130 | m/z = 875.29C65H37N3O = |
| | 904.09 | | 876.03 |
| 131 | m/z = 901.31C67H39N3O = | 132 | m/z = 692.19C49H28N2OS = |
| | 902.07 | | 692.84 |
| 133 | m/z = 676.22C49H28N2O2 = | 134 | m/z =702.27C52H34N2O = |
| | 676.78 | | 702.86 |
| 135 | m/z = 826.30C62H38N2O = | 136 | m/z = 824.28C62H36N2O = |
| | 827.00 | | 824.98 |
| 137 | m/z = 840.28C62H36N2O2 = | 138 | m/z = 856.25C62H36N2OS = |
| | 840.98 | | 857.04 |
| 139 | m/z = 915.32C68H41N3O = | 140 | m/z = 692.19C49H28N2OS = |
| | 916.10 | | 692.84 |
| 141 | m/z = 676.22C49H28N2O2 = | 142 | m/z = 778.30C58H38N2O = |
| | 676.78 | | 778.96 |
| 143 | m/z = 827.29C61H37N3O = | 144 | m/z = 437.12C31H19NS = |
| | 827.99 | | 437.56 |
| 145 | m/z = 487.14C35H21NS = | 146 | m/z = 537.16C39H23NS = |
| | 487.62 | | 537.68 |
| 147 | m/z = 613.19C45H27NS = | 148 | m/z = 663.20C49H29NS = |
| | 613.78 | | 663.84 |
| 149 | m/z = 561.16C41H23NS = | 150 | m/z = 587.17C43H25NS = |
| | 561.70 | | 587.74 |
| 151 | m/z = 589.19C43H27NS = | 152 | m/z = 543.11C37H21NS2 = |
| | 589.7 | | 543.70 |
| 153 | m/z = 543.11C37H21NS2 = | 154 | m/z = 527.13C37H21NOS = |
| | 543.70 | | 527.64 |
| 155 | m/z = 527.13C37H21NOS = | 156 | m/z = 438.12C30H18N2S = |
| | 527.64 | | 438.55 |
| 157 | m/z = 438.12C30H18N2S = | 158 | m/z = 438.12C30H18N2S = |
| | 438.55 | | 438.55 |
| 159 | m/z = 439.11C29H17N3S = | 160 | m/z = 439.11C29H17N3S = |
| | 439.54 | | 439.54 |
| 161 | m/z = 439.11C29H17N3S = | 162 | m/z = 591.18C41H25N3S = |
| | 439.54 | | 591.73 |
| 163 | m/z = 591.18C41H25N3S = | 164 | m/z = 667.21C47H29N3S = |
| | 591.73 | | 667.83 |
| 165 | m/z = 667.21C47H29N3S = | 166 | m/z = 592.17C40H24N4S = |
| | 667.83 | | 592.72 |
| 167 | m/z = 668.20C46H28N4S = | 168 | m/z = 667.21C47H29N3S = |
| | 668.82 | | 667.83 |
| 169 | m/z = 667.21C47H29N3S = | 170 | m/z = 743.24C53H33N3S = |
| | 667.83 | | 743.93 |
| 171 | m/z = 743.24C53H33N3S = | 172 | m/z = 668.20C46H28N4S = |
| | 743.93 | | 668.82 |
| 173 | m/z = 744.23C52H32N4S = | 174 | m/z = 667.21C47H29N3S = |
| | 744.92 | | 667.83 |
| 175 | m/z = 667.21C47H29N3S = | 176 | m/z = 743.24C53H33N3S = |
| | 667.83 | | 743.93 |
| 177 | m/z= 743.24C53H33N3S = | 178 | m/z = 668.20C46H28N4S = |
| | 743.93 | | 668.82 |
| 179 | m/z = 744.23C52H32N4S = | 180 | m/z = 488.13C34H20N2S = |
| | 744.92 | | 488.61 |
| 181 | m/z = 488.13C34H20N2S = | 182 | m/z = 488.13C34H20N2S = |
| | 488.61 | | 488.61 |
| 183 | m/z = 488.13C34H20N2S = | 184 | m/z = 539.15C37H21N3S = |
| | 488.61 | | 539.66 |
| 185 | m/z = 539.15C37H21N3S = | 186 | m/z = 637.16C43H28NOPS = |
| | 539.66 | | 637.74 |
| 187 | m/z = 565.16C39H23N3S = | 188 | m/z = 515.15C35H21N3S = |
| | 565.69 | | 515.63 |
| 189 | m/z=515.15C35H21N3S = | 190 | m/z = 515.15C35H21N3S = |
| | 515.63 | | 515.63 |
| 191 | m/z = 592.17C40H24N4S = | 192 | m/z = 565.16C39H23N3S = |
| | 592.72 | | 565.69 |
| 193 | m/z = 641.19C45H27N3S = | 194 | m/z = 641.19C45H27N3S = |
| | 641.79 | | 641.79 |
| 195 | m/z = 565.16C39H23N3S = | 196 | m/z = 641.19C45H27N3S = |
| | 565.69 | | 641.79 |
| 197 | m/z = 641.19C45H27N3S = | 198 | m/z = 615.18C43H25N3S = |
| | 641.79 | | 615.75 |
| 199 | m/z = 691.21C49H29N3S = | 200 | m/z = 691.21C49H29N3S = |
| | 691.85 | | 691.85 |
| 201 | m/z = 505.16C34H23N3S = | 202 | m/z = 553.16C38H23N3S = |
| | 505.64 | | 553.68 |
| 203 | m/z = 581.19C40H27N3S = | 204 | m/z = 629.19C44H27N3S = |
| | 581.74 | | 629.78 |
| 205 | m/z = 629.19C44H27N3S = | 206 | m/z = 526.15C37H22N2S = |
| | 629.78 | | 526.66 |
| 207 | m/z = 602.18C43H26N2S = | 208 | m/z = 602.18C43H26N2S = |
| | 602.76 | | 602.76 |
| 209 | m/z = 602.18C43H26N2S = | 210 | m/z = 602.18C43H26N2S = |
| | 602.76 | | 602.76 |
| 211 | m/z = 602.18C43H26N2S = | 212 | m/z = 602.18C43H26N2S = |
| | 602.76 | | 602.76 |
| 213 | m/z = 553.19C40H27NS = | 214 | m/z = 677.22C50H31NS = |
| | 553.72 | | 677.87 |
| 215 | m/z = 675.20C50H29NS = | 216 | m/z = 675.20C50H29NS = |
| | 675.85 | | 675.85 |
| 217 | m/z = 675.20C50H29NS = | 218 | m/z = 568.20C40H28N2S = |
| | 675.85 | | 568.74 |
| 219 | m/z = 644.23C46H32N2S = | 220 | m/z = 644.23C46H32N2S = |
| | 644.84 | | 644.84 |
| 221 | m/z = 644.23C46H32N2S = | 222 | m/z = 644.23C46H32N2S = |
| | 644.84 | | 644.84 |
| 223 | m/z = 692.23C50H32N2S = | 224 | m/z = 768.26C56H36N2S = |
| | 692.88 | | 768.98 |
| 225 | m/z = 768.26C56H36N2S = | 226 | m/z = 768.26C56H36N2S = |
| | 768.98 | | 768.98 |
| 227 | m/z = 768.26C56H36N2S = | 228 | m/z = 690.21C50H30N2S = |
| | 768.98 | | 690.86 |
| 229 | m/z = 766.24C56H34N2S = | 230 | m/z = 766.24C56H34N2S = |
| | 766.96 | | 766.96 |
| 231 | m/z = 766.24C56H34N2S = | 232 | m/z = 766.24C56H34N2S = |
| | 766.96 | | 766.96 |
| 233 | m/z = 542.15C37H22N2OS = | 234 | m/z = 618.18C43H26N2OS = |
| | 542.66 | | 618.75 |
| 235 | m/z = 618.18C43H26N2OS = | 236 | m/z = 618.18C43H26N2OS = |
| | 618.75 | | 618.75 |
| 237 | m/z = 618.18C43H26N2OS = | 238 | m/z = 558.12C37H22N2S2 = |
| | 618.75 | | 558.72 |
| 239 | m/z = 634.15C43H26N2S2 = | 240 | m/z = 634.15C43H26N2S2 = |
| | 634.82 | | 634.82 |
| 241 | m/z = 634.15C43H26N2S2 = | 242 | m/z = 634.15C43H26N2S2 = |
| | 634.82 | | 634.82 |
| 243 | m/z = 600.17C43H24N2S = | 244 | m/z = 676.20C49H28N2S = |
| | 600.74 | | 676.84 |
| 245 | m/z = 676.20C49H28N2S = | 246 | m/z = 676.20C49H28N2S = |
| | 676.84 | | 676.84 |
| 247 | m/z = 767.24C55H33N3S = | 248 | m/z = 528.17C37H24N2S = |
| | 767.95 | | 528.67 |
| 249 | m/z = 604.20C43H28N2S = | 250 | m/z = 680.23C49H32N2S = |
| | 604.77 | | 680.87 |
| 251 | m/z = 604.20C43H28N2S = | 252 | m/z = 680.23C49H32N2S = |
| | 604.77 | | 680.87 |
| 253 | m/z = 680.23C49H32N2S = | 254 | m/z = 644.23C46H32N2S = |
| | 680.87 | | 644.84 |
| 255 | m/z = 720.26C52H36N2S = | 256 | m/z = 760.29C55H40N2S = |
| | 720.93 | | 761.00 |
| 257 | m/z = 578.18C41H26N2S = | 258 | m/z = 628.20C45H28N2S = |
| | 578.73 | | 628.79 |
| 259 | m/z = 654.21C47H30N2S = | 260 | m/z = 654.21C47H30N2S = |
| | 654.83 | | 654.83 |
| 261 | m/z = 694.24C50H34N2S = | 262 | m/z = 578.18C41H26N2S = |
| | 694.90 | | 578.73 |
| 263 | m/z = 628.20C45H28N2S = | 264 | m/z = 654.21C47H30N2S = |
| | 628.79 | | 654.83 |
| 265 | m/z = 654.21C47H30N2S = | 266 | m/z = 694.24C50H34N2S = |
| | 654.83 | | 694.90 |
| 267 | m/z = 767.24C55H33N3S = | 268 | m/z = 843.27C61H37N3S = |
| | 767.95 | | 844.05 |
| 269 | m/z = 843.27C61H37N3S = | 270 | m/z = 817.26C59H35N3S = |
| | 844.05 | | 818.01 |
| 271 | m/z = 817.26C59H35N3S = | 272 | m/z = 919.30C67H41N3S = |
| | 818.01 | | 920.15 |
| 273 | m/z = 891.27C65H37N3S = | 274 | m/z = 917.29C67H39N3S = |
| | 892.09 | | 918.13 |
| 275 | m/z = 708.17C49H28N2S2 = | 276 | m/z = 692.19C49H28N2OS = |
| | 708.90 | | 692.84 |
| 277 | m/z = 718.24C52H34N2S = | 278 | m/z = 842.28C62H38N2S = |
| | 718.92 | | 843.06 |
| 279 | m/z = 840.26C62H36N2S = | 280 | m/z = 856.25C62H36N2OS = |
| | 841.04 | | 857.04 |
| 281 | m/z = 872.23C62H36N2S2 = | 282 | m/z = 931.30C68H41N3S = |
| | 873.10 | | 932.16 |
| 283 | m/z = 708.17C49H28N2S2 = | 284 | m/z = 692.19C49H28N2OS = |
| | 708.90 | | 692.84 |
| 285 | m/z = 794.28C58H38N2S = | 286 | m/z = 843.27C61H37N3S = |
| | 795.02 | | 844.05 |
| 287 | m/z = 741.24C53H31N3O2 = | 288 | m/z = 666.21C46H26N4O2 = |
| | 741.85 | | 666.74 |
| 289 | m/z = 817.27C59H35N3O2 = | 290 | m/z = 742.24C52H30N4O2 = |
| | 817.95 | | 742.84 |
| 291 | m/z = 817.27C59H35N3O2 = | 292 | m/z = 613.18C43H23N3O2 = |
| | 817.95 | | 613.68 |
| 293 | m/z = 613.18C43H23N3O2 = | 294 | m/z = 711.20C49H30NO3P = |
| | 613.68 | | 711.76 |
| 295 | m/z = 676.22C49H28N2O2 = | 296 | m/z = 676.22C49H28N2O2 = |
| | 676.78 | | 676.78 |
| 297 | m/z = 757.22C53H31N3OS = | 298 | m/z = 682.18C46H26N4OS = |
| | 757.91 | | 682.80 |
| 299 | m/z = 833.25C59H35N3OS = | 300 | m/z = 758.21C52H30N4OS = |
| | 834.01 | | 758.90 |
| 301 | m/z = 833.25C59H35N3OS = | 302 | m/z = 629.16C43H23N3OS = |
| | 834.01 | | 629.74 |
| 303 | m/z = 629.16C43H23N3OS = | 304 | m/z = 727.17C49H30NO2PS = |
| | 629.74 | | 727.82 |
| 305 | m/z = 692.19C49H28N2OS = | 306 | m/z = 692.19C49H28N2OS = |
| | 692.84 | | 692.84 |
| 307 | m/z = 767.29C56H37N3O = | 308 | m/z = 692.26C49H32N4O = |
| | 767.93 | | 692.82 |
| 309 | m/z = 843.32C62H41N3O = | 310 | m/z = 768.29C55H36N4O = |
| | 844.03 | | 768.92 |
| 311 | m/z = 843.32C62H41N3O = | 312 | m/z = 639.23C46H29N3O = |
| | 844.03 | | 639.76 |
| 313 | m/z = 639.23C46H29N3O = | 314 | m/z = 737.25C52H36NO2P = |
| | 639.76 | | 737.84 |
| 315 | m/z = 702.27C52H34N2O = | 316 | m/z = 702.27C52H34N2O = |
| | 702.86 | | 702.86 |
| 317 | m/z = 816.29C59H36N4O = | 318 | m/z = 741.25C52H31N5O = |
| | 816.96 | | 741.85 |
| 319 | m/z = 892.32C65H40N4O = | 320 | m/z = 817.28C58H35N5O = |
| | 893.06 | | 817.95 |
| 321 | m/z = 892.32C65H40N4O = | 322 | m/z = 688.23C49H28N4O = |
| | 893.06 | | 688.79 |
| 323 | m/z = 688.23C49H28N4O = | 324 | m/z = 786.24C55H35N2O2P = |
| | 688.79 | | 786.87 |
| 325 | m/z = 751.26C55H33N3O = | 326 | m/z = 751.26C55H33N3O = |
| | 751.89 | | 751.89 |
| 327 | m/z = 816.29C59H36N4O = | 328 | m/z = 763.26C56H33N3O = |
| | 816.96 | | 763.90 |
| 329 | m/z = 861.28C62H40NO2P = | 330 | m/z = 826.30C62H38N2O = |
| | 861.98 | | 827.00 |
| 331 | m/z = 814.27C59H34N4O = | 332 | m/z = 761.25C56H31N3O = |
| | 814.95 | | 761.88 |
| 333 | m/z = 859.26C62H38NO2P = | 334 | m/z = 824.28C62H36N2O = |
| | 859.96 | | 824.98 |
| 335 | m/z = 841.27C61H35N3O2 = | 336 | m/z = 857.25C61H35N3OS = |
| | 841.97 | | 858.03 |
| 337 | m/z = 916.32C67H40N4O = | 338 | m/z = 867.32C64H41N3O = |
| | 917.08 | | 868.05 |
| 339 | m/z = 991.36C74H45N3O = | 340 | m/z = 989.34C74H43N3O = |
| | 992.19 | | 990.18 |
| 341 | m/z = 689.21C49H27N3O2 = | 342 | m/z = 765.24C55H31N3O2 = |
| | 689.77 | | 765.87 |
| 343 | m/z = 703.23C50H29N3O2 = | 344 | m/z = 705.19C49H27N3OS = |
| | 703.80 | | 705.84 |
| 345 | m/z = 781.22C55H31N3OS = | 346 | m/z = 719.20C50H29N3OS = |
| | 781.93 | | 719.86 |
| 347 | m/z = 764.26C55H32N4O = | 348 | m/z = 840.29C61H36N4O = |
| | 764.89 | | 840.99 |
| 349 | m/z = 778.27C56H34N4O = | 350 | m/z = 715.26C52H33N3O = |
| | 778.92 | | 715.86 |
| 351 | m/z = 791.29C58H37N3O = | 352 | m/z = 729.28C53H35N3O = |
| | 791.95 | | 729.88 |
| 353 | m/z = 839.29C62H37N3O = | 354 | m/z = 915.32C68H41N3O = |
| | 840.00 | | 916.10 |
| 355 | m/z = 853.31C63H39N3O = | 356 | m/z = 837.28C62H35N3O = |
| | 854.03 | | 837.98 |
| 357 | m/z = 913.31C68H39N3O = | 358 | m/z = 851.29C63H37N3O = |
| | 914.08 | | 852.01 |
| 359 | m/z = 757.22C53H31N3OS = | 360 | m/z = 682.18C46H26N4OS = |
| | 757.91 | | 682.80 |
| 361 | m/z = 833.25C59H35N3OS = | 362 | m/z = 758.21C52H30N4OS = |
| | 834.01 | | 758.90 |
| 363 | m/z = 832.25C60H36N2OS = | 364 | m/z = 629.16C43H23N3OS = |
| | 833.02 | | 629.74 |
| 365 | m/z = 629.16C43H23N3OS = | 366 | m/z = 727.17C49H30NO2PS = |
| | 629.74 | | 727.82 |
| 367 | m/z = 692.19C49H28N2OS = | 368 | m/z = 692.19C49H28N2OS = |
| | 692.84 | | 692.84 |
| 369 | m/z = 773.20C53H31N3S2 = | 370 | m/z = 698.16C46H26N4S2 = |
| | 773.97 | | 698.86 |
| 371 | m/z = 849.23C59H35N3S2 = | 372 | m/z = 774.19C52H30N4S2 = |
| | 850.07 | | 774.96 |
| 373 | m/z = 849.23C59H35N3S2 = | 374 | m/z = 645.13C43H23N3S2 = |
| | 850.07 | | 645.80 |
| 375 | m/z = 645.13C43H23N3S2 = | 376 | m/z = 743.15C49H30NOPS2 = |
| | 645.80 | | 743.88 |
| 377 | m/z = 708.17C49H28N2S2 = | 378 | m/z = 708.17C49H28N2S2 = |
| | 708.90 | | 708.90 |
| 379 | m/z = 783.27C56H37N3S = | 380 | m/z = 708.23C49H32N4S = |
| | 783.99 | | 708.88 |
| 381 | m/z = 859.30C62H41N3S = | 382 | m/z = 784.27C55H36N4S = |
| | 860.09 | | 784.98 |
| 383 | m/z = 859.30C62H41N3S = | 384 | m/z = 655.21C46H29N3S = |
| | 860.09 | | 655.82 |
| 385 | m/z = 655.21C46H29N3S = | 386 | m/z = 753.23C52H36NOPS = |
| | 655.82 | | 753.90 |
| 387 | m/z = 718.24C52H34N2S = | 388 | m/z = 718.24C52H34N2S = |
| | 718.92 | | 718.92 |
| 389 | m/z = 832.27C59H36N4S = | 390 | m/z = 757.23C52H31N5S = |
| | 833.03 | | 757.92 |
| 391 | m/z = 908.30C65H40N4S = | 392 | m/z = 833.26C58H35N5S = |
| | 909.12 | | 834.01 |
| 393 | m/z = 908.30C65H40N4S = | 394 | m/z = 704.20C49H28N4S = |
| | 909.12 | | 704.85 |
| 395 | m/z = 704.20C49H28N4S = | 396 | m/z = 802.22C55H35N2OPS = |
| | 704.85 | | 802.93 |
| 397 | m/z = 767.24C55H33N3S = | 398 | m/z = 767.24C55H33N3S = |
| | 767.95 | | 767.95 |
| 399 | m/z = 832.27C59H36N4S = | 400 | m/z = 779.24C56H33N3S = |
| | 833.03 | | 779.96 |
| 401 | m/z = 877.26C62H40NOPS = | 402 | m/z = 842.28C62H38N2S = |
| | 878.04 | | 843.06 |
| 403 | m/z = 830.25C59H34N4S = | 404 | m/z = 777.22C56H31N3S = |
| | 831.01 | | 777.95 |
| 405 | m/z = 875.24C62H38NOPS = | 406 | m/z = 840.26C62H36N2S = |
| | 876.03 | | 841.04 |
| 407 | m/z = 857.25C61H35N3OS = | 408 | m/z. = 873.23C61H35N3S2 = |
| | 858.03 | | 874.09 |
| 409 | m/z = 932.30C67H40N4S = | 410 | m/z = 883.30C64H41N3S = |
| | 933.15 | | 884.11 |
| 411 | m/z = 1007.33C74H45N3S = | 412 | m/z = 1005.32C74H43N3S = |
| | 1008.26 | | 1006.24 |
| 413 | m/z = 705.19C49H27N3OS = | 414 | m/z = 781.22C55H31N3OS = |
| | 705.84 | | 781.93 |
| 415 | m/z = 719.20C50H29N3OS = | 416 | m/z = 721.16C49H27N3S2 = |
| | 719.86 | | 721.90 |
| 417 | m/z = 797.20C55H31N3S2 = | 418 | m/z = 735.18C50H29N3S2 = |
| | 797.99 | | 735.92 |
| 419 | m/z = 780.23C55H32N4S = | 420 | m/z = 856.27C61H36N4S = |
| | 780.95 | | 857.05 |
| 421 | m/z = 794.25C56H34N4S = | 422 | m/z = 731.24C52H33N3S = |
| | 794.98 | | 731.92 |
| 423 | m/z = 807.27C58H37N3S = | 424 | m/z = 745.26C53H35N3S = |
| | 808.02 | | 745.94 |
| 425 | m/z = 855.27C62H37N3S = | 426 | m/z = 931.30C68H41N3S = |
| | 856.06 | | 932.16 |
| 427 | m/z = 869.29C63H39N3S = | 428 | m/z = 853.26C62H35N3S = |
| | 870.09 | | 854.04 |
| 429 | m/z = 929.29C68H39N3S = | 430 | m/z = 867.27C63H37N3S = |
| | 930.14 | | 868.07 |
| 431 | m/z = 652.23C46H28N4O = | 432 | m/z = 728.26C52H32N4O = |
| | 652.76 | | 728.86 |
| 433 | m/z = 883.31C61H37N7O = | 434 | m/z = 882.31C62H38N6O = |
| | 884.02 | | 883.03 |
| 435 | m/z = 882.31C62H38N6O = | 436 | m/z = 881.32C63H39N5O = |
| | 883.03 | | 882.04 |
| 437 | m/z = 881.32C63H39N5O = | 438 | m/z = 881.32C63H39N5O = |
| | 882.04 | | 882.04 |
| 439 | m/z = 727.26C53H33N3O = | 440 | m/z = 727.26C53H33N3O = |
| | 727.87 | | 727.87 |
| 441 | m/z = 743.24C53H33N3S = | 442 | m/z = 727.26C53H33N3O = |
| | 743.93 | | 727.87 |
| 443 | m/z = 741.24C53H31N3O2 = | 444 | m/z = 613.22C44H27N3O = |
| | 741.85 | | 613.72 |
| 445 | m/z = 629.19C44H27N3S = | 446 | m/z = 621.19C43H28NO2P = |
| | 629.78 | | 621.68 |
| 447 | m/z = 637.16C43H28NOPS = | 448 | m/z = 701.25C51H31N3O = |
| | 637.74 | | 701.83 |
| 449 | m/z = 717.22C51H31N3S = | 450 | m/z = 727.26C53H33N3O = |
| | 717.89 | | 727.87 |
| 451 | m/z = 743.24C53H33N3S = | 452 | m/z = 742.24C52H30N4O2 = |
| | 743.93 | | 742.84 |
| 453 | m/z = 832.25C58H32N4O3 = | 454 | m/z = 758.21C52H30N4OS = |
| | 832.92 | | 758.90 |
| 455 | m/z = 848.22C58H32N4O2S = | 456 | m/z = 758.21C52H30N4OS = |
| | 848.98 | | 758.90 |
| 457 | m/z = 864.20C58H32N4OS2 = | 458 | m/z = 774.19C52H30N4S2 = |
| | 865.04 | | 774.96 |
| 459 | m/z = 880.18C58H32N4S3 = | 460 | m/z = 742.24C52H30N4O2 = |
| | 881.10 | | 742.84 |
| 461 | m/z = 832.25C58H32N4O3 = | 462 | m/z = 758.21C52H30N4OS = |
| | 832.92 | | 758.90 |
| 463 | m/z = 848.22C58H32N4O2S = | 464 | m/z = 758.21C52H30N4OS = |
| | 848.98 | | 758.90 |
| 465 | m/z = 880.18C58H32N4S3 = | 466 | m/z = 742.24C52H30N4O2 = |
| | 881.10 | | 742.84 |
| 467 | m/z = 832.25C58H32N4O3 = | 468 | m/z = 758.21C52H30N4OS = |
| | 832.92 | | 758.90 |
| 469 | m/z = 848.22C58H32N4O2S = | 470 | m/z = 758.21C52H30N4OS = |
| | 848.98 | | 758.90 |
| 471 | m/z = 864.20C58H32N4OS2 = | 472 | m/z = 774.19C52H30N4S2 = |
| | 865.04 | | 774.96 |
| 473 | m/z = 880.18C58H32N4S3 = | 474 | m/z = 742.24C52H30N4O2 = |
| | 881.10 | | 742.84 |
| 475 | m/z = 832.25C58H32N4O3 = | 476 | m/z 758.21C52H30N4OS = |
| | 832.92 | | 758.90 |
| 477 | m/z = 848.22C58H32N4O2S = | 478 | m/z = 758.21C52H30N4OS = |
| | 848.98 | | 758.90 |
| 479 | m/z = 864.20C58H32N4OS2 = | 480 | m/z = 774.19C52H30N4S2 = |
| | 865.04 | | 774.96 |
| 481 | m/z = 880.18C58H32N4S3 = | 482 | m/z = 741.24C53H31N3O2 = |
| | 881.10 | | 741.85 |
| 483 | m/z = 831.25C59H33N3O3 = | 484 | m/z = 757.22C53H31N3OS = |
| | 831.93 | | 757.91 |
| 485 | m/z = 847.23C59H33N3O2S = | 486 | m/z = 757.22C53H31N3OS = |
| | 847.99 | | 757.91 |
| 487 | m/z = 863.21C59H33N3OS2 = | 488 | m/z = 773.20C53H31N3S2 = |
| | 864.05 | | 773.97 |
| 489 | m/z = 879.18C59H33N3S3 = | 490 | m/z = 741.24C53H31N3O2 = |
| | 880.11 | | 741.85 |
| 491 | m/z = 831.25C59H33N3O3 = | 492 | m/z = 757.22C53H31N3OS = |
| | 831.93 | | 757.91 |
| 493 | m/z = 847.23C59H33N3O2S = | 494 | m/z = 757.22C53H31N3OS = |
| | 847.99 | | 757.91 |
| 495 | m/z = 879.18C59H33N3S3 = | 496 | m/z = 741.24C53H31N3O2 = |
| | 880.11 | | 741.85 |
| 497 | m/z = 831.25C59H33N3O3 = | 498 | m/z = 757.22C53H31N3OS = |
| | 831.93 | | 757.91 |
| 499 | m/z = 847.23C59H33N3O2S = | 500 | m/z = 757.22C53H31N3OS = |
| | 847.99 | | 757.91 |
| 501 | m/z = 863.21C59H33N3OS2 = | 502 | m/z = 773.20C53H31N3S2 = |
| | 864.05 | | 773.97 |
| 503 | m/z = 879.18C59H33N3S3 = | 504 | m/z = 741.24C53H31N3O2 = |
| | 880.11 | | 741.85 |
| 505 | m/z = 831.25C59H33N3O3 = | 506 | m/z = 757.22C53H31N3OS = |
| | 831.93 | | 757.91 |
| 507 | m/z = 847.23C59H33N3O2S = | 508 | m/z = 757.22C53H31N3OS = |
| | 847.99 | | 757.91 |
| 509 | m/z = 863.21C59H33N3OS2 = | 510 | m/z = 773.20C53H31N3S2 = |
| | 864.05 | | 773.97 |
| 511 | m/z = 879.18C59H33N3S3 = | | |
| | 880.11 | | |

### EXAMPLES

### Example 1

A substrate used for manufacturing a device was ultrasonically cleaned with distilled water for 10 minutes, dried in an oven at 100° C. for 30 minutes, and transferred to a vacuum deposition apparatus chamber.

The substrate used in the present invention was formed in a top emission manner, and an anode electrode was formed as a metal/ITO layer. The metal material used herein may be Ag, Au, Pt, Al, Cu, Ni, Mo, Cr, or an alloy thereof. The indium tin oxide (ITO) may be stacked at a thickness of 7 to 15 nm. On the ITO electrode, a hole injecting layer, a hole transport layer, an electron blocking layer, a light emitting layer, an electron transport layer, and an electron injecting layer are formed sequentially. The hole injecting layer (HIL) was deposited at a thickness of 10 nm and about 3% dopant was added to allow the smooth performance of hole injection. The hole transport layer (HTL) was deposited at a thickness of 120 nm. On the deposited hole transport layer, the electron blocking layer (EBL) was deposited at a thickness of 15 nm. Next, the organic light emitting layer was deposited at a thickness of 20 nm and 5% of dopant was added. Further, on the organic light emitting layer, compound 25 synthesized in Preparation Example 1 and lithium quinolate (LiQ) were formed as the electron transport layer at a weight ratio of 2:1, and deposited at a thickness of 30 nm. During this process, the deposition rate of the organic material was maintained at 0.5 to 1.0 Å/sec, and the vacuum degree at the time of deposition was maintained at 1 to 4×10⁻⁷ torr. To form a resonance structure, the total thickness of the organic material has a specific thickness according to the luminescent color. Further, in order to maximize the resonance effect, the electrode was constituted as a semi-transparent electrode (cathode). The metal used for this electrode may include Al, Mg, Ag, LiF, or an alloy thereof, and the ratio and specific thickness are applied so that a light reflection characteristic is generated. The thickness of the negative electrode used was 14 nm. Finally, a light efficiency improvement layer (capping layer) was deposited at a thickness of 63 nm. After vacuum deposition, the substrate was transferred to a glove box, and a sealing process was performed. The sealing member may be a glass cap provided with a moisture absorbent (getter) therein, and a sealing resin material may be applied to perform UV curing and to block permeation of oxygen and moisture into the deposition surface.

### Example 2

Example 2 was prepared in the same manner as in Example 1, except that Compound 26 was used instead of Compound 25 as the electron transport layer.

### Example 3

Example 3 was prepared in the same manner as in Example 1, except that Compound 28 was used instead of Compound 25 as the electron transport layer.

### Example 4

Example 4 was prepared in the same manner as in Example 1, except that Compound 29 was used instead of Compound 25 as the electron transport layer.

### Example 5

Example 5 was prepared in the same manner as in Example 1, except that Compound 31 was used instead of Compound 25 as the electron transport layer.

### Example 6

Example 6 was prepared in the same manner as in Example 1, except that Compound 35 was used instead of Compound 25 as the electron transport layer.

### Example 7

Example 7 was prepared in the same manner as in Example 1, except that Compound 168 was used instead of Compound 25 as the electron transport layer.

### Example 8

Example 8 was prepared in the same manner as in Example 1, except that Compound 169 was used instead of Compound 25 as the electron transport layer.

### Example 9

Example 9 was prepared in the same manner as in Example 1, except that Compound 171 was used instead of Compound 25 as the electron transport layer.

### Example 10

Example 10 was prepared in the same manner as in Example 1, except that Compound 172 was used instead of Compound 25 as the electron transport layer.

### Example 11

Example 11 was prepared in the same manner as in Example 1, except that Compound 174 was used instead of Compound 25 as the electron transport layer.

### Example 12

Example 12 was prepared in the same manner as in Example 1, except that Compound 178 was used instead of Compound 25 as the electron transport layer.

### Example 13

Example 13 was prepared in the same manner as in Example 1, except that Compound 448 was used instead of Compound 25 as the electron transport layer.

### Example 14

Example 14 was prepared in the same manner as in Example 1, except that Compound 450 was used instead of Compound 25 as the electron transport layer.

### Example 14

Example 15 was prepared in the same manner as in Example 1, except that Compound 452 was used instead of Compound 25 as the electron transport layer.

### Comparative Example 1

Comparative Example 1 was prepared in the same manner as in Example 1, except that Compound ET1 was used instead of Compound 25 as the electron transport layer.

### Comparative Example 2

Comparative Example 2 was prepared in the same manner as in Example 1, except that Compound ET2 was used instead of Compound 25 as the electron transport layer.

### Comparative Example 3

Comparative Example 3 was prepared in the same manner as in Example 1, except that Compound ET3 was used instead of Compound 25 as the electron transport layer.

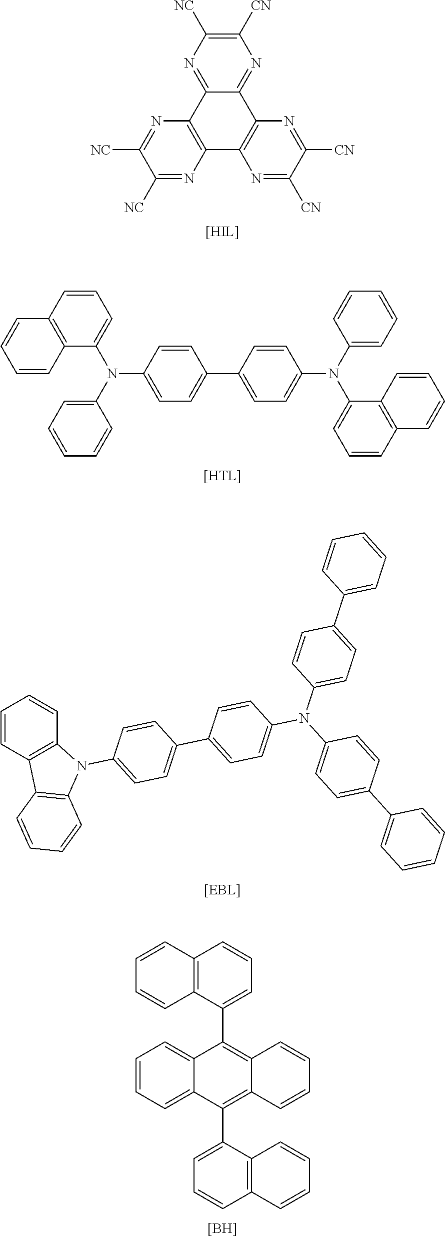

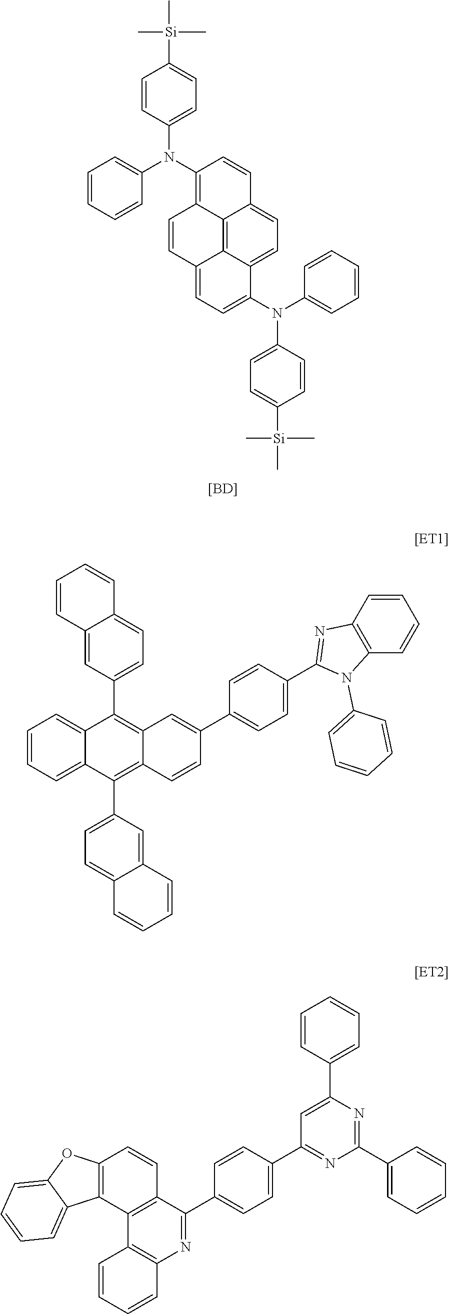

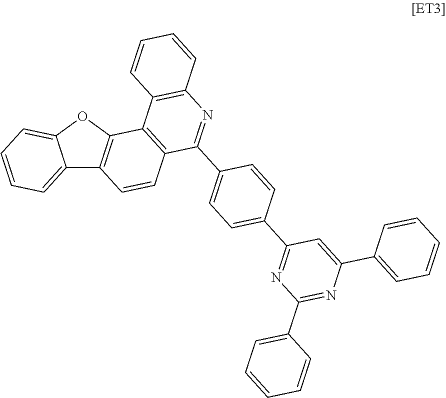 [SMILES: c1ccc(-c2cc(-c3ccc(-c4nc5ccccc5c5c4ccc4c6ccccc6oc45)cc3)nc(-c3ccccc3)n2)cc1]

The driving voltages and light emitting efficiencies of the organic light emitting devices were measured at a current density of 10 mA/cm², and the time (LT95) corresponding to 95% relative to the initial luminance of 1,000 cd/m² was also measured. The results of the measurements are listed in Table 5 below.

TABLE 5

| | | | Current | |
|---|---|---|---|---|
| | | Voltage | Efficiency | Life Time 95 at |
| | Compound | (V) | (cd/A) | 1000 cd/m² |
| Example 1 | 25 | 4.31 | 7.43 | 112 |
| Example 2 | 26 | 4.03 | 7.58 | 189 |
| Example 3 | 28 | 4.06 | 7.51 | 182 |
| Example 4 | 29 | 4.10 | 7.23 | 191 |
| Example 5 | 31 | 4.21 | 7.09 | 113 |
| Example 6 | 35 | 4.06 | 7.52 | 120 |
| Example 7 | 168 | 4.12 | 7.38 | 136 |
| Example 8 | 169 | 4.07 | 7.55 | 159 |
| Example 9 | 171 | 4.19 | 7.53 | 181 |
| Example 10 | 172 | 4.23 | 7.29 | 121 |
| Example 11 | 174 | 4.13 | 7.26 | 128 |
| Example 12 | 178 | 4.09 | 7.49 | 133 |
| Example 13 | 448 | 4.10 | 7.43 | 136 |
| Example 14 | 450 | 4.12 | 7.40 | 139 |
| Example 15 | 452 | 4.19 | 7.33 | 131 |
| Comparative | ET1 | 4.58 | 6.71 | 105 |
| Example 1 | | | | |
| Comparative | ET2 | 4.37 | 6.91 | 110 |
| Example 2 | | | | |
| Comparative | ET3 | 4.41 | 6.84 | 101 |
| Example 3 | | | | |

Referring to Table 5 above, it was confirmed that the light emitting devices according to Examples 1 to 15 have higher efficiencies, longer lifetimes, and lower driving voltages than those of Comparative Examples 1 to 3.

The compounds according to the embodiments of the present invention can be employed as a hole injecting material, a hole transporting material, a host material, a hole blocking material, an electron injecting material, an electron transporting material, or a charge generating material of an organic light emitting device. In particular, the compounds according to the embodiments of the present invention can be effectively employed as an electron injecting material or an electron transporting material, a hole blocking material, an n-type charge generating material, a p-type or n-type phosphorescent green (G) host material, or a p-type or n-type phosphorescent yellow green (YG) host material. The organic light emitting device using the compounds according to the embodiments of the present invention can have excellent electrochemical and thermal stability, thus resulting in achievement of excellent lifetime characteristics and high light emitting efficiency, even at a low driving voltage.

In addition, it is possible to manufacture an organic light emitting device with high efficiency, long lifetime, high color purity, and low driving voltage using the compound represented by Chemical Formula 1 of the present invention.

Further, the compound represented by Chemical Formula 1 of the present invention has an enhanced hole blocking function due to a low highest occupied molecular orbital (HOMO) energy level, thereby resulting in the achievement of high efficiency and long lifetime characteristics.

What is claimed is:

1. A compound represented by Chemical Formula 1 below: 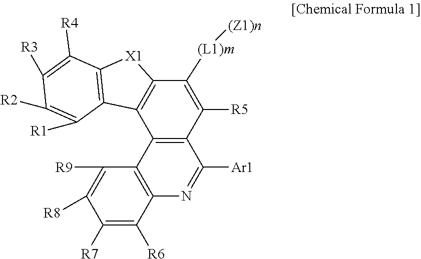
In Chemical Formula 1,
X1 is S or O,
L1 is a direct bond; a substituted or unsubstituted arylene group; or a substituted or unsubstituted heteroarylene group,
Z1 is hydrogen; a substituted or unsubstituted N-containing heterocyclic group; a substituted or unsubstituted amine group; or —P(═O)RaRb,
m is an integer of 0 to 4, and each L1 is the same as or different from each other when m is 2 or higher,
n is an integer of 1 to 4, and each Z1 is the same as or different from each other when n is 2 or higher,
Ar1 is a substituted or unsubstituted aryl group; or a substituted or unsubstituted heteroaryl group,
R1 to R9, Ra, and Rb are the same as or different from each other, and each, independently, selected from the group consisting of hydrogen; deuterium; a halogen group; —CN; a substituted or unsubstituted alkyl group; a substituted or unsubstituted alkenyl group; a substituted or unsubstituted alkynyl group; a substituted or unsubstituted alkoxy group; a substituted or unsubstituted cycloalkyl group; a substituted or unsubstituted heterocycloalkyl group; a substituted or unsubstituted aryl group; a substituted or unsubstituted heteroaryl group; —SiRR′R″; —P(═O)RR′; and an amine group substituted or unsubstituted with an alkyl group, an aryl group, or a heteroaryl group, and R, R′, and R″ are the same as or different from each other, and each, independently, hydrogen; deuterium; —CN; a substituted or unsubstituted alkyl group; a substituted or unsubstituted cycloalkyl group; a substituted or unsubstituted aryl group;
or a substituted or unsubstituted heteroaryl group.

2. The compound of claim 1, wherein
the Z1 of Chemical Formula 1 is hydrogen; or one of Chemical Formulas 2 to 9 below: 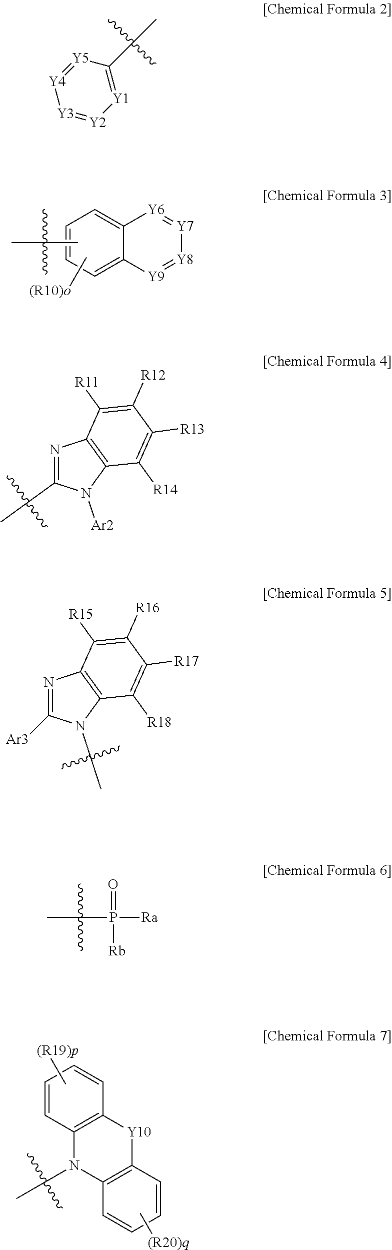 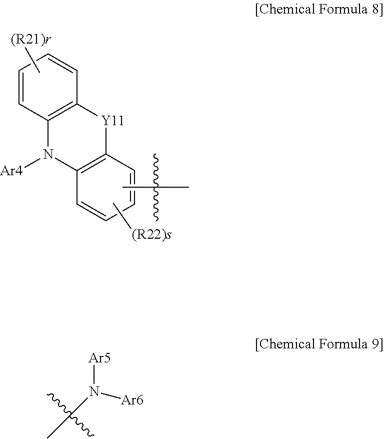
In Chemical Formulas 2 to 9, Y1 to Y9 are the same as or different from each other and each, independently, N or CRc; at least one of Y1 to Y5 is N; at least one of Y6 to Y9 is N; Y10 and Y11 are the same as or different from each other and each, independently, a direct bond, O, S, or CRdRe; Ar2 to Ar6, R10 to R22, and Rc to Re are the same as or different from each other and each, independently, selected from the group consisting of hydrogen, deuterium, a substituted or unsubstituted alkyl group, a substituted or unsubstituted aryl group, and a substituted or unsubstituted heteroaryl group; two adjacent groups among these groups may be bonded to each other to form a substituted or unsubstituted aromatic hydrocarbon ring or a substituted or unsubstituted hetero ring; o is an integer of 1 to 3, and each R10 is the same as or different from each other when o is 2 or higher; p is an integer of 1 to 4, and each R19 is the same as or different from each other when p is 2 or higher; q is an integer of 1 to 4, and each R20 is the same as or different from each other when q is 2 or higher; r is an integer of 1 to 4, and each R21 is the same as or different from each other when r is 2 or higher; s is an integer of 1 to 3, and each R22 are the same as or different from each other when s is 2 or higher; and the definition of the rest of the substituents are the same as Chemical Formula 1.

3. The compound of claim 2, wherein
Chemical Formula 2 is selected from the structural formulas below: 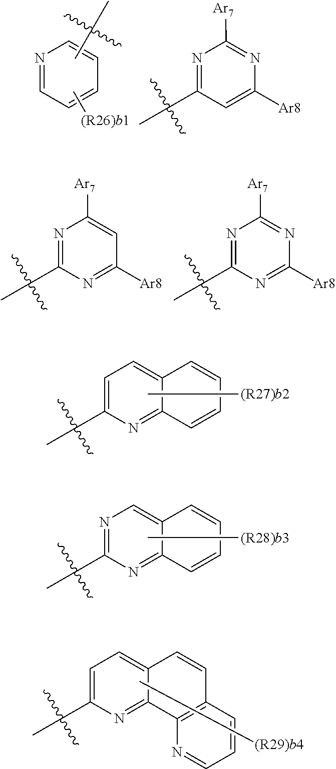
R26 to R29, Ar7, and Ar8 are the same as being defined for the substituent Rc in Chemical Formula 1; b1 is an integer of 1 to 4, and each R26 is the same as or different from each other when b1 is 2 or higher; b2 is an integer of 1 to 6, and each R27 is the same as or different from each other when b2 is 2 or higher; b3 is an integer of 1 to 5, and each R28 are the same as or different from each other when b3 is 2 or higher; and b4 is an integer of 1 to 7, and each R29 are the same as or different from each other when b4 is 2 or higher.

4. The compound of claim 2, wherein
Chemical Formula 3 is selected from the structural formulas below: 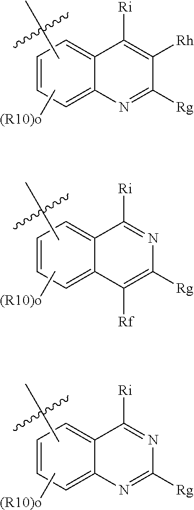
In the above structural formulas, Rf to Ri are the same as for the definition provided for the substituent Rc in Chemical Formula 2, and o and R10 are the same as being defined in Chemical Formula 3.

5. The compound of claim 1, wherein
Ar1 may be represented by any one of a C6-C20 aryl group; or the structural formulas below: 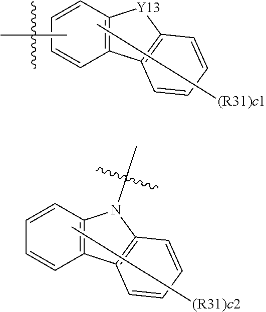
In the structural formulas above, Y13 is O, S, CRjRk or NRm; R31, R32, Rj, Rk, and Rm are the same as or different from each other, and each, independently, selected from the group consisting of hydrogen, deuterium, a substituted or unsubstituted alkyl group, a substituted or unsubstituted aryl group, and a substituted or unsubstituted heteroaryl group; adjacent substituents may be bonded to each other to form a hydrocarbon ring or a heterocyclic group; cl is an integer of 1 to 7, and each R31 is the same as or different from each other when c1 is 2 or higher; and c2 is an integer of 1 to 8, and each R32 is the same as or different from each other when c2 is 2 or higher.

6. The compound of claim 1, wherein
Chemical Formula 1 is selected from the structural formulas below:

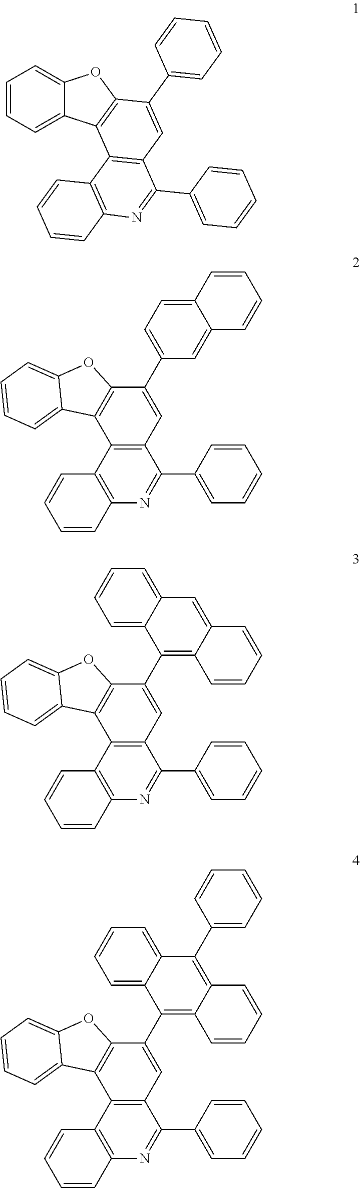

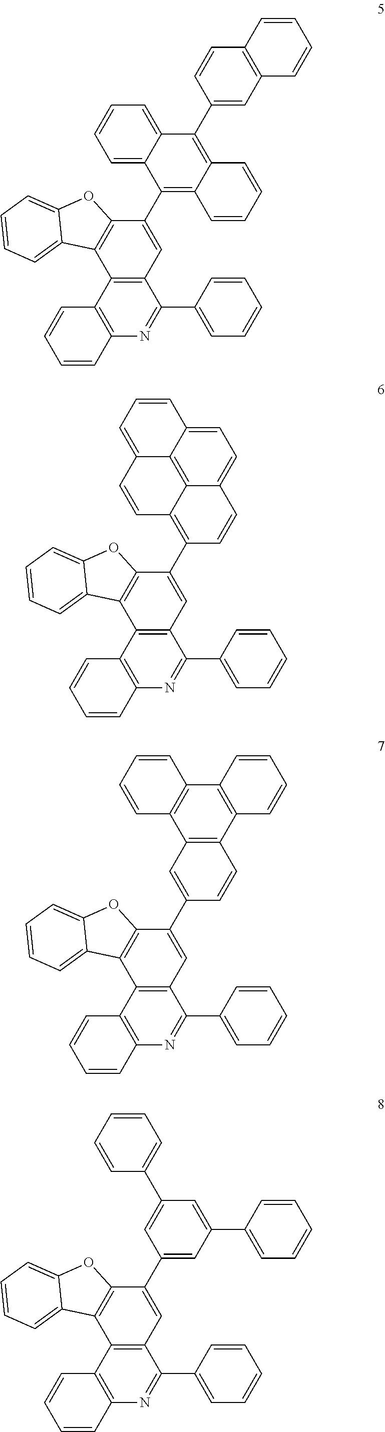

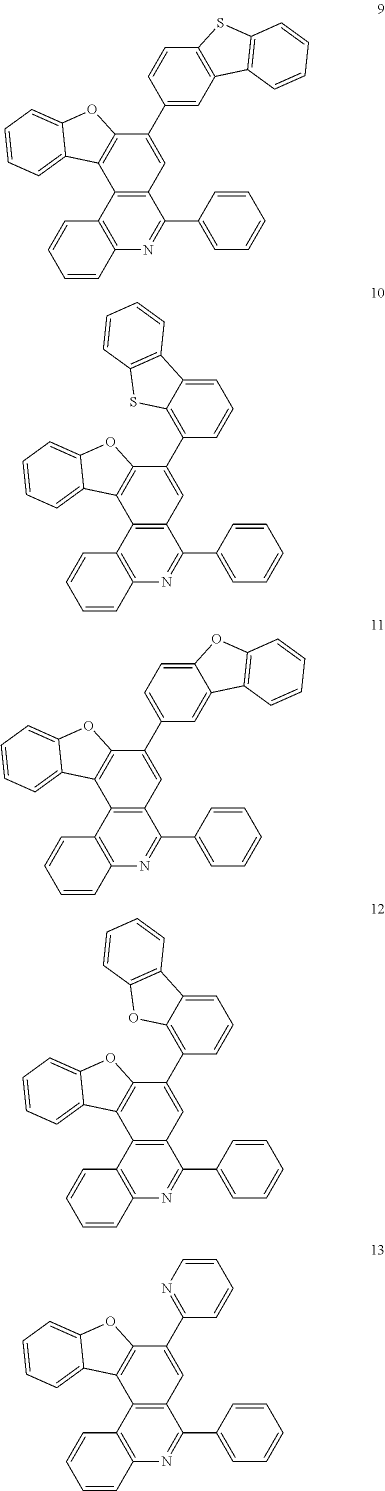

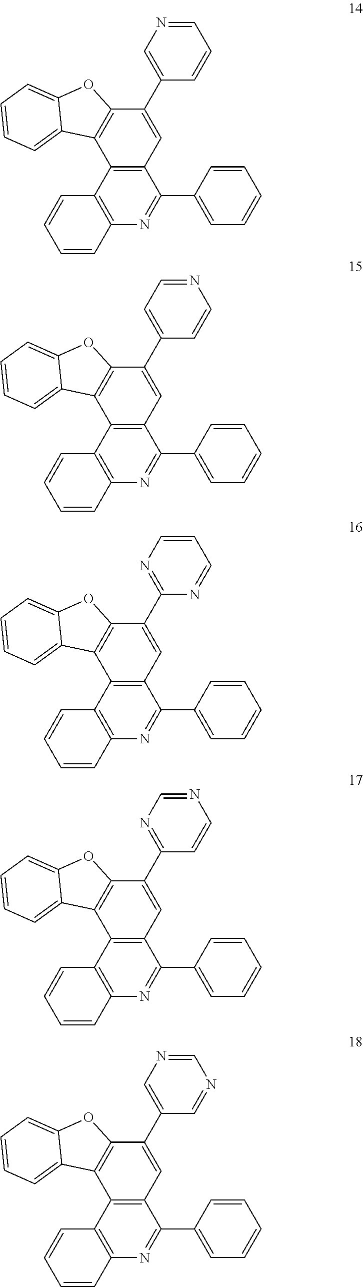 [SMILES: c1ccc(-c2nc3ccccc3c3c2cc(-c2cccnc2)c2oc4ccccc4c23)cc1 ; c1ccc(-c2nc3ccccc3c3c2cc(-c2ccncc2)c2oc4ccccc4c23)cc1 ; c1ccc(-c2nc3ccccc3c3c2cc(-c2ncccn2)c2oc4ccccc4c23)cc1 ; c1ccc(-c2nc3ccccc3c3c2cc(-c2ccncn2)c2oc4ccccc4c23)cc1 ; c1ccc(-c2nc3ccccc3c3c2cc(-c2cncnc2)c2oc4ccccc4c23)cc1]

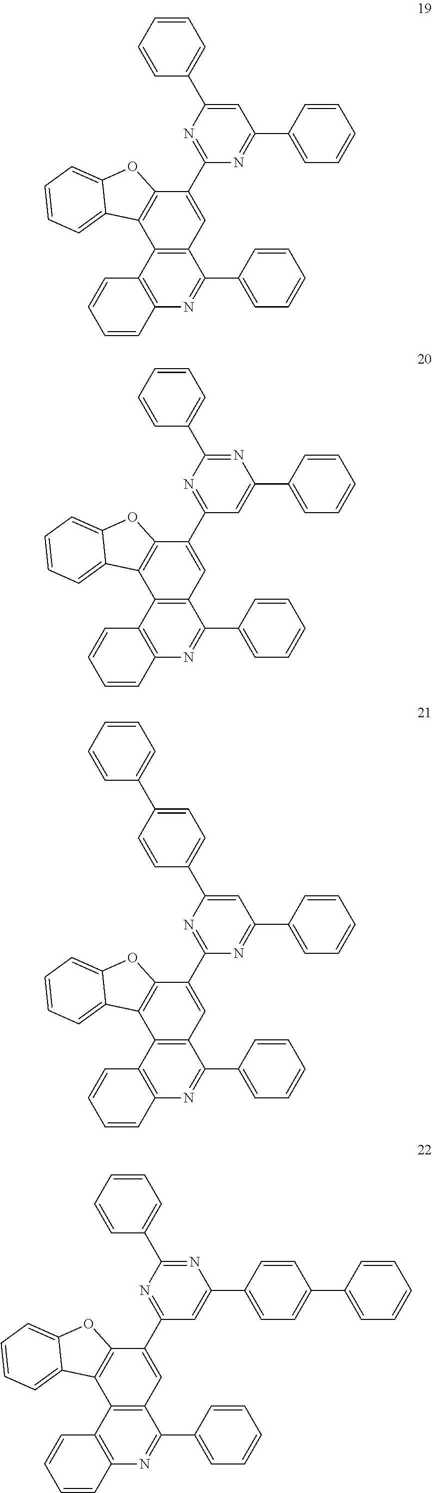

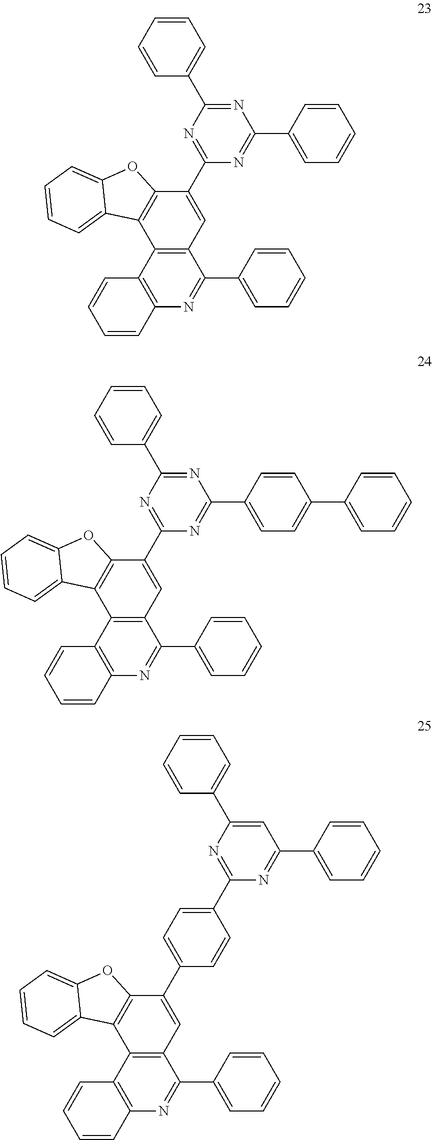 [SMILES: c1ccc(-c2nc(-c3ccccc3)nc(-c3cc4c(-c5ccccc5)nc5ccccc5c4c4c3oc3ccccc34)n2)cc1 ; c1ccc(-c2ccc(-c3nc(-c4ccccc4)nc(-c4cc5c(-c6ccccc6)nc6ccccc6c5c5c4oc4ccccc45)n3)cc2)cc1 ; c1ccc(-c2cc(-c3ccccc3)nc(-c3ccc(-c4cc5c(-c6ccccc6)nc6ccccc6c5c5c4oc4ccccc45)cc3)n2)cc1]

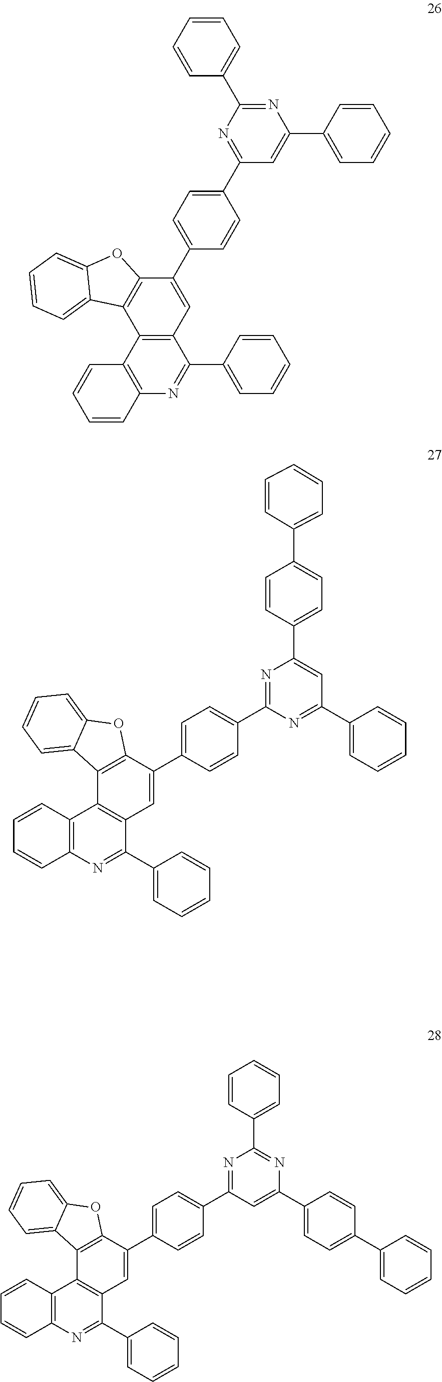 [SMILES: c1ccc(-c2cc(-c3ccc(-c4cc5c(-c6ccccc6)nc6ccccc6c5c5c4oc4ccccc45)cc3)nc(-c3ccccc3)n2)cc1 ; c1ccc(-c2ccc(-c3cc(-c4ccccc4)nc(-c4ccc(-c5cc6c(-c7ccccc7)nc7ccccc7c6c6c5oc5ccccc56)cc4)n3)cc2)cc1 ; c1ccc(-c2ccc(-c3cc(-c4ccc(-c5cc6c(-c7ccccc7)nc7ccccc7c6c6c5oc5ccccc56)cc4)nc(-c4ccccc4)n3)cc2)cc1]

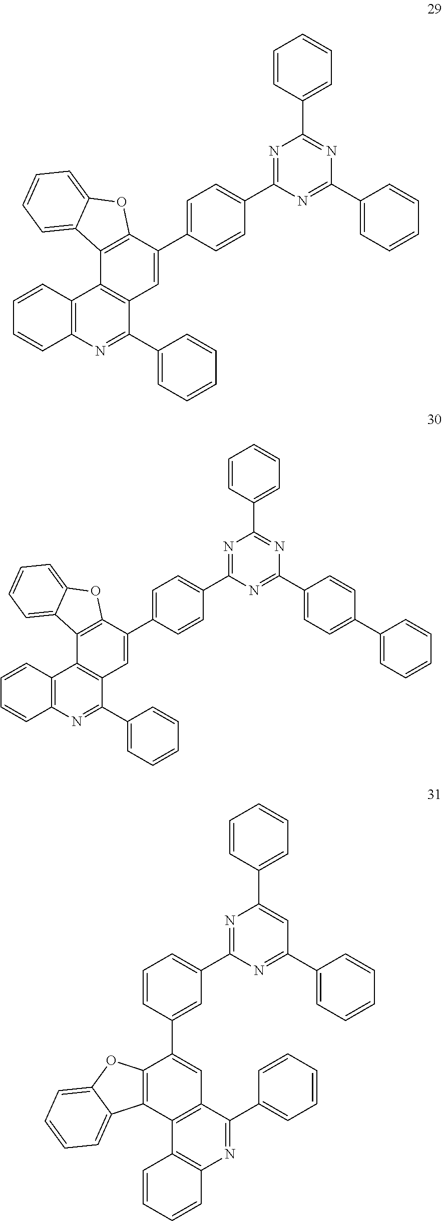 [SMILES: c1ccc(-c2nc(-c3ccccc3)nc(-c3ccc(-c4cc5c(-c6ccccc6)nc6ccccc6c5c5c4oc4ccccc45)cc3)n2)cc1 ; c1ccc(-c2ccc(-c3nc(-c4ccccc4)nc(-c4ccc(-c5cc6c(-c7ccccc7)nc7ccccc7c6c6c5oc5ccccc56)cc4)n3)cc2)cc1 ; c1ccc(-c2cc(-c3ccccc3)nc(-c3cccc(-c4cc5c(-c6ccccc6)nc6ccccc6c5c5c4oc4ccccc45)c3)n2)cc1]

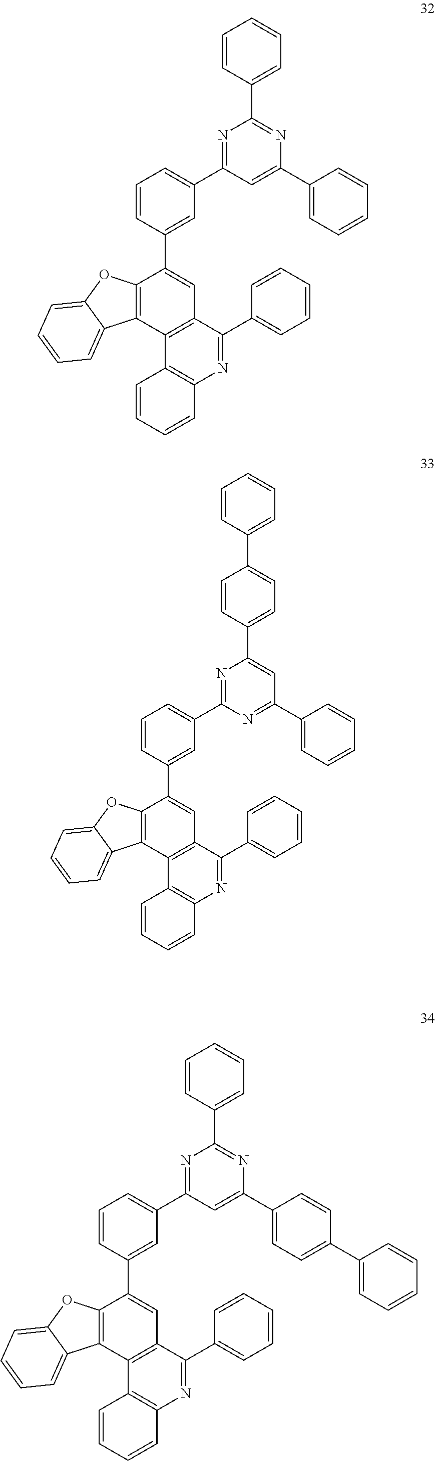 [SMILES: c1ccc(-c2cc(-c3cccc(-c4cc5c(-c6ccccc6)nc6ccccc6c5c5c4oc4ccccc45)c3)nc(-c3ccccc3)n2)cc1 ; c1ccc(-c2ccc(-c3cc(-c4ccccc4)nc(-c4cccc(-c5cc6c(-c7ccccc7)nc7ccccc7c6c6c5oc5ccccc56)c4)n3)cc2)cc1 ; c1ccc(-c2ccc(-c3cc(-c4cccc(-c5cc6c(-c7ccccc7)nc7ccccc7c6c6c5oc5ccccc56)c4)nc(-c4ccccc4)n3)cc2)cc1]

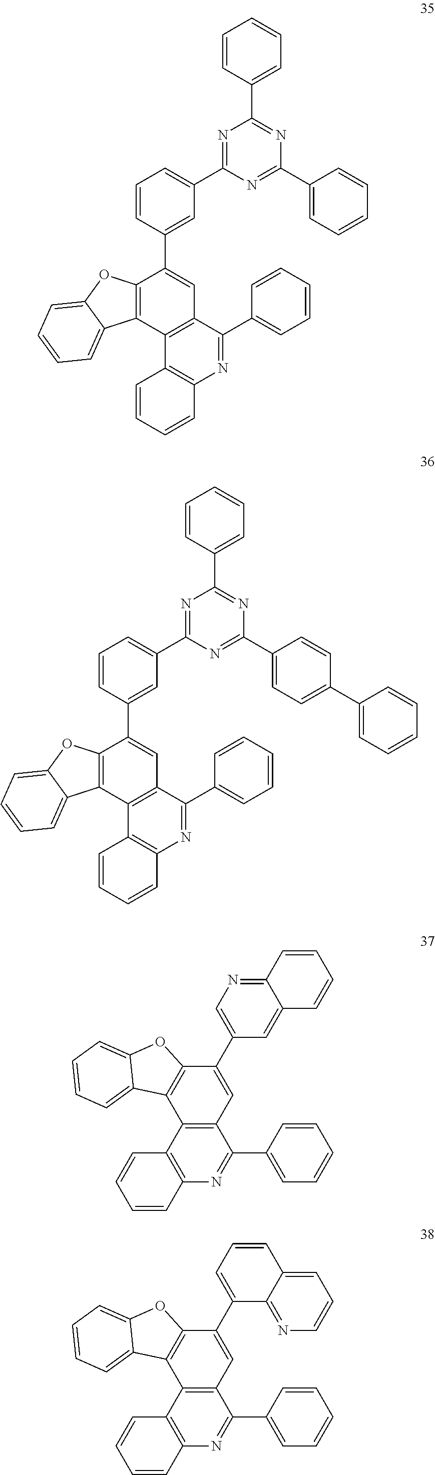 [SMILES: c1ccc(-c2nc(-c3ccccc3)nc(-c3cccc(-c4cc5c(-c6ccccc6)nc6ccccc6c5c5c4oc4ccccc45)c3)n2)cc1 ; c1ccc(-c2ccc(-c3nc(-c4ccccc4)nc(-c4cccc(-c5cc6c(-c7ccccc7)nc7ccccc7c6c6c5oc5ccccc56)c4)n3)cc2)cc1 ; c1ccc(-c2nc3ccccc3c3c2cc(-c2cnc4ccccc4c2)c2oc4ccccc4c23)cc1 ; c1ccc(-c2nc3ccccc3c3c2cc(-c2cccc4cccnc24)c2oc4ccccc4c23)cc1]

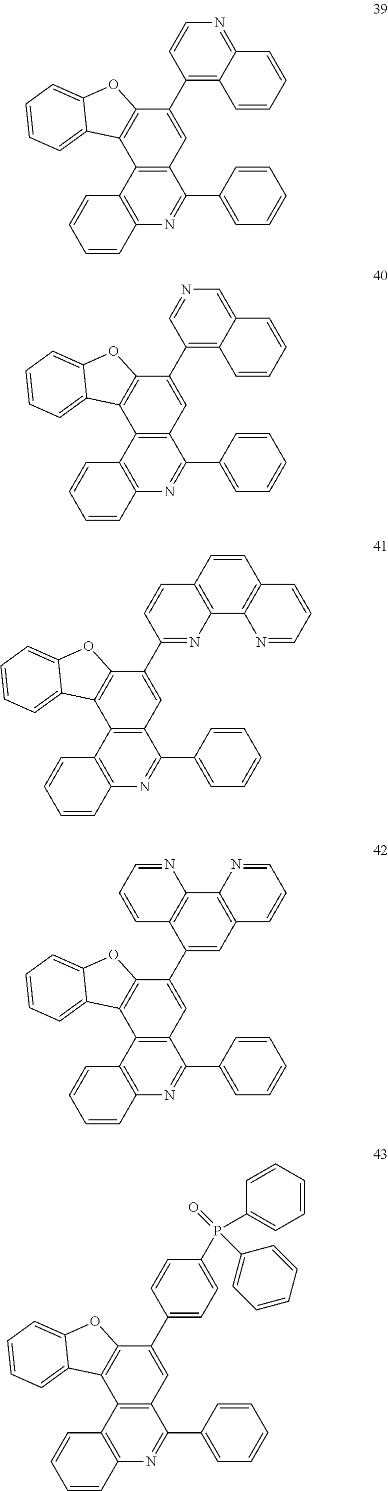 [SMILES: c1ccc(-c2nc3ccccc3c3c2cc(-c2ccnc4ccccc24)c2oc4ccccc4c23)cc1 ; c1ccc(-c2nc3ccccc3c3c2cc(-c2cncc4ccccc24)c2oc4ccccc4c23)cc1 ; c1ccc(-c2nc3ccccc3c3c2cc(-c2ccc4ccc5cccnc5c4n2)c2oc4ccccc4c23)cc1 ; c1ccc(-c2nc3ccccc3c3c2cc(-c2cc4cccnc4c4ncccc24)c2oc4ccccc4c23)cc1 ; O=P(c1ccccc1)(c1ccccc1)c1ccc(-c2cc3c(-c4ccccc4)nc4ccccc4c3c3c2oc2ccccc23)cc1]

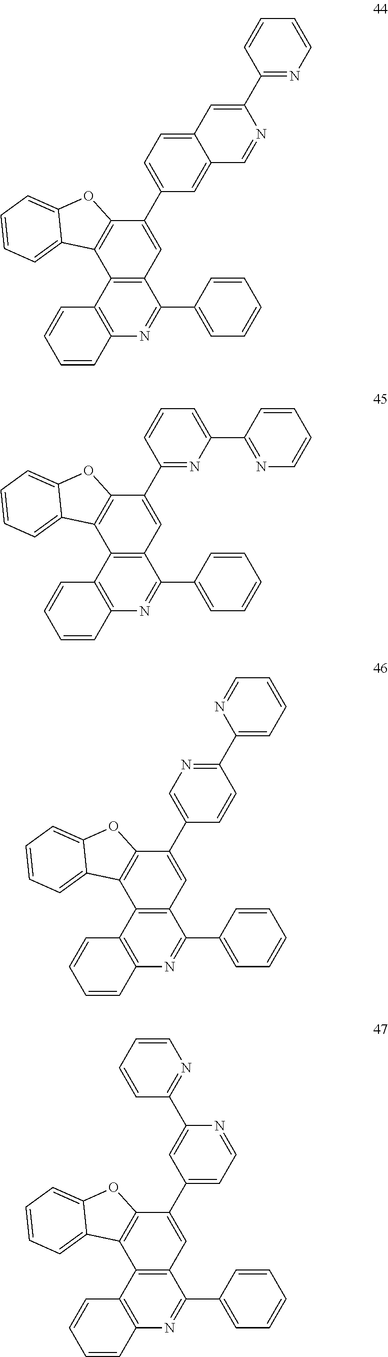

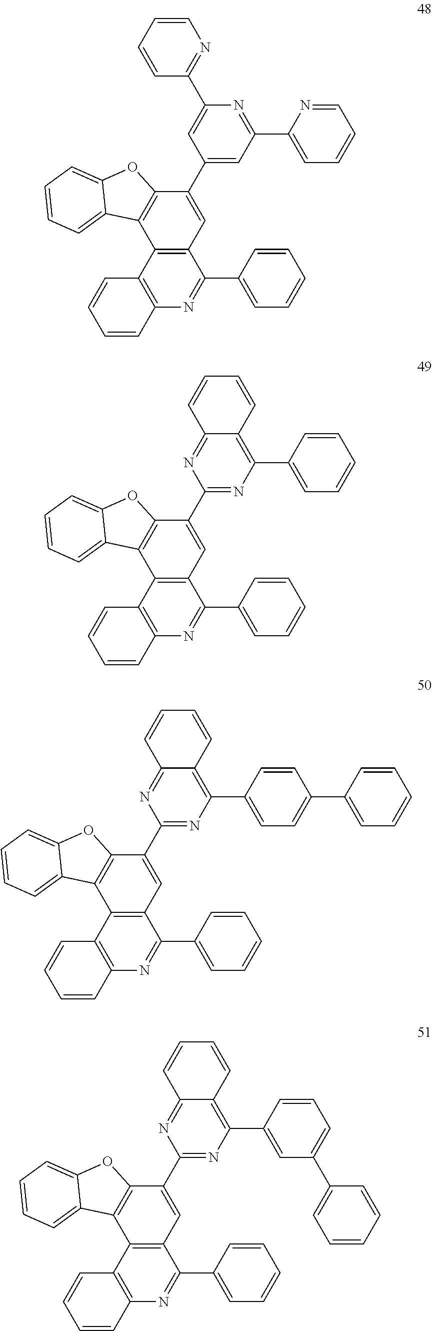 [SMILES: c1ccc(-c2nc3ccccc3c3c2cc(-c2cc(-c4ccccn4)nc(-c4ccccn4)c2)c2oc4ccccc4c23)cc1 ; c1ccc(-c2nc(-c3cc4c(-c5ccccc5)nc5ccccc5c4c4c3oc3ccccc34)nc3ccccc23)cc1 ; c1ccc(-c2ccc(-c3nc(-c4cc5c(-c6ccccc6)nc6ccccc6c5c5c4oc4ccccc45)nc4ccccc34)cc2)cc1 ; c1ccc(-c2cccc(-c3nc(-c4cc5c(-c6ccccc6)nc6ccccc6c5c5c4oc4ccccc45)nc4ccccc34)c2)cc1]

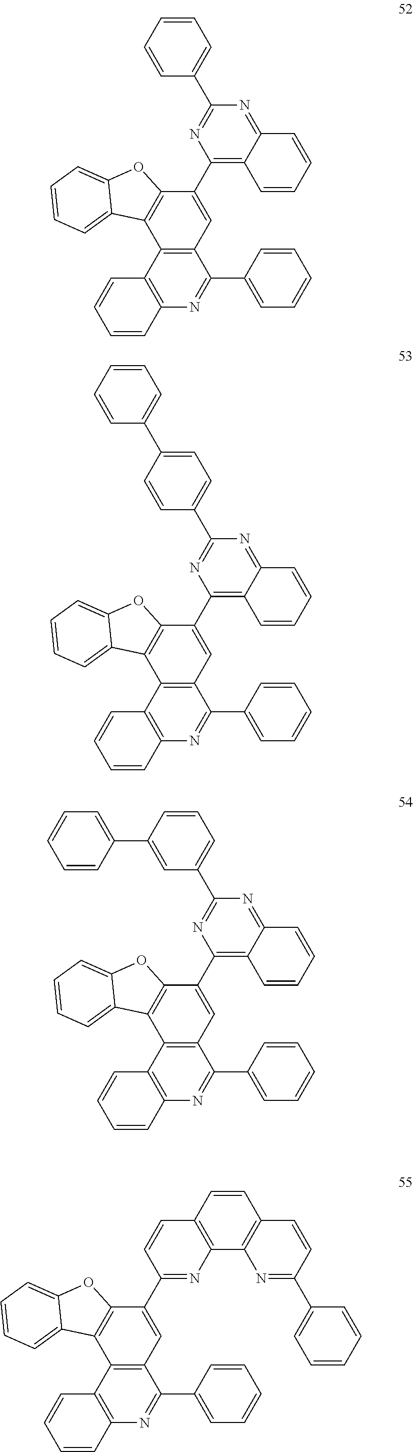

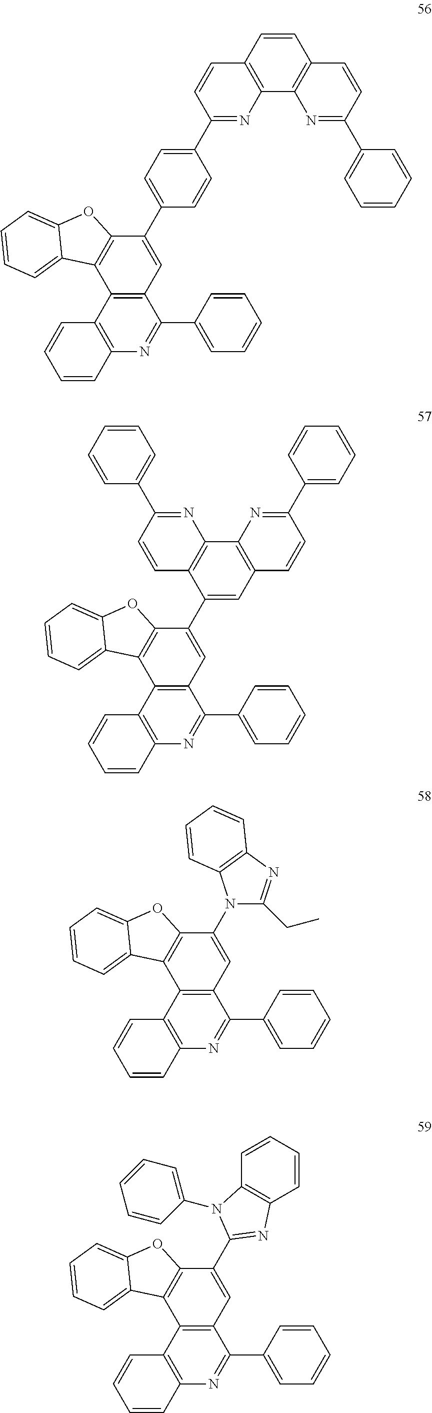 [SMILES: c1ccc(-c2ccc3ccc4ccc(-c5ccc(-c6cc7c(-c8ccccc8)nc8ccccc8c7c7c6oc6ccccc67)cc5)nc4c3n2)cc1 ; c1ccc(-c2ccc3cc(-c4cc5c(-c6ccccc6)nc6ccccc6c5c5c4oc4ccccc45)c4ccc(-c5ccccc5)nc4c3n2)cc1 ; CCc1nc2ccccc2n1-c1cc2c(-c3ccccc3)nc3ccccc3c2c2c1oc1ccccc12 ; c1ccc(-c2nc3ccccc3c3c2cc(-c2nc4ccccc4n2-c2ccccc2)c2oc4ccccc4c23)cc1]

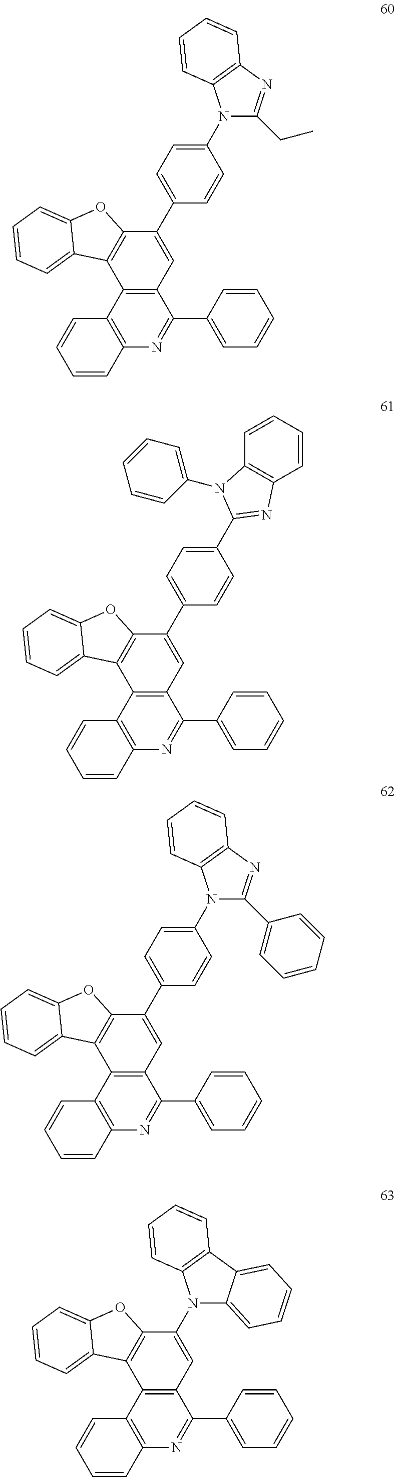 [SMILES: CCc1nc2ccccc2n1-c1ccc(-c2cc3c(-c4ccccc4)nc4ccccc4c3c3c2oc2ccccc23)cc1 ; c1ccc(-c2nc3ccccc3c3c2cc(-c2ccc(-c4nc5ccccc5n4-c4ccccc4)cc2)c2oc4ccccc4c23)cc1 ; c1ccc(-c2nc3ccccc3c3c2cc(-c2ccc(-n4c(-c5ccccc5)nc5ccccc54)cc2)c2oc4ccccc4c23)cc1 ; c1ccc(-c2nc3ccccc3c3c2cc(-n2c4ccccc4c4ccccc42)c2oc4ccccc4c23)cc1]

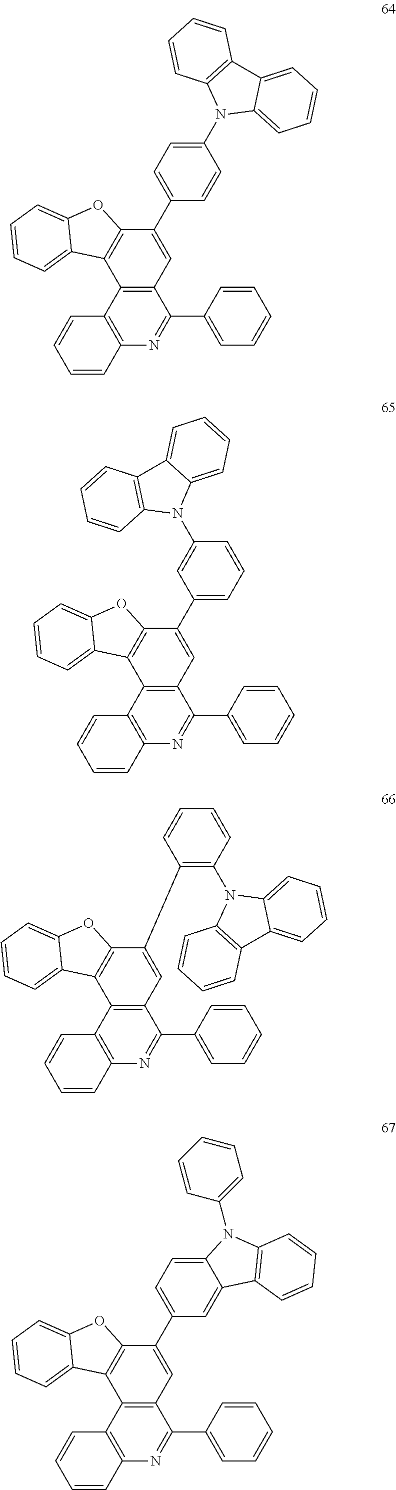 [SMILES: c1ccc(-c2nc3ccccc3c3c2cc(-c2ccc(-n4c5ccccc5c5ccccc54)cc2)c2oc4ccccc4c23)cc1 ; c1ccc(-c2nc3ccccc3c3c2cc(-c2cccc(-n4c5ccccc5c5ccccc54)c2)c2oc4ccccc4c23)cc1 ; c1ccc(-c2nc3ccccc3c3c2cc(-c2ccccc2-n2c4ccccc4c4ccccc42)c2oc4ccccc4c23)cc1 ; c1ccc(-c2nc3ccccc3c3c2cc(-c2ccc4c(c2)c2ccccc2n4-c2ccccc2)c2oc4ccccc4c23)cc1]

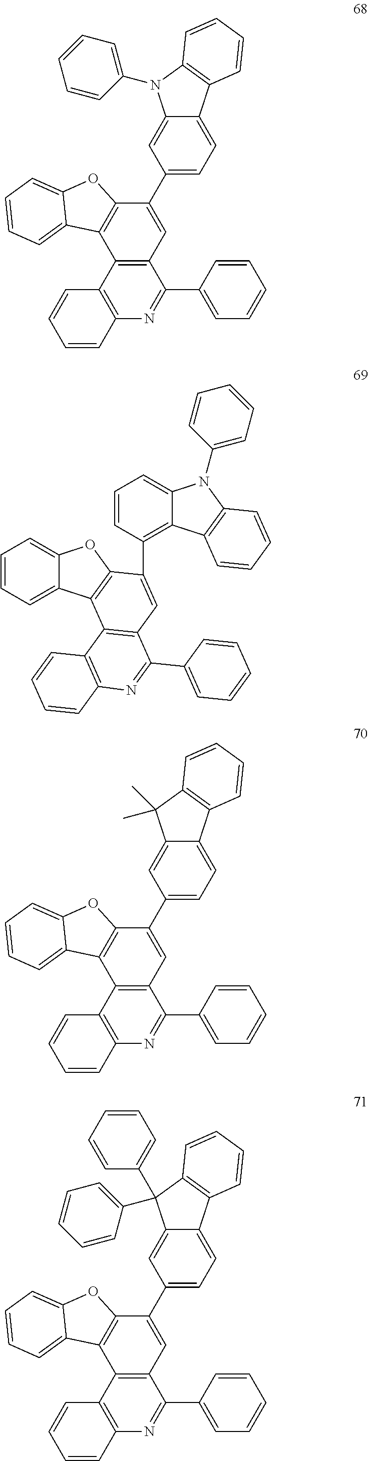

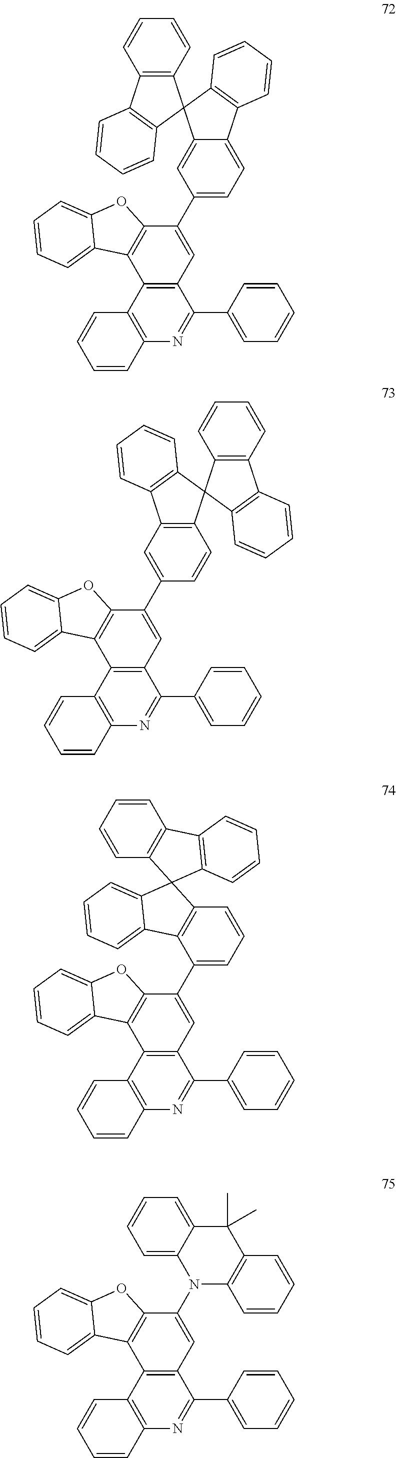

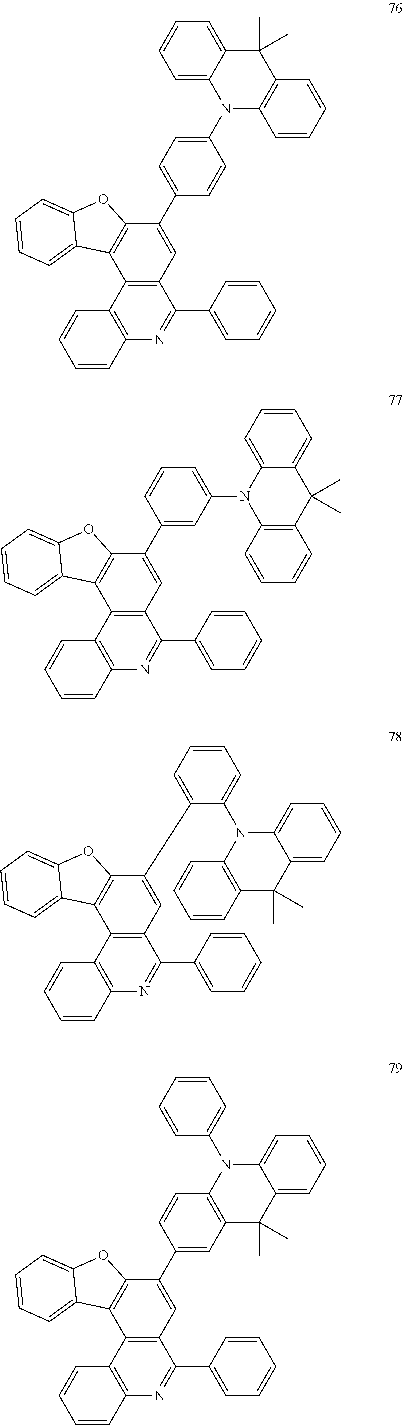 [SMILES: CC1(C)c2ccccc2N(c2ccc(-c3cc4c(-c5ccccc5)nc5ccccc5c4c4c3oc3ccccc34)cc2)c2ccccc21 ; CC1(C)c2ccccc2N(c2cccc(-c3cc4c(-c5ccccc5)nc5ccccc5c4c4c3oc3ccccc34)c2)c2ccccc21 ; CC1(C)c2ccccc2N(c2ccccc2-c2cc3c(-c4ccccc4)nc4ccccc4c3c3c2oc2ccccc23)c2ccccc21 ; CC1(C)c2ccccc2N(c2ccccc2)c2ccc(-c3cc4c(-c5ccccc5)nc5ccccc5c4c4c3oc3ccccc34)cc21]

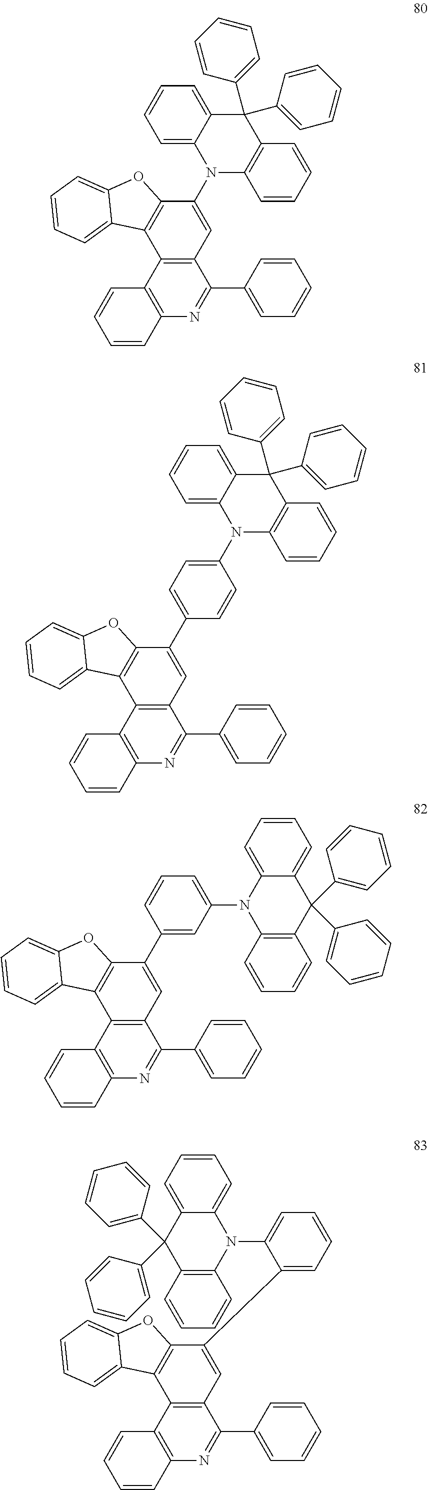 [SMILES: c1ccc(-c2nc3ccccc3c3c2cc(N2c4ccccc4C(c4ccccc4)(c4ccccc4)c4ccccc42)c2oc4ccccc4c23)cc1 ; c1ccc(-c2nc3ccccc3c3c2cc(-c2ccc(N4c5ccccc5C(c5ccccc5)(c5ccccc5)c5ccccc54)cc2)c2oc4ccccc4c23)cc1 ; c1ccc(-c2nc3ccccc3c3c2cc(-c2cccc(N4c5ccccc5C(c5ccccc5)(c5ccccc5)c5ccccc54)c2)c2oc4ccccc4c23)cc1 ; c1ccc(-c2nc3ccccc3c3c2cc(-c2ccccc2N2c4ccccc4C(c4ccccc4)(c4ccccc4)c4ccccc42)c2oc4ccccc4c23)cc1]

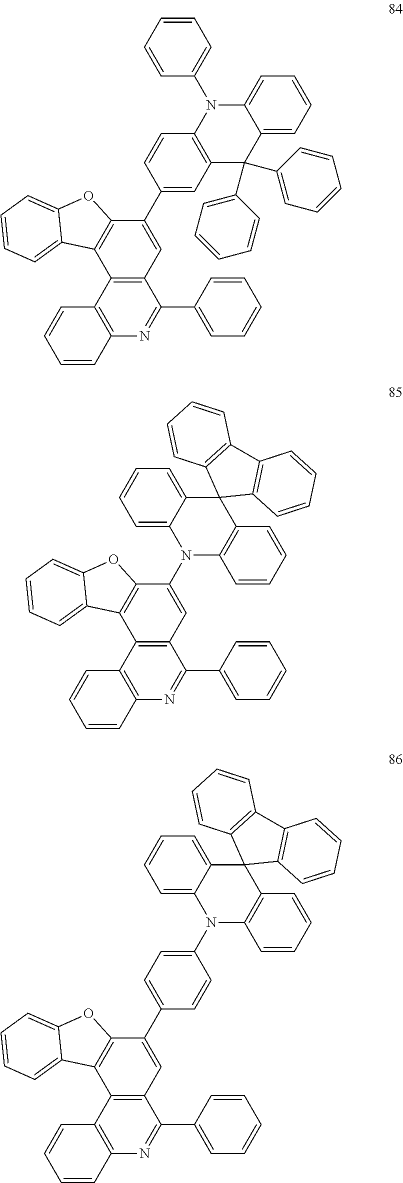 [SMILES: c1ccc(-c2nc3ccccc3c3c2cc(-c2ccc4c(c2)C(c2ccccc2)(c2ccccc2)c2ccccc2N4c2ccccc2)c2oc4ccccc4c23)cc1 ; c1ccc(-c2nc3ccccc3c3c2cc(N2c4ccccc4C4(c5ccccc5-c5ccccc54)c4ccccc42)c2oc4ccccc4c23)cc1 ; c1ccc(-c2nc3ccccc3c3c2cc(-c2ccc(N4c5ccccc5C5(c6ccccc6-c6ccccc65)c5ccccc54)cc2)c2oc4ccccc4c23)cc1]

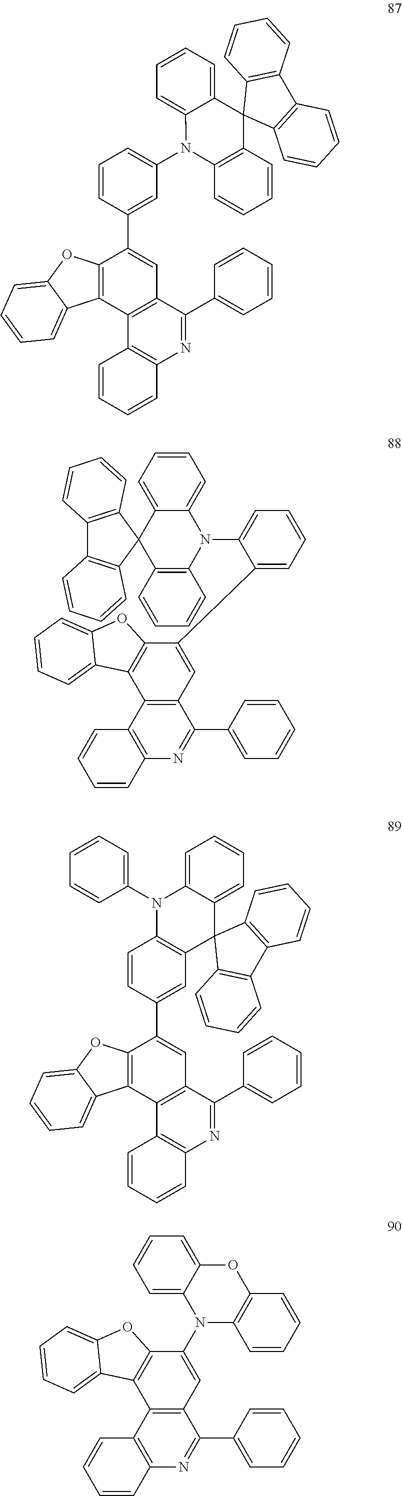

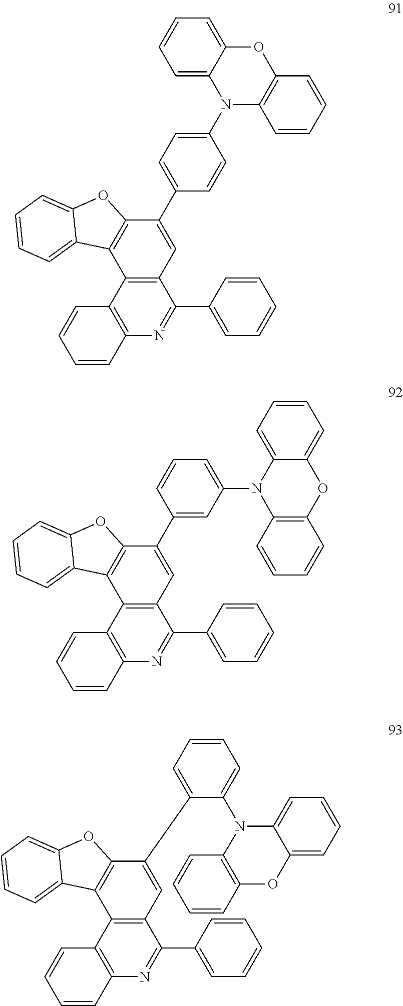

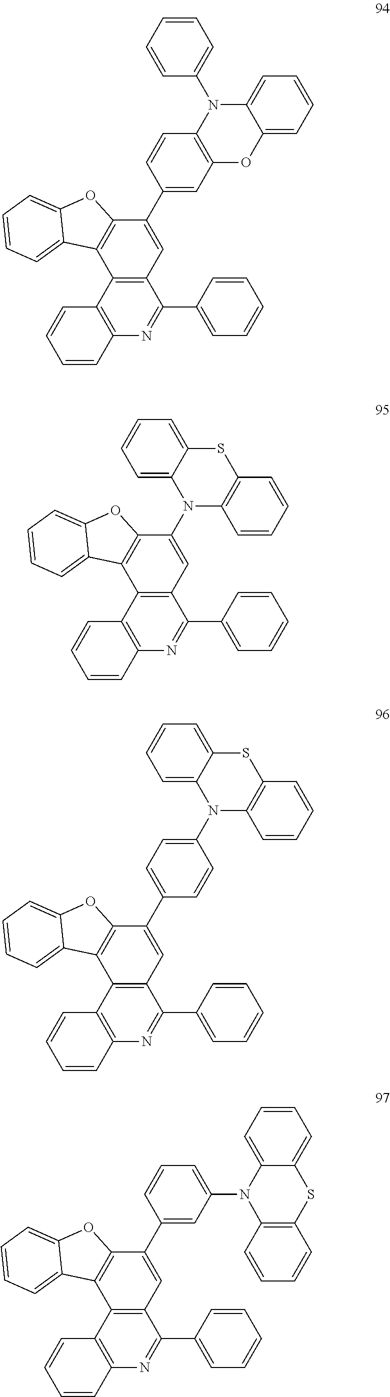

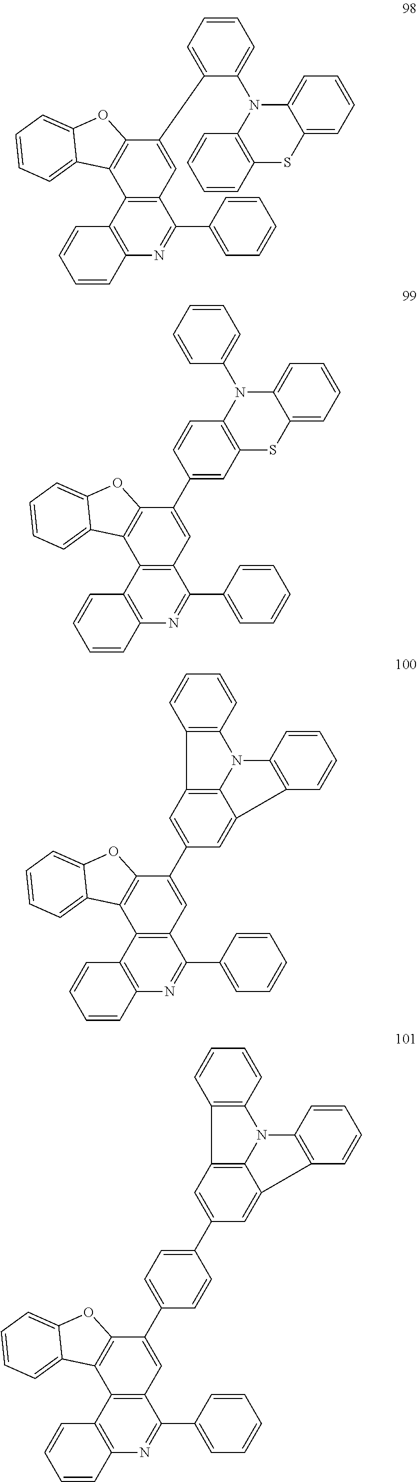 [SMILES: c1ccc(-c2nc3ccccc3c3c2cc(-c2ccccc2N2c4ccccc4Sc4ccccc42)c2oc4ccccc4c23)cc1 ; c1ccc(-c2nc3ccccc3c3c2cc(-c2ccc4c(c2)Sc2ccccc2N4c2ccccc2)c2oc4ccccc4c23)cc1 ; c1ccc(-c2nc3ccccc3c3c2cc(-c2cc4c5ccccc5n5c6ccccc6c(c2)c45)c2oc4ccccc4c23)cc1 ; c1ccc(-c2nc3ccccc3c3c2cc(-c2ccc(-c4cc5c6ccccc6n6c7ccccc7c(c4)c56)cc2)c2oc4ccccc4c23)cc1]

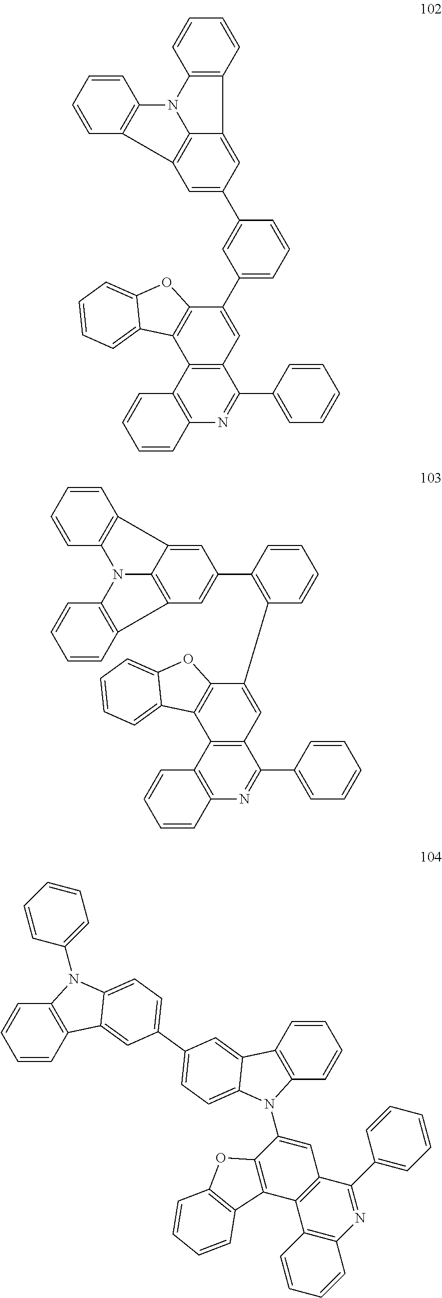 [SMILES: c1ccc(-c2nc3ccccc3c3c2cc(-c2cccc(-c4cc5c6ccccc6n6c7ccccc7c(c4)c56)c2)c2oc4ccccc4c23)cc1 ; c1ccc(-c2nc3ccccc3c3c2cc(-c2ccccc2-c2cc4c5ccccc5n5c6ccccc6c(c2)c45)c2oc4ccccc4c23)cc1 ; c1ccc(-c2nc3ccccc3c3c2cc(-n2c4ccccc4c4cc(-c5ccc6c(c5)c5ccccc5n6-c5ccccc5)ccc42)c2oc4ccccc4c23)cc1]

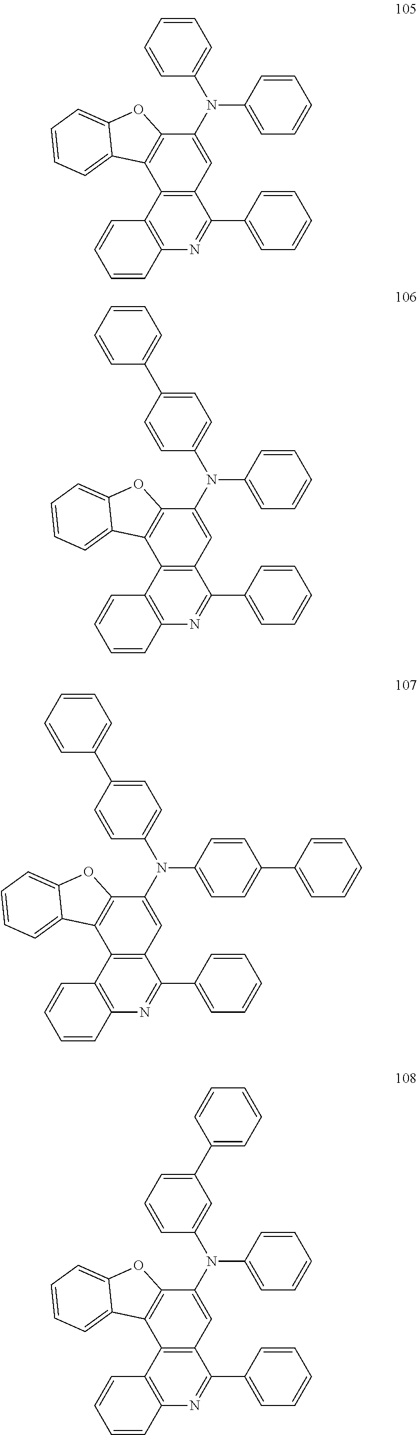

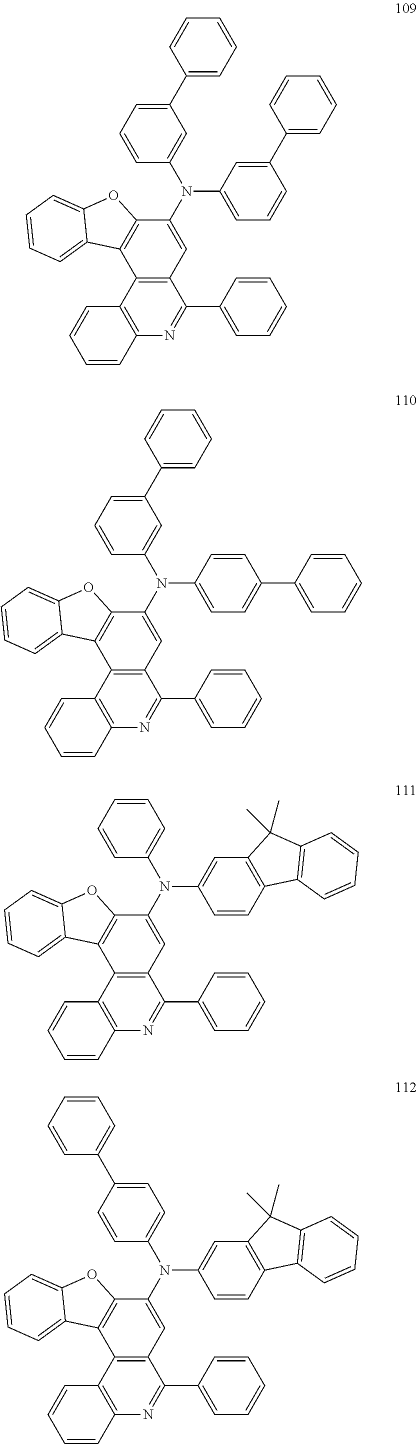

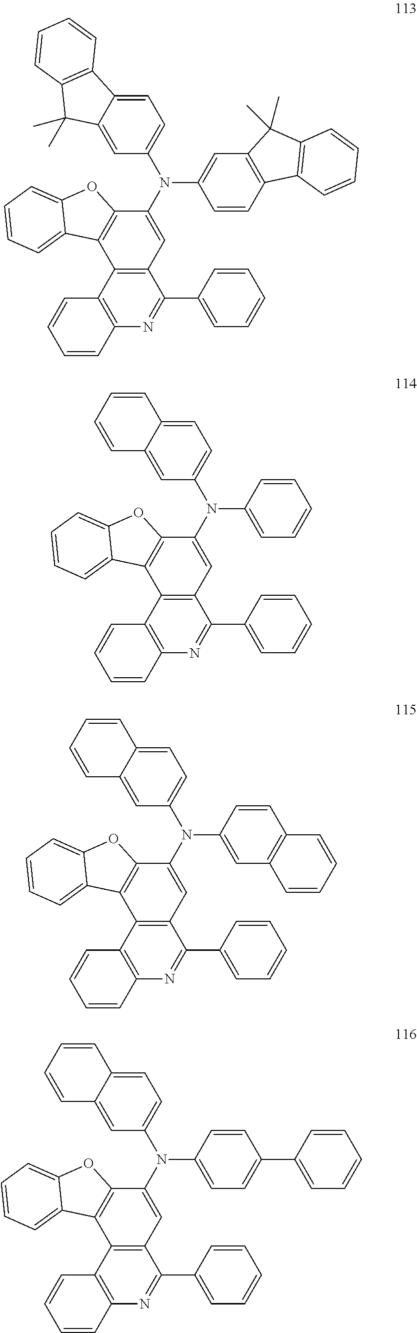

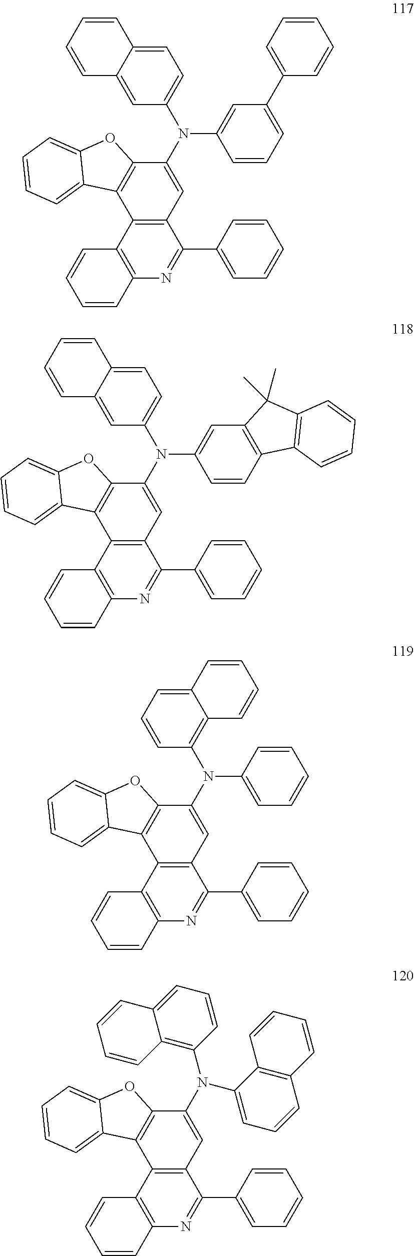

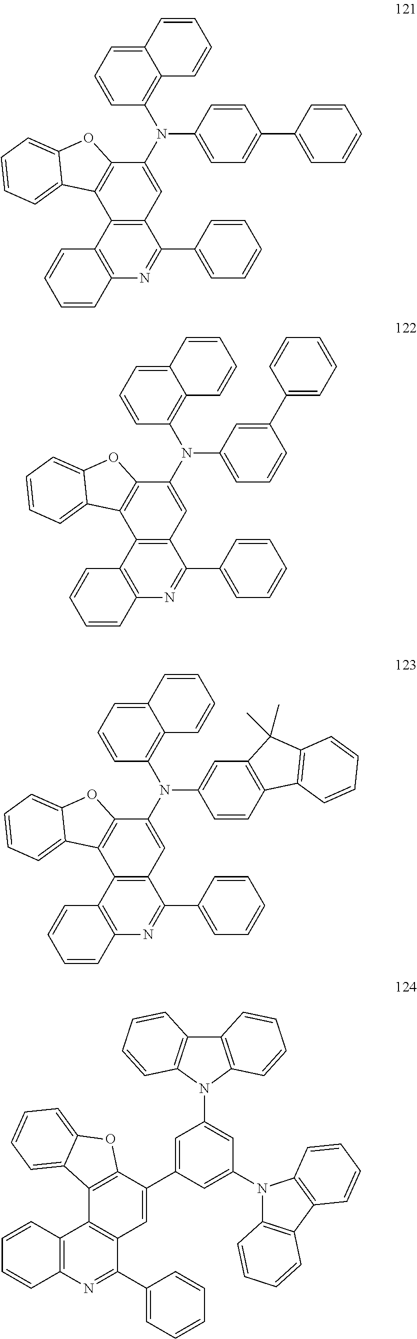 [SMILES: c1ccc(-c2ccc(N(c3cccc4ccccc34)c3cc4c(-c5ccccc5)nc5ccccc5c4c4c3oc3ccccc34)cc2)cc1 ; c1ccc(-c2cccc(N(c3cccc4ccccc34)c3cc4c(-c5ccccc5)nc5ccccc5c4c4c3oc3ccccc34)c2)cc1 ; CC1(C)c2ccccc2-c2ccc(N(c3cccc4ccccc34)c3cc4c(-c5ccccc5)nc5ccccc5c4c4c3oc3ccccc34)cc21 ; c1ccc(-c2nc3ccccc3c3c2cc(-c2cc(-n4c5ccccc5c5ccccc54)cc(-n4c5ccccc5c5ccccc54)c2)c2oc4ccccc4c23)cc1]

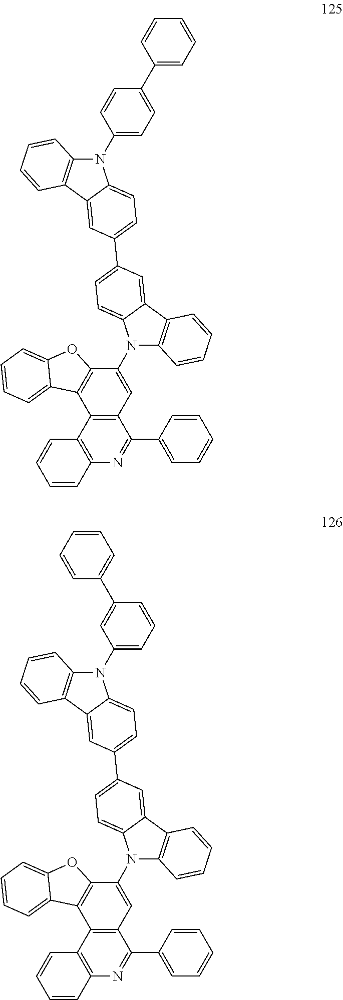 [SMILES: c1ccc(-c2ccc(-n3c4ccccc4c4cc(-c5ccc6c(c5)c5ccccc5n6-c5cc6c(-c7ccccc7)nc7ccccc7c6c6c5oc5ccccc56)ccc43)cc2)cc1 ; c1ccc(-c2cccc(-n3c4ccccc4c4cc(-c5ccc6c(c5)c5ccccc5n6-c5cc6c(-c7ccccc7)nc7ccccc7c6c6c5oc5ccccc56)ccc43)c2)cc1]

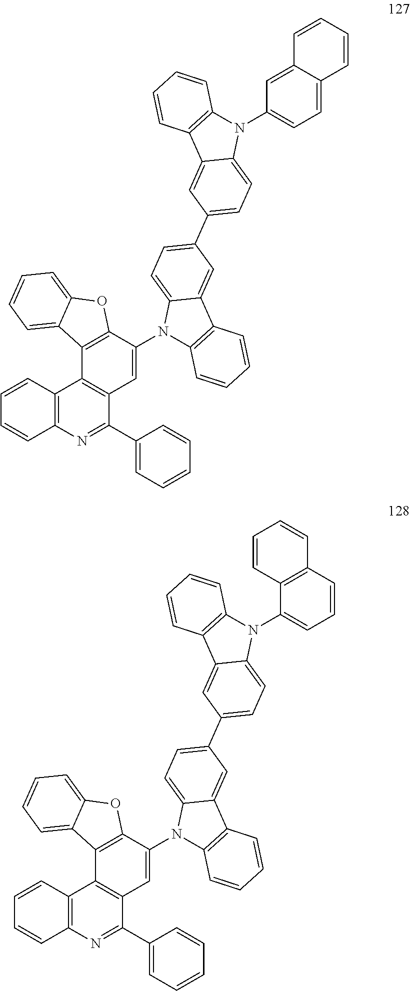 [SMILES: c1ccc(-c2nc3ccccc3c3c2cc(-n2c4ccccc4c4cc(-c5ccc6c(c5)c5ccccc5n6-c5ccc6ccccc6c5)ccc42)c2oc4ccccc4c23)cc1 ; c1ccc(-c2nc3ccccc3c3c2cc(-n2c4ccccc4c4cc(-c5ccc6c(c5)c5ccccc5n6-c5cccc6ccccc56)ccc42)c2oc4ccccc4c23)cc1]

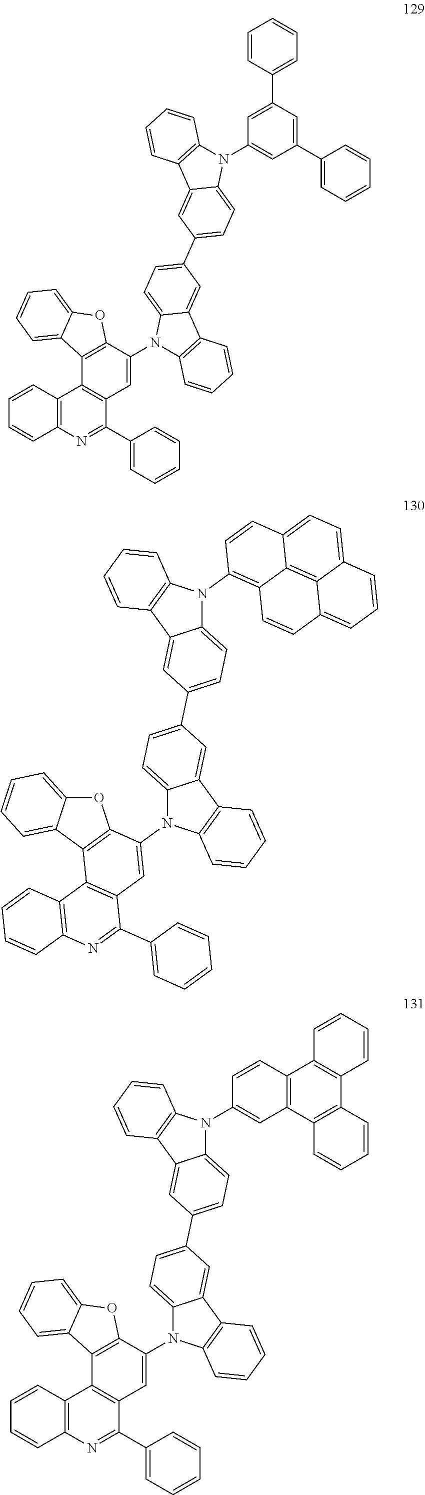 [SMILES: c1ccc(-c2cc(-c3ccccc3)cc(-n3c4ccccc4c4cc(-c5ccc6c(c5)c5ccccc5n6-c5cc6c(-c7ccccc7)nc7ccccc7c6c6c5oc5ccccc56)ccc43)c2)cc1 ; c1ccc(-c2nc3ccccc3c3c2cc(-n2c4ccccc4c4cc(-c5ccc6c(c5)c5ccccc5n6-c5ccc6ccc7cccc8ccc5c6c78)ccc42)c2oc4ccccc4c23)cc1 ; c1ccc(-c2nc3ccccc3c3c2cc(-n2c4ccccc4c4cc(-c5ccc6c(c5)c5ccccc5n6-c5ccc6c7ccccc7c7ccccc7c6c5)ccc42)c2oc4ccccc4c23)cc1]

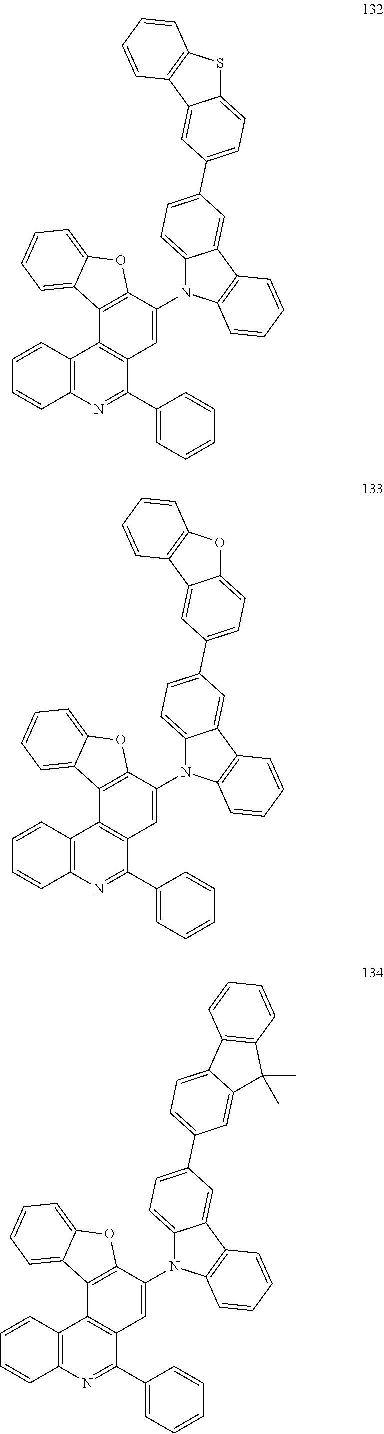

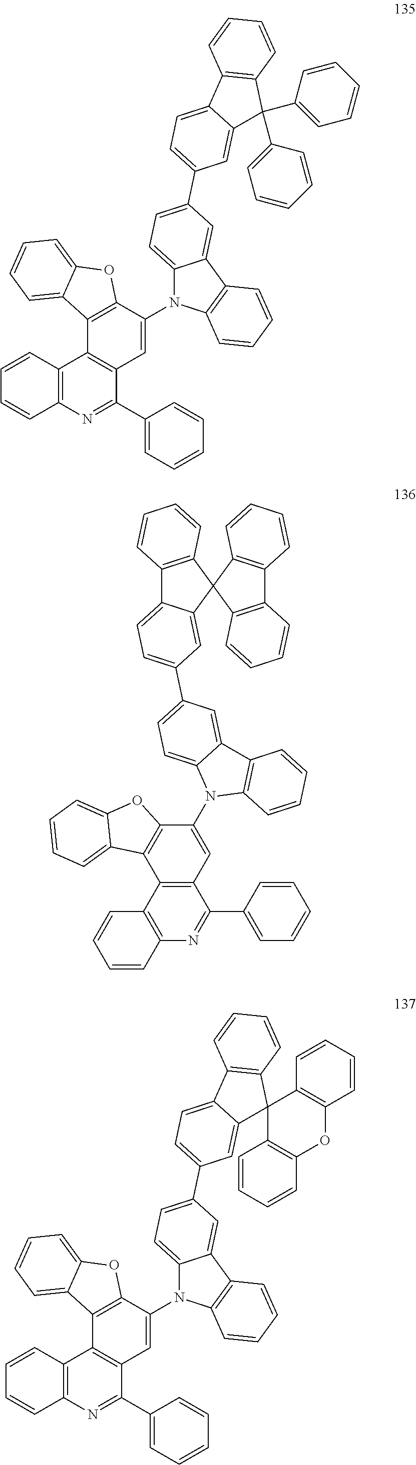

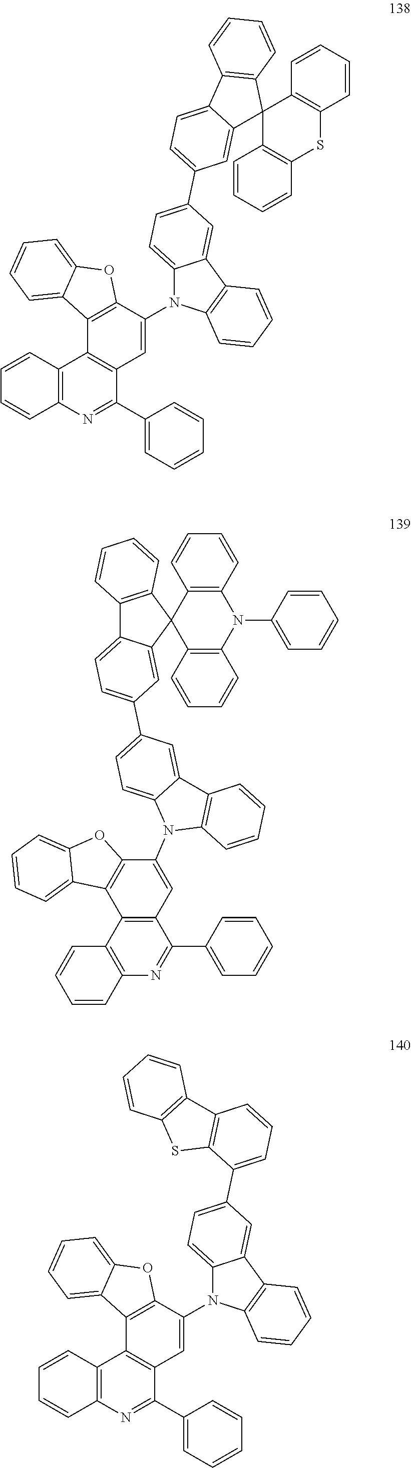 [SMILES: c1ccc(-c2nc3ccccc3c3c2cc(-n2c4ccccc4c4cc(-c5ccc6c(c5)C5(c7ccccc7Sc7ccccc75)c5ccccc5-6)ccc42)c2oc4ccccc4c23)cc1 ; c1ccc(-c2nc3ccccc3c3c2cc(-n2c4ccccc4c4cc(-c5ccc6c(c5)C5(c7ccccc7-6)c6ccccc6N(c6ccccc6)c6ccccc65)ccc42)c2oc4ccccc4c23)cc1 ; c1ccc(-c2nc3ccccc3c3c2cc(-n2c4ccccc4c4cc(-c5cccc6c5sc5ccccc56)ccc42)c2oc4ccccc4c23)cc1]

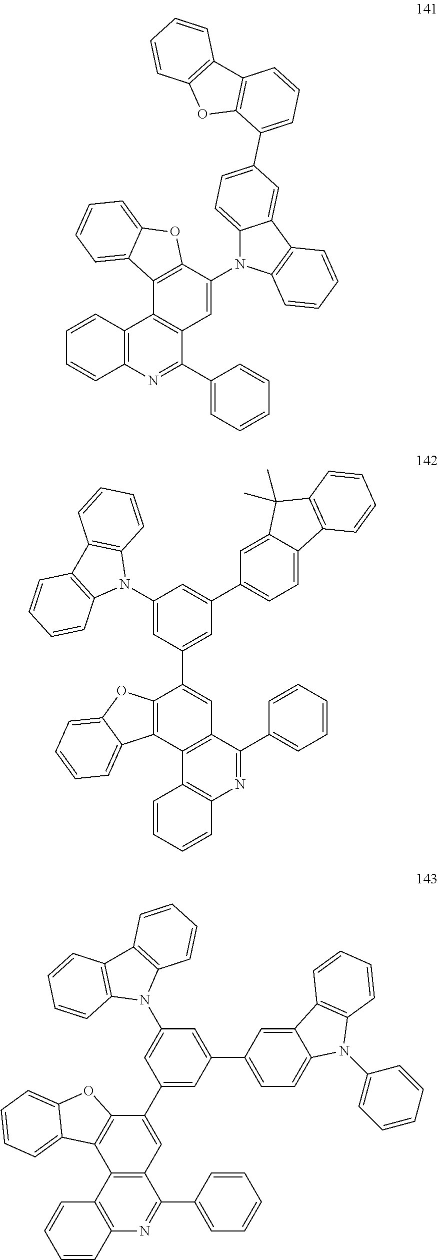 [SMILES: c1ccc(-c2nc3ccccc3c3c2cc(-n2c4ccccc4c4cc(-c5cccc6c5oc5ccccc56)ccc42)c2oc4ccccc4c23)cc1 ; CC1(C)c2ccccc2-c2ccc(-c3cc(-c4cc5c(-c6ccccc6)nc6ccccc6c5c5c4oc4ccccc45)cc(-n4c5ccccc5c5ccccc54)c3)cc21 ; c1ccc(-c2nc3ccccc3c3c2cc(-c2cc(-c4ccc5c(c4)c4ccccc4n5-c4ccccc4)cc(-n4c5ccccc5c5ccccc54)c2)c2oc4ccccc4c23)cc1]

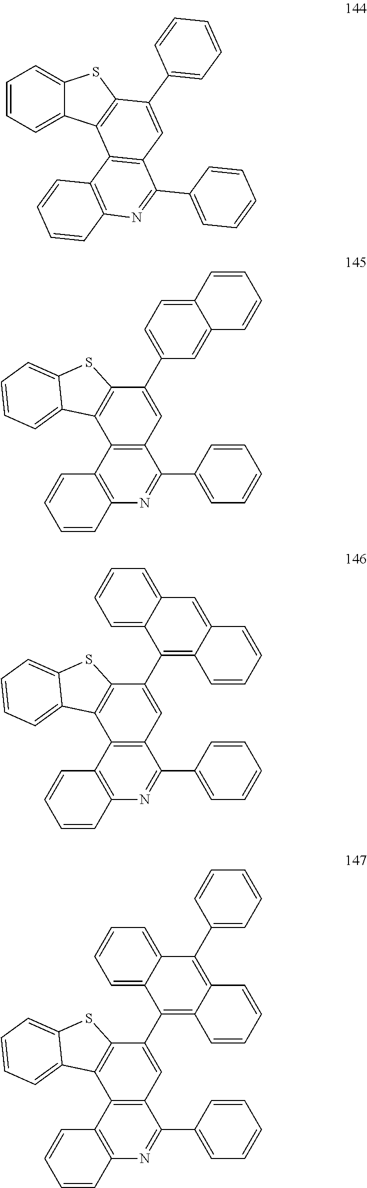

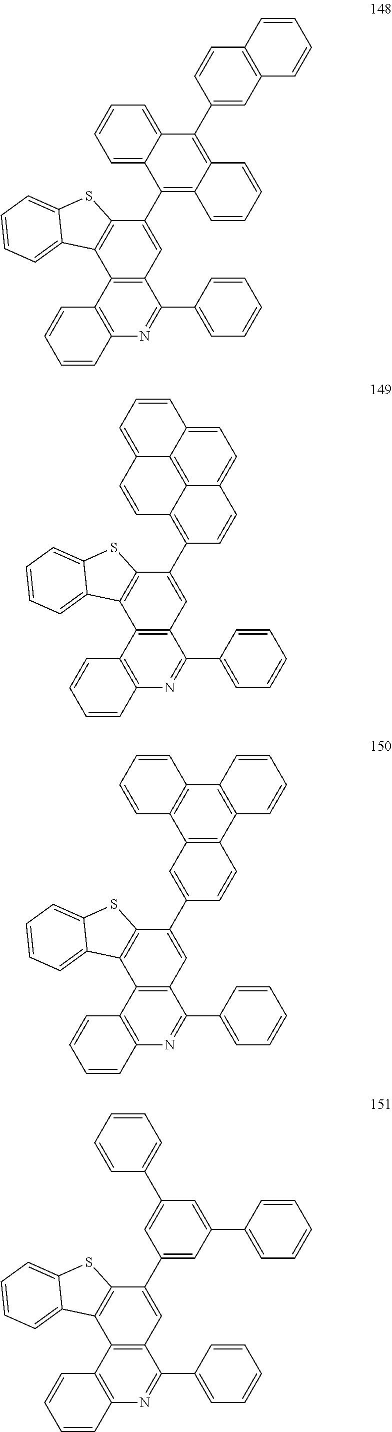

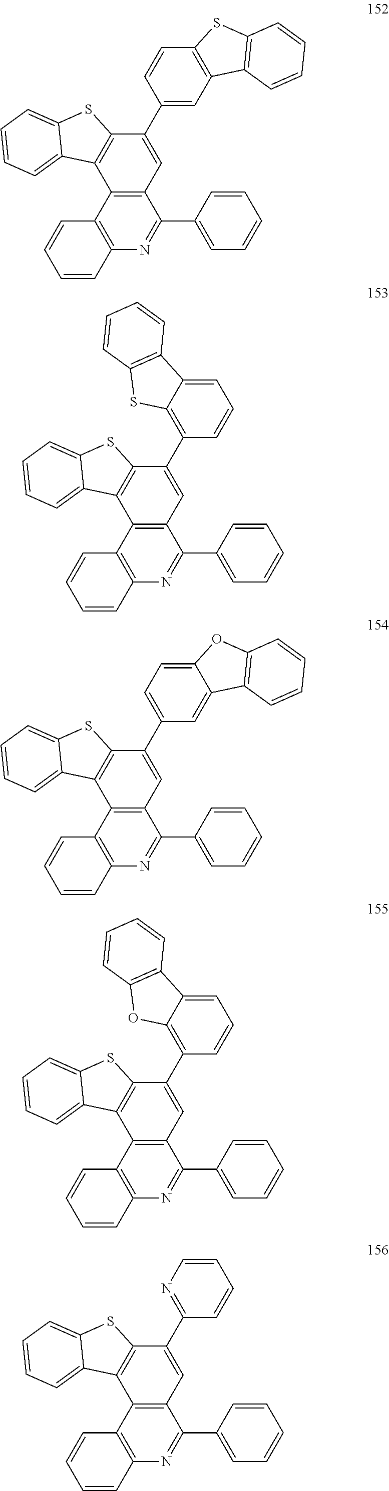

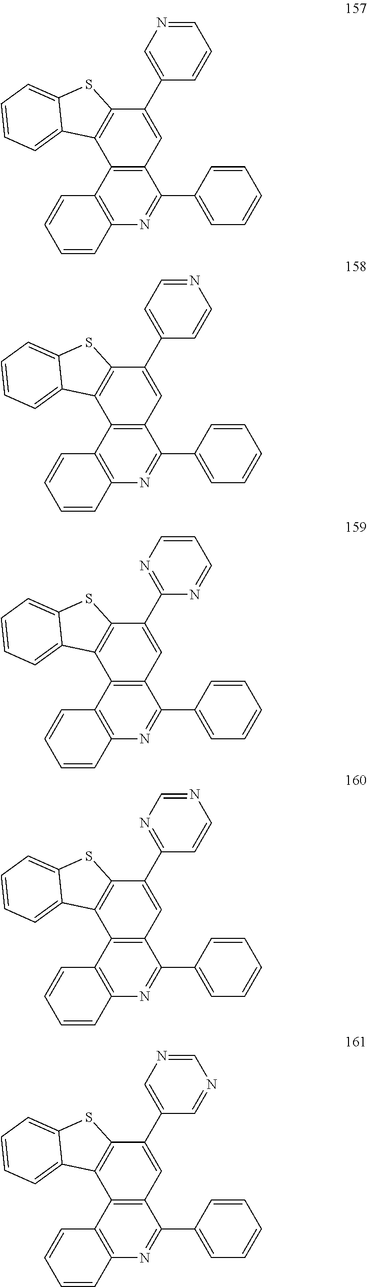 [SMILES: c1ccc(-c2nc3ccccc3c3c2cc(-c2cccnc2)c2sc4ccccc4c23)cc1 ; c1ccc(-c2nc3ccccc3c3c2cc(-c2ccncc2)c2sc4ccccc4c23)cc1 ; c1ccc(-c2nc3ccccc3c3c2cc(-c2ncccn2)c2sc4ccccc4c23)cc1 ; c1ccc(-c2nc3ccccc3c3c2cc(-c2ccncn2)c2sc4ccccc4c23)cc1 ; c1ccc(-c2nc3ccccc3c3c2cc(-c2cncnc2)c2sc4ccccc4c23)cc1]

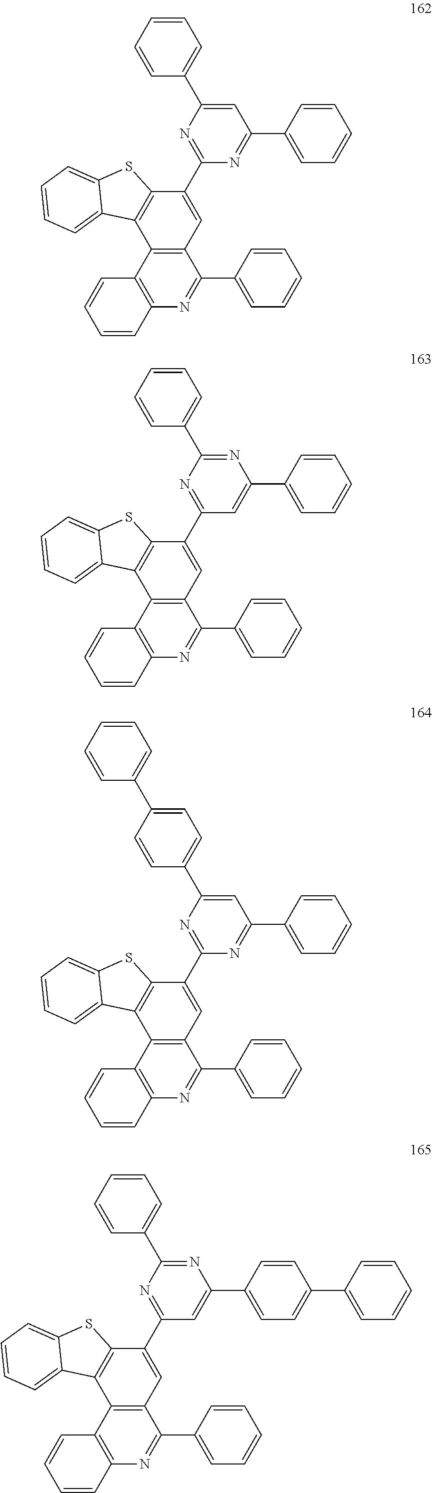

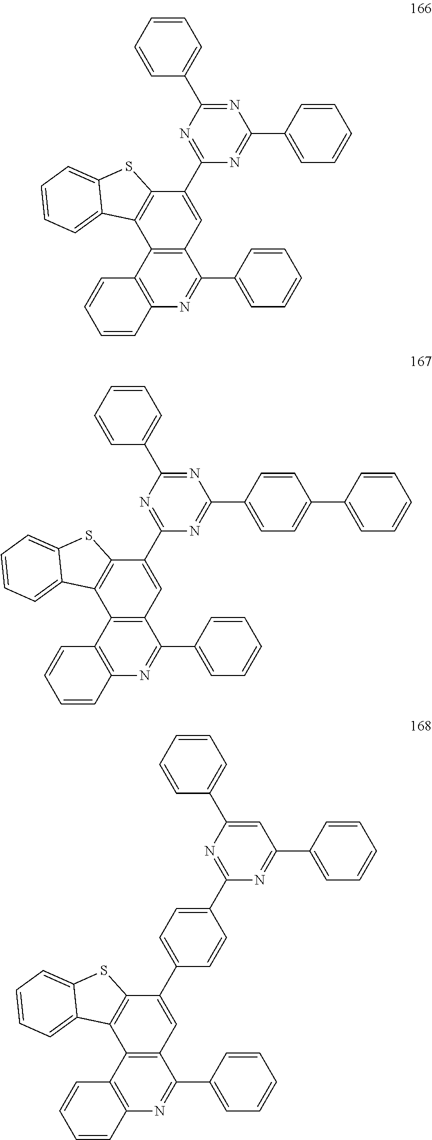 [SMILES: c1ccc(-c2nc(-c3ccccc3)nc(-c3cc4c(-c5ccccc5)nc5ccccc5c4c4c3sc3ccccc34)n2)cc1 ; c1ccc(-c2ccc(-c3nc(-c4ccccc4)nc(-c4cc5c(-c6ccccc6)nc6ccccc6c5c5c4sc4ccccc45)n3)cc2)cc1 ; c1ccc(-c2cc(-c3ccccc3)nc(-c3ccc(-c4cc5c(-c6ccccc6)nc6ccccc6c5c5c4sc4ccccc45)cc3)n2)cc1]

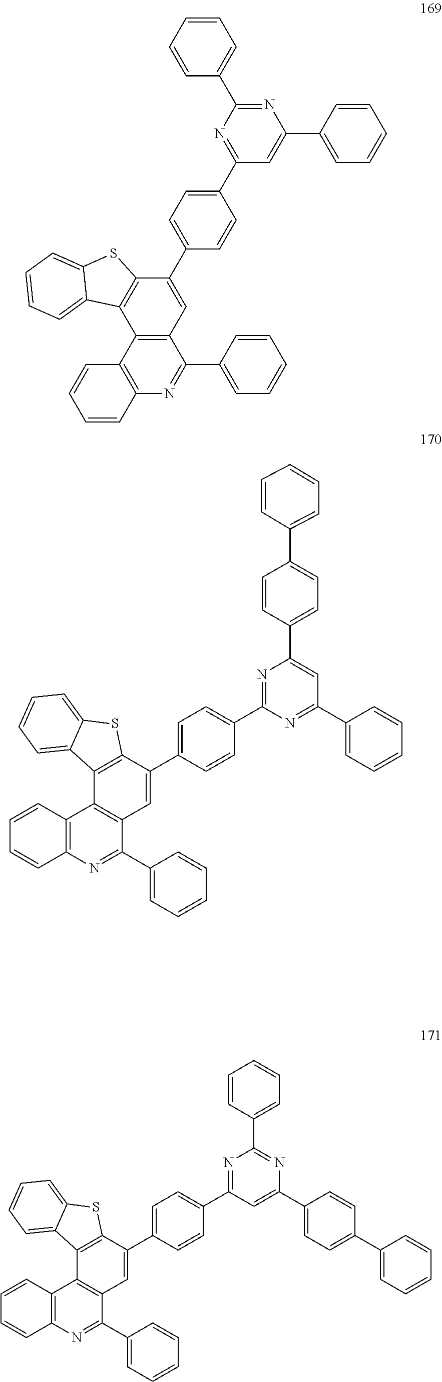 [SMILES: c1ccc(-c2cc(-c3ccc(-c4cc5c(-c6ccccc6)nc6ccccc6c5c5c4sc4ccccc45)cc3)nc(-c3ccccc3)n2)cc1 ; c1ccc(-c2ccc(-c3cc(-c4ccccc4)nc(-c4ccc(-c5cc6c(-c7ccccc7)nc7ccccc7c6c6c5sc5ccccc56)cc4)n3)cc2)cc1 ; c1ccc(-c2ccc(-c3cc(-c4ccc(-c5cc6c(-c7ccccc7)nc7ccccc7c6c6c5sc5ccccc56)cc4)nc(-c4ccccc4)n3)cc2)cc1]

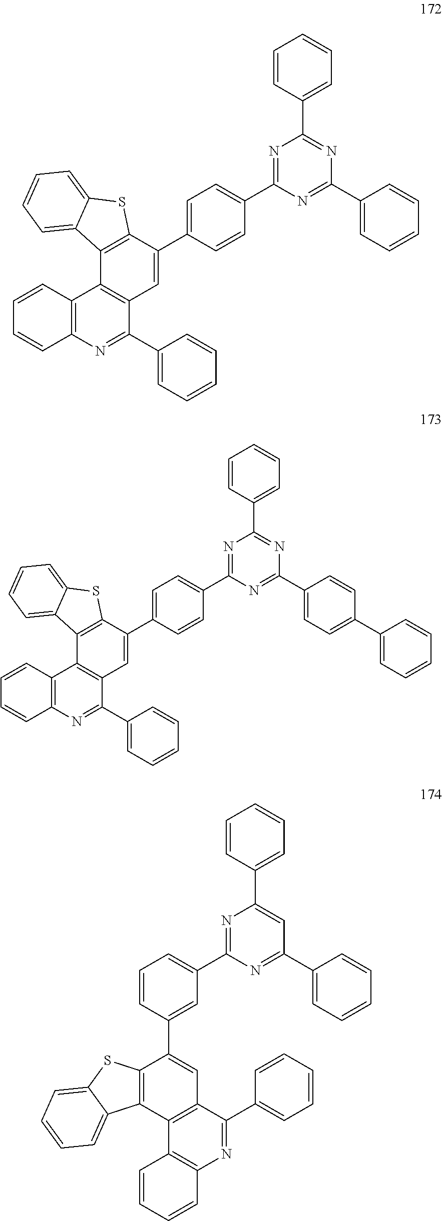 [SMILES: c1ccc(-c2nc(-c3ccccc3)nc(-c3ccc(-c4cc5c(-c6ccccc6)nc6ccccc6c5c5c4sc4ccccc45)cc3)n2)cc1 ; c1ccc(-c2ccc(-c3nc(-c4ccccc4)nc(-c4ccc(-c5cc6c(-c7ccccc7)nc7ccccc7c6c6c5sc5ccccc56)cc4)n3)cc2)cc1 ; c1ccc(-c2cc(-c3ccccc3)nc(-c3cccc(-c4cc5c(-c6ccccc6)nc6ccccc6c5c5c4sc4ccccc45)c3)n2)cc1]

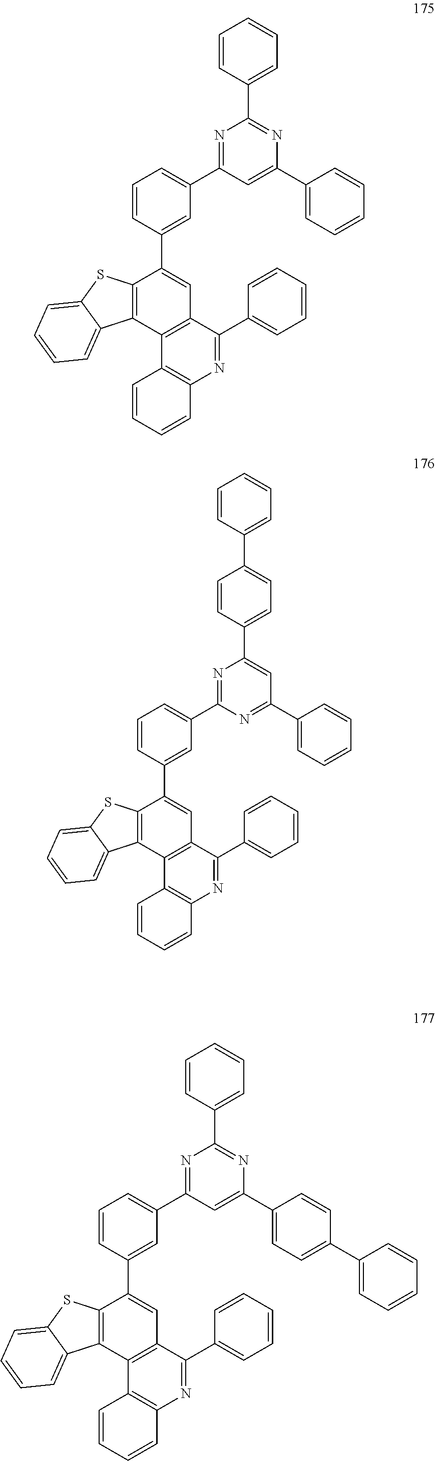 [SMILES: c1ccc(-c2cc(-c3cccc(-c4cc5c(-c6ccccc6)nc6ccccc6c5c5c4sc4ccccc45)c3)nc(-c3ccccc3)n2)cc1 ; c1ccc(-c2ccc(-c3cc(-c4ccccc4)nc(-c4cccc(-c5cc6c(-c7ccccc7)nc7ccccc7c6c6c5sc5ccccc56)c4)n3)cc2)cc1 ; c1ccc(-c2ccc(-c3cc(-c4cccc(-c5cc6c(-c7ccccc7)nc7ccccc7c6c6c5sc5ccccc56)c4)nc(-c4ccccc4)n3)cc2)cc1]

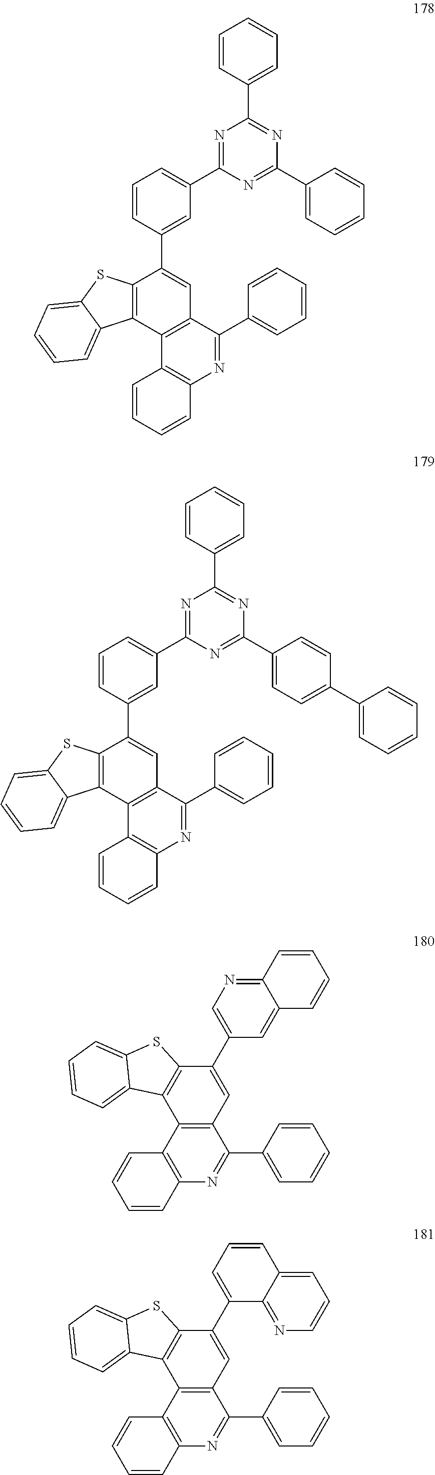 [SMILES: c1ccc(-c2nc(-c3ccccc3)nc(-c3cccc(-c4cc5c(-c6ccccc6)nc6ccccc6c5c5c4sc4ccccc45)c3)n2)cc1 ; c1ccc(-c2ccc(-c3nc(-c4ccccc4)nc(-c4cccc(-c5cc6c(-c7ccccc7)nc7ccccc7c6c6c5sc5ccccc56)c4)n3)cc2)cc1 ; c1ccc(-c2nc3ccccc3c3c2cc(-c2cnc4ccccc4c2)c2sc4ccccc4c23)cc1 ; c1ccc(-c2nc3ccccc3c3c2cc(-c2cccc4cccnc24)c2sc4ccccc4c23)cc1]

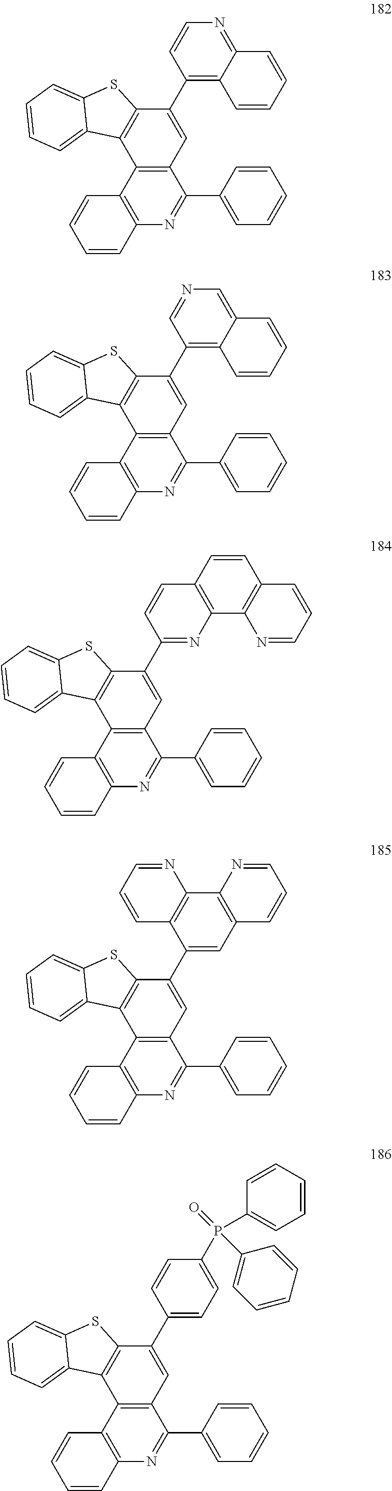 [SMILES: c1ccc(-c2nc3ccccc3c3c2cc(-c2ccnc4ccccc24)c2sc4ccccc4c23)cc1 ; c1ccc(-c2nc3ccccc3c3c2cc(-c2cncc4ccccc24)c2sc4ccccc4c23)cc1 ; c1ccc(-c2nc3ccccc3c3c2cc(-c2ccc4ccc5cccnc5c4n2)c2sc4ccccc4c23)cc1 ; c1ccc(-c2nc3ccccc3c3c2cc(-c2cc4cccnc4c4ncccc24)c2sc4ccccc4c23)cc1 ; O=P(c1ccccc1)(c1ccccc1)c1ccc(-c2cc3c(-c4ccccc4)nc4ccccc4c3c3c2sc2ccccc23)cc1]

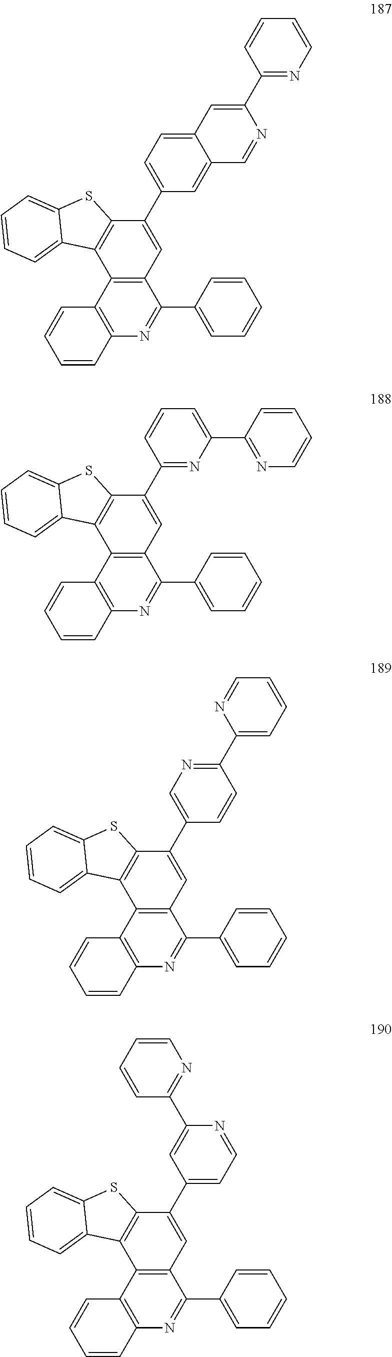

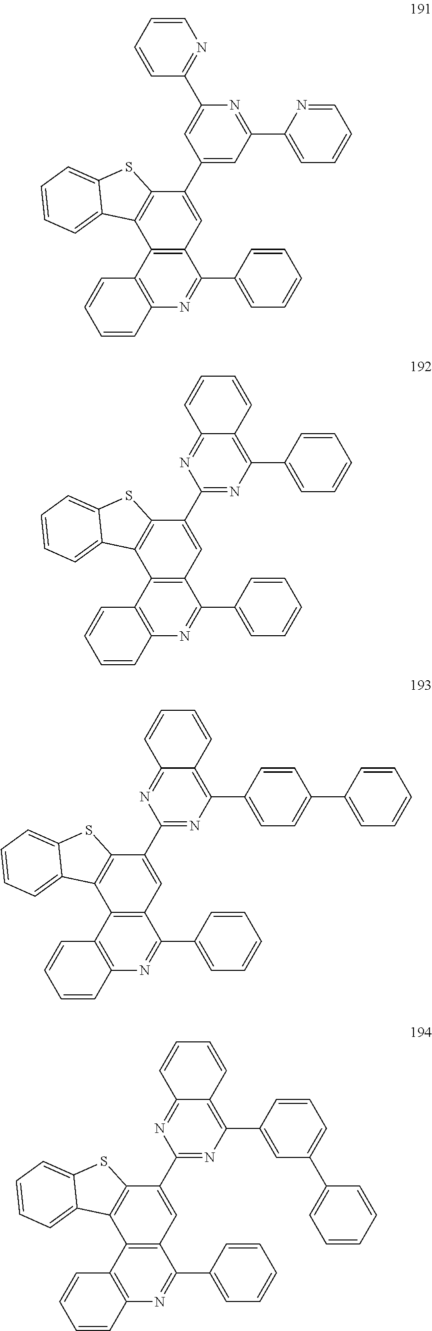 [SMILES: c1ccc(-c2nc3ccccc3c3c2cc(-c2cc(-c4ccccn4)nc(-c4ccccn4)c2)c2sc4ccccc4c23)cc1 ; c1ccc(-c2nc(-c3cc4c(-c5ccccc5)nc5ccccc5c4c4c3sc3ccccc34)nc3ccccc23)cc1 ; c1ccc(-c2ccc(-c3nc(-c4cc5c(-c6ccccc6)nc6ccccc6c5c5c4sc4ccccc45)nc4ccccc34)cc2)cc1 ; c1ccc(-c2cccc(-c3nc(-c4cc5c(-c6ccccc6)nc6ccccc6c5c5c4sc4ccccc45)nc4ccccc34)c2)cc1]

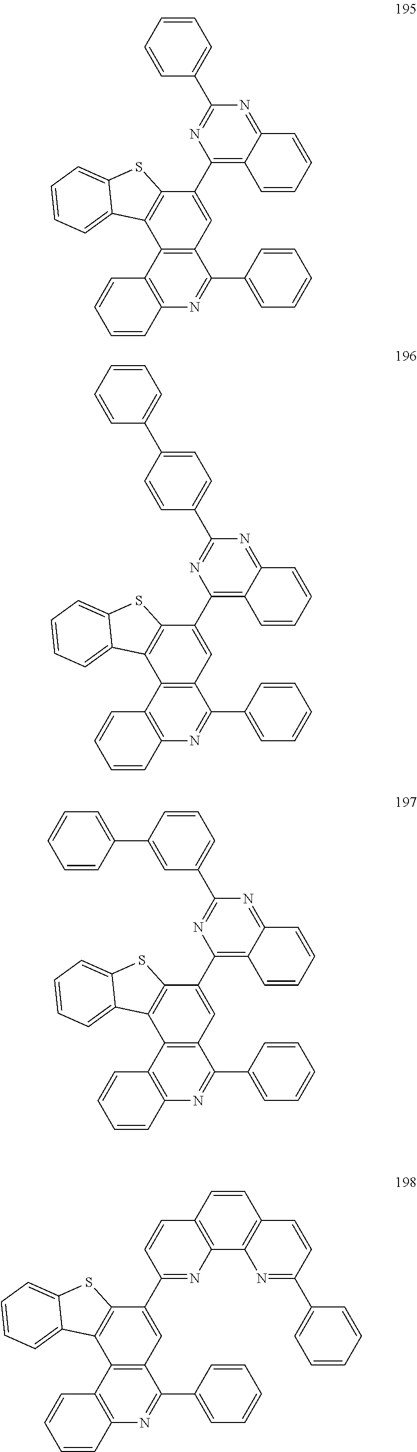

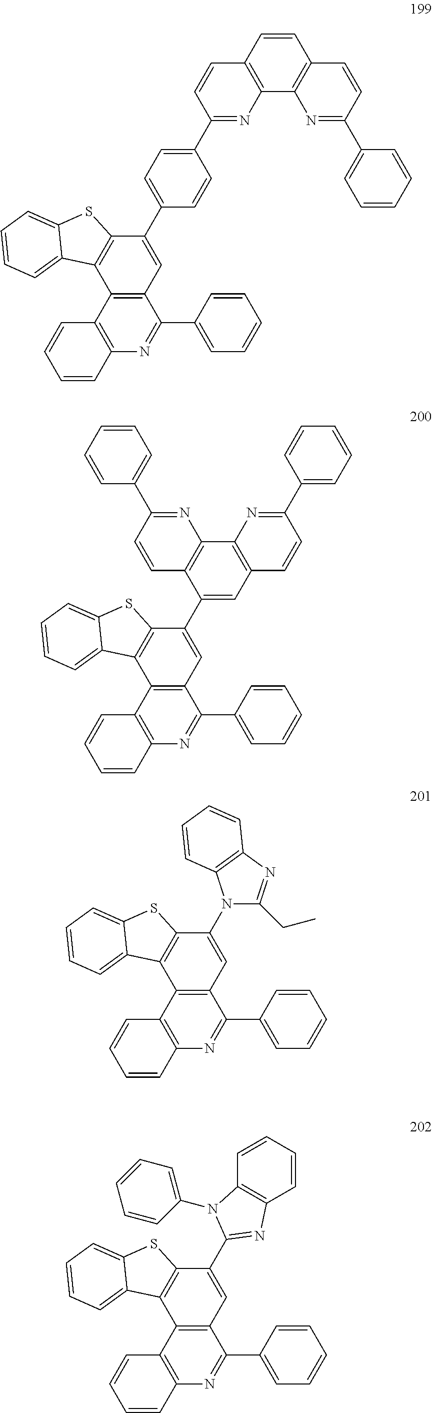

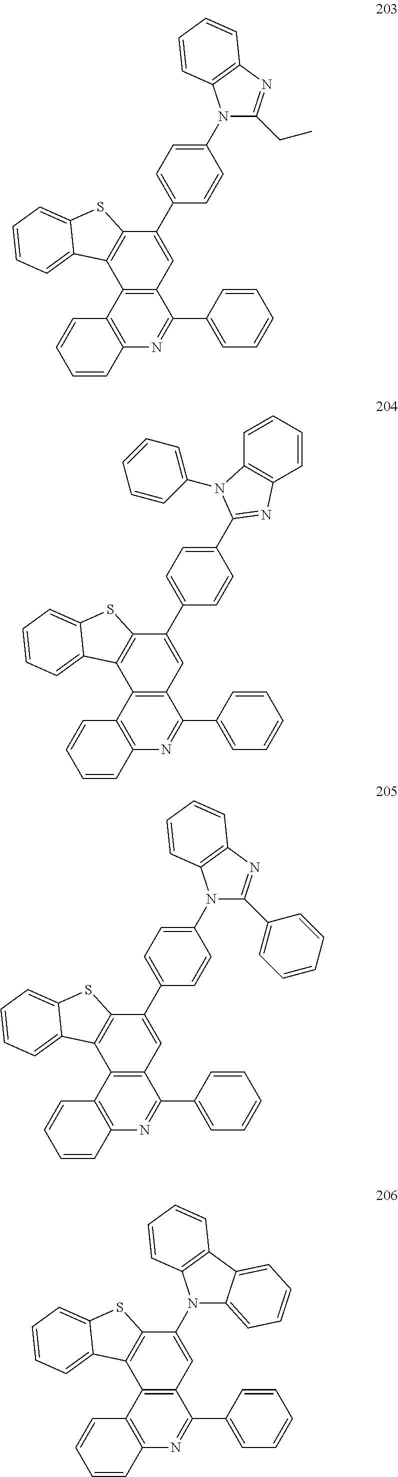 [SMILES: CCc1nc2ccccc2n1-c1ccc(-c2cc3c(-c4ccccc4)nc4ccccc4c3c3c2sc2ccccc23)cc1 ; c1ccc(-c2nc3ccccc3c3c2cc(-c2ccc(-c4nc5ccccc5n4-c4ccccc4)cc2)c2sc4ccccc4c23)cc1 ; c1ccc(-c2nc3ccccc3c3c2cc(-c2ccc(-n4c(-c5ccccc5)nc5ccccc54)cc2)c2sc4ccccc4c23)cc1 ; c1ccc(-c2nc3ccccc3c3c2cc(-n2c4ccccc4c4ccccc42)c2sc4ccccc4c23)cc1]

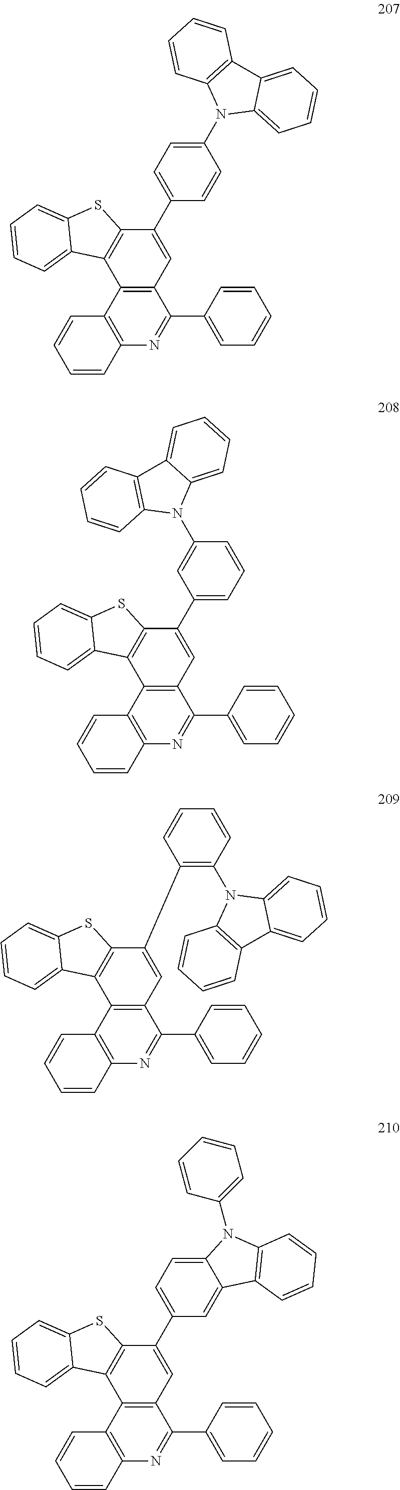 [SMILES: c1ccc(-c2nc3ccccc3c3c2cc(-c2ccc(-n4c5ccccc5c5ccccc54)cc2)c2sc4ccccc4c23)cc1 ; c1ccc(-c2nc3ccccc3c3c2cc(-c2cccc(-n4c5ccccc5c5ccccc54)c2)c2sc4ccccc4c23)cc1 ; c1ccc(-c2nc3ccccc3c3c2cc(-c2ccccc2-n2c4ccccc4c4ccccc42)c2sc4ccccc4c23)cc1 ; c1ccc(-c2nc3ccccc3c3c2cc(-c2ccc4c(c2)c2ccccc2n4-c2ccccc2)c2sc4ccccc4c23)cc1]

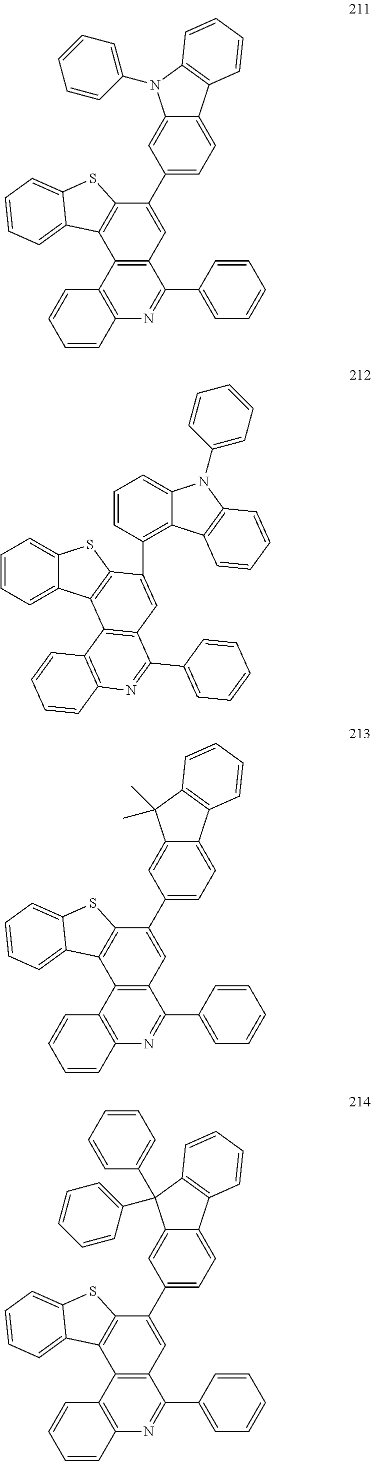

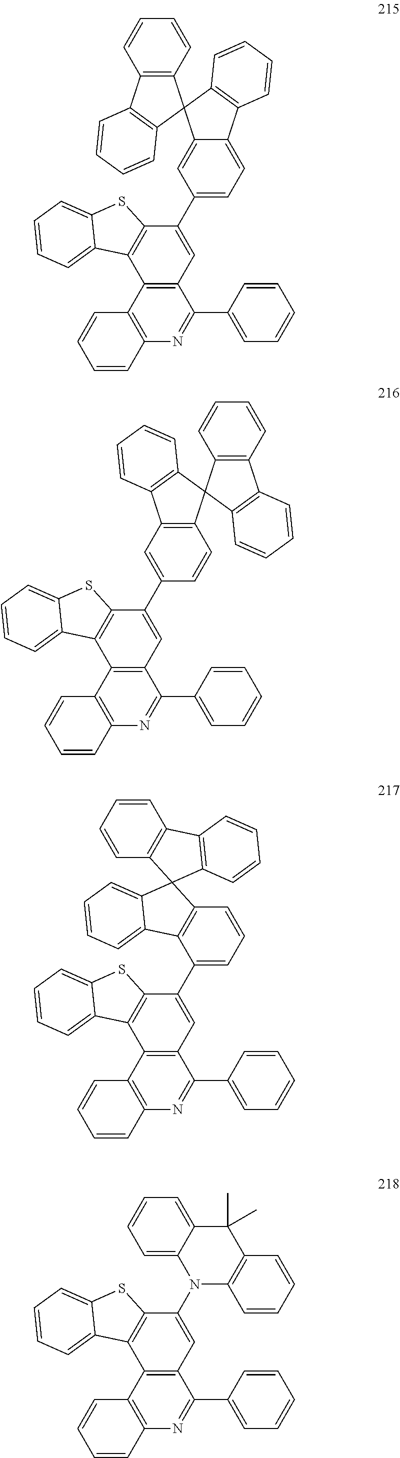

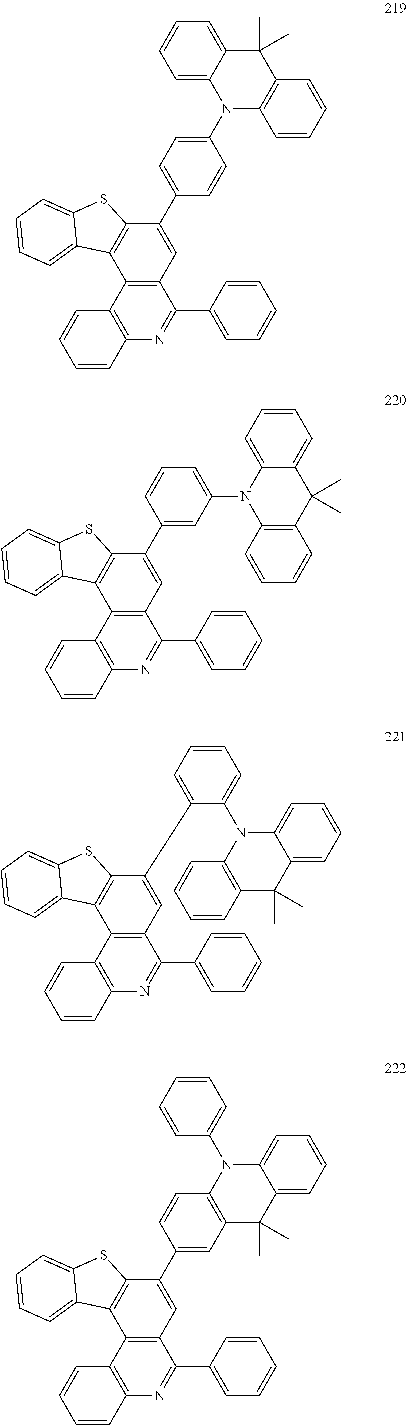 [SMILES: CC1(C)c2ccccc2N(c2ccc(-c3cc4c(-c5ccccc5)nc5ccccc5c4c4c3sc3ccccc34)cc2)c2ccccc21 ; CC1(C)c2ccccc2N(c2cccc(-c3cc4c(-c5ccccc5)nc5ccccc5c4c4c3sc3ccccc34)c2)c2ccccc21 ; CC1(C)c2ccccc2N(c2ccccc2-c2cc3c(-c4ccccc4)nc4ccccc4c3c3c2sc2ccccc23)c2ccccc21 ; CC1(C)c2ccccc2N(c2ccccc2)c2ccc(-c3cc4c(-c5ccccc5)nc5ccccc5c4c4c3sc3ccccc34)cc21]

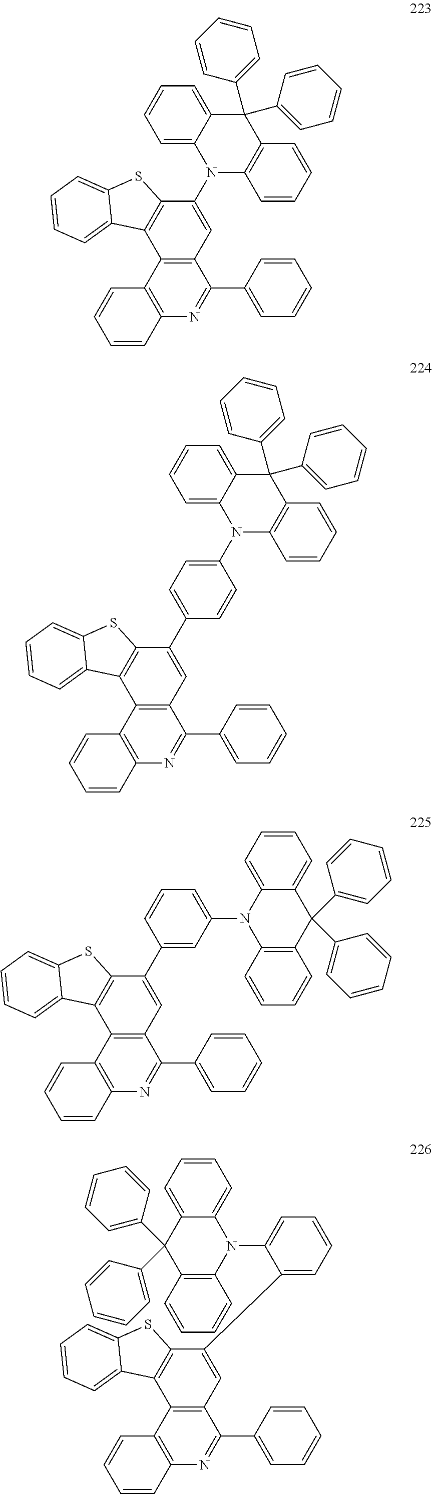 [SMILES: c1ccc(-c2nc3ccccc3c3c2cc(N2c4ccccc4C(c4ccccc4)(c4ccccc4)c4ccccc42)c2sc4ccccc4c23)cc1 ; c1ccc(-c2nc3ccccc3c3c2cc(-c2ccc(N4c5ccccc5C(c5ccccc5)(c5ccccc5)c5ccccc54)cc2)c2sc4ccccc4c23)cc1 ; c1ccc(-c2nc3ccccc3c3c2cc(-c2cccc(N4c5ccccc5C(c5ccccc5)(c5ccccc5)c5ccccc54)c2)c2sc4ccccc4c23)cc1 ; c1ccc(-c2nc3ccccc3c3c2cc(-c2ccccc2N2c4ccccc4C(c4ccccc4)(c4ccccc4)c4ccccc42)c2sc4ccccc4c23)cc1]

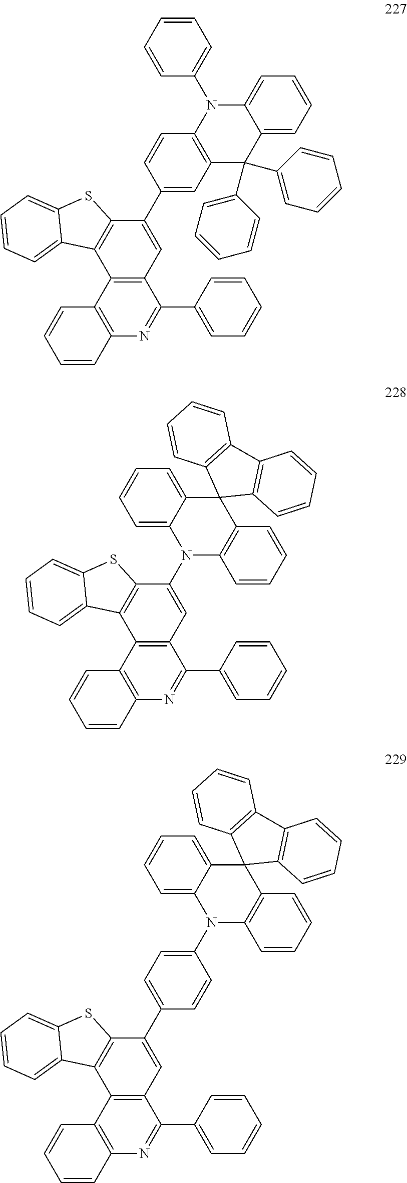 [SMILES: c1ccc(-c2nc3ccccc3c3c2cc(-c2ccc4c(c2)C(c2ccccc2)(c2ccccc2)c2ccccc2N4c2ccccc2)c2sc4ccccc4c23)cc1 ; c1ccc(-c2nc3ccccc3c3c2cc(N2c4ccccc4C4(c5ccccc5-c5ccccc54)c4ccccc42)c2sc4ccccc4c23)cc1 ; c1ccc(-c2nc3ccccc3c3c2cc(-c2ccc(N4c5ccccc5C5(c6ccccc6-c6ccccc65)c5ccccc54)cc2)c2sc4ccccc4c23)cc1]

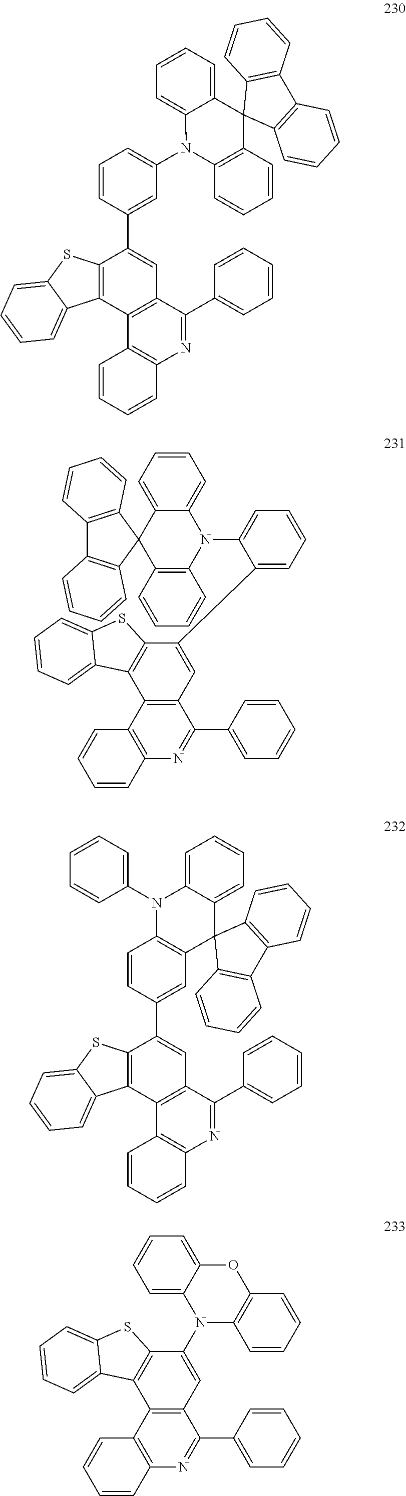

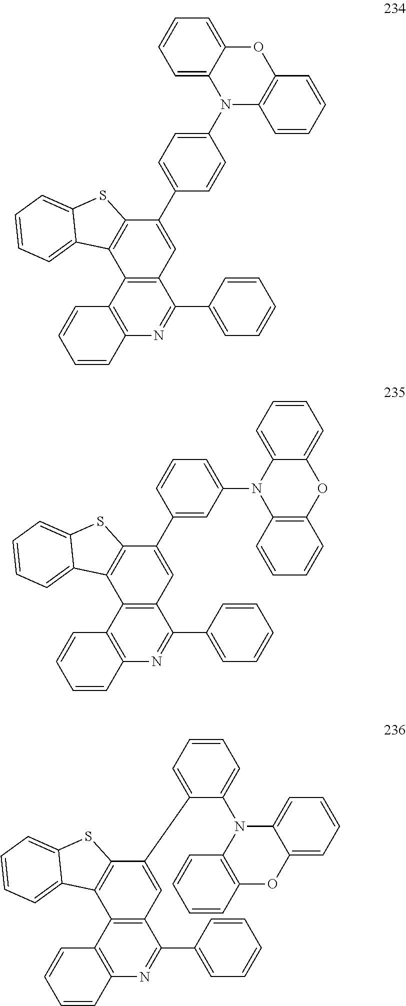

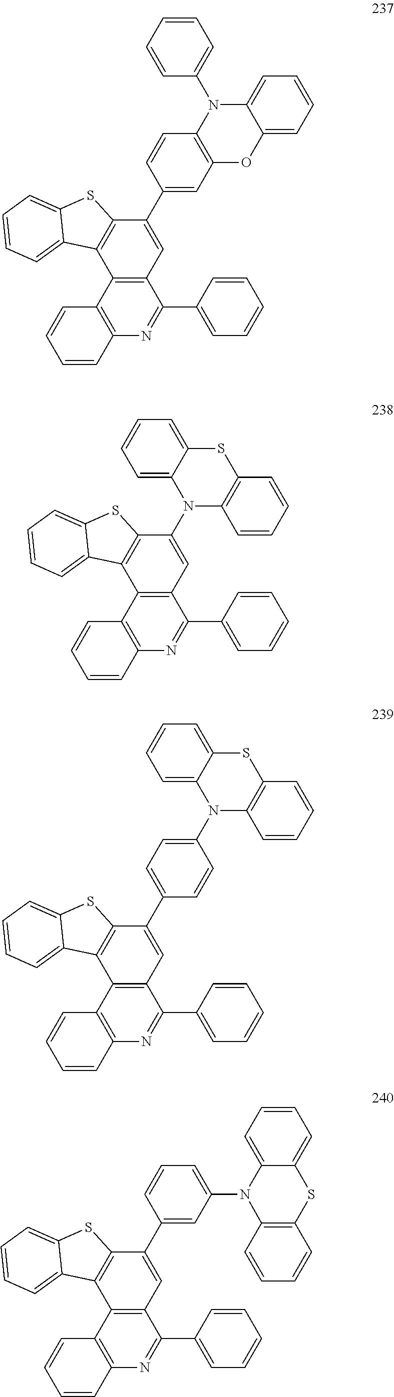

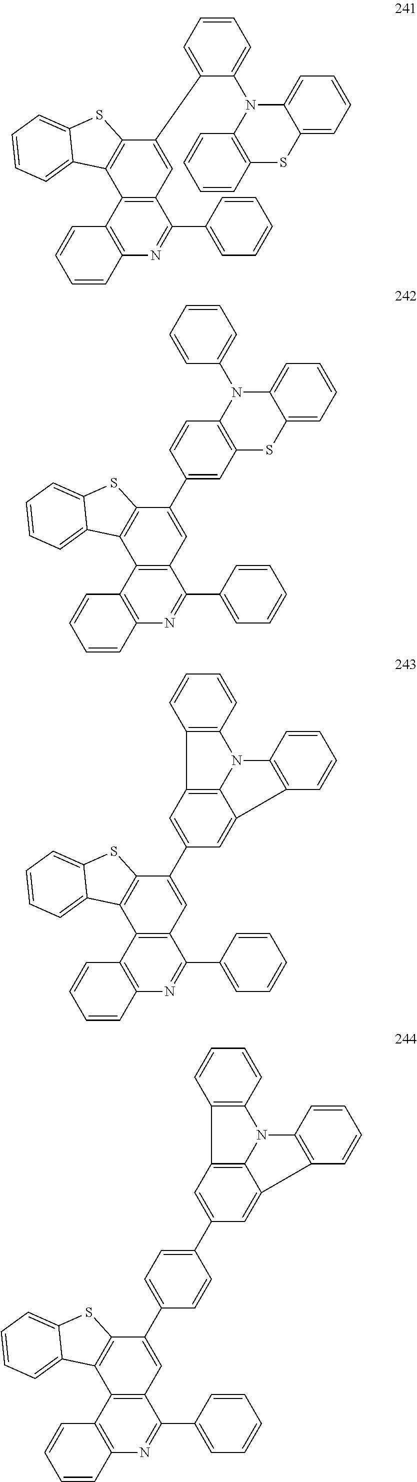 [SMILES: c1ccc(-c2nc3ccccc3c3c2cc(-c2ccccc2N2c4ccccc4Sc4ccccc42)c2sc4ccccc4c23)cc1 ; c1ccc(-c2nc3ccccc3c3c2cc(-c2ccc4c(c2)Sc2ccccc2N4c2ccccc2)c2sc4ccccc4c23)cc1 ; c1ccc(-c2nc3ccccc3c3c2cc(-c2cc4c5ccccc5n5c6ccccc6c(c2)c45)c2sc4ccccc4c23)cc1 ; c1ccc(-c2nc3ccccc3c3c2cc(-c2ccc(-c4cc5c6ccccc6n6c7ccccc7c(c4)c56)cc2)c2sc4ccccc4c23)cc1]

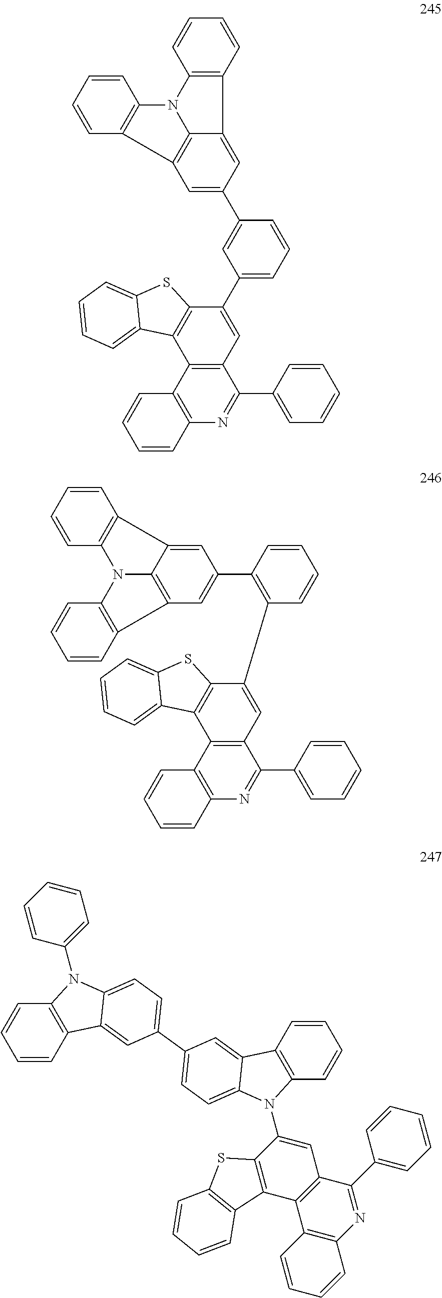 [SMILES: c1ccc(-c2nc3ccccc3c3c2cc(-c2cccc(-c4cc5c6ccccc6n6c7ccccc7c(c4)c56)c2)c2sc4ccccc4c23)cc1 ; c1ccc(-c2nc3ccccc3c3c2cc(-c2ccccc2-c2cc4c5ccccc5n5c6ccccc6c(c2)c45)c2sc4ccccc4c23)cc1 ; c1ccc(-c2nc3ccccc3c3c2cc(-n2c4ccccc4c4cc(-c5ccc6c(c5)c5ccccc5n6-c5ccccc5)ccc42)c2sc4ccccc4c23)cc1]

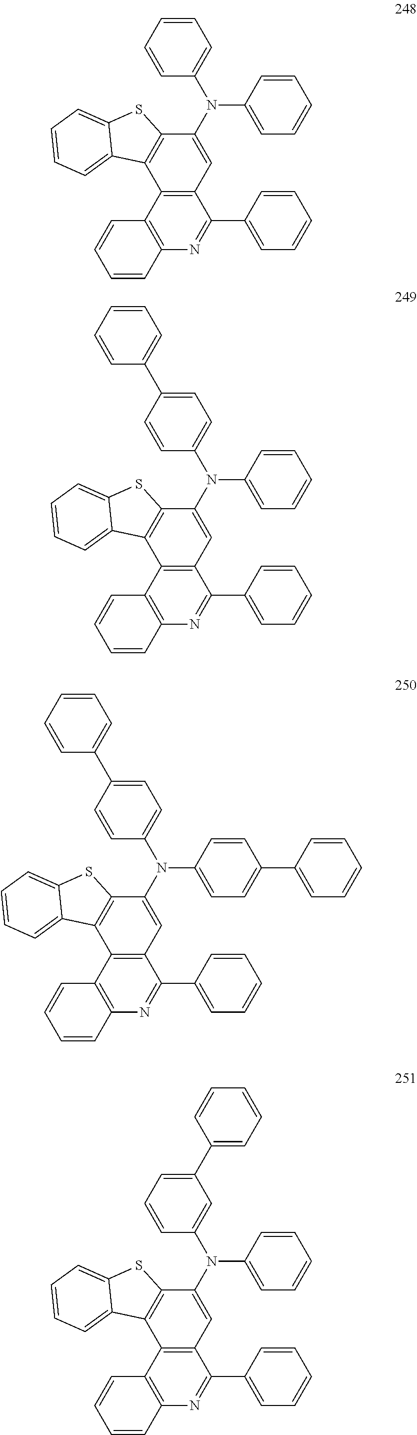

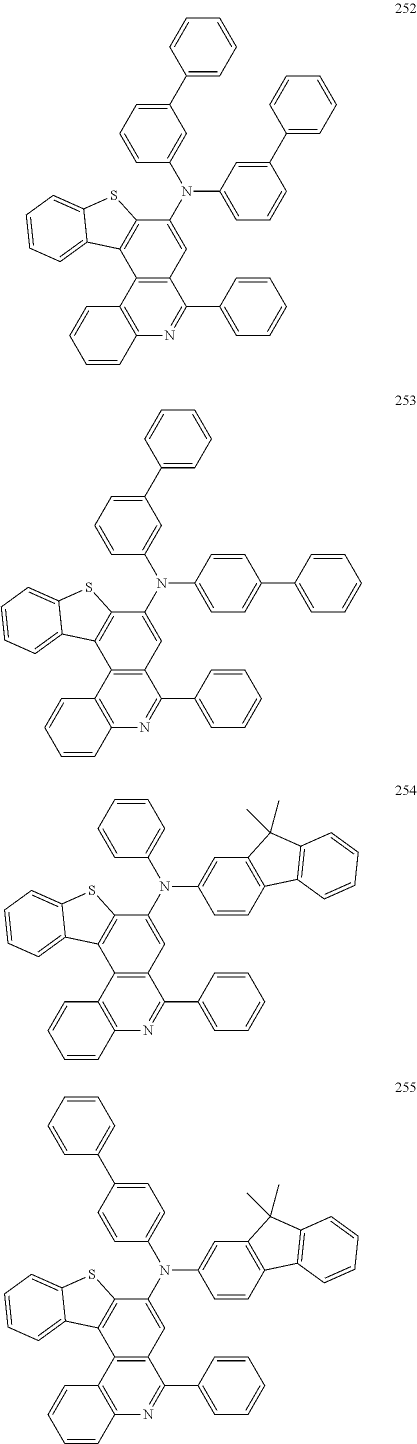

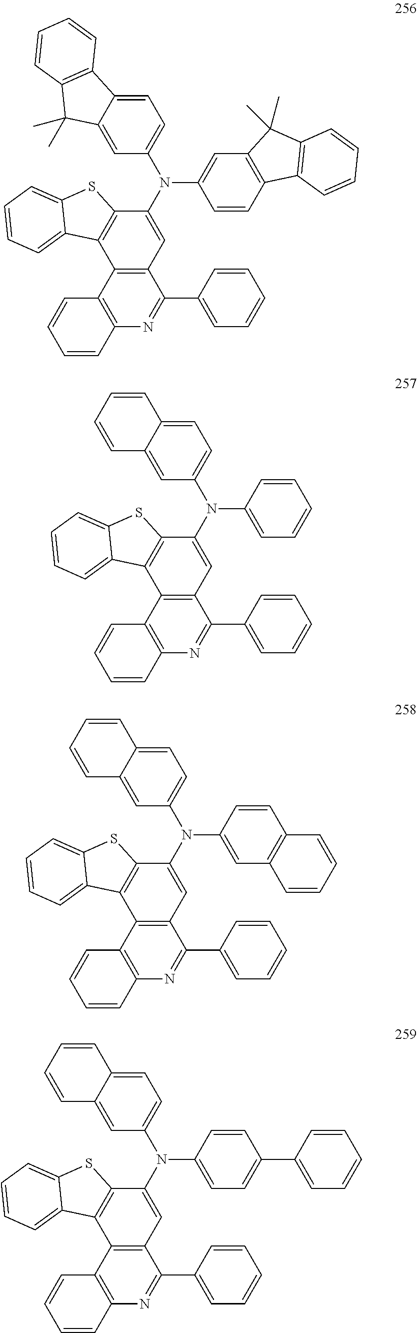

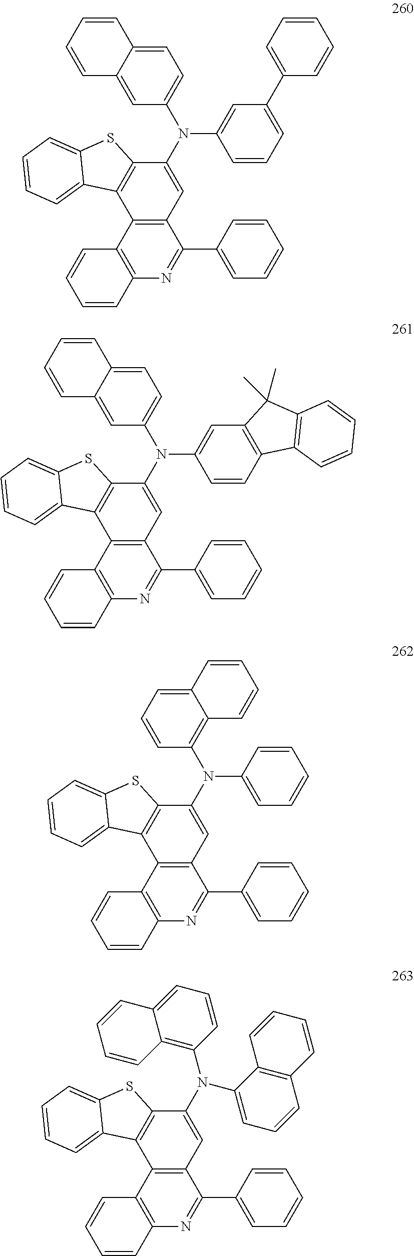

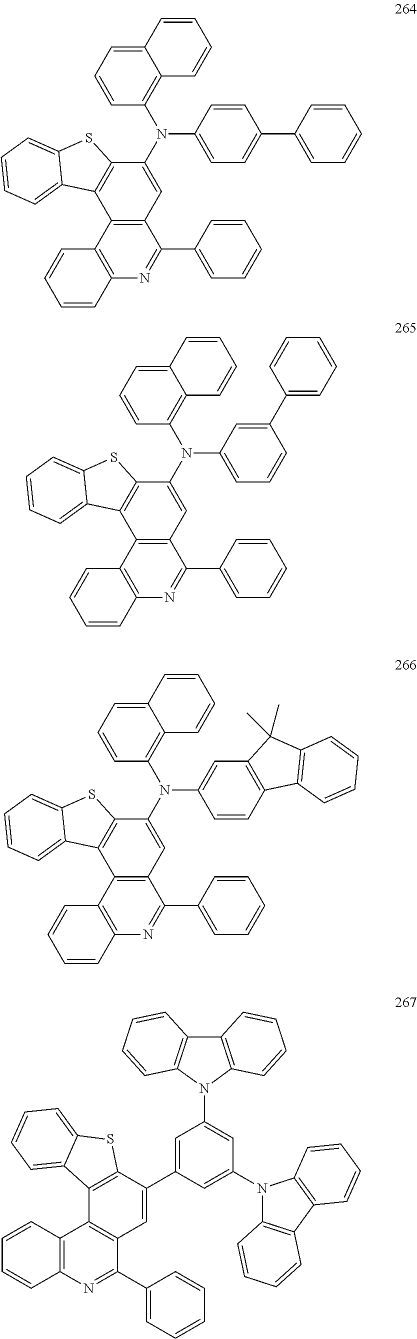 [SMILES: c1ccc(-c2ccc(N(c3cccc4ccccc34)c3cc4c(-c5ccccc5)nc5ccccc5c4c4c3sc3ccccc34)cc2)cc1 ; c1ccc(-c2cccc(N(c3cccc4ccccc34)c3cc4c(-c5ccccc5)nc5ccccc5c4c4c3sc3ccccc34)c2)cc1 ; CC1(C)c2ccccc2-c2ccc(N(c3cccc4ccccc34)c3cc4c(-c5ccccc5)nc5ccccc5c4c4c3sc3ccccc34)cc21 ; c1ccc(-c2nc3ccccc3c3c2cc(-c2cc(-n4c5ccccc5c5ccccc54)cc(-n4c5ccccc5c5ccccc54)c2)c2sc4ccccc4c23)cc1]

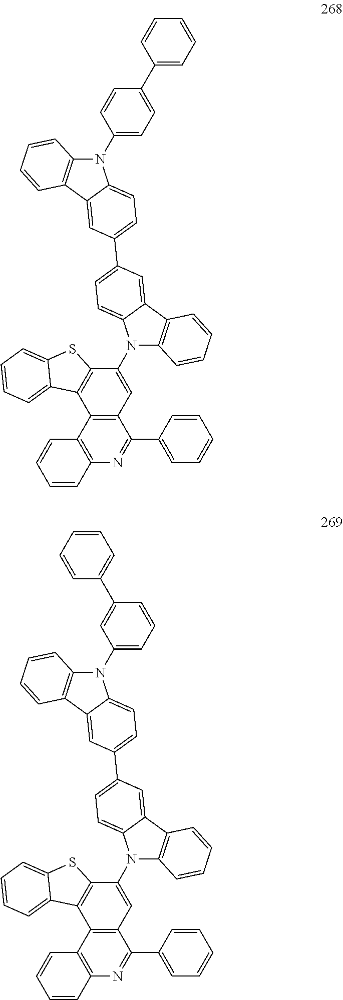 [SMILES: c1ccc(-c2ccc(-n3c4ccccc4c4cc(-c5ccc6c(c5)c5ccccc5n6-c5cc6c(-c7ccccc7)nc7ccccc7c6c6c5sc5ccccc56)ccc43)cc2)cc1 ; c1ccc(-c2cccc(-n3c4ccccc4c4cc(-c5ccc6c(c5)c5ccccc5n6-c5cc6c(-c7ccccc7)nc7ccccc7c6c6c5sc5ccccc56)ccc43)c2)cc1]

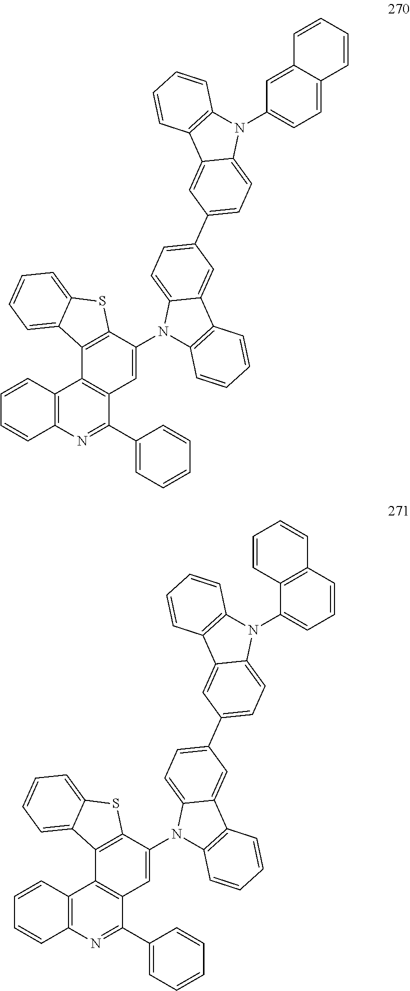 [SMILES: c1ccc(-c2nc3ccccc3c3c2cc(-n2c4ccccc4c4cc(-c5ccc6c(c5)c5ccccc5n6-c5ccc6ccccc6c5)ccc42)c2sc4ccccc4c23)cc1 ; c1ccc(-c2nc3ccccc3c3c2cc(-n2c4ccccc4c4cc(-c5ccc6c(c5)c5ccccc5n6-c5cccc6ccccc56)ccc42)c2sc4ccccc4c23)cc1]

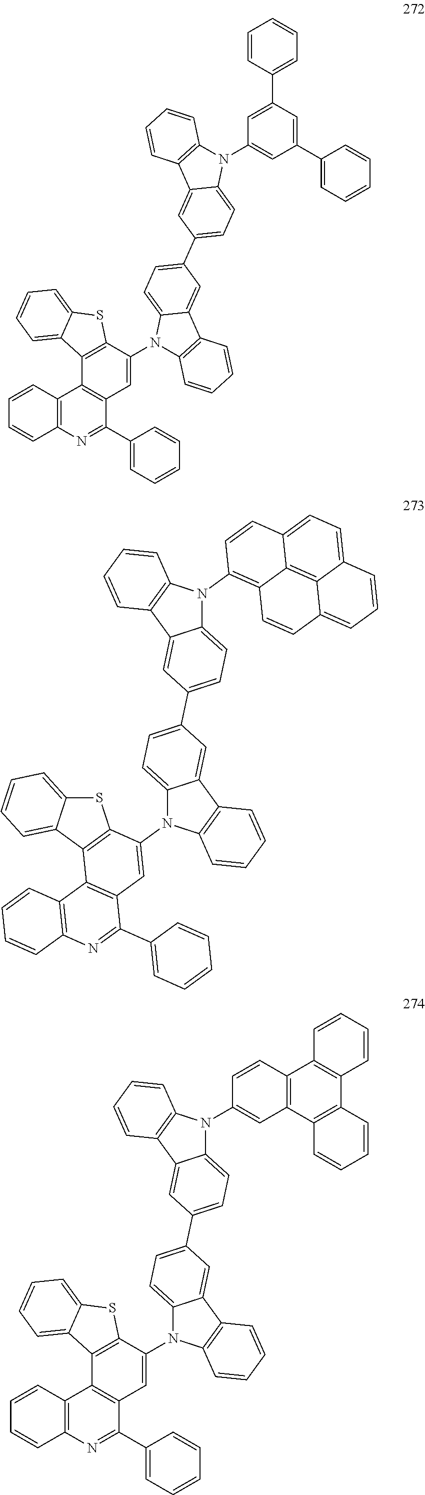 [SMILES: c1ccc(-c2cc(-c3ccccc3)cc(-n3c4ccccc4c4cc(-c5ccc6c(c5)c5ccccc5n6-c5cc6c(-c7ccccc7)nc7ccccc7c6c6c5sc5ccccc56)ccc43)c2)cc1 ; c1ccc(-c2nc3ccccc3c3c2cc(-n2c4ccccc4c4cc(-c5ccc6c(c5)c5ccccc5n6-c5ccc6ccc7cccc8ccc5c6c78)ccc42)c2sc4ccccc4c23)cc1 ; c1ccc(-c2nc3ccccc3c3c2cc(-n2c4ccccc4c4cc(-c5ccc6c(c5)c5ccccc5n6-c5ccc6c7ccccc7c7ccccc7c6c5)ccc42)c2sc4ccccc4c23)cc1]

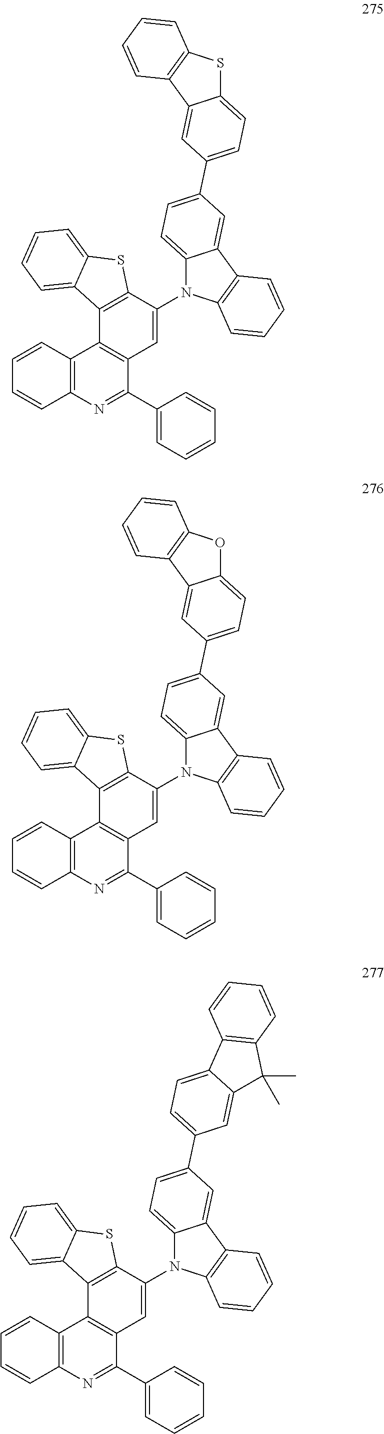

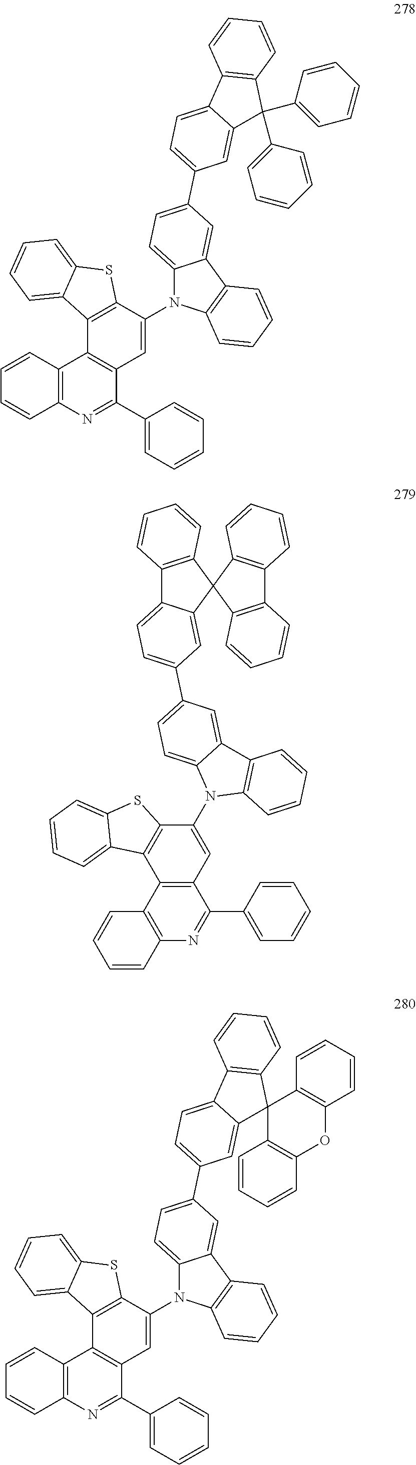

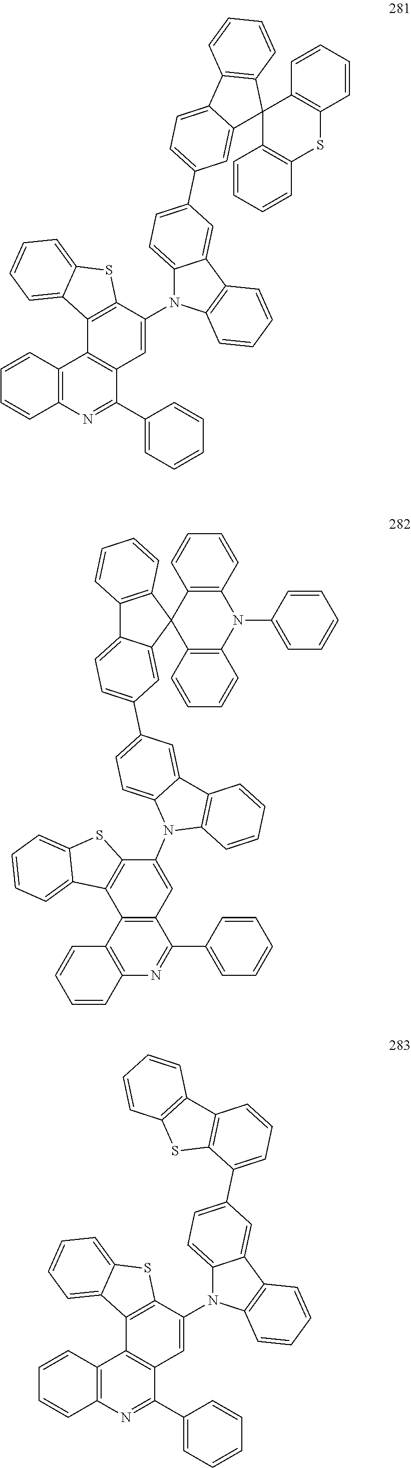 [SMILES: c1ccc(-c2nc3ccccc3c3c2cc(-n2c4ccccc4c4cc(-c5ccc6c(c5)C5(c7ccccc7Sc7ccccc75)c5ccccc5-6)ccc42)c2sc4ccccc4c23)cc1 ; c1ccc(-c2nc3ccccc3c3c2cc(-n2c4ccccc4c4cc(-c5ccc6c(c5)C5(c7ccccc7-6)c6ccccc6N(c6ccccc6)c6ccccc65)ccc42)c2sc4ccccc4c23)cc1 ; c1ccc(-c2nc3ccccc3c3c2cc(-n2c4ccccc4c4cc(-c5cccc6c5sc5ccccc56)ccc42)c2sc4ccccc4c23)cc1]

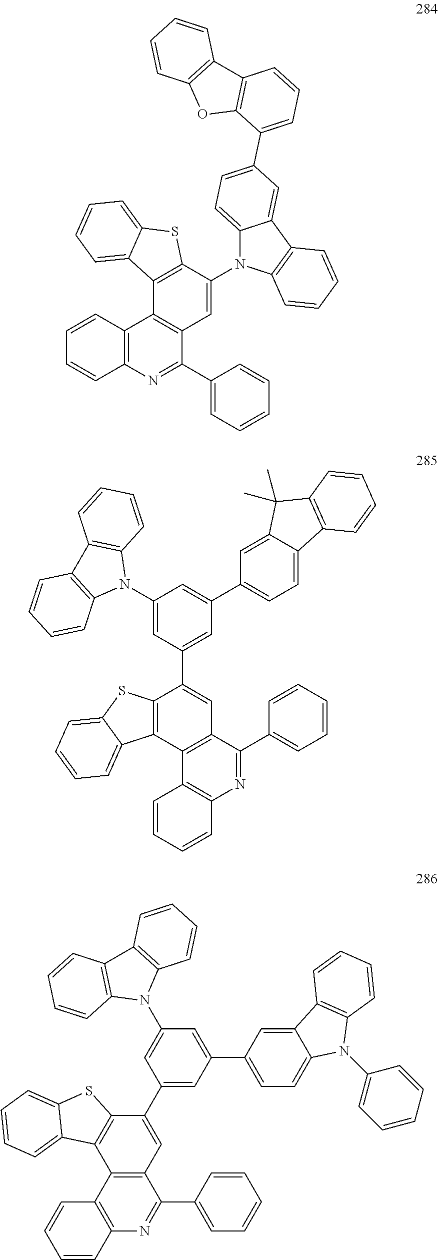 [SMILES: c1ccc(-c2nc3ccccc3c3c2cc(-n2c4ccccc4c4cc(-c5cccc6c5oc5ccccc56)ccc42)c2sc4ccccc4c23)cc1 ; CC1(C)c2ccccc2-c2ccc(-c3cc(-c4cc5c(-c6ccccc6)nc6ccccc6c5c5c4sc4ccccc45)cc(-n4c5ccccc5c5ccccc54)c3)cc21 ; c1ccc(-c2nc3ccccc3c3c2cc(-c2cc(-c4ccc5c(c4)c4ccccc4n5-c4ccccc4)cc(-n4c5ccccc5c5ccccc54)c2)c2sc4ccccc4c23)cc1]

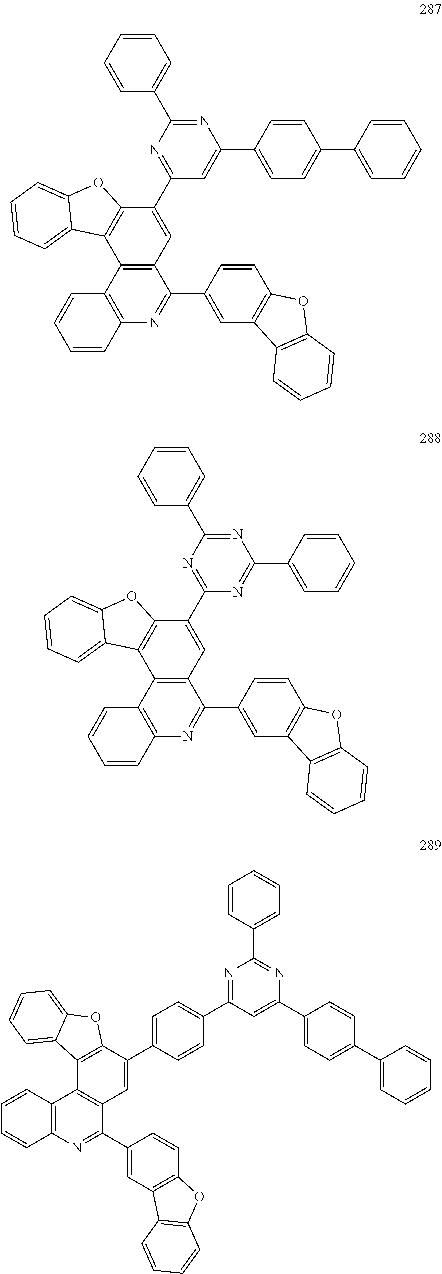 [SMILES: c1ccc(-c2ccc(-c3cc(-c4cc5c(-c6ccc7oc8ccccc8c7c6)nc6ccccc6c5c5c4oc4ccccc45)nc(-c4ccccc4)n3)cc2)cc1 ; c1ccc(-c2nc(-c3ccccc3)nc(-c3cc4c(-c5ccc6oc7ccccc7c6c5)nc5ccccc5c4c4c3oc3ccccc34)n2)cc1 ; c1ccc(-c2ccc(-c3cc(-c4ccc(-c5cc6c(-c7ccc8oc9ccccc9c8c7)nc7ccccc7c6c6c5oc5ccccc56)cc4)nc(-c4ccccc4)n3)cc2)cc1]

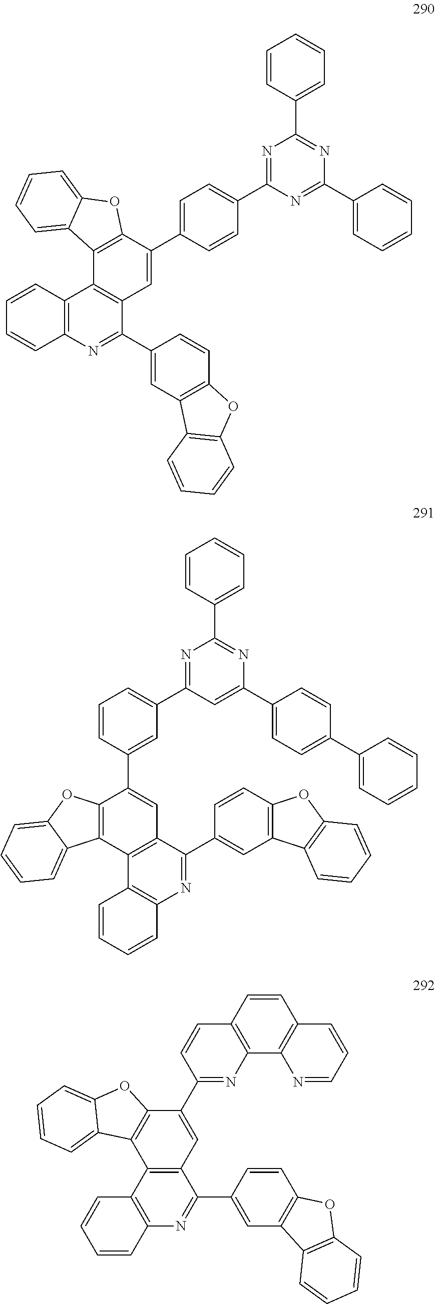

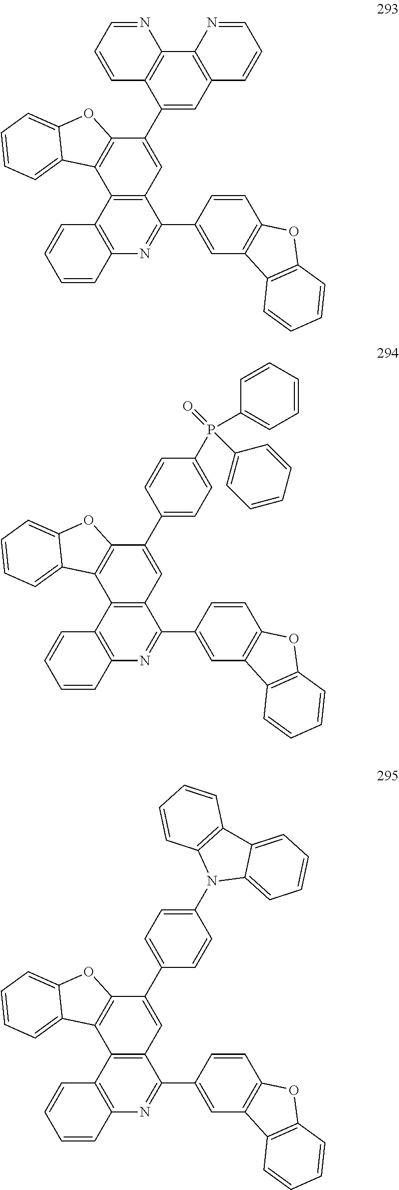

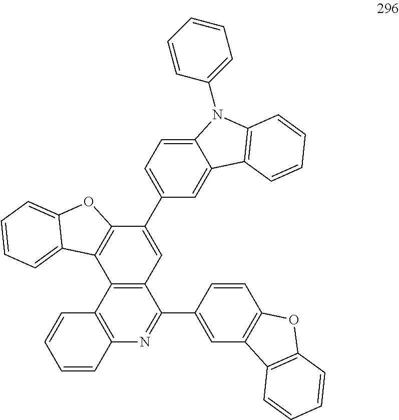 [SMILES: c1ccc(-n2c3ccccc3c3cc(-c4cc5c(-c6ccc7oc8ccccc8c7c6)nc6ccccc6c5c5c4oc4ccccc45)ccc32)cc1]

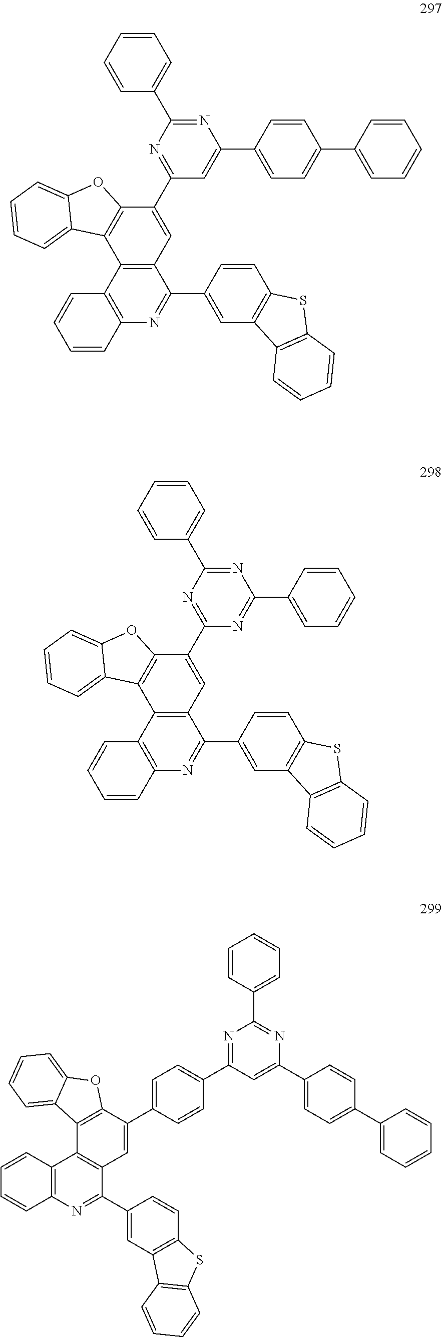 [SMILES: c1ccc(-c2ccc(-c3cc(-c4cc5c(-c6ccc7sc8ccccc8c7c6)nc6ccccc6c5c5c4oc4ccccc45)nc(-c4ccccc4)n3)cc2)cc1 ; c1ccc(-c2nc(-c3ccccc3)nc(-c3cc4c(-c5ccc6sc7ccccc7c6c5)nc5ccccc5c4c4c3oc3ccccc34)n2)cc1 ; c1ccc(-c2ccc(-c3cc(-c4ccc(-c5cc6c(-c7ccc8sc9ccccc9c8c7)nc7ccccc7c6c6c5oc5ccccc56)cc4)nc(-c4ccccc4)n3)cc2)cc1]

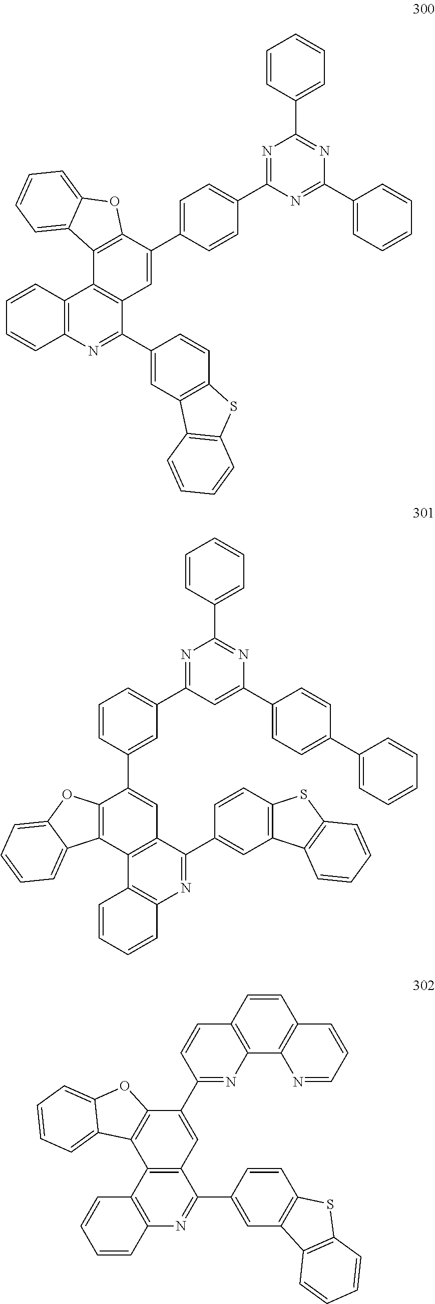

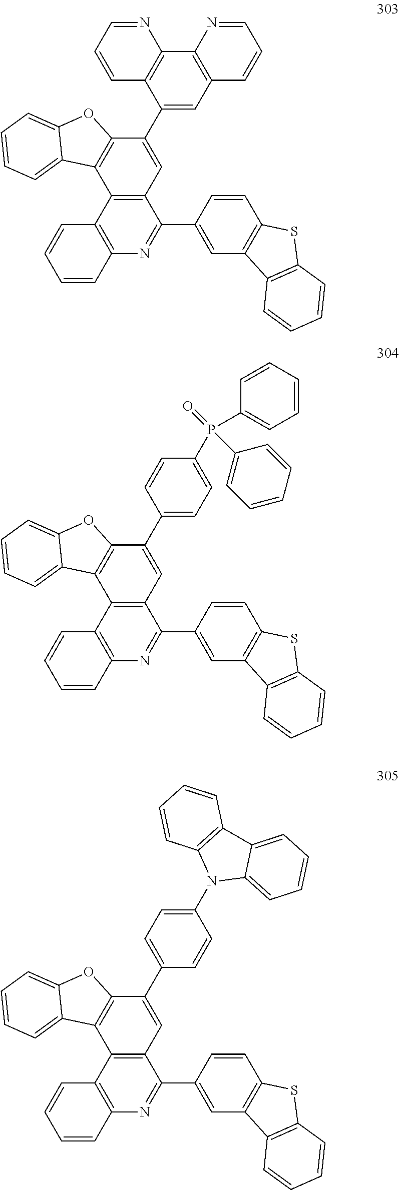

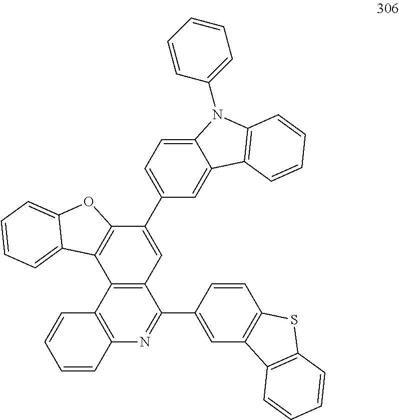 [SMILES: c1ccc(-n2c3ccccc3c3cc(-c4cc5c(-c6ccc7sc8ccccc8c7c6)nc6ccccc6c5c5c4oc4ccccc45)ccc32)cc1]

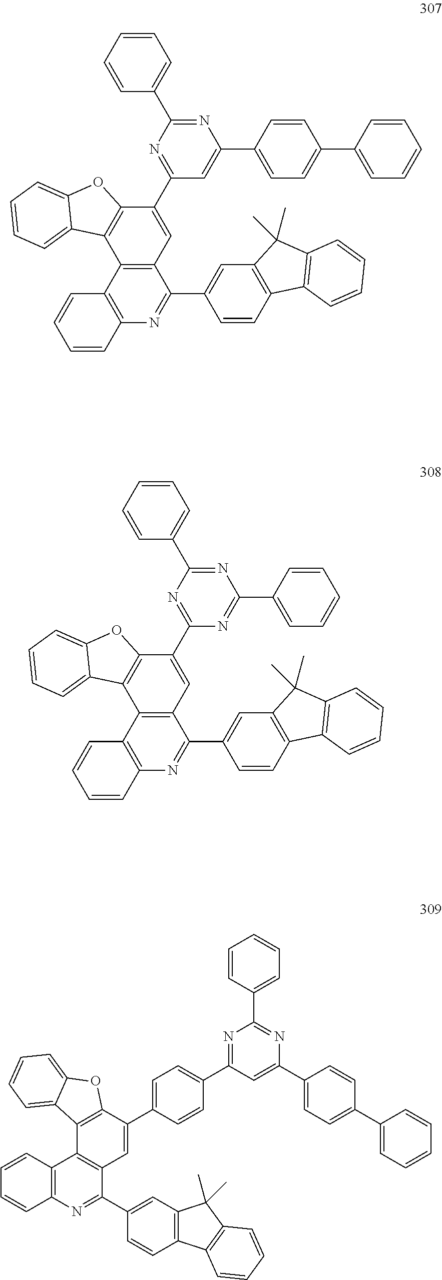 [SMILES: CC1(C)c2ccccc2-c2ccc(-c3nc4ccccc4c4c3cc(-c3cc(-c5ccc(-c6ccccc6)cc5)nc(-c5ccccc5)n3)c3oc5ccccc5c34)cc21 ; CC1(C)c2ccccc2-c2ccc(-c3nc4ccccc4c4c3cc(-c3nc(-c5ccccc5)nc(-c5ccccc5)n3)c3oc5ccccc5c34)cc21 ; CC1(C)c2ccccc2-c2ccc(-c3nc4ccccc4c4c3cc(-c3ccc(-c5cc(-c6ccc(-c7ccccc7)cc6)nc(-c6ccccc6)n5)cc3)c3oc5ccccc5c34)cc21]

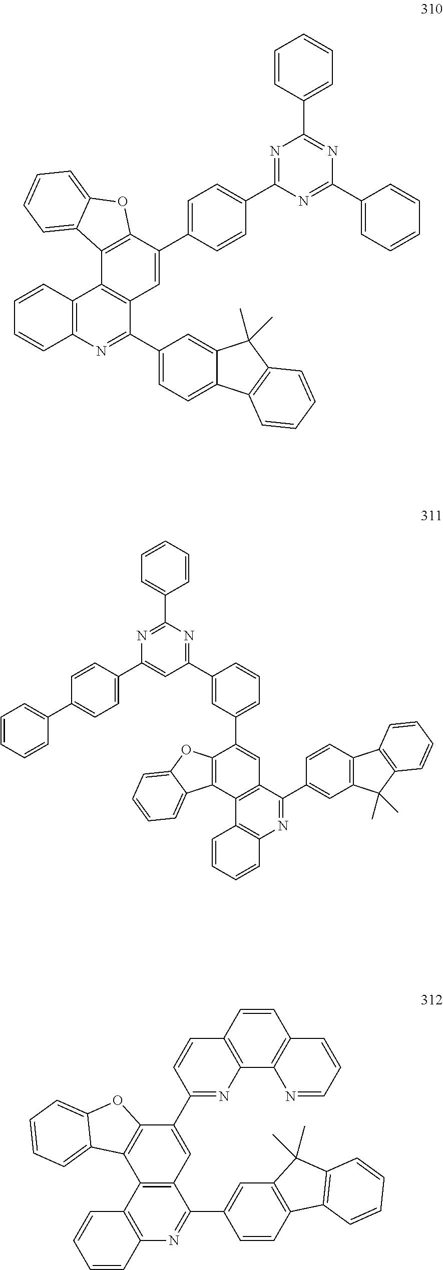 [SMILES: CC1(C)c2ccccc2-c2ccc(-c3nc4ccccc4c4c3cc(-c3ccc(-c5nc(-c6ccccc6)nc(-c6ccccc6)n5)cc3)c3oc5ccccc5c34)cc21 ; CC1(C)c2ccccc2-c2ccc(-c3nc4ccccc4c4c3cc(-c3cccc(-c5cc(-c6ccc(-c7ccccc7)cc6)nc(-c6ccccc6)n5)c3)c3oc5ccccc5c34)cc21 ; CC1(C)c2ccccc2-c2ccc(-c3nc4ccccc4c4c3cc(-c3ccc5ccc6cccnc6c5n3)c3oc5ccccc5c34)cc21]

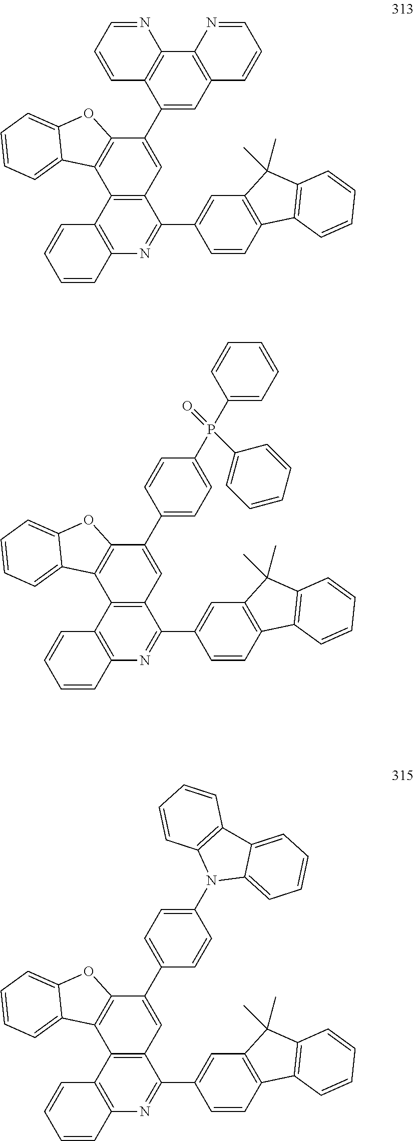 [SMILES: CC1(C)c2ccccc2-c2ccc(-c3nc4ccccc4c4c3cc(-c3cc5cccnc5c5ncccc35)c3oc5ccccc5c34)cc21 ; CC1(C)c2ccccc2-c2ccc(-c3nc4ccccc4c4c3cc(-c3ccc(P(=O)(c5ccccc5)c5ccccc5)cc3)c3oc5ccccc5c34)cc21 ; CC1(C)c2ccccc2-c2ccc(-c3nc4ccccc4c4c3cc(-c3ccc(-n5c6ccccc6c6ccccc65)cc3)c3oc5ccccc5c34)cc21]

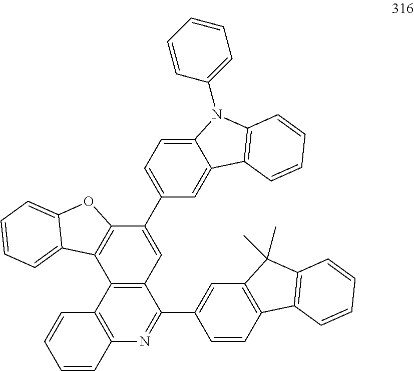 [SMILES: CC1(C)c2ccccc2-c2ccc(-c3nc4ccccc4c4c3cc(-c3ccc5c(c3)c3ccccc3n5-c3ccccc3)c3oc5ccccc5c34)cc21]

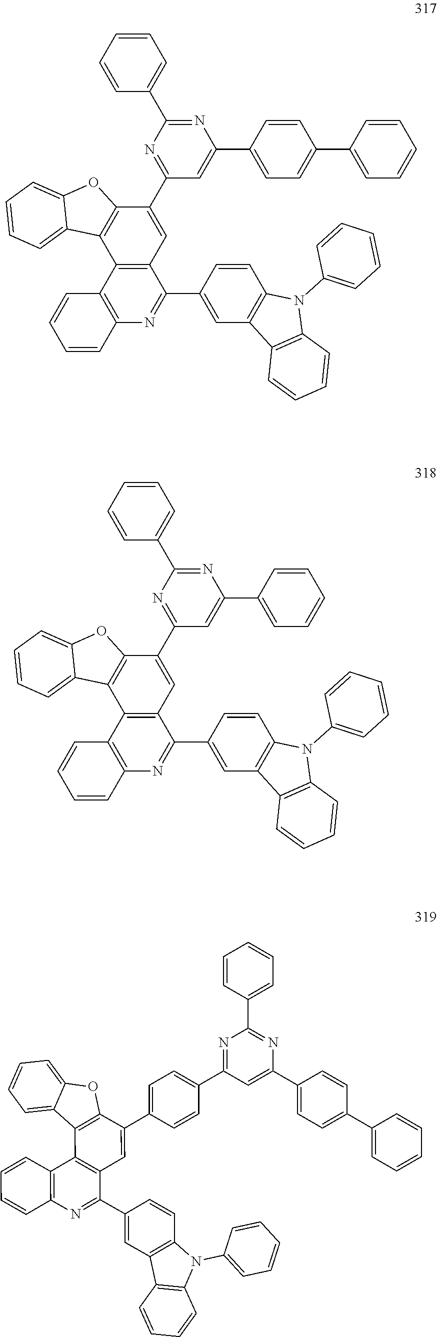 [SMILES: c1ccc(-c2ccc(-c3cc(-c4cc5c(-c6ccc7c(c6)c6ccccc6n7-c6ccccc6)nc6ccccc6c5c5c4oc4ccccc45)nc(-c4ccccc4)n3)cc2)cc1 ; c1ccc(-c2cc(-c3cc4c(-c5ccc6c(c5)c5ccccc5n6-c5ccccc5)nc5ccccc5c4c4c3oc3ccccc34)nc(-c3ccccc3)n2)cc1 ; c1ccc(-c2ccc(-c3cc(-c4ccc(-c5cc6c(-c7ccc8c(c7)c7ccccc7n8-c7ccccc7)nc7ccccc7c6c6c5oc5ccccc56)cc4)nc(-c4ccccc4)n3)cc2)cc1]

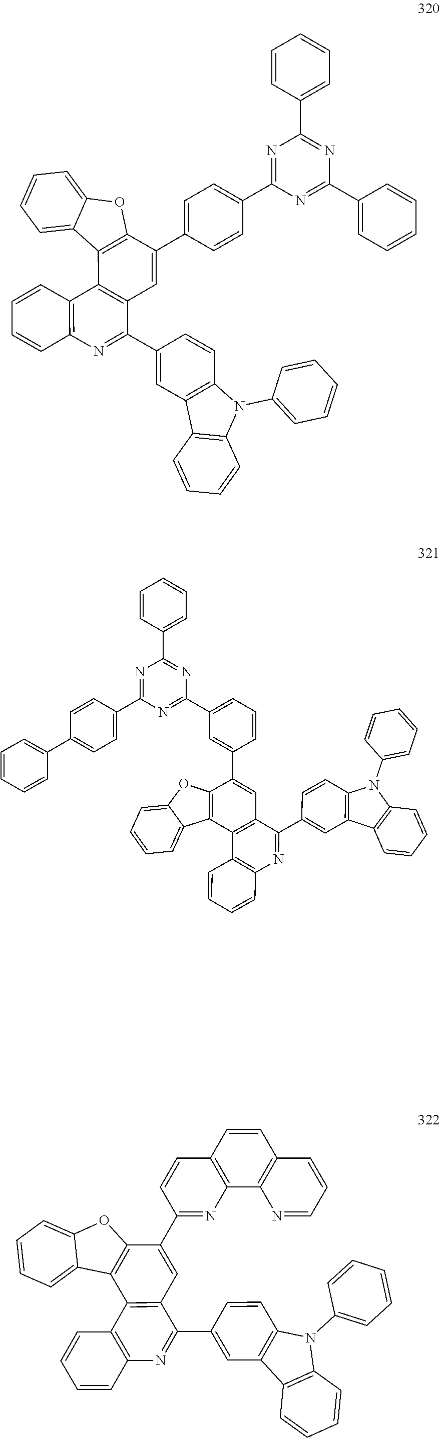 [SMILES: c1ccc(-c2nc(-c3ccccc3)nc(-c3ccc(-c4cc5c(-c6ccc7c(c6)c6ccccc6n7-c6ccccc6)nc6ccccc6c5c5c4oc4ccccc45)cc3)n2)cc1 ; c1ccc(-c2ccc(-c3nc(-c4ccccc4)nc(-c4cccc(-c5cc6c(-c7ccc8c(c7)c7ccccc7n8-c7ccccc7)nc7ccccc7c6c6c5oc5ccccc56)c4)n3)cc2)cc1 ; c1ccc(-n2c3ccccc3c3cc(-c4nc5ccccc5c5c4cc(-c4ccc6ccc7cccnc7c6n4)c4oc6ccccc6c45)ccc32)cc1]

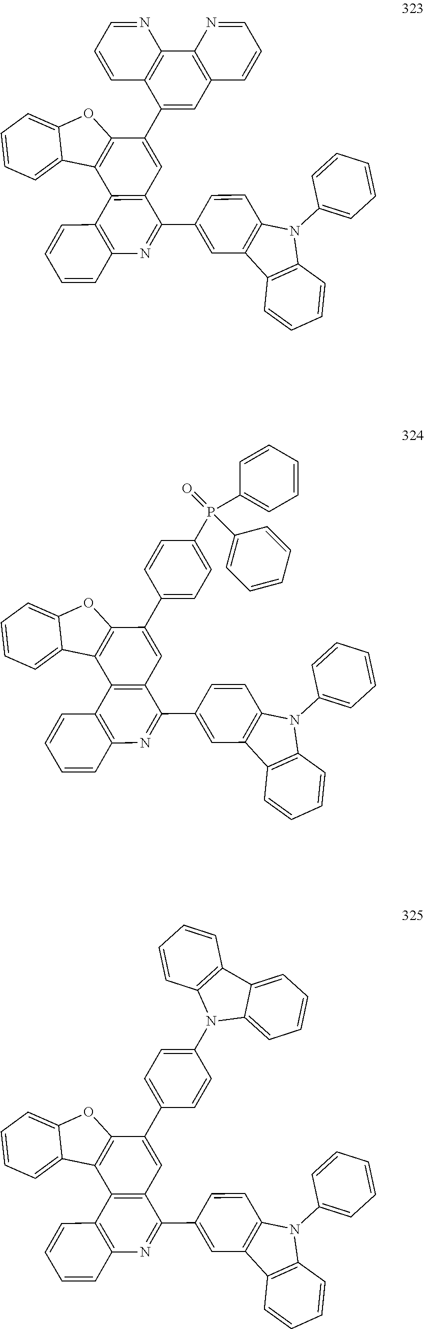 [SMILES: c1ccc(-n2c3ccccc3c3cc(-c4nc5ccccc5c5c4cc(-c4cc6cccnc6c6ncccc46)c4oc6ccccc6c45)ccc32)cc1 ; O=P(c1ccccc1)(c1ccccc1)c1ccc(-c2cc3c(-c4ccc5c(c4)c4ccccc4n5-c4ccccc4)nc4ccccc4c3c3c2oc2ccccc23)cc1 ; c1ccc(-n2c3ccccc3c3cc(-c4nc5ccccc5c5c4cc(-c4ccc(-n6c7ccccc7c7ccccc76)cc4)c4oc6ccccc6c45)ccc32)cc1]

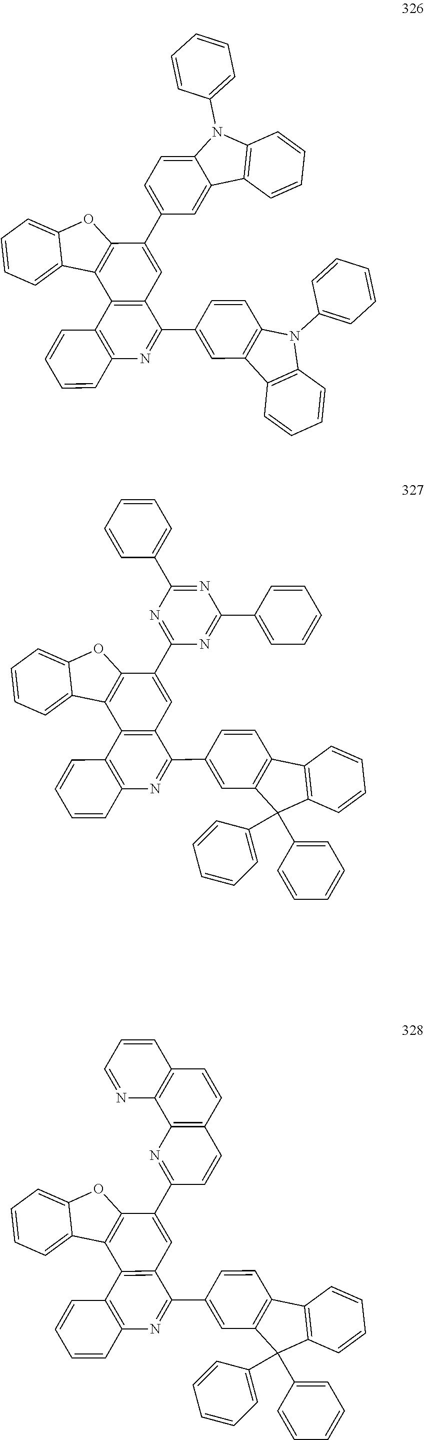 [SMILES: c1ccc(-n2c3ccccc3c3cc(-c4nc5ccccc5c5c4cc(-c4ccc6c(c4)c4ccccc4n6-c4ccccc4)c4oc6ccccc6c45)ccc32)cc1 ; c1ccc(-c2nc(-c3ccccc3)nc(-c3cc4c(-c5ccc6c(c5)C(c5ccccc5)(c5ccccc5)c5ccccc5-6)nc5ccccc5c4c4c3oc3ccccc34)n2)cc1 ; c1ccc(C2(c3ccccc3)c3ccccc3-c3ccc(-c4nc5ccccc5c5c4cc(-c4ccc6ccc7cccnc7c6n4)c4oc6ccccc6c45)cc32)cc1]

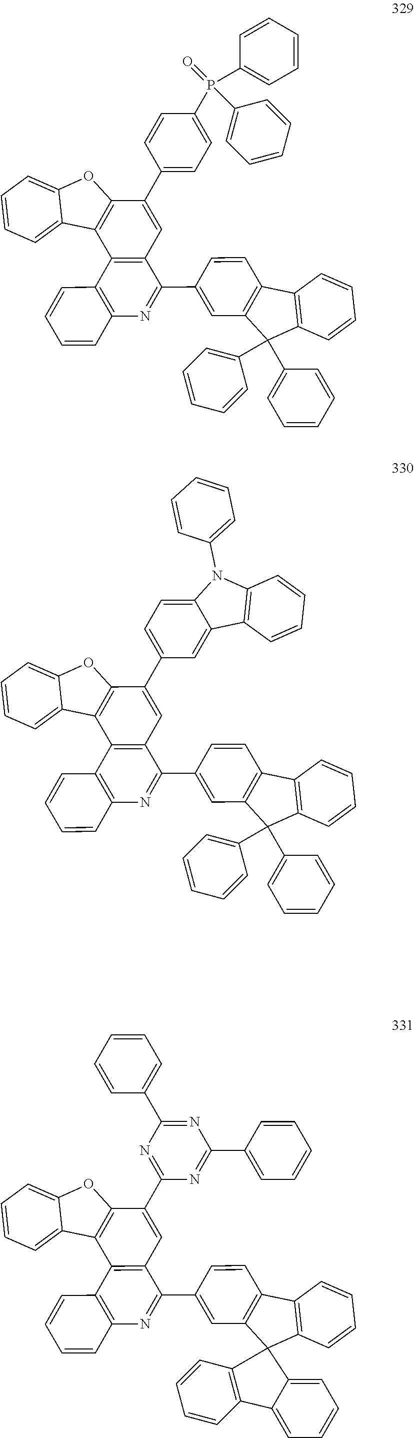 [SMILES: O=P(c1ccccc1)(c1ccccc1)c1ccc(-c2cc3c(-c4ccc5c(c4)C(c4ccccc4)(c4ccccc4)c4ccccc4-5)nc4ccccc4c3c3c2oc2ccccc23)cc1 ; c1ccc(-n2c3ccccc3c3cc(-c4cc5c(-c6ccc7c(c6)C(c6ccccc6)(c6ccccc6)c6ccccc6-7)nc6ccccc6c5c5c4oc4ccccc45)ccc32)cc1 ; c1ccc(-c2nc(-c3ccccc3)nc(-c3cc4c(-c5ccc6c(c5)C5(c7ccccc7-c7ccccc75)c5ccccc5-6)nc5ccccc5c4c4c3oc3ccccc34)n2)cc1]

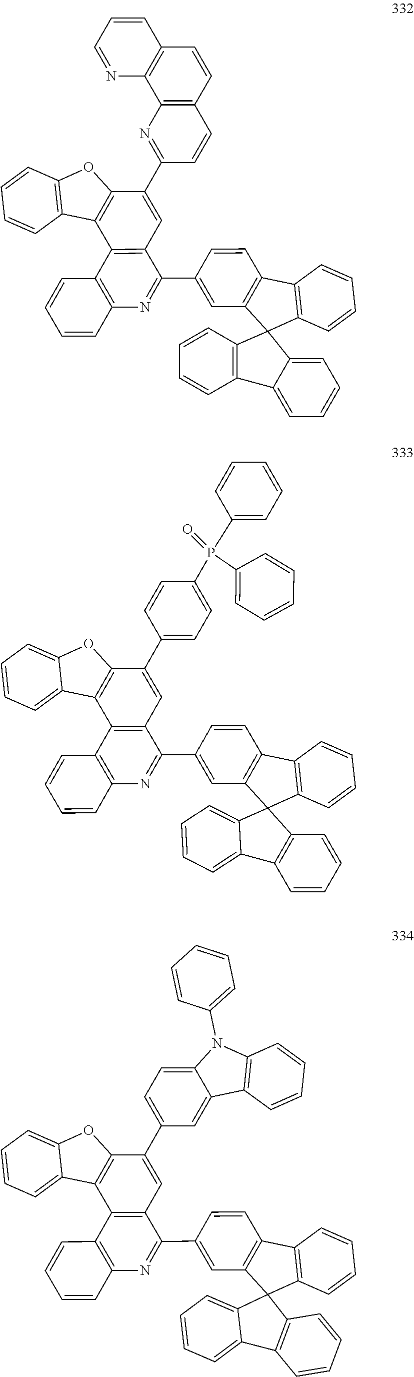 [SMILES: c1ccc2c(c1)-c1ccccc1C21c2ccccc2-c2ccc(-c3nc4ccccc4c4c3cc(-c3ccc5ccc6cccnc6c5n3)c3oc5ccccc5c34)cc21 ; O=P(c1ccccc1)(c1ccccc1)c1ccc(-c2cc3c(-c4ccc5c(c4)C4(c6ccccc6-c6ccccc64)c4ccccc4-5)nc4ccccc4c3c3c2oc2ccccc23)cc1 ; c1ccc(-n2c3ccccc3c3cc(-c4cc5c(-c6ccc7c(c6)C6(c8ccccc8-c8ccccc86)c6ccccc6-7)nc6ccccc6c5c5c4oc4ccccc45)ccc32)cc1]

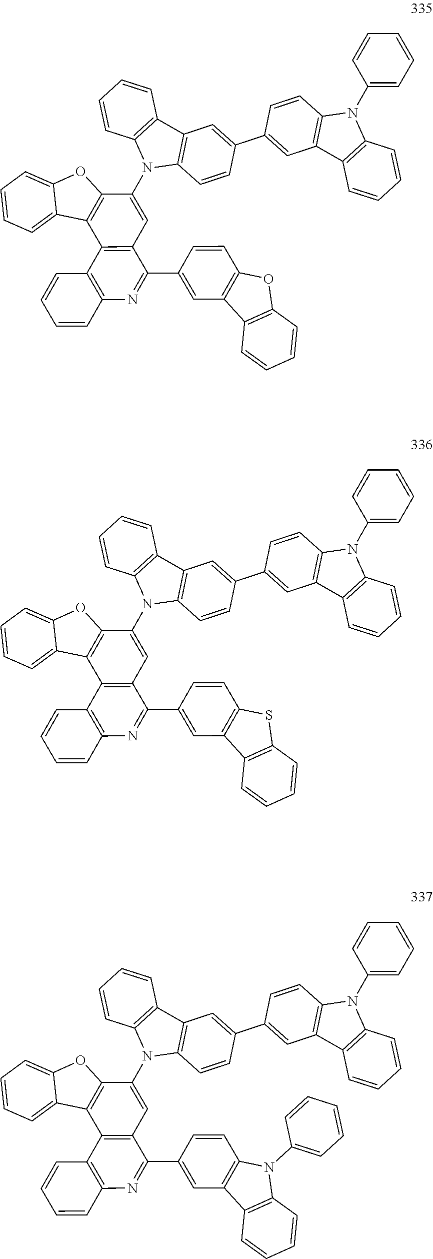 [SMILES: c1ccc(-n2c3ccccc3c3cc(-c4ccc5c(c4)c4ccccc4n5-c4cc5c(-c6ccc7oc8ccccc8c7c6)nc6ccccc6c5c5c4oc4ccccc45)ccc32)cc1 ; c1ccc(-n2c3ccccc3c3cc(-c4ccc5c(c4)c4ccccc4n5-c4cc5c(-c6ccc7sc8ccccc8c7c6)nc6ccccc6c5c5c4oc4ccccc45)ccc32)cc1 ; c1ccc(-n2c3ccccc3c3cc(-c4ccc5c(c4)c4ccccc4n5-c4cc5c(-c6ccc7c(c6)c6ccccc6n7-c6ccccc6)nc6ccccc6c5c5c4oc4ccccc45)ccc32)cc1]

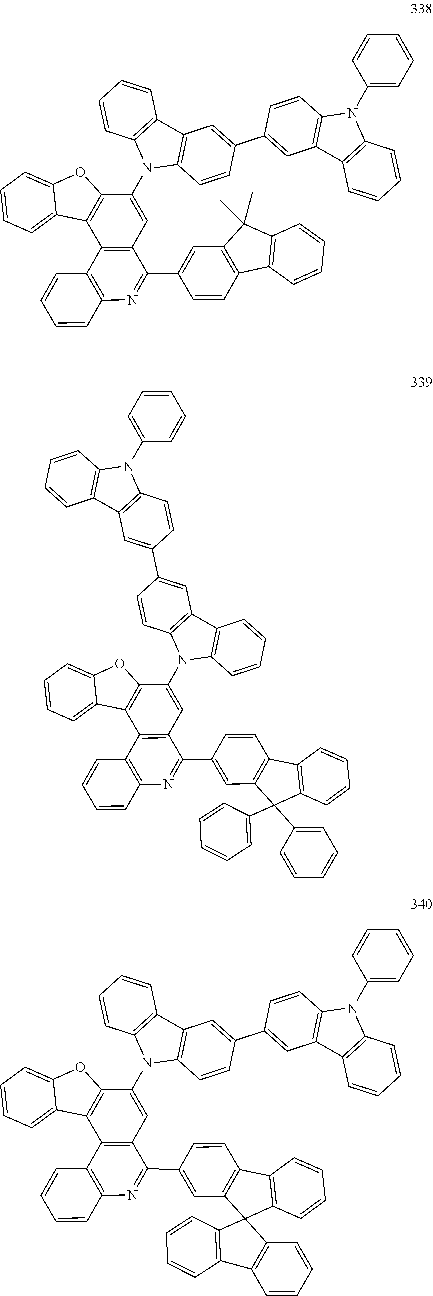 [SMILES: CC1(C)c2ccccc2-c2ccc(-c3nc4ccccc4c4c3cc(-n3c5ccccc5c5cc(-c6ccc7c(c6)c6ccccc6n7-c6ccccc6)ccc53)c3oc5ccccc5c34)cc21 ; c1ccc(-n2c3ccccc3c3cc(-c4ccc5c(c4)c4ccccc4n5-c4cc5c(-c6ccc7c(c6)C(c6ccccc6)(c6ccccc6)c6ccccc6-7)nc6ccccc6c5c5c4oc4ccccc45)ccc32)cc1 ; c1ccc(-n2c3ccccc3c3cc(-c4ccc5c(c4)c4ccccc4n5-c4cc5c(-c6ccc7c(c6)C6(c8ccccc8-c8ccccc86)c6ccccc6-7)nc6ccccc6c5c5c4oc4ccccc45)ccc32)cc1]

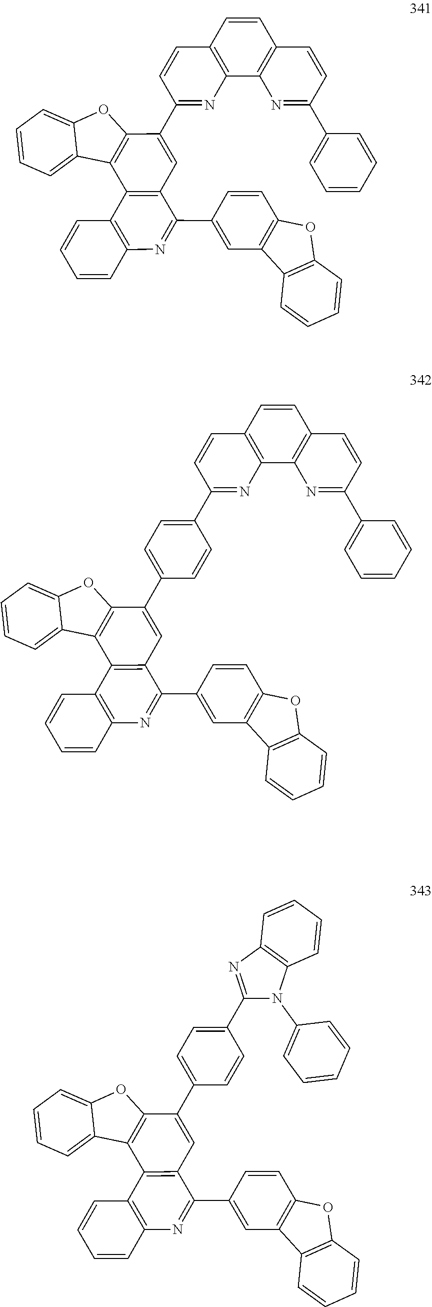 [SMILES: c1ccc(-c2ccc3ccc4ccc(-c5cc6c(-c7ccc8oc9ccccc9c8c7)nc7ccccc7c6c6c5oc5ccccc56)nc4c3n2)cc1 ; c1ccc(-c2ccc3ccc4ccc(-c5ccc(-c6cc7c(-c8ccc9oc%10ccccc%10c9c8)nc8ccccc8c7c7c6oc6ccccc67)cc5)nc4c3n2)cc1 ; c1ccc(-n2c(-c3ccc(-c4cc5c(-c6ccc7oc8ccccc8c7c6)nc6ccccc6c5c5c4oc4ccccc45)cc3)nc3ccccc32)cc1]

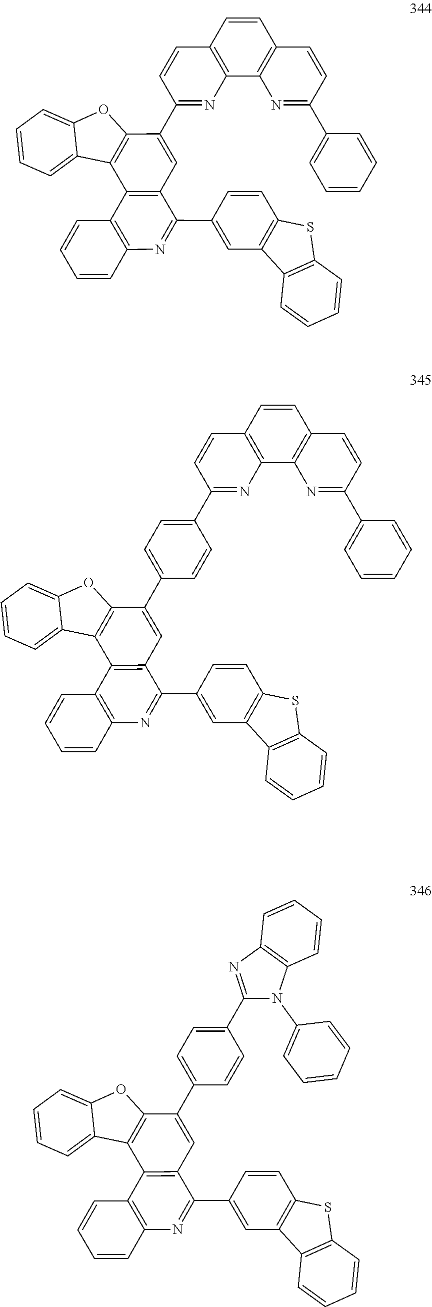 [SMILES: c1ccc(-c2ccc3ccc4ccc(-c5cc6c(-c7ccc8sc9ccccc9c8c7)nc7ccccc7c6c6c5oc5ccccc56)nc4c3n2)cc1 ; c1ccc(-c2ccc3ccc4ccc(-c5ccc(-c6cc7c(-c8ccc9sc%10ccccc%10c9c8)nc8ccccc8c7c7c6oc6ccccc67)cc5)nc4c3n2)cc1 ; c1ccc(-n2c(-c3ccc(-c4cc5c(-c6ccc7sc8ccccc8c7c6)nc6ccccc6c5c5c4oc4ccccc45)cc3)nc3ccccc32)cc1]

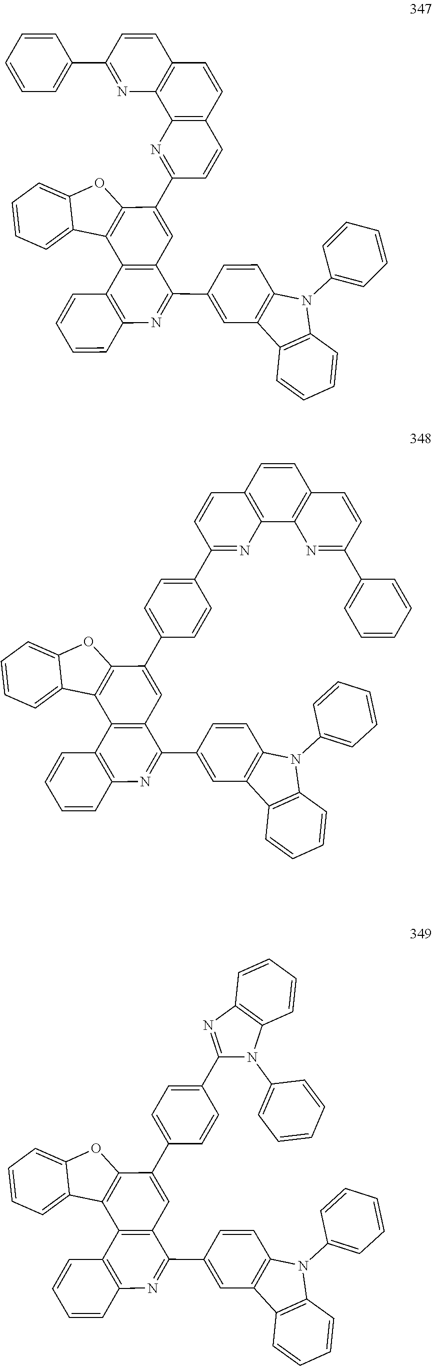 [SMILES: c1ccc(-c2ccc3ccc4ccc(-c5cc6c(-c7ccc8c(c7)c7ccccc7n8-c7ccccc7)nc7ccccc7c6c6c5oc5ccccc56)nc4c3n2)cc1 ; c1ccc(-c2ccc3ccc4ccc(-c5ccc(-c6cc7c(-c8ccc9c(c8)c8ccccc8n9-c8ccccc8)nc8ccccc8c7c7c6oc6ccccc67)cc5)nc4c3n2)cc1 ; c1ccc(-n2c(-c3ccc(-c4cc5c(-c6ccc7c(c6)c6ccccc6n7-c6ccccc6)nc6ccccc6c5c5c4oc4ccccc45)cc3)nc3ccccc32)cc1]

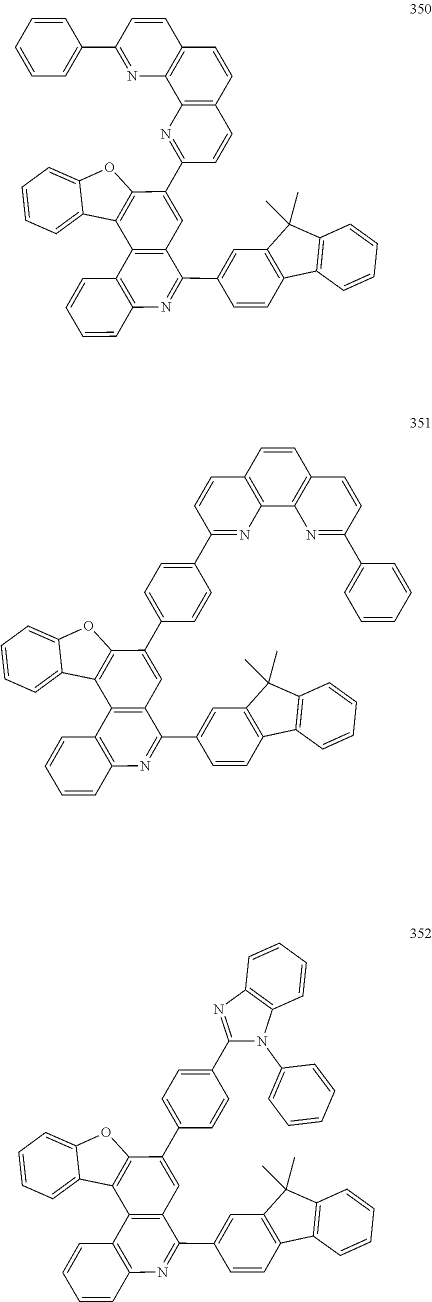 [SMILES: CC1(C)c2ccccc2-c2ccc(-c3nc4ccccc4c4c3cc(-c3ccc5ccc6ccc(-c7ccccc7)nc6c5n3)c3oc5ccccc5c34)cc21 ; CC1(C)c2ccccc2-c2ccc(-c3nc4ccccc4c4c3cc(-c3ccc(-c5ccc6ccc7ccc(-c8ccccc8)nc7c6n5)cc3)c3oc5ccccc5c34)cc21 ; CC1(C)c2ccccc2-c2ccc(-c3nc4ccccc4c4c3cc(-c3ccc(-c5nc6ccccc6n5-c5ccccc5)cc3)c3oc5ccccc5c34)cc21]

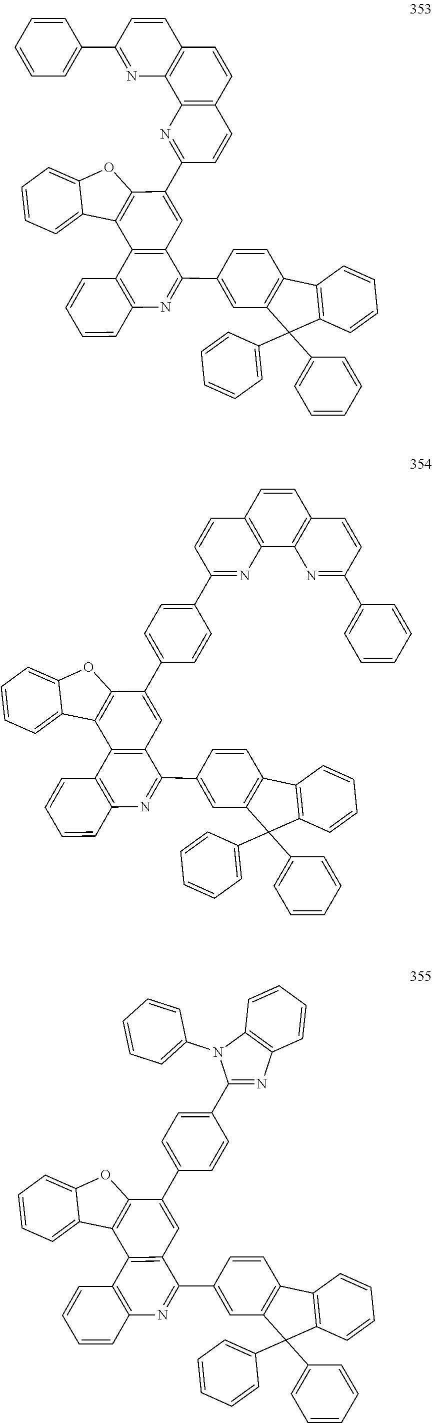 [SMILES: c1ccc(-c2ccc3ccc4ccc(-c5cc6c(-c7ccc8c(c7)C(c7ccccc7)(c7ccccc7)c7ccccc7-8)nc7ccccc7c6c6c5oc5ccccc56)nc4c3n2)cc1 ; c1ccc(-c2ccc3ccc4ccc(-c5ccc(-c6cc7c(-c8ccc9c(c8)C(c8ccccc8)(c8ccccc8)c8ccccc8-9)nc8ccccc8c7c7c6oc6ccccc67)cc5)nc4c3n2)cc1 ; c1ccc(-n2c(-c3ccc(-c4cc5c(-c6ccc7c(c6)C(c6ccccc6)(c6ccccc6)c6ccccc6-7)nc6ccccc6c5c5c4oc4ccccc45)cc3)nc3ccccc32)cc1]

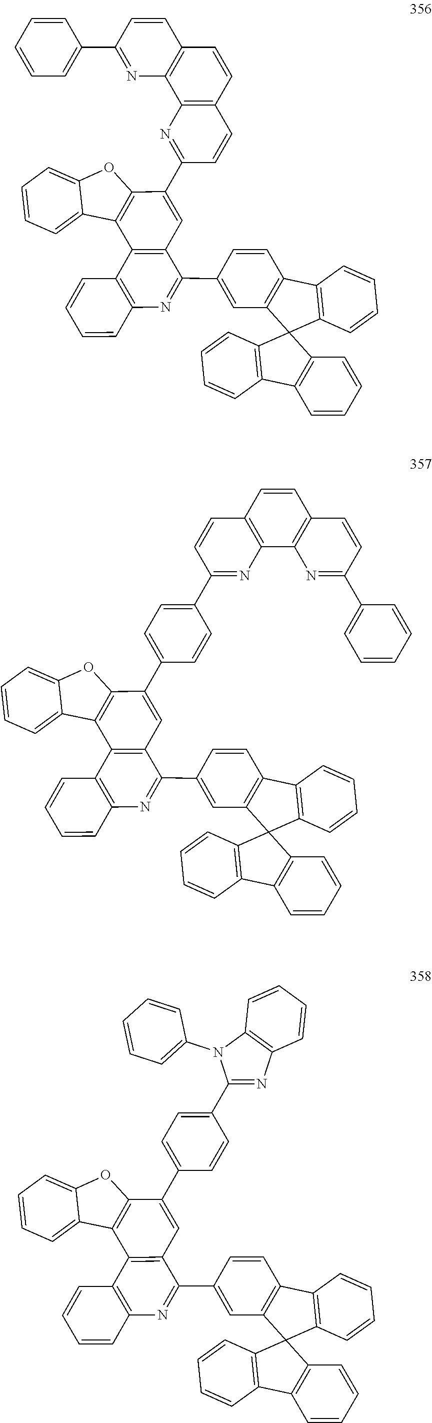 [SMILES: c1ccc(-c2ccc3ccc4ccc(-c5cc6c(-c7ccc8c(c7)C7(c9ccccc9-c9ccccc97)c7ccccc7-8)nc7ccccc7c6c6c5oc5ccccc56)nc4c3n2)cc1 ; c1ccc(-c2ccc3ccc4ccc(-c5ccc(-c6cc7c(-c8ccc9c(c8)C8(c%10ccccc%10-c%10ccccc%108)c8ccccc8-9)nc8ccccc8c7c7c6oc6ccccc67)cc5)nc4c3n2)cc1 ; c1ccc(-n2c(-c3ccc(-c4cc5c(-c6ccc7c(c6)C6(c8ccccc8-c8ccccc86)c6ccccc6-7)nc6ccccc6c5c5c4oc4ccccc45)cc3)nc3ccccc32)cc1]

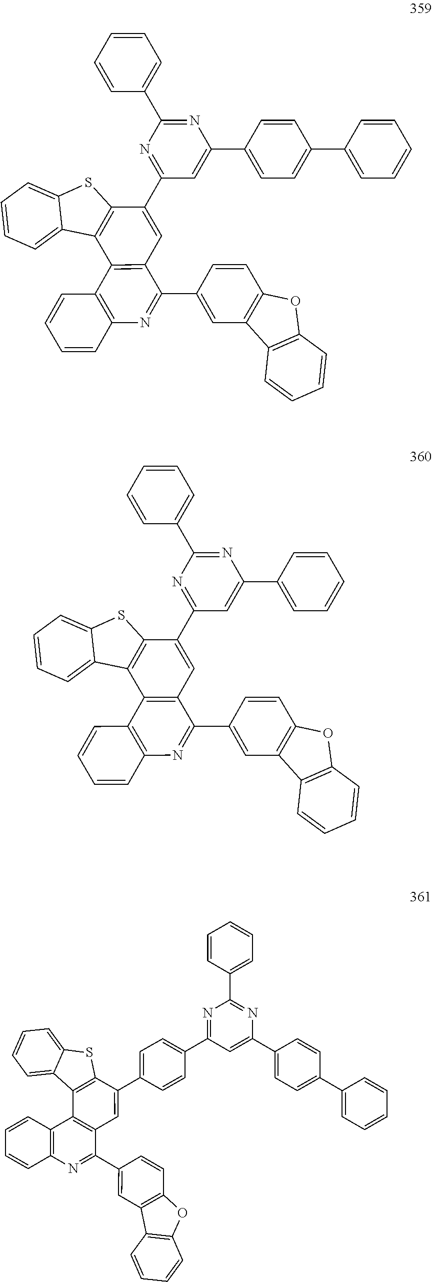 [SMILES: c1ccc(-c2ccc(-c3cc(-c4cc5c(-c6ccc7oc8ccccc8c7c6)nc6ccccc6c5c5c4sc4ccccc45)nc(-c4ccccc4)n3)cc2)cc1 ; c1ccc(-c2cc(-c3cc4c(-c5ccc6oc7ccccc7c6c5)nc5ccccc5c4c4c3sc3ccccc34)nc(-c3ccccc3)n2)cc1 ; c1ccc(-c2ccc(-c3cc(-c4ccc(-c5cc6c(-c7ccc8oc9ccccc9c8c7)nc7ccccc7c6c6c5sc5ccccc56)cc4)nc(-c4ccccc4)n3)cc2)cc1]

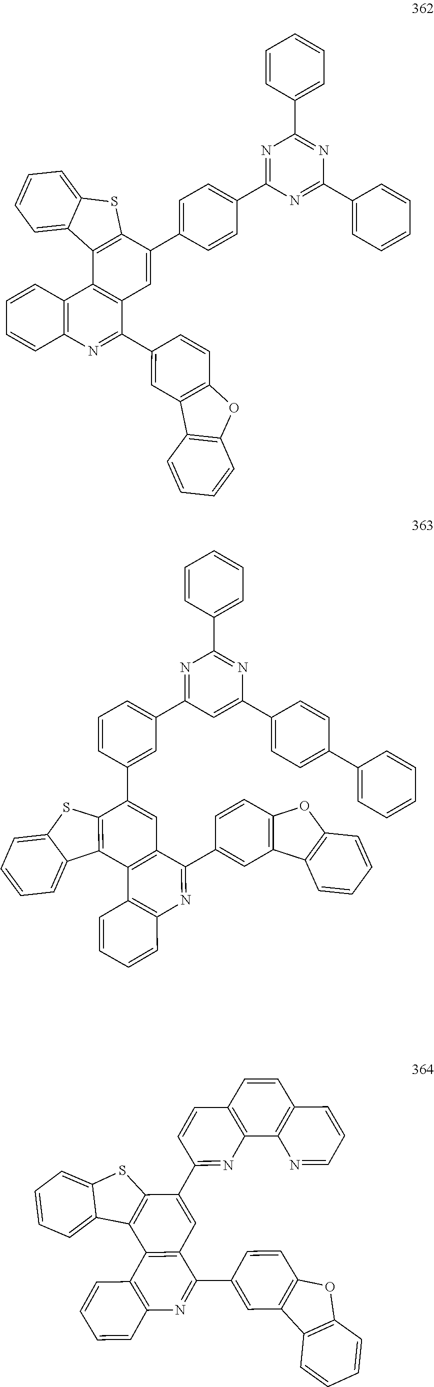 [SMILES: c1ccc(-c2nc(-c3ccccc3)nc(-c3ccc(-c4cc5c(-c6ccc7oc8ccccc8c7c6)nc6ccccc6c5c5c4sc4ccccc45)cc3)n2)cc1 ; c1ccc(-c2ccc(-c3cc(-c4cccc(-c5cc6c(-c7ccc8oc9ccccc9c8c7)nc7ccccc7c6c6c5sc5ccccc56)c4)nc(-c4ccccc4)n3)cc2)cc1 ; c1cnc2c(c1)ccc1ccc(-c3cc4c(-c5ccc6oc7ccccc7c6c5)nc5ccccc5c4c4c3sc3ccccc34)nc12]

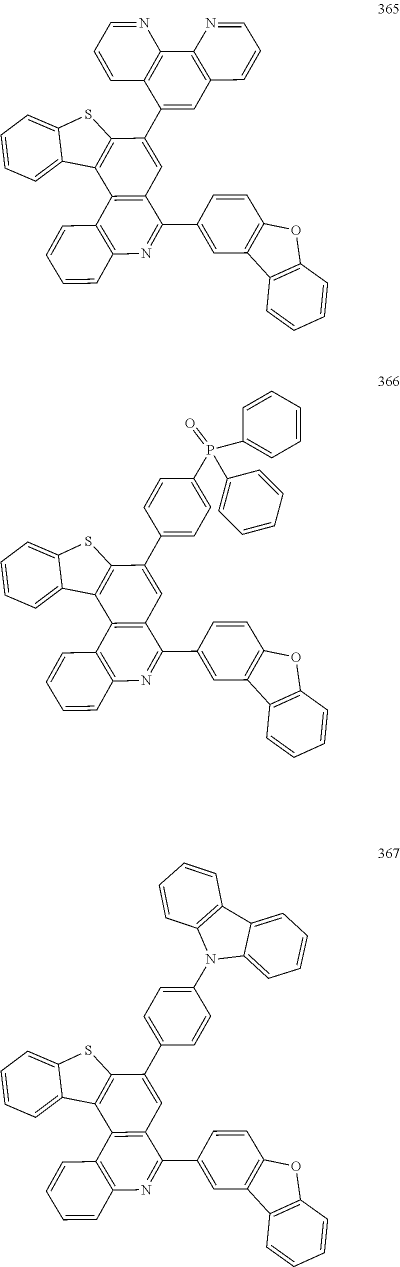 [SMILES: c1cnc2c(c1)cc(-c1cc3c(-c4ccc5oc6ccccc6c5c4)nc4ccccc4c3c3c1sc1ccccc13)c1cccnc12 ; O=P(c1ccccc1)(c1ccccc1)c1ccc(-c2cc3c(-c4ccc5oc6ccccc6c5c4)nc4ccccc4c3c3c2sc2ccccc23)cc1 ; c1ccc2c(c1)nc(-c1ccc3oc4ccccc4c3c1)c1cc(-c3ccc(-n4c5ccccc5c5ccccc54)cc3)c3sc4ccccc4c3c12]

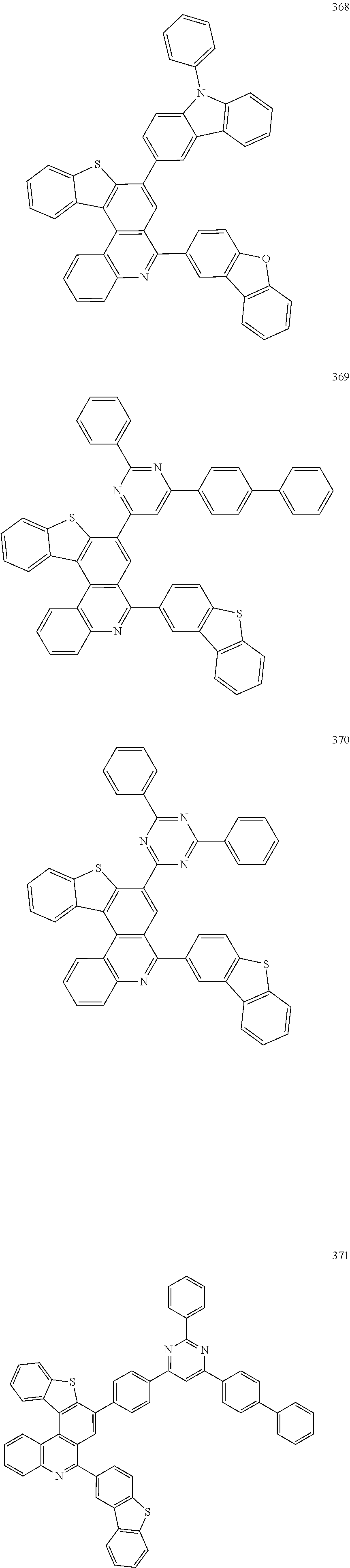 [SMILES: c1ccc(-n2c3ccccc3c3cc(-c4cc5c(-c6ccc7oc8ccccc8c7c6)nc6ccccc6c5c5c4sc4ccccc45)ccc32)cc1 ; c1ccc(-c2ccc(-c3cc(-c4cc5c(-c6ccc7sc8ccccc8c7c6)nc6ccccc6c5c5c4sc4ccccc45)nc(-c4ccccc4)n3)cc2)cc1 ; c1ccc(-c2nc(-c3ccccc3)nc(-c3cc4c(-c5ccc6sc7ccccc7c6c5)nc5ccccc5c4c4c3sc3ccccc34)n2)cc1 ; c1ccc(-c2ccc(-c3cc(-c4ccc(-c5cc6c(-c7ccc8sc9ccccc9c8c7)nc7ccccc7c6c6c5sc5ccccc56)cc4)nc(-c4ccccc4)n3)cc2)cc1]

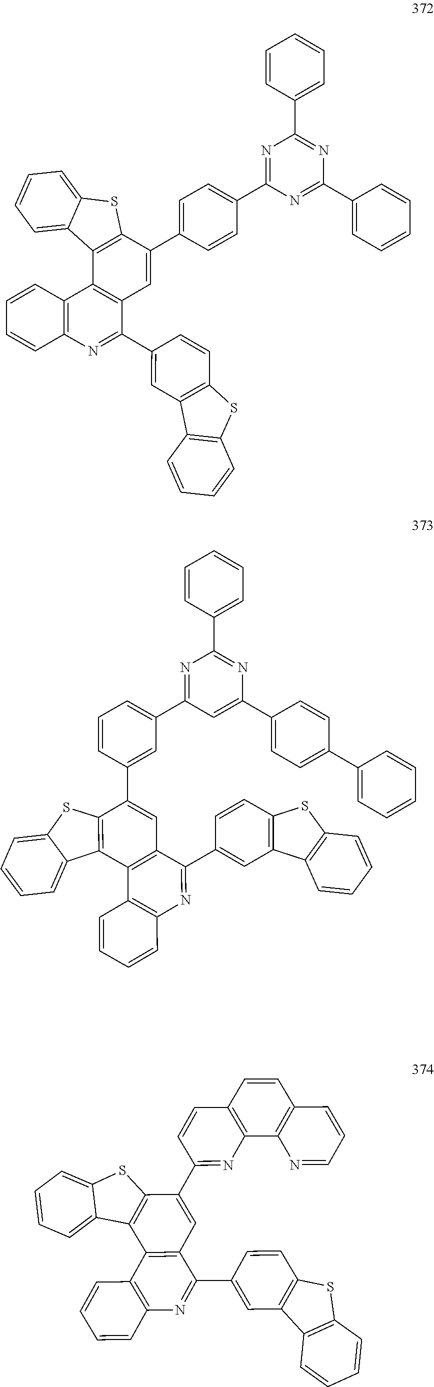 [SMILES: c1ccc(-c2nc(-c3ccccc3)nc(-c3ccc(-c4cc5c(-c6ccc7sc8ccccc8c7c6)nc6ccccc6c5c5c4sc4ccccc45)cc3)n2)cc1 ; c1ccc(-c2ccc(-c3cc(-c4cccc(-c5cc6c(-c7ccc8sc9ccccc9c8c7)nc7ccccc7c6c6c5sc5ccccc56)c4)nc(-c4ccccc4)n3)cc2)cc1 ; c1cnc2c(c1)ccc1ccc(-c3cc4c(-c5ccc6sc7ccccc7c6c5)nc5ccccc5c4c4c3sc3ccccc34)nc12]

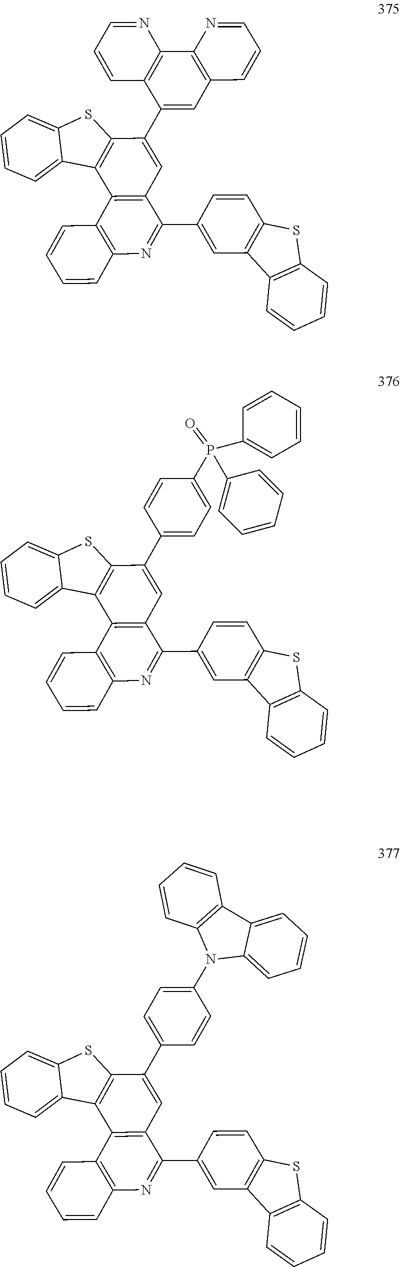 [SMILES: c1cnc2c(c1)cc(-c1cc3c(-c4ccc5sc6ccccc6c5c4)nc4ccccc4c3c3c1sc1ccccc13)c1cccnc12 ; O=P(c1ccccc1)(c1ccccc1)c1ccc(-c2cc3c(-c4ccc5sc6ccccc6c5c4)nc4ccccc4c3c3c2sc2ccccc23)cc1 ; c1ccc2c(c1)nc(-c1ccc3sc4ccccc4c3c1)c1cc(-c3ccc(-n4c5ccccc5c5ccccc54)cc3)c3sc4ccccc4c3c12]

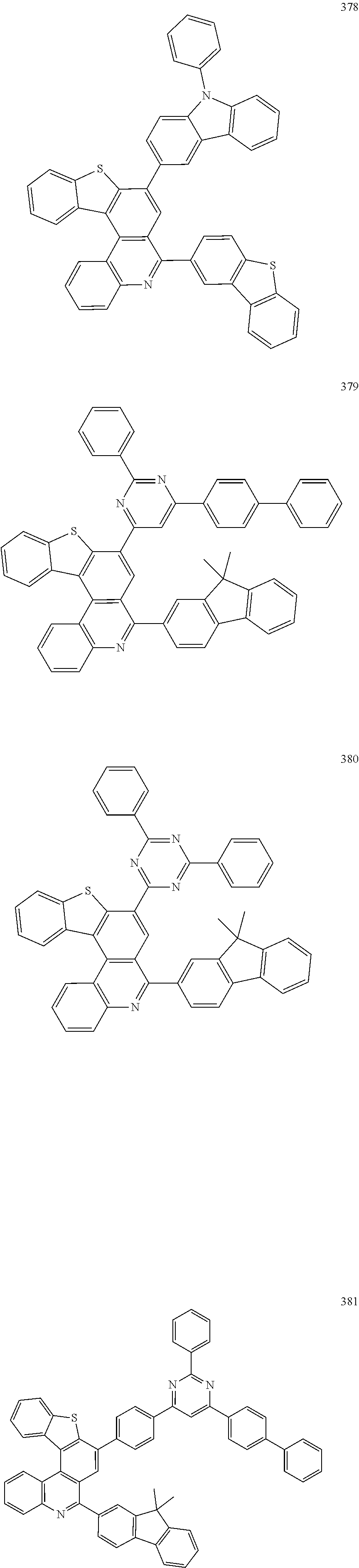 [SMILES: c1ccc(-n2c3ccccc3c3cc(-c4cc5c(-c6ccc7sc8ccccc8c7c6)nc6ccccc6c5c5c4sc4ccccc45)ccc32)cc1 ; CC1(C)c2ccccc2-c2ccc(-c3nc4ccccc4c4c3cc(-c3cc(-c5ccc(-c6ccccc6)cc5)nc(-c5ccccc5)n3)c3sc5ccccc5c34)cc21 ; CC1(C)c2ccccc2-c2ccc(-c3nc4ccccc4c4c3cc(-c3nc(-c5ccccc5)nc(-c5ccccc5)n3)c3sc5ccccc5c34)cc21 ; CC1(C)c2ccccc2-c2ccc(-c3nc4ccccc4c4c3cc(-c3ccc(-c5cc(-c6ccc(-c7ccccc7)cc6)nc(-c6ccccc6)n5)cc3)c3sc5ccccc5c34)cc21]

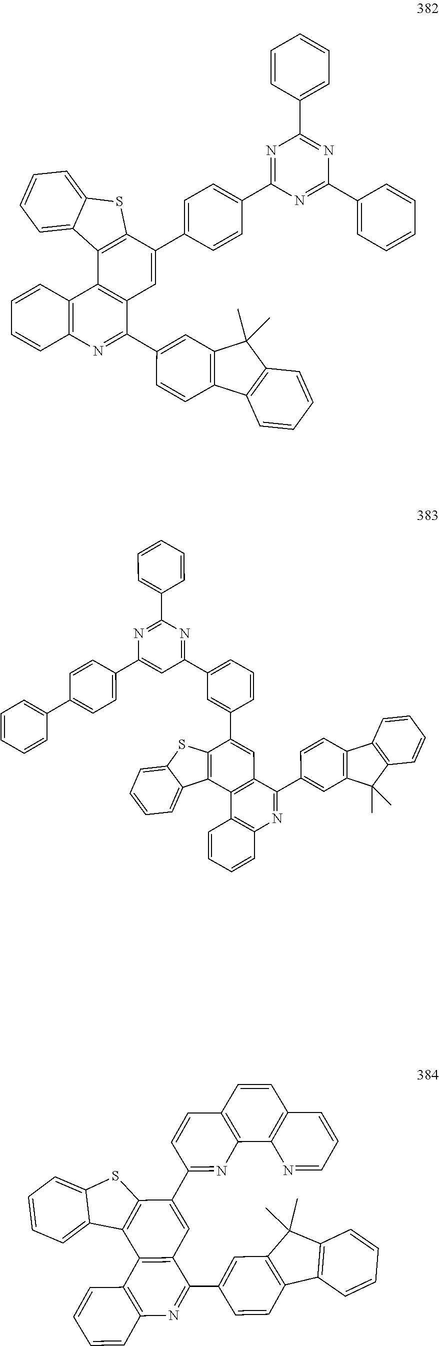 [SMILES: CC1(C)c2ccccc2-c2ccc(-c3nc4ccccc4c4c3cc(-c3ccc(-c5nc(-c6ccccc6)nc(-c6ccccc6)n5)cc3)c3sc5ccccc5c34)cc21 ; CC1(C)c2ccccc2-c2ccc(-c3nc4ccccc4c4c3cc(-c3cccc(-c5cc(-c6ccc(-c7ccccc7)cc6)nc(-c6ccccc6)n5)c3)c3sc5ccccc5c34)cc21 ; CC1(C)c2ccccc2-c2ccc(-c3nc4ccccc4c4c3cc(-c3ccc5ccc6cccnc6c5n3)c3sc5ccccc5c34)cc21]

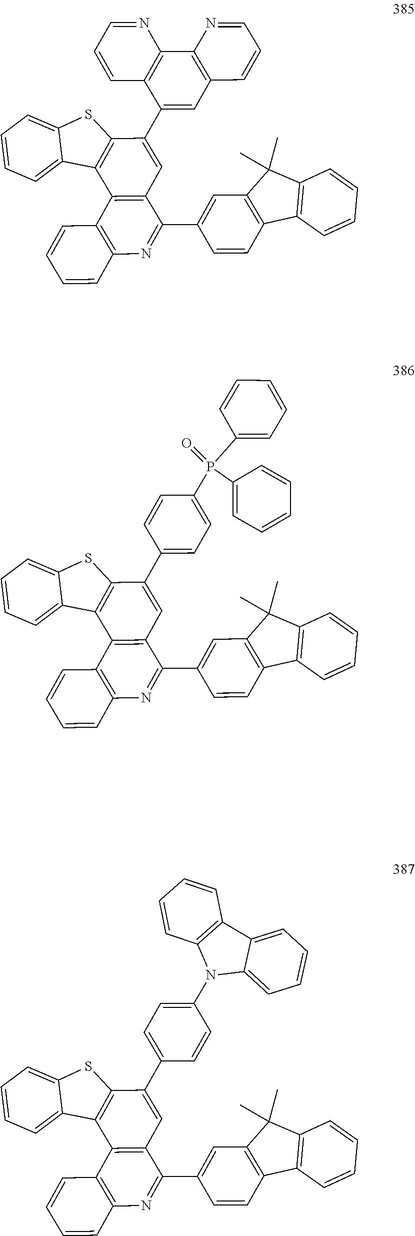 [SMILES: CC1(C)c2ccccc2-c2ccc(-c3nc4ccccc4c4c3cc(-c3cc5cccnc5c5ncccc35)c3sc5ccccc5c34)cc21 ; CC1(C)c2ccccc2-c2ccc(-c3nc4ccccc4c4c3cc(-c3ccc(P(=O)(c5ccccc5)c5ccccc5)cc3)c3sc5ccccc5c34)cc21 ; CC1(C)c2ccccc2-c2ccc(-c3nc4ccccc4c4c3cc(-c3ccc(-n5c6ccccc6c6ccccc65)cc3)c3sc5ccccc5c34)cc21]

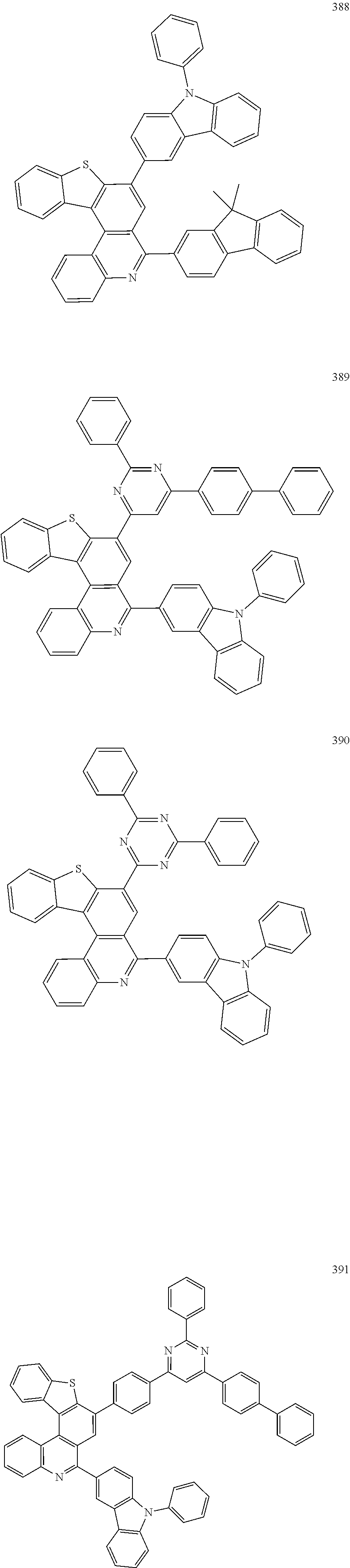 [SMILES: CC1(C)c2ccccc2-c2ccc(-c3nc4ccccc4c4c3cc(-c3ccc5c(c3)c3ccccc3n5-c3ccccc3)c3sc5ccccc5c34)cc21 ; c1ccc(-c2ccc(-c3cc(-c4cc5c(-c6ccc7c(c6)c6ccccc6n7-c6ccccc6)nc6ccccc6c5c5c4sc4ccccc45)nc(-c4ccccc4)n3)cc2)cc1 ; c1ccc(-c2nc(-c3ccccc3)nc(-c3cc4c(-c5ccc6c(c5)c5ccccc5n6-c5ccccc5)nc5ccccc5c4c4c3sc3ccccc34)n2)cc1 ; c1ccc(-c2ccc(-c3cc(-c4ccc(-c5cc6c(-c7ccc8c(c7)c7ccccc7n8-c7ccccc7)nc7ccccc7c6c6c5sc5ccccc56)cc4)nc(-c4ccccc4)n3)cc2)cc1]

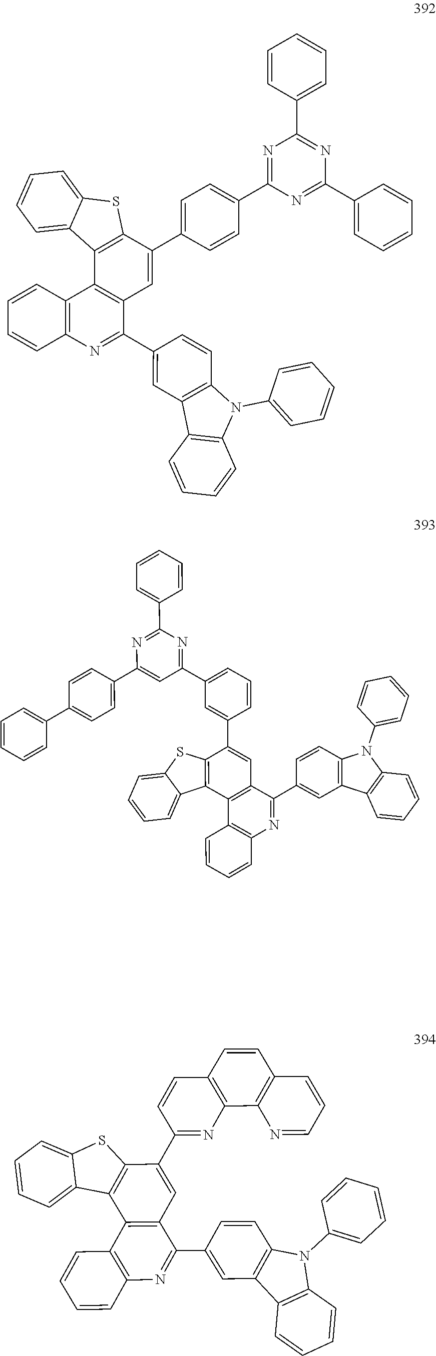 [SMILES: c1ccc(-c2nc(-c3ccccc3)nc(-c3ccc(-c4cc5c(-c6ccc7c(c6)c6ccccc6n7-c6ccccc6)nc6ccccc6c5c5c4sc4ccccc45)cc3)n2)cc1 ; c1ccc(-c2ccc(-c3cc(-c4cccc(-c5cc6c(-c7ccc8c(c7)c7ccccc7n8-c7ccccc7)nc7ccccc7c6c6c5sc5ccccc56)c4)nc(-c4ccccc4)n3)cc2)cc1 ; c1ccc(-n2c3ccccc3c3cc(-c4nc5ccccc5c5c4cc(-c4ccc6ccc7cccnc7c6n4)c4sc6ccccc6c45)ccc32)cc1]

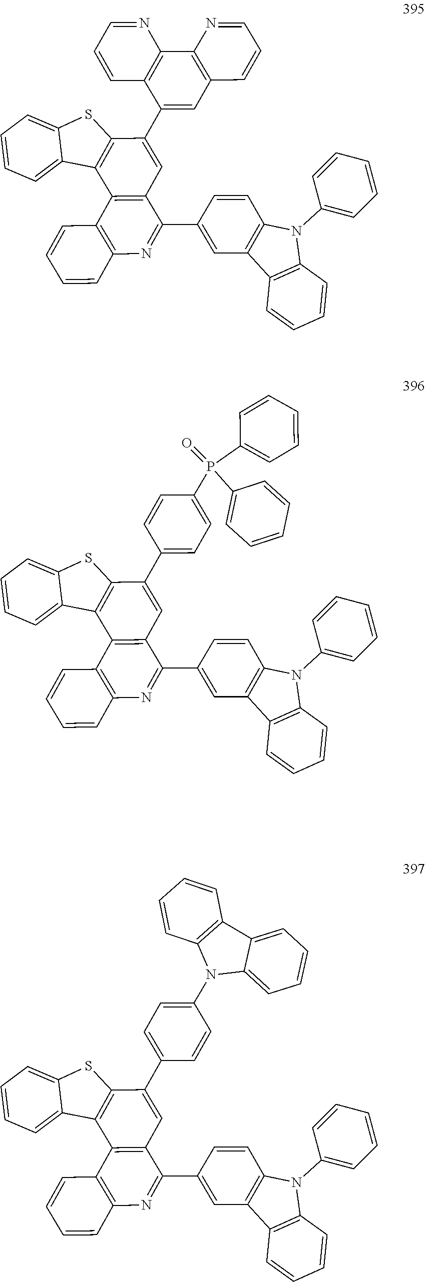 [SMILES: c1ccc(-n2c3ccccc3c3cc(-c4nc5ccccc5c5c4cc(-c4cc6cccnc6c6ncccc46)c4sc6ccccc6c45)ccc32)cc1 ; O=P(c1ccccc1)(c1ccccc1)c1ccc(-c2cc3c(-c4ccc5c(c4)c4ccccc4n5-c4ccccc4)nc4ccccc4c3c3c2sc2ccccc23)cc1 ; c1ccc(-n2c3ccccc3c3cc(-c4nc5ccccc5c5c4cc(-c4ccc(-n6c7ccccc7c7ccccc76)cc4)c4sc6ccccc6c45)ccc32)cc1]

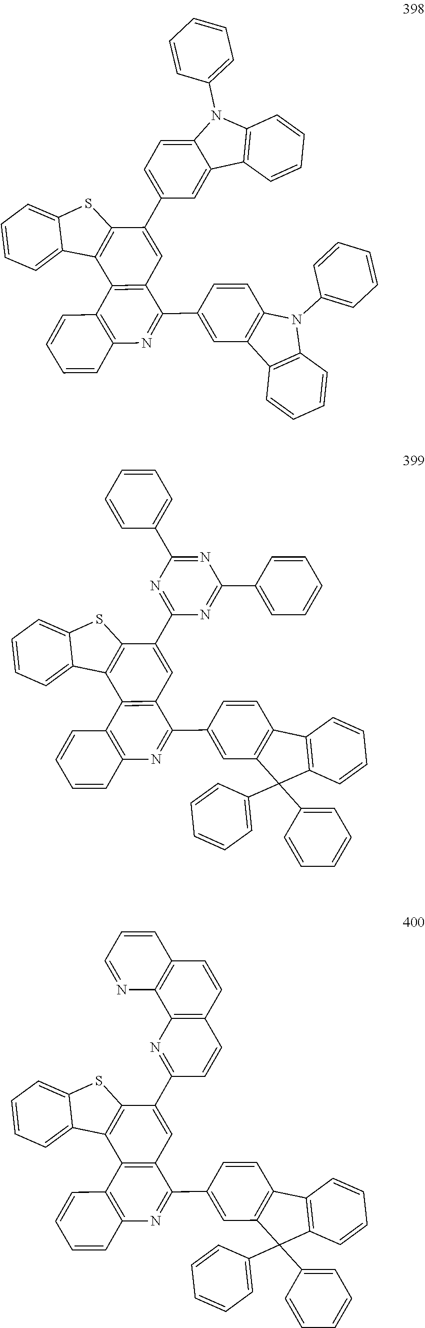 [SMILES: c1ccc(-n2c3ccccc3c3cc(-c4nc5ccccc5c5c4cc(-c4ccc6c(c4)c4ccccc4n6-c4ccccc4)c4sc6ccccc6c45)ccc32)cc1 ; c1ccc(-c2nc(-c3ccccc3)nc(-c3cc4c(-c5ccc6c(c5)C(c5ccccc5)(c5ccccc5)c5ccccc5-6)nc5ccccc5c4c4c3sc3ccccc34)n2)cc1 ; c1ccc(C2(c3ccccc3)c3ccccc3-c3ccc(-c4nc5ccccc5c5c4cc(-c4ccc6ccc7cccnc7c6n4)c4sc6ccccc6c45)cc32)cc1]

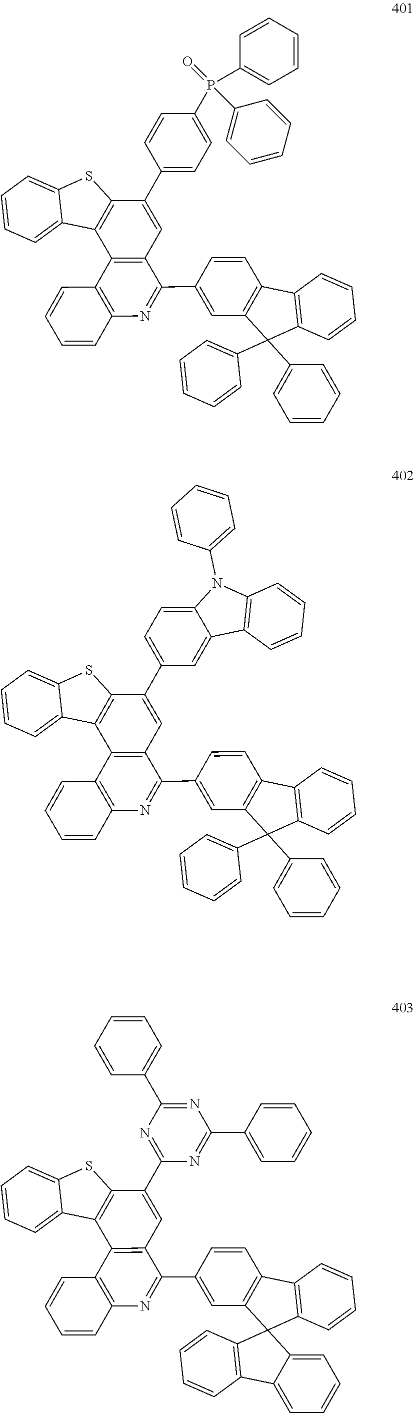 [SMILES: O=P(c1ccccc1)(c1ccccc1)c1ccc(-c2cc3c(-c4ccc5c(c4)C(c4ccccc4)(c4ccccc4)c4ccccc4-5)nc4ccccc4c3c3c2sc2ccccc23)cc1 ; c1ccc(-n2c3ccccc3c3cc(-c4cc5c(-c6ccc7c(c6)C(c6ccccc6)(c6ccccc6)c6ccccc6-7)nc6ccccc6c5c5c4sc4ccccc45)ccc32)cc1 ; c1ccc(-c2nc(-c3ccccc3)nc(-c3cc4c(-c5ccc6c(c5)C5(c7ccccc7-c7ccccc75)c5ccccc5-6)nc5ccccc5c4c4c3sc3ccccc34)n2)cc1]

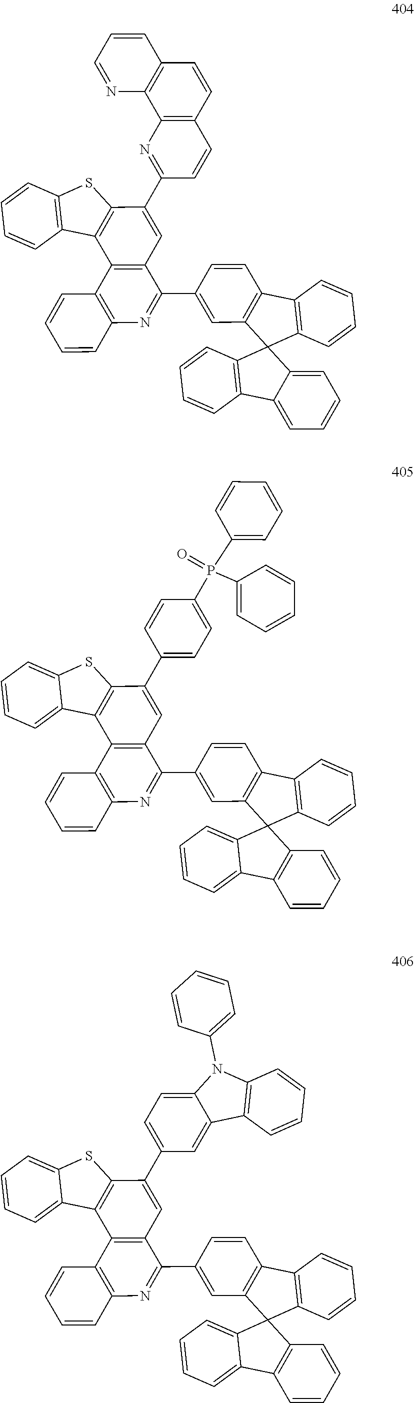 [SMILES: c1ccc2c(c1)-c1ccccc1C21c2ccccc2-c2ccc(-c3nc4ccccc4c4c3cc(-c3ccc5ccc6cccnc6c5n3)c3sc5ccccc5c34)cc21 ; O=P(c1ccccc1)(c1ccccc1)c1ccc(-c2cc3c(-c4ccc5c(c4)C4(c6ccccc6-c6ccccc64)c4ccccc4-5)nc4ccccc4c3c3c2sc2ccccc23)cc1 ; c1ccc(-n2c3ccccc3c3cc(-c4cc5c(-c6ccc7c(c6)C6(c8ccccc8-c8ccccc86)c6ccccc6-7)nc6ccccc6c5c5c4sc4ccccc45)ccc32)cc1]

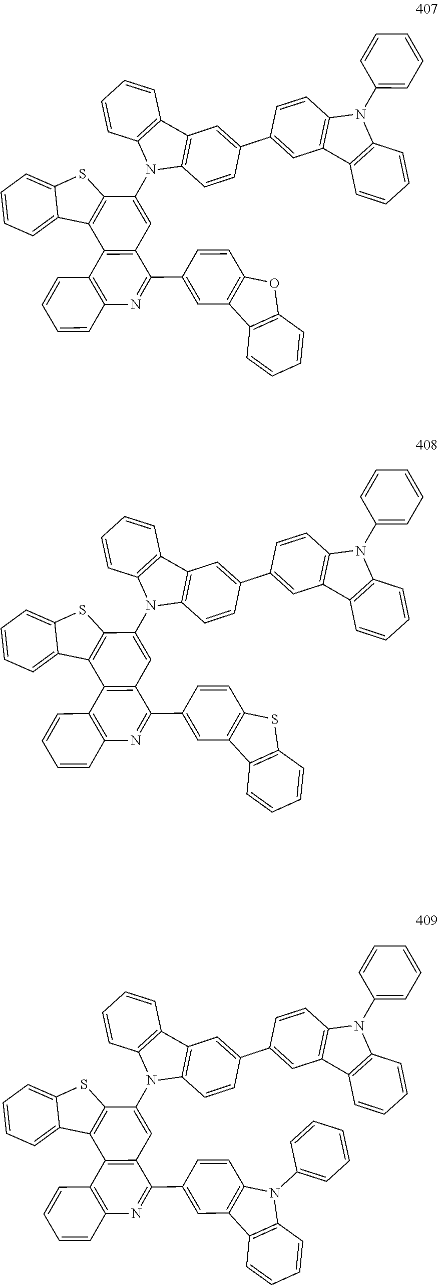 [SMILES: c1ccc(-n2c3ccccc3c3cc(-c4ccc5c(c4)c4ccccc4n5-c4cc5c(-c6ccc7oc8ccccc8c7c6)nc6ccccc6c5c5c4sc4ccccc45)ccc32)cc1 ; c1ccc(-n2c3ccccc3c3cc(-c4ccc5c(c4)c4ccccc4n5-c4cc5c(-c6ccc7sc8ccccc8c7c6)nc6ccccc6c5c5c4sc4ccccc45)ccc32)cc1 ; c1ccc(-n2c3ccccc3c3cc(-c4ccc5c(c4)c4ccccc4n5-c4cc5c(-c6ccc7c(c6)c6ccccc6n7-c6ccccc6)nc6ccccc6c5c5c4sc4ccccc45)ccc32)cc1]

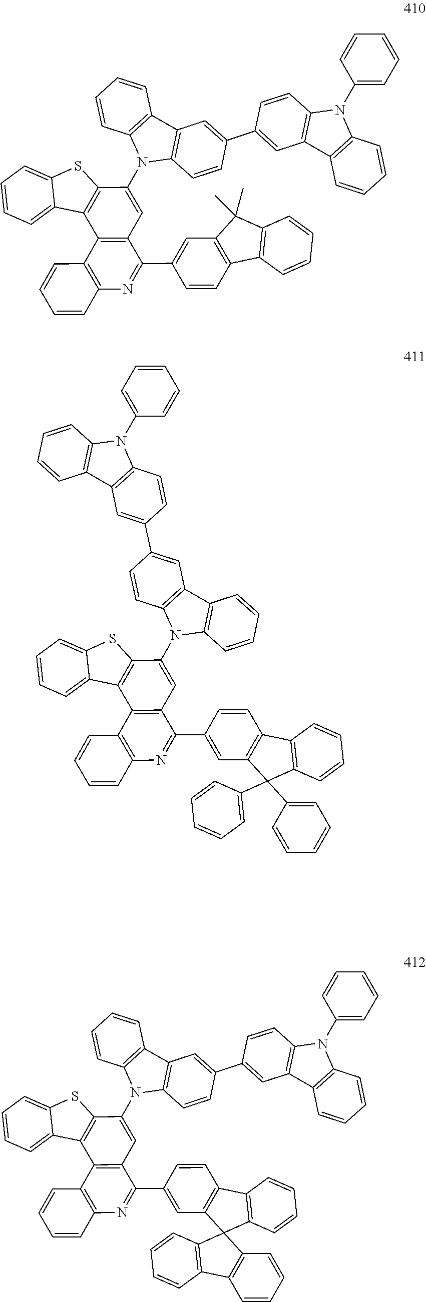 [SMILES: CC1(C)c2ccccc2-c2ccc(-c3nc4ccccc4c4c3cc(-n3c5ccccc5c5cc(-c6ccc7c(c6)c6ccccc6n7-c6ccccc6)ccc53)c3sc5ccccc5c34)cc21 ; c1ccc(-n2c3ccccc3c3cc(-c4ccc5c(c4)c4ccccc4n5-c4cc5c(-c6ccc7c(c6)C(c6ccccc6)(c6ccccc6)c6ccccc6-7)nc6ccccc6c5c5c4sc4ccccc45)ccc32)cc1 ; c1ccc(-n2c3ccccc3c3cc(-c4ccc5c(c4)c4ccccc4n5-c4cc5c(-c6ccc7c(c6)C6(c8ccccc8-c8ccccc86)c6ccccc6-7)nc6ccccc6c5c5c4sc4ccccc45)ccc32)cc1]

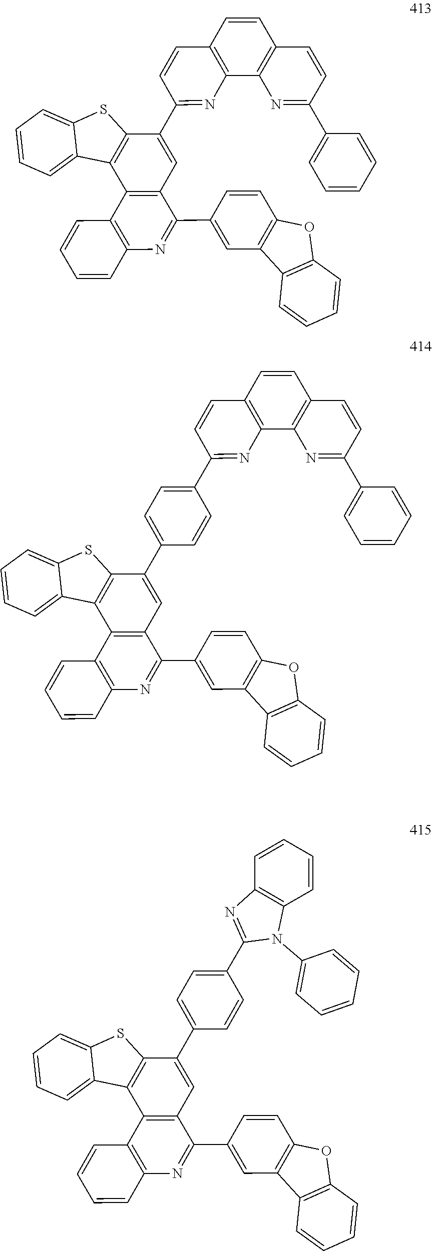 [SMILES: c1ccc(-c2ccc3ccc4ccc(-c5cc6c(-c7ccc8oc9ccccc9c8c7)nc7ccccc7c6c6c5sc5ccccc56)nc4c3n2)cc1 ; c1ccc(-c2ccc3ccc4ccc(-c5ccc(-c6cc7c(-c8ccc9oc%10ccccc%10c9c8)nc8ccccc8c7c7c6sc6ccccc67)cc5)nc4c3n2)cc1 ; c1ccc(-n2c(-c3ccc(-c4cc5c(-c6ccc7oc8ccccc8c7c6)nc6ccccc6c5c5c4sc4ccccc45)cc3)nc3ccccc32)cc1]

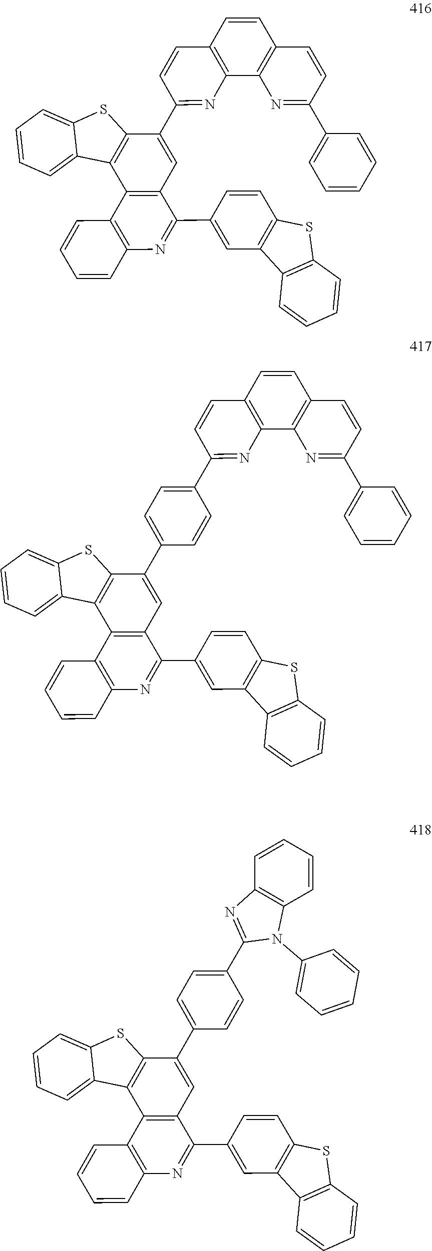 [SMILES: c1ccc(-c2ccc3ccc4ccc(-c5cc6c(-c7ccc8sc9ccccc9c8c7)nc7ccccc7c6c6c5sc5ccccc56)nc4c3n2)cc1 ; c1ccc(-c2ccc3ccc4ccc(-c5ccc(-c6cc7c(-c8ccc9sc%10ccccc%10c9c8)nc8ccccc8c7c7c6sc6ccccc67)cc5)nc4c3n2)cc1 ; c1ccc(-n2c(-c3ccc(-c4cc5c(-c6ccc7sc8ccccc8c7c6)nc6ccccc6c5c5c4sc4ccccc45)cc3)nc3ccccc32)cc1]

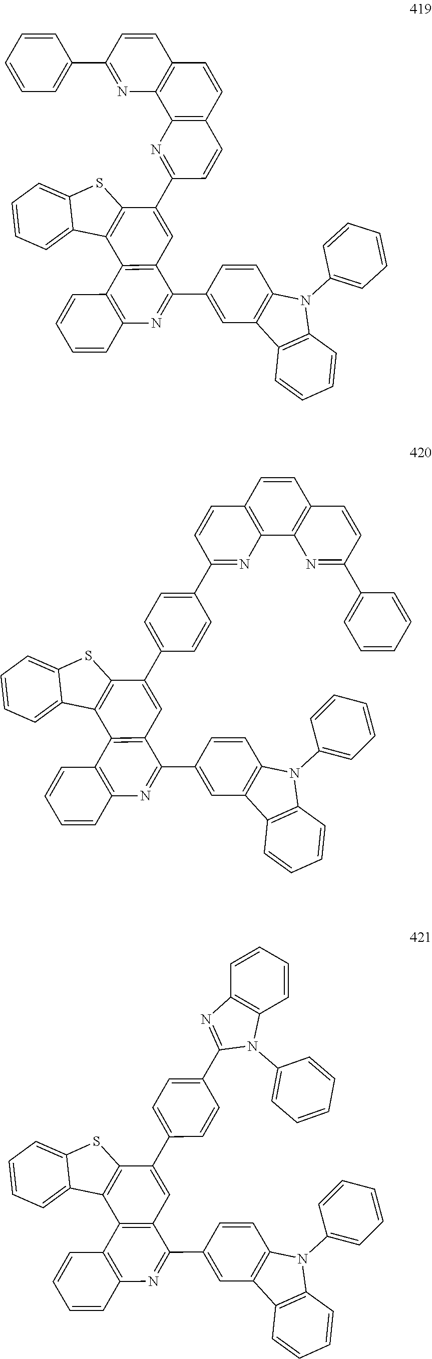 [SMILES: c1ccc(-c2ccc3ccc4ccc(-c5cc6c(-c7ccc8c(c7)c7ccccc7n8-c7ccccc7)nc7ccccc7c6c6c5sc5ccccc56)nc4c3n2)cc1 ; c1ccc(-c2ccc3ccc4ccc(-c5ccc(-c6cc7c(-c8ccc9c(c8)c8ccccc8n9-c8ccccc8)nc8ccccc8c7c7c6sc6ccccc67)cc5)nc4c3n2)cc1 ; c1ccc(-n2c(-c3ccc(-c4cc5c(-c6ccc7c(c6)c6ccccc6n7-c6ccccc6)nc6ccccc6c5c5c4sc4ccccc45)cc3)nc3ccccc32)cc1]

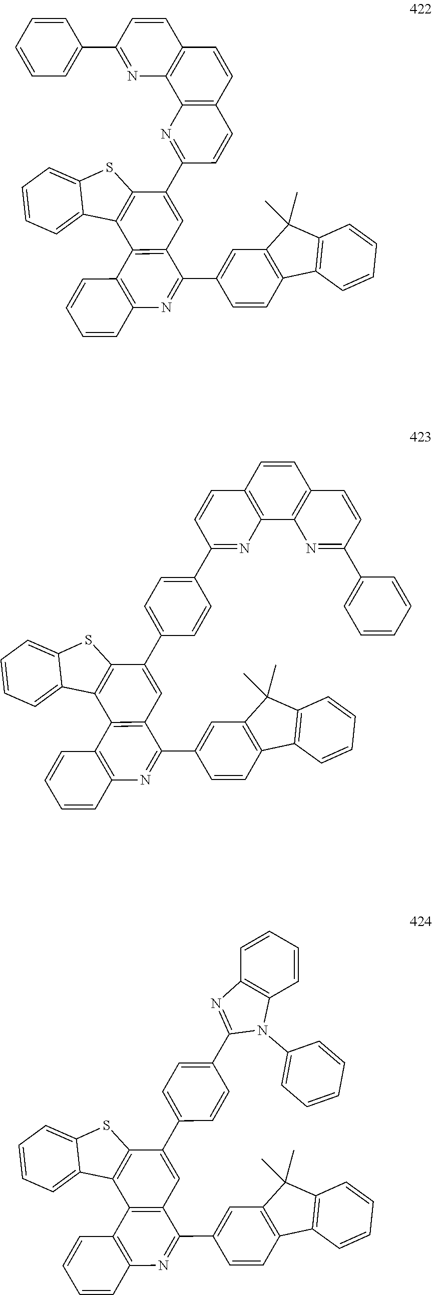 [SMILES: CC1(C)c2ccccc2-c2ccc(-c3nc4ccccc4c4c3cc(-c3ccc5ccc6ccc(-c7ccccc7)nc6c5n3)c3sc5ccccc5c34)cc21 ; CC1(C)c2ccccc2-c2ccc(-c3nc4ccccc4c4c3cc(-c3ccc(-c5ccc6ccc7ccc(-c8ccccc8)nc7c6n5)cc3)c3sc5ccccc5c34)cc21 ; CC1(C)c2ccccc2-c2ccc(-c3nc4ccccc4c4c3cc(-c3ccc(-c5nc6ccccc6n5-c5ccccc5)cc3)c3sc5ccccc5c34)cc21]

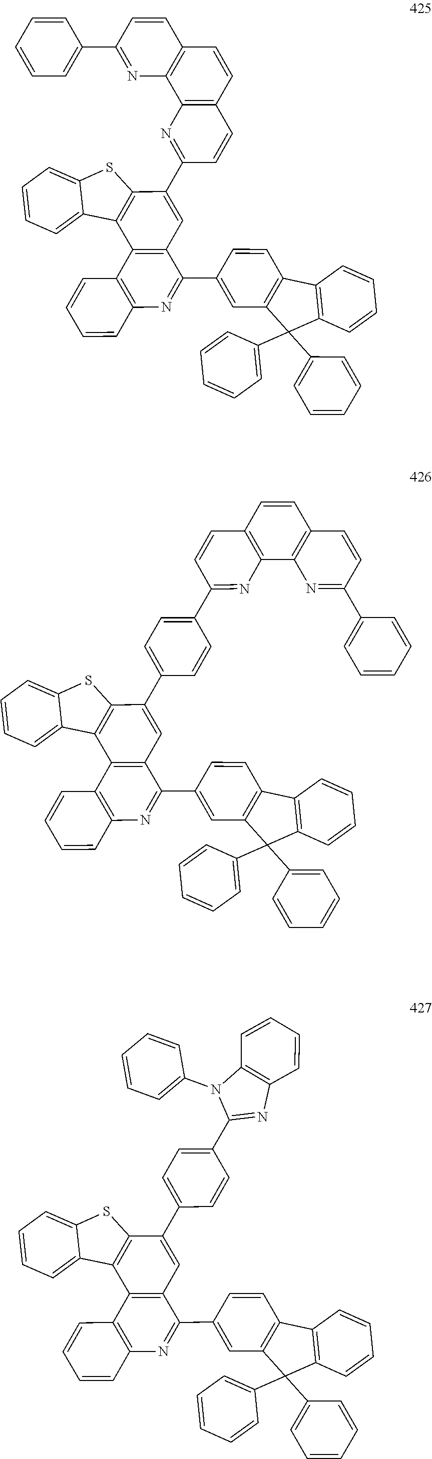 [SMILES: c1ccc(-c2ccc3ccc4ccc(-c5cc6c(-c7ccc8c(c7)C(c7ccccc7)(c7ccccc7)c7ccccc7-8)nc7ccccc7c6c6c5sc5ccccc56)nc4c3n2)cc1 ; c1ccc(-c2ccc3ccc4ccc(-c5ccc(-c6cc7c(-c8ccc9c(c8)C(c8ccccc8)(c8ccccc8)c8ccccc8-9)nc8ccccc8c7c7c6sc6ccccc67)cc5)nc4c3n2)cc1 ; c1ccc(-n2c(-c3ccc(-c4cc5c(-c6ccc7c(c6)C(c6ccccc6)(c6ccccc6)c6ccccc6-7)nc6ccccc6c5c5c4sc4ccccc45)cc3)nc3ccccc32)cc1]

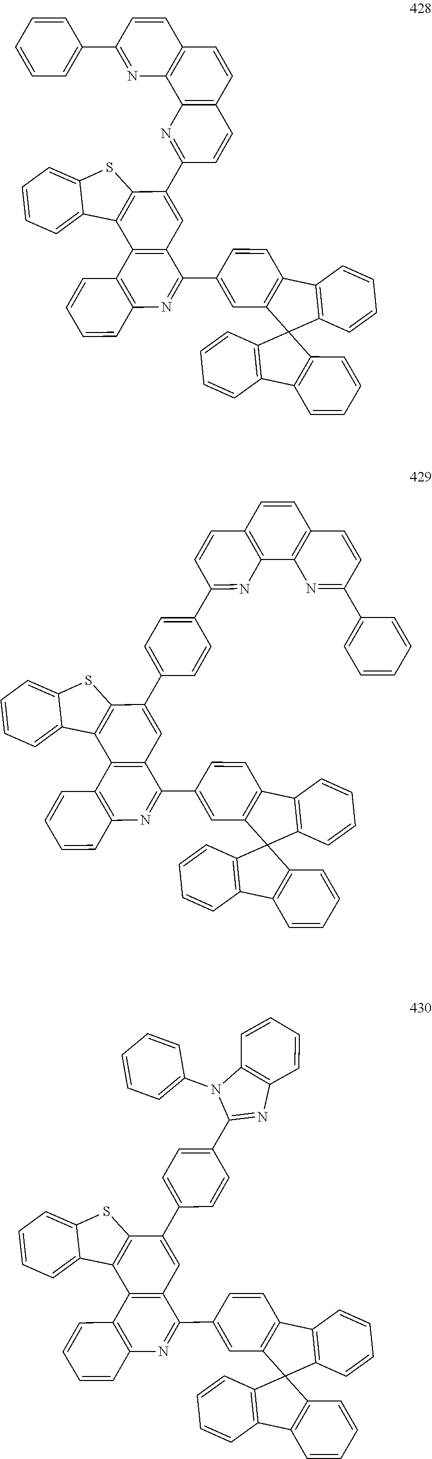 [SMILES: c1ccc(-c2ccc3ccc4ccc(-c5cc6c(-c7ccc8c(c7)C7(c9ccccc9-c9ccccc97)c7ccccc7-8)nc7ccccc7c6c6c5sc5ccccc56)nc4c3n2)cc1 ; c1ccc(-c2ccc3ccc4ccc(-c5ccc(-c6cc7c(-c8ccc9c(c8)C8(c%10ccccc%10-c%10ccccc%108)c8ccccc8-9)nc8ccccc8c7c7c6sc6ccccc67)cc5)nc4c3n2)cc1 ; c1ccc(-n2c(-c3ccc(-c4cc5c(-c6ccc7c(c6)C6(c8ccccc8-c8ccccc86)c6ccccc6-7)nc6ccccc6c5c5c4sc4ccccc45)cc3)nc3ccccc32)cc1]

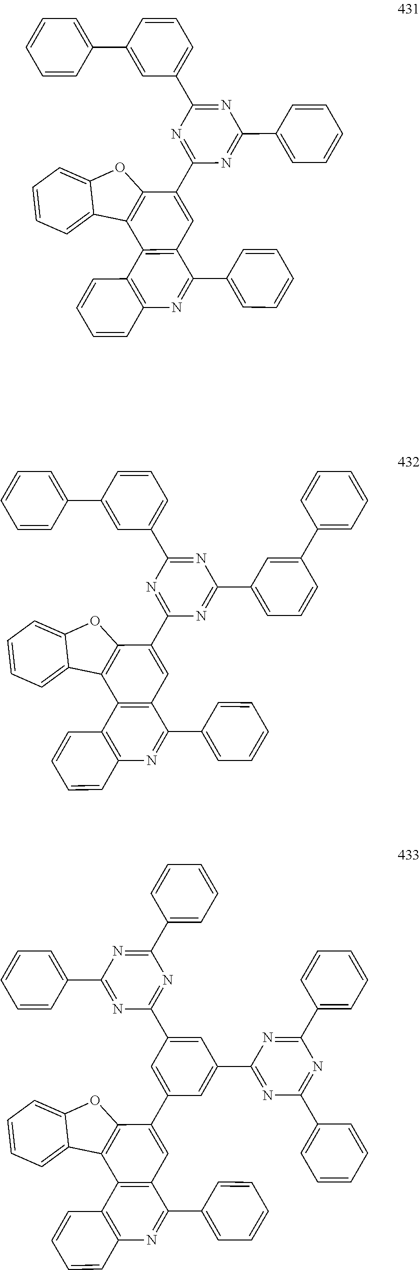 [SMILES: c1ccc(-c2cccc(-c3nc(-c4ccccc4)nc(-c4cc5c(-c6ccccc6)nc6ccccc6c5c5c4oc4ccccc45)n3)c2)cc1 ; c1ccc(-c2cccc(-c3nc(-c4cccc(-c5ccccc5)c4)nc(-c4cc5c(-c6ccccc6)nc6ccccc6c5c5c4oc4ccccc45)n3)c2)cc1 ; c1ccc(-c2nc(-c3ccccc3)nc(-c3cc(-c4nc(-c5ccccc5)nc(-c5ccccc5)n4)cc(-c4cc5c(-c6ccccc6)nc6ccccc6c5c5c4oc4ccccc45)c3)n2)cc1]

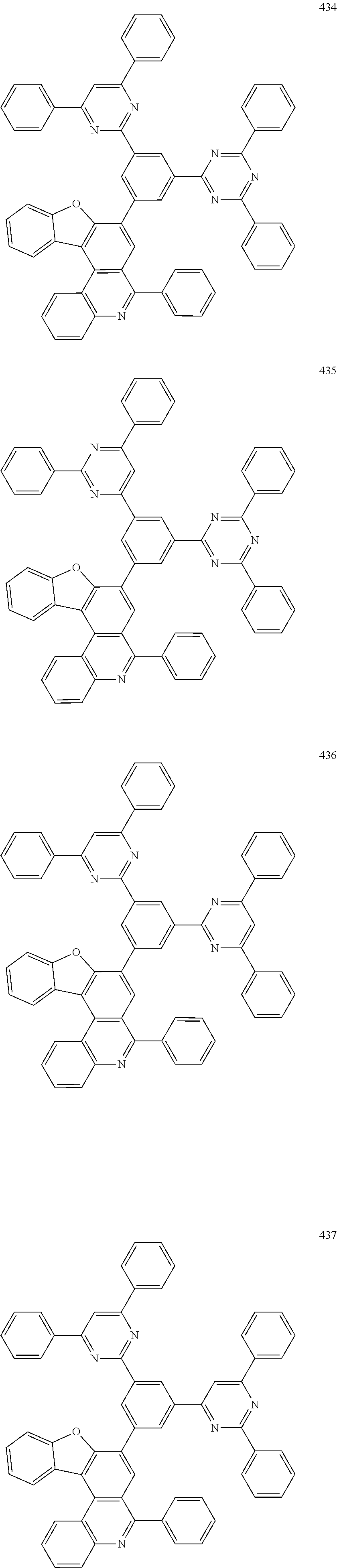 [SMILES: c1ccc(-c2cc(-c3ccccc3)nc(-c3cc(-c4nc(-c5ccccc5)nc(-c5ccccc5)n4)cc(-c4cc5c(-c6ccccc6)nc6ccccc6c5c5c4oc4ccccc45)c3)n2)cc1 ; c1ccc(-c2cc(-c3cc(-c4nc(-c5ccccc5)nc(-c5ccccc5)n4)cc(-c4cc5c(-c6ccccc6)nc6ccccc6c5c5c4oc4ccccc45)c3)nc(-c3ccccc3)n2)cc1 ; c1ccc(-c2cc(-c3ccccc3)nc(-c3cc(-c4nc(-c5ccccc5)cc(-c5ccccc5)n4)cc(-c4cc5c(-c6ccccc6)nc6ccccc6c5c5c4oc4ccccc45)c3)n2)cc1 ; c1ccc(-c2cc(-c3cc(-c4nc(-c5ccccc5)cc(-c5ccccc5)n4)cc(-c4cc5c(-c6ccccc6)nc6ccccc6c5c5c4oc4ccccc45)c3)nc(-c3ccccc3)n2)cc1]

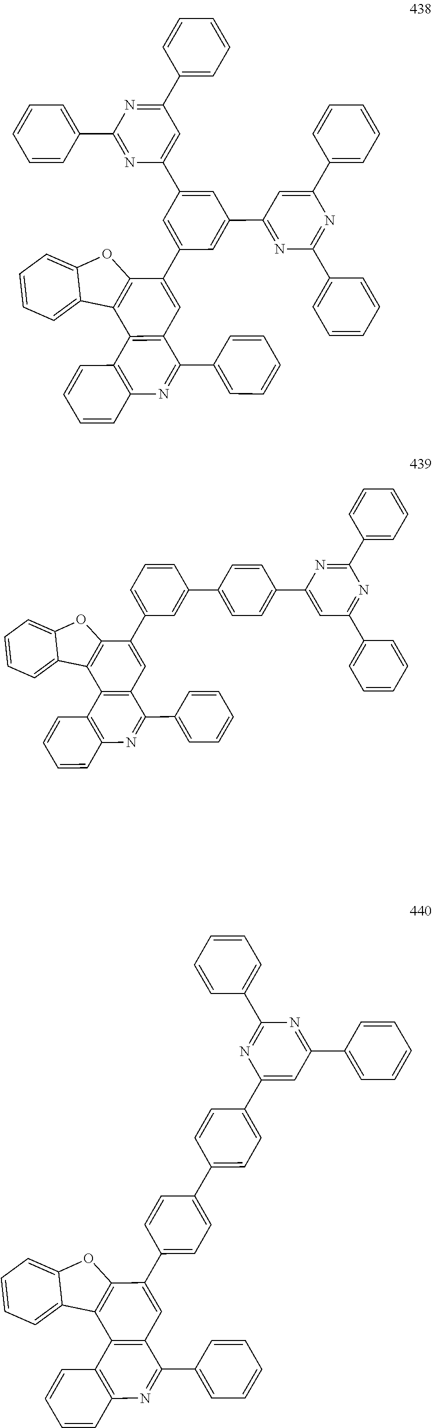 [SMILES: c1ccc(-c2cc(-c3cc(-c4cc(-c5ccccc5)nc(-c5ccccc5)n4)cc(-c4cc5c(-c6ccccc6)nc6ccccc6c5c5c4oc4ccccc45)c3)nc(-c3ccccc3)n2)cc1 ; c1ccc(-c2cc(-c3ccc(-c4cccc(-c5cc6c(-c7ccccc7)nc7ccccc7c6c6c5oc5ccccc56)c4)cc3)nc(-c3ccccc3)n2)cc1 ; c1ccc(-c2cc(-c3ccc(-c4ccc(-c5cc6c(-c7ccccc7)nc7ccccc7c6c6c5oc5ccccc56)cc4)cc3)nc(-c3ccccc3)n2)cc1]

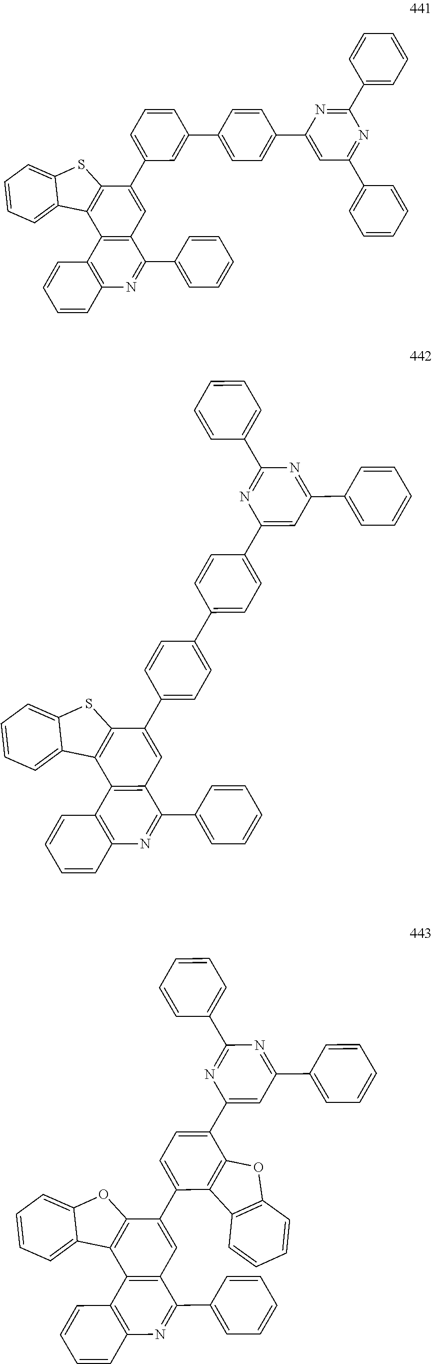 [SMILES: c1ccc(-c2cc(-c3ccc(-c4cccc(-c5cc6c(-c7ccccc7)nc7ccccc7c6c6c5sc5ccccc56)c4)cc3)nc(-c3ccccc3)n2)cc1 ; c1ccc(-c2cc(-c3ccc(-c4ccc(-c5cc6c(-c7ccccc7)nc7ccccc7c6c6c5sc5ccccc56)cc4)cc3)nc(-c3ccccc3)n2)cc1 ; c1ccc(-c2cc(-c3ccc(-c4cc5c(-c6ccccc6)nc6ccccc6c5c5c4oc4ccccc45)c4c3oc3ccccc34)nc(-c3ccccc3)n2)cc1]

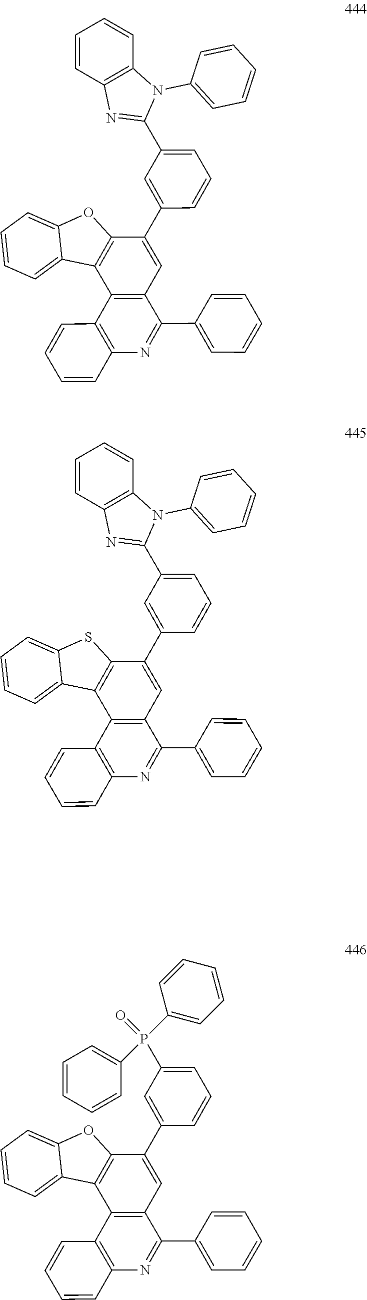 [SMILES: c1ccc(-c2nc3ccccc3c3c2cc(-c2cccc(-c4nc5ccccc5n4-c4ccccc4)c2)c2oc4ccccc4c23)cc1 ; c1ccc(-c2nc3ccccc3c3c2cc(-c2cccc(-c4nc5ccccc5n4-c4ccccc4)c2)c2sc4ccccc4c23)cc1 ; O=P(c1ccccc1)(c1ccccc1)c1cccc(-c2cc3c(-c4ccccc4)nc4ccccc4c3c3c2oc2ccccc23)c1]

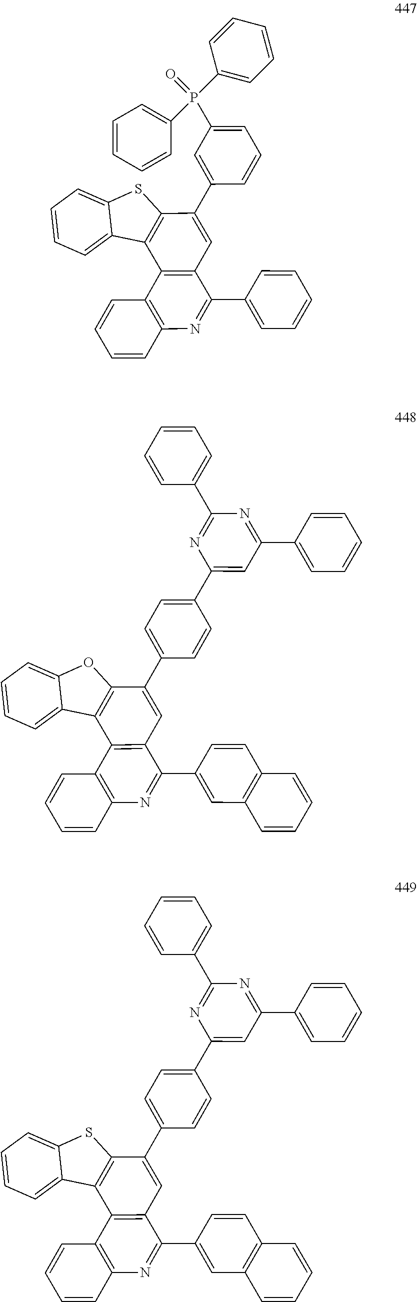 [SMILES: O=P(c1ccccc1)(c1ccccc1)c1cccc(-c2cc3c(-c4ccccc4)nc4ccccc4c3c3c2sc2ccccc23)c1 ; c1ccc(-c2cc(-c3ccc(-c4cc5c(-c6ccc7ccccc7c6)nc6ccccc6c5c5c4oc4ccccc45)cc3)nc(-c3ccccc3)n2)cc1 ; c1ccc(-c2cc(-c3ccc(-c4cc5c(-c6ccc7ccccc7c6)nc6ccccc6c5c5c4sc4ccccc45)cc3)nc(-c3ccccc3)n2)cc1]

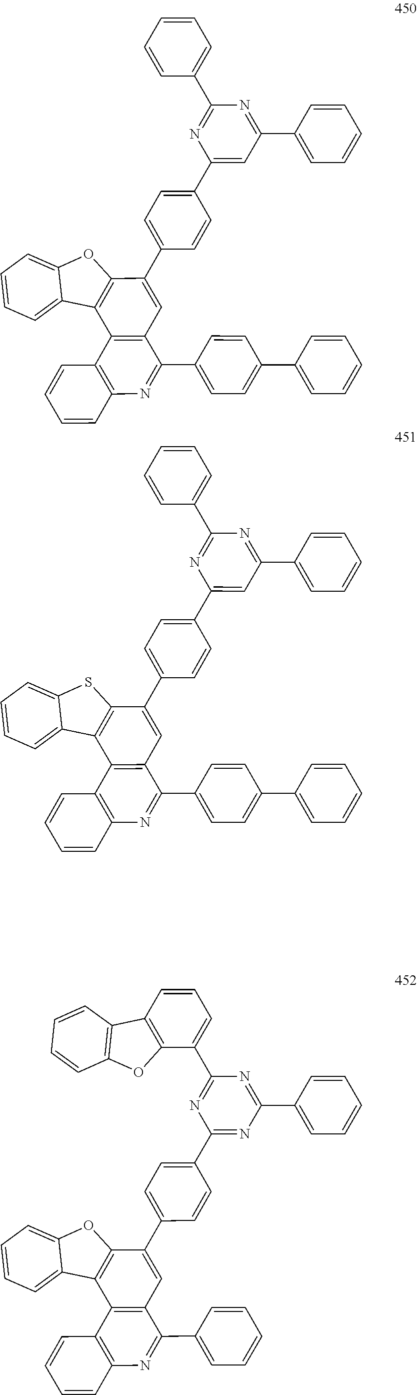 [SMILES: c1ccc(-c2ccc(-c3nc4ccccc4c4c3cc(-c3ccc(-c5cc(-c6ccccc6)nc(-c6ccccc6)n5)cc3)c3oc5ccccc5c34)cc2)cc1 ; c1ccc(-c2ccc(-c3nc4ccccc4c4c3cc(-c3ccc(-c5cc(-c6ccccc6)nc(-c6ccccc6)n5)cc3)c3sc5ccccc5c34)cc2)cc1 ; c1ccc(-c2nc(-c3ccc(-c4cc5c(-c6ccccc6)nc6ccccc6c5c5c4oc4ccccc45)cc3)nc(-c3cccc4c3oc3ccccc34)n2)cc1]

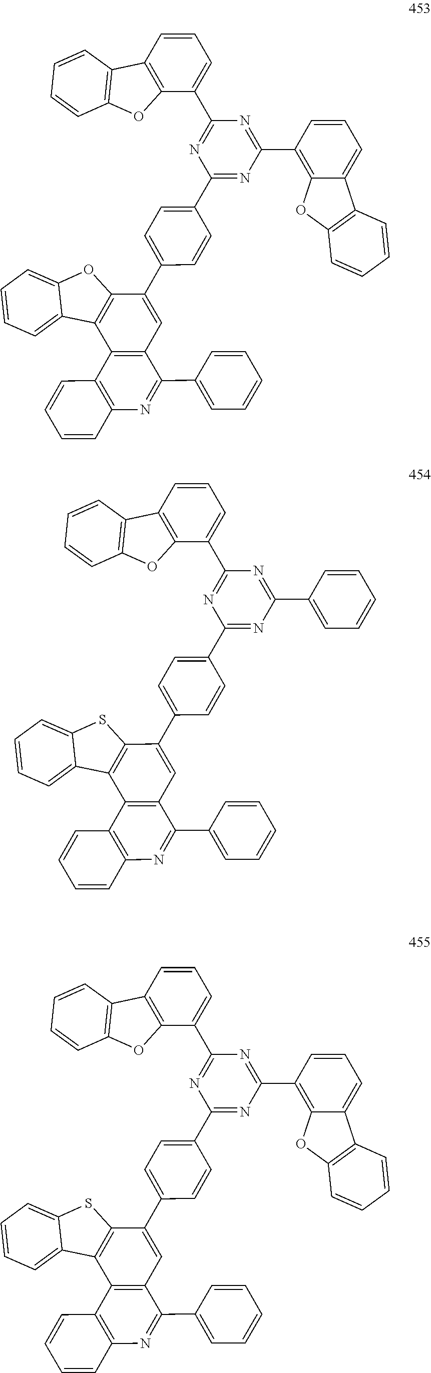 [SMILES: c1ccc(-c2nc3ccccc3c3c2cc(-c2ccc(-c4nc(-c5cccc6c5oc5ccccc56)nc(-c5cccc6c5oc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4c3oc3ccccc34)n2)cc1 ; c1ccc(-c2nc3ccccc3c3c2cc(-c2ccc(-c4nc(-c5cccc6c5oc5ccccc56)nc(-c5cccc6c5oc5ccccc56)n4)cc2)c2sc4ccccc4c23)cc1]

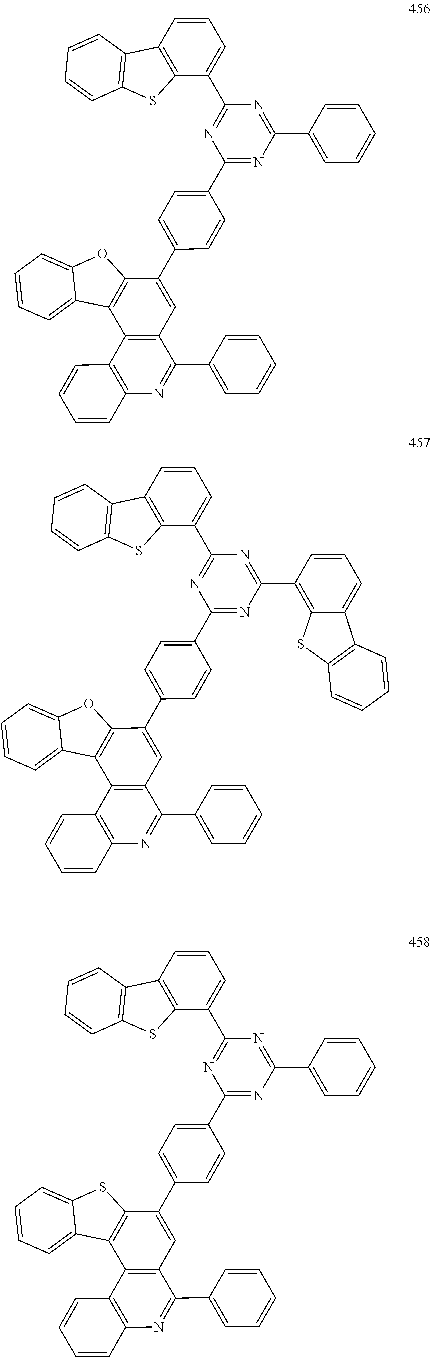 [SMILES: c1ccc(-c2nc(-c3ccc(-c4cc5c(-c6ccccc6)nc6ccccc6c5c5c4oc4ccccc45)cc3)nc(-c3cccc4c3sc3ccccc34)n2)cc1 ; c1ccc(-c2nc3ccccc3c3c2cc(-c2ccc(-c4nc(-c5cccc6c5sc5ccccc56)nc(-c5cccc6c5sc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4c3sc3ccccc34)n2)cc1]

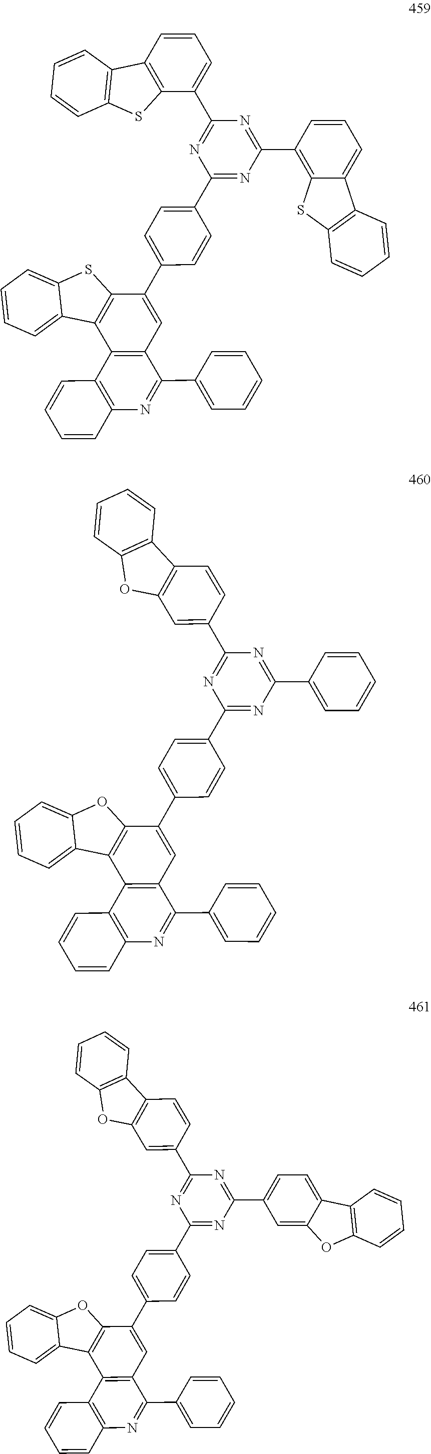 [SMILES: c1ccc(-c2nc3ccccc3c3c2cc(-c2ccc(-c4nc(-c5cccc6c5sc5ccccc56)nc(-c5cccc6c5sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4c(c3)oc3ccccc34)n2)cc1 ; c1ccc(-c2nc3ccccc3c3c2cc(-c2ccc(-c4nc(-c5ccc6c(c5)oc5ccccc56)nc(-c5ccc6c(c5)oc5ccccc56)n4)cc2)c2oc4ccccc4c23)cc1]

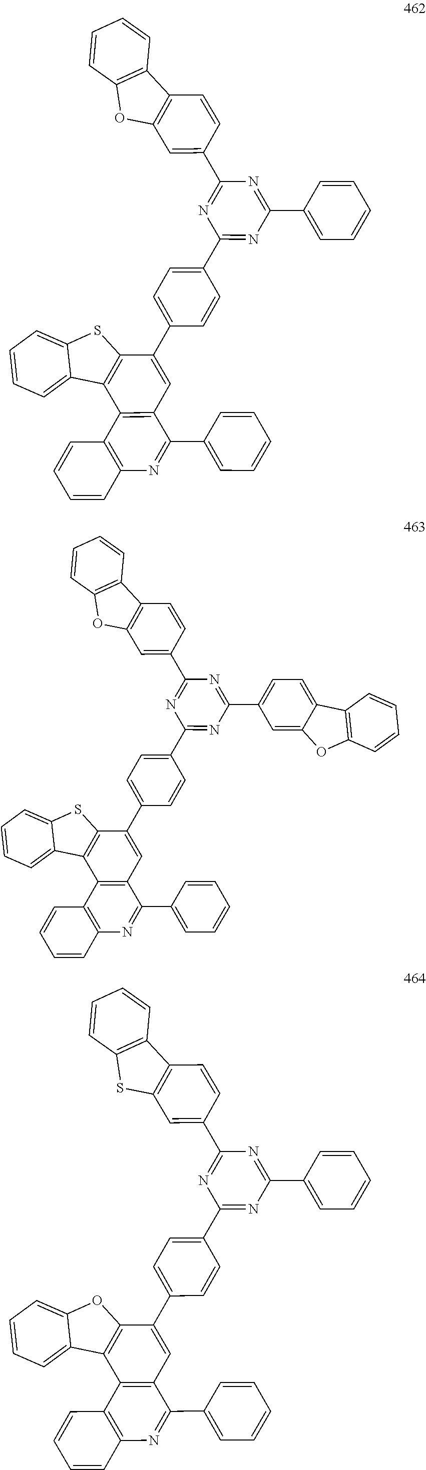

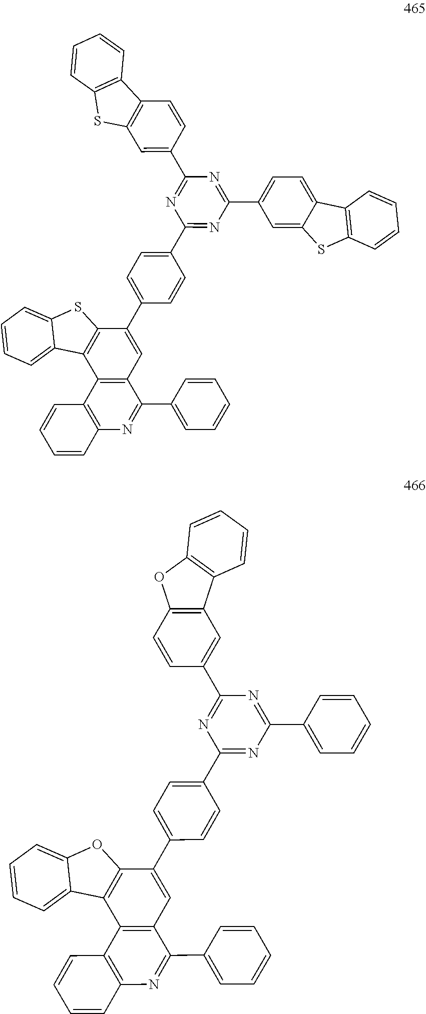 [SMILES: c1ccc(-c2nc3ccccc3c3c2cc(-c2ccc(-c4nc(-c5ccc6c(c5)sc5ccccc56)nc(-c5ccc6c(c5)sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4oc5ccccc5c4c3)n2)cc1]

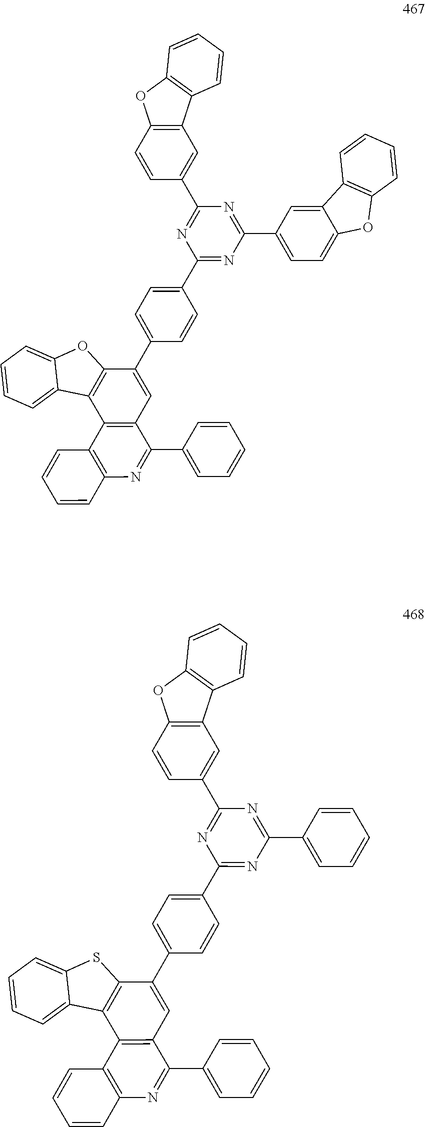 [SMILES: c1ccc(-c2nc3ccccc3c3c2cc(-c2ccc(-c4nc(-c5ccc6oc7ccccc7c6c5)nc(-c5ccc6oc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3ccc4oc5ccccc5c4c3)n2)cc1]

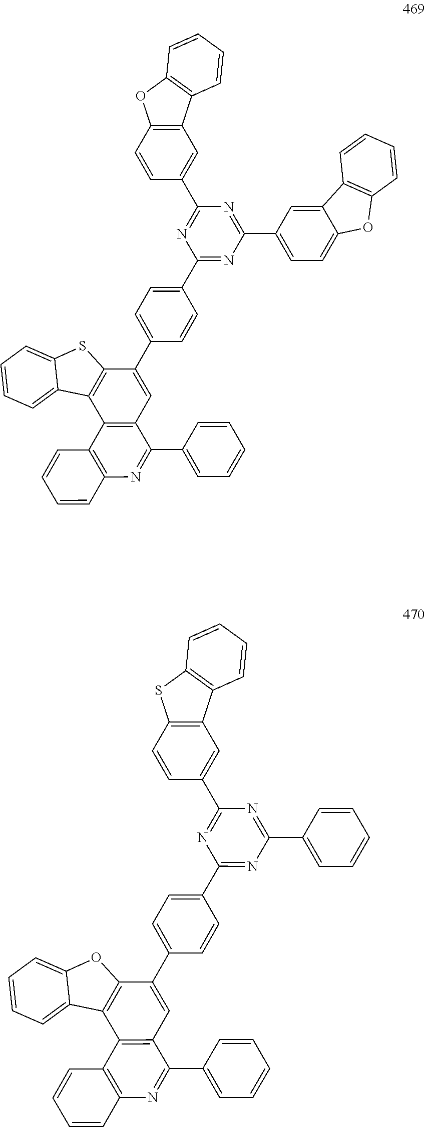 [SMILES: c1ccc(-c2nc3ccccc3c3c2cc(-c2ccc(-c4nc(-c5ccc6oc7ccccc7c6c5)nc(-c5ccc6oc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3ccc4sc5ccccc5c4c3)n2)cc1]

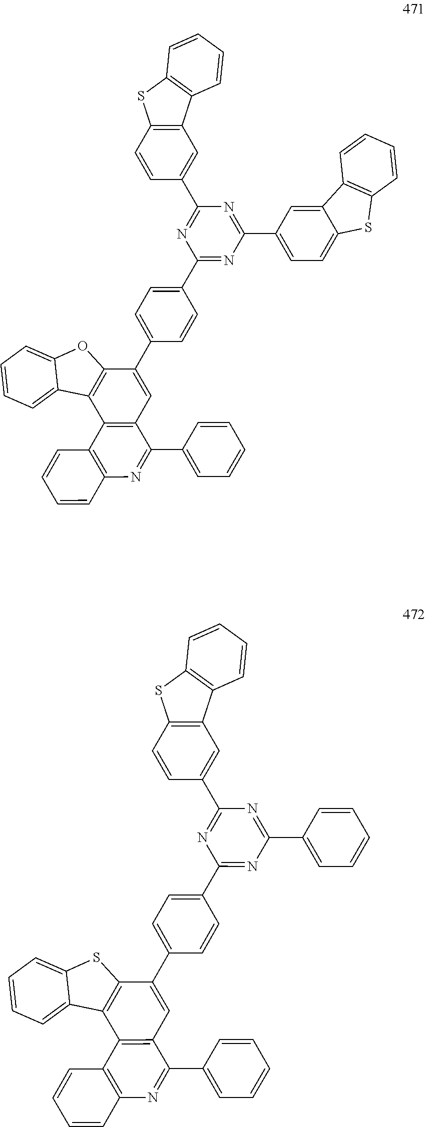 [SMILES: c1ccc(-c2nc3ccccc3c3c2cc(-c2ccc(-c4nc(-c5ccc6sc7ccccc7c6c5)nc(-c5ccc6sc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3ccc4sc5ccccc5c4c3)n2)cc1]

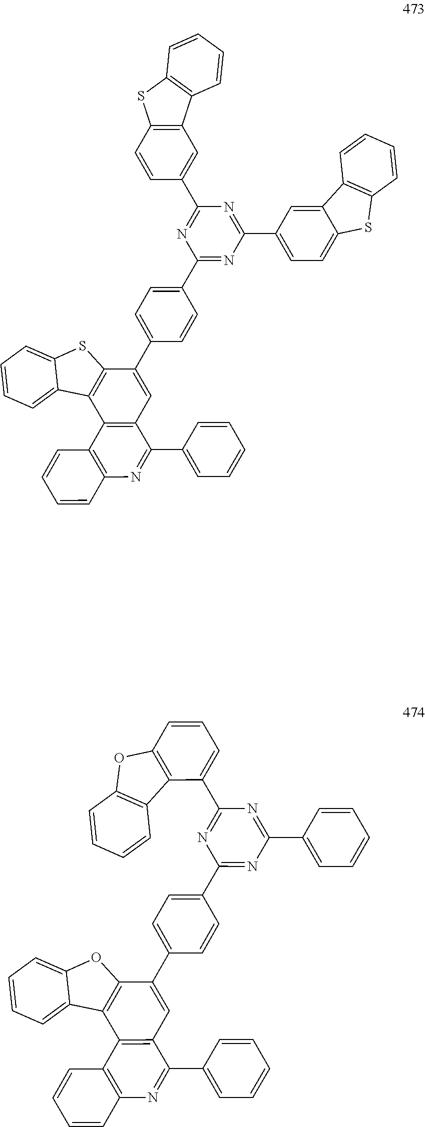 [SMILES: c1ccc(-c2nc3ccccc3c3c2cc(-c2ccc(-c4nc(-c5ccc6sc7ccccc7c6c5)nc(-c5ccc6sc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3cccc4oc5ccccc5c34)n2)cc1]

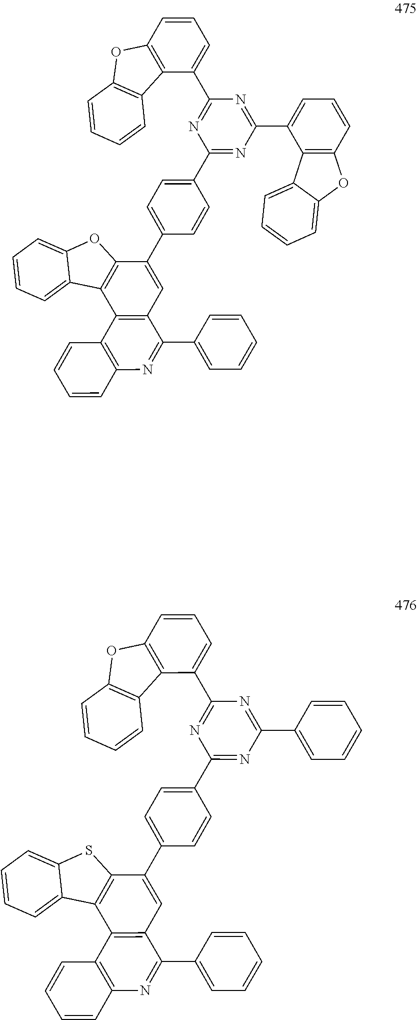 [SMILES: c1ccc(-c2nc3ccccc3c3c2cc(-c2ccc(-c4nc(-c5cccc6oc7ccccc7c56)nc(-c5cccc6oc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4oc5ccccc5c34)n2)cc1]

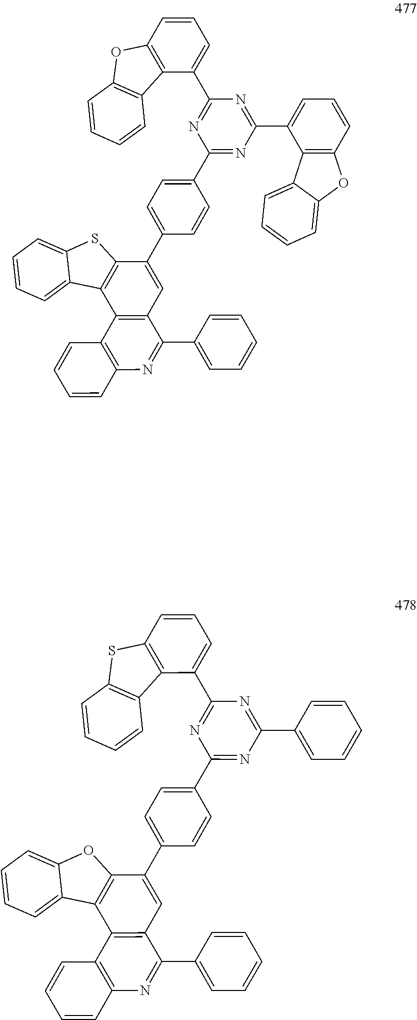 [SMILES: c1ccc(-c2nc3ccccc3c3c2cc(-c2ccc(-c4nc(-c5cccc6oc7ccccc7c56)nc(-c5cccc6oc7ccccc7c56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)nc(-c3cccc4sc5ccccc5c34)n2)cc1]

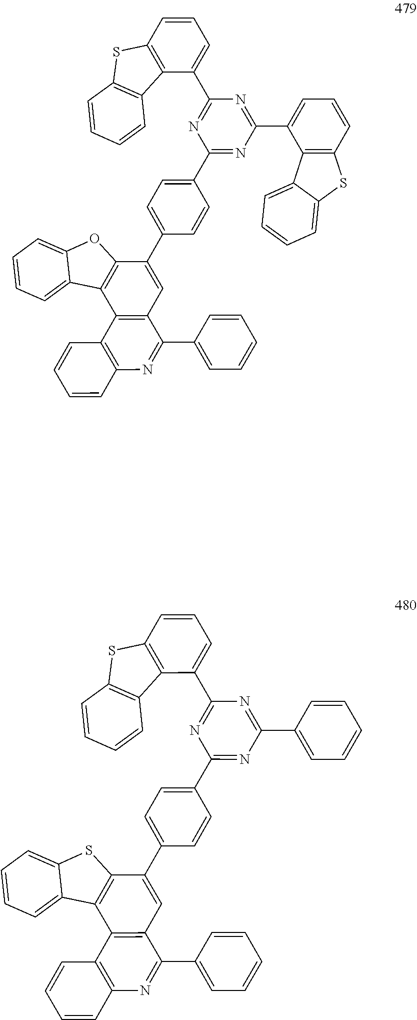 [SMILES: c1ccc(-c2nc3ccccc3c3c2cc(-c2ccc(-c4nc(-c5cccc6sc7ccccc7c56)nc(-c5cccc6sc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)nc(-c3cccc4sc5ccccc5c34)n2)cc1]

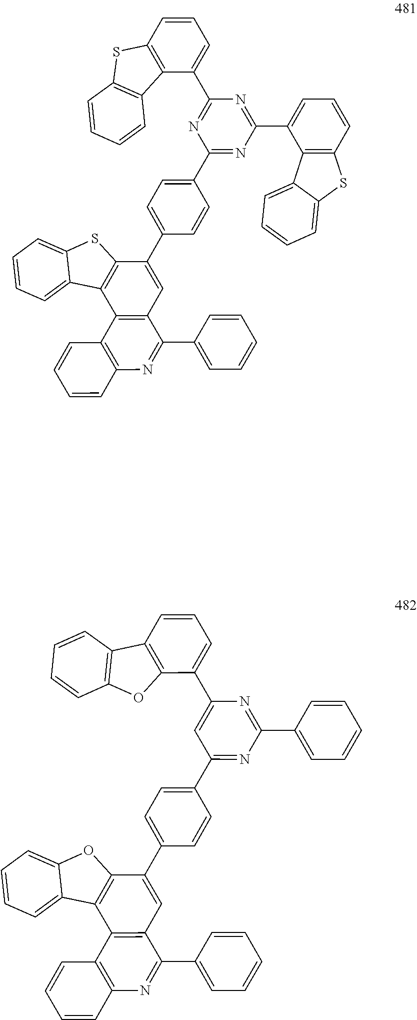 [SMILES: c1ccc(-c2nc3ccccc3c3c2cc(-c2ccc(-c4nc(-c5cccc6sc7ccccc7c56)nc(-c5cccc6sc7ccccc7c56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4c3oc3ccccc34)n2)cc1]

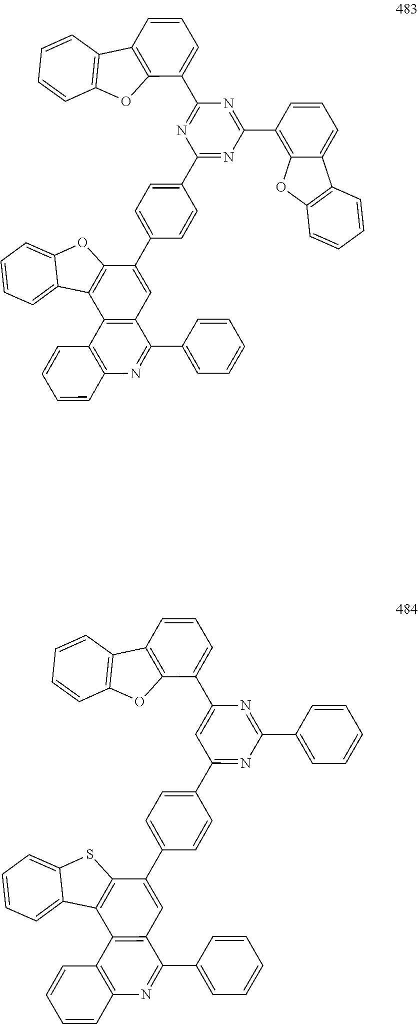 [SMILES: c1ccc(-c2nc3ccccc3c3c2cc(-c2ccc(-c4nc(-c5cccc6c5oc5ccccc56)nc(-c5cccc6c5oc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4c3oc3ccccc34)n2)cc1]

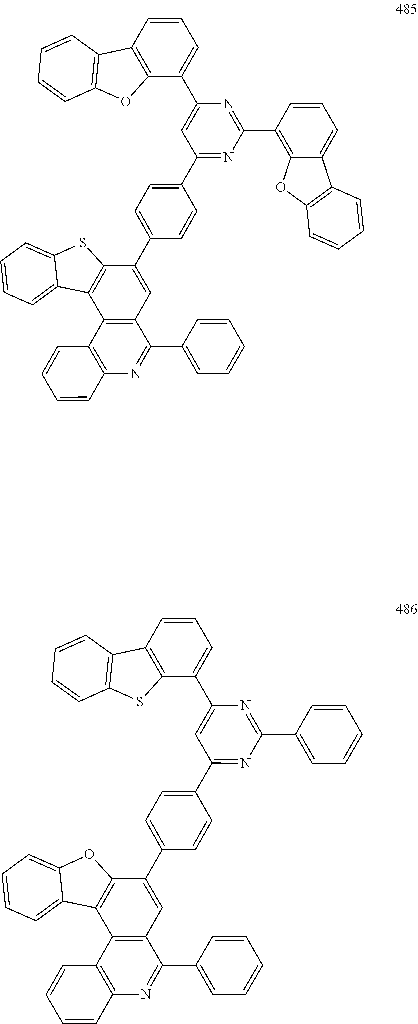 [SMILES: c1ccc(-c2nc3ccccc3c3c2cc(-c2ccc(-c4cc(-c5cccc6c5oc5ccccc56)nc(-c5cccc6c5oc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4c3sc3ccccc34)n2)cc1]

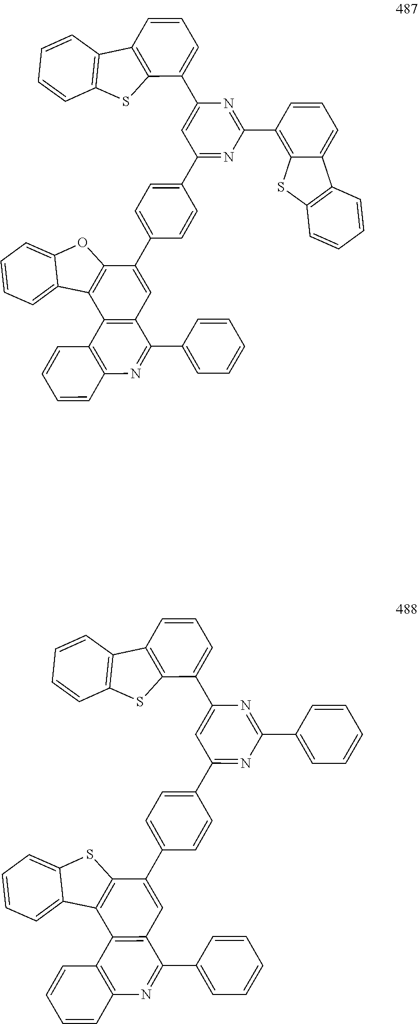 [SMILES: c1ccc(-c2nc3ccccc3c3c2cc(-c2ccc(-c4cc(-c5cccc6c5sc5ccccc56)nc(-c5cccc6c5sc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4c3sc3ccccc34)n2)cc1]

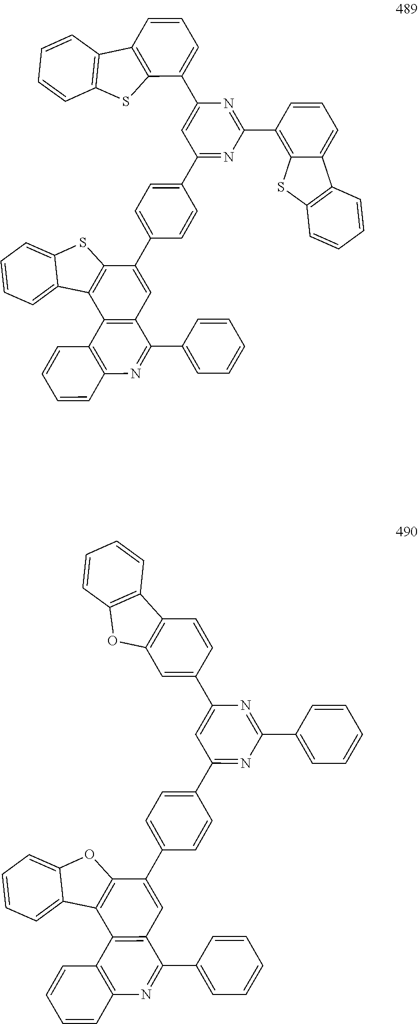 [SMILES: c1ccc(-c2nc3ccccc3c3c2cc(-c2ccc(-c4cc(-c5cccc6c5sc5ccccc56)nc(-c5cccc6c5sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3ccc4c(c3)oc3ccccc34)n2)cc1]

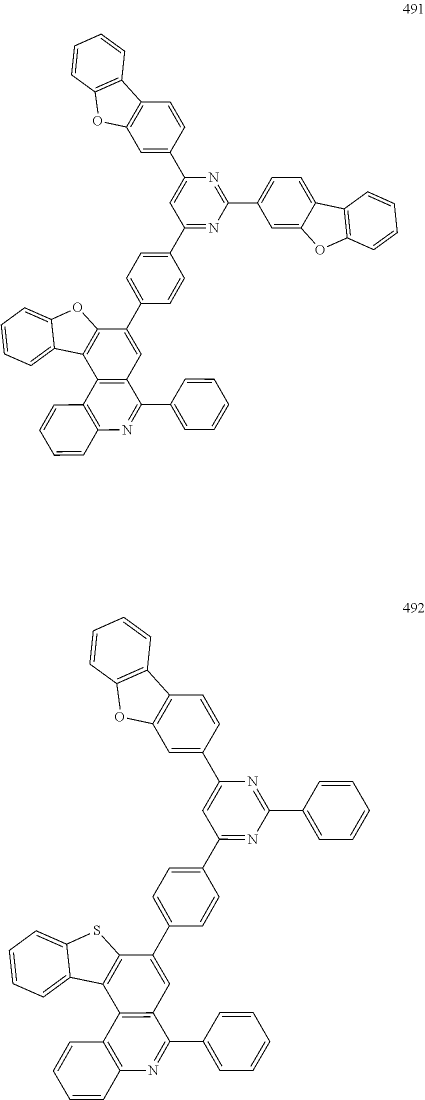 [SMILES: c1ccc(-c2nc3ccccc3c3c2cc(-c2ccc(-c4cc(-c5ccc6c(c5)oc5ccccc56)nc(-c5ccc6c(c5)oc5ccccc56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3ccc4c(c3)oc3ccccc34)n2)cc1]

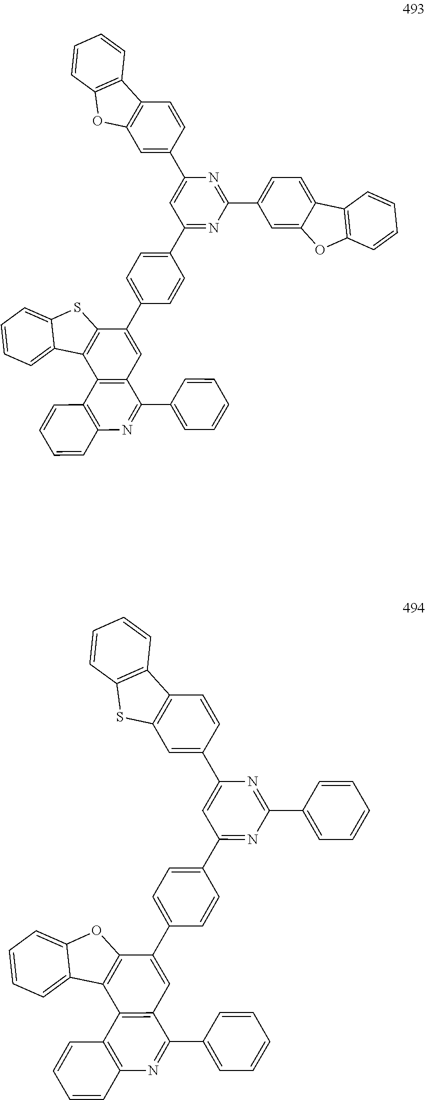 [SMILES: c1ccc(-c2nc3ccccc3c3c2cc(-c2ccc(-c4cc(-c5ccc6c(c5)oc5ccccc56)nc(-c5ccc6c(c5)oc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3ccc4c(c3)sc3ccccc34)n2)cc1]

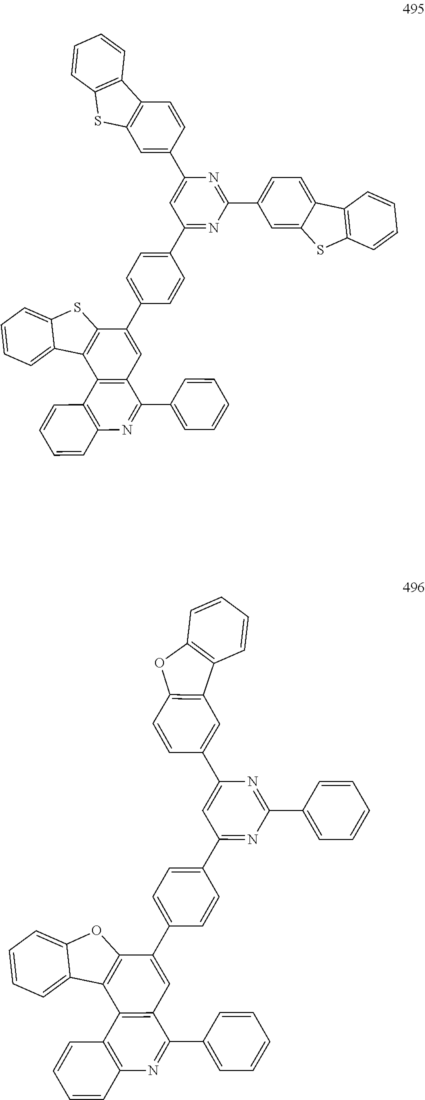 [SMILES: c1ccc(-c2nc3ccccc3c3c2cc(-c2ccc(-c4cc(-c5ccc6c(c5)sc5ccccc56)nc(-c5ccc6c(c5)sc5ccccc56)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3ccc4oc5ccccc5c4c3)n2)cc1]

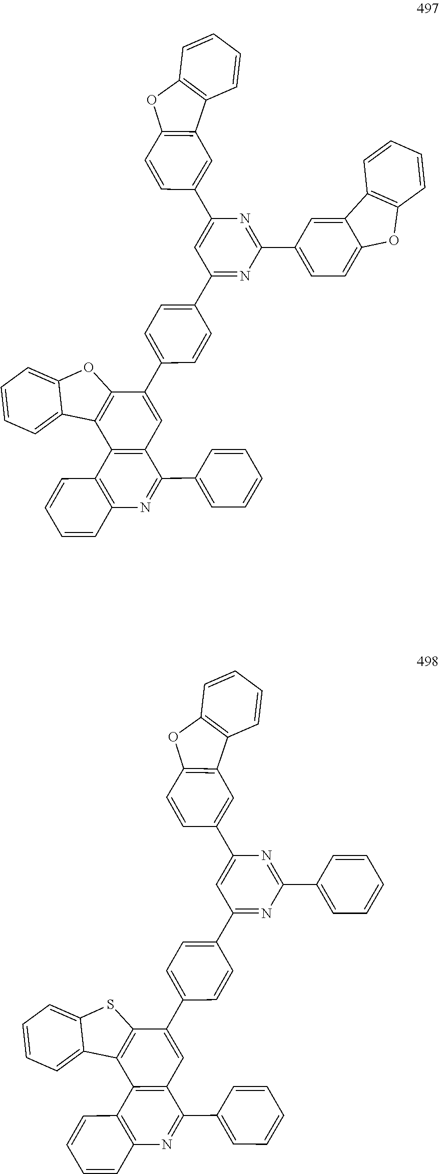 [SMILES: c1ccc(-c2nc3ccccc3c3c2cc(-c2ccc(-c4cc(-c5ccc6oc7ccccc7c6c5)nc(-c5ccc6oc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3ccc4oc5ccccc5c4c3)n2)cc1]

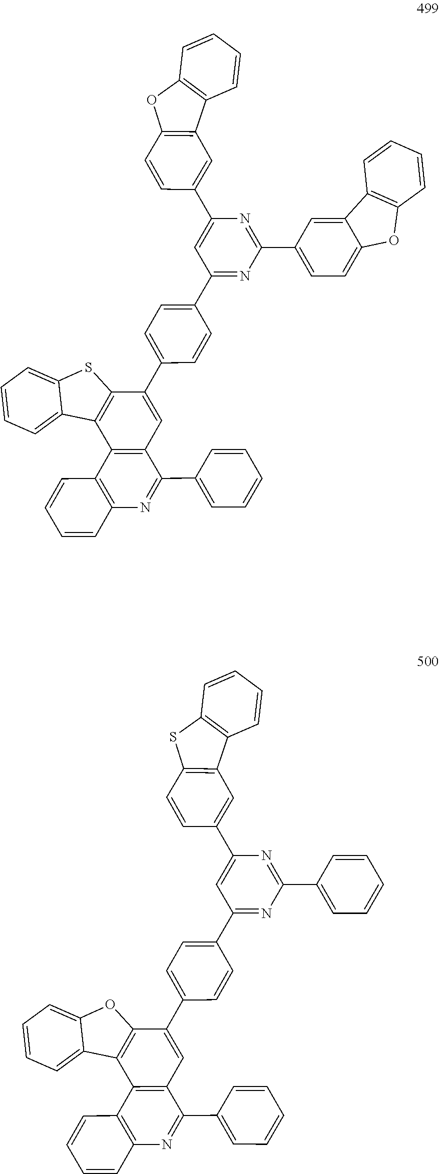 [SMILES: c1ccc(-c2nc3ccccc3c3c2cc(-c2ccc(-c4cc(-c5ccc6oc7ccccc7c6c5)nc(-c5ccc6oc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3ccc4sc5ccccc5c4c3)n2)cc1]

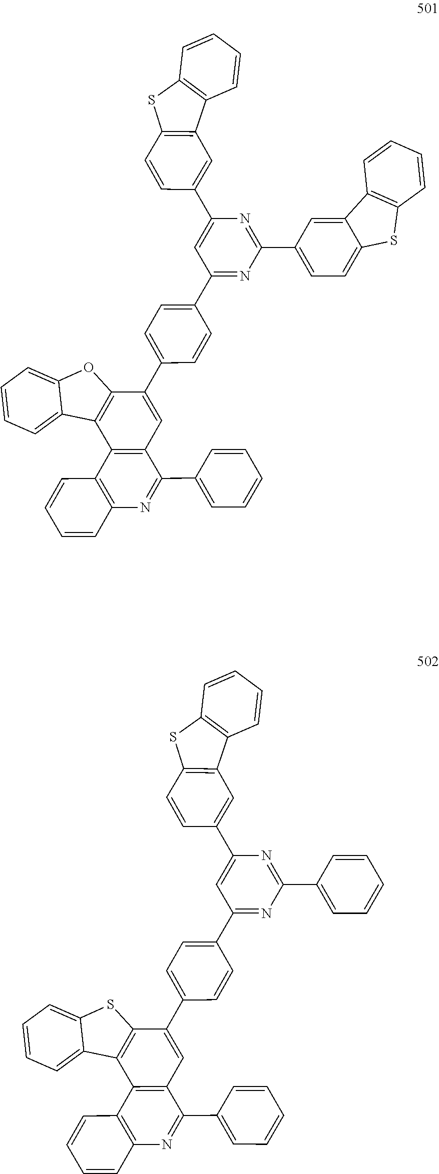 [SMILES: c1ccc(-c2nc3ccccc3c3c2cc(-c2ccc(-c4cc(-c5ccc6sc7ccccc7c6c5)nc(-c5ccc6sc7ccccc7c6c5)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3ccc4sc5ccccc5c4c3)n2)cc1]

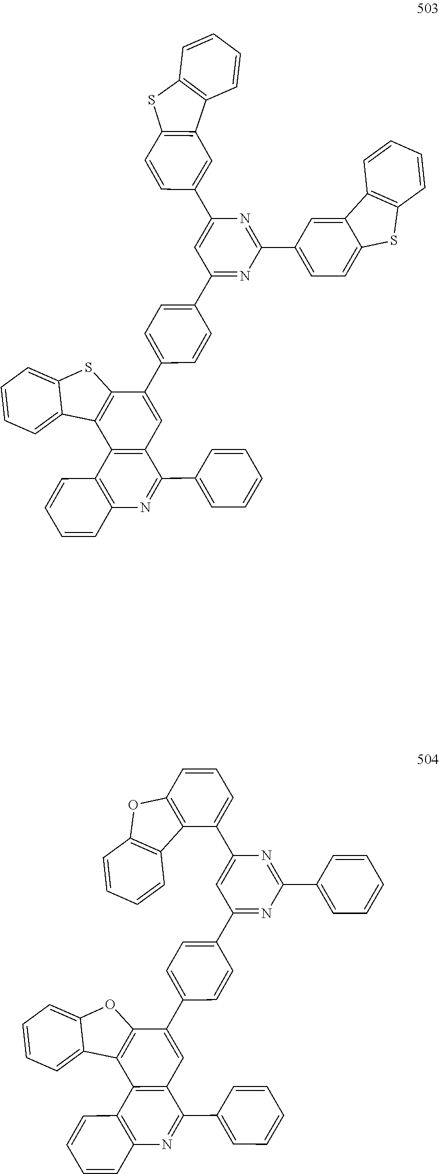 [SMILES: c1ccc(-c2nc3ccccc3c3c2cc(-c2ccc(-c4cc(-c5ccc6sc7ccccc7c6c5)nc(-c5ccc6sc7ccccc7c6c5)n4)cc2)c2sc4ccccc4c23)cc1 ; c1ccc(-c2nc(-c3ccc(-c4cc5c(-c6ccccc6)nc6ccccc6c5c5c4oc4ccccc45)cc3)cc(-c3cccc4oc5ccccc5c34)n2)cc1]

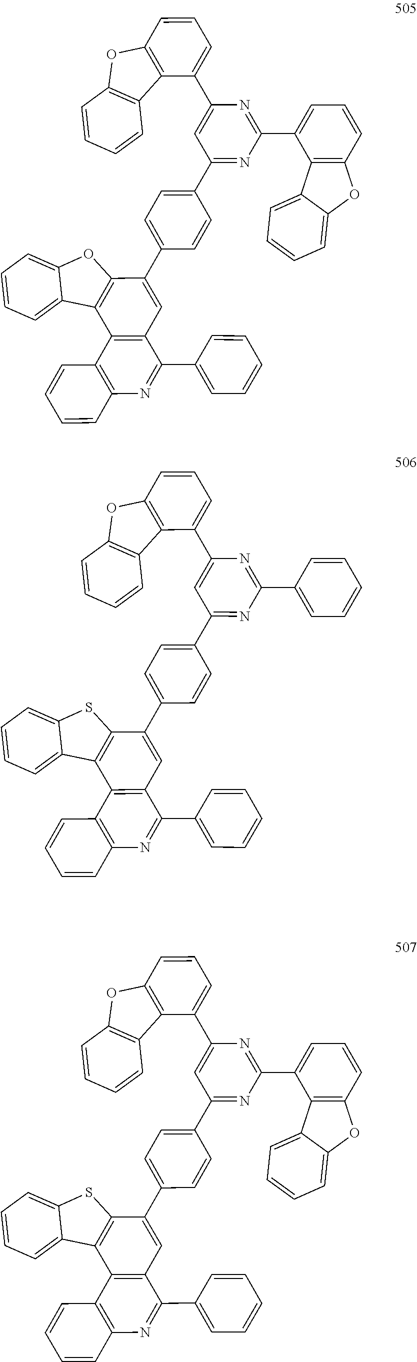 [SMILES: c1ccc(-c2nc3ccccc3c3c2cc(-c2ccc(-c4cc(-c5cccc6oc7ccccc7c56)nc(-c5cccc6oc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4oc5ccccc5c34)n2)cc1 ; c1ccc(-c2nc3ccccc3c3c2cc(-c2ccc(-c4cc(-c5cccc6oc7ccccc7c56)nc(-c5cccc6oc7ccccc7c56)n4)cc2)c2sc4ccccc4c23)cc1]

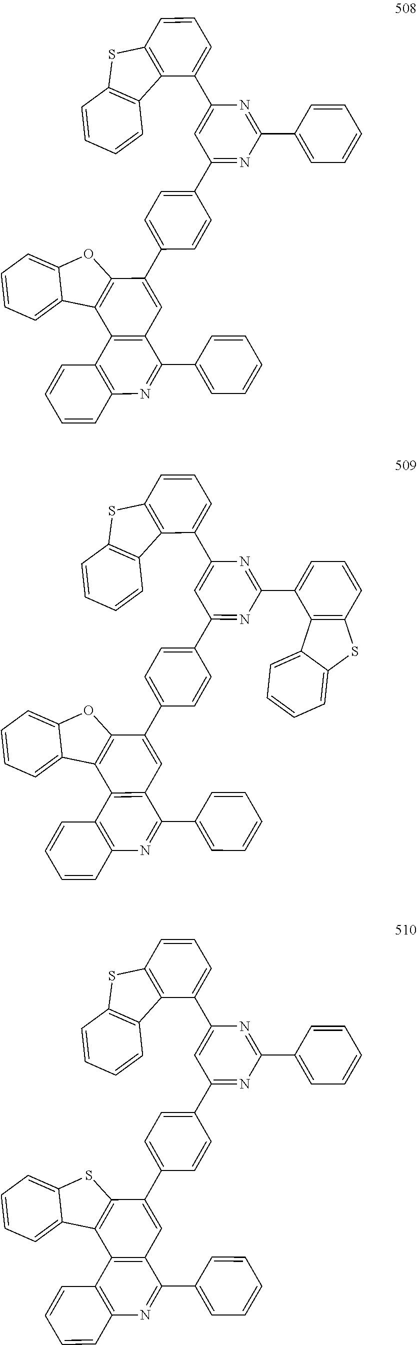 [SMILES: c1ccc(-c2nc(-c3ccc(-c4cc5c(-c6ccccc6)nc6ccccc6c5c5c4oc4ccccc45)cc3)cc(-c3cccc4sc5ccccc5c34)n2)cc1 ; c1ccc(-c2nc3ccccc3c3c2cc(-c2ccc(-c4cc(-c5cccc6sc7ccccc7c56)nc(-c5cccc6sc7ccccc7c56)n4)cc2)c2oc4ccccc4c23)cc1 ; c1ccc(-c2nc(-c3ccc(-c4cc5c(-c6ccccc6)nc6ccccc6c5c5c4sc4ccccc45)cc3)cc(-c3cccc4sc5ccccc5c34)n2)cc1]

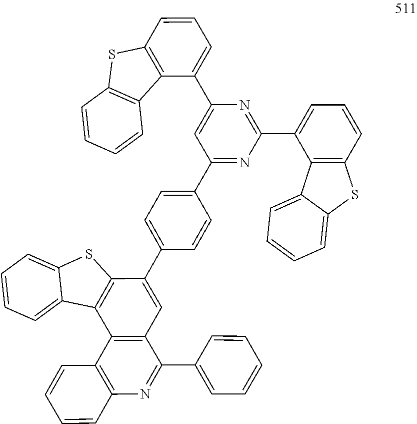 [SMILES: c1ccc(-c2nc3ccccc3c3c2cc(-c2ccc(-c4cc(-c5cccc6sc7ccccc7c56)nc(-c5cccc6sc7ccccc7c56)n4)cc2)c2sc4ccccc4c23)cc1]

7. An organic light emitting device comprising:
an anode, a cathode, and one or more layered organic material layers provided between the anode and the cathode,
wherein one or more layers of the organic material layers include the compound of claim 1.

8. The organic light emitting device of claim 7, wherein the organic material layer includes at least one layer of a hole blocking layer, an electron injecting layer, and an electron transport layer, and at least one layer of the hole blocking layer, the electron injecting layer, and the electron transport layer includes the compound.

9. The organic light emitting device of claim 7, wherein the organic material layer includes a light emitting layer, and the light emitting layer includes the compound.

10. The organic light emitting device of claim 9, wherein the compound is a light emitting host, and the light emitting layer further includes a light emitting dopant.

11. The organic light emitting device of claim 7, wherein the organic material layers include a charge generating layer, and the charge generating layer includes the compound.
